(19) United States
(12) Patent Application Publication
(10) Pub. No.: US 2025/0034150 A1
(43) Pub. Date: Jan. 30, 2025

(54) BTK INHIBITORS

(71) Applicant: BIOGEN MA INC., Cambridge, MA (US)

(72) Inventors: Brian T. Hopkins, Newton, MA (US); Bin Ma, Arlington, MA (US); Jürgen Schulz, Boon (DE); Marta Nevalainen, Hilliston, MA (US); TeYu Chen, Charlestown, MA (US); Robin Prince, Sharon, MA (US); Harold George Vandeveer, Stoneham, MA (US); Isaac Marx, Arlington, MA (US); Simone Sciabola, Wellesley, MA (US); Zain Yousaf, Quincy, MA (US)

(21) Appl. No.: 18/709,273

(22) PCT Filed: Nov. 10, 2022
(86) PCT No.: PCT/US2022/049618

§ 371 Date: May 10, 2024

Related U.S. Application Data
(60) Provisional application No. 63/277,879, filed on Nov. 10, 2021.

(51) Int. Cl. C07D 487/04; A61K 31/437; A61K 31/4985; A61K 31/519; A61K 31/5377; A61K 31/55; C07D 471/04; C07D 519/00

(52) U.S. Cl. CPC C07D 487/04; A61K 31/437; A61K 31/4985; A61K 31/519; A61K 31/5377; A61K 31/55; C07D 471/04; C07D 519/00

(57) ABSTRACT

Provided are compounds of Formula (I): or pharmaceutically acceptable salts thereof, wherein the variables in Formula (I) are as defined herein; and methods for their use and production.

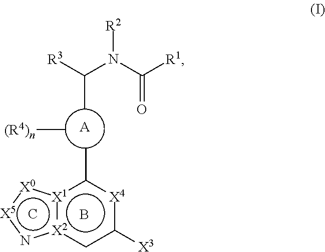

### RELATED APPLICATION

This application claims the benefit of the filing date, under 35 U.S.C. § 119(e), of U.S. Provisional Application No. 63/277,879, filed on Nov. 10, 2021, the entire contents of which are incorporated herein by reference.

### TECHNICAL FIELD

Provided are certain agents that inhibit Bruton's tyrosine kinase (Btk), and methods of making and using such agents.

### BACKGROUND

Protein kinases are a large multigene family consisting of more than 500 proteins which play a critical role in the development and treatment of a number of human diseases in oncology, neurology and immunology.

The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCy), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Given that Btk plays an important role in B-cell signaling, there is a great need in the art for effective inhibitors of Btk.

### SUMMARY

One aspect of the disclosure is a compound of Formula (I):

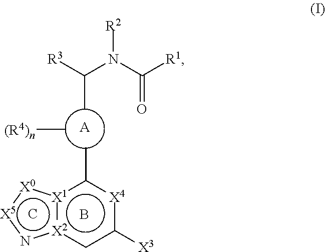

or a pharmaceutically acceptable salt thereof, wherein:
X⁰ is N, X¹ is C, X² is N, X⁴ is N and X⁵ is CH; X⁰ is CR⁰, X¹ is C, X² is N, X⁴ is N and X⁵ is CH; X⁰ is CR⁰, X¹ is N, X² is C, X⁴ is N and X⁵ is CH; X⁰ is CR⁰, X¹ is N, X² is C, X⁴ is CH and X⁵ is CH; X⁰ is CR⁰, X¹ is C, X² is N, X⁴ is CH and X⁵ is CH; or X⁰ is CH, X¹ is N, X² is C, X⁴ is CH and X⁵ is N;
X³ is H, —OR⁵, —N(R⁵)₂, 5- to 6-membered heteroaryl, or 4- to 7-membered monocyclic heterocyclyl, wherein the 5- to 6-membered heteroaryl and the 4- to 7-membered monocyclic heterocyclyl are optionally substituted with one or more (e.g., 1 to 6, 1 to 3, or 1, 2, 3, 4, 5 or 6) R⁵⁰;
R⁰ is H, halo, methyl, halomethyl, cyclopropyl or CN;
Ring A is phenyl, 5 or 6-membered heteroaryl or 5 to 10-membered monocyclic or bicyclic heterocyclyl;
R¹ is selected from —N(R^{1a})₂, —OR^{1a}, phenyl, 3- to 7-membered monocyclic carbocyclyl, 3- to 7-membered monocyclic heterocyclyl, 5- to 6-membered heteroaryl, 7- to 10-membered bicyclic carbocyclyl, 7- to 10-membered bicyclic heterocyclyl, and 8- to 10-membered bicyclic heteroaryl, wherein the phenyl, 3- to 7-membered monocyclic carbocyclyl, 3- to 7-membered monocyclic heterocyclyl, 5- to 6-membered heteroaryl, 7- to 10-membered bicyclic carbocyclyl, and 7- to 10-membered bicyclic heterocyclyl represented by R¹ are each optionally substituted with one or more (e.g., 1 to 6, 1 to 3, or 1, 2, 3, 4, 5 or 6) R¹⁰;
R^{1a}, for each occurrence, is independently selected from H, C₁₋₆ alkyl, C₂₋₆ alkenyl, C₂₋₆ alkynyl, phenyl, 3- to 7-membered carbocyclyl ring, 3- to 7-membered monocyclic heterocyclyl, and 5- to 6-membered heteroaryl, wherein the C₁₋₆ alkyl, C₂₋₆ alkenyl, C₂₋₆ alkynyl, phenyl, 3- to 7-membered carbocyclyl ring, 3- to 7-membered monocyclic heterocyclyl, and 5- to 6-membered heteroaryl represented by R^{1a} are each optionally substituted with one or more (e.g., 1 to 6, 1 to 3, or 1, 2, 3, 4, 5 or 6) R¹⁰;
R¹⁰, for each occurrence, is independently selected from halogen, —OR^{10a}, —S(O)₂R^{10a}, —CN, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R¹⁰ are each optionally substituted with one or more (e.g., 1 to 6, 1 to 3, or 1, 2, 3, 4, 5 or 6) R¹⁵;
R^{10a} is C₁₋₆ alkyl optionally substituted with one or more (e.g., 1 to 6, 1 to 3, or 1, 2, 3, 4, 5 or 6) halogen;
R¹⁵, for each occurrence, is independently selected from halogen, C₁₋₆ alkyl, C₁₋₆ haloalkyl, —CN, and —OR^{5a};
R^{15a} is C₁₋₆ alkyl;
R² is H, C₁₋₆ alkyl, C₂₋₆ alkenyl, and C₂₋₆ alkynyl;
or R¹ and R², together with their intervening atoms, form a Ring D selected from 3- to 7-membered monocyclic heterocyclyl, 7- to 10-membered bicyclic heterocyclyl, and 8- to 10-membered bicyclic heteroaryl, wherein Ring D is optionally substituted with one or more (e.g., 1 to 6, 1 to 3, or 1, 2, 3, 4, 5 or 6) R¹⁰⁰;
R¹⁰⁰, for each occurrence, is independently selected from C₁₋₆ alkyl, C₂₋₆ alkenyl, C₂₋₆ alkynyl, 3- to 7-membered monocyclic carbocyclyl, 4- to 6-membered monocyclic heterocyclyl and halogen; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R¹⁰⁰ are each optionally substituted with one or more (e.g., 1 to 6, 1 to 3, or 1, 2, 3, 4, 5 or 6) R¹⁵⁰;
R¹⁵⁰, for each occurrence, is independently selected from halogen, —OR^{150a}, 3- to 7-membered carbocyclyl ring and 4- to 7-membered monocyclic heterocyclyl;
R^{150a} is H or C₁₋₆ alkyl;
R³ is selected from H, halogen, —C(O)N(R^{3a})₂, —C(O)OR^{3a}, —C(O)R^{3a}, C₁₋₆ alkyl, C₂₋₆ alkenyl, and C₂₋₆ alkynyl, wherein the C₁₋₆ alkyl, C₂₋₆ alkenyl, and C₂₋₆ alkynyl represented by R³ are each optionally substituted with one or more (e.g., 1 to 6, 1 to 3, or 1, 2, 3, 4, 5 or 6) substituents selected from halogen and hydroxyl;
R^{3a}, for each occurrence, is independently selected from H, C₁₋₆ alkyl, C₂₋₆ alkenyl, C₂₋₆ alkynyl, phenyl, 3- to 7-membered carbocyclyl ring, 3- to 7-membered monocyclic heterocyclyl, or 5- to 6-membered heteroaryl, wherein C₁₋₆ alkyl, C₂₋₆ alkenyl, C₂₋₆ alkynyl, phenyl, 3- to 7-membered carbocyclyl ring, 3- to 7-membered monocyclic heterocyclyl, and 5- to 6-membered heteroaryl are optionally substituted with one or more (e.g., 1 to 6, 1 to 3, or 1, 2, 3, 4, 5 or 6) R³⁰;
or two R^{3a} groups on the same nitrogen are taken together with their intervening atoms to form a ring selected from 3- to 7-membered monocyclic heterocyclyl and 5- to 6-membered heteroaryl, wherein said ring is optionally substituted with one or more (e.g., 1 to 6, 1 to 3, or 1, 2, 3, 4, 5 or 6) R³⁰;
R³⁰, for each occurrence, is independently selected from halogen, —OR^{30a}, —N(R^{30a})₂, —C(O)N(R^{30a}), —C(O)₂R^{30a}, oxo, C₁₋₆ alkyl, C₂₋₆ alkenyl, C₂₋₆ alkynyl, 3- to 7-membered monocyclic carbocyclyl, and 4- to 6-membered monocyclic heterocyclyl;
R^{30a} is H or C₁₋₆ alkyl;
R⁴, for each occurrence, is independently selected from H, halogen, —NO₂, —CN, —OR^{4a}, —SR^{4a}, —N(R^{4a})₂, —C(O)R^{4a}, —C(O)OR^{4a}, —S(O)R^{4a}, —S(O)₂R^{4a}, —C(O)N(R^{4a})₂, —SO₂N(R^{4a})₂, —OC(O)R^{4a}, —N(R^{4a})C(O)R^{4a}, —N(R^{4a})C(O)OR^{4a}, —N(R^{4a})SO₂R^{4a}, —OC(O)N(R^{4a})₂, C₁₋₆ alkyl, C₂₋₆ alkenyl and C₂₋₆ alkynyl, wherein the C₁₋₆ alkyl, C₂₋₆ alkenyl and C₂₋₆ alkynyl are each optionally substituted with one or more (e.g., 1 to 6, 1 to 3, or 1, 2, 3, 4, 5 or 6) R⁴⁰;
R^{4a} is H, C₁₋₆ alkyl, C₂₋₆ alkenyl, C₂₋₆alkynyl, phenyl, 3- to 8-membered carbocyclyl ring, 3- to 7-membered monocyclic heterocyclyl, and 5- to 6-membered heteroaryl, wherein the C₁₋₆ alkyl, C₂₋₆ alkenyl, C₂₋₆ alkynyl, phenyl, 3- to 8-membered carbocyclyl ring, 3- to 7-membered monocyclic heterocyclyl, and 5- to 6-membered heteroaryl represented by R^{4a} are each optionally substituted with one or more (e.g., 1 to 6, 1 to 3, or 1, 2, 3, 4, 5 or 6) R⁴⁰; or two R^{4a} groups on the same nitrogen are taken together with their intervening atoms to form a ring selected from 3- to 7-membered monocyclic heterocyclyl and 5- to 6-membered heteroaryl, wherein said ring is optionally substituted with one or more (e.g., 1 to 6, 1 to 3, or 1, 2, 3, 4, 5 or 6) R⁴⁰;
R⁴⁰, for each occurrence, is independently selected from halogen, —OR^{40a}, —N(R^{40a})₂, —C(O)N(R^{40a})₂, —C(O)₂R^{40a}, oxo, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R⁴⁰ are each optionally substituted with one or more (e.g., 1 to 6, 1 to 3, or 1, 2, 3, 4, 5 or 6) R⁴⁵;
R^{40a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl are each optionally substituted with one or more (e.g., 1 to 6, 1 to 3, or 1, 2, 3, 4, 5 or 6) R⁴⁵;
R⁴⁵, for each occurrence, is independently selected from C₁₋₆ alkyl, halogen and —OR^{45a};
R^{45a} is H or C₁₋₆ alkyl;
or R³ and R⁴, together with their intervening atoms, form a Ring E, wherein Ring E is selected from 4- to 7-membered monocyclic carbocyclyl and 4- to 7-membered monocyclic heterocyclyl, wherein Ring E is optionally substituted with R³⁰⁰;
R³⁰⁰, for each occurrence, is independently selected from C₁₋₆ alkyl, C₂₋₆ alkenyl, C₂₋₆ alkynyl, 3- to 7-membered monocyclic carbocyclyl, 4- to 6-membered monocyclic heterocyclyl, halogen, —C(O)R^{300a}, —OR^{300a}, and —S(O)₂R^{300a}; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R³⁰⁰ are each optionally substituted with one or more (e.g., 1 to 6, 1 to 3, or 1, 2, 3, 4, 5 or 6) R³⁵⁰;
R^{300a} is selected from H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R^{300a} are each optionally substituted with one or more (e.g., 1 to 6, 1 to 3, or 1, 2, 3, 4, 5 or 6) R³⁵⁰;
R³⁵⁰, for each occurrence, is independently selected from C₁₋₆ alkyl, halogen, —CN, —C(O)R^{350a}, —C(O)N(R^{350a})₂, —N(R^{350a})₂, and —OR^{350a};
R^{350a}, for each occurrence, is independently H or C₁₋₆ alkyl optionally substituted with one to three halogen;
R⁵ is C₁₋₆ alkyl optionally substituted with one or more (e.g., 1 to 6, 1 to 3, or 1, 2, 3, 4, 5 or 6) substituents independently selected from halo, C₁₋₆ alkoxy, and C₁₋₆ haloalkoxy;
R⁵⁰, for each occurrence, is independently selected from halogen, —OR^{50a}, —N(R^{50a})₂, —C(O)N(R^{50a}), —C(O)₂R^{50a}, oxo, C₁₋₆ alkyl, 3- to 7-membered monocyclic carbocyclyl, and 4- to 6-membered monocyclic heterocyclyl, wherein the C₁₋₆alkyl, the 3- to 7-membered monocyclic carbocyclyl, and the 4- to 6-membered monocyclic heterocyclyl represented by R⁵⁰ are each optionally substituted with one or more substituents independently selected from C₁₋₆alkyl, CN, halo and C₁₋₆ alkoxy;
R^{50a} is H or C₁₋₆ alkyl;
n is 0, 1, 2, 3 or 4.

In a second aspect, the present disclosure provides a pharmaceutical composition comprising at least one compound described herein, or a pharmaceutically acceptable salt thereof, and a pharmaceutically acceptable excipient.

In a third aspect, the present disclosure provides methods of treating a disorder responsive to inhibition of Btk in a subject. The methods comprise administering to the subject an effective amount of at least one compound described herein, or a pharmaceutically acceptable salt thereof. The present disclosure also includes the use of at least one compound described herein, or a pharmaceutically acceptable salt thereof, for the manufacture of a medicament for the treatment of a disorder responsive to inhibition of Btk. Also provided is a compound described herein, or a pharmaceutically acceptable salt thereof for use in treating a disorder responsive to inhibition of Btk.

Other features or advantages will be apparent from the following detailed description of several embodiments, and also from the appended claims.

### DETAILED DESCRIPTION

The compounds or pharmaceutically acceptable salts thereof, as described herein, can have activity as Btk modulators. In particular, compounds or pharmaceutically acceptable salts thereof, as described herein, can be Btk inhibitors.

### I. Definitions

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The suffix “yl” added to the end of a chemical name indicates that the named moiety is bonded to the molecule at point. The suffix “ene” added to the end of a chemical name indicates that the named moiety is bonded to the molecule at two points. Examples include azetidinylene, pyrrolindinylene, piperidinylene, azapanylene or oxazapanylene, which indicates that an azetidine, pyrrolidine, piperidine, azapane or oxazapane is bonded to the remainder of the compound at two points.

As used herein, the term “alkyl” refers to a fully saturated branched or unbranched hydrocarbon moiety. In some embodiments, the alkyl comprises 1 to 20 carbon atoms, 1 to 10 carbon atoms, 1 to 8 carbon atoms, 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl.

“Alkenyl” refers to an unsaturated hydrocarbon group which may be linear or branched and has at least one carbon-carbon double bond. In some embodiments, alkenyl groups have 2 to 20 carbon atoms, 2 to 10 carbon atoms or 2-6 carbon atoms. The alkenyl group may contain 1, 2 or 3 carbon-carbon double bonds, or more. Examples of alkenyl groups include ethenyl, n-propenyl, iso-propenyl, n-but-2-enyl, n-hex-3-enyl and the like.

“Alkynyl” refers to an unsaturated hydrocarbon group which may be linear or branched and has at least one carbon-carbon triple bond. In some embodiments, alkynyl groups have 2 to 20 carbon atoms, 2 to 10 carbon atoms or 2-6 carbon atoms can be preferred. The alkynyl group may contain 1, 2 or 3 carbon-carbon triple bonds, or more. Examples of alkynyl groups include ethynyl, n-propynyl, n-but-2-ynyl, n-hex-3-ynyl and the like.

As used herein, the term “alkoxy” refers to a fully saturated branched or unbranched alkyl moiety attached through an oxygen bridge (i.e. a —O— C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. In some embodiments, alkoxy groups have about 1-4 carbons, more preferably about 1-2 carbons.

As used herein, the term “aryl” is defined to include all-carbon monocyclic or fused-ring polycyclic (i.e., rings which share adjacent pairs of carbon atoms) groups having a completely conjugated pi-electron system. An aryl group may have 6, 8, 9 or 10 carbon atoms in the ring(s). In some embodiments, an aryl group may have 6 or 10 carbon atoms in the ring(s). For example, as used herein, the term “(C₆-C₁₀)aryl” aromatic radicals containing from 6 to 10 carbon atoms such as phenyl, naphthyl, tetrahydronaphthyl, anthracenyl, indanyl and the like. An aryl group having 6 carbon atoms in the ring(s) may be optionally substituted by 1 to 5 suitable substituents.

In some embodiments, the number of carbon atoms in a group is specified herein by the prefix “Cₓ₋ₓₓ” or “Cₓ-Cₓₓ”, wherein x and xx are integers. For example, “C₁₋₄alkyl” is an alkyl group which has from 1 to 4 carbon atoms.

As used herein, the term “carbocyclyl”, “carbocycle” or “carbocyclic ring” refers to a saturated or partially unsaturated monocyclic or bicyclic (e.g., fused, bridged or spiro ring systems) ring system which has from 4- to 12-ring members, all of which are carbon. The term “carbocyclyl” encompasses cycloalkyl groups and cycloalkenyl groups.

In one embodiment, the carbocyclyl is a 3- to 7-membered monocyclic carbocyclyl.

Exemplary 3- to 7-membered monocyclic carbocyclyls include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl, phenyl and cycloheptatrienyl.

In one embodiment, the carbocyclyl is a 7- to 10-membered bicyclic carbocyclyl. Exemplary 7- to 10-membered bicyclic carbocyclyls include, but are not limited to, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, spiro[3.3]heptanyl, spiro[2.5]octanyl, spiro[2.2]pentanyl, spiro[3.3]heptanyl, bicyclo[3.3.0]octanyl, bicyclo[2.2.2]octanyl, bicyclo[3.3.1] nonanyl, bicyclo[3.3.2]decanyl, decalinyl, naphthyl and indanyl.

A fused bicyclic carbocyclyl has a 4 to 7 membered carbocyclyl fused to a 3 to 7 membered non-aromatic carbocyclyl. Examples include decahydronaphthalene, octahydro-1H-indene, octahydropentalene, decahydroazulene, decahydro-1H-annulene, bicycle[4.2.0]octane, bicycle[3.2.0]heptane and the like.

A bridged bicyclic carbocyclyl comprises a non-aromatic 5 to 7 membered carbocyclyl which shares three ring atoms with a 5 to 7 membered non-aromatic carbocyclyl.

Examples of bridged bicyclics carbocycles include bicyclo[2.2.1]hepantyl, bicyclo[3.2.1]octanyl, and bicyclo[3.3.1]nonanyl.

“Cycloalkyl” refers to completely saturated monocyclic hydrocarbon groups of 3-7 carbon atoms, including cyclopropyl, cyclobutyl, cyclopentyl, cyclohexyl and cyclopentyl; and “cycloalkyenyl” refers to unsaturated non-aromatic monocyclic hydrocarbon groups of 3-7 carbon atoms, including cyclopentenyl, cyclohexenyl and cyclopentenyl. The term “cycloalkyl” includes completely saturated monocyclic or bicyclic or spiro hydrocarbon groups of 3-7 carbon atoms, 3-6 carbon atoms, or 5-7 carbon atoms. In some embodiments, cycloalkyl is a 3- to 6-membered monocyclic cycloalkyl.

“Halogen” or “halo” may be fluoro, chloro, bromo or iodo.

The term “haloalkyl” or “halo-substituted alkyl” or refers to an alkyl group having at least one halogen substitution.

“Haloalkoxy” is a haloalkyl group which is attached to another moiety via an oxygen atom such as, e.g., but are not limited to —OCHCF₂ or —OCF₃.

“Heteroaryl” refers to an aromatic 5- to 6-membered monocyclic ring system, having 1 to 4 heteroatoms independently selected from O, N and S, and wherein N can be oxidized (e.g., N(O)) or quaternized, and S can be optionally oxidized to sulfoxide and sulfone.

Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithianyl, oxathianyl, triazinyl, tetrazinyl, and the like. In one embodiment, a heteroaryl is a 5-membered heteroaryl. Examples of a 5-membered heteroaryl include, but are not limited to, pyrazolyl, oxazolyl, isoxazolyl, 1,2,3-oxadiazolyl, 1,3,4-oxadiazolyl, 1,2,4-oxadiazolyl, 1,2,3-thiadiazolyl, 1,3,4-thiadiazolyl, 1,2,4-thiadiazolyl, 1,2,3-triazolyl, 1,2,4-triazolyl, and tetrazolyl.

“Heterocyclyl” refers to a saturated or partially unsaturated monocyclic or bicyclic (e.g., fused, bridged or spiro ring systems) ring system which has from 3- to 12-ring members, at least one of which is a heteroatom, and up to 4 (e.g., 1, 2, 3, or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. In some embodiments, the heterocyclyl is a 4- to 6-membered or 3- to 7-membered monocyclic heterocyclyl. In some embodiments, the heterocyclyl is a 7- to 10-membered bicyclic heterocyclyl, which can be fused, bridged or spiro bicyclic heterocyclyl. In some embodiments, the bicyclic heterocyclyl may include a non-aromatic heterocycle fused to a heteroaromatic ring.

Examples of monocyclic heterocyclyl include, but are not limited to, oxetanyl, thietanyl, azetidinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

A “fused ring system” has from 8 to 12 members (ring atoms) and two rings which share two adjacent ring atoms. A fused bicyclic heterocyclyl has a 4 to 7 membered heterocycyl fused to a 4 to 7 membered heterocycyl or a 3 to 7 membered carbocyclyl. A fused bicyclic heterocyclyl can also have a 4 to 7 membered heterocycyl fused to a 5 to 6 membered heteroaryl. Examples include cyclopentapyrrolidinyl, cyclopentapiperidinyl, cyclopentaazapanyl, cyclohexapyrrolidinyl, cyclohexapiperidinyl, cyclohexaazapanyl, cycloheptapyrrolidinyl, cycloheptapiperidinyl, cycheptaazapanyl, pyrrolopyrrolidinyl, pyrrolopiperidinyl, pyrroloazapanyl, furanopyrrolidinyl, furanopiperidinyl, furanoazapanyl, pyranopyrrolidinyl, pyranopiperidinyl, pyranoazapanyl, dihydropyrrolo[3,4-d]thiazoyl and the like.

A “bridged bicyclic ring system” (also referred to herein as a “bridged bicyclic” or “bridged ring system”) has 7 to 10 members (ring atoms) and two rings which share three adjacent ring atoms. A bridged bicyclic heterocyclyl comprises a 5 to 7 membered heterocycyl which shares three ring atoms with a 5 to 7 membered heterocycyl or a 5 to 7 membered carbocyclyl. Examples nitrogen containing bridged bicyclics include azabicyclo[2.2.1]hepantyl, azabicyclo[3.2.1]octanyl, azabicyclo[3.3.1]nonanyl, diazabicyclo[2.2.1]hepantyl, diazabicyclo[3.2.1]octanyl and diazabicyclo[3.3.1]nonanyl.

Examples of oxygen containing bridged bicyclics include oxobicyclo[2.2.1]hepantyl, oxobicyclo[3.2.1]octanyl, oxobicyclo[3.3.1]nonanyl, oxa-azabicyclo[2.2.1]hepantyl, oxa-azabicyclo[3.2.1]octanyl and oxa-azabicyclo[3.3.1]nonanyl.

A “spiro ring system” (also referred to herein as a “spirocycle”) has 8 to 12 members (ring atoms) and two rings which share one ring atom. A spirobicyclic heterocyclyl comprises a 4 to 7 membered heterocycyl which shares one atom with a 4 to 7 membered heterocycyl or a 4 to 7 membered non-aromatic carbocyclyl. Examples of 8 to 12 nitrogen containing spiro rings systems include 3,4-azabicyclooctanyl, 4,4-azabicyclononanyl, 3,5-azabicyclononanyl, 3,6-azabicyclodecanyl, 4,5-azabicyclodecanyl, 3,7-azabicycloundecanyl, 4,6-azabicycloundecanyl and 5,5-azabicycloundecanyl. Examples of 8-12 oxygen containing spiro ring systems include 3,4-oxobicyclooctanyl, 4,4-oxobicyclononanyl, 3,5-oxobicyclononanyl, 3,6-oxobicyclodecanyl, 4,5-oxobicyclodecanyl, 3,7-oxobicycloundecanyl, 4,6-oxobicycloundecanyl and 5,5-xobicycloundecanyl.

The term “oxo” refers to the diradical ═O.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers is at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. In addition, some compounds may exhibit polymorphism.

In one embodiment, the invention provides deuterated compounds disclosed herein, in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance. The compounds or pharmaceutically acceptable salts thereof as described herein, may exist in tautomeric forms and mixtures and separate individual tautomers are contemplated.

### II. Compounds of the Disclosure

In a first embodiment, the compound of the invention is represented by Formula (I) or a pharmaceutically acceptable salt thereof, wherein the variables are as described above.

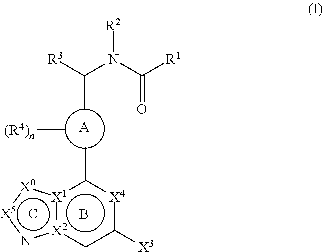

In a second embodiment of the disclosure, for compounds of formula (I), or a pharmaceutically acceptable salts thereof, X³ is —OR⁵, —N(R⁵)₂, 5- to 6-membered heteroaryl, or 4- to 7-membered monocyclic heterocyclyl, wherein the 5- to 6-membered heteroaryl and the 4- to 7-membered monocyclic heterocyclyl are optionally substituted with one or more (e.g., 1 to 6, 1 to 3, or 1, 2, 3, 4, 5 or 6) R⁵⁰, and the remaining variables are as described in the first embodiment.

In a third embodiment of the present disclosure, for compounds of formula (I), or a pharmaceutically acceptable salts thereof, R⁰ is H; and the remaining variables are as described in the first or second embodiment.

In a fourth embodiment of the present disclosure, for compounds of formula (I), or a pharmaceutically acceptable salt thereof, the compound is represented by the following formula:

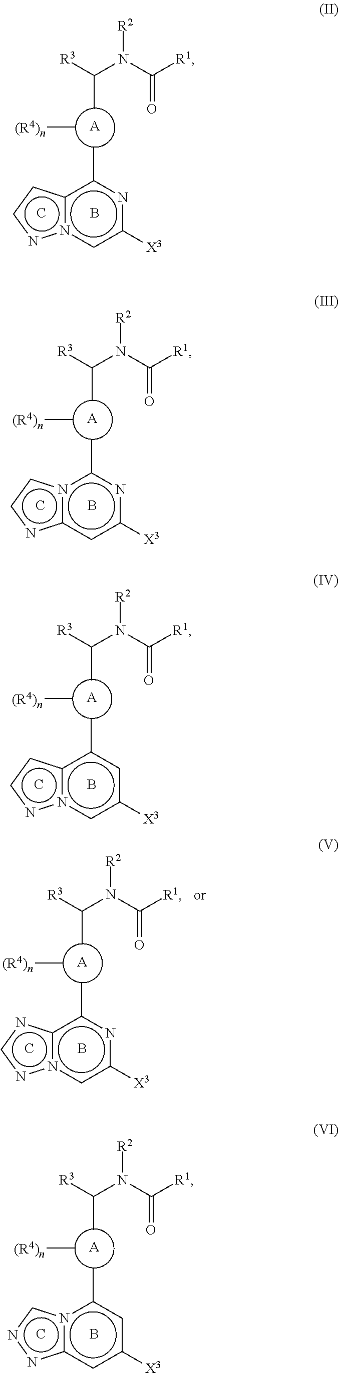

or pharmaceutically acceptable salts thereof; and the remaining variables are as described in the first or second embodiment.

In a fifth embodiment of the present disclosure, for compounds of formula (I), (II), (III), (IV), (V) or (VI), or a pharmaceutically acceptable salts thereof, X³ is —OR⁵, —N(R⁵)₂, 5-membered heteroaryl, or 4- to 6-membered monocyclic heterocyclyl, wherein the 5-membered heteroaryl and the 4- to 6-membered monocyclic heterocyclyl are optionally substituted with one to three R⁵⁰; and R⁵ is C₁₋₆ alkyl optionally substituted with C₁₋₆ alkoxy; and the remaining variables are as described in the first, second, third or fourth embodiment.

In a sixth embodiment of the present disclosure, for compounds of formula (I), (II), (III), (IV), (V) or (VI), or pharmaceutically acceptable salts thereof, X³ is selected from phenyl, azetidine, morpholine, oxadiazole, piperazine, pyrazole, tetrazole, each optionally substituted with one or two R⁵⁰; and the remaining variables are as described in the first, second, third, fourth or fifth embodiment.

In a seventh embodiment of the present disclosure, for compounds of formula (I), (II), (III), (IV), (V) or (VI), or pharmaceutically acceptable salts thereof, X³ is selected from:

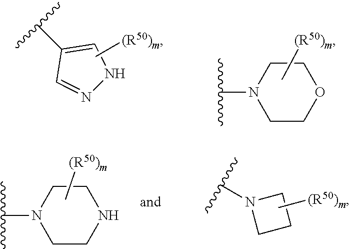

and m is 0, 1 or 2; and the remaining variables are as described in the first, second, third, fourth or fifth embodiment.

In an eighth embodiment of the present disclosure, for compounds of formula (I), (II), (III), (IV), (V) or (VI), or pharmaceutically acceptable salts thereof, X³ is selected from —O—CH₂—CH₂—OCH₃, —N(CH₃)₂,

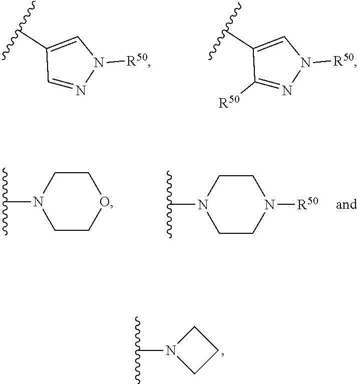

wherein [symbol] represents a bond to Ring B; and the remaining variables are as described in the [symbol] first, second, third, fourth or fifth embodiment.

In a ninth embodiment of the present disclosure, for compounds of formula (I), (II), (III), (IV), (V) or (VI), or pharmaceutically acceptable salts thereof, R⁵⁰ for each occurrence, is independently C₁₋₆ alkyl or a 4- to 6-membered monocyclic heterocyclyl, wherein the C₁₋₆alkyl represented by R⁵⁰ is optionally substituted with halo or CN; and the remaining variables are as described in the first, second, third, fourth, fifth, sixth, seventh or eighth embodiment.

In a tenth embodiment of the present disclosure, for compounds of formula (I), (II), (III), (IV), (V) or (VI), or pharmaceutically acceptable salts thereof, R⁵⁰, for each occurrence, is independently selected from —CH₃, —CH₂—CN and

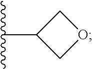 [SMILES: *C1COC1]

and the remaining variables are as described in the ninth embodiment.

In an eleventh embodiment of the present disclosure, for compounds of formula (I), (II), (III), (IV), (V) or (VI), or pharmaceutically acceptable salts thereof, R⁵⁰ is —CH₃; and the remaining variables are as described in the ninth embodiment.

In a twelfth embodiment of the present disclosure, for compounds of formula (I), (II), (III), (IV), (V) or (VI), or pharmaceutically acceptable salts thereof, Ring A is phenyl, 5 or 6-membered heteroaryl or 5 to 10-membered monocyclic or bicyclic heterocyclyl, each or which is optionally substituted with one to three R⁴; and the remaining variables are as described in the first, second, third, fourth, fifth, sixth, seventh, eighth, ninth, tenth or eleventh embodiment.

In a thirteenth embodiment of the present disclosure, for compounds of formula (I), (II), (III), (IV), (V) or (VI), or pharmaceutically acceptable salts thereof, Ring A is selected from 3-azabicyclo[3.2.1]octane, azepane, phenyl, piperidine, pyridine and pyrrolidine, each of which is optionally substituted with one to three R⁴; and the remaining variables are as described in the twelfth embodiment.

In a fourteenth embodiment of the present disclosure, for compounds of formula (I), (II), (III), (IV), (V) or (VI), or pharmaceutically acceptable salts thereof, A is selected from:

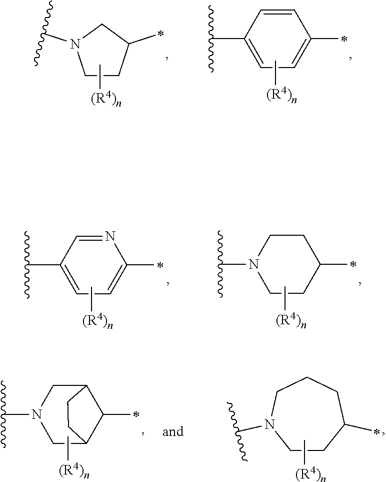

wherein [symbol] represents a bond to Ring B, and -* represents bond to

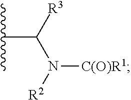

and the remaining variables are as described in the twelfth embodiment.

In a fifteenth embodiment of the present disclosure, for compounds of formula (I), (II), (III), (IV), (V) or (VI), or pharmaceutically acceptable salts thereof, n is 0, 1, 2; and the remaining variables are as described in the first, second, third, fourth, fifth, sixth, seventh, eighth, ninth, tenth, eleventh, twelfth, thirteenth or fourteenth embodiment.

In a sixteenth embodiment of the present disclosure, for compounds of formula (I), (II), (III), (IV), (V) or (VI), or pharmaceutically acceptable salts thereof, Ring A is selected from:

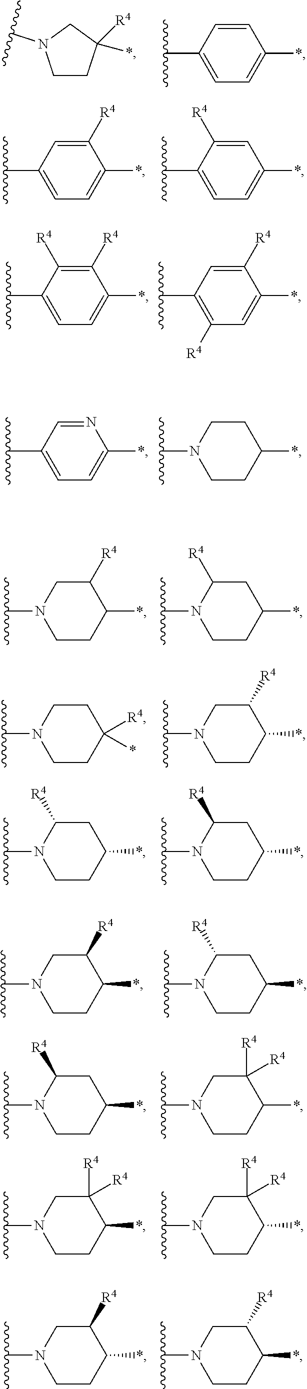

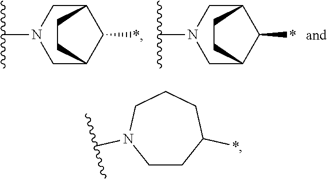

wherein [symbol] represents a bond to Ring B, and -* represents bond to

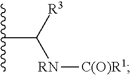

and the remaining variables are as described in the twelfth embodiment.

In a seventeenth embodiment of the present disclosure, for compounds of formula (I), (II), (III), (IV), (V) or (VI), or pharmaceutically acceptable salts thereof, R⁴ is selected from halogen, C₁₋₆ alkyl and C₁₋₆ haloalkyl; and the remaining variables are as described in the first, second, third, fourth, fifth, sixth, seventh, eighth, ninth, tenth, eleventh, twelfth, thirteenth, fourteenth, fifteenth or sixteenth embodiment.

In an eighteenth embodiment of the present disclosure, for compounds of formula (I), (II), (III), (IV), (V) or (VI), or pharmaceutically acceptable salts thereof, R⁴ is selected from Cl, F, —CH₃ and —CHF₂; and the remaining variables are as described in the seventeenth embodiment.

In eighteenth nineteenth embodiment of the present disclosure, for compounds of formula (I), (II), (III), (IV), (V) or (VI), or pharmaceutically acceptable salts thereof, R¹ is 5-membered heteroaryl optionally substituted with one or two R¹⁰; and the remaining variables are as described in the first, second, third, fourth, fifth, sixth, seventh, eighth, ninth, tenth, eleventh, twelfth, thirteenth, fourteenth, fifteenth, sixteenth, seventeenth or eighteenth embodiment.

In a twentieth embodiment of the present disclosure, for compounds of formula (I), (II), (III), (IV), (V) or (VI), or pharmaceutically acceptable salts thereof, R¹ is selected from oxazole, oxadiazole, pyrazole, tetrazole and triazole, each of which is optionally substituted with one or two R¹⁰; and the remaining variables are as described in the nineteenth embodiment.

In a twenty-first embodiment of the present disclosure, for compounds of formula (I), (II), (III), (IV), (V) or (VI), or pharmaceutically acceptable salts thereof, R¹ is selected from:

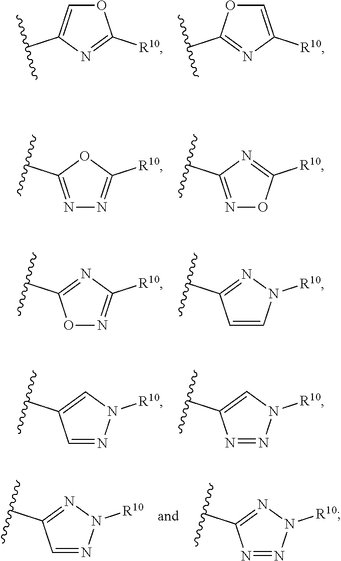

and the remaining variables are as described in the nineteenth embodiment.

In a twenty-second embodiment of the present disclosure, for compounds of formula (I), (II), (III), (IV), (V) or (VI), or pharmaceutically acceptable salts thereof, R¹⁰, for each occurrence, is independently selected from C₁₋₆ alkyl and C₃₋₆ cycloalkyl, each of which is optionally substituted with one to three R¹⁵; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-third embodiment of the present disclosure, for compounds of formula (I), (II), (III), (IV), (V) or (VI), or pharmaceutically acceptable salts thereof, R¹⁰, for each occurrence, is independently selected from C₁₋₄ alkyl and cyclopropyl, each of which is optionally substituted with one or three R¹⁵; and the remaining variables are as described in the twenty-second embodiment.

In a twenty-fourth embodiment of the present disclosure, for compounds of formula (I), (II), (III), (IV), (V) or (VI), or pharmaceutically acceptable salts thereof, R¹⁵, for each occurrence, is independently selected from halogen, C₁₋₄ alkyl and C₁₋₄ haloalkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second or twenty-third embodiment.

In a twenty-fifth embodiment of the present disclosure, for compounds of formula (I), (II), (III), (IV), (V) or (VI), or pharmaceutically acceptable salts thereof, R¹⁵, for each occurrence, is independently selected from F, —CH₃ and —CH₂F; and the remaining variables are as described in the twenty-fourth embodiment.

In a twenty-sixth embodiment of the present disclosure, for compounds of formula (I), (II), (III), (IV), (V) or (VI), or pharmaceutically acceptable salts thereof, R¹⁰, for each occurrence, is independently selected from —C(CH₃)₃, —C(CH₃)₂—CH₂F,

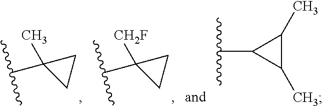

and CH₃; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-seventh embodiment of the present disclosure, for compounds of formula (I), (II), (III), (IV), (V) or (VI), or pharmaceutically acceptable salts thereof, R² is H; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth or twenty-sixth embodiment.

In a twenty-eighth embodiment of the present disclosure, for compounds of formula (I), (II), (III), (IV), (V) or (VI), or pharmaceutically acceptable salts thereof, R¹ and R², together with their intervening atoms, form a Ring D selected from 5- to 7-membered monocyclic heterocyclyl and 7- to 10-membered bicyclic heterocyclyl, wherein Ring D is optionally substituted with one or more (e.g., 1 to 6, 1 to 3, or 1, 2, 3, 4, 5 or 6) R¹⁰⁰; and the remaining variables are as described in the first, second, third, fourth, fifth, sixth, seventh, eighth, ninth, tenth, eleventh, twelfth, thirteenth, fourteenth, fifteenth, sixteenth, seventeenth or eighteenth embodiment.

In a twenty-ninth embodiment of the present disclosure, for compounds of formula (I), (II), (III), (IV), (V) or (VI), or pharmaceutically acceptable salts thereof, Ring D is selected from piperazinone and dihydropyrrolo[3,4-d]thiazolone, wherein Ring D is optionally substituted with one or two R¹⁰⁰; and the remaining variables are as described in the twenty-eighth embodiment.

In a thirtieth embodiment of the present disclosure, for compounds of formula (I), (II), (III), (IV), (V) or (VI), or pharmaceutically acceptable salts thereof, Ring D is selected from:

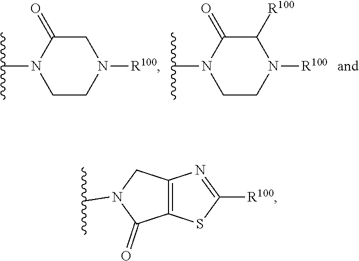

wherein [symbol] represents a bond to —C(R³)—Ring A; and the remaining variables are as described in the twenty-eighth embodiment.

In a thirty-first embodiment of the present disclosure, for compounds of formula (I), (II), (III), (IV), (V) or (VI), or pharmaceutically acceptable salts thereof, R¹⁰⁰, for each occurrence, is independently selected from C₁₋₆ alkyl and 4- to 6-membered monocyclic heterocyclyl, each of which is optionally substituted with one or two R¹⁵⁰; and R¹⁵⁰, for each occurrence, is independently selected from C₃₋₆ cycloalkyl and 4- to 6-membered monocyclic heterocyclyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth or thirtieth embodiment.

In a thirty-second embodiment of the present disclosure, for compounds of formula (I), (II), (III), (IV), (V) or (VI), or pharmaceutically acceptable salts thereof, R¹⁰⁰, for each occurrence, is independently selected from C₁₋₆ alkyl and oxetanyl, wherein the C₁₋₆ alkyl represented by R¹⁰⁰ is optionally substituted with R¹⁵⁰; and R¹⁵⁰, for each occurrence, is independently selected from cyclobutyl and oxetanyl; and the remaining variables are as described in the thirty-first embodiment.

In a thirty-third embodiment of the present disclosure, for compounds of formula (I), (II), (III), (IV), (V) or (VI), or pharmaceutically acceptable salts thereof, R¹⁰⁰, for each occurrence, is independently selected from —CH₃, —CH₂—CH₂—CH₃, —CH(CH₃)₂, —C(CH₃)₃, —CH₂—CH(CH₃)₂, —CH₂—C(CH₃)₃,

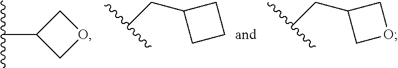

and the remaining variables are as described in the thirty-first embodiment.

In a thirty-fourth embodiment of the present disclosure, for compounds of formula (I), (II), (III), (IV), (V) or (VI), or pharmaceutically acceptable salts thereof, R³ is selected from H and C₁₋₆ alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second or thirty-third embodiment.

In a thirty-fifth embodiment of the present disclosure, for compounds of formula (I), (II), (III), (IV) or (V), or pharmaceutically acceptable salts thereof, R³ is H or —CH₃; and the remaining variables are as described in the thirty-fourth embodiment.

In a thirty-sixth embodiment of the present disclosure, for compounds of formula (I), (II), (III), (IV) or (V), or pharmaceutically acceptable salts thereof, R³ and R⁴, together with their intervening atoms, form a Ring E, wherein Ring E is selected from 4- to 7-membered monocyclic carbocycle and 4- to 7-membered monocyclic heterocycle, wherein Ring E is optionally substituted with R³⁰⁰; and the remaining variables are as described in the first, second, third, fourth, fifth, sixth, seventh, eighth, ninth, tenth, eleventh, twelfth, thirteenth, fourteenth, fifteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second or thirty-third embodiment.

In a thirty-seventh embodiment of the present disclosure, for compounds of formula (I), (II), (III), (IV) or (V), or pharmaceutically acceptable salts thereof, Ring E is 5- to 7-membered monocyclic heterocycle optionally substituted with R³⁰⁰; and the remaining variables are as described in the thirty-sixth embodiment.

In a thirty-eighth embodiment of the present disclosure, for compounds of formula (I), (II), (III), (IV) or (V), or pharmaceutically acceptable salts thereof, Ring E is represented by the following structural formula:

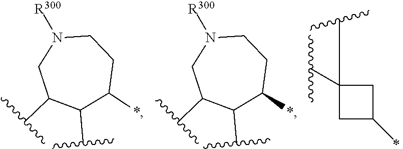

wherein [symbol] represents a point of fusion to Ring A and -* represents a bond to —N(R²)—C(O)—R¹; and the remaining variables are as described in the thirty-sixth embodiment.

In a thirty-ninth embodiment of the present disclosure, for compounds of formula (I), (II), (III), (IV) or (V), or pharmaceutically acceptable salts thereof, R³⁰⁰, for each occurrence, is independently selected from C₁₋₆ alkyl and 4- to 6-membered monocyclic heterocyclyl, wherein the C₁₋₆ alkyl and 4- to 6-membered monocyclic heterocyclyl represented by R³⁰⁰ are each optionally substituted with one to three R³⁵⁰; and R³⁵⁰, for each occurrence, is independently halogen; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh or thirty-eighth embodiment.

In a fortieth embodiment of the present disclosure, for compounds of formula (I), (II), (III), (IV) or (V), or pharmaceutically acceptable salts thereof, R³⁰⁰, for each occurrence, is independently selected from —CH₂—CF₃ and

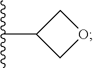 [SMILES: *C1COC1]

and the remaining variables are as described in the thirty-ninth embodiment.

In a forty-first embodiment of the present disclosure, the compound is represented by the following formula:

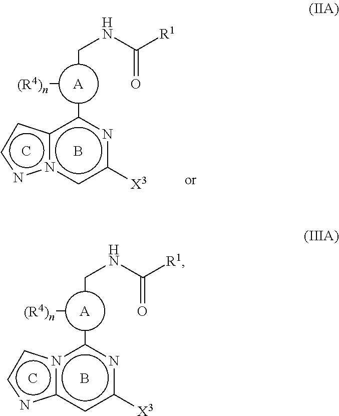

or a pharmaceutically acceptable salt thereof, wherein: R¹ is a 5-membered heteroaryl optionally substituted with R¹⁰; R¹⁰ is C₁₋₄ alkyl, C₁₋₄ haloalkyl or C₃₋₆ cycloalkyl optionally substituted with C₁₋₃ alkyl; X³ is a 5-membered heteroaryl or a 6-membered monocyclic saturated heterocyclyl, each of which is optionally substituted with R⁵⁰; R⁵⁰ is C₁₋₃ alkyl or C₁₋₃ haloalkyl; Ring A is phenyl, 6-membered heteroaryl or 6-membered monocyclic saturated heterocyclyl; R⁴, for each occurrence, is independently selected from halogen, C₁₋₃ alkyl and C₁₋₃ haloalkyl; and n is 0, 1 or 2.

In a forty-second embodiment of the present disclosure, for compounds of formula (IIA) or (IIIA), or pharmaceutically acceptable salts thereof, R¹ is selected from oxadiazole, triazole and tetrazole, each of which is optionally substituted with R¹⁰; X³ is pyrazole or piperazine, each of which is optionally substituted with R⁵⁰; and Ring A is phenyl, pyridine or piperidine; and the remaining variables are as described in the forty-first embodiment.

In a forty-third embodiment of the present disclosure, for compounds of formula (IIA) or (IIIA), or pharmaceutically acceptable salts thereof:
R¹ is selected from:

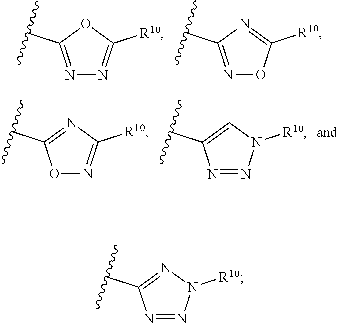

X³ is selected from:

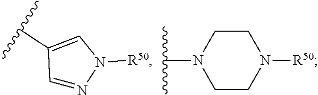

and
Ring A is selected from:

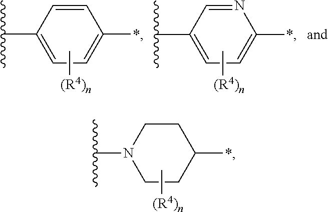

wherein [symbol] represents a bond to Ring B, and -* represents bond to

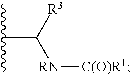

and the remaining variables are as described in the forty-first embodiment.

In a forty-fourth embodiment of the present disclosure, for compounds of formula (IIA) or (IIIA), or pharmaceutically acceptable salts thereof, ring A is selected from:

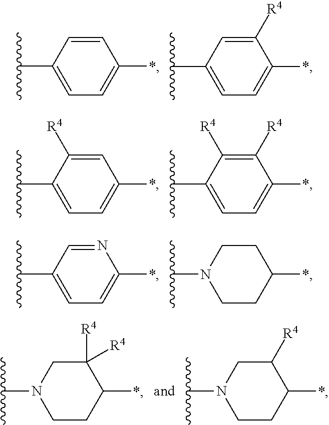

wherein [symbol] represents a bond to Ring B, and -* represents bond to

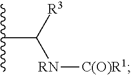

and the remaining variables are as described in the forty-third embodiment.

In a forty-fifth embodiment of the present disclosure, for compounds of formula (IIA) or (IIIA), or pharmaceutically acceptable salts thereof, R¹⁰ is

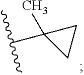 [SMILES: *C1(C)CC1]

R⁴, for each occurrence, is independently selected from F, —CH₃ and —CHF₂; and R⁵⁰ is —CH₃; and the remaining variables are as described in the forty-first, forty-second, forty-third or forty-fourth embodiment.

The invention also includes both the neutral form and pharmaceutically acceptable salts of the compounds disclosed in the exemplification (e.g., compounds of Examples 1-174).

### III. Pharmaceutical Composition and Methods of Uses

Another embodiment is a pharmaceutical composition comprising at least one compound described herein, or a pharmaceutically acceptable salt thereof, and at least one pharmaceutically acceptable carrier.

The compounds, or pharmaceutically acceptable salts thereof described herein may be used to decrease the activity of Btk, or to otherwise affect the properties and/or behavior of Btk, e.g., stability, phosphorylation, kinase activity, interactions with other proteins, etc.

In some embodiments, the present invention provides methods of decreasing Btk enzymatic activity. In some embodiments, such methods include contacting a Btk with an effective amount of a Btk inhibitor. Therefore, the present invention further provides methods of inhibiting Btk enzymatic activity by contacting a Btk with a Btk inhibitor of the present invention.

One embodiment of the invention includes a method of treating a disorder responsive to inhibition of Btk in a subject comprising administering to the subject an effective amount of at least one compound described herein, or a pharmaceutically acceptable salt thereof.

In one embodiment, the present invention provides methods of treating autoimmune disorders, inflammatory disorders, and cancers in a subject in need thereof comprising administering to the subject an effective amount of at least one compound described herein, or a pharmaceutically acceptable salt thereof.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis. The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present invention provides a method of treating rheumatoid arthritis or lupus. In some embodiments, the present invention provides a method of treating multiple sclerosis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating leukemia or lymphoma.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The a compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

The invention is illustrated by the following examples, which are not intended to be limiting.

### IV. Exemplifications

### A. Abbreviations and Acronyms

Abbreviations and acronyms used herein include the following:
ABPR: automated back pressure regulator;
Ac₂O: acetic anhydride;
ACN: acetonitrile;
Aq.: aqueous;
Ar: argon;
Bn: benzyl;
Boc: tert-butoxy carbonyl;
Boc₂O: di-tert-butyl decarbonate;
BPin: pinacolatoboron;
(BPin)₂ or B₂pin₂: bis(pinacolato)diboron, i.e., 4,4,4′,4′,5,5,5′,5′-Octamethyl-2,2′-bi-1,3,2-dioxaborolane;
br: broad;
t-BuOH: tert butanol;
n-BuLi: n-butyl lithium;
° C.: degrees Celsius;
CHCl₃: chloroform;
CDCl₃: deutero-chloroform;
CDI: 1,1′-Carbonyldiimiidazole
CO₂: carbon dioxide;
Cs₂CO₃: cesium carbonate;
CsF: cesium fluoride;
CuI: copper iodide;
δ: chemical shift;
d: doublet;
dd: double doublet;
ddd: double doublet of doublets;
DCM: dichloromethane;
DIEA or DIPEA: N-ethyldiisopropylamine or N,N-diisopropylethylamine;
DEA: diethylamine;
deg: degrees;
DIAD: diisopropyl azodicarboxylate;
DABCO: 1,4-Diazabicyclo[2.2.2]octane
DABAL-Me₃: adduct of trimethylaluminum and DABCO
DME: 1,2-dimethoxyethane;
DMF: N,N-dimethylformamide;
DMSO: dimethylsulfoxide;
DMSO-d₆: hexadeuterodimethyl sulfoxide;
DPPA: diphenylphosphoryl azide;
Et: ethyl;
Et₂O: ether;
EtOH: ethanol;
EtOAc: ethyl acetate;
Eq.: equivalent;
g: gram;
h: hour;
HATU: O-(7-azabenzotriazole-1-yl)-1,1,3,3-tetramethyluronium hexafluorophosphate;
HBr: hydrogen bromide;
HCl: hydrochloric acid;
HCO₂H: formic acid;
Hept: heptanes;
HFIP: hexafluoroisopropanol;
¹H NMR: proton nuclear magnetic resonance;
H₂O: water;
H₂SO₄: sulfuric acid;
HMPA: hexamethylphosphoramide;
HPLC: high pressure liquid chromatography;
Hz: Hertz;
IPA or iPrOH: iso-propanol;
J: coupling constant;
K₂CO₃: potassium carbonate;
kg: kilogram;
KHMDS: potassium hexamethyldisilazide;
KOAc: potassium acetate;
KOH: potassium hydroxide;
KOt-Bu: potassium tert-butoxide
K₃PO₄: potassium phosphate tribasic;
K₄Fe(CN)₆·3H₂O: potassium hexacyanoferrate (II) trihydrate;
L: liter;
LCMS: liquid chromatography mass spectrometry;
LG: leaving group;
m: multiplet;
M: molar;
MBPR: manual back pressure regulator;
Me: methyl;
MeB(OH)₂: methylboronic acid;
MeCN: acetonitrile;
MeOH: methanol;
MeOH-d₄: deutero-methanol;
mg: milligram;
MgSO₄: magnesium sulfate;
MHz: mega Hertz;
mins: minutes;
mL: milliliters;
mmol: millimole;
MMPNO: methylmorpholine N-oxide;
mol: mole;
MS m/z: mass spectrum peak;
N2: nitrogen;
NaOt-Bu: sodium tert-butoxide;
NaH: sodium hydride;
NaHCO₃: sodium bicarbonate;
NaHMDS: sodium hexamethyldisilylazide;
NaIO₄: sodium periodate;
NaOH: sodium hydroxide;
Na₂S₂O₃: sodium thiosulfate;
Na₂SO₄: sodium sulfate;
NEt₃: triethylamine;
NFSI: N-fluorobenzenesulfonimide;
NH₃: ammonia;
NH₄Cl: ammonium chloride;
NH₄OH: ammonium hydroxide;
NH₄OAc: ammonium acetate;
NIS: N-iodosuccinimide;
OsO₄: osmium tetroxide;
P(cy)₃: tricyclohexylphosphine;
Pd₂(dba)₃: tris(dibenzylideneacetone)dipalladium (0);
Pd(dppf)Cl₂: [1,1′-bis(diphenylphosphino)ferrocene]dichloropalladium(II);
Pd(dppf)Cl₂·DCM: [1,1′-Bis(diphenylphosphino)ferrocene]dichloropalladium(II),
complex with dichloromethane
Pd(dtbpf)Cl₂: [1,1′-bis(di-tert-butylphosphino)ferrocene]dichloropalladium(II);
Pd(t-Bu₃P)₂: Bis(tri-tert-butylphosphine)palladium(0);
PE: petroleum ether
PEPPSI-IPr or Pd-PEPPSI-IPr: [1,3-bis(2,6-diisopropylphenyl)imidazol-2-ylidene](3-chloropyridyl)palladium(II) dichloride
PG: protecting group;
Ph: phenyl;
POCl₃: phosphoryl chloride;
Pyr: pyridine;
q: quartet;
Rf: retardation factor;
rt: retention time;
RT: room temperature;
Rh(OAc)₂ dimer: Rhodium(II) acetate dimer;
RuPhos: 2-dicyclohexylphosphino-2′,6′-diisopropoxybiphenyl;
s: singlet;
sat.: saturated;
SCX: strong cation exchange;
SFC: supercritical fluid chromatography;
SiO₂: silicon dioxide;
Si-SPE: silica solid phase extraction;
SPE: solid phase extraction;
t: triplet;
td: triple doublet;
t-BuONa: sodium tert-butoxide;
TEA: triethylamine;
TFA: trifluoroacetic acid;
Tf₂O: Trifluoromethanesulfonic anhydride;
THF: tetrahydrofuran;
TLC: thin layer chromatography;
T₃P: propanephosphonic acid anhydride;
μL: microliters;
μmol: micromole;
ρW: microwave;
v/v: volume per volume;
Xphos: 2-dicyclohexylphosphino-2′,4′,6′-triisopropylbiphenyl;
Xphos G3: (2-dicyclohexylphosphino-2′,4′,6′-triisopropyl-1,1′-biphenyl)[2-(2′-amino-1,1′-biphenyl)]palladium(II) methanesulfonate;
tBuXPhos Pd G3: [(2-Di-tert-butylphosphino-2′,4′,6′-triisopropyl-1,1′-biphenyl)-2-(2′-amino-1,1′-biphenyl)] palladium(II) methanesulfonate

### B. General Synthetic Schemes

According to a first process, wherein the A ring is phenyl or 5 or 6-membered heteroaryl, compounds of Formula (I), may be prepared from compounds of Formulae (II′), (III′), (IV′) and (V′) as shown in Scheme 1.

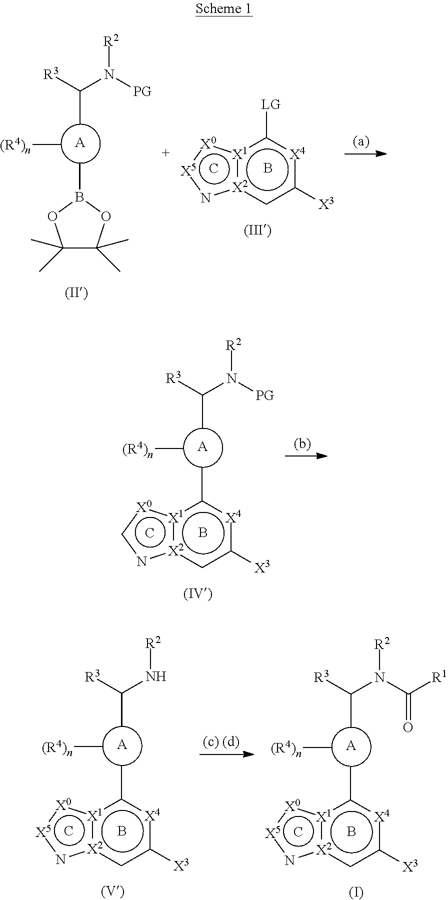

PG is a suitable N protecting group, typically a carbamate and preferable tert-butyl carbamate. LG is a leaving group, typically a halide, mesylate or triflate and preferably Br, Cl or triflate.

The compound of Formula (IV′) may be prepared from the compounds of Formulae (II′) and (III′) by process step a) a palladium catalysed, cross-coupling reaction, such as a Suzuki reaction. Typical cross-coupling reaction conditions comprise a palladium catalyst containing suitable phosphine ligands, in the presence of an inorganic base, in a suitable aqueous solvent at between RT and the reflux temperature of the reaction. Preferred conditions comprise, reaction of the compounds of Formulae (II′) and (III′), in the presence of tBuXPhos PdG3, Pd(t-Bu₃P)₂, Pd(dppf)Cl₂, or RuPhos in combination with Pd₂(dba)₃ and a suitable base such as Na₂CO₃, K₂CO₃, Cs₂CO₃, K₃PO₄ or KF in a suitable solvent such as aqueous dioxane at between 40° C. and 100° C.

The compound of Formula (V′) may be prepared from the compound of Formula (IV′) according to process step b) de-protection of a N protecting group, under acidic conditions. Preferred conditions comprise, reaction of the compound of Formula (IV′) with HCl or TFA in a suitable solvent such as MeOH, dioxane, EtOAc or DCM at between RT and 50° C.

The compound of Formula (I) may be prepared according to process step c) an amide bond formation, from the amine of Formula (V′) and R¹CO₂H, in the presence of a suitable coupling agent and organic base, in a suitable polar aprotic solvent. Preferred conditions comprise the reaction of the acid, R¹CO₂H with the amine of Formula (V′) in the presence of coupling agent, T3P®, HATU or CDI, in the presence of an organic base such as TEA or DIPEA, in a solvent, such as DMF, EtOAc, dioxane or MeCN at between RT and the reflux temperature of the reaction.

Alternatively, the compounds of Formula (I) may be prepared according to process step d), an amide bond formation, by reaction of the amine of Formula (V′) with R¹CO₂(C₁-C₄ alkyl) in the presence of DABAL-Me₃, according to the method described by Novak et al. (Tet. Lett. 2006, 47, 5767). Preferred conditions comprise reaction of the ester, R¹CO₂(C₁-C₄ alkyl) with the amine of Formula (V′) in the presence of DABAL-Me₃, in a suitable solvent such as THF or dioxane at between RT and 45° C.

According to a second process, wherein A ring is phenyl or 5 or 6-membered heteroaryl, compounds of Formula (IV′), may be prepared from compounds of Formulae (II′), (VI′) and (VII′) as shown in Scheme 2.

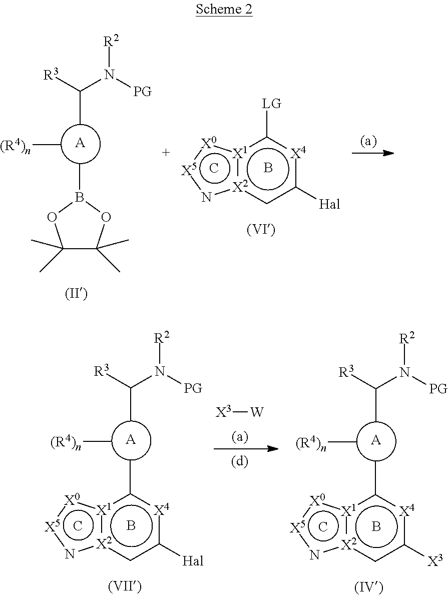

Hal is halogen, preferably Cl or Br, and W is a boronic acid or boronate ester.

The compound of Formula (VII′) may be obtained from the compounds of Formulae (II′) and (VI′) according to process step (a), a Suzuki reaction, as previously described in Scheme 1.

When X³ is 5- to 6-membered heteroaryl, the compound of Formula (IV) may be obtained from the compounds of Formula (VII′) and X³—W according to process step (a), a Suzuki reaction, as previously described in Scheme 1.

When X³ is —N(R⁵)₂, or an N-linked 4- to 7-membered monocyclic heterocyclyl, the compound of Formula (IV′) may be obtained by process step d) a Buchwald-Hartwig cross-coupling reaction. Typical conditions comprise, reaction of X³H with the compound of Formula (VII′) in the presence of a suitable inorganic base, a suitable palladium catalyst in the presence of suitable phosphine ligands, in a suitable solvent at elevated temperature. Preferred conditions comprise, reaction of the compounds of Formulae (VII′) and X³H in the presence of RuPhos in combination with Pd₂(dba)₃, in the presence of a suitable base such as K₂CO₃, K₃PO₄ or Cs₂CO₃ in a suitable solvent such as dioxane or toluene at between 70° C. and 110° C.

According to a third process, wherein the A ring is linked to the B ring through a N atom, the compound of Formula (IV′), may be prepared from compounds of Formulae (III′) and (VIII′) as shown in Scheme 3.

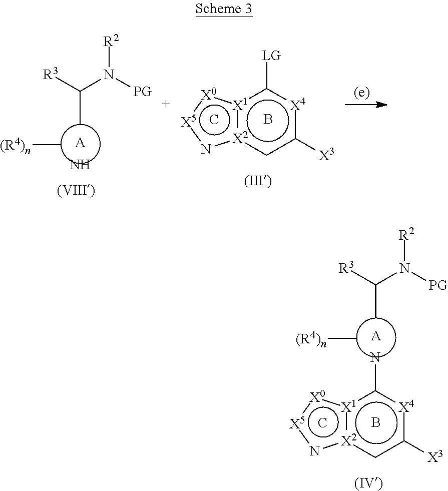

The compound of Formula (IV′) may be prepared according to process step e) an amination reaction. Typical conditions comprise reaction of the amine of Formula (VIII′) with the compound of Formula (III′), optionally in the presence of an organic or inorganic base, such as DIPEA or Cs₂CO₃ in IPA or DMF at RT.

According to a fourth process, the compound of Formula (I), may be prepared from the compounds of Formulae (II′), (VI′), (X′), (XI′) and (XII′) as shown in Scheme 4.

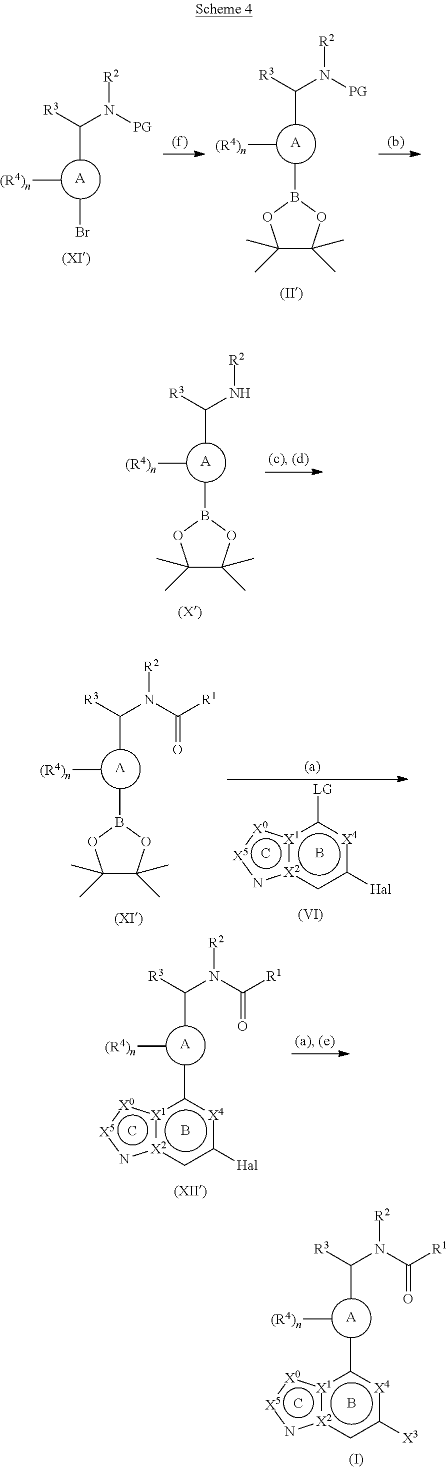

The compound of Formula (II′) may be prepared from the compound of Formula (XI′), according to process step (f), a boronate ester formation achieved by treatment with a suitable boronate such as (BPin)₂, in the presence of a suitable inorganic base, such as K₂CO₃ or KOAc and a suitable catalyst, such as, Pd(dppf)Cl₂ in a suitable non-polar solvent at between RT and elevated temperature. Preferred conditions comprise, treatment of the compound of Formula (II′) with (BPin)₂ in the presence of Pd(dppf)Cl₂ in the presence of KOAc in dioxane, at between RT and 90° C.

The compound of Formula (X′) may be prepared from the compound of Formula (II′) according to process step b) a deprotection reaction, as previously described in Scheme 1.

The compound of Formula (XI′) may be prepared from the compound of Formula (X′) and R¹CO₂H according to process step c) or R¹CO₂ (C₁-C₄)alkyl according to process step d) as previously described in Scheme 1.

The compound of Formula (XII′) may be prepared from the compounds of Formulae (XI′) and (VI′) according to process step a) as previously described in Scheme 1.

The compound of Formula (I) may be prepared from the compound of Formula (XII′) and X³H according to process steps a) or e) as previously described.

According to a fifth process, the compounds of Formula (XI′) may be prepared from the compound of Formula (XII′) as shown in Scheme 5.

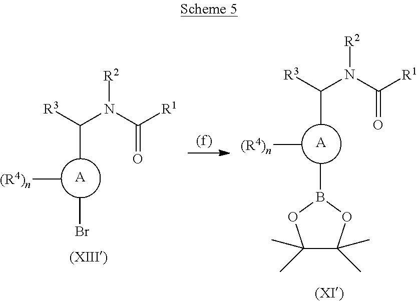

The compound of Formula (XI′) may be prepared from the compound of Formula (XII′) according to process step f) as previously described in Scheme 4.

According to a sixth process, wherein the A ring is linked to the B ring through a N atom, the compounds of Formula (XI′) may be prepared from the compound of Formula (XII′) as shown in Scheme 6.

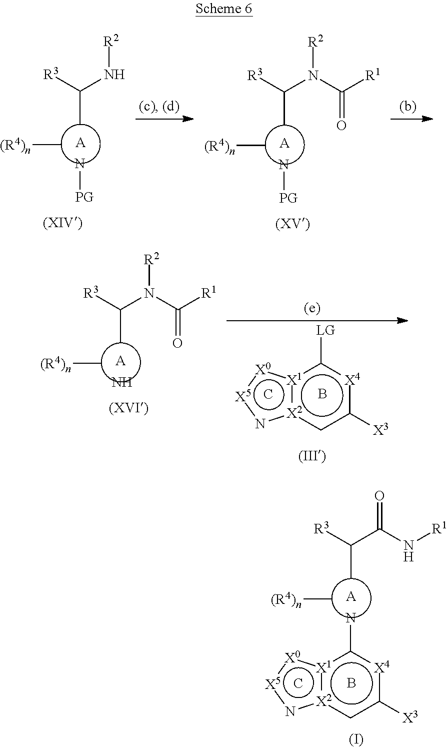

The compound of Formula (XV′) may be prepared from the compound of Formula (X′) and R¹CO₂H according to process step c) or R¹CO₂ (C₁-C₄)alkyl according to process step d) as previously described in Scheme 1.

The compound of Formula (XVI′) may be obtained from the compound of Formula (XVI′) according to process step b) as previously described in Scheme 1.

The compound of Formula (I) may be obtained from the compounds of Formulae (III′) and (XVI′) according to process step (e) as previously described in Scheme 3.

Compounds of Formulae (I) may be converted to alternative compounds of Formulae (I), by standard chemical transformations such as for example, alkylation of a heteroatom such as N, via reductive amination, or alkylation in the presence of an organic or inorganic base in a suitable solvent such as MeCN, using methods well known to those skilled in the art.

The compounds of Formulae (II′), (III′), (VI′), (VIII′), (XI′), (XIII′) and (XIV′) are commercially available, may be prepared by analogy to methods known in the literature, or the methods described in the Experimental section below.

It will be appreciated by those skilled in the art that it may be necessary to utilize a suitable protecting group strategy for the preparation of compounds of Formula (I). Typical protecting groups may comprise, carbamate and preferably Boc for the protection of amines.

It will be further appreciated that it may be necessary or desirable to carry out the transformations in a different order from that described in the schemes, or to modify one or more of the transformations, to provide the desired compound of the invention.

### C. Experimental

Preparative HPLC conditions

In the Example described below, the following preparative HPLC methods were used.

| Method | Conditions |
|---|---|
| A | Column: Waters XSelect CSH Prep C18 5 um OBD 19 × 100 mm, Mobile |
| | phase A: MeCN, Mobile phase B: H₂O, Modifier: 0.02% NH₄OH, |
| | Flow rate: 30 mL/min |
| A2 | Column: Waters XSelect CSH Prep C18 5 um OBD 50 × 100 mm, Mobile |
| | phase A: MeCN, Mobile phase B: H₂O, Modifier: 0.1% NH₄OH, |
| | Flow rate: 30 mL/min |
| B1 | Column: Waters XSelect CSH Prep C18 5 um OBD 19 × 100 mm, |
| | Mobile phase A: MeCN, Mobile phase B: H₂O, Modifier: 0.2% TFA |
| | Flow rate: 30 mL/min |
| B2 | Column: Waters XSelect CSH Prep C18 5 um OBD 19 × 100 mm, |
| | Mobile phase A: MeCN, Mobile phase B: H₂O, Modifier: 0.2% TFA |
| | Flow rate: 30 mL/min |
| C1 | Column: Waters Sunfire C18 5 um OBD 19 × 100 mm, |
| | Mobile phase A: MeCN, Mobile phase B: H₂O, Modifier: 0.1% TFA |
| | Flow rate: 30 mL/min |
| C2 | Column: Waters Sunfire C18 5 um OBD 19 × 100 mm, |
| | Mobile phase A: MeCN, Mobile phase B: H₂O, Modifier: 0.2% TFA |
| | Flow rate: 30 mL/min |
| C3 | Column: Waters Sunfire C18 5 um OBD 50 × 100 mm, |
| | Mobile phase A: MeCN, Mobile phase B: H₂O, Modifier: 0.1% TFA |
| D | Column: Phenomenex Synergi C18 150 × 30 mm × 4 um, |
| | Mobile Phase A: MeCN, Mobile phase B: H₂O (0.05% HCl) |
| | Flow Rate: 25 mL/min |
| E | Column: Welch Xtimate C18 150 × 30 mm x 5 um, |
| | Mobile phase A: MeCN, Mobile Phase B: H₂O (10 mM NH₄HCO₃) |
| | Flow Rate: 25 mL/min |
| F | Column: YMC-Actus Triart C18 100 × 30 mm x 5 um, |
| | Mobile phase A: MeCN, Mobile Phase B: H₂O (0.225% FA) |
| | Flow Rate: 25 mL/min |
| G | Column: Agela DuraShell C18 150 × 25 mm x 5 μm, |
| | Mobile Phase A: MeCN, Mobile phase B: 0.04% NH₃H₂O + 10 mM |
| | NH₄HCO₃ |
| | Flow Rate 25 mL/min |

C.1 Preparation of [1,5-a]pyrazines

### Example 1: 1-(tert-butyl)-N-(4-(6-(1-methyl-1H-pyrazol-4-yl)pyrazolo[1,5-a]pyrazin-4-yl)benzyl)-1H-pyrazole-3-carboxamide

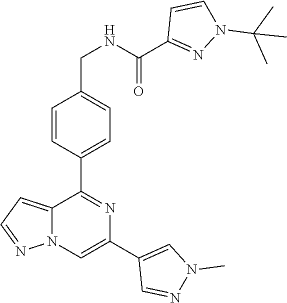 [SMILES: Cn1cc(-c2cn3nccc3c(-c3ccc(CNC(=O)c4ccn(C(C)(C)C)n4)cc3)n2)cn1]

### 1. Synthesis of diethyl 1-(2-(1-methyl-1H-pyrazol-4-yl)-2-oxoethyl)-1H-pyrazole-3,5-dicarboxylate

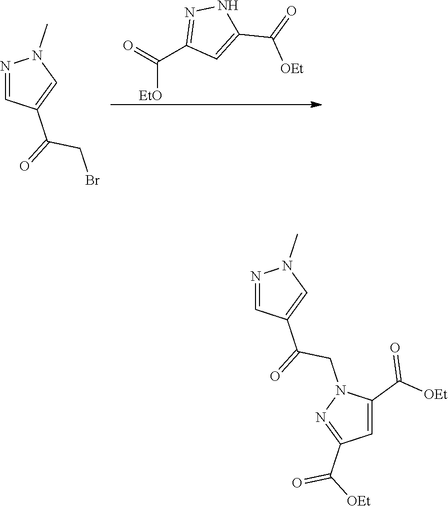

Diethyl 1H-pyrazole-3,5-dicarboxylate (69.6 g, 328 mmol) and Cs₂CO₃ (126 g, 388 mmol) were added to a solution of 2-bromo-1-(1-methyl-1H-pyrazol-4-yl)ethan-1-one (60.6 g, 298 mmol) in DMF (900 mL) and the reaction was stirred at RT overnight. The reaction mixture was diluted with water and extracted with DCM. The organic layer was dried (Na₂SO₄) and concentrated in vacuo. The crude product was suspended in heptanes: EtOAc 1:1 (50-100 mL) and filtered. The solid was washed with EtOAc and heptanes to give the product (68.5 g) as a white solid. The mother liquor was concentrated and purified by column chromatography (heptanes: EtOAc gradient 0 to 100%) to give additional product (9.7 g) to provide, 78.2 g (78% yield) of diethyl 1-(2-(1-methyl-1H-pyrazol-4-yl)-2-oxoethyl)-1H-pyrazole-3,5-dicarboxylate as a white solid. LCMS m/z=335.2 [M+H]⁺

### 2. Synthesis of ethyl 4-hydroxy-6-(1-methyl-1H-pyrazol-4-yl)pyrazolo[1,5-a]pyrazine-2-carboxylate

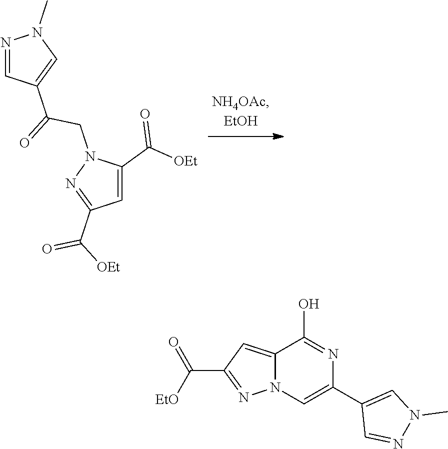 [SMILES: CCOC(=O)c1cc(C(=O)OCC)n(CC(=O)c2cnn(C)c2)n1 ; CCOC(=O)c1cc2c(O)nc(-c3cnn(C)c3)cn2n1]

A mixture of diethyl 1-(2-(1-methyl-1H-pyrazol-4-yl)-2-oxoethyl)-1H-pyrazole-3,5-dicarboxylate (15.0 g, 44.9 mmol) and NH₄OAc (10.4 g, 135 mmol) in EtOH (150 mL) was heated at 130° C. for 24 h in a Berghoff reactor. The resulting suspension was filtered, the solid washed with water, and dried to give ethyl 4-hydroxy-6-(1-methyl-1H-pyrazol-4-yl)pyrazolo[1,5-a]pyrazine-2-carboxylate (11.8 g, 92%) as a white solid. This reaction was performed batchwise on a total amount of 78.2 g of starting material, to provide a total of 66.6 g of product (92% yield). LCMS mz=288.3 [M+H]⁺

### 3. Synthesis of 4-hydroxy-6-(1-methyl-1H-pyrazol-4-yl)pyrazolo[1,5-a]pyrazine-2-carboxylic acid

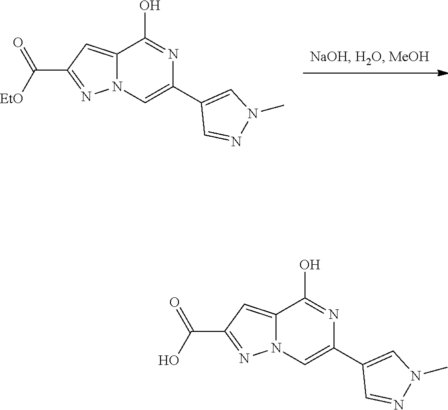 [SMILES: CCOC(=O)c1cc2c(O)nc(-c3cnn(C)c3)cn2n1 ; Cn1cc(-c2cn3nc(C(=O)O)cc3c(O)n2)cn1]

Ethyl 4-hydroxy-6-(1-methyl-1H-pyrazol-4-yl)pyrazolo[1,5-a]pyrazine-2-carboxylate (66.6 g, 232 mmol) was suspended in MeOH (1.2 L), 1M NaOH (696 mL, 696 mmol) was added and the reaction stirred at RT overnight. The mixture was acidified to pH 2 with conc. HCl and filtered. The solid was washed with MeOH, transferred to a round bottom flask and azeotroped with MeCN. The solid was split in two portions and the hydrolysis was repeated. Methyl 4-hydroxy-6-(1-methyl-1H-pyrazol-4-yl)pyrazolo[1,5-a]pyrazine-2-carboxylate (46.0 g, 116 mmol) was suspended in MeOH (1.2 L) and 1 M NaOH (348 mL, 348 mmol) and water (10 mL) were added and the reaction was stirred at RT overnight. The mixture was neutralized with conc. HCl, filtered and the solid was washed with MeCN, dioxane and dried under vacuum to give 4-hydroxy-6-(1-methyl-1H-pyrazol-4-yl)pyrazolo[1,5-a]pyrazine-2-carboxylic acid (71.0 g, crude) containing a lot of salts. The reaction was then repeated with the second portion to provide an additional 60 g of crude product. LCMS m/z=258.0 [M+H]⁺

### 4. Synthesis of 6-(1-methyl-1H-pyrazol-4-yl)pyrazolo[1,5-a]pyrazin-4-ol

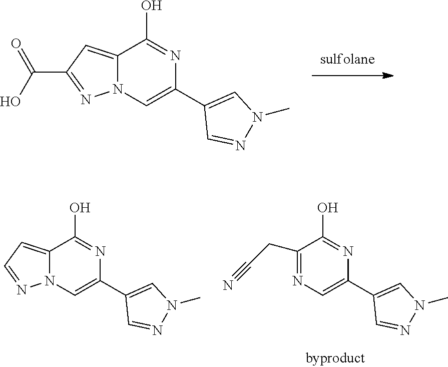

A three-necked flask was charged with pre-heated sulfolane (0.24 kg, 2.0 mol), which was heated to 50° C. 4-Hydroxy-6-(1-methyl-1H-pyrazol-4-yl)pyrazolo[1,5-a]pyrazine-2-carboxylic acid (41.8 g, 68 mmol) and a few drops of conc. sulfuric acid were added portion-wise and the reaction mixture was heated at 350° C. (external) for 4 h. The reaction was cooled to RT, diluted with DCM and purified by filtration through a short plug of silica eluting with 3 L of heptanes (fr1), 6 L heptanes: EtOAc 1:1 (fr2-3), 6 L EtOAc (fr4-5), 4 L DCM (fr6), 6 L DCM: MeOH 9:1 (fr7-8). The product (containing byproduct) (2.88 g, 20% yield) was isolated from fr7 as a brown solid. LCMS m/z=214.1 [M+H]⁺.

### 5. Synthesis of 4-chloro-6-(1-methyl-1H-pyrazol-4-yl)pyrazolo[1,5-a]pyrazine

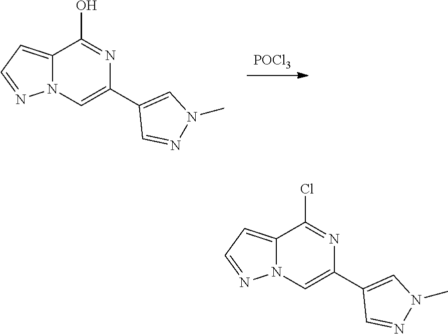 [SMILES: Cn1cc(-c2cn3nccc3c(O)n2)cn1 ; "POCl3" ; Cn1cc(-c2cn3nccc3c(Cl)n2)cn1]

6-(1-Methyl-1H-pyrazol-4-yl)pyrazolo[1,5-a]pyrazin-4-ol (2.88 g, 13.4 mmol) was suspended in POCl₃ (19.9 mL, 214 mmol) and the reaction was heated at 80° C. overnight. The mixture was diluted with MeCN and concentrated, the residue was suspended in DCM, and the mixture was washed with sat. NaHCO₃ and brine, dried over Na₂SO₄ and concentrated. The crude product was purified by column chromatography (DCM: EtOAc/TEA(5%); gradient 0 to 25%) to give 4-chloro-6-(1-methyl-1H-pyrazol-4-yl)pyrazolo[1,5-a]pyrazine (1.35 g, 43% yield) as a yellow solid. LCMS m/z=234.0 [M+H]⁺.

### 6. Synthesis of tert-butyl (4-(6-(1-methyl-1H-pyrazol-4-yl)pyrazolo[1,5-a]pyrazin-4-yl)benzyl)carbamate

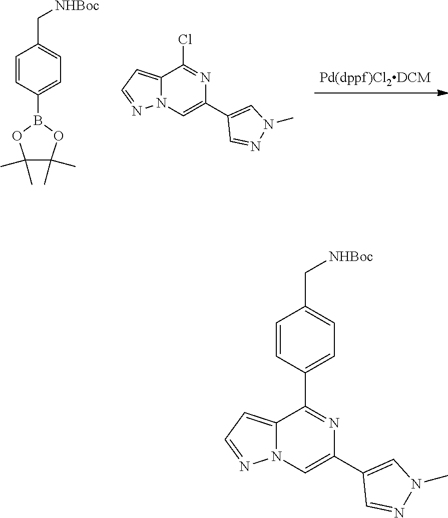

A mixture of tert-butyl (4-(4,4,5,5-tetramethyl-1,3,2-dioxaborolan-2-yl)benzyl)carbamate (300 mg, 900 μmol), 4-chloro-6-(1-methyl-1H-pyrazol-4-yl)pyrazolo[1,5-a]pyrazine (210 mg, 900 μmol), K₂CO₃ (373 mg, 2.70 mmol) and Pd(dppf)Cl₂•DCM (74 mg, 90 μmol) were dissolved in dioxane (7.2 mL) and water (1.8 mL) and N2 was bubbled through the mixture for 5 min. The reaction was heated to 100° C. under N2 and stirred at 100° C. for 3 h. The cooled reaction was concentrated and loaded directly onto a silica gel column. The crude material was purified via column chromatography (gradient: 0-75% [3:1 EtOAc/EtOH]: heptane) to afford tert-butyl (4-(6-(1-methyl-1H-pyrazol-4-yl)pyrazolo[1,5-a]pyrazin-4-yl)benzyl)carbamate as a white solid (264 mg, 73% yield). LCMS m/z=405.0 [M+H]⁺. 7. Synthesis of(4-(6-(1-methyl-1H-pyrazol-4-yl)pyrazolo[1,5-a]pyrazin-4-yl)phenyl)methanamine dihydrochloride

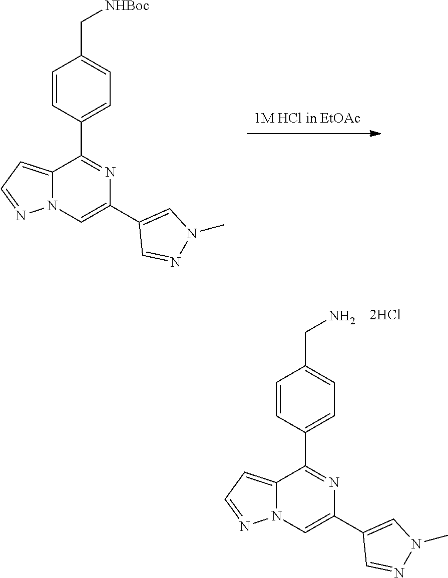 [SMILES: Cn1cc(-c2cn3nccc3c(-c3ccc(CNC(=O)OC(C)(C)C)cc3)n2)cn1 ; "1M HCl in EtOAc" ; Cn1cc(-c2cn3nccc3c(-c3ccc(CN)cc3)n2)cn1]

To a solution of tert-butyl (4-(6-(1-methyl-1H-pyrazol-4-yl)pyrazolo[1,5-a]pyrazin-4-yl)benzyl)carbamate (264 mg, 653 μmol) in EtOAc (1 mL) was added 1M HCl in EtOAc (6.53 mL, 6.53 mmol) and the reaction stirred for 3 days at ambient temperature. The reaction mixture was concentrated to dryness to give (4-(6-(1-methyl-1H-pyrazol-4-yl)pyrazolo[1,5-a]pyrazin-4-yl)phenyl)methanamine dihydrochloride, which was used without further purification (246 mg, crude). LCMS m/z=305.0 [M+H]⁺

### 8. Synthesis of 1-(tert-butyl)-N-(4-(6-(1-methyl-1H-pyrazol-4-yl)pyrazolo[1,5-a]pyrazin-4-yl)benzyl)-1H-pyrazole-3-carboxamide

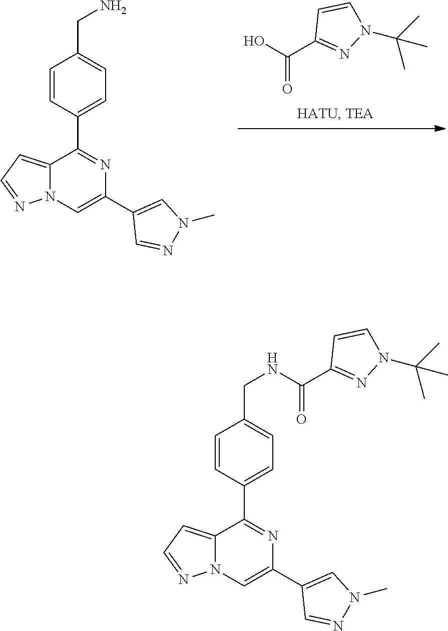 [SMILES: Cn1cc(-c2cn3nccc3c(-c3ccc(CN)cc3)n2)cn1 ; CC(C)(C)n1ccc(C(=O)O)n1 ; "HATU, TEA" ; Cn1cc(-c2cn3nccc3c(-c3ccc(CNC(=O)c4ccn(C(C)(C)C)n4)cc3)n2)cn1]

To a solution of 1-(tert-butyl)-1H-pyrazole-3-carboxylic acid (21 mg, 123 μmol) in THF (2 mL) in an ice water cooling bath was added TEA (33 mg, 329 μmol) and HATU (63 mg, 164 μmol). The mixture was stirred at 0° C. for 10 min, then (4-(6-(1-methyl-1H-pyrazol-4-yl)pyrazolo[1,5-a]pyrazin-4-yl)phenyl)methanamine dihydrochloride (25 mg, 82 μmol) was added. The reaction mixture was warmed to 23° C. and stirred for 24 h. The reaction was quenched with water (5 mL) then extracted with EtOAc (3×5 mL). The organic phases were combined, washed with brine, dried (Na₂SO₄), filtered and concentrated. The crude material was purified by reverse phase HPLC (Method A, 5-60% gradient) to give 1-(tert-butyl)-N-(4-(6-(1-methyl-1H-pyrazol-4-yl)pyrazolo[1,5-a]pyrazin-4-yl)benzyl)-1H-pyrazole-3-carboxamide as a beige solid (14.6 mg, 39% yield). LCMS m/z=455.1 [M+H]⁺. ¹H NMR (500 MHz, DMSO-d₆) δ: 9.12 (s, 1H), 8.66 (t, J=6.4 Hz, 1H), 8.33 (s, 1H), 8.18 (d, J=2.4 Hz, 1H), 8.15-8.08 (m, 3H), 7.92 (d, J=2.4 Hz, 1H), 7.54 (d, J=8.5 Hz, 2H), 7.22-7.13 (m, 1H), 6.66 (d, J=2.4 Hz, 1H), 4.55 (d, J=6.7 Hz, 2H), 3.95-3.83 (m, 3H), 1.57 (s, 9H).

### Example 2: Synthesis of 1-(tert-butyl)-N-(4-(6-(1-methyl-1H-pyrazol-4-yl)pyrazolo[1,5-a]pyrazin-4-yl)benzyl)-1H-pyrazole-4-carboxamide

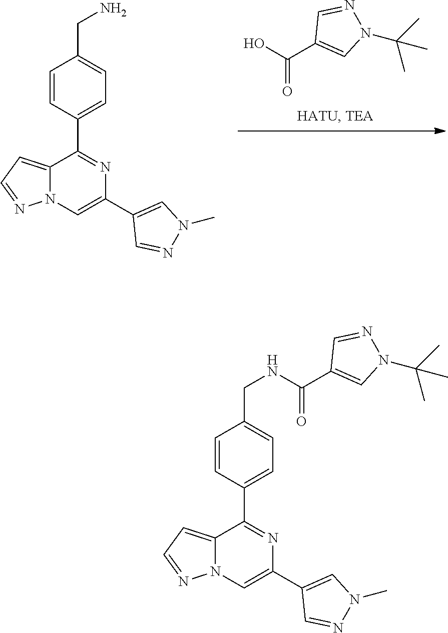 [SMILES: Cn1cc(-c2cn3nccc3c(-c3ccc(CN)cc3)n2)cn1 ; CC(C)(C)n1cc(C(=O)O)cn1 ; "HATU, TEA" ; Cn1cc(-c2cn3nccc3c(-c3ccc(CNC(=O)c4cnn(C(C)(C)C)c4)cc3)n2)cn1]

1-(Tert-butyl)-N-(4-(6-(1-methyl-1H-pyrazol-4-yl)pyrazolo[1,5-a]pyrazin-4-yl)benzyl)-1H-pyrazole-4-carboxamide was obtained as a beige solid (15.7 mg, 42% yield), from (4-(6-(1-methyl-1H-pyrazol-4-yl)pyrazolo[1,5-a]pyrazin-4-yl)phenyl)methanamine dihydrochloride (Example 1, step 7) and 1-(tert-butyl)-1H-pyrazole-4-carboxylic acid following a similar method to that described for Example 1, Step 8. The crude material was purified by reverse phase HPLC (Method A, 5-55% gradient). LCMS m/z=455.1 [M+H]⁺¹H NMR (500 MHz, DMSO-d₆) δ: 9.13 (s, 1H), 8.67 (t, J=5.8 Hz, 1H), 8.34-8.32 (m, 2H), 8.18 (d, J=2.4 Hz, 1H), 8.13-8.10 (m, 3H), 7.92 (s, 1H), 7.52 (d, J=8.5 Hz, 2H), 7.15 (d, J=1.8 Hz, 1H), 4.54 (d, J=6.1 Hz, 2H), 3.90 (s, 3H), 1.53 (s, 9H).

### Example 3: Synthesis of 2-(tert-butyl)-N-(4-(6-(1-methyl-1H-pyrazol-4-yl)pyrazolo[1,5-a]pyrazin-4-yl)benzyl)-2H-tetrazole-5-carboxamide

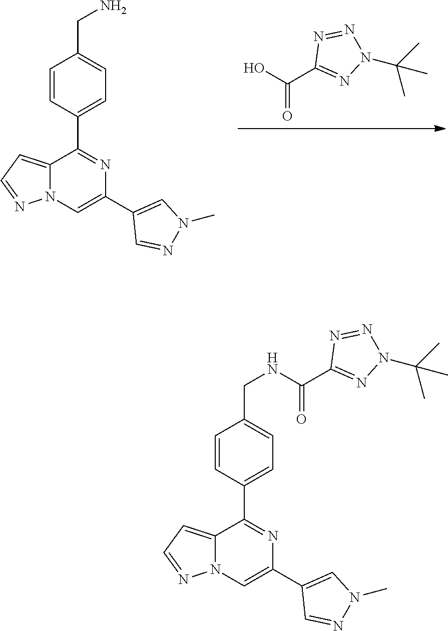 [SMILES: Cn1cc(-c2cn3nccc3c(-c3ccc(CN)cc3)n2)cn1 ; CC(C)(C)n1nnc(C(=O)O)n1 ; Cn1cc(-c2cn3nccc3c(-c3ccc(CNC(=O)c4nnn(C(C)(C)C)n4)cc3)n2)cn1]

2-(tert-Butyl)-N-(4-(6-(1-methyl-1H-pyrazol-4-yl)pyrazolo[1,5-a]pyrazin-4-yl)benzyl)-2H-tetrazole-5-carboxamide was obtained as a beige solid (17.7 mg, 47% yield) from (4-(6-(1-methyl-1H-pyrazol-4-yl)pyrazolo[1,5-a]pyrazin-4-yl)phenyl)methanamine dihydrochloride (Example 1, step 7) and 2-(tert-butyl)-2H-tetrazole-5-carboxylic acid following a similar method to that of Example 1, step 8. The crude material was purified by reverse phase HPLC (Method A, 5-55% gradient). LCMS m/z=457.1 [M+H]^{+ 1}H NMR (500 MHz, DMSO-d₆) δ: 9.67 (t, J=6.1 Hz, 1H), 9.13 (s, 1H), 8.33 (s, 1H), 8.18 (d, J=2.5 Hz, 1H), 8.14-8.10 (m, 3H), 7.56 (d, J=7.9 Hz, 2H), 7.16 (d, J=2.6 Hz, 1H), 4.60 (d, J=6.1 Hz, 2H), 3.90 (s, 3H), 1.74 (s, 9H).

### Example 4: Synthesis of 1-(tert-butyl)-N-(4-(6-(1-methyl-1H-pyrazol-4-yl)pyrazolo[1,5-a]pyrazin-4-yl)benzyl)-1H-1,2,3-triazole-4-carboxamide

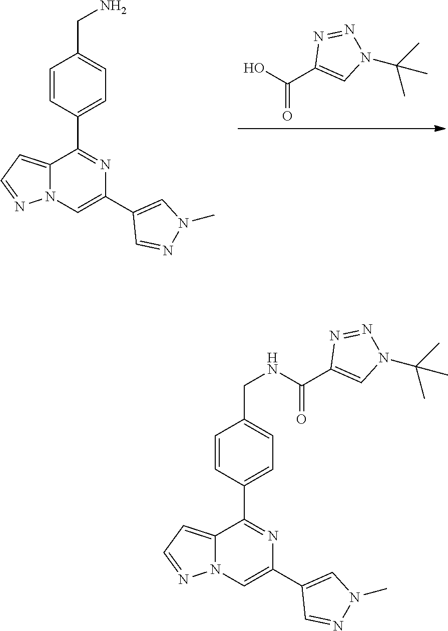 [SMILES: Cn1cc(-c2cn3nccc3c(-c3ccc(CN)cc3)n2)cn1 ; CC(C)(C)n1cc(C(=O)O)nn1 ; Cn1cc(-c2cn3nccc3c(-c3ccc(CNC(=O)c4cn(C(C)(C)C)nn4)cc3)n2)cn1]

1-(tert-Butyl)-N-(4-(6-(1-methyl-1H-pyrazol-4-yl)pyrazolo[1,5-a]pyrazin-4-yl)benzyl)-1H-1,2,3-triazole-4-carboxamide was obtained as a beige solid (18.2 mg, 49% yield), from (4-(6-(1-methyl-1H-pyrazol-4-yl)pyrazolo[1,5-a]pyrazin-4-yl)phenyl)methanamine dihydrochloride (Example 1, step 7) and 1-(tert-butyl)-1H-1,2,3-triazole-4-carboxylic acid following a similar procedure to that of Example 1, Step 8. The crude material was purified by reverse phase HPLC (Method A, 5-55% gradient). LCMS m/z=456.1 [M+H]⁺. ¹H NMR (500 MHz, DMSO-d₆) δ: 9.18 (t, J=6.0 Hz, 1H), 9.12 (s, 1H), 8.71 (s, 1H), 8.33 (s, 1H), 8.19-8.18 (m, 1H), 8.12-8.09 (m, 3H), 7.54 (d, J=8.5 Hz, 2H), 7.15 (d, J=2.7 Hz, 1H), 4.57 (d, J=6.1 Hz, 2H), 3.90 (s, 3H), 1.64 (s, 9H).

### Example 5: Synthesis of N-(4-(6-(1-methyl-1H-pyrazol-4-yl)pyrazolo[1,5-a]pyrazin-4-yl)benzyl)-5-(1-methylcyclopropyl)-1,2,4-oxadiazole-3-carboxamide

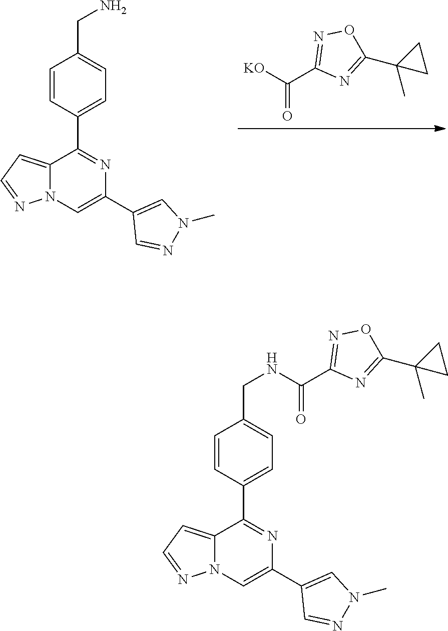

N-(4-(6-(1-methyl-1H-pyrazol-4-yl)pyrazolo[1,5-a]pyrazin-4-yl)benzyl)-5-(1-methylcyclopropyl)-1,2,4-oxadiazole-3-carboxamide was obtained as a yellow solid (13.2 mg, 35% yield) from (4-(6-(1-methyl-1H-pyrazol-4-yl)pyrazolo[1,5-a]pyrazin-4-yl)phenyl)methanamine dihydrochloride (Example 1, step 7) and potassium 5-(1-methylcyclopropyl)-1,2,4-oxadiazole-3-carboxylate following a similar procedure to that Example 1, step 8. The crude material was purified by reverse phase HPLC (Method A, 5-60% gradient). LCMS m/z=455.0 [M+H]⁺. ¹H NMR (500 MHz, DMSO-d₆) δ: 9.54 (t, J=6.1 Hz, 1H), 9.13 (s, 1H), 8.34 (s, 1H), 8.19 (d, J=2.7 Hz, 1H), 8.13-8.11 (m, 3H), 7.54 (d, J=8.5 Hz, 2H), 7.16 (d, J=1.8 Hz, 1H), 4.56 (d, J=6.1 Hz, 2H), 3.90 (s, 3H), 1.54 (s, 3H), 1.41-1.37 (m, 2H), 1.18-1.16 (m, 2H).

### Example 6. 5-(tert-butyl)-N-(2-fluoro-4-(6-(1-methyl-1H-pyrazol-4-yl)pyrazolo[1,5-a]pyrazin-4-yl)benzyl)-1,3,4-oxadiazole-2-carboxamide

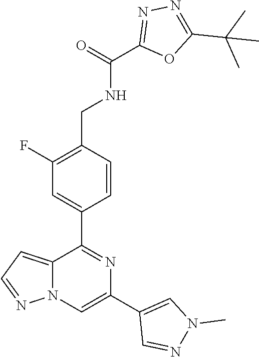 [SMILES: Cn1cc(-c2cn3nccc3c(-c3ccc(CNC(=O)c4nnc(C(C)(C)C)o4)c(F)c3)n2)cn1]

### 1. Preparation of tert-butyl (2-fluoro-4-(6-(1-methyl-1H-pyrazol-4-yl)pyrazolo[1,5-a]pyrazin-4-yl)benzyl)carbamate

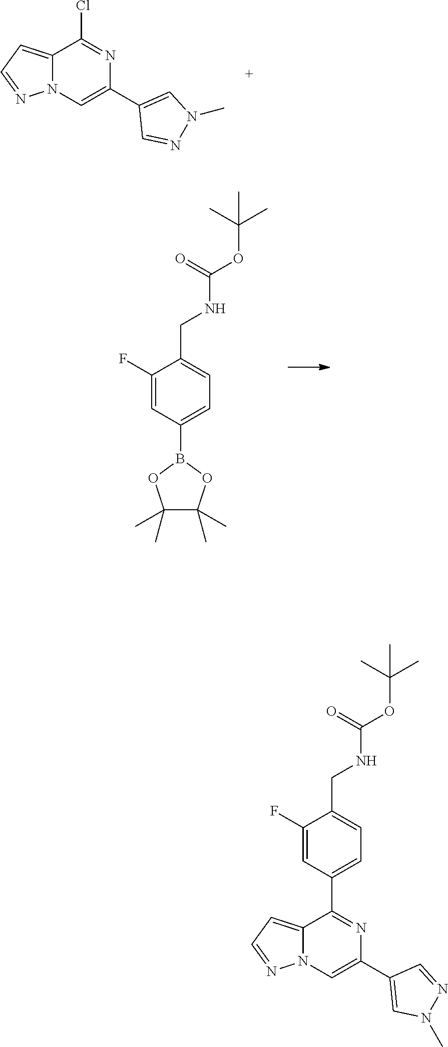 [SMILES: Cn1cc(-c2cn3nccc3c(Cl)n2)cn1 ; CC(C)(C)OC(=O)NCc1ccc(B2OC(C)(C)C(C)(C)O2)cc1F ; Cn1cc(-c2cn3nccc3c(-c3ccc(CNC(=O)OC(C)(C)C)c(F)c3)n2)cn1]

A mixture of tert-butyl (2-fluoro-4-(4,4,5,5-tetramethyl-1,3,2-dioxaborolan-2-yl)benzyl)carbamate (200 mg, 569.4 μmol), 4-chloro-6-(1-methyl-1H-pyrazol-4-yl)pyrazolo[1,5-a]pyrazine (Example 1, step 5, 133.06 mg, 569.4 μmol), K₂CO₃ (236.11 mg, 1.71 mmol), and Pd(dppf)Cl₂ (20.83 mg, 28.5 μmol) were dissolved in dioxane (4.56 mL) and water (1.14 mL) and N2 was bubbled through the mixture for 5 min. The reaction was heated to 100° C. under an atmosphere of N2 and stirred at 100° C. overnight. The cooled reaction was concentrated and purified by silica gel column chromatography (gradient elution 0-100% EtOAc:Heptane) to afford tert-butyl (2-fluoro-4-(6-(1-methyl-1H-pyrazol-4-yl)pyrazolo[1,5-a]pyrazin-4-yl)benzyl)carbamate (218 mg, 90.6% yield) as a light yellow solid. LCMS m/z=432.2 [M+H]⁺.

### 2. Preparation of(2-fluoro-4-(6-(1-methyl-1H-pyrazol-4-yl)pyrazolo[1,5-a]pyrazin-4-yl)phenyl)methanamine dihydrochloride

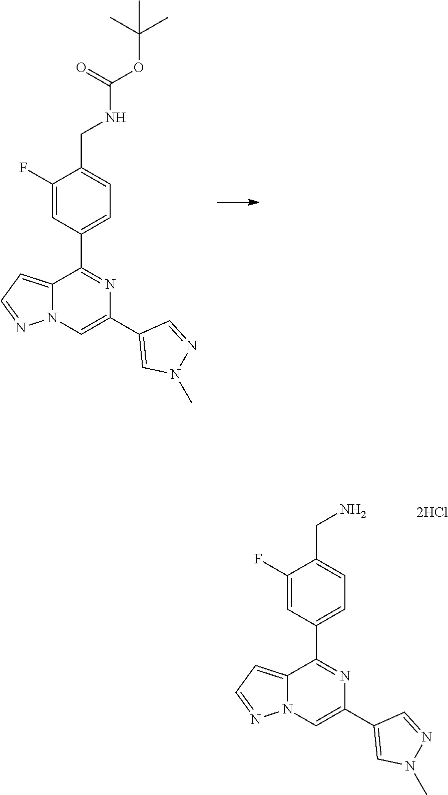 [SMILES: Cn1cc(-c2cn3nccc3c(-c3ccc(CNC(=O)OC(C)(C)C)c(F)c3)n2)cn1 ; Cn1cc(-c2cn3nccc3c(-c3ccc(CN)c(F)c3)n2)cn1]

tert-Butyl (2-fluoro-4-(6-(1-methyl-1H-pyrazol-4-yl)pyrazolo[1,5-a]pyrazin-4-yl)benzyl)carbamate (218 mg, 516.0 μmol) was dissolved in MeOH (5.16 mL) and HCl solution (1.25M in MeOH, 4.13 mL, 5.17 mmol) added and the reaction was stirred overnight at 50° C. The reaction was concentrated under vacuum and dried to afford (2-fluoro-4-(6-(1-methyl-1H-pyrazol-4-yl)pyrazolo[1,5-a]pyrazin-4-yl)phenyl)methanamine dihydrochloride (208 mg, crude) as a yellow solid. LCMS m/z=323.1 [M+H]⁺.

### 3. Preparation of 5-(tert-butyl)-N-(2-fluoro-4-(6-(1-methyl-1H-pyrazol-4-yl)pyrazolo[1,5-a]pyrazin-4-yl)benzyl)-1,3,4-oxadiazole-2-carboxamide

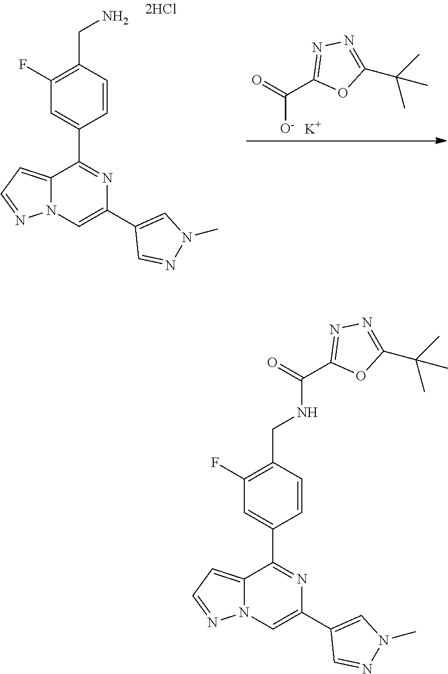 [SMILES: Cn1cc(-c2cn3nccc3c(-c3ccc(CN)c(F)c3)n2)cn1 ; CC(C)(C)c1nnc(C(=O)[O-])o1 ; [K+] ; Cn1cc(-c2cn3nccc3c(-c3ccc(CNC(=O)c4nnc(C(C)(C)C)o4)c(F)c3)n2)cn1]

A stock solution of (2-fluoro-4-(6-(1-methyl-1H-pyrazol-4-yl)pyrazolo[1,5-a]pyrazin-4-yl)phenyl)methanamine dihydrochloride (320 mg), DCM (8 mL), and DIPEA (1.5 mL) was prepared. The stock solution (1.2 mL) was added to a vial containing potassium 5-tert-butyl-1,3,4-oxadiazole-2-carboxylate (25 mg, 121.4 μmol) which was then cooled to 0° C. HATU (52 mg) was added, and the reaction stirred overnight at RT. The reaction mixture was diluted with DCM (2 mL) and water (2 mL), the vials shaken, and the layers separated. The aqueous layer was extracted with DCM (2 mL) and the combined organic extracts evaporated. The residue was purified by prep-HPLC (Method B1, 5-60% gradient) to give 5-(tert-butyl)-N-(2-fluoro-4-(6-(1-methyl-1H-pyrazol-4-yl)pyrazolo[1,5-a]pyrazin-4-yl)benzyl)-1,3,4-oxadiazole-2-carboxamide. LCMS m/z=475.1 [M+H]⁺. ¹H NMR (DMSO-d₆) δ: 9.91 (t, J=6.1 Hz, 1H), 9.18 (d, J=1.2 Hz, 1H), 8.37 (s, 1H), 8.21 (d, J=2.4 Hz, 1H), 8.13 (s, 1H), 7.98 (s, 1H), 7.88-7.97 (m, 1H), 7.62 (s, 1H), 7.21 (d, J=2.4 Hz, 1H), 4.62 (d, J=6.1 Hz, 2H), 3.91 (s, 3H), 1.40 (s, 9H).

### Example 7. 2-(tert-butyl)-N-(2-fluoro-4-(6-(1-methyl-1H-pyrazol-4-yl)pyrazolo[1,5-a]pyrazin-4-yl)benzyl)-2H-tetrazole-5-carboxamide

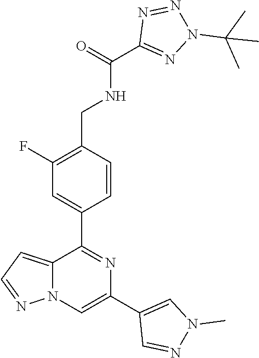 [SMILES: Cn1cc(-c2cn3nccc3c(-c3ccc(CNC(=O)c4nnn(C(C)(C)C)n4)c(F)c3)n2)cn1]

2-(tert-Butyl)-N-(2-fluoro-4-(6-(1-methyl-1H-pyrazol-4-yl)pyrazolo[1,5-a]pyrazin-4-yl)benzyl)-2H-tetrazole-5-carboxamide was obtained from 2-tert-butyltetrazolo-5-carboxylic acid and (2-fluoro-4-(6-(1-methyl-1H-pyrazol-4-yl)pyrazolo[1,5-a]pyrazin-4-yl)phenyl)methanamine dihydrochloride (Example 6, step 2) following a similar procedure to that described in Example 6, step 3. The crude product was purified by prep HPLC (Method B1, 5-60% gradient). LCMS m/z=475.1 [M+H]^{+ 1}H NMR (DMSO-d₆) δ: 9.66 (t, J=6.1 Hz, 1H), 9.18 (d, J=1.2 Hz, 1H), 8.37 (s, 1H), 8.25-8.20 (m, 1H), 8.13 (s, 1H), 7.99 (dd, J=7.9, 1.8 Hz, 1H), 7.94 (dd, J=11.0, 1.8 Hz, 1H), 7.64-7.55 (m, 1H), 7.25-7.19 (m, 1H), 4.65 (d, J=6.1 Hz, 2H), 3.91 (s, 3H), 1.74 (s, 9H).

### Example 8. N-(2-fluoro-4-(6-(1-methyl-1H-pyrazol-4-yl)pyrazolo[1,5-a]pyrazin-4-yl)benzyl)-5-(1-methylcyclopropyl)-1,2,4-oxadiazole-3-carboxamide

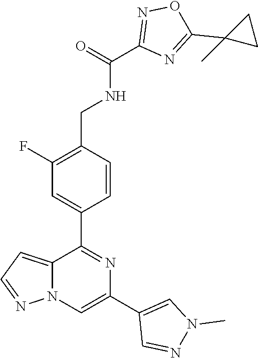 [SMILES: Cn1cc(-c2cn3nccc3c(-c3ccc(CNC(=O)c4noc(C5(C)CC5)n4)c(F)c3)n2)cn1]

N-(2-Fluoro-4-(6-(1-methyl-1H-pyrazol-4-yl)pyrazolo[1,5-a]pyrazin-4-yl)benzyl)-5-(1-methylcyclopropyl)-1,2,4-oxadiazole-3-carboxamide was prepared from (2-fluoro-4-(6-(1-methyl-1H-pyrazol-4-yl)pyrazolo[1,5-a]pyrazin-4-yl)phenyl)methanamine dihydrochloride (Example 6, step 2) and potassium 5-(1-methylcyclopropyl)-1,2,4-oxadiazole-3-carboxylate using a similar method to that used for Example 6, step 3. The crude material was purified by prep-HPLC (Method B1, gradient 5-60%). LCMS m/z=473.0 [M+H]⁺. ¹H NMR (DMSO-d₆) δ: 9.54 (t, J=5.8 Hz, 1H), 9.18 (s, 1H), 8.37 (s, 1H), 8.21 (d, J=2.4 Hz, 1H), 8.13 (s, 1H), 8.00 (dd, J=7.9, 1.8 Hz, 1H), 7.94 (dd, J=11.0, 1.2 Hz, 1H), 7.57 (t, J=7.9 Hz, 1H), 7.22 (d, J=2.4 Hz, 1H), 4.60 (d, J=6.1 Hz, 2H), 3.91 (s, 3H), 1.55 (s, 3H), 1.44-1.32 (m, 2H), 1.25-1.08 (m, 2H).

### Examples 9 to 13

The compounds in the following table were prepared from (2-fluoro-4-(6-(1-methyl-1H-pyrazol-4-yl)pyrazolo[1,5-a]pyrazin-4-yl)phenyl)methanamine dihydrochloride (Example 6, step 2) and the appropriate carboxylic acid, following a similar procedure to that described in Example 6, step 3.

| Example | |
|---|---|
| No. | Name/Structure/Carboxylic acid/HPLC/Data |
| 9 | 1-(tert-butyl)-N-(2-fluoro-4-(6-(1-methyl-1H-pyrazol-4-yl)pyrazolo[1,5- a]pyrazin-4-yl)benzyl)-1H-1,2,3-triazole-4-carboxamide 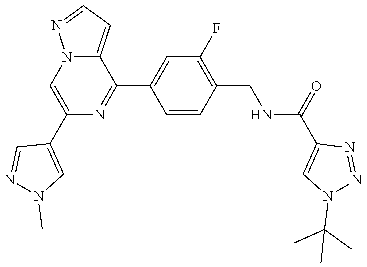 [SMILES: Cn1cc(-c2cn3nccc3c(-c3ccc(CNC(=O)c4cn(C(C)(C)C)nn4)c(F)c3)n2)cn1] Carboxylic Acid: 1-tert-butyltriazole-4-carboxylic acid Prep-HPLC (Method B1, 5-60% gradient) LCMS m/z = 474.1 [M + H]⁺. ¹H NMR (DMSO-d₆) δ: 9.24-9.11 (m, 2H), 8.72 (s, 1H), 8.37 (s, 1H), 8.20 (d, J = 2.4 Hz, 1H), 8.12 (s, 1H), 7.98 (d, J = 1.2 Hz, 1H), 7.93 (dd, J = 11.3, 1.5 Hz, 1H), 7.56 (s, 1H), 7.19-7.25 (m, 1H), 4.61 (d, J = 6.1 Hz, 2H), 3.91 (s, 3H), 1.64 (s, 9H). |
| 10 | 1-(tert-butyl)-N-(2-fluoro-4-(6-(1-methyl-1H-pyrazol-4-yl)pyrazolo[1,5- a]pyrazin-4-yl)benzyl)-1H-pyrazole-4-carboxamide 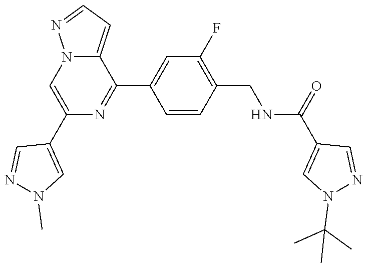 [SMILES: Cn1cc(-c2cn3nccc3c(-c3ccc(CNC(=O)c4cnn(C(C)(C)C)c4)c(F)c3)n2)cn1] Carboxylic acid: 1-tert-butylpyrazole-4-carboxylic acid Prep-HPLC (Method B1, 5-60% gradient) LCMS m/z = 473.1 [M + H]⁺. ¹H NMR (DMSO-d₆) δ: 9.17 (s, 1H), 8.66 (t, J = 5.8 Hz, 1H), 8.37 (s, 1H), 8.34 (s, 1H), 8.21 (d, J = 2.4 Hz, 1H), 8.13 (s, 1H), 7.99 (dd, J = 7.9, 1.8 Hz, 1H), 7.93 (s, 1H), 7.63-7.53 (m, 1H), 7.21 (d, J = 2.4 Hz, 1H), 4.56 (d, J = 6.1 Hz, 2H), 3.91 (s, 3H), 1.53 (s, 9H). |
| 11 | 1-(tert-butyl)-N-(2-fluoro-4-(6-(1-methyl-1H-pyrazol-4-yl)pyrazolo[1,5- a]pyrazin-4-yl)benzyl)-1H-pyrazole-3-carboxamide 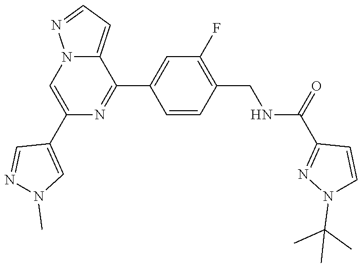 [SMILES: Cn1cc(-c2cn3nccc3c(-c3ccc(CNC(=O)c4ccn(C(C)(C)C)n4)c(F)c3)n2)cn1] Carboxylic Acid: 1-tert-butylpyrazole-3-carboxylic acid Prep-HPLC (Method B1, 5-60% gradient) LCMS m/z = 473.1 [M + H]⁺ ¹H NMR (DMSO-d₆) δ: 9.17 (s, 1H), 8.65 (t, J = 6.1 Hz, 1H), 8.37 (s, 1H), 8.20 (d, J = 2.4 Hz, 1H), 8.13 (s, 1H), 7.99 (dd, J = 8.2, 1.5 Hz, 1H), 7.96-7.86 (m, 2H), 7.56 (t, J = 7.9 Hz, 1H), 7.22 (d, J = 2.4 Hz, 1H), 6.67 (d, J = 2.4 Hz, 1H), 4.59 (d, J = 6.1 Hz, 2H), 3.91 (s, 3H), 1.57 (s, 9H). |
| 12 | 2-(tert-butyl)-N-(2-fluoro-4-(6-(1-methyl-1H-pyrazol-4-yl)pyrazolo[1,5- a]pyrazin-4-yl)benzyl)oxazole-4-carboxamide 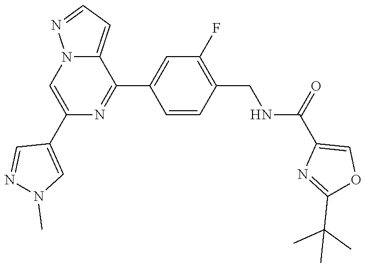 [SMILES: Cn1cc(-c2cn3nccc3c(-c3ccc(CNC(=O)c4coc(C(C)(C)C)n4)c(F)c3)n2)cn1] Carboxylic Acid: 2-tert-butyloxazole-4-carboxylic acid Prep-HPLC (Method B1, 5-60% gradient) LCMS m/z = 474.0 [M + H]⁺. ¹H NMR (DMSO-d₆) δ: 9.17 (s, 1H), 8.75 (t, J = 6.1 Hz, 1H), 8.56 (s, 1H), 8.37 (s, 1H), 8.20 (d, J = 2.4 Hz, 1H), 8.13 (s, 1H), 7.99 (dd, J = 7.9, 1.8 Hz, 1H), 7.93 (dd, J = 11.0, 1.2 Hz, 1H), 7.54 (t, J = 7.9 Hz, 1H), 7.21 (d, J = 1.8 Hz, 1H), 4.58 (d, J = 6.1 Hz, 2H), 3.91 (s, 3H), 1.48-1.27 (m, 9H). |
| 13 | 3-(1-fluoro-2-methylpropan-2-yl)-N-(2-fluoro-4-(6-(1-methyl-1H-pyrazol-4- yl)pyrazolo[1,5-a]pyrazin-4-yl)benzyl)-1,2,4-oxadiazole-5-carboxamide 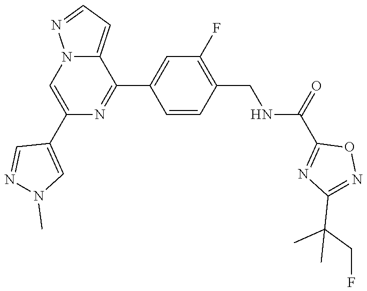 [SMILES: Cn1cc(-c2cn3nccc3c(-c3ccc(CNC(=O)c4nc(C(C)(C)CF)no4)c(F)c3)n2)cn1] Carboxylic acid: 3-(1-fluoro-2-methylpropan-2-yl)-1,2,4-oxadiazole-5- carboxylic acid (Example 71, step 6) Prep-HPLC (Method B1, 5-60% gradient) LCMS mz = 493.0 [M + H]⁺. ¹H NMR (DMSO-d₆) δ: 10.01 (t, J = 6.1 Hz, 1H), 9.18 (d, J = 1.2 Hz, 1H), 8.37 (s, 1H), 8.21 (d, J = 2.4 Hz, 1H), 8.13 (s, 1H), 8.00 (dd, J = 7.9, 1.8 Hz, 1H), 7.95 (dd, J = 11.3, 1.5 Hz, 1H), 7.63 (t, J = 7.9 Hz, 1H), 7.21 (dd, J = 2.4, 1.2 Hz, 1H), 4.71-4.57 (m, 3H), 4.53 (s, 1H), 3.91 (s, 2H), 1.38 (d, J = 1.8 Hz, 6H). |

### Example 14. 5-(tert-butyl)-N-(2-methyl-4-(6-(1-methyl-1H-pyrazol-4-yl)pyrazolo[1,5-a]pyrazin-4-yl)benzyl)-1,3,4-oxadiazole-2-carboxamide

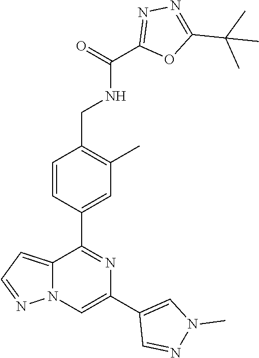 [SMILES: Cc1cc(-c2nc(-c3cnn(C)c3)cn3nccc23)ccc1CNC(=O)c1nnc(C(C)(C)C)o1]

### 1. Preparation of tert-butyl (2-methyl-4-(4,4,5,5-tetramethyl-1,3,2-dioxaborolan-2-yl)benzyl)carbamate

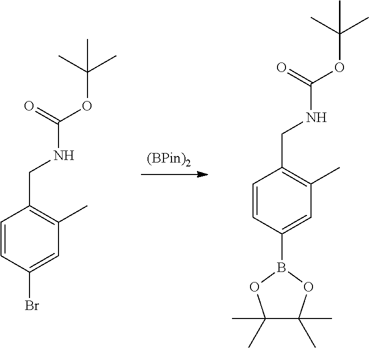 [SMILES: Cc1cc(Br)ccc1CNC(=O)OC(C)(C)C ; Cc1cc(B2OC(C)(C)C(C)(C)O2)ccc1CNC(=O)OC(C)(C)C]

To a solution of tert-butyl (4-bromo-2-methylbenzyl)carbamate (80 g, 266.50 mmol) and (BPin)₂ (81.21 g, 319.80 mmol) in dioxane (1 L) were added KOAc (78.46 g, 799.5 mmol) and Pd(dppf)Cl₂ (9.75 g, 13.32 mmol) and the mixture was stirred at 90° C. for 16 h under a N2 atmosphere. The reaction mixture was diluted with H₂O (600 mL) and extracted with EtOAc (3×500 mL). The combined organic layer was dried over Na₂SO₄ and filtered. The filtrate was concentrated in vacuo and the residue was purified by column chromatography on silica gel (PE/EtOAc=20/1) to give tert-butyl (2-methyl-4-(4,4,5,5-tetramethyl-1,3,2-dioxaborolan-2-yl)benzyl)carbamate (85 g, 91.9% yield) as a yellow solid. 1HNMR (400 MHz, CDCl₃) δ: 7.69-7.58 (m, 2H), 7.30-7.26 (m, 1H), 4.72 (br s, 1H), 4.36 (d, J=4.8 Hz, 2H), 2.35 (s, 3H), 1.48 (m, 9H), 1.38 (s, 12H). 2. Preparation of tert-butyl(2-methyl-4-(6-(1-methyl-1H-pyrazol-4-yl)pyrazolo[1,5-a]pyrazin-4-yl)benzyl)carbamate

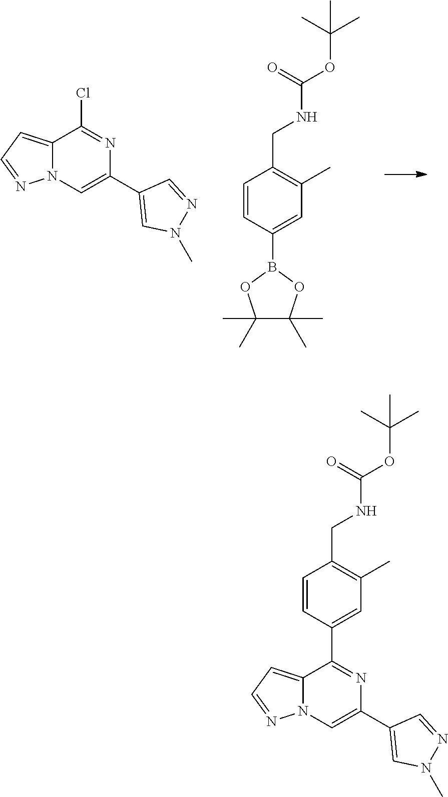 [SMILES: Cn1cc(-c2cn3nccc3c(Cl)n2)cn1 ; Cc1cc(B2OC(C)(C)C(C)(C)O2)ccc1CNC(=O)OC(C)(C)C ; Cc1cc(-c2nc(-c3cnn(C)c3)cn3nccc23)ccc1CNC(=O)OC(C)(C)C]

A mixture of 4-chloro-6-(1-methyl-1H-pyrazol-4-yl)pyrazolo[1,5-a]pyrazine (Example 1, step 5, 500 mg, 2.14 mmol), tert-butyl (2-methyl-4-(4,4,5,5-tetramethyl-1,3,2-dioxaborolan-2-yl)benzyl)carbamate (900 mg, 2.59 mmol), K₂CO₃ (700 mg, 5.06 mmol), and Pd(dppf)Cl₂ (30 mg, 41.0 μmol) was degassed under N2, then dioxane (10 mL) and water (2 mL) added and the reaction stirred at 90° C. in a sealed vessel for 20 mins. The reaction was removed from heating, allowed to cool to RT, then diluted with water (10 mL) and EtOAc (10 mL). The phases were allowed to separate, the aqueous layer was extracted with EtOAc (2×10 mL) and the organic layers combined and evaporated to dryness. The crude material was purified on silica gel (50% to 100% EtOAc in heptane) to give tert-butyl(2-methyl-4-(6-(1-methyl-1H-pyrazol-4-yl)pyrazolo[1,5-a]pyrazin-4-yl)benzyl)carbamate as a light yellow solid, (830 mg, 92.7% yield). LCMS m/z=419.3 [M+H]⁺

### 3. Preparation of 2-(tert-butyl)-N-(2-methyl-4-(5-(piperazin-1-yl)pyrimidin-4-yl)benzyl)-2H-tetrazole-5-carboxamide dihydrochloride

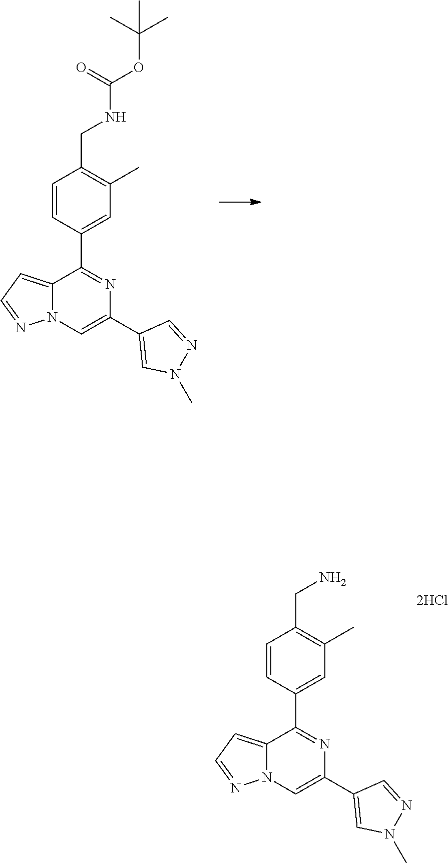 [SMILES: Cc1cc(-c2nc(-c3cnn(C)c3)cn3nccc23)ccc1CNC(=O)OC(C)(C)C ; Cc1cc(-c2nc(-c3cnn(C)c3)cn3nccc23)ccc1CN]

A solution of tert-butyl(2-methyl-4-(6-(1-methyl-1H-pyrazol-4-yl)pyrazolo[1,5-a]pyrazin-4-yl)benzyl)carbamate (830 mg, 1.98 mmol) in 1.25M methanolic HCl (16 mL) was stirred at RT for 16 h. The mixture was heated to vigorous reflux for 1 h, then concentrated in vacuo to give 2-(tert-butyl)-N-(2-methyl-4-(5-(piperazin-1-yl)pyrimidin-4-yl)benzyl)-2H-tetrazole-5-carboxamide dihydrochloride as a light yellow solid. LCMS m/z=319.2 [M+H]⁺

### 4. Preparation of 5-(tert-butyl)-N-(2-methyl-4-(6-(1-methyl-1H-pyrazol-4-yl)pyrazolo[1,5-a]pyrazin-4-yl)benzyl)-1,3,4-oxadiazole-2-carboxamide

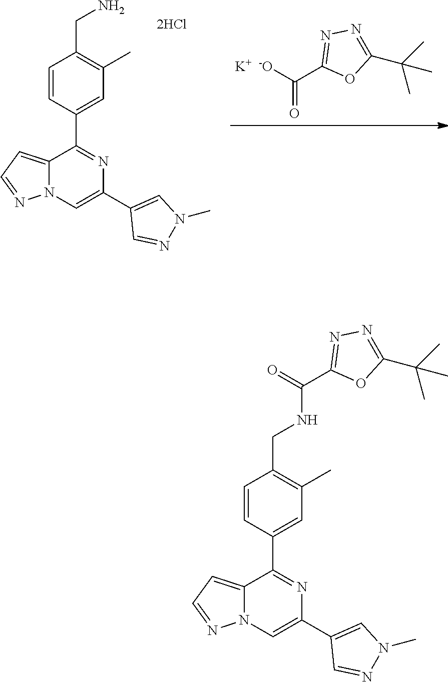

SUBSTRATE STOCK SOLUTION—To an 8 mL vial was added 2-(tert-butyl)-N-(2-methyl-4-(5-(piperazin-1-yl)pyrimidin-4-yl)benzyl)-2H-tetrazole-5-carboxamide dihydrochloride (175 mg) and DIPEA (250 μL) in DMF (2.5 mL) and the mixture stirred for 10 mins.

To a vial containing potassium 5-tert-butyl-1,3,4-oxadiazole-2-carboxylate (20 mg, 96.03 μmol) was added 600 μL of the substrate stock solution followed by T3P® (125.0 mg, 196.43 μmol, 50% purity). and the reaction stirred at RT for 2 h. The reaction was quenched by the addition of satd. NaHCO₃ (3 mL) and extracted with EtOAc (3 mL). The resulting aqueous slurry was extracted further with EtOAc (3×3 mL) and the combined organic layers were concentrated to dryness. The residue was diluted with DMSO (2 mL) and passed through a 0.2 um syringe filter. The product was isolated by prep-HPLC (Method B1, 5-60% gradient). LCMS m/z=471.2 [M+H]^{+ 1}H NMR (500 MHz, DMSO-d₆) δ ppm 9.85 (t, J=6.10 Hz, 1H) 9.13 (s, 1H) 8.34 (s, 1H) 8.19 (d, J=2.44 Hz, 1H) 8.12 (s, 1H) 7.95 (s, 2H) 7.50 (s, 1H) 7.15 (d, J=2.44 Hz, 1H) 4.56 (d, J=6.10 Hz, 2H) 3.92 (s, 3H) 2.49 (s, 3H) 1.41 (s, 9H).

### Example 15. 2-(tert-butyl)-N-(2-methyl-4-(6-(1-methyl-1H-pyrazol-4-yl)pyrazolo[1,5-a]pyrazin-4-yl)benzyl)-2H-tetrazole-5-carboxamide

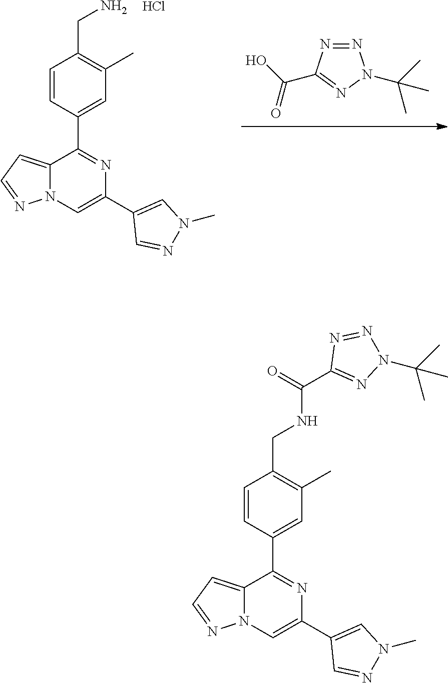 [SMILES: Cc1cc(-c2nc(-c3cnn(C)c3)cn3nccc23)ccc1CN ; Cl ; CC(C)(C)n1nnc(C(=O)O)n1 ; Cc1cc(-c2nc(-c3cnn(C)c3)cn3nccc23)ccc1CNC(=O)c1nnn(C(C)(C)C)n1]

2-(tert-Butyl)-N-(2-methyl-4-(6-(1-methyl-1H-pyrazol-4-yl)pyrazolo[1,5-a]pyrazin-4-yl)benzyl)-2H-tetrazole-5-carboxamide was prepared from 2-tert-butyl-2H-1,2,3,4-tetrazole-5-carboxylic acid and 2-(tert-butyl)-N-(2-methyl-4-(5-(piperazin-1-yl)pyrimidin-4-yl)benzyl)-2H-tetrazole-5-carboxamide dihydrochloride (Example 14, step 3) following the method used in Example 14, step 4. LCMS m/z=471.3 [M+H]⁺. ¹H NMR (500 MHz, DMSO-d₆) δ ppm 9.58 (s, 1H) 9.13 (s, 1H) 8.34 (s, 1H) 8.19 (d, J=2.44 Hz, 1H) 8.12 (s, 1H) 7.95 (s, 2H) 7.51-7.43 (m, 1H) 7.18-7.12 (m, 1H) 4.59 (d, J=6.10 Hz, 2H) 3.91 (s, 3H) 2.50-2.48 (s, 3H) 1.75 (s, 9H).

### Example 16. 1-(tert-butyl)-N-(2-methyl-4-(6-(1-methyl-1H-pyrazol-4-yl)pyrazolo[1,5-a]pyrazin-4-yl)benzyl)-1H-1,2,3-triazole-4-carboxamide

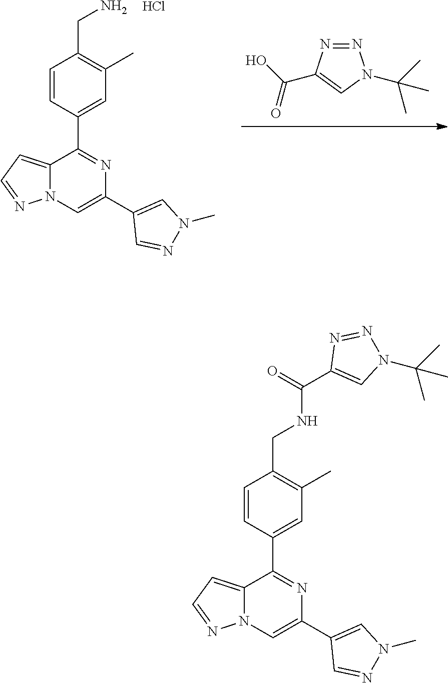 [SMILES: Cc1cc(-c2nc(-c3cnn(C)c3)cn3nccc23)ccc1CN ; Cl ; CC(C)(C)n1cc(C(=O)O)nn1 ; Cc1cc(-c2nc(-c3cnn(C)c3)cn3nccc23)ccc1CNC(=O)c1cn(C(C)(C)C)nn1]

1-(tert-Butyl)-N-(2-methyl-4-(6-(1-methyl-1H-pyrazol-4-yl)pyrazolo[1,5-a]pyrazin-4-yl)benzyl)-1H-1,2,3-triazole-4-carboxamide was prepared from 1-tert-butyltriazole-4-carboxylic acid and 2-(tert-butyl)-N-(2-methyl-4-(5-(piperazin-1-yl)pyrimidin-4-yl)benzyl)-2H-tetrazole-5-carboxamide dihydrochloride (Example 14, step 3) following the method used in Example 14, step 4. LCMS m/z=470.2 [M+H]⁺. ¹H NMR (500 MHz, DMSO-d₆) δ ppm 9.12 (s, 1H) 9.07 (s, 1H) 8.73 (s, 1H) 8.34 (s, 1H) 8.18 (d, J=2.44 Hz, 1H) 8.12 (s, 1H) 7.94 (s, 2H) 7.50-7.40 (m, 1H) 7.16 (d, J=2.44 Hz, 1H) 4.55 (d, J=6.10 Hz, 2H) 3.91 (s, 3H) 2.49-2.47 (m, 3H) 1.65 (s, 9H).

### Example 17. 1-(tert-butyl)-N-(2-methyl-4-(6-(1-methyl-1H-pyrazol-4-yl)pyrazolo[1,5-a]pyrazin-4-yl)benzyl)-1H-pyrazole-4-carboxamide

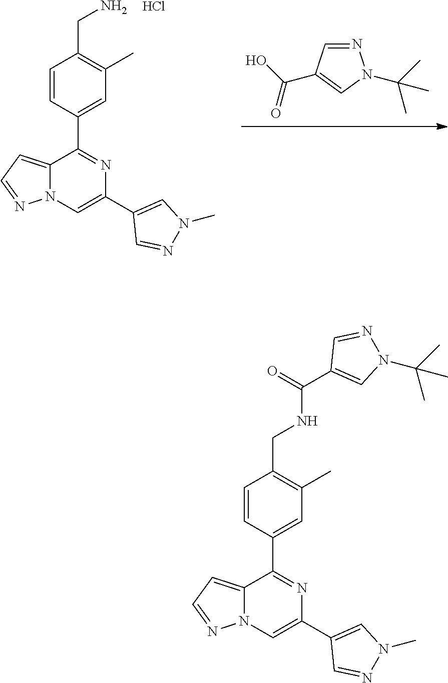 [SMILES: Cc1cc(-c2nc(-c3cnn(C)c3)cn3nccc23)ccc1CN ; Cl ; CC(C)(C)n1cc(C(=O)O)cn1 ; Cc1cc(-c2nc(-c3cnn(C)c3)cn3nccc23)ccc1CNC(=O)c1cnn(C(C)(C)C)c1]

1-(tert-Butyl)-N-(2-methyl-4-(6-(1-methyl-1H-pyrazol-4-yl)pyrazolo[1,5-a]pyrazin-4-yl)benzyl)-1H-pyrazole-4-carboxamide was prepared from 1-tert-butylpyrazole-4-carboxylic acid and 2-(tert-butyl)-N-(2-methyl-4-(5-(piperazin-1-yl)pyrimidin-4-yl)benzyl)-2H-tetrazole-5-carboxamide dihydrochloride (Example 14, step 3) following the method used in Example 14, step 4. LCMS m/z=469.3 [M+H]⁺. ¹H NMR (500 MHz, DMSO-d₆) δ ppm 9.13 (d, J=1.22 Hz, 1H) 8.58-8.47 (m, 2H) 8.35 (d, J=5.49 Hz, 2H) 8.19 (d, J=2.44 Hz, 1H) 8.12 (s, 1H) 7.94 (s, 4H) 7.45 (d, J=8.55 Hz, 1H) 7.15 (d, J=2.44 Hz, 1H) 4.51 (d, J=5.49 Hz, 2H) 3.92 (s, 3H) 2.47 (s, 3H) 1.59-1.48 (m, 9H).

### Example 18. 1-(tert-butyl)-N-(2-methyl-4-(6-(1-methyl-1H-pyrazol-4-yl)pyrazolo[1,5-a]pyrazin-4-yl)benzyl)-1H-pyrazole-3-carboxamide

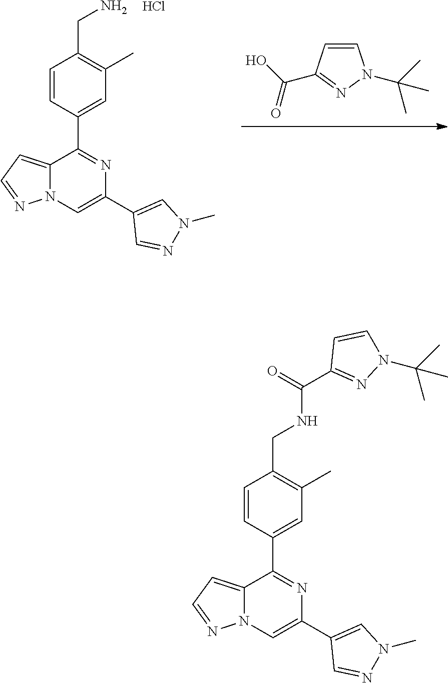 [SMILES: Cc1cc(-c2nc(-c3cnn(C)c3)cn3nccc23)ccc1CN ; Cl ; CC(C)(C)n1ccc(C(=O)O)n1 ; Cc1cc(-c2nc(-c3cnn(C)c3)cn3nccc23)ccc1CNC(=O)c1ccn(C(C)(C)C)n1]

1-(tert-Butyl)-N-(2-methyl-4-(6-(1-methyl-1H-pyrazol-4-yl)pyrazolo[1,5-a]pyrazin-4-yl)benzyl)-1H-pyrazole-3-carboxamide was prepared from 1-tert-butylpyrazole-3-carboxylic acid and 2-(tert-butyl)-N-(2-methyl-4-(5-(piperazin-1-yl)pyrimidin-4-yl)benzyl)-2H-tetrazole-5-carboxamide dihydrochloride (Example 14, step 3) following the method used in Example 14, step 4. LCMS m/z=469.3 [M+H]⁺. ¹H NMR (500 MHz, DMSO-d₆) δ ppm 9.12 (d, J=1.22 Hz, 1H) 8.54 (t, J=6.10 Hz, 1H) 8.34 (s, 1H) 8.18 (d, J=2.44 Hz, 1H) 8.12 (s, 1H) 8.01-7.86 (m, 2H) 7.49-7.40 (m, 1H) 7.16 (s, 1H) 6.72-6.62 (m, 1H) 4.53 (d, J=6.10 Hz, 2H) 3.91 (s, 3H) 2.48 (s, 3H) 1.58 (s, 9H).

### Example 19: Synthesis of 3-(tert-butyl)-N-(4-(6-(1-methyl-1H-pyrazol-4-yl)pyrazolo[1,5-a]pyrazin-4-yl)benzyl)-1,2,4-oxadiazole-5-carboxamide

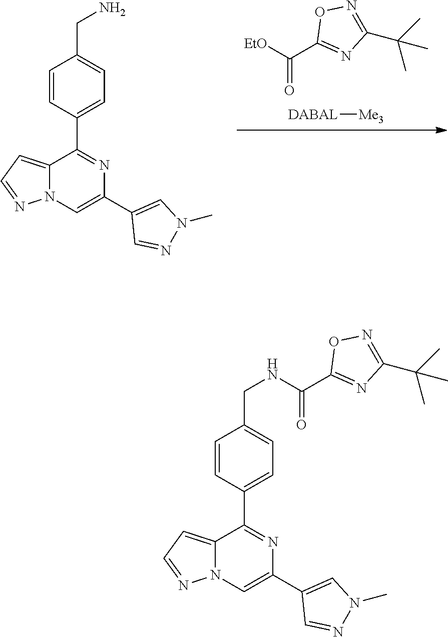

To a solution of (4-(6-(1-methyl-1H-pyrazol-4-yl)pyrazolo[1,5-a]pyrazin-4-yl)phenyl)methanamine dihydrochloride (Example 1, step 7, 30 mg, 99 μmol) and ethyl 3-(tert-butyl)-1,2,4-oxadiazole-5-carboxylate (29 mg, 148 μmol) in THF (1.5 mL) was added DABAL-Me₃ (38 mg, 148 μmol) and the reaction was heated to 45° C. and stirred at that temperature for 18 h. The reaction mixture was cooled to ambient temperature and diluted with water (1 mL), 1M HCl solution (1 mL) and EtOAc (5 mL). The layers were separated, and the aqueous phase was extracted with additional portions of EtOAc. The combined organic phase was washed with brine, dried (Na₂SO₄), filtered and concentrated. The crude material was purified by reverse phase HPLC (Method A, 5-65% gradient) to give 3-(tert-butyl)-N-(4-(6-(1-methyl-1H-pyrazol-4-yl)pyrazolo[1,5-a]pyrazin-4-yl)benzyl)-1,2,4-oxadiazole-5-carboxamide as a beige solid (4.7 mg, 10% yield). LCMS m/z=457.0 [M+H]⁺. ¹H NMR (500 MHz, DMSO-d₆) δ: 9.98 (t, J=6.2 Hz, 1H), 9.13 (s, 1H), 8.34 (s, 1H), 8.19 (d, J=2.4 Hz, 1H), 8.14-8.11 (m, 3H), 7.57 (d, J=7.9 Hz, 2H), 7.16 (s, 1H), 4.58 (d, J=6.1 Hz, 2H), 3.90 (s, 3H), 1.37 (s, 9H).

### Example 20: Synthesis of 5-(1-(fluoromethyl)cyclopropyl)-N-(4-(6-(1-methyl-1H-pyrazol-4-yl)pyrazolo[1,5-a]pyrazin-4-yl)benzyl)-1,2,4-oxadiazole-3-carboxamide

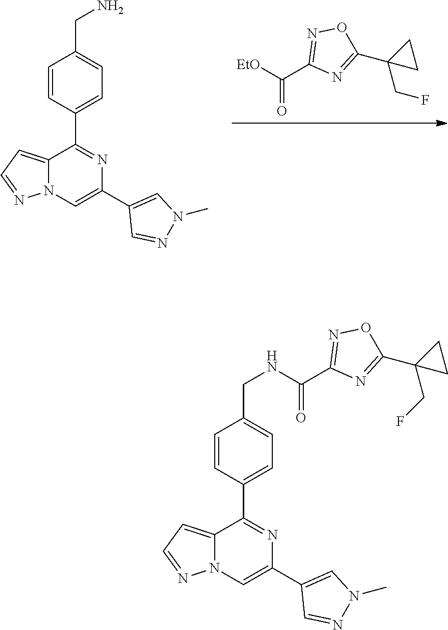 [SMILES: Cn1cc(-c2cn3nccc3c(-c3ccc(CN)cc3)n2)cn1 ; CCOC(=O)c1noc(C2(CF)CC2)n1 ; Cn1cc(-c2cn3nccc3c(-c3ccc(CNC(=O)c4noc(C5(CF)CC5)n4)cc3)n2)cn1]

5-(1-(Fluoromethyl)cyclopropyl)-N-(4-(6-(1-methyl-1H-pyrazol-4-yl)pyrazolo[1,5-a]pyrazin-4-yl)benzyl)-1,2,4-oxadiazole-3-carboxamide was obtained (29.6 mg, 47% yield) from (4-(6-(1-methyl-1H-pyrazol-4-yl)pyrazolo[1,5-a]pyrazin-4-yl)phenyl)methanamine dihydrochloride (Example 1, step 7) and ethyl 5-(1-(fluoromethyl)cyclopropyl)-1,2,4-oxadiazole-3-carboxylate following a similar method to that of Example 19. The crude material was purified by reverse phase HPLC (Method C, 5-55% gradient). LCMS m/z=473.1 [M+H]^{+ 1}H NMR (500 MHz, DMSO-d₆) δ: 9.60 (t, J=6.4 Hz, 1H), 9.13 (s, 1H), 8.34 (s, 1H), 8.19 (d, J=2.4 Hz, 1H), 8.16-8.08 (m, 3H), 7.54 (d, J=7.9 Hz, 2H), 7.20-7.13 (m, 1H), 4.83 (s, 1H), 4.73 (s, 1H), 4.57 (d, J=6.1 Hz, 2H), 3.90 (s, 3H), 1.60-1.52 (m, 2H), 1.51-1.44 (m, 2H).

### Example 21: Synthesis of 2-(tert-butyl)-N-(4-(6-(1-methyl-1H-pyrazol-4-yl)pyrazolo[1,5-a]pyrazin-4-yl)benzyl)-2H-1,2,3-triazole-4-carboxamide

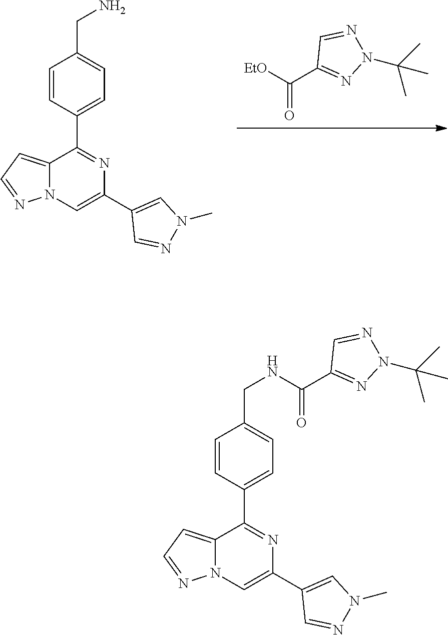 [SMILES: Cn1cc(-c2cn3nccc3c(-c3ccc(CN)cc3)n2)cn1 ; CCOC(=O)c1cnn(C(C)(C)C)n1 ; Cn1cc(-c2cn3nccc3c(-c3ccc(CNC(=O)c4cnn(C(C)(C)C)n4)cc3)n2)cn1]

2-(tert-Butyl)-N-(4-(6-(1-methyl-1H-pyrazol-4-yl)pyrazolo[1,5-a]pyrazin-4-yl)benzyl)-2H-1,2,3-triazole-4-carboxamide was obtained (23.8 mg, 39% yield) from (4-(6-(1-methyl-1H-pyrazol-4-yl)pyrazolo[1,5-a]pyrazin-4-yl)phenyl)methanamine dihydrochloride (Example 1, step 7) and ethyl 2-(tert-butyl)-2H-1,2,3-triazole-4-carboxylate (Journal of Medicinal Chemistry (2018), 61(8), 3370-3388), following a similar method to that of Example 19. The crude material was purified by reverse phase HPLC (Method C, 5-60% gradient). LCMS m/z=456.2 [M+H]⁺. ¹H NMR (500 MHz, DMSO-d₆) δ: 9.13 (s, 1H), 9.05 (t, J=6.1 Hz, 1H), 8.33 (s, 1H), 8.18 (d, J=2.4 Hz, 1H), 8.16 (s, 1H), 8.14-8.10 (m, 3H), 7.55 (d, J=7.9 Hz, 2H), 7.19-7.14 (m, 1H), 4.57 (d, J=6.1 Hz, 2H), 3.90 (s, 3H), 1.69-1.62 (m, 9H).

### Example 22: 5-(1-fluoro-2-methylpropan-2-yl)-N-(4-(6-(1-methyl-1H-pyrazol-4-yl)pyrazolo[1,5-a]pyrazin-4-yl)benzyl)-1,2,4-oxadiazole-3-carboxamide

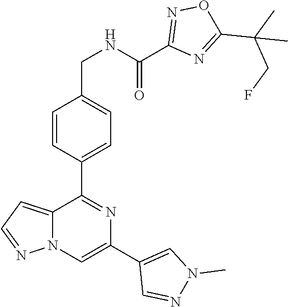 [SMILES: Cn1cc(-c2cn3nccc3c(-c3ccc(CNC(=O)c4noc(C(C)(C)CF)n4)cc3)n2)cn1]

### 1. Synthesis of ethyl (E)-2-amino-2-(hydroxyimino)acetate

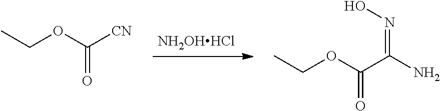

To a solution of ethyl carbonocyanidate (10 g, 101 mmol) in EtOH (75 mL) and H₂O (25 mL) was added Na₂CO₃ (10.7 g, 101 mmol) and hydroxylamine hydrochloride (7.0 g, 101 mmol) and the reaction mixture was stirred at 25° C. for 12 h. The mixture was poured into EtOAc (100 mL), filtered, and the filtrate was concentrated in vacuo. The residue was re-dissolved in EtOAc (100 mL), filtered, and the filtrate was concentrated in vacuo to give ethyl (E)-2-amino-2-(hydroxyimino)acetate as a yellow solid (10 g, crude), which was carried forward without further purification. ¹H NMR (400 MHz, CDCl₃) δ: 4.37-4.32 (m, 2H), 1.38-1.30 (m, 3H).

### 2. Synthesis of ethyl (E)-2-(3-hydroxy-2,2-dimethylpropanamido)-2-(hydroxyimino)acetate

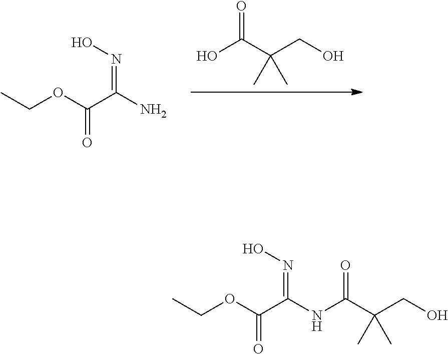

A mixture of ethyl (E)-2-amino-2-(hydroxyimino)acetate (500 mg, 3.8 mmol), 3-hydroxy-2,2-dimethylpropanoic acid (447 mg, 3.8 mmol), HATU (2.9 g, 7.6 mmol), and DIPEA (977 mg, 7.6 mmol) in DCM (10 mL) was stirred at 25° C. for 1 h. An additional volume of DCM (30 mL) was added and the organic phase was washed with H₂O (10 mL×3). The organic phase was dried (Na₂SO₄), filtered, and concentrated in vacuo. The crude product was purified by silica-gel column chromatography (PE/EtOAc, 5:1 to 0:1) to give ethyl (E)-2-(3-hydroxy-2,2-dimethylpropanamido)-2-(hydroxyimino)acetate as a white solid (500 mg, yield: 57%). LCMS m/z=233.1 [M+H]⁺.

### 3. Synthesis of ethyl 5-(1-hydroxy-2-methylpropan-2-yl)-1,2,4-oxadiazole-3-carboxylate

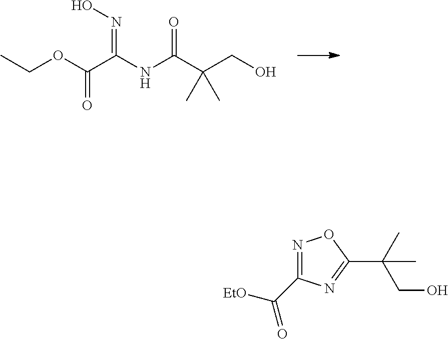 [SMILES: CCOC(=O)/C(=N\O)NC(=O)C(C)(C)CO ; CCOC(=O)c1noc(C(C)(C)CO)n1]

A solution of ethyl (E)-2-(3-hydroxy-2,2-dimethylpropanamido)-2-(hydroxyimino)acetate (500 mg, 2.2 mmol) in pyridine (10 mL) was heated to 80° C. and was stirred at that temperature for 12 h. The reaction mixture was concentrated in vacuo and the residue was purified by silica-gel column chromatography (PE/EtOAc, 10:1 to 1:1) to give ethyl 5-(1-hydroxy-2-methylpropan-2-yl)-1,2,4-oxadiazole-3-carboxylate as a colorless oil (250 mg, yield: 54%). LCMS m/z=215.1 [M+H]⁺.

### 4. Synthesis of ethyl 5-(2-methyl-1-(((trifluoromethyl)sulfonyl)oxy)propan-2-yl)-1,2,4-oxadiazole-3-carboxylate

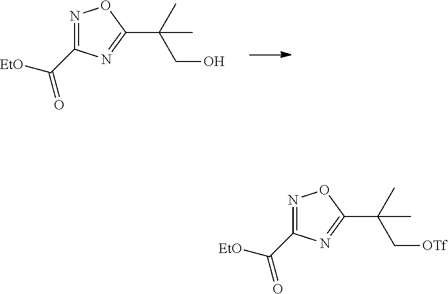 [SMILES: CCOC(=O)c1noc(C(C)(C)CO)n1 ; CCOC(=O)c1noc(C(C)(C)COS(=O)(=O)C(F)(F)F)n1]

To a solution of ethyl 5-(1-hydroxy-2-methylpropan-2-yl)-1,2,4-oxadiazole-3-carboxylate (430 mg, 2.0 mmol) and DIPEA (390 mg, 3.0 mmol) in DCM (10 mL) at 0° C. was added Tf₂O (566 mg, 2.0 eq) and the reaction mixture was stirred at 25° C. for 2 h. An additional volume of DCM (40 mL) was added and the organic phase was washed with H₂O (20 mL×3). The organic phase was dried (Na₂SO₄), filtered, and concentrated in vacuo. The crude product was purified by silica-gel column chromatography (PE/EtOAc, 10:1 to 1:1) to give ethyl 5-(2-methyl-1-(((trifluoromethyl)sulfonyl)oxy)propan-2-yl)-1,2,4-oxadiazole-3-carboxylate as a colorless oil (600 mg, yield: 86%). LCMS m/z=347.1 [M+H]⁺.

### 5. Synthesis of ethyl 5-(1-fluoro-2-methylpropan-2-yl)-1,2,4-oxadiazole-3-carboxylate

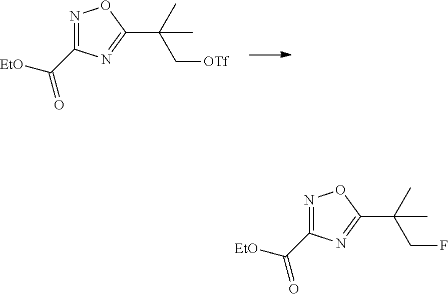 [SMILES: CCOC(=O)c1noc(C(C)(C)COS(=O)(=O)C(F)(F)F)n1 ; CCOC(=O)c1noc(C(C)(C)CF)n1]

A solution of ethyl 5-(2-methyl-1-(((trifluoromethyl)sulfonyl)oxy)propan-2-yl)-1,2,4-oxadiazole-3-carboxylate (600 mg, 1.7 mmol) and tetrabutylammonium bifluoride (536 mg, 1.9 mmol) in THF (30 mL) was heated to 40° C. and stirred at that temperature for 12 h. The reaction mixture was cooled to ambient temperature and was concentrated in vacuo. The crude product was purified by silica-gel column chromatography (PE/EtOAc, 10:1 to 3:1) to give ethyl 5-(1-fluoro-2-methylpropan-2-yl)-1,2,4-oxadiazole-3-carboxylate as a colorless oil (330 mg, yield: 88%). LCMS m/z=217.1 [M+H]⁺.

### 6. Synthesis of 5-(1-fluoro-2-methylpropan-2-yl)-N-(4-(6-(1-methyl-1H-pyrazol-4-yl)pyrazolo[1,5-a]pyrazin-4-yl)benzyl)-1,2,4-oxadiazole-3-carboxamide

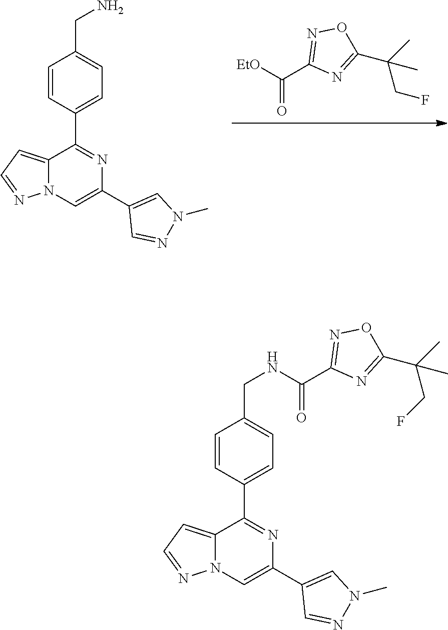 [SMILES: Cn1cc(-c2cn3nccc3c(-c3ccc(CN)cc3)n2)cn1 ; CCOC(=O)c1noc(C(C)(C)CF)n1 ; Cn1cc(-c2cn3nccc3c(-c3ccc(CNC(=O)c4noc(C(C)(C)CF)n4)cc3)n2)cn1]

5-(1-Fluoro-2-methylpropan-2-yl)-N-(4-(6-(1-methyl-1H-pyrazol-4-yl)pyrazolo[1,5-a]pyrazin-4-yl)benzyl)-1,2,4-oxadiazole-3-carboxamide was obtained (37.6 mg, 60% yield) from ethyl 5-(1-fluoro-2-methylpropan-2-yl)-1,2,4-oxadiazole-3-carboxylate and (4-(6-(1-methyl-1H-pyrazol-4-yl)pyrazolo[1,5-a]pyrazin-4-yl)phenyl)methanamine dihydrochloride (Example 1, step 7) following a similar method to that of Example 19. The crude material was purified by reverse phase HPLC (Method C, gradient 5-55%). LCMS m/z=475.2 [M+H]⁺. ¹H NMR (500 MHz, DMSO-d₆) δ=9.65 (t, J=6.1 Hz, 1H), 9.13 (d, J=1.2 Hz, 1H), 8.34 (s, 1H), 8.19 (d, J=2.4 Hz, 1H), 8.16-8.08 (m, 3H), 7.55 (d, J=8.5 Hz, 2H), 7.19-7.13 (m, 1H), 4.68 (s, 1H), 4.63-4.53 (m, 4H), 3.90 (s, 3H), 1.45 (d, J=1.2 Hz, 9H)

### Example 23. 5-(tert-butyl)-N-(4-(6-morpholinopyrazolo[1,5-a]pyrazin-4-yl)benzyl)-1,2,4-oxadiazole-3-carboxamide

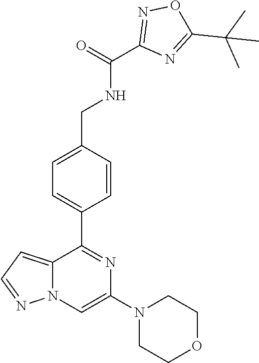 [SMILES: CC(C)(C)c1nc(C(=O)NCc2ccc(-c3nc(N4CCOCC4)cn4nccc34)cc2)no1]

### 1. Preparation of tert-butyl (4-(6-chloropyrazolo[1,5-a]pyrazin-4-yl)benzyl)carbamate

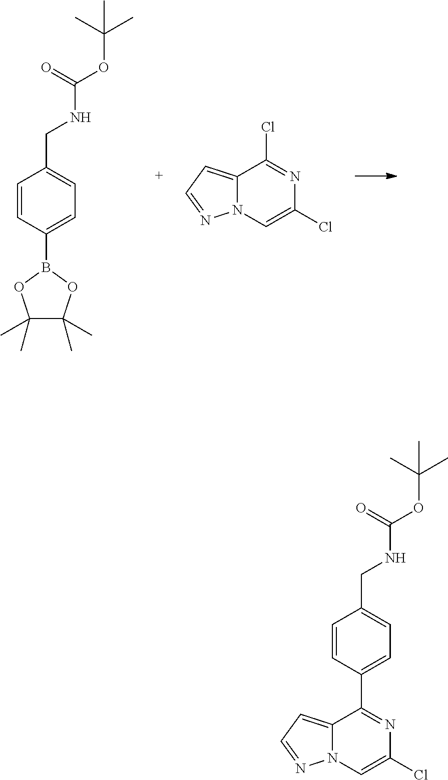 [SMILES: CC(C)(C)OC(=O)NCc1ccc(B2OC(C)(C)C(C)(C)O2)cc1 ; Clc1cn2nccc2c(Cl)n1 ; CC(C)(C)OC(=O)NCc1ccc(-c2nc(Cl)cn3nccc23)cc1]

A mixture of tert-butyl (4-(4,4,5,5-tetramethyl-1,3,2-dioxaborolan-2-yl)benzyl)carbamate (350 mg, 1.05 mmol), 4,6-dichloropyrazolo[1,5-a]pyrazine (200 mg, 1.06 mmol), K₃PO₄ (2.0 M, 824.4 uL) and dioxane (5.0 mL) was stirred at RT and degassed for 10 mins by bubbling N2 through. Pd(t-Bu₃P)₂ (15 mg, 29.35 μmol) was added, the vial sealed, and the reaction stirred at 40° C. for 72 h. The reaction was diluted with water (10 mL) and extracted with EtOAc (3×10 mL). The combined organic layers were concentrated to dryness and purified by silica gel column chromatography (heptanes to EtOAc) to give tert-butyl (4-(6-chloropyrazolo[1,5-a]pyrazin-4-yl)benzyl)carbamate as a light yellow solid (290 mg, 76.3% yield). LCMS m/z=359.1 [M+H]⁺. 2. Preparation of tert-butyl (4-(6-morpholinopyrazolo[1,5-a]pyrazin-4-yl)benzyl)carbamate

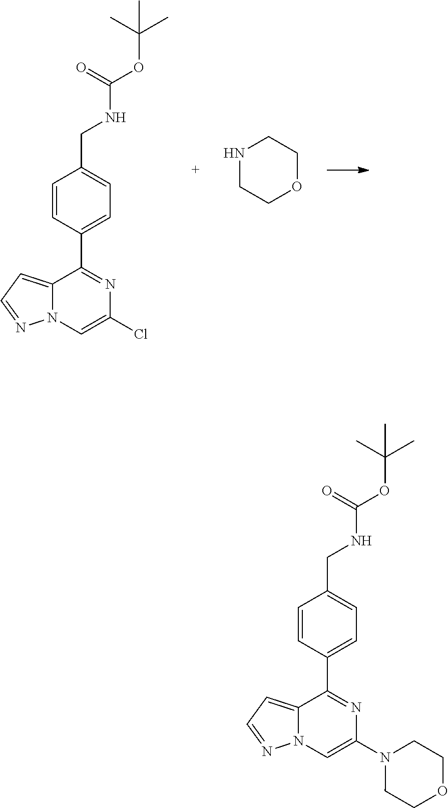 [SMILES: CC(C)(C)OC(=O)NCc1ccc(-c2nc(Cl)cn3nccc23)cc1 ; C1COCCN1 ; CC(C)(C)OC(=O)NCc1ccc(-c2nc(N3CCOCC3)cn3nccc23)cc1]

To a vial containing tert-butyl (4-(6-chloropyrazolo[1,5-a]pyrazin-4-yl)benzyl)carbamate (160 mg, 445.9 μmol) was added Pd₂(dba)₃ (20 mg, 21.8 μmol), Cs₂CO₃ (450 mg, 1.38 mmol) and RuPhos (20 mg, 42.9 μmol), the vial sealed and degassed under N2. Dioxane (5.04 mL) and morpholine (65 mg, 746.1 μmol) were added and the reaction stirred at 100° C. for 4 h. The cooled reaction was diluted with water (10 mL), extracted with EtOAc (3×10 mL) and the combined organic layers were concentrated to dryness to give a bright orange solid. This was purified by silica gel chromatography (heptanes to EtOAc) to give tert-butyl (4-(6-morpholinopyrazolo[1,5-a]pyrazin-4-yl)benzyl)carbamate as a light yellow solid (150 mg, 82.2% yield). LCMS m/z=410.2 [M+H]⁺

### 3. Preparation of (4-(6-morpholinopyrazolo[1,5-a]pyrazin-4-yl)phenyl)methanamine dihydrochloride

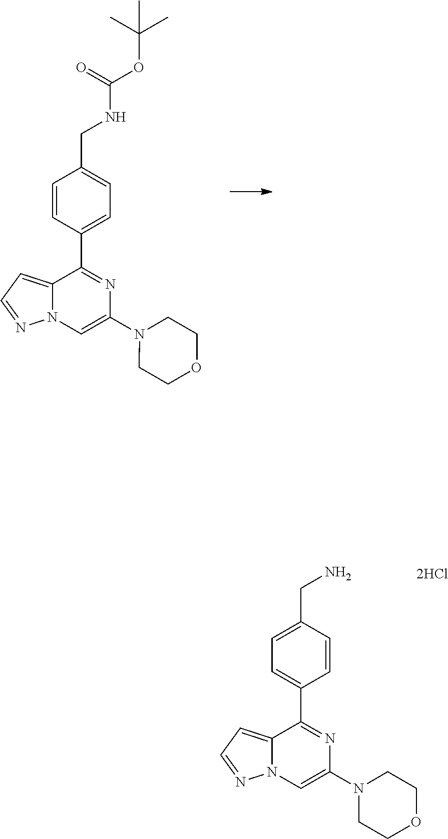 [SMILES: CC(C)(C)OC(=O)NCc1ccc(-c2nc(N3CCOCC3)cn3nccc23)cc1 ; NCc1ccc(-c2nc(N3CCOCC3)cn3nccc23)cc1]

A solution of tert-butyl (4-(6-morpholinopyrazolo[1,5-a]pyrazin-4-yl)benzyl)carbamate (150 mg, 366.3 μmol) and 4M HCl (500 uL) in dioxane (5.00 mL) was stirred at 50° C. for 16 h. The reaction mixture was concentrated in vacuo and azeotroped with MeOH to give (4-(6-morpholinopyrazolo[1,5-a]pyrazin-4-yl)phenyl)methanamine dihydrochloride as a tan solid (148 mg, crude). LCMS m/z=310.1 [M+H]⁺

### 4. Preparation of 5-(tert-butyl)-N-(4-(6-morpholinopyrazolo[1,5-a]pyrazin-4-yl)benzyl)-1,2,4-oxadiazole-3-carboxamide

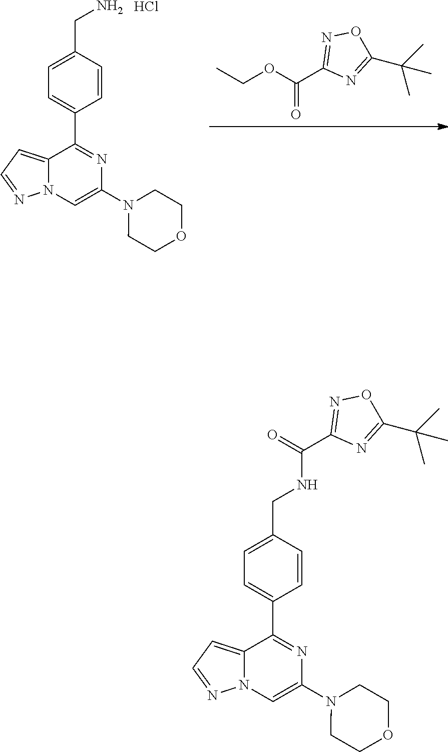 [SMILES: NCc1ccc(-c2nc(N3CCOCC3)cn3nccc23)cc1 ; Cl ; CCOC(=O)c1noc(C(C)(C)C)n1 ; CC(C)(C)c1nc(C(=O)NCc2ccc(-c3nc(N4CCOCC4)cn4nccc34)cc2)no1]

To a vial was added (4-(6-morpholinopyrazolo[1,5-a]pyrazin-4-yl)phenyl)methanamine hydrochloride (20 mg, 57.8 μmol), DABAL-Me₃ (15 mg, 58.5 μmol) and ethyl 5-tert-butyl-1,2,4-oxadiazole-3-carboxylate (20 mg, 100.9 μmol) in dioxane (500 uL), the vial sealed and stirred at 120° C. for 10 min. Celite® (250 mg) was added to the cooled mixture, followed by water (0.5 mL) and EtOH (2 mL) and the mixture stirred for 15 mins. The slurry was centrifuged and mother liquor decanted. The remaining solids were extracted with EtOH (2×5 mL), repeating centrifugation and mother liquor collection. The combined mother liquors were concentrated to dryness to give a bright yellow solid. This was purified by prep-HPLC (Method C2, 5-60% gradient) to provide 5-(tert-butyl)-N-(4-(6-morpholinopyrazolo[1,5-a]pyrazin-4-yl)benzyl)-1,2,4-oxadiazole-3-carboxamide. LCMS m/z=462.2 [M+H]^{+ 1}H NMR (500 MHz, DMSO-d₆) δ ppm 9.59 (br t, J=6.10 Hz, 1H) 8.16 (s, 1H) 8.11-7.98 (m, 2H) 7.53 (d, J=8.55 Hz, 1H) 7.12-7.02 (m, 1H) 4.65-4.45 (m, 2H) 3.87-3.70 (m, 3H) 1.44 (s, 9H).

### Example 24: 3-(tert-butyl)-N-(4-(6-morpholinopyrazolo[1,5-a]pyrazin-4-yl)benzyl)-1,2,4-oxadiazole-5-carboxamide

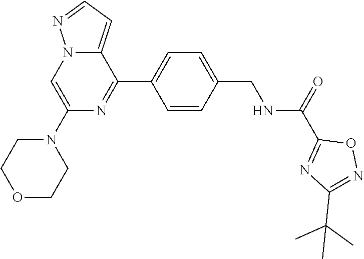 [SMILES: CC(C)(C)c1noc(C(=O)NCc2ccc(-c3nc(N4CCOCC4)cn4nccc34)cc2)n1]

3-(tert-Butyl)-N-(4-(6-morpholinopyrazolo[1,5-a]pyrazin-4-yl)benzyl)-1,2,4-oxadiazole-5-carboxamide was prepared from 5-tert-butyl-1,2,4-oxadiazole-2-carboxylate and (4-(6-morpholinopyrazolo[1,5-a]pyrazin-4-yl)phenyl)methanamine hydrochloride (Example 23, step 3), following the method used in Example 23, step 4. LCMS m/z=462.2 [M+H]⁺. ¹H NMR (DMSO-d₆) δ: 10.04-9.92 (m, 1H), 8.16 (s, 1H), 8.11-8.04 (m, 2H), 8.03 (d, J=2.4 Hz, 1H), 7.53 (d, J=8.55 Hz, 2H), 7.12-7.02 (m, 1H), 4.65-4.45 (m, 2H), 3.87-3.70 (m, 4H), 1.44 (s, 9H).

### Example 25. 2-(tert-butyl)-N-(4-(6-morpholinopyrazolo[1,5-a]pyrazin-4-yl)benzyl)-2H-tetrazole-5-carboxamide

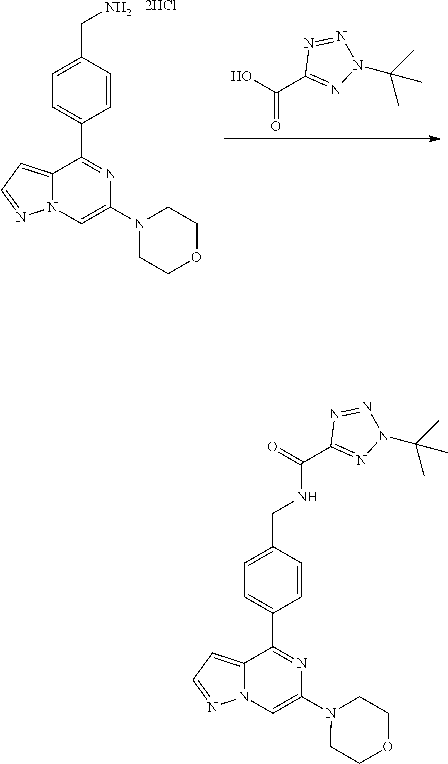 [SMILES: "2HCl" ; NCc1ccc(-c2nc(N3CCOCC3)cn3nccc23)cc1 ; CC(C)(C)n1nnc(C(=O)O)n1 ; CC(C)(C)n1nnc(C(=O)NCc2ccc(-c3nc(N4CCOCC4)cn4nccc34)cc2)n1]

SUBSTRATE STOCK SOLUTION—To a vial containing (4-(6-morpholinopyrazolo[1,5-a]pyrazin-4-yl)phenyl)methanamine dihydrochloride (Example 23, step 3, 105 mg, 0.339 mmol) was added DMF (5 mL) followed by DIPEA (130 mg) and the mixture stirred.

T3P® (50% purity) was added to a mixture of 2-(tert-butyl)-2H-tetrazole-5-carboxylic acid (80 μmol) and substrate stock solution (1 mL) and the reaction stirred at 50° C. for 3 h. The reaction was allowed to cool to RT, diluted with water (6 mL) and left to stand overnight. The vials were centrifuged, mother liquor decanted and discarded. The solids were slurried in water (5 mL), vortexed, centrifuged, mother liquor decanted and discarded. The crude material was dissolved in DMSO (2.2 mL) and purified by prep-HPLC (Method C2, gradient 5-60%) to afford 2-(tert-butyl)-N-(4-(6-morpholinopyrazolo[1,5-a]pyrazin-4-yl)benzyl)-2H-tetrazole-5-carboxamide. LCMS m/z=462.2 [M+H]⁺. ¹H NMR (500 MHz, DMSO-d₆) δ ppm 9.66 (t, J=6.41 Hz, 1H) 8.15 (d, J=1.22 Hz, 1H) 8.06 (d, J=7.94 Hz, 1H) 8.03 (d, J=2.44 Hz, 1H) 7.54 (d, J=8.55 Hz, 1H) 7.10-7.00 (m, 1H) 4.59 (d, J=6.10 Hz, 2H), 3.86-3.72 (m, 4H) 1.74 (s, 9H).

### Example 26 to 29

The compounds in the following table were prepared from (4-(6-morpholinopyrazolo[1,5-a]pyrazin-4-yl)phenyl)methanamine dihydrochloride (Example 23, step 3) and the appropriate carboxylic acid, following the method described in Example 25.

| Example | |
|---|---|
| No | Name/Structure/Carboxylic Acid/Data |
| 26 | 1-(tert-butyl)-N-(4-(6-morpholinopyrazolo[1,5-a]pyrazin-4-yl)benzyl)-1H- 1,2,3-triazole-4-carboxamide 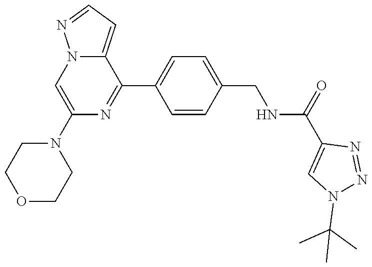 [SMILES: CC(C)(C)n1cc(C(=O)NCc2ccc(-c3nc(N4CCOCC4)cn4nccc34)cc2)nn1] Carboxylic Acid: 1-tert-butyltriazole-4-carboxylic acid Prep-HPLC (Method C2, 5-60% gradient) LCMS m/z = 461.3 [M + H]⁺. ¹H NMR (500 MHz, DMSO-d₆) δ ppm 9.17 (t, J = 6.10 Hz, 1 H) 8.70 (s, 1 H) 8.14 (s, 1 H) 8.10-7.97 (m, 2 H) 7.52 (d, J = 8.55 Hz, 2 H) 7.11-7.01 (m, 1 H) 4.56 (d, J = 6.10 Hz, 2 H) 3.84-3.73 (m, 4 H) 1.64 (s, 9 H). |
| 27 | 1-(tert-butyl)-N-(4-(6-morpholinopyrazolo[1,5-a]pyrazin-4-yl)benzyl)-1H- pyrazole-4-carboxamide 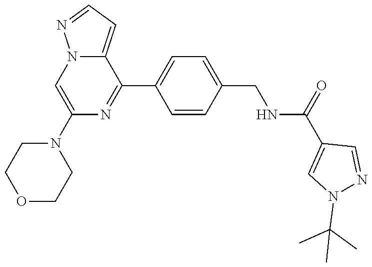 [SMILES: CC(C)(C)n1cc(C(=O)NCc2ccc(-c3nc(N4CCOCC4)cn4nccc34)cc2)cn1] Carboxylic acid: 1-tert-butylpyrazole-4-carboxylic acid Prep HPLC (Method C2, 5-60% gradient) LCMS m/z = 460.2 [M + H]⁺. ¹H NMR (500 MHz, DMSO-d₆) δ ppm 8.66 (t, J = 5.80 Hz, 1 H) 8.32 (s, 1 H) 8.15 (d, J = 1.22 Hz, 1 H) 8.08-7.99 (m, 2 H) 7.92 (s, 1 H) 7.50 (d, J = 8.55 Hz, 2 H) 7.05 (d, J = 1.83 Hz, 1 H) 4.52 (d, J = 6.10 Hz, 2 H) 3.85-3.73 (m, 4 H) 1.53 (s, 9 H) |
| 28 | 1-(tert-butyl)-N-(4-(6-morpholinopyrazolo[1,5-a]pyrazin-4-yl)benzyl)-1H- pyrazole-3-carboxamide 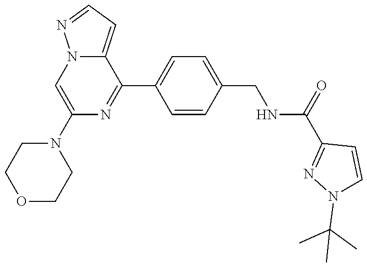 [SMILES: CC(C)(C)n1ccc(C(=O)NCc2ccc(-c3nc(N4CCOCC4)cn4nccc34)cc2)n1] Carboxylic acid: 1-tert-butylpyrazole-3-carboxylic acid Prep-HPLC (Method C2, 5-60% gradient). LCMS m/z = 460.2 [M + H]⁺. ¹H NMR (500 MHz, DMSO-d₆) δ ppm 8.65 (t, J = 6.10 Hz, 1 H) 8.14 (s, 1 H) 8.09-7.97 (m, 2 H) 7.92 (d, J = 2.44 Hz, 1 H) 7.52 (d, J = 7.94 Hz, 2 H) 7.05 (d, J = 2.44 Hz, 1 H) 6.66 (d, J = 2.44 Hz, 1 H) 4.53 (d, J = 6.10 Hz, 2 H) 3.85-3.72 (m, 4 H) 1.73-1.46 (m, 9 H). |
| 29 | 5-(tert-butyl)-N-(4-(6-morpholinopyrazolo[1,5-a]pyrazin-4-yl)benzyl)-1,3,4- oxadiazole-2-carboxamide 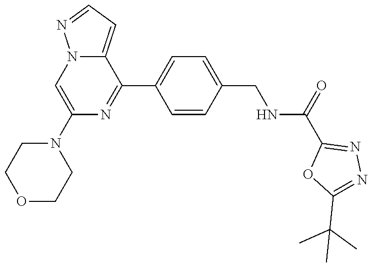 [SMILES: CC(C)(C)c1nnc(C(=O)NCc2ccc(-c3nc(N4CCOCC4)cn4nccc34)cc2)o1] Carboxylic acid: potassium 5-tert-butyl-1,3,4-oxadiazole-2-carboxylate Prep-HPLC (Method C2, 5-60% gradient). LCMS m/z = 462.2 [M + H]⁺ |

### Example 30. 4-isobutyl-1-(2-methyl-4-(6-(1-methy-1H-pyrazol-4-pyrazolo[1,5-a]pyrazin-4-yl)benzyl)piperazin-2-one

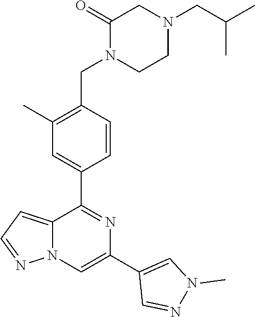 [SMILES: Cc1cc(-c2nc(-c3cnn(C)c3)cn3nccc23)ccc1CN1CCN(CC(C)C)CC1=O]

### 1. Preparation of tert-butyl 4-(4-bromo-2-methylbenzyl)-3-oxopiperazine-1-carboxylate

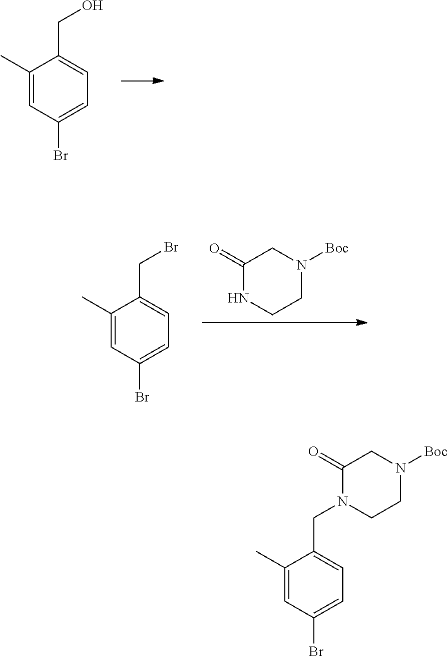

PBr₃ (0.95 mL, 10 mmol) was added dropwise to a solution of (4-bromo-2-methylphenyl)methanol (1.3 g, 6.7 mmol) in DCM (50 mL) at 0° C., the reaction was stirred at RT for 1 h, then quenched with ice-water (50 mL). The pH was adjusted to 7.0 with 50% aq. NaOH solution and the mixture was extracted with EtOAc (100 mL×2). The combined organic layers were washed with water (50 mL), dried (Na₂SO₄) and concentrated to give 4-bromo-1-(bromomethyl)-2-methylbenzene (1.56 g, yield: 89%) as a white solid which was used in the next step without further purification.

To a solution of tert-butyl 3-oxopiperazine-1-carboxylate (1.2 g, 6.0 mmol) in THF (200 mL) was added NaH (320 mg, 8.1 mmol) in an ice-bath and the mixture stirred at 0° C. for 1 h. A solution of 4-bromo-1-(bromomethyl)-2-methylbenzene (1.42 g, 5.4 mmol) in THF (5 mL) was added dropwise over a period of 10 min, the ice-bath was removed and the mixture stirred at RT for 2 h. The mixture was diluted with ice-water (50 mL) and extracted with EtOAc (100 mL×2). The combined organic phases were washed with brine (50 mL), dried (Na₂SO₄) and concentrated in vacuo. The residue was purified by silica gel column (PE/EtOAc=5:1) to give tert-butyl 4-(4-bromo-2-methylbenzyl)-3-oxopiperazine-1-carboxylate (1.6 g, yield: 77%) as white solid. LCMS m/z=327.0[M+H]⁺.

### 2. Preparation of tert-butyl 4-(2-methyl-4-(4,4,5,5-tetramethyl-1,3,2-dioxaborolan-2-yl)benzyl)-3-oxopiperazine-1-carboxylate

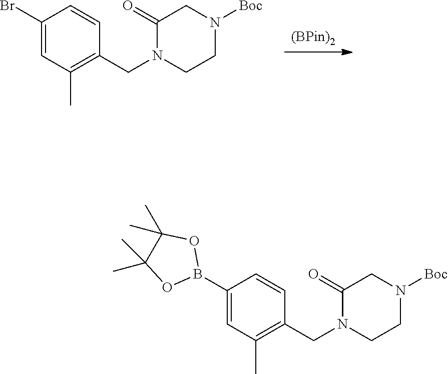 [SMILES: Cc1cc(Br)ccc1CN1CCN(C(=O)OC(C)(C)C)CC1=O ; "(BPin)2" ; Cc1cc(B2OC(C)(C)C(C)(C)O2)ccc1CN1CCN(C(=O)OC(C)(C)C)CC1=O]

A mixture of tert-butyl 4-(4-bromo-2-methylbenzyl)-3-oxopiperazine-1-carboxylate (500 mg, 1.30 mmol), (BPin)₂ (330.12 mg, 1.30 mmol), Pd(dppf)Cl₂ (95.12 mg, 130 μmol) and KOAc (382.75 mg, 3.90 mmol) in dioxane (8.0 mL) was degassed and heated to 95° C. for 16 h. After cooling to RT, the crude was filtered through Celite® and the filtrate concentrated. The residue was chromatographed on silica gel (Heptanes/EtOAc 0-100%) to give tert-butyl 4-(2-methyl-4-(4,4,5,5-tetramethyl-1,3,2-dioxaborolan-2-yl)benzyl)-3-oxopiperazine-1-carboxylate (598 mg, crude). LCMS m/z=375.3 [M+H]⁺.

### 3. Preparation of tert-butyl 4-(2-methyl-4-(6-(1-methyl-1H-pyrazol-4-yl)pyrazolo[1,5-a]pyrazin-4-yl)benzyl)-3-oxopiperazine-1-carboxylate

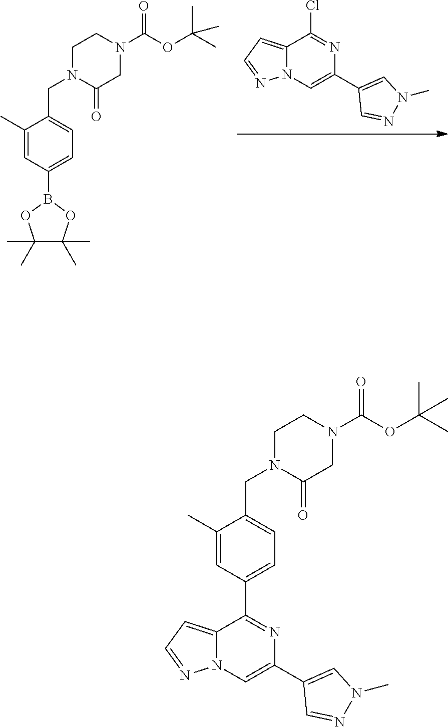

A mixture of tert-butyl 4-(2-methyl-4-(4,4,5,5-tetramethyl-1,3,2-dioxaborolan-2-yl)benzyl)-3-oxopiperazine-1-carboxylate (238 mg, 553.05 μmol), 4-chloro-6-(1-methyl-1H-pyrazol-4-yl)pyrazolo[1,5-a]pyrazine (Example 1, step 5, 129.23 mg, 553.05 μmol), Pd(dppf)Cl₂ (40.47 mg, 55.30 μmol) and K₂CO₃ (229.31 mg, 1.66 mmol) in dioxane (3.0 mL) and water (1.0 mL) was degassed and heated to 95° C. for 16 h. The cooled mixture was filtered through Celite® and concentrated in vacuo. The residue was chromatographed on silica gel (Heptanes/EtOAc 0-100%) to give tert-butyl 4-(2-methyl-4-(6-(1-methyl-1H-pyrazol-4-yl)pyrazolo[1,5-a]pyrazin-4-yl)benzyl)-3-oxopiperazine-1-carboxylate (136 mg, 49% yield). LCMS m/z=502.3 [M+H]⁺.

### 4. Preparation of 1-(2-methyl-4-(6-(1-methyl-1H-pyrazol-4-yl)pyrazolo[1,5-a]pyrazin-4-yl)benzyl)piperazin-2-one hydrochloride

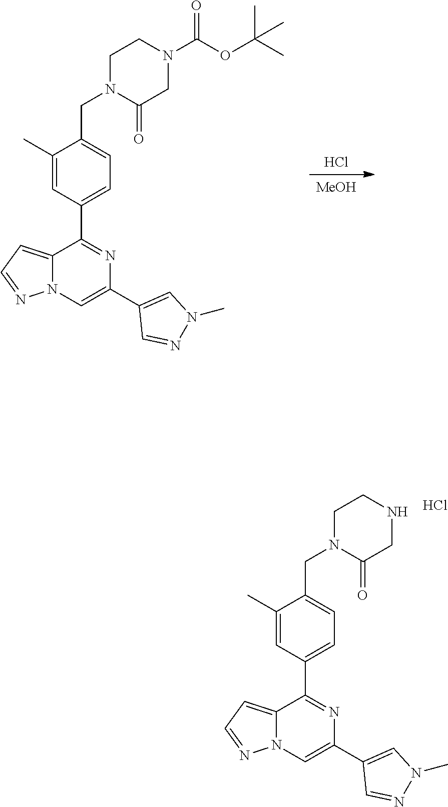 [SMILES: "MeOH" ; Cc1cc(-c2nc(-c3cnn(C)c3)cn3nccc23)ccc1CN1CCN(C(=O)OC(C)(C)C)CC1=O ; Cl ; Cc1cc(-c2nc(-c3cnn(C)c3)cn3nccc23)ccc1CN1CCNCC1=O ; Cl]

HCl (4 M, 203.36 uL) was added to a solution of tert-butyl 4-(2-methyl-4-(6-(1-methyl-1H-pyrazol-4-yl)pyrazolo[1,5-a]pyrazin-4-yl)benzyl)-3-oxopiperazine-1-carboxylate (136 mg, 271.14 μmol) in MeOH (2.0 mL) and the reaction stirred at RT for 16 h. The reaction mixture was evaporated under reduced pressure to give 1-(2-methyl-4-(6-(1-methyl-1H-pyrazol-4-yl)pyrazolo[1,5-a]pyrazin-4-yl)benzyl)piperazin-2-one hydrochloride (114 mg, crude) which was used without further purification. LCMS m/z=402.3 [M+H]⁺

### 5. Preparation of 4-isobutyl-1-(2-methyl-4-(6-(1-methyl-1H-pyrazol-4-yl)pyrazolo[1,5-a]pyrazin-4-yl)benzyl)piperazin-2-one

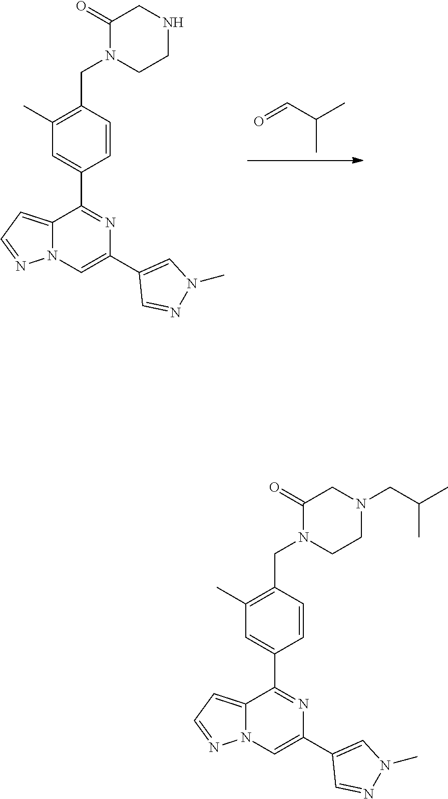

NaBH(OAc)₃ (62.09 mg, 292.94 μmol) was added to a solution of 1-(2-methyl-4-(6-(1-methyl-1H-pyrazol-4-yl)pyrazolo[1,5-a]pyrazin-4-yl)benzyl)piperazin-2-one hydrochloride (58.80 mg, 146.47 μmol), isobutyraldehyde (25.23 mg, 351.53 μmol) and AcOH (8.80 mg, 146.47 μmol) in DCM (2.0 mL) and the reaction stirred at RT for 16 h. The mixture was quenched with MeOH and concentrated in vacuo. The residue was purified by prep HPLC to give 4-isobutyl-1-(2-methyl-4-(6-(1-methyl-1H-pyrazol-4-yl)pyrazolo[1,5-a]pyrazin-4-yl)benzyl)piperazin-2-one (53 mg, 75% yield). LCMS m/z=458.3 [M+H]⁺. ¹H NMR (400 MHz, MeOH-d₄) δ: 8.84-8.75 (m, 1H), 8.24-8.17 (m, 1H), 8.13-8.04 (m, 2H), 8.00-7.88 (m, 2H), 7.51-7.41 (m, 1H), 7.09-7.02 (m, 1H), 4.85 (br s, 2H), 4.13-4.05 (m, 2H), 4.01-3.92 (m, 3H), 3.70-3.57 (m, 4H), 3.18-3.08 (m, 2H), 2.54-2.45 (m, 3H), 2.29-2.14 (m, 1H), 1.08 (d, J=6.78 Hz, 6H).

### Example 31. 1-(2-methyl-4-(6-(1-methyl-1H-pyrazol-4-yl)pyrazolo[1,5-a]pyrazin-4-yl)benzyl)-4-neopentylpiperazin-2-one

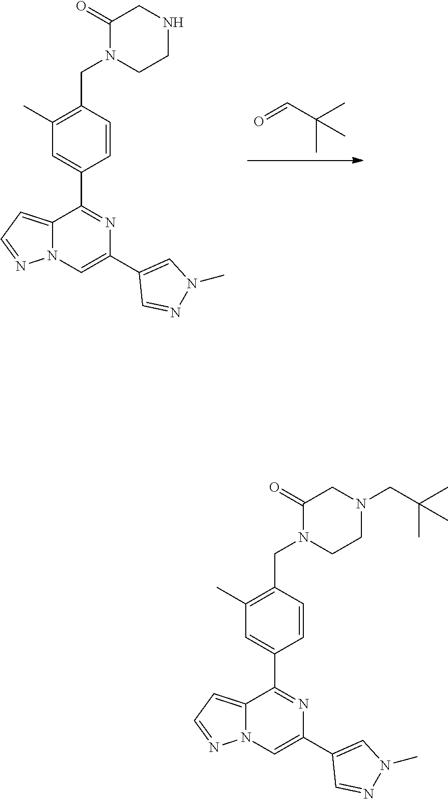

1-(2-Methyl-4-(6-(1-methyl-1H-pyrazol-4-yl)pyrazolo[1,5-a]pyrazin-4-yl)benzyl)-4-neopentylpiperazin-2-one was obtained (25.1 mg, 40% yield) from 1-(2-methyl-4-(6-(1-methyl-1H-pyrazol-4-yl)pyrazolo[1,5-a]pyrazin-4-yl)benzyl)piperazin-2-one hydrochloride (Example 30, step 4) and pivaldehyde following the method described in Example 30, step 5. LCMS m/z=472.2 [M+H]⁺. ¹H NMR (400 MHz, MeOH-d₄) δ: 8.87-8.79 (m, 1H), 8.26-8.19 (m, 1H), 8.12-8.06 (m, 2H), 8.01-7.91 (m, 2H), 7.50-7.41 (m, 1H), 7.10-7.02 (m, 1H), 4.85-4.80 (m, 2H), 4.10-4.03 (m, 2H), 4.00-3.94 (m, 3H), 3.67-3.53 (m, 4H), 3.14-3.04 (m, 2H), 2.53-2.46 (m, 3H), 1.14 (s, 9H).

### Example 32. 1-(2-methyl-4-(6-(1-methyl-1H-pyrazol-4-yl)pyrazolo[1,5-a]pyrazin-4-yl)benzyl)-4-(oxetan-3-ylmethyl)piperazin-2-one

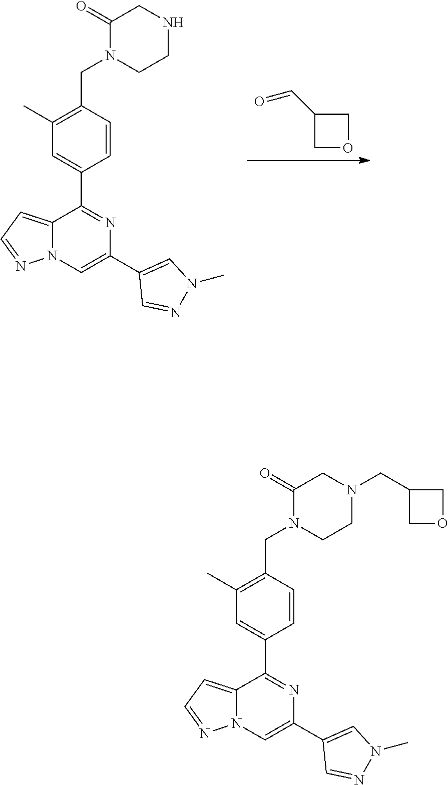 [SMILES: Cc1cc(-c2nc(-c3cnn(C)c3)cn3nccc23)ccc1CN1CCNCC1=O ; O=CC1COC1 ; Cc1cc(-c2nc(-c3cnn(C)c3)cn3nccc23)ccc1CN1CCN(CC2COC2)CC1=O]

1-(2-Methyl-4-(6-(1-methyl-1H-pyrazol-4-yl)pyrazolo[1,5-a]pyrazin-4-yl)benzyl)-4-(oxetan-3-ylmethyl)piperazin-2-one was obtained (19.2 mg, 52% yield) from 1-(2-methyl-4-(6-(1-methyl-1H-pyrazol-4-yl)pyrazolo[1,5-a]pyrazin-4-yl)benzyl)piperazin-2-one hydrochloride (Example 30, step 4) and oxetane-3-carbaldehyde following the method described in Example 30, step 5. LCMS m/z=472.1 [M+H]⁺. ¹H NMR (400 MHz, MeOH-d₄) δ: 8.84-8.78 (m, 1H), 8.23-8.19 (m, 1H), 8.12-8.05 (m, 2H), 8.00-7.91 (m, 2H), 7.48-7.39 (m, 1H), 7.08-7.03 (m, 1H), 4.96-4.91 (m, 1H), 4.83 (br s, 2H), 4.55-4.49 (m, 2H), 4.14-4.09 (m, 1H), 4.00-3.98 (m, 5H), 3.74 (dd, J=4.6, 10.7 Hz, 1H), 3.64-3.44 (m, 6H), 2.47 (br s, 3H).

### Example 33. 1-(4-(6-(1-methyl-1H-pyrazol-4-yl)pyrazolo[1,5-a]pyrazin-4-yl)benzyl)-4-neopentylpiperazin-2-one

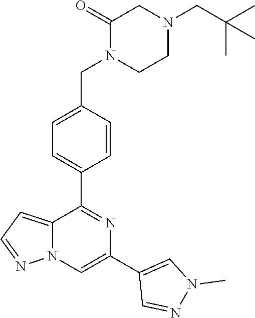 [SMILES: Cn1cc(-c2cn3nccc3c(-c3ccc(CN4CCN(CC(C)(C)C)CC4=O)cc3)n2)cn1]

### 1. Preparation of tert-butyl 4-(4-bromobenzyl)-3-oxopiperazine-1-carboxylate

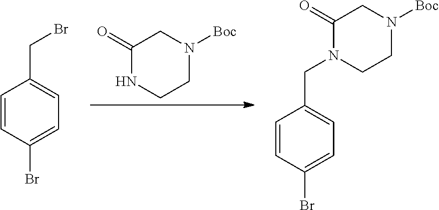

NaH (57.60 mg, 2.40 mmol) was added to a solution of tert-butyl 3-oxopiperazine-1-carboxylate (400 mg, 2.0 mmol) in THF (4.0 mL) at 0° C., the mixture stirred for 1 h, 1-bromo-4-(chloromethyl)benzene (410.96 mg, 2.0 mmol) was added and the reaction stirred at RT for 2 h. The reaction was quenched with aq. NaHCO₃, extracted with EtOAc, the organic layer was dried over Na₂SO₄ and concentrated in vacuum. The residue was chromatographed on silica gel (Heptanes/EtOAc 0-100%) to give tert-butyl 4-(4-bromobenzyl)-3-oxopiperazine-1-carboxylate (54.50 mg, 7.4% yield). 2. Preparation of tert-butyl 4-(4-(4,4,5,5-tetramethyl-1,3,2-dioxaborolan-2-yl)benzyl)-3-oxopiperazine-1-carboxylate

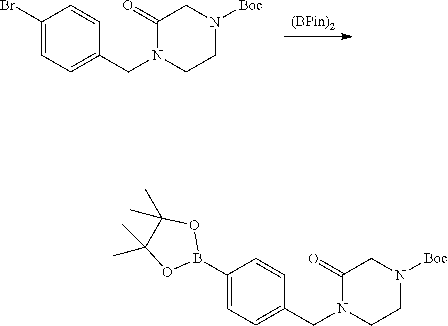 [SMILES: CC(C)(C)OC(=O)N1CCN(Cc2ccc(Br)cc2)C(=O)C1 ; "(BPin)2" ; CC(C)(C)OC(=O)N1CCN(Cc2ccc(B3OC(C)(C)C(C)(C)O3)cc2)C(=O)C1]

A mixture of tert-butyl 4-((4-bromophenyl)methyl)-3-oxo-piperazine-1-carboxylate (54.30 mg, 147.05 μmol), (BPin)₂ (37.34 mg, 147.05 μmol), KOAc (43.29 mg, 441.15 μmol) and Pd(dppf)Cl₂·DCM (12.01 mg, 14.71 μmol) in dioxane (2.0 mL) was degassed and heated at 95° C. for 16 h. The cooled mixture was filtered through Celite® and the filtrate concentrated. The residue was chromatographed on silica gel (Heptanes/EtOAc 0-100%) to give tert-butyl 4-(4-(4,4,5,5-tetramethyl-1,3,2-dioxaborolan-2-yl)benzyl)-3-oxopiperazine-1-carboxylate (53.30 mg, 87.1% yield).

### 3. Preparation of tert-butyl 4-(4-(6-(1-methyl-1H-pyrazol-4-yl)pyrazolo[1,5-a]pyrazin-4-yl)benzyl)-3-oxopiperazine-1-carboxylate

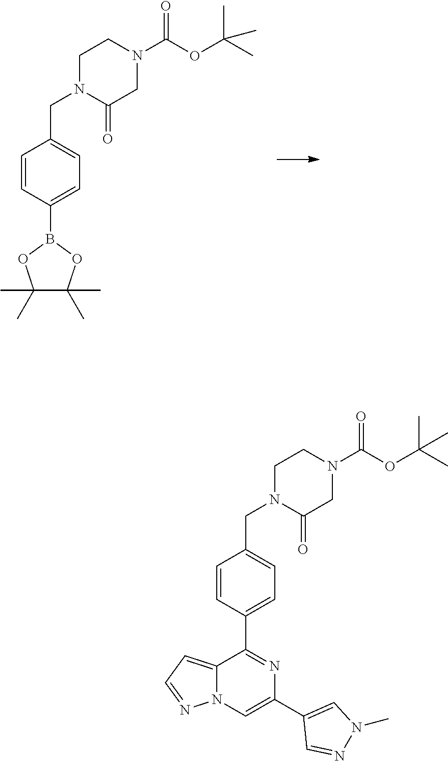 [SMILES: CC(C)(C)OC(=O)N1CCN(Cc2ccc(B3OC(C)(C)C(C)(C)O3)cc2)C(=O)C1 ; Cn1cc(-c2cn3nccc3c(-c3ccc(CN4CCN(C(=O)OC(C)(C)C)CC4=O)cc3)n2)cn1]

tert-Butyl 4-(4-(6-(1-methyl-1H-pyrazol-4-yl)pyrazolo[1,5-a]pyrazin-4-yl)benzyl)-3-oxopiperazine-1-carboxylate was obtained (31.3 mg, 50.1% yield) from tert-butyl 4-(4-(4,4,5,5-tetramethyl-1,3,2-dioxaborolan-2-yl)benzyl)-3-oxopiperazine-1-carboxylate and 4-chloro-6-(1-methyl-1H-pyrazol-4-yl)pyrazolo[1,5-a]pyrazine (Example 1, step 5), following a similar method to that used in Example 30, step 3. LCMS m/z=488.4 [M+H]⁺

### 4. Preparation of 1-(4-(6-(1-methyl-1H-pyrazol-4-yl)pyrazolo[1,5-a]pyrazin-4-yl)benzyl)piperazin-2-one hydrochloride

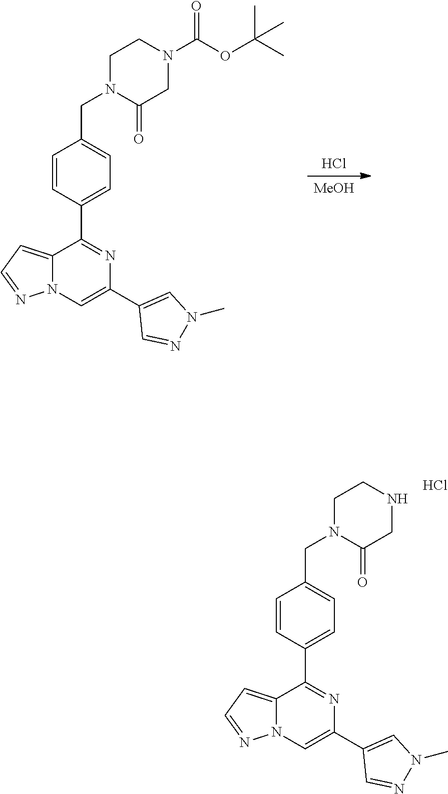 [SMILES: "MeOH" ; Cn1cc(-c2cn3nccc3c(-c3ccc(CN4CCN(C(=O)OC(C)(C)C)CC4=O)cc3)n2)cn1 ; Cl ; Cn1cc(-c2cn3nccc3c(-c3ccc(CN4CCNCC4=O)cc3)n2)cn1 ; Cl]

1-(4-(6-(1-Methyl-1H-pyrazol-4-yl)pyrazolo[1,5-a]pyrazin-4-yl)benzyl)piperazin-2-one hydrochloride was obtained crude, from tert-butyl 4-(4-(6-(1-methyl-1H-pyrazol-4-yl)pyrazolo[1,5-a]pyrazin-4-yl)benzyl)-3-oxopiperazine-1-carboxylate following the method used in Example 30, Step 4. LCMS m/z=388.3 [M+H]⁺.

### 5. Preparation of 1-(4-(6-(1-methyl-1H-pyrazol-4-yl)pyrazolo[1,5-a]pyrazin-4-yl)benzyl)-4-neopentylpiperazin-2-one

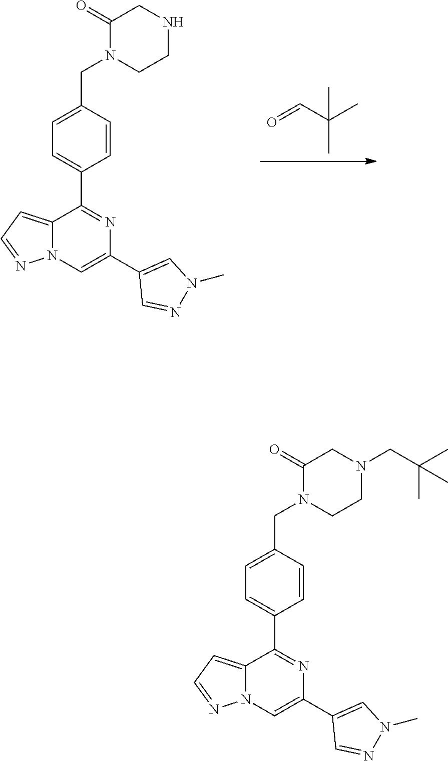

1-(4-(6-(1-Methyl-1H-pyrazol-4-yl)pyrazolo[1,5-a]pyrazin-4-yl)benzyl)-4-neopentyl piperazin-2-one was obtained (14 mg, 95% purity) from 1-(4-(6-(1-methyl-1H-pyrazol-4-yl)pyrazolo[1,5-a]pyrazin-4-yl)benzyl)piperazin-2-one hydrochloride and pivaldehyde, following a similar method to that used for Example 30, step 5. LCMS m/z=458.3 [M+H]⁺. ¹H NMR (400 MHz, CDCl₃) δ: 8.58 (s, 1H), 8.11-8.04 (m, 3H), 8.02-7.93 (m, 2H), 7.50-7.40 (m, 2H), 7.00-6.92 (m, 1H), 4.83-4.73 (m, 2H), 4.07-3.99 (m, 3H), 3.94-3.86 (m, 2H), 3.60-3.53 (m, 2H), 3.39-3.32 (m, 2H), 2.85-2.78 (m, 2H), 1.12 (s, 9H).

### Example 34. 1-(2-fluoro-4-(6-(1-methyl-1H-pyrazol-4-yl)pyrazolo[1,5-a]pyrazin-4-yl)benzyl)-4-isobutylpiperazin-2-one

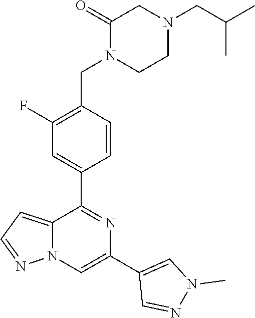 [SMILES: CC(C)CN1CCN(Cc2ccc(-c3nc(-c4cnn(C)c4)cn4nccc34)cc2F)C(=O)C1]

### 1. Preparation of tert-butyl 4-(4-bromo-2-fluorobenzyl)-3-oxopiperazine-1-carboxylate

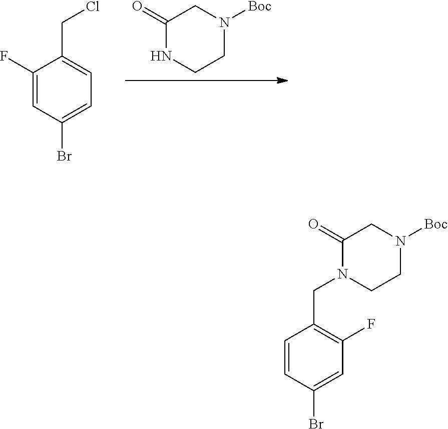

tert-Butyl 4-(4-bromo-2-fluorobenzyl)-3-oxopiperazine-1-carboxylate was obtained (420 mg, 20.7% yield) from 4-bromo-1-(chloromethyl)-2-fluorobenzene and tert-butyl 3-oxopiperazine-1-carboxylate, following a similar method to that used in Example 33, step 1. 1H NMR (400 MHz, CDCl₃) δ: 7.31-7.27 (m, 2H), 7.26-7.22 (m, 1H), 4.63 (s, 2H), 4.14-4.12 (m, 2H), 3.65-3.60 (m, 2H), 3.36-3.31 (m, 2H), 1.47 (s, 9H).

### 2. Preparation of tert-butyl 4-(2-fluoro-4-(4,4,5,5-tetramethyl-1,3,2-dioxaborolan-2-yl)benzyl)-3-oxopiperazine-1-carboxylate

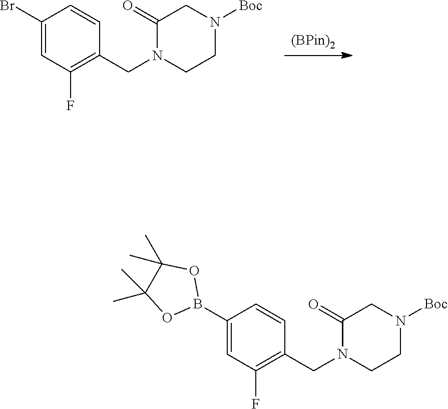 [SMILES: CC(C)(C)OC(=O)N1CCN(Cc2ccc(Br)cc2F)C(=O)C1 ; "(BPin)2" ; CC(C)(C)OC(=O)N1CCN(Cc2ccc(B3OC(C)(C)C(C)(C)O3)cc2F)C(=O)C1]

tert-Butyl 4-(2-fluoro-4-(4,4,5,5-tetramethyl-1,3,2-dioxaborolan-2-yl)benzyl)-3-oxopiperazine-1-carboxylate was obtained (235 mg crude) from tert-butyl 4-(4-bromo-2-fluorobenzyl)-3-oxopiperazine-1-carboxylate following a similar method to that used in Example 33, step 2. 3. Preparation of tert-butyl 4-(2-fluoro-4-(6-(1-methyl-1H-pyrazol-4-yl)pyrazolo[1,5-a]pyrazin-4-yl)benzyl)-3-oxopiperazine-1-carboxylate

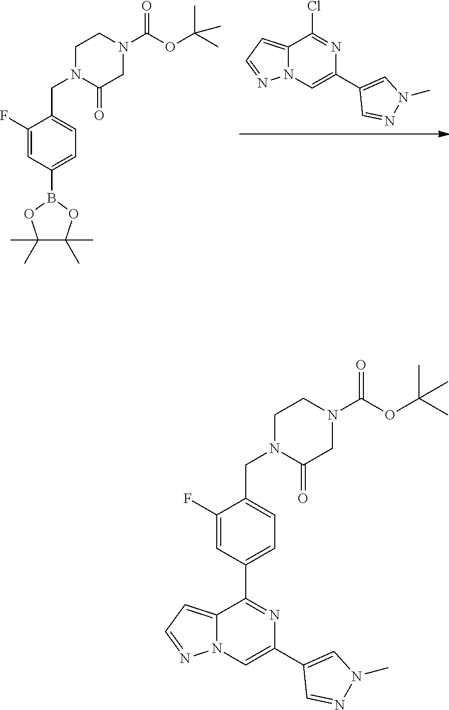

tert-Butyl 4-(2-fluoro-4-(6-(1-methyl-1H-pyrazol-4-yl)pyrazolo[1,5-a]pyrazin-4-yl)benzyl)-3-oxopiperazine-1-carboxylate was obtained (54 mg, 19.7% yield) from tert-butyl 4-(2-fluoro-4-(4,4,5,5-tetramethyl-1,3,2-dioxaborolan-2-yl)benzyl)-3-oxopiperazine-1-carboxylate and 4-chloro-6-(1-methyl-1H-pyrazol-4-yl)pyrazolo[1,5-a]pyrazine (Example 1, step 5), following a similar method to that used in Example 30, step 3. LCMS m/z=506.3 [M+H]⁺.

### 4. Preparation of 1-(2-fluoro-4-(6-(1-methyl-1H-pyrazol-4-yl)pyrazolo[1,5-a]pyrazin-4-yl)benzyl)piperazin-2-one hydrochloride

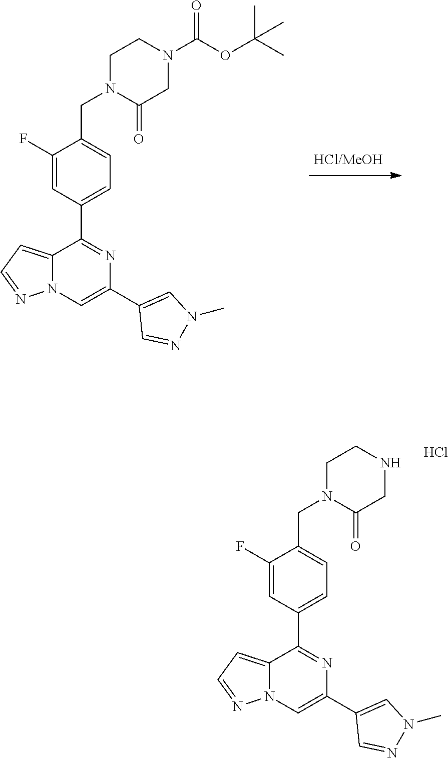 [SMILES: Cn1cc(-c2cn3nccc3c(-c3ccc(CN4CCN(C(=O)OC(C)(C)C)CC4=O)c(F)c3)n2)cn1 ; Cn1cc(-c2cn3nccc3c(-c3ccc(CN4CCNCC4=O)c(F)c3)n2)cn1 ; Cl]

1-(2-Fluoro-4-(6-(1-methyl-1H-pyrazol-4-yl)pyrazolo[1,5-a]pyrazin-4-yl)benzyl) piperazin-2-one hydrochloride was obtained crude, from tert-butyl 4-(2-fluoro-4-(6-(1-methyl-1H-pyrazol-4-yl)pyrazolo[1,5-a]pyrazin-4-yl)benzyl)-3-oxopiperazine-1-carboxylate following the method used in Example 30, Step 4. LCMS m/z=406.3 [M+H]⁺

### 5. Preparation of 1-(2-fluoro-4-(6-(1-methyl-1H-pyrazol-4-yl)pyrazolo[1,5-a]pyrazin-4-yl)benzyl)-4-isobutylpiperazin-2-one

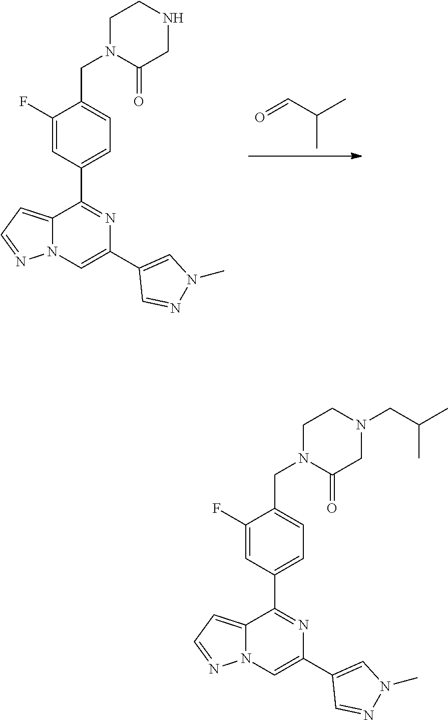

1-(2-Fluoro-4-(6-(1-methyl-1H-pyrazol-4-yl)pyrazolo[1,5-a]pyrazin-4-yl)benzyl)-4-isobutylpiperazin-2-one was obtained (4 mg, 95% purity) from 1-(2-fluoro-4-(6-(1-methyl-1H-pyrazol-4-yl)pyrazolo[1,5-a]pyrazin-4-yl)benzyl)piperazin-2-one hydrochloride and isobutyraldehyde following a similar method to that used for Example 30, step 5. LCMS m/z=462.3 [M+H]^{+ 1}H NMR (400 MHz, CDCl₃, d): 8.61-8.53 (m, 1H), 8.09-8.03 (m, 1H), 8.00-7.94 (m, 2H), 7.90-7.86 (m, 1H), 7.85-7.79 (m, 1H), 7.58-7.51 (m, 1H), 6.99-6.94 (m, 1H), 4.80-4.70 (m, 2H), 4.00 (s, 3H), 3.41-3.33 (m, 2H), 3.24-3.18 (m, 2H), 2.70-2.62 (m, 2H), 2.20-2.13 (m, 2H), 1.87-1.73 (m, 1H), 0.92 (d, J=6.5 Hz, 6H).

### Example 35. 1-(2-fluoro-4-(6-(1-methyl-1H-pyrazol-4-yl)pyrazolo[1,5-a]pyrazin-4-yl)benzyl)-4-neopentylpiperazin-2-one

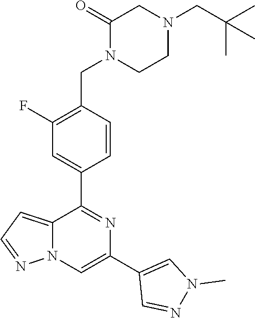 [SMILES: Cn1cc(-c2cn3nccc3c(-c3ccc(CN4CCN(CC(C)(C)C)CC4=O)c(F)c3)n2)cn1]

1-(2-Fluoro-4-(6-(1-methyl-1H-pyrazol-4-yl)pyrazolo[1,5-a]pyrazin-4-yl)benzyl)-4-neopentylpiperazin-2-one was obtained (15 mg) from 1-(2-fluoro-4-(6-(1-methyl-1H-pyrazol-4-yl)pyrazolo[1,5-a]pyrazin-4-yl)benzyl)piperazin-2-one hydrochloride (Example 34, step 4) and pivaldehyde, following a similar method to that used for Example 30, step 5. LCMS m/z=476.4 [M+H]⁺. ¹H NMR (400 MHz, CDCl₃, d): 8.62-8.57 (m, 1H), 8.11-8.05 (m, 1H), 8.03-7.96 (m, 2H), 7.93-7.79 (m, 2H), 7.63-7.49 (m, 1H), 7.04-6.92 (m, 1H), 4.88-4.75 (m, 2H), 4.03 (s, 3H), 3.93-3.86 (m, 2H), 3.72-3.64 (m, 2H), 3.45-3.36 (m, 2H), 2.88-2.79 (m, 2H), 1.12 (s, 9H).

### Example 36: 4-isobutyl-3-methyl-1-(2-methyl-4-(6-(1-methyl-1H-pyrazol-4-yl)pyrazolo[1,5-a]pyrazin-4-yl)benzyl)piperazin-2-one

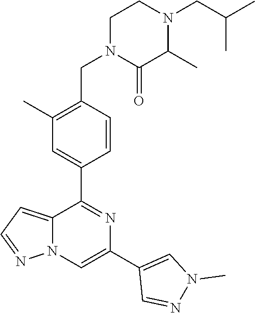 [SMILES: Cc1cc(-c2nc(-c3cnn(C)c3)cn3nccc23)ccc1CN1CCN(CC(C)C)C(C)C1=O]

### 1. Preparation of tert-butyl 4-((4-bromo-2-methyl-phenyl)methyl)-2-methyl-3-oxo-piperazine-1-carboxylate

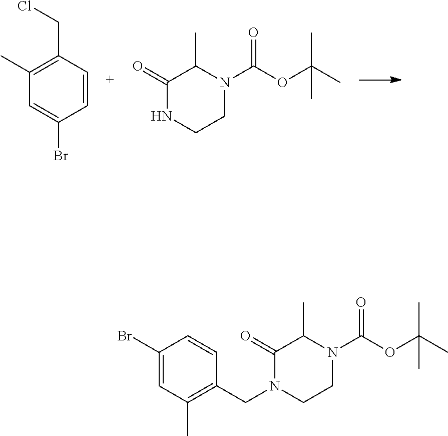

A mixture of 4-bromo-1-(chloromethyl)-2-methyl-benzene (1.02 g, 4.67 mmol), tert-butyl 2-methyl-3-oxo-piperazine-1-carboxylate (1.00 g, 4.67 mmol) and sodium tert-butoxide (1.35 g, 14.0 mmol) in dioxane (5.0 mL) was heated at 95° C. overnight. The cooled mixture was diluted with EtOAc, washed with water and dried over Na₂SO₄. The concentrated residue was chromatographed on silica gel (Heptanes/EtOAc 0-100%) to give tert-butyl 4-((4-bromo-2-methyl-phenyl)methyl)-2-methyl-3-oxo-piperazine-1-carboxylate (1.37 g, 70% yield). LCMS m/z=297.0 [M+H]⁺

### 2. Preparation of tert-butyl 2-methyl-4-(2-methyl-4-(4,4,5,5-tetramethyl-1,3,2-dioxaborolan-2-yl)benzyl)-3-oxopiperazine-1-carboxylate

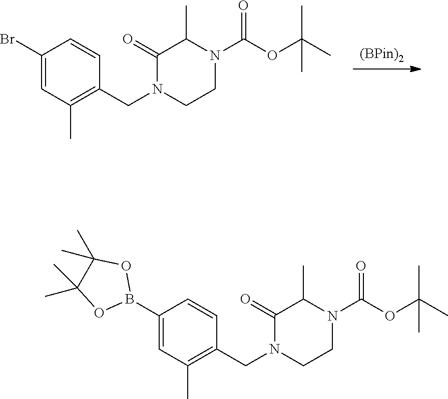

tert-Butyl 2-methyl-4-(2-methyl-4-(4,4,5,5-tetramethyl-1,3,2-dioxaborolan-2-yl)benzyl)-3-oxopiperazine-1-carboxylate was obtained (1.41 g, 92% yield) from tert-butyl 4-((4-bromo-2-methyl-phenyl)methyl)-2-methyl-3-oxo-piperazine-1-carboxylate, following a similar method to that used in Example 30, step 2.

### 3. Preparation of tert-butyl 2-methyl-4-(2-methyl-4-(6-(1-methyl-1H-pyrazol-4-yl)pyrazolo[1,5-a]pyrazin-4-yl)benzyl)-3-oxopiperazine-1-carboxylate

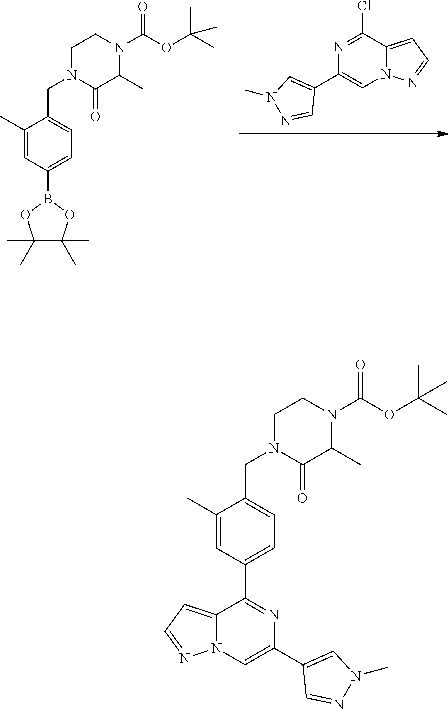 [SMILES: Cc1cc(B2OC(C)(C)C(C)(C)O2)ccc1CN1CCN(C(=O)OC(C)(C)C)C(C)C1=O ; Cn1cc(-c2cn3nccc3c(Cl)n2)cn1 ; Cc1cc(-c2nc(-c3cnn(C)c3)cn3nccc23)ccc1CN1CCN(C(=O)OC(C)(C)C)C(C)C1=O]

tert-Butyl 2-methyl-4-(2-methyl-4-(6-(1-methyl-1H-pyrazol-4-yl)pyrazolo[1,5-a]pyrazin-4-yl)benzyl)-3-oxopiperazine-1-carboxylate was obtained (198.3 mg, 43% yield) from tert-butyl 4-((4-bromo-2-methyl-phenyl)methyl)-2-methyl-3-oxo-piperazine-1-carboxylate and 4-chloro-6-(1-methylpyrazol-4-yl)pyrazolo[1,5-a]pyrazine (Example 1, step 5), following a similar method to that described in Example 30 step 3. LCMS m/z=516.3 [M+H]⁺

### 4. Preparation of 3-methyl-1-(2-methyl-4-(6-(1-methyl-1H-pyrazol-4-yl)pyrazolo[1,5-a]pyrazin-4-yl)benzyl)piperazin-2-one hydrochloride

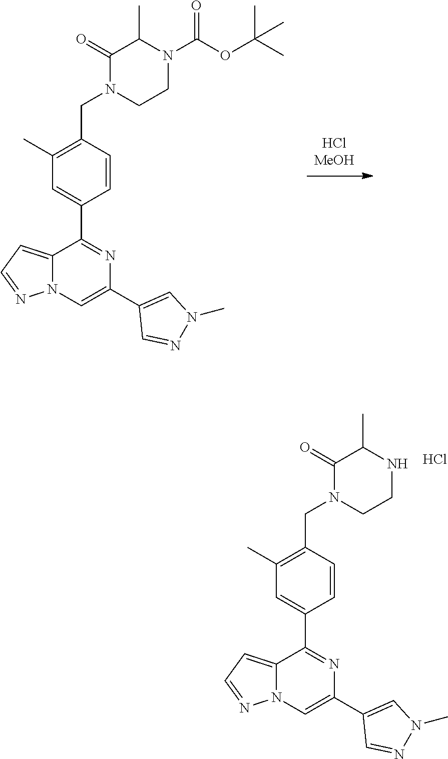 [SMILES: Cc1cc(-c2nc(-c3cnn(C)c3)cn3nccc23)ccc1CN1CCN(C(=O)OC(C)(C)C)C(C)C1=O ; Cc1cc(-c2nc(-c3cnn(C)c3)cn3nccc23)ccc1CN1CCNC(C)C1=O ; Cl]

3-Methyl-1-(2-methyl-4-(6-(1-methyl-1H-pyrazol-4-yl)pyrazolo[1,5-a]pyrazin-4-yl)benzyl)piperazin-2-one hydrochloride was obtained, crude, from tert-butyl 2-methyl-4-(2-methyl-4-(6-(1-methyl-1H-pyrazol-4-yl)pyrazolo[1,5-a]pyrazin-4-yl)benzyl)-3-oxopiperazine-1-carboxylate, following the procedure described in Example 30, step 4. LCMS m/z=416.2 [M+H]⁺

### 5. Preparation of 4-isobutyl-3-methyl-1-(2-methyl-4-(6-(1-methyl-1H-pyrazol-4-yl)pyrazolo[1,5-a]pyrazin-4-yl)benzyl)piperazin-2-one

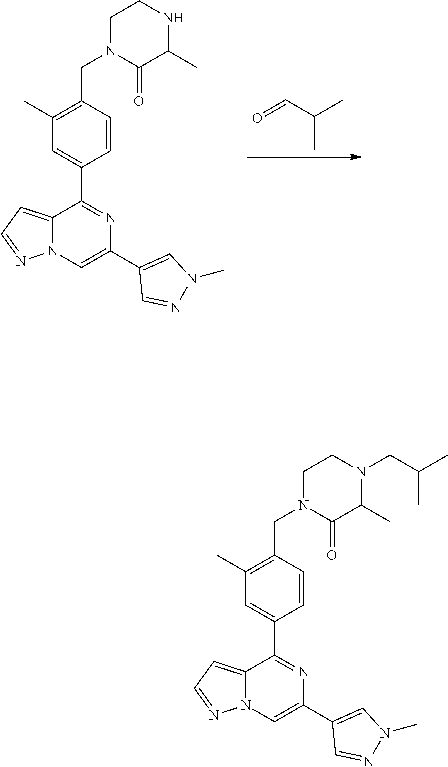

4-Isobutyl-3-methyl-1-(2-methyl-4-(6-(1-methyl-1H-pyrazol-4-yl)pyrazolo[1,5-a]pyrazin-4-yl)benzyl)piperazin-2-one was obtained (39.5 mg, 30% yield) from 3-methyl-1-(2-methyl-4-(6-(1-methyl-1H-pyrazol-4-yl)pyrazolo[1,5-a]pyrazin-4-yl)benzyl)piperazin-2-one hydrochloride and isobutyraldehyde following a similar method to that used in Example 30, step 5. LCMS m/z=472.3 [M+H]⁺. ¹H NMR (400 MHz, CDCl₃) δ: 8.60 (s, 1H), 8.09-8.05 (m, 1H), 8.04 (s, 1H), 8.00 (s, 1H), 7.93-7.86 (m, 2H), 7.32 (d, J=7.8 Hz, 1H), 6.99-6.95 (m, 1H), 4.89 (d, J=15.1 Hz, 1H), 4.70 (d, J=15.1 Hz, 1H), 4.08 (br s, 1H), 4.03 (s, 3H), 3.72-3.39 (m, 4H), 3.11 (dd, J=8.4, 12.9 Hz, 1H), 2.94 (dd, J=5.5, 13.1 Hz, 1H), 2.43 (s, 3H), 2.23-2.10 (m, 1H), 1.79 (d, J=7.3 Hz, 3H), 1.12 (d, J=6.5 Hz, 3H), 1.10-1.07 (m, 3H).

### Example 37: 3-methyl-1-(2-methyl-4-(6-(1-methyl-1H-pyrazol-4-yl)pyrazolo[1,5-a]pyrazin-4-yl)benzyl)-4-(oxetan-3-yl)piperazin-2-one

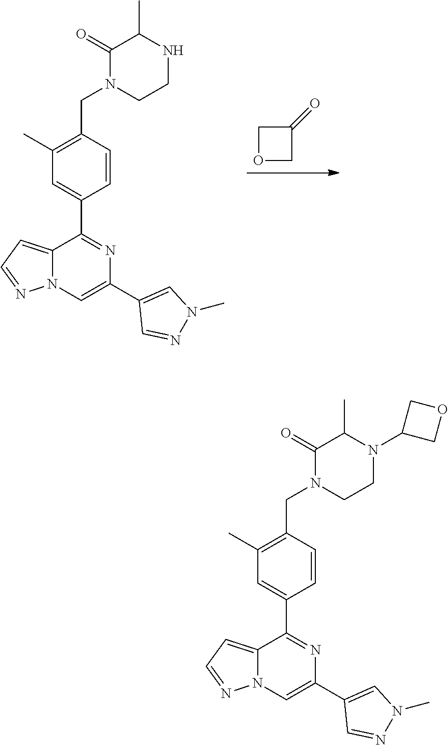 [SMILES: Cc1cc(-c2nc(-c3cnn(C)c3)cn3nccc23)ccc1CN1CCNC(C)C1=O ; O=C1COC1 ; Cc1cc(-c2nc(-c3cnn(C)c3)cn3nccc23)ccc1CN1CCN(C2COC2)C(C)C1=O]

3-Methyl-1-(2-methyl-4-(6-(1-methyl-1H-pyrazol-4-yl)pyrazolo[1,5-a]pyrazin-4-yl)benzyl)-4-(oxetan-3-yl)piperazin-2-one was obtained (67.4 mg, 46% yield) from 3-methyl-1-(2-methyl-4-(6-(1-methyl-1H-pyrazol-4-yl)pyrazolo[1,5-a]pyrazin-4-yl)benzyl)piperazin-2-one hydrochloride (Example 36, step 5) and oxetan-3-one, following a similar method to that used for Example 30, step 5. LCMS m/z=472.2 [M+H]⁺. ¹H NMR (400 MHz, CDCl₃) δ: 8.64 (d, J=1.0 Hz, 1H), 8.10-8.08 (m, 1H), 8.08-8.07 (m, 1H), 8.03-8.01 (m, 1H), 7.92-7.87 (m, 2H), 7.31 (d, J=7.8 Hz, 1H), 7.00 (dd, J=1.0, 2.5 Hz, 1H), 4.93-4.68 (m, 6H), 4.31-4.22 (m, 1H), 4.05 (s, 3H), 3.77 (q, J=7.2 Hz, 1H), 3.50-3.43 (m, 2H), 3.40-3.30 (m, 1H), 3.16-3.04 (m, 1H), 2.43 (s, 3H), 1.53 (d, J=7.0 Hz, 3H).

### Example 38. 2-(tert-butyl)-5-(2-methyl-4-(7-(1-methyl-1H-pyrazol-4-yl)imidazo[1,2-c]pyrimidin-5-yl)benzyl)-4,5-dihydro-6H-pyrrolo[3,4-d]thiazol-6-one

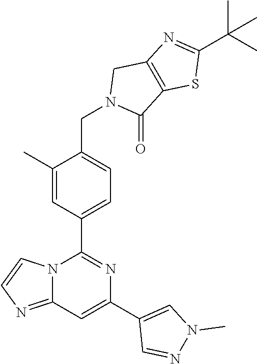 [SMILES: Cc1cc(-c2nc(-c3cnn(C)c3)cc3nccn23)ccc1CN1Cc2nc(C(C)(C)C)sc2C1=O]

### 1. Preparation of 4-(((4-bromo-2-methylbenzyl)amino)methyl)-2-(tert-butyl)thiazole-5-carboxylic acid

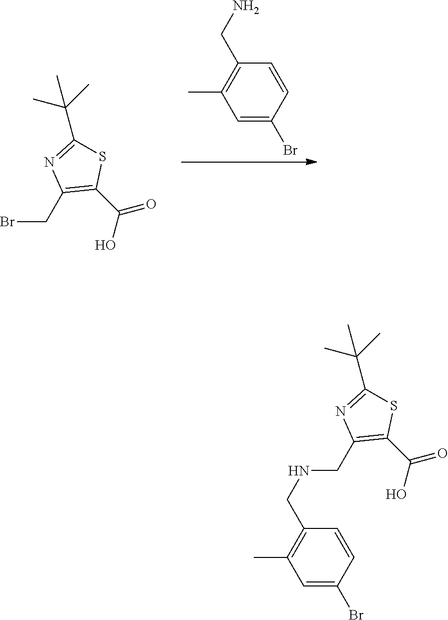

To a solution of 4-bromo-2-methylbenzyl amine hydrochloride (1.68 g, 6.82 mmol) in DMF (15 mL) were added DIPEA (3.0 mL, 22.8 mmol) and 4-(bromomethyl)-2-(tert-butyl)thiazole-5-carboxylate (1.39 g, 4.54 mmol) in DMF (10 mL) at 0° C. The reaction was allowed to warm to RT and stirred for 12 h. The mixture was diluted with water (40 mL) and extracted with EtOAc (100 mL×2). The organic phase was washed with water (60 mL×2), dried and concentrated. The residue was purified by silica gel column chromatography (EtOAc/PE=1/4) to give 4-(((4-bromo-2-methylbenzyl)amino)methyl)-2-(tert-butyl)thiazole-5-carboxylic acid as a yellow oil (850 mg, 44%). LCMS m/z=425.1 [M+H]⁺

### 2. Preparation of 5-((4-bromo-2-methyl-phenyl)methyl)-2-tert-butyl-4H-pyrrolo[3,4-d]thiazol-6-one

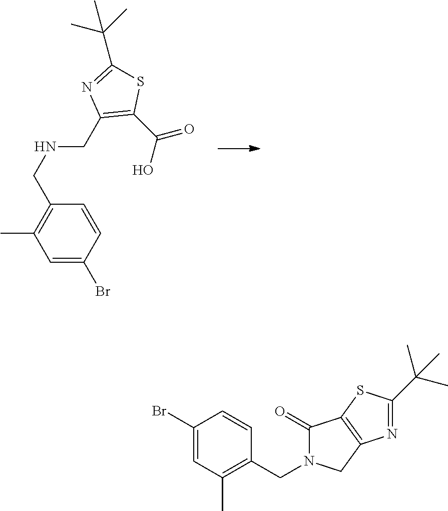 [SMILES: Cc1cc(Br)ccc1CNCc1nc(C(C)(C)C)sc1C(=O)O ; Cc1cc(Br)ccc1CN1Cc2nc(C(C)(C)C)sc2C1=O]

To a solution of 4-(((4-bromo-2-methylbenzyl)amino)methyl)-2-(tert-butyl)thiazole-5-carboxylic acid (850 mg, 2.0 mmol) in MeOH (10 mL) and H₂O (5 mL) was added NaOH (320 mg, 8.0 mmol) and the reaction mixture was stirred at RT for 16 h. After removal of MeOH, the residue was diluted with water (10 mL) and the pH adjusted to 5-6 with HCl (1 N). The mixture was extracted with EtOAc (50 mL×3). The organic layer was dried over Na₂SO₄ and concentrated under reduced pressure to give 5-((4-bromo-2-methyl-phenyl)methyl)-2-tert-butyl-4H-pyrrolo[3,4-d]thiazol-6-one as a yellow solid (760 mg, yield: 96%), which was used in the next step without further purification. LCMS m/z=379.1 [M+H]⁺.

### 3. Preparation of 2-tert-butyl-5-((2-methyl-4-(4,4,5,5-tetramethyl-1,3,2-dioxaborolan-2-yl)phenyl)methyl])-4H-pyrrolo[3,4-d]thiazol-6-one

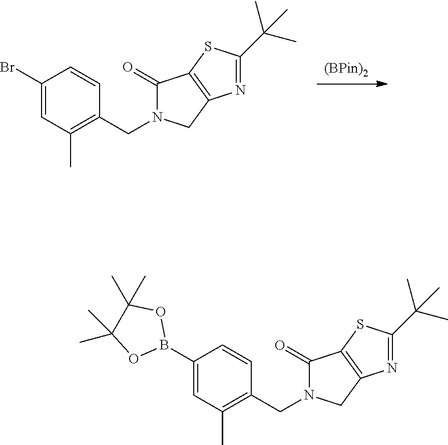

2-tert-Butyl-5-((2-methyl-4-(4,4,5,5-tetramethyl-1,3,2-dioxaborolan-2-yl)phenyl) methyl])-4H-pyrrolo[3,4-d]thiazol-6-one was obtained (24.1 mg, 88% yield) from 5-((4-bromo-2-methyl-phenyl)methyl)-2-tert-butyl-4H-pyrrolo[3,4-d]thiazol-6-one, following a similar method to that used for Example 30, step 2. LCMS m/z=427.3 [M+H]⁺

### 4. Preparation of 2-tert-butyl-5-((2-methyl-4-[7-(1-methylpyrazol-4-yl)imidazo[1,2-c]pyrimidin-5-yl]phenyl)methyl)-4H-pyrrolo[3,4-d]thiazol-6-one

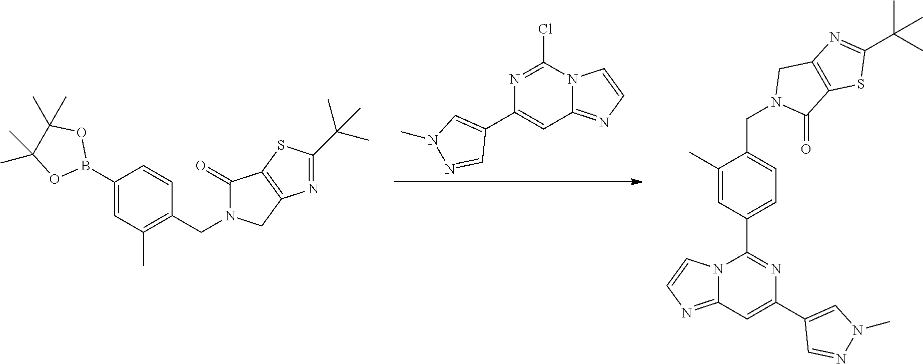

A mixture of 2-tert-butyl-5-((2-methyl-4-(4,4,5,5-tetramethyl-1,3,2-dioxaborolan-2-yl)phenyl)methyl])-4H-pyrrolo[3,4-d]thiazol-6-one (24.0 mg, 56.29 μmol), 5-chloro-7-(1-methylpyrazol-4-yl)imidazo[1,2-c]pyrimidine (Example 1, step 5, 13.15 mg, 56.29 μmol), Pd(dppf)Cl₂ (4.12 mg, 5.63 μmol) and K₂CO₃ (23.34 mg, 168.87 μmol) in dioxane (2.0 mL) and water (199.98 uL) was degassed and heated at 95° C. for 16 h. The cooled mixture was diluted with EtOAc, filtered through Celite®, and the filtrate concentrated in vacuo. The residue was purified by chromatography on silica gel (DCM/MeOH 0-30%) to give 2-tert-butyl-5-((2-methyl-4-[7-(1-methylpyrazol-4-yl)imidazo[1,2-c]pyrimidin-5-yl]phenyl)methyl)-4H-pyrrolo[3,4-d]thiazol-6-one (20.0 mg, 68% yield). LCMS m/z=498.2 [M+H]⁺. ¹H NMR (400 MHz, MeOH-d₄) δ: 8.24-6.63 (m, 8H), 4.47-4.36 (m, 2H), 4.01-3.75 (m, 5H), 2.45 (s, 3H), 1.54-1.41 (m, 9H).

### Example 39: 1-tert-butyl-N-[(5R)-8-[6-(1-methylpyrazol-4-yl)pyrazolo[1,5-a]pyrazin-4-yl]-2-(oxetan-3-yl)-1,3,4,5-tetrahydro-2-benzazepin-5-yl]triazole-4-carboxamide

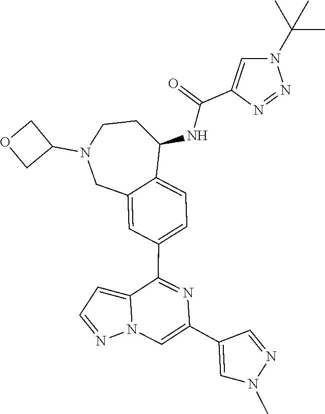 [SMILES: Cn1cc(-c2cn3nccc3c(-c3ccc4c(c3)CN(C3COC3)CC[C@H]4NC(=O)c3cn(C(C)(C)C)nn3)n2)cn1]

### 1. Synthesis of tert-butyl (R)-5-(1-(tert-butyl)-1H-1,2,3-triazole-4-carboxamido)-8-(6-(1-methyl-1H-pyrazol-4-yl)pyrazolo[1,5-a]pyrazin-4-yl)-1,3,4,5-tetrahydro-2H-benzo[c]azepine-2-carboxylate

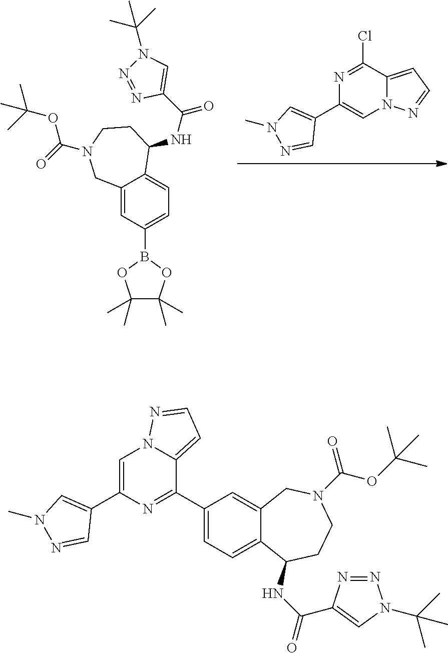

A mixture of tert-butyl (R)-5-(1-(tert-butyl)-1H-1,2,3-triazole-4-carboxamido)-8-(4,4,5,5-tetramethyl-1,3,2-dioxaborolan-2-yl)-1,3,4,5-tetrahydro-2H-benzo[c]azepine-2-carboxylate (WO2018/191577, Example 23, step 2, 500 mg, 926.8 μmol), 4-chloro-6-(1-methylpyrazol-4-yl)pyrazolo[1,5-a]pyrazine (Example 1, step 5, 259.9 mg, 1.11 mmol), K₂CO₃ (384.29 mg, 2.78 mmol), and Pd(dppf)Cl₂ (33.91 mg, 46.34 μmol) was dissolved in dioxane (4.94 mL) and water (1.24 mL). N2 was bubbled through the mixture for 5 min and the reaction was heated to 100° C. overnight. The cooled reaction was diluted with EtOAc and water, the layers separated, the organic layer was washed with brine and concentrated in vacuo. The material was used in the next step as crude assuming 100% yield. LCMS m/z=611.0 [M+H)]+. 2. Synthesis of (R)-1-(tert-butyl)-N-(8-(6-(1-methyl-1H-pyrazol-4-yl)pyrazolo[1,5-a]pyrazin-4-yl)-2,3,4,5-tetrahydro-1H-benzo[c]azepin-5-yl)-1H-1,2,3-triazole-4-carboxamide hydrochloride

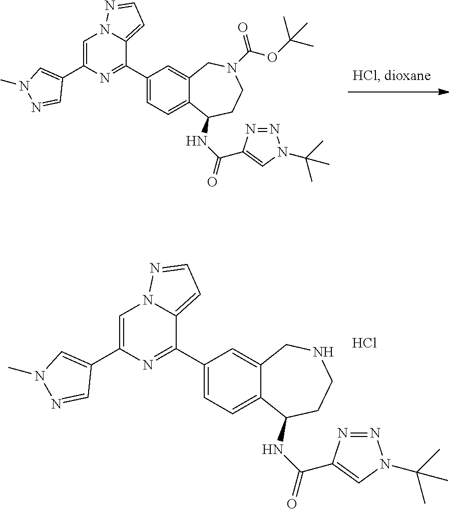

To a solution of tert-butyl (R)-5-(1-(tert-butyl)-1H-1,2,3-triazole-4-carboxamido)-8-(6-(1-methyl-1H-pyrazol-4-yl)pyrazolo[1,5-a]pyrazin-4-yl)-1,3,4,5-tetrahydro-2H-benzo[c]azepine-2-carboxylate (488 mg, 0.80 mmol) in dioxane (7.99 mL) was added HCl (4 M, 1.20 mL) and the reaction was stirred at RT overnight. The resulting solids were filtered off, washed with EtOAc and air dried to afford (R)-1-(tert-butyl)-N-(8-(6-(1-methyl-1H-pyrazol-4-yl)pyrazolo[1,5-a]pyrazin-4-yl)-2,3,4,5-tetrahydro-1H-benzo[c]azepin-5-yl)-1H-1,2,3-triazole-4-carboxamide hydrochloride (110 mg, 25.2% yield) as an orange solid. ¹H NMR (500 MHz, DMSO-d₆) δ: 9.41 (br d, J=7.94 Hz, 1H), 9.16 (s, 1H), 8.82 (s, 1H), 8.36 (s, 1H), 8.30-8.18 (m, 2H), 8.14 (s, 1H), 7.53 (br d, J=7.94 Hz, 1H), 7.31 (br d, J=2.44 Hz, 1H), 5.65 (t, J=1.00 Hz, 1H), 4.74-4.54 (m, 2H), 4.43-4.18 (m, 2H), 3.91 (s, 3H), 2.38-2.14 (m, 2H), 1.67 (s, 9H).

### 3. Synthesis of 1-tert-butyl-N-[(5R)-8-[6-(1-methylpyrazol-4-yl)pyrazolo[1,5-a]pyrazin-4-yl]-2-(oxetan-3-yl)-1,3,4,5-tetrahydro-2-benzazepin-5-yl]triazole-4-carboxamide

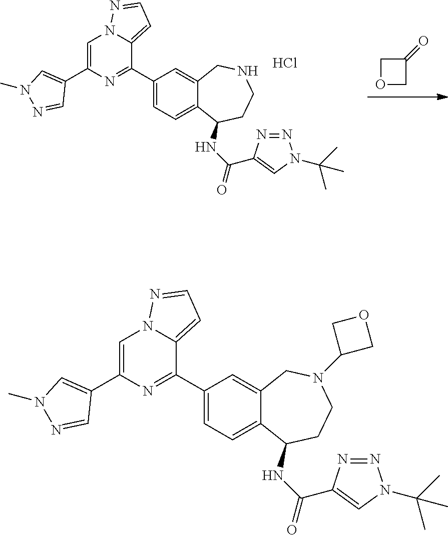

To a solution of (R)-1-(tert-butyl)-N-(8-(6-(1-methyl-1H-pyrazol-4-yl)pyrazolo[1,5-a]pyrazin-4-yl)-2,3,4,5-tetrahydro-1H-benzo[c]azepin-5-yl)-1H-1,2,3-triazole-4-carboxamide hydrochloride (55 mg, 96.95 μmol) in MeOH (1.94 mL) was added oxetan-3-one (34.9 mg, 485 μmol) and NaCNBH₃ (18.3 mg, 291 μmol) and the reaction mixture was stirred at RT overnight. The material was concentrated and purified by reverse phase purification (Method B2, 5-45% gradient) to afford (R)-1-(tert-butyl)-N-(8-(6-(1-methyl-1H-pyrazol-4-yl)pyrazolo[1,5-a]pyrazin-4-yl)-2-(oxetan-3-yl)-2,3,4,5-tetrahydro-1H-benzo[c]azepin-5-yl)-1H-1,2,3-triazole-4-carboxamide, (26.4 mg, 45.7% yield) as a pale yellow solid. LCMS m/z=567.0 [M+H]⁺. ¹H NMR (500 MHz, DMSO-d₆) δ ppm 9.49 (s, 1H), 9.19 (s, 1H), 8.81 (s, 1H), 8.34 (s, 1H), 8.30-8.18 (m, 2H), 8.14 (s, 1H), 7.56 (d, J=1.00 Hz, 1H), 7.21 (br d, J=1.83 Hz, 1H), 5.71 (s, 1H), 5.01-4.45 (m, 8H), 3.92 (s, 2H), 3.54 (s, 1H), 2.43-2.25 (m, 2H), 1.68 (s, 9H)

### Example 40: (R)-1-(tert-butyl)-N-(8-(6-(1-methyl-1H-pyrazol-4-yl)pyrazolo[1,5-a]pyrazin-4-yl)-2-(2,2,2-trifluoroethyl)-2,3,4,5-tetrahydro-1H-benzo[c]azepin-5-yl)-1H-1,2,3-triazole-4-carboxamide

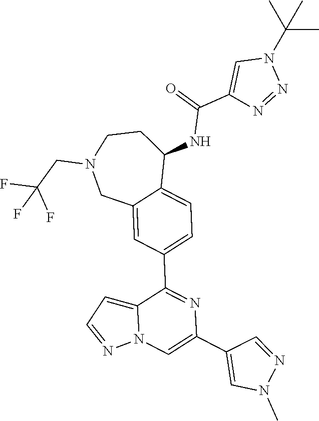

### 1. Synthesis of (R)-1-(tert-butyl)-N-(8-(6-(1-methyl-1H-pyrazol-4-yl)pyrazolo[1,5-a]pyrazin-4-yl)-2-(2,2,2-trifluoroethyl)-2,3,4,5-tetrahydro-1H-benzo[c]azepin-5-yl)-1H-1,2,3-triazole-4-carboxamide

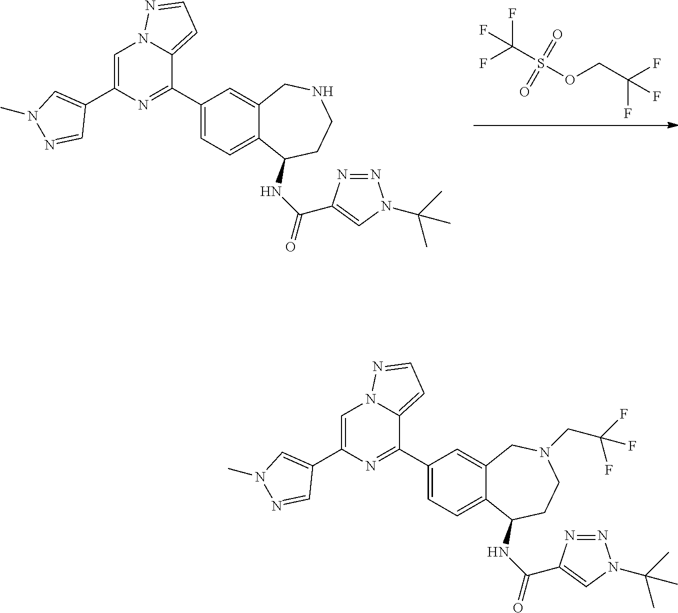

To a solution of (R)-1-(tert-butyl)-N-(8-(6-(1-methyl-1H-pyrazol-4-yl)pyrazolo[1,5-a]pyrazin-4-yl)-2,3,4,5-tetrahydro-1H-benzo[c]azepin-5-yl)-1H-1,2,3-triazole-4-carboxamide hydrochloride (Example 39, step 2, 55.0 mg, 101 μmol) in MeCN (1.01 mL) was added 2,2,2-trifluoroethyl trifluoromethanesulfonate (46.7 mg, 201 μmol) and K₂CO₃ (41.7 mg, 302 μmol) and the reaction mixture was stirred under N2 at 80° C. overnight. The material was filtered through Celite®, concentrated, and purified by reverse phase HPLC (Method B2, 5-45% gradient) to afford (R)-1-(tert-butyl)-N-(8-(6-(1-methyl-1H-pyrazol-4-yl)pyrazolo[1,5-a]pyrazin-4-yl)-2-(2,2,2-trifluoroethyl)-2,3,4,5-tetrahydro-1H-benzo[c]azepin-5-yl)-1H-1,2,3-triazole-4-carboxamide, 16.9 mg (27.8% yield) as brown solid. LCMS m/z=593.0 [M+H]⁺. ¹H NMR (500 MHz, DMSO-d₆) δ ppm 9.14 (s, 1H), 9.09 (br d, J=7.94 Hz, 1H), 8.77 (s, 1H), 8.33 (s, 1H), 8.20 (d, J=2.44 Hz, 1H), 8.12 (s, 1H), 8.03 (dd, J=7.94, 1.83 Hz, 1H), 7.96 (d, J=1.83 Hz, 1H), 7.46 (br d, J=8.55 Hz, 1H), 7.17 (dd, J=2.44, 1.22 Hz, 1H), 5.51 (t, J=1.00 Hz, 1H), 4.35 (br d, J=15.26 Hz, 2H), 4.10 (br d, J=15.26 Hz, 2H), 3.91 (s, 3H), 3.26-3.21 (m, 2H), 2.20-2.12 (m, 2H), 1.67 (s, 9H)

### Example 41. 5-(tert-butyl)-N-((4-methyl-1-(6-(1-methyl-1H-pyrazol-4-yl)pyrazolo[1,5-a]pyrazin-4-yl)piperidin-4-yl)methyl)-1,2,4-oxadiazole-3-carboxamide

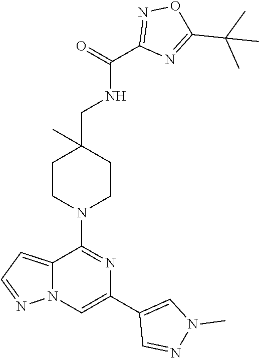 [SMILES: Cn1cc(-c2cn3nccc3c(N3CCC(C)(CNC(=O)c4noc(C(C)(C)C)n4)CC3)n2)cn1]

### 1. Preparation of tert-butyl ((4-methyl-1-(6-(1-methyl-1H-pyrazol-4-yl)pyrazolo[1,5-a]pyrazin-4-yl)piperidin-4-yl)methyl)carbamate

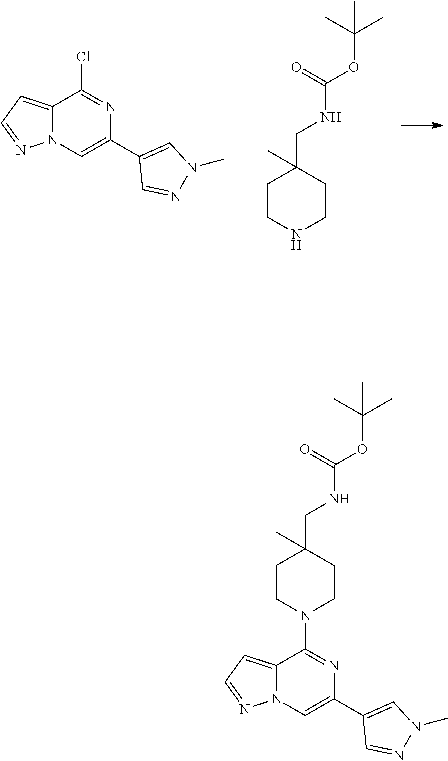

tert-Butyl N-((4-methyl-4-piperidyl)methyl)carbamate (296.83 mg, 1.30 mmol) followed by Cs₂CO₃ (521.31 mg, 1.60 mmol) were added to a solution of 4-chloro-6-(1-methyl-1H-pyrazol-4-yl)pyrazolo[1,5-a]pyrazine (Example 1, step 5, 233.66 mg, 1.00 mmol) in DMF (3.0 mL) and the reaction stirred at RT overnight. The reaction was diluted with water (10 mL) and extracted with EtOAc (2×10 mL). The combined organic phases were dried over Na₂SO₄, filtered and evaporated to dryness. The residual material was purified by silica gel column chromatography (1:1 heptane:EtOAc) to give tert-butyl ((4-methyl-1-(6-(1-methyl-1H-pyrazol-4-yl)pyrazolo[1,5-a]pyrazin-4-yl)piperidin-4-yl)methyl)carbamate (430 mg, 90% yield) as a dark brown gum. LCMS m/z=426.1 [M+H]⁺.

### 2. Preparation of(4-methyl-1-(6-(1-methyl-1H-pyrazol-4-yl)pyrazolo[1,5-a]pyrazin-4-yl)piperidin-4-yl)methanamine

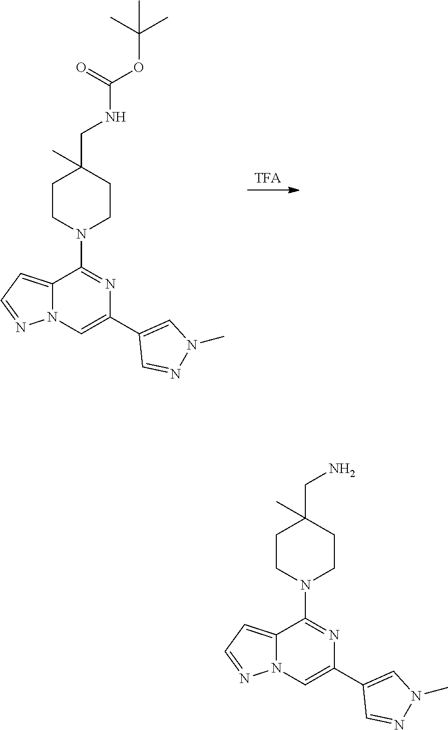 [SMILES: Cn1cc(-c2cn3nccc3c(N3CCC(C)(CNC(=O)OC(C)(C)C)CC3)n2)cn1 ; Cn1cc(-c2cn3nccc3c(N3CCC(C)(CN)CC3)n2)cn1 ; "TFA"]

To a solution of tert-butyl ((4-methyl-1-(6-(1-methyl-1H-pyrazol-4-yl)pyrazolo[1,5-a]pyrazin-4-yl)piperidin-4-yl)methyl)carbamate (429.79 mg, 1.01 mmol) in DCM (3.0 mL) was added TFA (2.30 g, 20.20 mmol) and the reaction stirred at RT overnight. The mixture was diluted with MeOH and purified on a 5 g SCX column where the product eluted with 2M NH₃-MeOH to give (4-methyl-1-(6-(1-methyl-1H-pyrazol-4-yl)pyrazolo[1,5-a]pyrazin-4-yl)piperidin-4-yl)methanamine (260 mg, 71.2% yield) as a beige solid. LCMS m/z=326.1 [M+H]⁺.

### 3. Preparation of 5-(tert-butyl)-N-((4-methyl-1-(6-(1-methyl-1H-pyrazol-4-yl)pyrazolo[1,5-a]pyrazin-4-yl)piperidin-4-yl)methyl)-1,2,4-oxadiazole-3-carboxamide

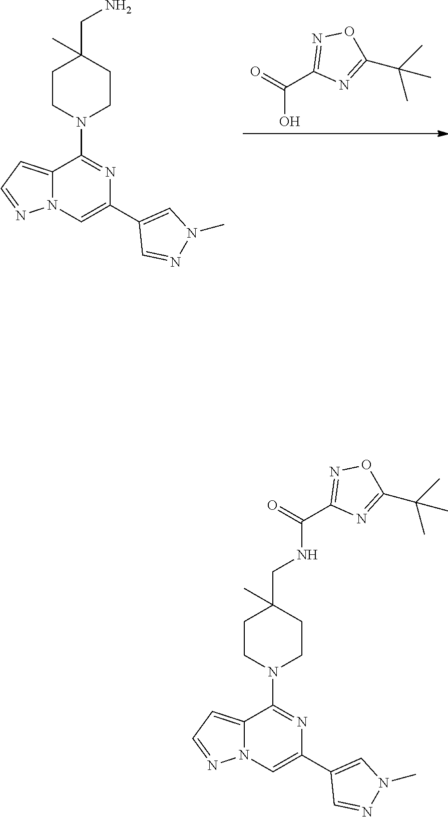 [SMILES: Cn1cc(-c2cn3nccc3c(N3CCC(C)(CN)CC3)n2)cn1 ; CC(C)(C)c1nc(C(=O)O)no1 ; Cn1cc(-c2cn3nccc3c(N3CCC(C)(CNC(=O)c4noc(C(C)(C)C)n4)CC3)n2)cn1]

DIPEA (24 mg, 185 μmol) was added to a mixture of (4-methyl-1-(6-(1-methyl-1H-pyrazol-4-yl)pyrazolo[1,5-a]pyrazin-4-yl)piperidin-4-yl)methanamine (40.0 mg, 122.92 μmol) and 5-tert-butyl-1,2,4-oxadiazole-3-carboxylic acid (23 mg, 135.2 μmol) in DMF (1 mL) and the mixture stirred for 5 mins at RT. T3P® (120 mg, 185 μmol, 50% purity) was added and the reaction stirred at RT overnight. The mixture was diluted with EtOAc (3 mL) and water (3 mL) and the mixture stirred vigorously. The aqueous phase was discarded, the remaining organic phase dried over Na₂SO₄, and filtered. The filtrate was evaporated, the residual material was re-dissolved in DMSO and the product was purified by prep-HPLC (Method A, 5-60% gradient). LCMS mz=478.1 [M+H]⁺. ¹H NMR (DMSO-d₆) δ: 8.96-8.87 (m, 1H), 8.44 (s, 1H), 8.14 (s, 1H), 7.94 (s, 1H), 7.93 (d, J=2.4 Hz, 1H), 6.97-6.88 (m, 1H), 4.08-3.98 (m, 2H), 3.87 (s, 3H), 3.69-3.59 (m, 2H), 3.31-3.25 (m, 2H), 1.66-1.57 (m, 2H), 1.49-1.35 (m, 11H), 1.02 (s, 3H).

### Example 42. 5-(tert-butyl)-N-((3-methyl-1-(6-(1-methyl-1H-pyrazol-4-yl)pyrazolo[1,5-a]pyrazin-4-yl)pyrrolidin-3-yl)methyl)-1,2,4-oxadiazole-3-carboxamide

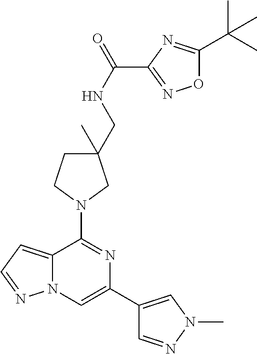 [SMILES: Cn1cc(-c2cn3nccc3c(N3CCC(C)(CNC(=O)c4noc(C(C)(C)C)n4)C3)n2)cn1]

### 1. Preparation of tert-butyl ((3-methyl-1-(6-(1-methyl-1H-pyrazol-4-yl)pyrazolo[1,5-a]pyrazin-4-yl)pyrrolidin-3-yl)methyl)carbamate

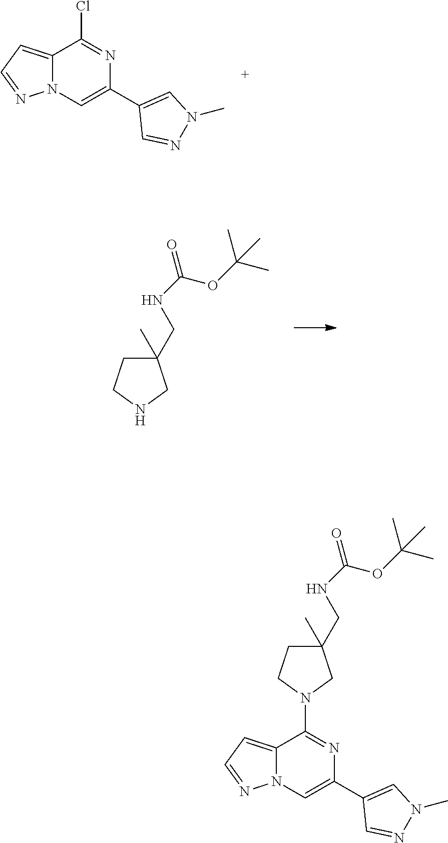 [SMILES: Cn1cc(-c2cn3nccc3c(Cl)n2)cn1 ; CC1(CNC(=O)OC(C)(C)C)CCNC1 ; Cn1cc(-c2cn3nccc3c(N3CCC(C)(CNC(=O)OC(C)(C)C)C3)n2)cn1]

tert-Butyl ((3-methyl-1-(6-(1-methyl-1H-pyrazol-4-yl)pyrazolo[1,5-a]pyrazin-4-yl)pyrrolidin-3-yl)methyl)carbamate was obtained (300 mg, 65.6% yield) as a dark brown gum, from 4-chloro-6-(1-methylpyrazol-4-yl)pyrazolo[1,5-a]pyrazine (Example 1, step 5) and tert-butyl N-((3-methylpyrrolidin-3-yl)methyl)carbamate, following a similar method to that described in Example 41, step 1. LCMS m/z=412.1 [M+H]⁺.

### 2. Preparation of (3-methyl-1-(6-(1-methyl-1H-pyrazol-4-yl)pyrazolo[1,5-a]pyrazin-4-yl)pyrrolidin-3-yl)methanamine

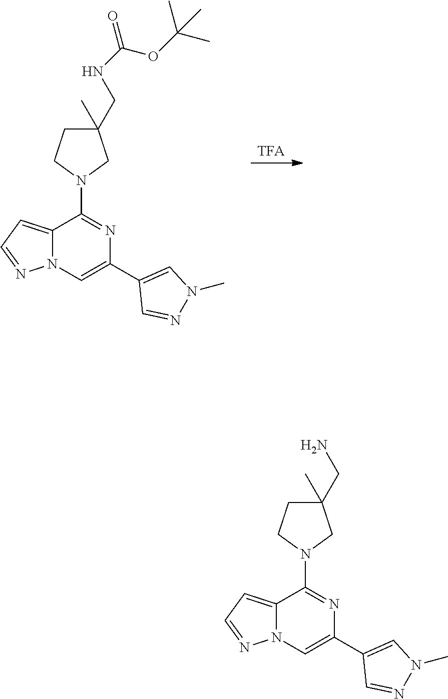 [SMILES: Cn1cc(-c2cn3nccc3c(N3CCC(C)(CNC(=O)OC(C)(C)C)C3)n2)cn1 ; Cn1cc(-c2cn3nccc3c(N3CCC(C)(CN)C3)n2)cn1 ; "TFA"]

(3-Methyl-1-(6-(1-methyl-1H-pyrazol-4-yl)pyrazolo[1,5-a]pyrazin-4-yl)pyrrolidin-3-yl)methanamine was obtained (210 mg, 83.2% yield) as a pale yellow solid, from tert-butyl ((3-methyl-1-(6-(1-methyl-1H-pyrazol-4-yl)pyrazolo[1,5-a]pyrazin-4-yl)pyrrolidin-3-yl)methyl)carbamate following a similar method to that described in Example 41, step 2. LCMS m/z=312.1 [M+H]⁺.

### 3. Preparation of 5-(tert-butyl)-N-((3-methyl-1-(6-(1-methyl-1H-pyrazol-4-yl)pyrazolo[1,5-a]pyrazin-4-yl)pyrrolidin-3-yl)methyl)-1,2,4-oxadiazole-3-carboxamide

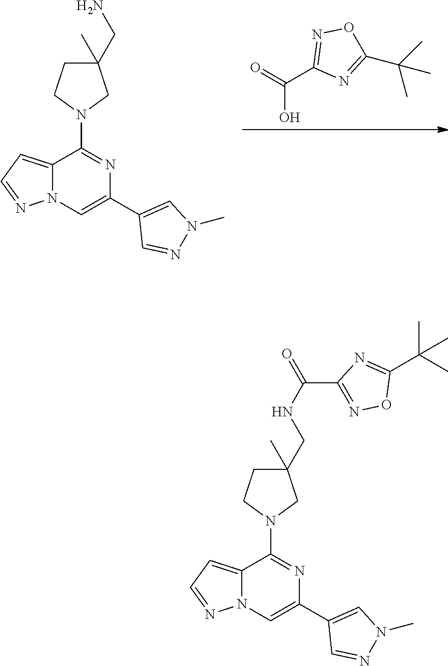 [SMILES: Cn1cc(-c2cn3nccc3c(N3CCC(C)(CN)C3)n2)cn1 ; CC(C)(C)c1nc(C(=O)O)no1 ; Cn1cc(-c2cn3nccc3c(N3CCC(C)(CNC(=O)c4noc(C(C)(C)C)n4)C3)n2)cn1]

5-(tert-Butyl)-N-((3-methyl-1-(6-(1-methyl-1H-pyrazol-4-yl)pyrazolo[1,5-a]pyrazin-4-yl)pyrrolidin-3-yl)methyl)-1,2,4-oxadiazole-3-carboxamide was obtained from (3-methyl-1-(6-(1-methyl-1H-pyrazol-4-yl)pyrazolo[1,5-a]pyrazin-4-yl)pyrrolidin-3-yl)methanamine and 5-tert-butyl-1,2,4-oxadiazole-3-carboxylic acid following the method used in Example 41, step 3. LCMS m/z=464.0 [M+H]⁺. ¹H NMR (DMSO-d₆) δ: 9.11 (t, J=6.4 Hz, 1H), 8.29 (s, 1H), 8.10 (s, 1H), 7.92 (s, 1H), 7.89 (d, J=2.4 Hz, 1H), 7.04-6.92 (m, 1H), 3.86 (s, 5H), 3.64-3.39 (m, 2H), 2.09-1.92 (m, 1H), 1.84-1.67 (m, 1H), 1.41 (s, 9H), 1.13 (s, 3H).

### Examples 43 to 51

The compounds in the following table were prepared from (4-methyl-1-(6-(1-methylpyrazol-4-yl)pyrazolo[1,5-a]pyrazin-4-yl])-4-piperidyl)methanamine (Example 41, step 2) or (3-methyl-1-(6-(1-methyl-1H-pyrazol-4-yl)pyrazolo[1,5-a]pyrazin-4-yl)pyrrolidin-3-yl)methanamine (Example 42, step 2) and the appropriate carboxylic acid, following a similar method to that used for Example 41, step 3.

| | |
|---|---|
| Example | |
| No | Name/Structure/Carboxylic acid/Data |

Using (4-methyl-1-(6-(1-methylpyrazol-4-yl)pyrazolo[1,5-a]pyrazin-4-yl])-4-
piperidyl)methanamine (Example 41, step 2)

| | |
|---|---|
| 43 | 2-(tert-butyl)-N-((4-methyl-1-(6-(1-methyl-1H-pyrazol-4-yl)pyrazolo[1,5- a]pyrazin-4-yl)piperidin-4-yl)methyl)-2H-tetrazole-5-carboxamide 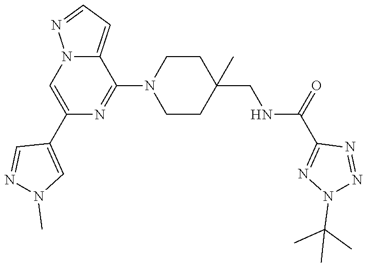 [SMILES: Cn1cc(-c2cn3nccc3c(N3CCC(C)(CNC(=O)c4nnn(C(C)(C)C)n4)CC3)n2)cn1] Carboxylic acid: 2-tert-butyltetrazole-5-carboxylic acid Prep-HPLC (Method A, 5-60% gradient) LCMS m/z = 478.1 [M + H]⁺. 1H NMR (DMSO-d6) 8: 8.95 (s, 1H), 8.43 (s, 1H), 8.14 (s, 1H), 7.94 (s, 1H), 7.93 (d, J = 2.4 Hz, 1H), 6.95-6.90 (m, 1H), 4.09-3.99 (m, 2H), 3.86 (s, 3H), 3.70-3.60 (m, 2H), 1.79-1.68 (m, 9H), 1.68-1.57 (m, 2H), 1.51-1.38 (m, 2H), 1.03 (s, 3H). |
| 44 | 1-(tert-butyl)-N-((4-methyl-1-(6-(1-methyl-1H-pyrazol-4-yl)pyrazolo[1,5- a]pyrazin-4-yl)piperidin-4-yl)methyl)-1H-1,2,3-triazole-4-carboxamide 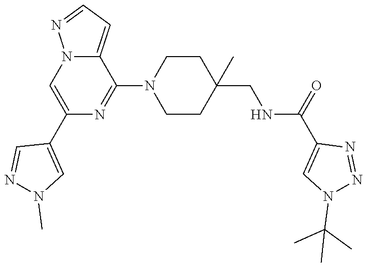 [SMILES: Cn1cc(-c2cn3nccc3c(N3CCC(C)(CNC(=O)c4cn(C(C)(C)C)nn4)CC3)n2)cn1] Carboxylic acid: 1-tert-butyltriazole-4-carboxylic acid Prep-HPLC (Method A, 5-60% gradient) LCMS m/z = 477.1 [M + H]⁺ ¹H NMR (DMSO-d₆) δ: 8.66 (s, 1H), 8.43 (s, 1H), 8.42-8.35 (m, 1H), 8.14 (s, 1H), 7.94 (s, 1H), 7.93 (d, J = 2.4 Hz, 1H), 6.91 (d, J = 2.4 Hz, 1H), 4.03 (ddd, J = 13.3, 6.6, 4.0 Hz, 2H), 3.87 (s, 3H), 3.70-3.61 (m, 2H), 1.62 (s, 11H), 1.49-1.37 (m, 2H), 1.02 (s, 3H). |
| 45 | 2-(tert-butyl)-N-((4-methyl-1-(6-(1-methyl-1H-pyrazol-4-yl)pyrazolo[1,5- a]pyrazin-4-yl)piperidin-4-yl)methyl)oxazole-5-carboxamide 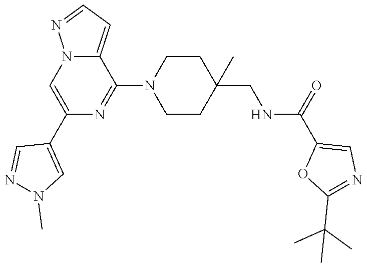 [SMILES: Cn1cc(-c2cn3nccc3c(N3CCC(C)(CNC(=O)c4cnc(C(C)(C)C)o4)CC3)n2)cn1] Carboxylic acid: 2-(tert-butyl)oxazole-5-carboxylic acid Prep-HPLC (Method C2, 5-60% gradient) LCMS m/z = 477.1 [M + H]⁺. ¹H NMR (DMSO-d₆) δ: 8.43 (s, 2H), 8.14 (s, 1H), 7.94 (s, 1H), 7.93 (d, J = 2.4 Hz, 1H), 7.67 (s, 1H), 6.92 (d, J = 2.4 Hz, 1H), 4.09- 3.98 (m, 2H), 3.87 (s, 3H), 3.70-3.60 (m, 2H), 3.29-3.20 (m, 2H), 1.65-1.54 (m, 2H), 1.49-1.39 (m, 2H), 1.38-1.29 (m, 9H), 1.02 (s, 3H). |
| 46 | N-((4-methyl-1-(6-(1-methyl-1H-pyrazol-4-yl)pyrazolo[1,5-a]pyrazin-4- yl)piperidin-4-yl)methyl)-5-(1-methylcyclopropyl)-1,2,4-oxadiazole-3- carboxamide 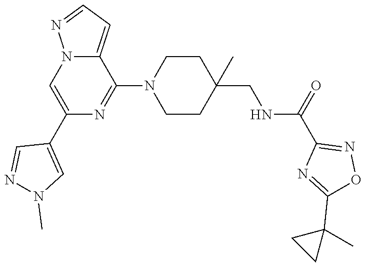 [SMILES: Cn1cc(-c2cn3nccc3c(N3CCC(C)(CNC(=O)c4noc(C5(C)CC5)n4)CC3)n2)cn1] Carboxylic acid: 5-(1-methylcyclopropyl)-1,2,4-oxadiazole-3-carboxylic acid Prep HPLC (Method A, 5-60% gradient) LCMS m/z = 476.1 [M + H]⁺. ¹H NMR (DMSO-d₆) δ: 8.85 (s, 1H), 8.44 (s, 1H), 8.14 (s, 1H), 7.94 (s, 1H), 7.93 (d, J = 2.4 Hz, 1H), 6.96-6.90 (m, 1H), 4.08-3.98 (m, 2H), 3.86 (s, 3H), 3.67-3.59 (m, 2H), 3.29-3.23 (m, 2H), 1.65-1.56 (m, 2H), 1.53 (s, 3H), 1.47-1.39 (m, 2H), 1.36 (d, J = 2.4 Hz, 2H), 1.15 (d, J = 3.1 Hz, 2H), 1.01 (s, 3H). |
| 47 | 3-(tert-butyl)-N-((4-methyl-1-(6-(1-methyl-1H-pyrazol-4-yl)pyrazolo[1,5- a]pyrazin-4-yl)piperidin-4-yl)methyl)-1,2,4-oxadiazole-5-carboxamide 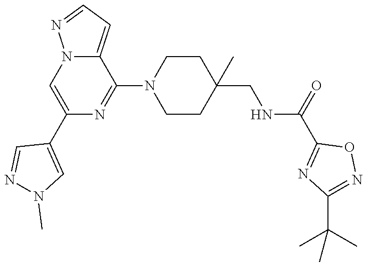 [SMILES: Cn1cc(-c2cn3nccc3c(N3CCC(C)(CNC(=O)c4nc(C(C)(C)C)no4)CC3)n2)cn1] Carboxylic acid: 3-tert-butyl-1,2,4-oxadiazole-5-carboxylic acid Prep-HPLC (Method C2, 5-60% gradient) LCMS m/z = 478.1 [M + H]⁺. ¹H NMR (DMSO-d₆) δ: 9.36-9.29 (m, 1H), 8.44 (s, 1H), 8.14 (s, 1H), 7.95 (s, 1H), 7.93 (d, J = 2.4 Hz, 1H), 6.96-6.90 (m, 1H), 4.08-3.98 (m, 2H), 3.87 (s, 3H), 3.68-3.60 (m, 2H), 3.31-3.25 (m, 2H), 1.63 (ddd, J = 13.3, 9.3, 3.7 Hz, 2H), 1.46 (br d, J = 2.4 Hz, 2H), 1.40-1.30 (m, 9H), 1.03 (s, 3H) |

Using (3-methyl-1-(6-(1-methyl-1H-pyrazol-4-yl)pyrazolo[1,5-a]pyrazin-4-
yl)pyrrolidin-3-yl)methanamine (Example 42, step 2)

| | |
|---|---|
| 48 | 2-(tert-butyl)-N-((3-methyl-1-(6-(1-methyl-1H-pyrazol-4-yl)pyrazolo[1,5- a]pyrazin-4-yl)pyrrolidin-3-yl)methyl)-2H-tetrazole-5-carboxamide 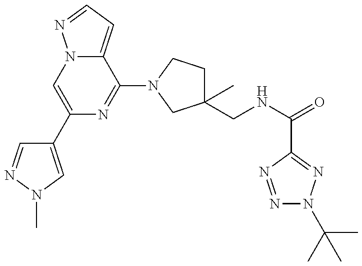 [SMILES: Cn1cc(-c2cn3nccc3c(N3CCC(C)(CNC(=O)c4nnn(C(C)(C)C)n4)C3)n2)cn1] Carboxylic acid: 2-tert-butyltetrazole-5-carboxylic acid Prep-HPLC (Method A, 5-60% gradient) LCMS m/z = 464.0 [M + H]⁺. 1H NMR (DMSO-d6) 8: 9.18 (t, J = 6.4 Hz, 1H), 8.28 (s, 1H), 8.09 (s, 1H), 7.92 (s, 1H), 7.89 (d, J = 2.4 Hz, 1H), 7.05-6.93 (m, 1H), 4.08-3.78 (m, 5H), 3.61-3.40 (m, 2H), 2.11-1.93 (m, 1H), 1.82-1.60 (m, 10H), 1.15 (s, 3H). |
| 49 | 1-(tert-butyl)-N-((3-methyl-1-(6-(1-methyl-1H-pyrazol-4-yl)pyrazolo[1,5- a]pyrazin-4-yl)pyrrolidin-3-yl)methyl)-1H-1,2,3-triazole-4-carboxamide 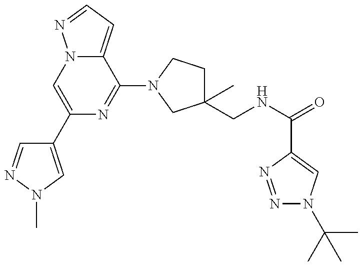 [SMILES: Cn1cc(-c2cn3nccc3c(N3CCC(C)(CNC(=O)c4cn(C(C)(C)C)nn4)C3)n2)cn1] Carboxylic acid: 1-tert-butyltriazole-4-carboxylic acid Prep-HPLC (Method A, 5-60% gradient) LCMS m/z = 463.0 [M + H]⁺. ¹H NMR (DMSO-d₆) δ: 8.66 (s, 1H), 8.28 (s, 1H), 8.10 (s, 1H), 7.92 (s, 1H), 7.89 (d, J = 2.4 Hz, 1H), 7.01-6.93 (m, 1H), 3.86 (s, 5H), 2.06-1.95 (m, 1H), 1.80-1.69 (m, 1H), 1.62 (s, 9H), 1.13 (s, 3H). |
| 50 | 2-(tert-butyl)-N-((3-methyl-1-(6-(1-methyl-1H-pyrazol-4-yl)pyrazolo[1,5- a]pyrazin-4-yl)pyrrolidin-3-yl)methyl)oxazole-5-carboxamide 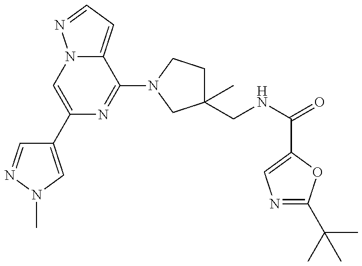 [SMILES: Cn1cc(-c2cn3nccc3c(N3CCC(C)(CNC(=O)c4cnc(C(C)(C)C)o4)C3)n2)cn1] Carboxylic acid: 2-tert-butyloxazole-5-carboxylic acid Prep HPLC (Method C2, 5-60% gradient) LCMS m/z = 463.0 [M + H]⁺ ¹H NMR (DMSO-d₆) δ: 8.63-8.55 (m, 1H), 8.33- 8.26 (m, 1H), 8.13-8.07 (m, 1H), 7.93-7.91 (m, 1H), 7.90-7.87 (m, 1H), 7.66 (s, 1H), 6.97-6.94 (m, 1H), 3.86 (s, 7H), 3.32-3.24 (m, 1H), 2.07-1.92 (m, 2H), 1.82-1.67 (m, 2H), 1.34 (s, 9H), 1.14 (s, 3H). |
| 51 | N-((3-methyl-1-(6-(1-methyl-1H-pyrazol-4-yl)pyrazolo[1,5-a]pyrazin-4- yl)pyrrolidin-3-yl)methyl)-5-(1-methylcyclopropyl)-1,2,4-oxadiazole-3- carboxamide 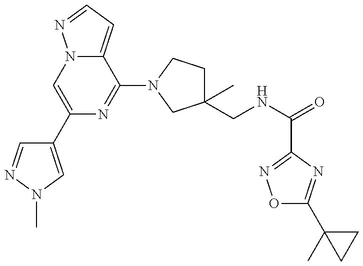 [SMILES: Cn1cc(-c2cn3nccc3c(N3CCC(C)(CNC(=O)c4noc(C5(C)CC5)n4)C3)n2)cn1] Carboxylic acid: 5-(1-methylcyclopropyl)-1,2,4-oxadiazole-3-carboxylic acid Prep-HPLC (Method C2, 5-60% gradient) LCMS m/z = 462.0 [M + H]⁺ ¹H NMR (DMSO-d₆) δ: 9.05 (s, 1H), 8.29 (s, 1H), 8.09 (s, 1H), 7.92 (s, 1H), 7.91-7.85 (m, 1H), 6.97-6.93 (m, 1H), 3.86 (s, 5H), 2.06-1.93 (m, 1H), 1.81-1.69 (m, 1H), 1.52 (s, 3H), 1.40-1.31 (m, 2H), 1.12 (s, 5H). |

### Example 52: 5-(tert-butyl)-N-(6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide

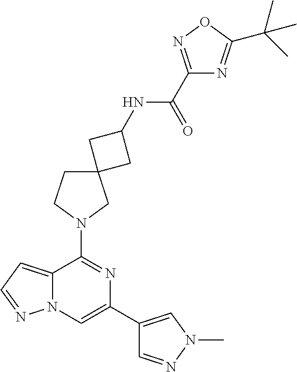 [SMILES: Cn1cc(-c2cn3nccc3c(N3CCC4(CC(NC(=O)c5noc(C(C)(C)C)n5)C4)C3)n2)cn1]

### 1. Preparation of tert-butyl 2-(5-(tert-butyl)-1,2,4-oxadiazole-3-carboxamido)-6-azaspiro[3.4]octane-6-carboxylate

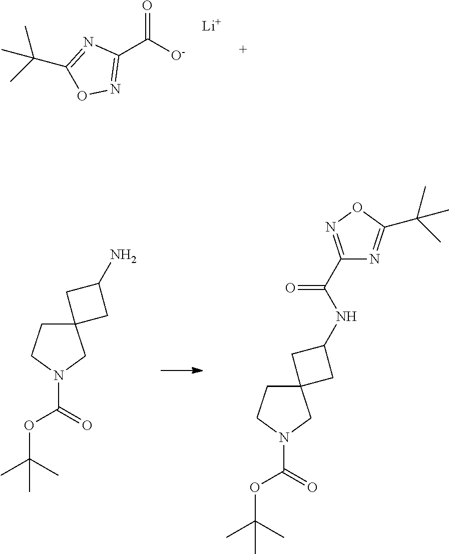 [SMILES: CC(C)(C)c1nc(C(=O)[O-])no1 ; [Li+] ; CC(C)(C)OC(=O)N1CCC2(CC(N)C2)C1 ; CC(C)(C)OC(=O)N1CCC2(CC(NC(=O)c3noc(C(C)(C)C)n3)C2)C1]

tert-Butyl 2-amino-6-azaspiro[3.4]octane-6-carboxylate (160.0 mg, 706.96 μmol), followed by T3P® (674.83 mg, 1.06 mmol, 50% purity) were added to a solution of lithium 5-(tert-butyl)-1,2,4-oxadiazole-3-carboxylate (373.49 mg, 2.12 mmol) in DMF (1.0 mL) and the reaction warmed to 50° C. and stirred overnight. The mixture was diluted with water (3 mL) and the product was extracted with EtOAc (2×3 mL). The combined organic phases were washed with brine (2 mL), dried over Na₂SO₄ and filtered. The filtrate was evaporated to dryness and the material was purified on a 5 g SPE column eluting with heptane:EtOAc, to give tert-butyl 2-(5-(tert-butyl)-1,2,4-oxadiazole-3-carboxamido)-6-azaspiro[3.4]octane-6-carboxylate as a white solid. LCMS m/z=313.1 [M-tBu+H]⁺

### 2. Preparation of 5-(tert-butyl)-N-(6-azaspiro[3.4]octan-2-yl)-1,2,4-oxadiazole-3-carboxamide trifluoroacetate

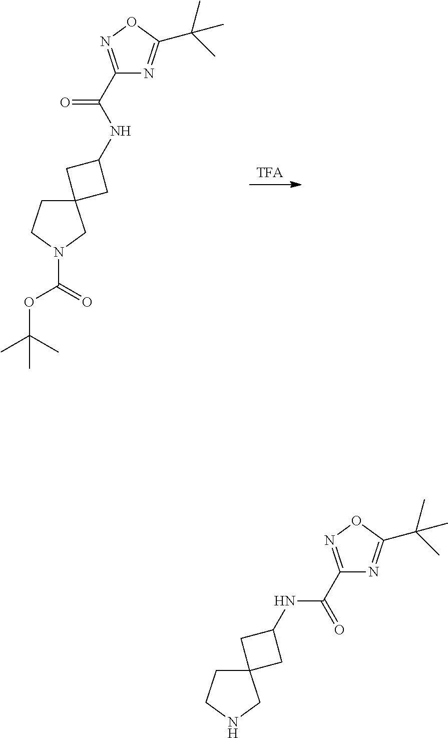 [SMILES: CC(C)(C)OC(=O)N1CCC2(CC(NC(=O)c3noc(C(C)(C)C)n3)C2)C1 ; CC(C)(C)c1nc(C(=O)NC2CC3(CCNC3)C2)no1 ; "TFA"]

To a solution of tert-butyl 2-(5-(tert-butyl)-1,2,4-oxadiazole-3-carboxamido)-6-azaspiro[3.4]octane-6-carboxylate (180.0 mg, 475.61 μmol) in DCM (3.0 mL) was added TFA (1.08 g, 9.51 mmol) and the reaction stirred at RT overnight. The mixture was diluted with MeOH (5.0 mL) and purified on a 5 g SCX column. The desired compound eluted with TEA/MeOH (1 mL/25 mL) to give 5-(tert-butyl)-N-(6-azaspiro[3.4]octan-2-yl)-1,2,4-oxadiazole-3-carboxamide trifluoroacetate (90.0 mg) as a colorless gum. LCMS m/z=279.1 [M+H]⁺.

### 3. Preparation of 5-(tert-butyl)-N-(6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide

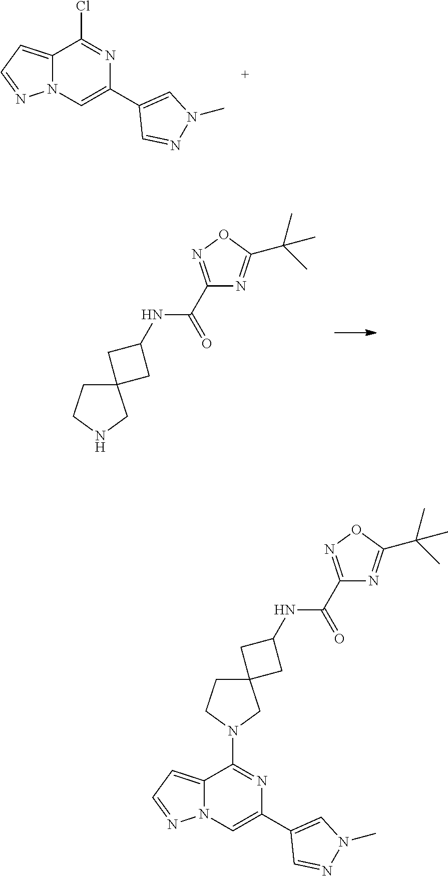 [SMILES: Cn1cc(-c2cn3nccc3c(Cl)n2)cn1 ; CC(C)(C)c1nc(C(=O)NC2CC3(CCNC3)C2)no1 ; Cn1cc(-c2cn3nccc3c(N3CCC4(CC(NC(=O)c5noc(C(C)(C)C)n5)C4)C3)n2)cn1]

N-(6-azaspiro[3.4]octan-2-yl)-5-tert-butyl-1,2,4-oxadiazole-3-carboxamide (42.89 mg, 154.07 μmol), followed by Cs₂CO₃ (62.75 mg, 192.58 μmol) were added to a solution of 4-chloro-6-(1-methylpyrazol-4-yl)pyrazolo[1,5-a]pyrazine (Example 1, step 5, 30.0 mg, 128.39 μmol) in DMF (2.0 mL) and the reaction stirred at RT overnight. The reaction was diluted with water (10 mL) and extracted with EtOAc (2×15 mL). The combined organic phases were dried over Na₂SO₄, filtered and evaporated to dryness. The residual material was re-dissolved in DMSO and the product purified by prep-HPLC (Method A, 5-60% gradient) to give 5-(tert-butyl)-N-(6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide. LCMS m/z=476.1 [M+H]⁺. ¹H NMR (DMSO-d₆) δ: 9.21 (dd, J=19.2, 7.6 Hz, 2H), 8.29 (s, 1H), 8.18-8.06 (m, 1H), 7.98-7.91 (m, 1H), 7.89 (d, J=1.8 Hz, 1H), 7.04 (s, 1H), 6.96 (d, J=2.4 Hz, 1H), 4.63-4.44 (m, 1H), 3.87 (d, J=1.8 Hz, 8H), 2.33 (br s, 6H), 2.11 (s, 2H), 2.06-1.98 (m, 1H), 1.43 (d, J=1.8 Hz, 9H).

### Examples 53 and 54. 5-(tert-butyl)-N-((2r,4s)-6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide and 5-(tert-butyl)-N-((2s,4r)-6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide

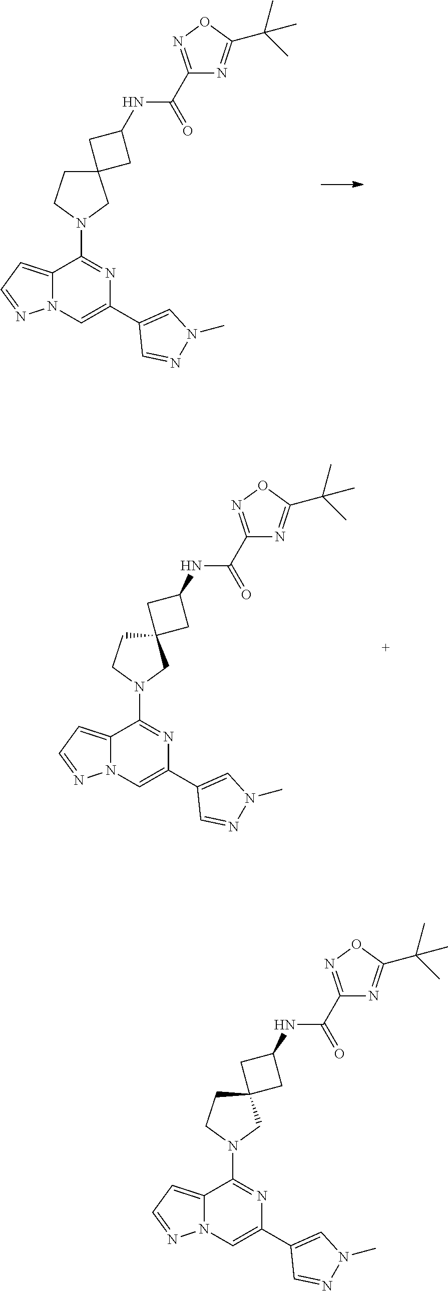 [SMILES: Cn1cc(-c2cn3nccc3c(N3CCC4(CC(NC(=O)c5noc(C(C)(C)C)n5)C4)C3)n2)cn1 ; Cn1cc(-c2cn3nccc3c(N3CC[C@]4(C3)C[C@@H](NC(=O)c3noc(C(C)(C)C)n3)C4)n2)cn1 ; Cn1cc(-c2cn3nccc3c(N3CC[C@]4(C3)C[C@H](NC(=O)c3noc(C(C)(C)C)n3)C4)n2)cn1]

### [Stereochemistry Arbitrarily Assigned]

5-(tert-Butyl)-N-(6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide was separated by SFC using a LUX Cellulose-4 LC 30×250 mm, 3 um column, eluting with 45% MeOH w/0.1% DEA in CO₂ (flow rate: 100 mL/min, ABPR 120bar, MBPR 40 psi, column temp 40 deg C.) to give, Peak 1: 5-(tert-butyl)-N-((2r,4s)-6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide. LCMS m/z=476.1 [M+H]⁺. ¹H NMR (CDCl₃) δ: 8.00 (s, 1H), 7.85 (s, 3H), 7.22-7.10 (m, 1H), 6.79-6.72 (m, 1H), 4.72 (br d, J=8.0 Hz, 1H), 4.02-3.96 (m, 5H), 3.93 (t, J=6.9 Hz, 2H), 2.69-2.56 (m, 2H), 2.19 (br d, J=3.5 Hz, 3H), 2.15-2.06 (m, 3H), 1.48 (s, 10H), 1.21-1.09 (m, 1H) and Peak 2: 5-(tert-butyl)-N-((2s,4r)-6-(6-(1-methyl-1H-pyrazol-4-yl)pyrazolo[1,5-a]pyrazin-4-yl)-6-azaspiro[3.4]octan-2-yl)-1,2,4-oxadiazole-3-carboxamide. LCMS m/z=476.1 [M+H]⁺. ¹H NMR (CDCl₃) δ: 7.98 (d, J=0.8 Hz, 1H), 7.86 (d, J=0.8 Hz, 1H), 7.82 (d, J=2.5 Hz, 1H), 7.80 (s, 1H), 7.22-7.10 (m, 1H), 6.75 (dd, J=2.4, 0.9 Hz, 1H), 4.78-4.63 (m, 1H), 3.96 (s, 5H), 3.90-3.83 (m, 2H), 2.61-2.52 (m, 2H), 2.28-2.20 (m, 2H), 2.17 (s, 2H), 1.48 (s, 9H), 1.30-1.18 (m, 1H).

### Example 55. 5-(tert-butyl)-N-(2-methyl-4-(6-(1-(oxetan-3-yl)-1H-pyrazol-4-yl)pyrazolo[1,5-a]pyrazin-4-yl)benzyl)-1,2,4-oxadiazole-3-carboxamide 1. Synthesis of (2-methyl-4-(4,4,5,5-tetramethyl-1,3,2-dioxaborolan-2-yl)phenyl)methanamine dihydrochloride

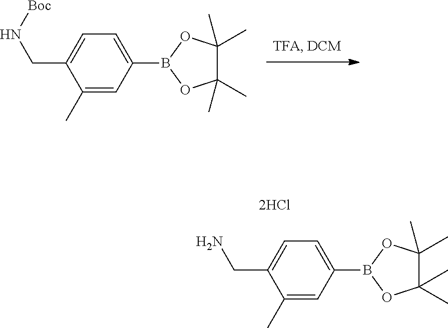 [SMILES: Cc1cc(B2OC(C)(C)C(C)(C)O2)ccc1CNC(=O)OC(C)(C)C ; Cc1cc(B2OC(C)(C)C(C)(C)O2)ccc1CN]

A solution of tert-butyl (2-methyl-4-(4,4,5,5-tetramethyl-1,3,2-dioxaborolan-2-yl)benzyl)carbamate (WO 2015089327 A1, 35.7 g, 103 mmol) in DCM, was treated with TFA (85 mL, 1110 mmol) and the reaction stirred at RT for 2 h. The mixture was concentrated in vacuo, and the residue azeotroped with toluene. The residue was suspended in sat. NaHCO₃soln., the mixture extracted with EtOAc (2×100 mL) and the combined organic extracts were washed with brine, dried over Na₂SO₄ and filtered. 4M HCl in dioxane (30 mL) was added, the solution concentrated in vacuo and the residue stirred in Et₂O (500 mL). The resulting suspension was filtered and the solid dried to afford the title compound as an off-white solid, 23.5 g, 84%. ¹H NMR (300 MHz, DMSO-d₆) δ: 8.56 (br s, 3H), 7.49-7.52 (m, 2H), 7.41 (dd, 1H), 4.01-4.04 (m, 2H), 2.34 (s, 3H), 1.27 (s, 12H).

### 2. Synthesis of 5-(tert-butyl)-N-(2-methyl-4-(4,4,5,5-tetramethyl-1,3,2-dioxaborolan-2-yl)benzyl)-1,2,4-oxadiazole-3-carboxamide

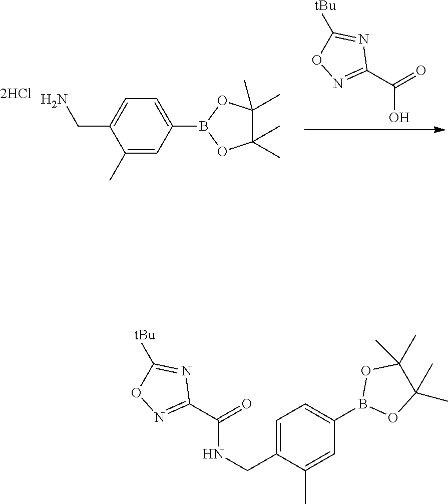 [SMILES: Cc1cc(B2OC(C)(C)C(C)(C)O2)ccc1CN ; CC(C)(C)c1nc(C(=O)O)no1 ; Cc1cc(B2OC(C)(C)C(C)(C)O2)ccc1CNC(=O)c1noc(C(C)(C)C)n1]

To a suspension of (2-methyl-4-(4,4,5,5-tetramethyl-1,3,2-dioxaborolan-2-yl)phenyl)methanamine dihydrochloride (34 g, 0.12 mol) and 5-(tert-butyl)-1,2,4-oxadiazole-3-carboxylic acid (29.2 g, 0.15 mol) in anhydrous DMF (500 mL) was added DIPEA (62 mL, 0.36 mol). The resulting mixture was cooled to 0° C., T3P® (50% in DMF, 90 mL, 0.15 mol) was added and the reaction allowed to warm to RT and stirred for 2 h. The reaction mixture was partitioned between water (300 mL) and EtOAc (300 mL) and the layers were separated. The aqueous phase was extracted with EtOAc (3×100 mL), the combined organic extracts washed with water (3×50 mL), sat. aq. solution of NaHCO₃ (50 mL) and brine (50 mL). The organic layer was dried over Na₂SO₄, filtered and concentrated in vacuo to give a beige oil. This was purified by column chromatography on silica gel eluting with heptanes/EtOAc, (100/0 to 70/30) to give the title compound as a thick pale-yellow oil which slowly solidified on standing (29.0 g, 60%).

### 3. Preparation of 5-(tert-butyl)-N-(4-(6-chloropyrazolo[1,5-a]pyrazin-4-yl)-2-methylbenzyl)-1,2,4-oxadiazole-3-carboxamide

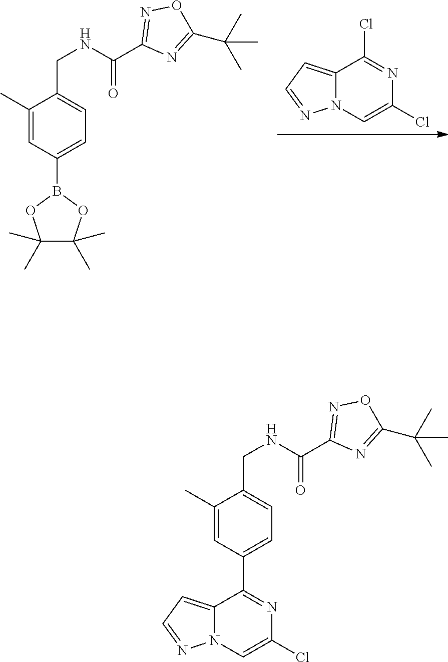 [SMILES: Cc1cc(B2OC(C)(C)C(C)(C)O2)ccc1CNC(=O)c1noc(C(C)(C)C)n1 ; Clc1cn2nccc2c(Cl)n1 ; Cc1cc(-c2nc(Cl)cn3nccc23)ccc1CNC(=O)c1noc(C(C)(C)C)n1]

A mixture of 5-(tert-butyl)-N-(2-methyl-4-(4,4,5,5-tetramethyl-1,3,2-dioxaborolan-2-yl)benzyl)-1,2,4-oxadiazole-3-carboxamide (350 mg, 876.56 μmol), 4,6-dichloropyrazolo[1,5-a]pyrazine (329.60 mg, 1.75 mmol), Pd(t-Bu₃P)₂ (22.40 mg, 43.83 μmol) and K₃PO₄ (558.20 mg, 2.63 mmol, 1.32 mL) in dioxane (4.40 mL) and water (1.10 mL) was sparged with N2 for 5 min and stirred at RT for 18 h. The mixture was diluted with EtOAc and water, the layers separated and the organic phase washed with brine. The combined aqueous layers were extracted with EtOAc, the combined organic layers were dried (MgSO₄), filtered and concentrated in vacuum. The residue was purified by column chromatography (0-100% EtOAc in Hept) to give 5-(tert-butyl)-N-(4-(6-chloropyrazolo[1,5-a]pyrazin-4-yl)-2-methylbenzyl)-1,2,4-oxadiazole-3-carboxamide (245 mg, 65.8% yield). LCMS m/z=425.0 [M+H]⁺.

### 4. Preparation of 5-(tert-butyl)-N-(2-methyl-4-(6-(1-(oxetan-3-yl)-1H-pyrazol-4-yl)pyrazolo[1,5-a]pyrazin-4-yl)benzyl)-1,2,4-oxadiazole-3-carboxamide

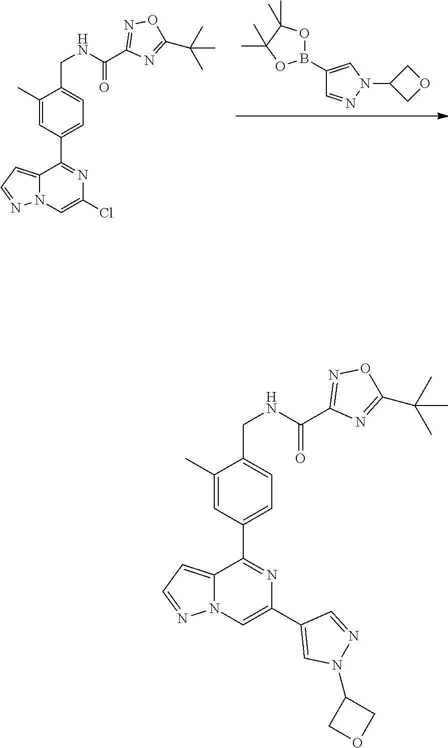

To a solution of 5-(tert-butyl)-N-(4-(6-chloropyrazolo[1,5-a]pyrazin-4-yl)-2-methylbenzyl)-1,2,4-oxadiazole-3-carboxamide (50 mg, 117.68 μmol) and 1-(oxetan-3-yl)-4-(4,4,5,5-tetramethyl-1,3,2-dioxaborolan-2-yl)pyrazole (35.32 mg, 141.22 μmol) in dioxane (2.10 mL) was added K₃PO₄ (2 M, 470.72 uL) solution in water and the resulting mixture was sparged with N2 for 5 min. tBuXPhos Pd G3 (18.70 mg, 23.54 μmol) was added and the mixture heated to 60° C. for 17 h, cooled to RT, diluted with EtOAc and washed with water and brine. The organic layer was dried (MgSO₄), filtered and concentrated in vacuum. The residue was purified by prep HPLC (Method C2, 5-60% gradient). The product was further purified by preparative TLC (97:3 DCM:MeOH) to give 5-(tert-butyl)-N-(2-methyl-4-(6-(1-(oxetan-3-yl)-1H-pyrazol-4-yl)pyrazolo[1,5-a]pyrazin-4-yl)benzyl)-1,2,4-oxadiazole-3-carboxamide (2 mg, 3.32% yield) as a white solid. LCMS m/z=513.2 [M+H]⁺ 1H NMR (500 MHz, CDCl₃) δ ppm 8.58 (s, 1H), 8.24 (s, 1H), 8.08-8.04 (m, 2H), 7.97-7.90 (m, 2H), 7.52 (d, J=7.9 Hz, 1H), 7.18 (br s, 1H), 6.97 (dd, J=2.4, 1.2 Hz, 1H), 5.60-5.51 (m, 1H), 5.17-5.10 (m, 4H), 4.78 (d, J=6.1 Hz, 2H), 2.53 (s, 3H), 1.50-1.46 (m, 9H).

### C.2 Preparation of Imidazo[1,2-c]pyrimidines

### Example 56: 1-(tert-butyl)-N-(4-(7-(1-methyl-1H-pyrazol-4-yl)imidazo[1,2-c]pyrimidin-5-yl)benzyl)-1H-pyrazole-3-carboxamide

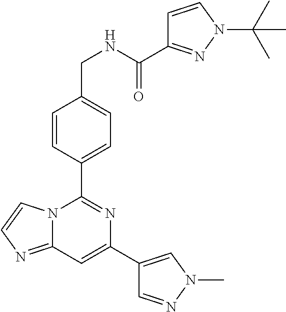 [SMILES: Cn1cc(-c2cc3nccn3c(-c3ccc(CNC(=O)c4ccn(C(C)(C)C)n4)cc3)n2)cn1]

### 1. Synthesis of 7-chloro-5-(methylthio)imidazo[1,2-c]pyrimidine

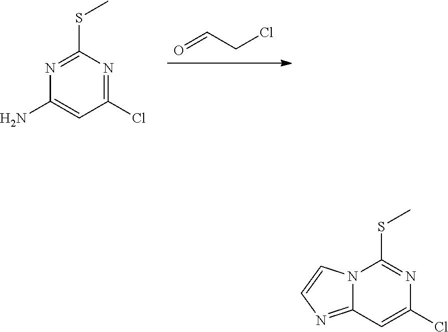

2-Chloroacetaldehyde (220 g, 0.18 L, 1.28 mol) was added to a solution of 6-chloro-2-(methylthio)pyrimidin-4-amine (150 g, 854 mmol) in dioxane (300 mL) and the reaction stirred at 100° C. for 5 h and then allowed to cool to RT overnight. The suspension was cooled to 0° C. and the solid was filtered off to give 7-chloro-5-(methylthio)imidazo[1,2-c]pyrimidine as a yellow solid (151 g, 75% yield). LCMS m/z=200.1 [M+H]⁺

### 2. Synthesis of 7-chloroimidazo[1,2-c]pyrimidin-5(6H)-one

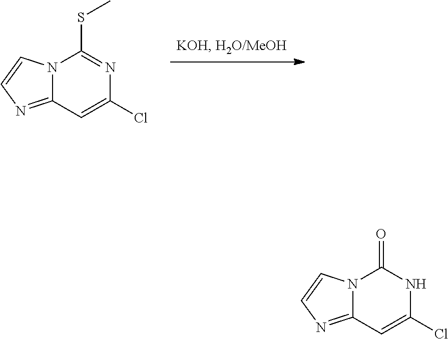

7-Chloro-5-(methylthio)imidazo[1,2-c]pyrimidine (52.2 g, 221 mmol) was suspended in MeOH (200 mL) and a solution of KOH (55.9 g, 996 mmol) in water (520 mL) was added slowly. The reaction was heated at reflux for 3 h and then was cooled to RT overnight. The mixture was acidified to pH 6 with 1M HCl and the obtained suspension was filtered. The solid was washed with MeOH then azeotroped with MeCN to provide 7-chloroimidazo[1,2-c]pyrimidin-5(6H)-one as a white solid (28.6 g, 76% yield). LCMS m/z=170.1 [M+H]⁺

### 3. Synthesis of 7-(1-methyl-1H-pyrazol-4-yl)imidazo[1,2-c]pyrimidin-5(6H)-one

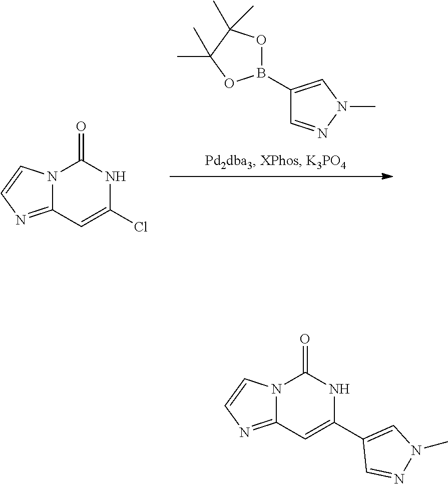

7-Chloroimidazo[1,2-c]pyrimidin-5(6H)-one (40 g, 236 mmol), 1-methyl-4-(4,4,5,5-tetramethyl-1,3,2-dioxaborolan-2-yl)-1H-pyrazole (73.6 g, 354 mmol) and XPhos (11.2 g, 24 mmol) were dissolved in IPA (1.8 L) and a 2 M solution of K₃PO₄ (150 g, 0.35 L, 708 mmol) in water was added. The mixture was purged with N2 for 15 min then Pd₂(dba)₃ (10.8 g, 12 mmol) was added, and the mixture was refluxed overnight. Additional Pd₂(dba)₃ (5 g) and 1-methyl-4-(4,4,5,5-tetramethyl-1,3,2-dioxaborolan-2-yl)-1H-pyrazole (25 g) were added and the mixture refluxed for a further 24 h. The reaction mixture was filtered to remove palladium-residues, the organic solvent was evaporated, and the residue was partitioned between water and a 1:1-mixture of heptanes: EtOAc. A white solid precipitated in both the organic and aqueous layer and the solid was filtered off. The solid was washed with water, EtOAc and MeCN and dried in vacuo to give the product (32.8 g). The layers of the filtrate were separated and the aqueous layer was cooled in an ice bath. The solution was treated with concentrated HCl to pH 6 with stirring and the resulting fine precipitate was collected, washed with H₂O and Et₂O, and dried under vacuum to give further product (9.0 g). A total of 41.8 g of 7-(1-methyl-1H-pyrazol-4-yl)imidazo[1,2-c]pyrimidin-5(6H)-one was obtained as a yellow solid (82% yield). LCMS m/z=215.0 [M+H]⁺

### 4. Synthesis of 5-chloro-7-(1-methyl-1H-pyrazol-4-yl)imidazo[1,2-c]pyrimidine

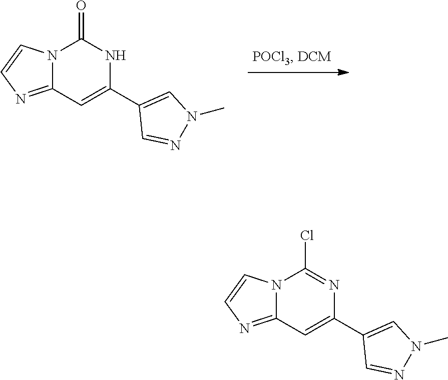 [SMILES: Cn1cc(-c2cc3nccn3c(=O)[nH]2)cn1 ; Cn1cc(-c2cc3nccn3c(Cl)n2)cn1]

POCl₃ (89.3 g, 54.1 mL, 583 mmol) was added dropwise over 5 min to an ice cooled solution of 7-(1-methyl-1H-pyrazol-4-yl)imidazo[1,2-c]pyrimidin-5(6H)-one (41.8 g, 194 mmol) and DIPEA (126 g, 971 mmol) in dry DCM (300 mL) and the reaction was allowed to warm to RT. The mixture was diluted with DCM (150 mL), then stirred for 24 h at RT. The suspension was diluted with hexanes and the solid was collected by filtration (66.0 g). The collected solid was suspended in DCM: DIPEA (5:1, 500 mL), stirred for 30 min and then a saturated aqueous solution of NaHCO₃ was added and the mixture stirred for 1 h. The mixture was filtered through Celite®, the layers were separated, and the aqueous layer extracted with DCM (3×). The organic layers were dried over Na₂SO₄ and concentrated in vacuo to provide 5-chloro-7-(1-methyl-1H-pyrazol-4-yl)imidazo[1,2-c]pyrimidine as a yellow solid (22.5 g, 50% yield). LCMS m/z=234.0 [M+H]⁺.

### 5. Synthesis of tert-butyl (4-(7-(1-methyl-1H-pyrazol-4-yl)imidazo[1,2-c]pyrimidin-5-yl)benzyl)carbamate

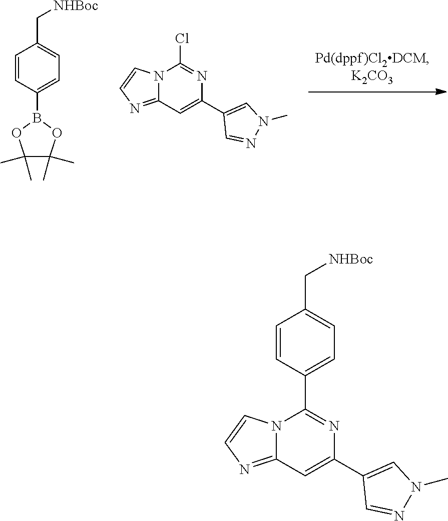

tert-Butyl (4-(7-(1-methyl-1H-pyrazol-4-yl)imidazo[1,2-c]pyrimidin-5-yl)benzyl)carbamate was obtained as a white solid, 380 mg, 63% yield, from 5-chloro-7-(1-methyl-1H-pyrazol-4-yl)imidazo[1,2-c]pyrimidine and tert-butyl (4-(4,4,5,5-tetramethyl-1,3,2-dioxaborolan-2-yl)benzyl)carbamate following a similar procedure to that described in Example 1, Step 6. LCMS m/z=405.1 [M+H]⁺

### 6. Synthesis of (4-(7-(1-methyl-1H-pyrazol-4-yl)imidazo[1,2-c]pyrimidin-5-yl)phenyl)methanamine dihydrochloride

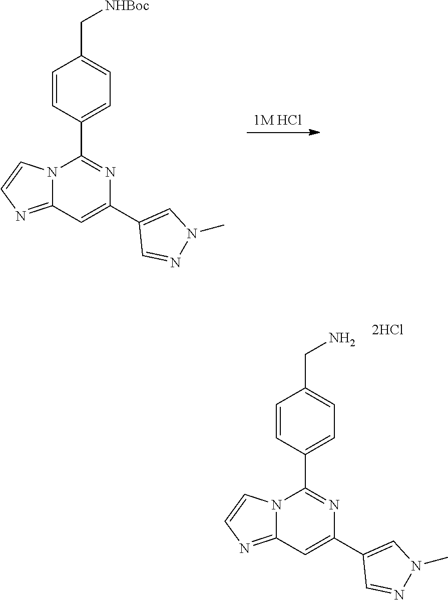

(4-(7-(1-Methyl-1H-pyrazol-4-yl)imidazo[1,2-c]pyrimidin-5-yl)phenyl)methanamine dihydrochloride was obtained, crude, 374 mg, from tert-butyl (4-(7-(1-methyl-1H-pyrazol-4-yl)imidazo[1,2-c]pyrimidin-5-yl)benzyl)carbamate following a similar procedure to that described in Example 1, step 7. LCMS m/z=305.0 [M+H]⁺

### 7. Synthesis of 1-(tert-butyl)-N-(4-(7-(1-methyl-1H-pyrazol-4-yl)imidazo[1,2-c]pyrimidin-5-yl)benzyl)-1H-pyrazole-3-carboxamide

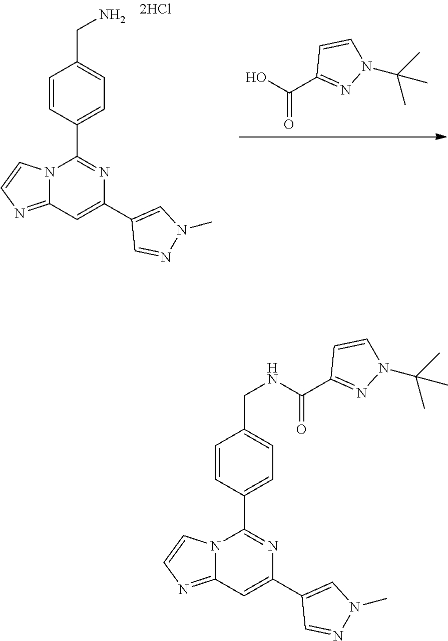 [SMILES: Cn1cc(-c2cc3nccn3c(-c3ccc(CN)cc3)n2)cn1 ; CC(C)(C)n1ccc(C(=O)O)n1 ; Cn1cc(-c2cc3nccn3c(-c3ccc(CNC(=O)c4ccn(C(C)(C)C)n4)cc3)n2)cn1]

1-(tert-Butyl)-N-(4-(7-(1-methyl-1H-pyrazol-4-yl)imidazo[1,2-c]pyrimidin-5-yl)benzyl)-1H-pyrazole-3-carboxamide was obtained, (26.2 mg, 58% yield) from (4-(7-(1-methyl-1H-pyrazol-4-yl)imidazo[1,2-c]pyrimidin-5-yl)phenyl)methanamine dihydrochloride and 1-(tert-butyl)-1H-pyrazole-3-carboxylic acid, following a similar procedure to that described in Example 1, Step 8. The crude material was purified by reverse phase HPLC (Method A, 5-55% gradient). LCMS m/z=455.2 [M+H]⁺. ¹H NMR (500 MHz, DMSO-d₆) δ: 8.70 (t, J=6.4 Hz, 1H), 8.33 (s, 1H), 8.10 (s, 1H), 7.97-7.92 (m, 4H), 7.83 (s, 1H), 7.63 (s, 1H), 7.57 (d, J=7.9 Hz, 2H), 6.67-6.65 (m, 1H), 4.56 (d, J=6.1 Hz, 2H), 3.88 (s, 3H), 1.57 (s, 9H).

### Examples 57 to 61

The compounds in the following table were prepared from (4-(7-(1-methyl-1H-pyrazol-4-yl)imidazo[1,2-c]pyrimidin-5-yl)phenyl)methanamine dihydrochloride (Example 56, step 6) and the appropriate carboxylic acid, following a similar method to that used for Example 1, step 8.

| Example | |
|---|---|
| No | Name/Structure/Carboxylic acid/Data |
| 57 | 1-(tert-butyl)-N-(4-(7-(1-methyl-1H-pyrazol-4-yl)imidazo[1,2-c]pyrimidin-5- yl)benzyl)-1H-pyrazole-4-carboxamide 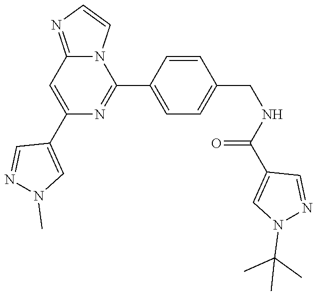 [SMILES: Cn1cc(-c2cc3nccn3c(-c3ccc(CNC(=O)c4cnn(C(C)(C)C)c4)cc3)n2)cn1] Carboxylic acid: 1-(tert-butyl)-1H-pyrazole-4-carboxylic acid 26.7 mg, 59% yield, Prep-HPLC (Method A, 5-55% gradient) LCMS m/z = 455.2 [M + H]⁺. ¹H NMR (500 MHz, DMSO-d₆) δ: 8.71 (t, J = 6.2 Hz, 1H), 8.33 (d, J = 1.8 Hz, 2H), 8.10 (s, 1H), 7.98-7.92 (m, 4H), 7.83 (s, 1H), 7.64 (d, J = 1.2 Hz, 1H), 7.55 (d, J = 7.9 Hz, 2H), 4.55 (d, J = 6.1 Hz, 2H), 3.94-3.83 (m, 3H), 1.53 (s, 9H). |
| 58 | 2-(tert-butyl)-N-(4-(7-(1-methyl-1H-pyrazol-4-yl)imidazo[1,2-c]pyrimidin-5- yl)benzyl)-2H-tetrazole-5-carboxamide 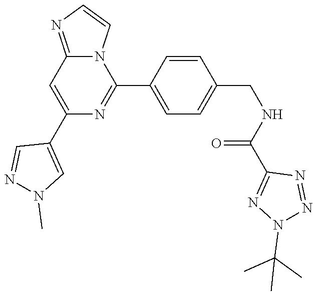 [SMILES: Cn1cc(-c2cc3nccn3c(-c3ccc(CNC(=O)c4nnn(C(C)(C)C)n4)cc3)n2)cn1] Carboxylic acid: 2-(tert-butyl)-2H-tetrazole-5-carboxylic acid 13 mg, 29% yield, Prep-HPLC (Method A, 5-50% gradient) LCMS m/z = 457.2 [M + H]⁺. ¹H NMR (500 MHz, DMSO-d₆) δ: 9.70 (t, J = 6.4 Hz, 1H), 8.35 (s, 1H), 8.11 (s, 1H), 7.98-7.95 (m, 3H), 7.85 (s, 1H), 7.67 (s, 1H), 7.59 (d, J = 8.5 Hz, 2H), 4.62 (d, J = 6.1 Hz, 2H), 3.97-3.84 (m, 3H), 1.74 (s, 9H). |
| 59 | 1-(tert-butyl)-N-(4-(7-(1-methyl-1H-pyrazol-4-yl)imidazo[1,2-c]pyrimidin-5- yl)benzyl)-1H-1,2,3-triazole-4-carboxamide 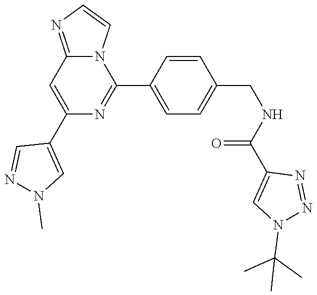 [SMILES: Cn1cc(-c2cc3nccn3c(-c3ccc(CNC(=O)c4cn(C(C)(C)C)nn4)cc3)n2)cn1] Carboxylic acid: 1-(tert-butyl)-1H-1,2,3-triazole-4-carboxylic acid 26.7 mg, 59% yield, Prep-HPLC (Method A, 5-50% gradient) LCMS m/z = 456.2 [M + H]⁺. ¹H NMR (500 MHz, DMSO-d₆) δ: 9.22 (t, J = 6.4 Hz, 1H), 8.71 (s, 1H), 8.36 (s, 1H), 8.12 (s, 1H), 7.97-7.94 (m, 3H), 7.85 (s, 1H), 7.68 (s, 1H), 7.57 (d, J = 7.9 Hz, 2H), 4.59 (d, J = 6.1 Hz, 2H), 4.05-3.79 (m, 3H), 1.64 (s, 9H). |
| 60 | N-(4-(7-(1-methyl-1H-pyrazol-4-yl)imidazo[1,2-c]pyrimidin-5-yl)benzyl)-5- (1-methylcyclopropyl)-1,2,4-oxadiazole-3-carboxamide 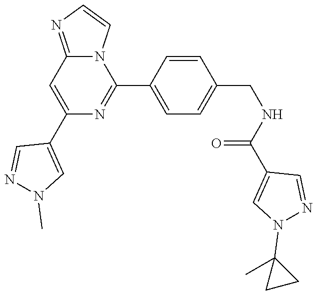 [SMILES: Cn1cc(-c2cc3nccn3c(-c3ccc(CNC(=O)c4cnn(C5(C)CC5)c4)cc3)n2)cn1] Carboxylic acid: 5-(1-methylcyclopropyl)-1,2,4-oxadiazole-3-carboxylic acid 23.1 mg, 51% yield, Prep-HPLC (Method A, 5-50% gradient) LCMS m/z = 455.1 [M + H]⁺. ¹H NMR (500 MHz, DMSO-d₆) δ: 9.57 (t, J = 6.2 Hz, 1H), 8.33 (s, 1H), 8.10 (s, 1H), 7.99-7.92 (m, 3H), 7.84 (s, 1H), 7.63 (s, 1H), 7.56 (d, J = 7.9 Hz, 2H), 4.57 (d, J = 6.7 Hz, 2H), 3.89 (s, 3H), 1.55 (s, 3H), 1.41-1.37 (m, 2H), 1.19-1.15 (m, 2H). |
| 61 | 5-(tert-butyl)-N-(4-(7-(1-methyl-1H-pyrazol-4-yl)imidazo[1,2-c]pyrimidin-5- yl)benzyl)-1,3,4-oxadiazole-2-carboxamide 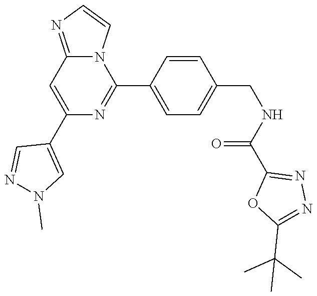 [SMILES: Cn1cc(-c2cc3nccn3c(-c3ccc(CNC(=O)c4nnc(C(C)(C)C)o4)cc3)n2)cn1] Carboxylic acid: 5-(tert-butyl)-1,3,4-oxadiazole-2-carboxylic acid 20.8 mg, 46% yield, Prep-HPLC (Method A, 5-50% gradient) LCMS m/z = 457.1 [M + H]⁺. ¹H NMR (500 MHz, DMSO-d₆) δ: 9.94 (s, 1H), 9.94 (d, J = 12.0 Hz, 1H), 8.33 (s, 1H), 8.10 (s, 1H), 7.99-7.96 (m, 2H), 7.93 (s, 1H), 7.84 (s, 1H), 7.64 (d, J = 1.2 Hz, 1H), 7.59 (d, J = 7.9 Hz, 2H), 4.59 (d, J = 6.7 Hz, 2H), 3.89 (s, 3H), 1.40 (s, 9H). |

### Example 62: 5-(2,3-cis-dimethylcyclopropyl)-N-(4-(7-(1-methyl-1H-pyrazol-4-yl)imidazo[1,2-c]pyrimidin-5-yl)benzyl)-1,2,4-oxadiazole-3-carboxamide

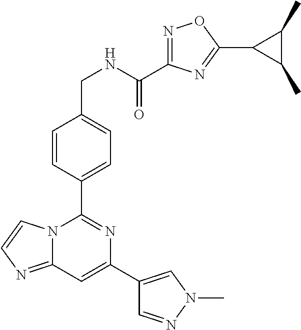 [SMILES: C[C@@H]1C(c2nc(C(=O)NCc3ccc(-c4nc(-c5cnn(C)c5)cc5nccn45)cc3)no2)[C@@H]1C]

cis racemic

### 1. Synthesis of ethyl 2,3-cis-dimethylcyclopropane-1-carboxylate

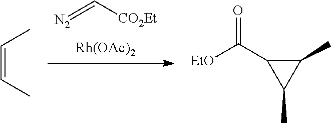

To a solution of (Z)-but-2-ene (22.5 g, 401 mmol) and Rh(OAc)₂ dimer (2.35 g, 5.3 mmol) in DCM (1.5 L) at 0° C. was added ethyl 2-diazoacetate (130 g, 1140 mmol) over 4 h. The mixture was stirred at 20° C. for 16 h. The reaction mixture was filtered through a pad of silica gel and concentrated in vacuo to give ethyl 2,3-cis-dimethylcyclopropane-1-carboxylate as a yellow oil (60 g, crude), which was carried forward without further purification.

### 2. Synthesis of 2,3-cis-dimethylcyclopropane-1-carboxylic acid

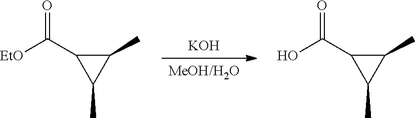

A solution of ethyl 2,3-cis-dimethylcyclopropane-1-carboxylate (60 g, 422 mmol) in MeOH (400 mL) was added to KOH (90 g, 1.6 mol) in H₂O (200 mL) at RT. After the reaction was completed, the solvent was removed by distillation in vacuo and DCM (700 mL) was added. The aqueous layer was separated and acidified with 4 M aqueous HCl solution until pH=1. The acidic aqueous phase was extracted with EtOAc (3×20 mL). The combined organic extracts were washed with brine (50 mL), dried (Na₂SO₄), filtered, and concentrated in vacuo to give 2,3-cis-dimethylcyclopropane-1-carboxylic acid (36.1 g, yield: 75% over 2 steps), which was carried forward without further purification. 3. Synthesis of ethyl 5-(2,3-cis-dimethylcyclopropyl)-1,2,4-oxadiazole-3-carboxylate

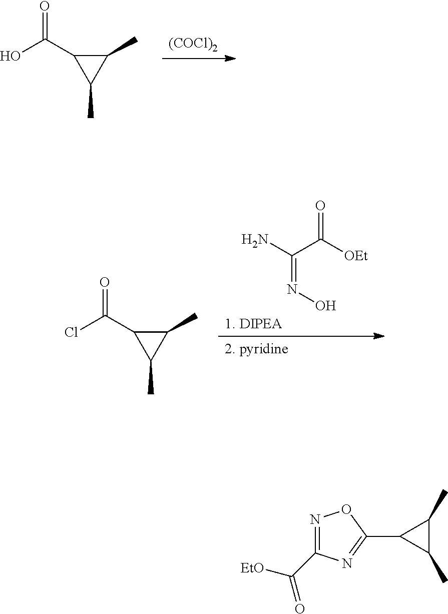

To a solution of oxalyl chloride (28.2 mL, 328 mmol) in DCM (500 mL) was added 2,3-cis-dimethylcyclopropane-1-carboxylic acid (30 g, 263 mmol), and the reaction mixture was stirred at ambient temperature for 1 h. Excess oxalyl chloride was removed by distillation to give crude 2,3-cis-dimethylcyclopropane-1-carbonyl chloride. To a solution of ethyl (E)-2-amino-2-(hydroxyimino)acetate (34.7 g, 263 mmol) and DIPEA (67.8 g, 526 mmol) in DCM (500 mL) at −15° C. was added dropwise, crude 2,3-cis-dimethylcyclopropane-1-carbonyl chloride. The resulting solution was stirred as it warmed to ambient temperature and continued to stir for 16 h. Water (800 mL) was added, and the layers were separated. The aqueous phase was extracted with DCM (300 mL×3) and the combined organic phases were dried (Na₂SO₄), filtered, and concentrated in vacuo. To the residue was added pyridine (400 mL) and the mixture heated to 110° C. and stirred for 16 h. The reaction mixture was cooled to ambient temperature and concentrated in vacuo. The crude product was purified by silica-gel column chromatography (EtOAc/hexanes, from 1% to 10%) to give ethyl 5-(2,3-cis-dimethylcyclopropyl)-1,2,4-oxadiazole-3-carboxylate as a yellow oil (9 g, yield: 16%).

### 4. Synthesis of potassium 5-(2,3-cis-dimethylcyclopropyl)-1,2,4-oxadiazole-3-carboxylate

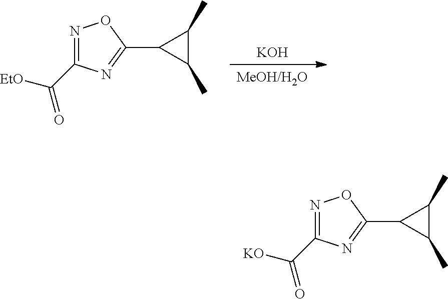

A solution of ethyl 5-(2,3-cis-dimethylcyclopropyl)-1,2,4-oxadiazole-3-carboxylate (6 g, 28.5 mmol) in MeOH (40 mL) was added to a solution of KOH (1.75 g, 31.2 mmol) dissolved in H₂O (20 mL). When the reaction was complete, the solvent was removed by distillation in vacuo and DCM (70 mL) was added. The aqueous layer was separated and concentrated in vacuo to give potassium 5-(2,3-cis-dimethylcyclopropyl)-1,2,4-oxadiazole-3-carboxylate as a white solid (5.21 g, 82% yield). LCMS m/z=183.0 [M+H]⁺

### 5. Synthesis of 5-(−2,3-cis-dimethylcyclopropyl)-N-(4-(7-(1-methyl-1H-pyrazol-4-yl)imidazo[1,2-c]pyrimidin-5-yl)benzyl)-1,2,4-oxadiazole-3-carboxamide

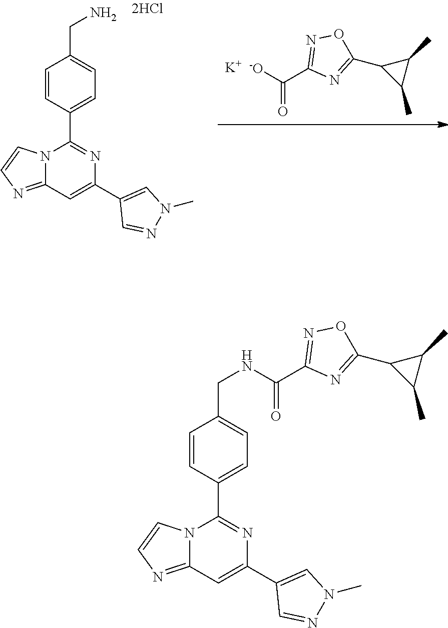

5-(−2,3-cis-Dimethylcyclopropyl)-N-(4-(7-(1-methyl-1H-pyrazol-4-yl)imidazo[1,2-c]pyrimidin-5-yl)benzyl)-1,2,4-oxadiazole-3-carboxamide was obtained (13.1 mg, 28% yield) from potassium 5-(2,3-cis-dimethylcyclopropyl)-1,2,4-oxadiazole-3-carboxylate and (4-(7-(1-methyl-1H-pyrazol-4-yl)imidazo[1,2-c]pyrimidin-5-yl)phenyl)methanamine dihydrochloride (Example 56, step 6) using a similar method to that used for Example 1, Step 8. LCMS m/z=469.1 [M+H]⁺. ¹H NMR (500 MHz, DMSO-d₆) δ: 9.70-9.52 (m, 1H), 8.34 (s, 1H), 8.19-8.07 (m, 1H), 8.05-7.92 (m, 3H), 7.89-7.81 (m, 1H), 7.64 (br d, J=1.2 Hz, 1H), 7.62-7.50 (m, 2H), 4.67-4.45 (m, 2H), 3.96-3.84 (m, 3H), 1.90 (t, J=4.6 Hz, 1H), 1.80-1.63 (m, 2H), 1.23-1.13 (m, 6H).

### Example 63: 3-(1-(fluoromethyl)cyclopropyl)-N-(4-(7-(1-methyl-1H-pyrazol-4-yl)imidazo[1,2-c]pyrimidin-5-yl)benzyl)-1,2,4-oxadiazole-5-carboxamide

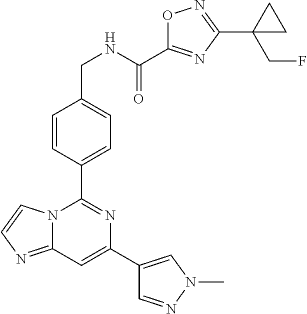 [SMILES: Cn1cc(-c2cc3nccn3c(-c3ccc(CNC(=O)c4nc(C5(CF)CC5)no4)cc3)n2)cn1]

### 1. Synthesis of (E)-1-(fluoromethyl)-N′-hydroxycyclopropane-1-carboximidamide

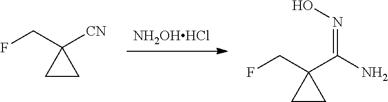 [SMILES: N#CC1(CF)CC1 ; "NH2OH·HCl" ; N/C(=N/O)C1(CF)CC1]

To a solution of 1-(fluoromethyl)cyclopropane-1-carbonitrile (850 mg, 8.5 mmol) in EtOH (15 mL) and H₂O (1 mL) were added Na₂CO₃ (1.8 g, 17 mmol) and hydroxylamine hydrochloride (1.2 g, 17 mmol) and the reaction was heated at 80° C. and stirred at that temperature for 4 h. The reaction mixture was cooled to ambient temperature, poured into H₂O (50 mL) and extracted with EtOAc (150 mL). The organic phase was dried (Na₂SO₄), filtered, and concentrated in vacuo. The crude product was purified by silica-gel column chromatography to give (E)-1-(fluoromethyl)-N′-hydroxycyclopropane-1-carboximidamide as a white solid (1 g, 90% yield), which was carried forward without further purification.

### 2. Synthesis of ethyl 3-(1-(fluoromethyl)cyclopropyl)-1,2,4-oxadiazole-5-carboxylate

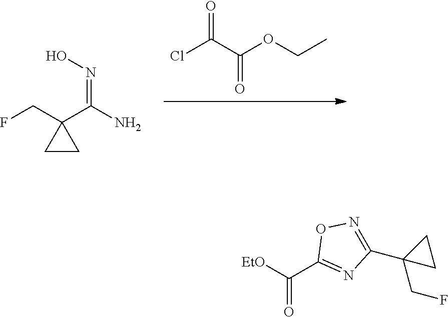

A solution of (E)-1-(fluoromethyl)-N′-hydroxycyclopropane-1-carboximidamide (5.2 g, 39 mmol) in pyridine (20 mL) was cooled to 0° C. Ethyl chlorooxoacetate (5.3 mL, 47 mmol) was added dropwise and after the addition was complete, the reaction mixture was heated at 80° C. and stirred at that temperature for 2 h. The reaction mixture was poured into ice-water (100 mL) and the aqueous phase was extracted with DCM (30 mL×3). The organic phase was washed with an HCl solution (30 mL, 1 M), followed by brine (30 mL). The organic phase was dried (Na₂SO₄), filtered and concentrated in vacuo to give ethyl 3-(1-(fluoromethyl)cyclopropyl)-1,2,4-oxadiazole-5-carboxylate (6.1 g, 85% yield), which was carried forward without further purification.

### 3. Synthesis of potassium 3-(1-(fluoromethyl)cyclopropyl)-1,2,4-oxadiazole-5-carboxylate

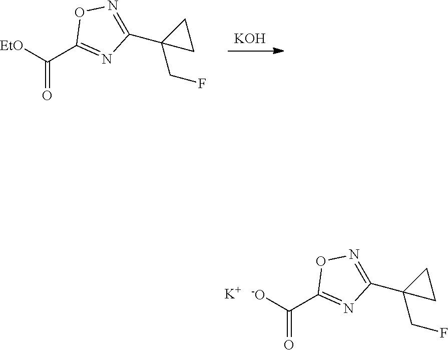

Potassium 3-(1-(fluoromethyl)cyclopropyl)-1,2,4-oxadiazole-5-carboxylate was obtained (4 g, 78% yield over 3 steps) from ethyl 3-(1-(fluoromethyl)cyclopropyl)-1,2,4-oxadiazole-5-carboxylate, following the method described in Example 64, Step 4. LCMS m/z=187.0 [M+H]⁺

### 4. Synthesis of 3-(1-(fluoromethyl)cyclopropyl)-N-(4-(7-(1-methyl-1H-pyrazol-4-yl)imidazo[1,2-c]pyrimidin-5-yl)benzyl)-1,2,4-oxadiazole-5-carboxamide

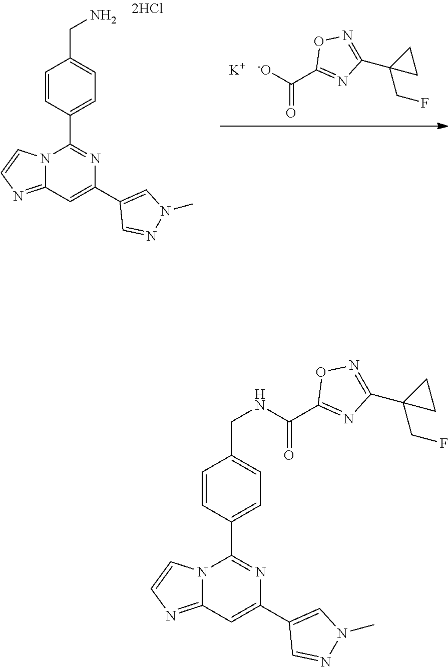

3-(1-(Fluoromethyl)cyclopropyl)-N-(4-(7-(1-methyl-1H-pyrazol-4-yl)imidazo[1,2-c]pyrimidin-5-yl)benzyl)-1,2,4-oxadiazole-5-carboxamide was obtained (7.9 mg, 17% yield) from potassium 3-(1-(fluoromethyl)cyclopropyl)-1,2,4-oxadiazole-5-carboxylate and (4-(7-(1-methyl-1H-pyrazol-4-yl)imidazo[1,2-c]pyrimidin-5-yl)phenyl)methanamine dihydrochloride (Example 56, step 6), following a similar method to that used for Example 1, step 8. LCMS m/z=473.1 [M+H]⁺. ¹H NMR (500 MHz, DMSO-d₆) δ: 9.98 (t, J=6.1 Hz, 1H), 8.33 (s, 1H), 8.10 (s, 1H), 8.00-7.94 (m, 2H), 7.94-7.91 (m, 1H), 7.84 (s, 1H), 7.64 (d, J=1.2 Hz, 1H), 7.59 (d, J=8.5 Hz, 2H), 4.78 (s, 1H), 4.69 (s, 1H), 4.59 (d, J=6.7 Hz, 2H), 3.89 (s, 3H), 1.37-1.28 (m, 4H).

### Example 64: Synthesis of 3-(tert-butyl)-N-(4-(7-(1-methyl-1H-pyrazol-4-yl)imidazo[1,2-c]pyrimidin-5-yl)benzyl)-1,2,4-oxadiazole-5-carboxamide

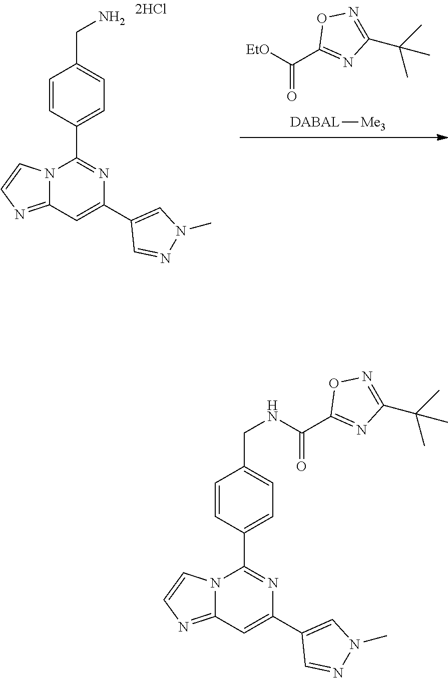

To a solution of (4-(7-(1-methyl-1H-pyrazol-4-yl)imidazo[1,2-c]pyrimidin-5-yl)phenyl)methanamine dihydrochloride (Example 56, step 6, 37 mg, 98 μmol) and ethyl 3-(tert-butyl)-1,2,4-oxadiazole-5-carboxylate (29 mg, 148 μmol) in THF (1.5 mL) was added DABAL-Me₃ (38 mg, 148 μmol) and the reaction was heated to 45° C. and was stirred at that temperature for 18 h. The reaction mixture was cooled to ambient temperature and diluted with water (1 mL), 1M HCl solution (1 mL) and EtOAc (5 mL). The layers were separated, and the aqueous phase was extracted with additional portions of EtOAc. The combined organic phase was washed brine, dried over Na₂SO₄, filtered, and concentrated. The crude material was purified by reverse phase HPLC (Method A, 5-55% gradient) to give 3-(tert-butyl)-N-(4-(7-(1-methyl-1H-pyrazol-4-yl)imidazo[1,2-c]pyrimidin-5-yl)benzyl)-1,2,4-oxadiazole-5-carboxamide (5.6 mg, yield 12%).). LCMS m/z=457.2 [M+H]⁺. ¹H NMR (500 MHz, DMSO-d₆) δ: 10.00 (br t, J=6.1 Hz, 1H), 8.35 (s, 1H), 8.12 (s, 1H), 8.02-7.95 (m, 3H), 7.86 (s, 1H), 7.69 (s, 1H), 7.60 (d, J=7.9 Hz, 2H), 4.60 (d, J=6.1 Hz, 2H), 3.89 (s, 3H), 1.41-1.34 (m, 9H).

### C.3 Preparation of Pyrazolo[1,5-a]pyridines

### Example 65: N-(2-methyl-4-(6-(1-methyl-1H-pyrazol-4-yl)pyrazolo[1,5-a]pyridin-4-yl)benzyl)-5-(1-methylcyclopropyl)-1,2,4-oxadiazole-3-carboxamide

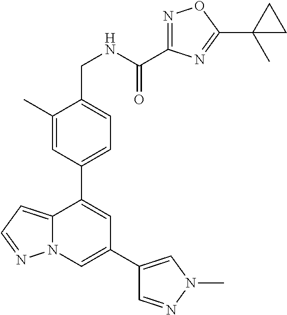 [SMILES: Cc1cc(-c2cc(-c3cnn(C)c3)cn3nccc23)ccc1CNC(=O)c1noc(C2(C)CC2)n1]

### 1. Synthesis of 6-bromopyrazolo[1,5-a]pyridin-4-ol

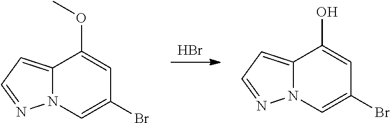

A solution of 6-bromo-4-methoxypyrazolo[1,5-a]pyridine (300 mg, 1.32 mmol) in aqueous HBr (267 mg, 1.32 mmol, 40% purity, 10 mL) was stirred at 130° C. for 12 h. The reaction mixture was cooled to ambient temperature and a solution of satd. Aq. Na₂CO₃ was added to adjust the pH to 8. The quenched reaction mixture was concentrated under vacuum to give crude material which was purified by silica gel column chromatography (PE/EtOAc=7:3) to give 6-bromopyrazolo[1,5-a]pyridin-4-ol as a brown solid (150 mg, 51% yield). LCMS m/z=212.9, 214.9 [M+H]⁺.

### 2. Synthesis of 6-bromopyrazolo[1,5-a]pyridin-4-yl trifluoromethanesulfonate

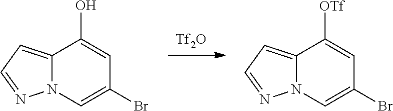 [SMILES: "Tf2O" ; Oc1cc(Br)cn2nccc12 ; O=S(=O)(Oc1cc(Br)cn2nccc12)C(F)(F)F]

To a solution of 6-bromopyrazolo[1,5-a]pyridin-4-ol (100 mg, 0.47 mmol) in DCM (10 mL) was added TEA (143 mg, 1.41 mmol) and the reaction mixture was cooled to 0° C. in an ice-water cooling bath. Triflic anhydride (265 mg, 0.94 mmol) was added and the reaction stirred at 0° C. for 30 mins. The reaction mixture was concentrated and the crude material was purified by prep-TLC (PE/EtOAc=10:1) to give 6-bromopyrazolo[1,5-a]pyridin-4-yl trifluoromethanesulfonate as a clear oil (150 mg, 84% yield). LCMS m/z=344.9, 346.9 [M+H]⁺.

### 3. Synthesis of tert-butyl (4-(6-bromopyrazolo[1,5-a]pyridin-4-yl)-2-methylbenzyl)carbamate

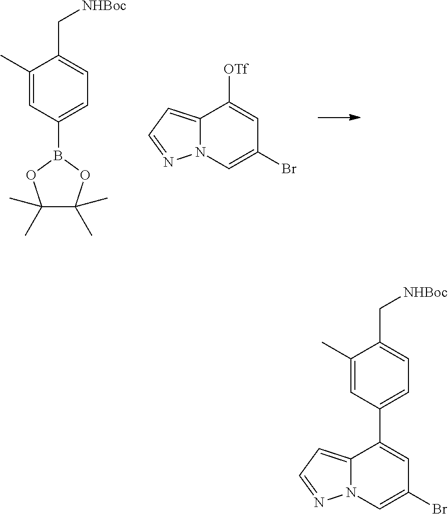

A mixture of tert-butyl (2-methyl-4-(4,4,5,5-tetramethyl-1,3,2-dioxaborolan-2-yl)benzyl)carbamate (208 mg, 0.60 mmol), 6-bromopyrazolo[1,5-a]pyridin-4-yl trifluoromethanesulfonate (207 mg, 0.60 mmol), K₂CO₃ (249 mg, 1.80 mmol), and Pd(dppf)Cl₂•DCM (49 mg, 60 μmol) were dissolved in dioxane (4.8 mL) and water (1.2 mL) and N2 was bubbled through the mixture for 5 mins. The reaction mixture was heated to 100° C. under an atmosphere of N2 for 1 h. The reaction was cooled to ambient temperature and filtered through Celite®. The filtrate was concentrated and the crude material was purified by silica gel column chromatography (EtOAc/heptanes, from 2% to 100%) to give tert-butyl (4-(6-bromopyrazolo[1,5-a]pyridin-4-yl)-2-methylbenzyl)carbamate as a clear oil (160 mg, 64% yield). LCMS m/z=416.0, 418.0. [M+H]⁺.

### 4. Synthesis of tert-butyl (2-methyl-4-(6-(1-methyl-1H-pyrazol-4-yl)pyrazolo[1,5-a]pyridin-4-yl)benzyl)carbamate

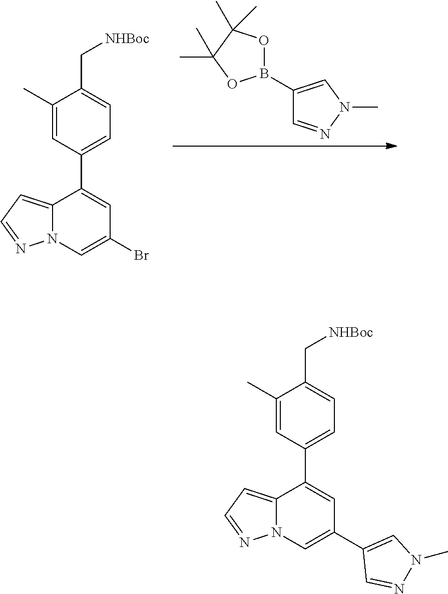

A mixture of tert-butyl (4-(6-bromopyrazolo[1,5-a]pyridin-4-yl)-2-methylbenzyl)carbamate (75 mg, 0.18 mmol), 1-methyl-4-(4,4,5,5-tetramethyl-1,3,2-dioxaborolan-2-yl)-1H-pyrazole (56 mg, 0.27 mmol), K₂CO₃ (75 mg, 0.54 mmol), and Pd(dppf)Cl₂•DCM (15 mg, 18 μmol) were dissolved in dioxane (1.4 mL) and water (0.4 mL) and N2 was bubbled through the mixture for 5 mins. The reaction mixture was heated to 100° C. under an atmosphere of N2 and stirred for 1 h. The reaction mixture was cooled to ambient temperature and filtered through Celite®. The filtrate was concentrated and the crude material was purified by silica gel column chromatography (EtOAc/heptanes, from 2% to 100%) to give tert-butyl (2-methyl-4-(6-(1-methyl-1H-pyrazol-4-yl)pyrazolo[1,5-a]pyridin-4-yl)benzyl)carbamate as a clear oil (44 mg, 59% yield). LCMS m/z=418.1 [M+H]⁺

### 5. Synthesis of (2-methyl-4-(6-(1-methyl-1H-pyrazol-4-yl)pyrazolo[1,5-a]pyridin-4-yl)phenyl)methanamine dihydrochloride

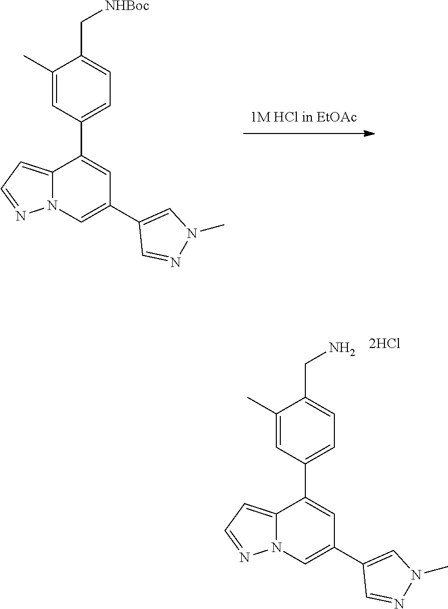 [SMILES: Cc1cc(-c2cc(-c3cnn(C)c3)cn3nccc23)ccc1CNC(=O)OC(C)(C)C ; Cc1cc(-c2cc(-c3cnn(C)c3)cn3nccc23)ccc1CN]

To a solution of tert-butyl (2-methyl-4-(6-(1-methyl-1H-pyrazol-4-yl)pyrazolo[1,5-a]pyridin-4-yl)benzyl)carbamate (44 mg, 0.11 mmol) in minimal EtOAc was added an HCl solution (1M in EtOAc, 1 mL) and the reaction stirred at ambient temperature for 3 days. The reaction mixture was concentrated to give (2-methyl-4-(6-(1-methyl-1H-pyrazol-4-yl)pyrazolo[1,5-a]pyridin-4-yl)phenyl)methanamine dihydrochloride (40 mg, crude), which was carried forward without further purification. LCMS m/z=318.0 [M+H]⁺

### 6. Synthesis of N-(2-methyl-4-(6-(1-methyl-1H-pyrazol-4-yl)pyrazolo[1,5-a]pyridin-4-yl)benzyl)-5-(1-methylcyclopropyl)-1,2,4-oxadiazole-3-carboxamide

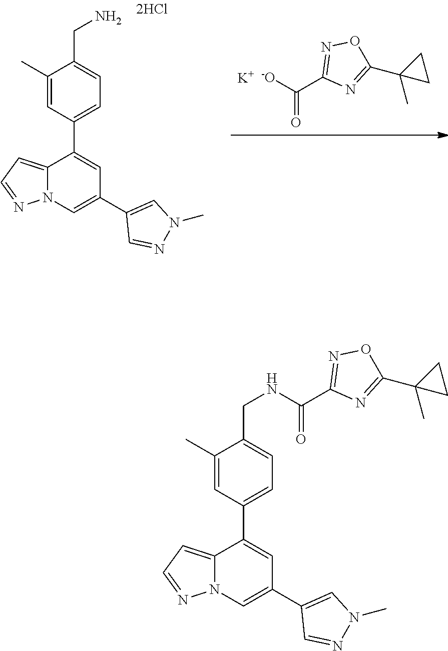

N-(2-Methyl-4-(6-(1-methyl-1H-pyrazol-4-yl)pyrazolo[1,5-a]pyridin-4-yl)benzyl)-5-(1-methylcyclopropyl)-1,2,4-oxadiazole-3-carboxamide was obtained as a white solid (11.3 mg, 47% yield) from (2-methyl-4-(6-(1-methyl-1H-pyrazol-4-yl)pyrazolo[1,5-a]pyridin-4-yl)phenyl)methanamine dihydrochloride and potassium 5-(1-methylcyclopropyl)-1,2,4-oxadiazole-3-carboxylate following a similar method to that used in Example 5. The crude product was purified by prep-HPLC (Method C1, 5-65% gradient). LCMS m/z=468.2 [M+H]⁺. ¹H NMR (500 MHz, DMSO-d₆) δ: 9.42 (t, J=6.1 Hz, 1H), 8.99 (s, 1H), 8.32 (s, 1H), 8.06 (s, 1H), 8.01 (d, J=2.4 Hz, 1H), 7.61-7.50 (m, 3H), 7.39 (d, J=7.9 Hz, 1H), 6.67 (dd, J=2.4 Hz, 1.2 Hz, 1H), 4.51 (d, J=6.1 Hz, 2H), 3.88 (s, 3H), 2.43 (s, 3H), 1.55 (s, 3H), 1.42-1.36 (m, 2H), 1.20-1.12 (m, 2H).

### Example 66: Synthesis of 1-(tert-butyl)-N-(2-methyl-4-(6-(1-methyl-1H-pyrazol-4-yl)pyrazolo[1,5-a]pyridin-4-yl)benzyl)-1H-1,2,3-triazole-4-carboxamide

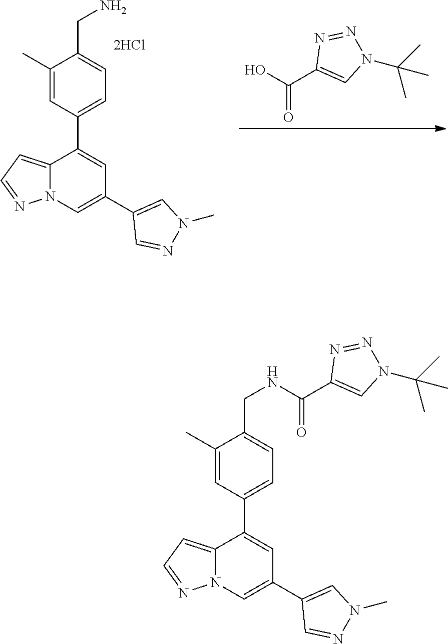 [SMILES: Cc1cc(-c2cc(-c3cnn(C)c3)cn3nccc23)ccc1CN ; CC(C)(C)n1cc(C(=O)O)nn1 ; Cc1cc(-c2cc(-c3cnn(C)c3)cn3nccc23)ccc1CNC(=O)c1cn(C(C)(C)C)nn1]

1-(tert-Butyl)-N-(2-methyl-4-(6-(1-methyl-1H-pyrazol-4-yl)pyrazolo[1,5-a]pyridin-4-yl)benzyl)-1H-1,2,3-triazole-4-carboxamide was obtained as a pink solid (10.7 mg, 45% yield) from (2-methyl-4-(6-(1-methyl-1H-pyrazol-4-yl)pyrazolo[1,5-a]pyridin-4-yl)phenyl) methanamine dihydrochloride (Example 65, step 6) and 1-(tert-butyl)-1H-1,2,3-triazole-4-carboxylic acid following a similar method to that described in Example 4. The crude material was purified by reverse phase HPLC (Method A, 5-60% gradient). LCMS m/z=469.3 [M+H]⁺. ¹H NMR (500 MHz, DMSO-d₆) δ: 9.01 (t, J=6.1 Hz, 1H), 8.99-8.97 (m, 1H), 8.71 (s, 1H), 8.32 (s, 1H), 8.05 (s, 1H), 8.01 (d, J=2.4 Hz, 1H), 7.60-7.54 (m, 2H), 7.51 (d, J=1.2 Hz, 1H), 7.39 (d, J=7.9 Hz, 1H), 6.70-6.64 (m, 1H), 4.52 (d, J=6.1 Hz, 2H), 3.88 (s, 3H), 2.44 (s, 3H), 1.64 (s, 9H).

### Example 67: N-(2-fluoro-4-(6-(1-methyl-1H-pyrazol-4-yl)pyrazolo[1,5-a]pyridin-4-yl)benzyl)-5-(1-methylcyclopropyl)-1,2,4-oxadiazole-3-carboxamide

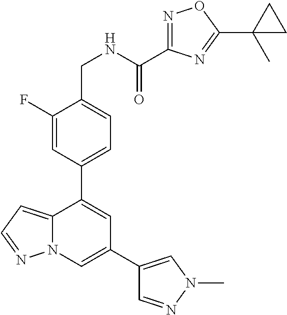 [SMILES: Cn1cc(-c2cc(-c3ccc(CNC(=O)c4noc(C5(C)CC5)n4)c(F)c3)c3ccnn3c2)cn1]

### 1. Synthesis of tert-butyl (4-(6-bromopyrazolo[1,5-a]pyridin-4-yl)-2-fluorobenzyl) carbamate

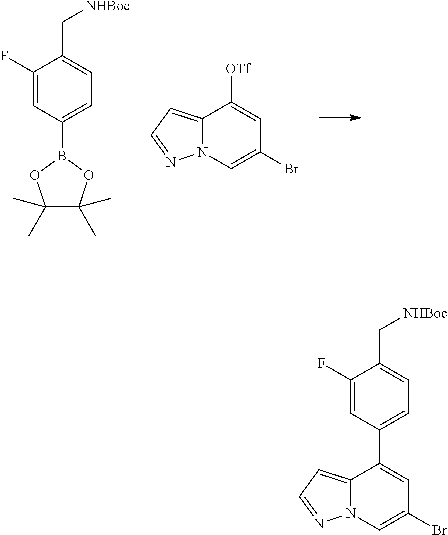 [SMILES: CC(C)(C)OC(=O)NCc1ccc(B2OC(C)(C)C(C)(C)O2)cc1F ; O=S(=O)(Oc1cc(Br)cn2nccc12)C(F)(F)F ; CC(C)(C)OC(=O)NCc1ccc(-c2cc(Br)cn3nccc23)cc1F]

A mixture of tert-butyl (2-fluoro-4-(4,4,5,5-tetramethyl-1,3,2-dioxaborolan-2-yl)benzyl)carbamate (211 mg, 0.60 mmol), 6-bromopyrazolo[1,5-a]pyridin-4-yl trifluoromethanesulfonate (207 mg, 0.60 mmol), K₂CO₃ (249 mg, 1.80 mmol), and Pd(dppf)Cl₂•DCM (49 mg, 60 μmol) were dissolved in dioxane (4.8 mL) and water (1.2 mL) and N2 was bubbled through the mixture for 5 mins. The reaction mixture was heated to 100° C. under an atmosphere of N2 and was stirred for 1 h. The reaction mixture was cooled to ambient temperature and filtered through Celite®. The filtrate was concentrated and the crude material was purified by silica gel column chromatography (EtOAc/heptanes, from 2% to 100%) to give tert-butyl (4-(6-bromopyrazolo[1,5-a]pyridin-4-yl)-2-fluorobenzyl)carbamate as a white solid (151 mg, 60% yield). LCMS m/z=419.9, 421.9 [M+H]⁺

### 2. Synthesis of tert-butyl (2-fluoro-4-(6-(1-methyl-1H-pyrazol-4-yl)pyrazolo[1,5-a]pyridin-4-yl)benzyl)carbamate

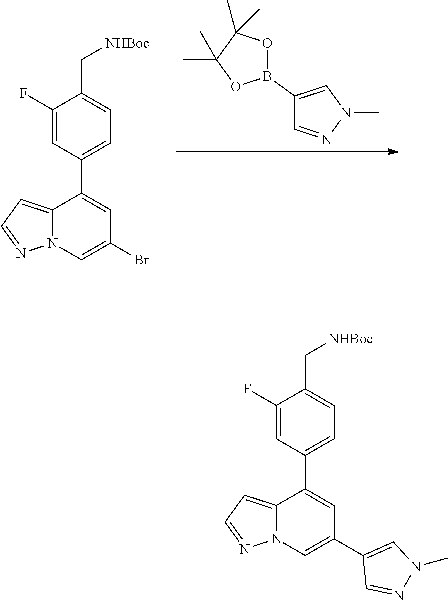

A mixture of tert-butyl (4-(6-bromopyrazolo[1,5-a]pyridin-4-yl)-2-fluorobenzyl)carbamate (151 mg, 0.36 mmol), 1-methyl-4-(4,4,5,5-tetramethyl-1,3,2-dioxaborolan-2-yl)-1H-pyrazole (112 mg, 0.54 mmol), K₂CO₃ (149 mg, 1.1 mmol), and Pd(dppf)Cl₂•DCM (29 mg, 36 μmol) were dissolved in dioxane (2.9 mL) and water (0.7 mL) and N2 was bubbled through the mixture for 5 mins. The reaction was heated to 100° C. under an atmosphere of N2 and was stirred for 1 h. The reaction mixture was cooled to ambient temperature and filtered through Celite®. The filtrate was concentrated and the crude material was purified by silica gel column chromatography (EtOAc/heptanes, from 2% to 100%) to give tert-butyl (2-fluoro-4-(6-(1-methyl-1H-pyrazol-4-yl)pyrazolo[1,5-a]pyridin-4-yl)benzyl)carbamate as an off-white solid (115 mg, 76% yield). LCMS m/z=422.1 [M+H]⁺

### 3. Synthesis of(2-fluoro-4-(6-(1-methyl-1H-pyrazol-4-yl)pyrazolo[1,5-a]pyridin-4-yl)phenyl)methanamine dihydrochloride

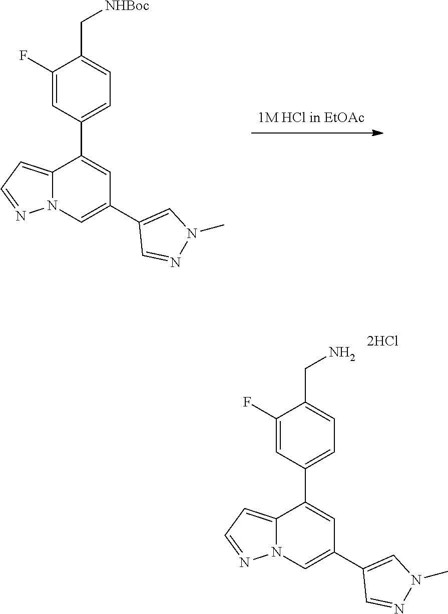 [SMILES: Cn1cc(-c2cc(-c3ccc(CNC(=O)OC(C)(C)C)c(F)c3)c3ccnn3c2)cn1 ; Cn1cc(-c2cc(-c3ccc(CN)c(F)c3)c3ccnn3c2)cn1]

To a solution of tert-butyl (2-fluoro-4-(6-(1-methyl-1H-pyrazol-4-yl)pyrazolo[1,5-a]pyridin-4-yl)benzyl)carbamate (115 mg, 0.27 mmol) in minimal EtOAc was added an HCl solution (1M in EtOAc, 2.7 mL). The reaction mixture was stirred at ambient temperature for 3 days. The reaction mixture was concentrated to give (2-fluoro-4-(6-(1-methyl-1H-pyrazol-4-yl)pyrazolo[1,5-a]pyridin-4-yl)phenyl)methanamine dihydrochloride (115 mg, crude), which was carried forward without further purification. LCMS m/z=322.0 [M+H]⁺

### 4. Synthesis of N-(2-fluoro-4-(6-(1-methyl-1H-pyrazol-4-yl)pyrazolo[1,5-a]pyridin-4-yl)benzyl)-5-(1-methylcyclopropyl)-1,2,4-oxadiazole-3-carboxamide

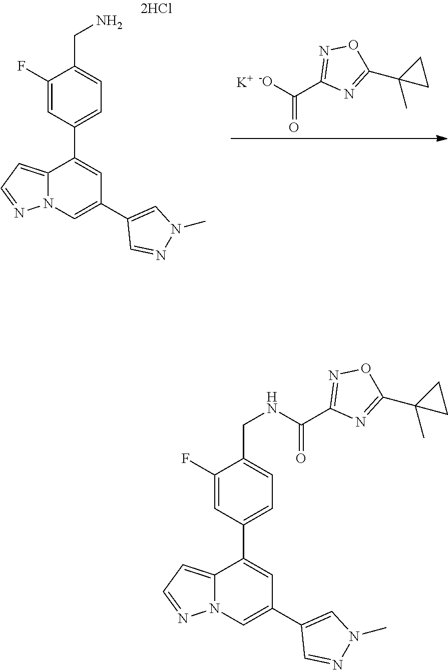

N-(2-Fluoro-4-(6-(1-methyl-1H-pyrazol-4-yl)pyrazolo[1,5-a]pyridin-4-yl)benzyl)-5-(1-methylcyclopropyl)-1,2,4-oxadiazole-3-carboxamide was obtained as a white solid (8.3 mg, 25% yield) from (2-fluoro-4-(6-(1-methyl-1H-pyrazol-4-yl)pyrazolo[1,5-a]pyridin-4-yl)phenyl)methanamine dihydrochloride and potassium 5-(1-methylcyclopropyl)-1,2,4-oxadiazole-3-carboxylate, following a similar method to that used for Example 1, step 8. The crude material was purified by prep-HPLC (Method A, 5-60% gradient). LCMS m/z=472.4 [M+H]⁺. ¹H NMR (500 MHz, DMSO-d₆) δ: 9.51 (t, J=6.1 Hz, 1H), 9.03 (s, 1H), 8.34 (s, 1H), 8.07 (s, 1H), 8.04 (d, J=2.4 Hz, 1H), 7.67-7.58 (m, 3H), 7.52 (t, J=7.9 Hz, 1H), 6.71 (d, J=2.4 Hz, 1H), 4.57 (d, J=6.1 Hz, 2H), 3.88 (s, 3H), 1.54 (s, 3H), 1.46-1.35 (m, 2H), 1.22-1.12 (m, 2H).

### Example 68: Synthesis of 5-(tert-butyl)-N-(2-fluoro-4-(6-(1-methyl-1H-pyrazol-4-yl)pyrazolo[1,5-a]pyridin-4-yl)benzyl)-1,2,4-oxadiazole-3-carboxamide

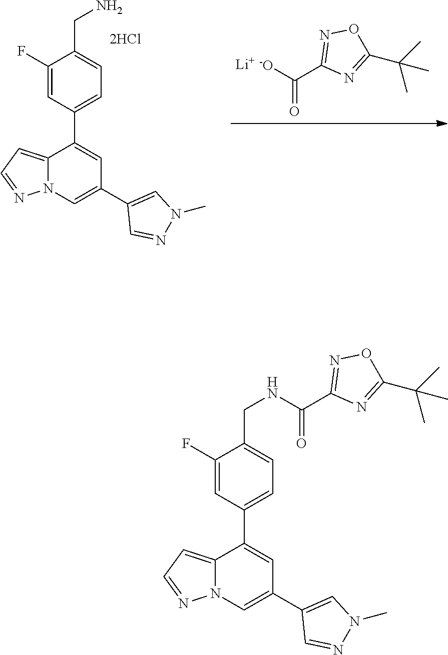

5-(tert-Butyl)-N-(2-fluoro-4-(6-(1-methyl-1H-pyrazol-4-yl)pyrazolo[1,5-a]pyridin-4-yl)benzyl)-1,2,4-oxadiazole-3-carboxamide was obtained as a white solid (7.3 mg, 22% yield) from (2-fluoro-4-(6-(1-methyl-1H-pyrazol-4-yl)pyrazolo[1,5-a]pyridin-4-yl)phenyl)methanamine dihydrochloride (Example 67, step 3) and lithium 5-(tert-butyl)-1,2,4-oxadiazole-3-carboxylate following a similar method to that used in Example 67, step 4. The crude material was purified by prep-HPLC method A, gradient 5-65%. LCMS m/z=474.4 [M+H]^{+ 1}H NMR (500 MHz, DMSO-d₆) δ: 9.56 (t, J=6.1 Hz, 1H), 9.03 (s, 1H), 8.34 (s, 1H), 8.07 (s, 1H), 8.04 (d, J=1.8 Hz, 1H), 7.70-7.60 (m, 3H), 7.53 (t, J=7.9 Hz, 1H), 6.76-6.67 (m, 1H), 4.58 (d, J=6.1 Hz, 2H), 3.88 (s, 3H), 1.43 (s, 9H).

### Example 69: Synthesis of 1-(tert-butyl)-N-(2-fluoro-4-(6-(1-methyl-1H-pyrazol-4-yl)pyrazolo[1,5-a]pyridin-4-yl)benzyl)-1H-1,2,3-triazole-4-carboxamide

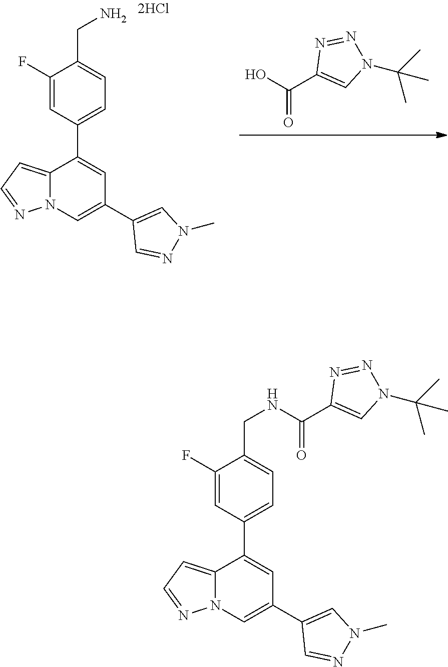 [SMILES: Cn1cc(-c2cc(-c3ccc(CN)c(F)c3)c3ccnn3c2)cn1 ; CC(C)(C)n1cc(C(=O)O)nn1 ; Cn1cc(-c2cc(-c3ccc(CNC(=O)c4cn(C(C)(C)C)nn4)c(F)c3)c3ccnn3c2)cn1]

1-(tert-Butyl)-N-(2-fluoro-4-(6-(1-methyl-1H-pyrazol-4-yl)pyrazolo[1,5-a]pyridin-4-yl)benzyl)-1H-1,2,3-triazole-4-carboxamide was obtained as a white solid (3.5 mg, 10% yield) from (2-fluoro-4-(6-(1-methyl-1H-pyrazol-4-yl)pyrazolo[1,5-a]pyridin-4-yl)phenyl) methanamine dihydrochloride (Example 67, step 3) and 1-(tert-butyl)-1H-1,2,3-triazole-4-carboxylic acid, following a similar method to that used for Example 1, step 8. The crude material was purified by prep-HPLC (Method A, 5-60% gradient). LCMS m/z=473.5 [M+H]⁺. ¹H NMR (500 MHz, DMSO-d₆) δ: 9.12 (t, J=6.1 Hz, 1H), 9.03 (s, 1H), 8.72 (s, 1H), 8.37-8.30 (m, 1H), 8.07 (s, 1H), 8.03 (d, J=2.4 Hz, 1H), 7.63-7.59 (m, 3H), 7.54-7.47 (m, 1H), 6.71 (d, J=2.4 Hz, 1H), 4.58 (d, J=6.1 Hz, 2H), 3.88 (s, 3H), 1.64 (s, 9H).

### Example 70: Synthesis of 1-(tert-butyl)-N-(2-fluoro-4-(6-(1-methyl-1H-pyrazol-4-yl)pyrazolo[1,5-a]pyridin-4-yl)benzyl)-1H-pyrazole-3-carboxamide

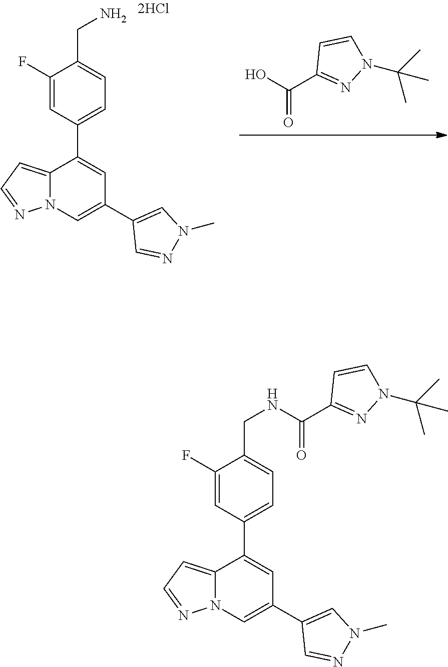 [SMILES: Cn1cc(-c2cc(-c3ccc(CN)c(F)c3)c3ccnn3c2)cn1 ; CC(C)(C)n1ccc(C(=O)O)n1 ; Cn1cc(-c2cc(-c3ccc(CNC(=O)c4ccn(C(C)(C)C)n4)c(F)c3)c3ccnn3c2)cn1]

1-(tert-Butyl)-N-(2-fluoro-4-(6-(1-methyl-1H-pyrazol-4-yl)pyrazolo[1,5-a]pyridin-4-yl)benzyl)-1H-pyrazole-3-carboxamide was obtained as a white solid (17.9 mg, 53% yield) from (2-fluoro-4-(6-(1-methyl-1H-pyrazol-4-yl)pyrazolo[1,5-a]pyridin-4-yl)phenyl)methanamine dihydrochloride (Example 67, step 3) and 1-(tert-butyl)-1H-pyrazole-3-carboxylic acid, following a similar method to that described in Example 1, step 8. LCMS m/z=472.5 [M+H]⁺. ¹H NMR (500 MHz, DMSO-d₆) δ: 9.07-8.98 (m, 1H), 8.61 (t, J=6.1 Hz, 1H), 8.33 (s, 1H), 8.07 (s, 1H), 8.03 (d, J=2.4 Hz, 1H), 7.93 (d, J=2.4 Hz, 1H), 7.68-7.57 (m, 3H), 7.56-7.47 (m, 1H), 6.75-6.70 (m, 1H), 6.66 (d, J=2.4 Hz, 1H), 4.56 (d, J=6.1 Hz, 2H), 3.88 (s, 3H), 1.57 (s, 9H).

### Example 71: 3-(1-fluoro-2-methylpropan-2-yl)-N-(2-fluoro-4-(6-(1-methyl-1H-pyrazol-4-yl)pyrazolo[1,5-a]pyridin-4-yl)benzyl)-1,2,4-oxadiazole-5-carboxamide

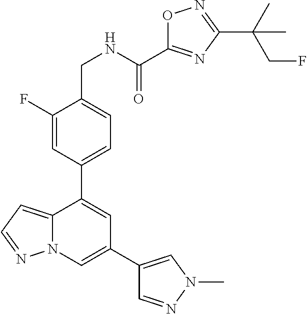 [SMILES: Cn1cc(-c2cc(-c3ccc(CNC(=O)c4nc(C(C)(C)CF)no4)c(F)c3)c3ccnn3c2)cn1]

### 1. Synthesis of 3-hydroxy-2,2-dimethylpropanenitrile

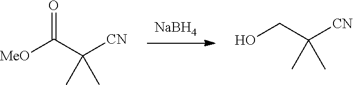

To a solution of methyl 2-cyano-2-methylpropanoate (100 g, 786 mmol) in THF (800 mL) and water (2 L), NaBH₄ (89.3 g, 2.36 mol) was added slowly over 1 h at 15° C. (ice-water bath) and the mixture was stirred at 20° C. for 4 h. The reaction was quenched by slow addition of aqueous HCl solution (6 M) to pH<7. The mixture was extracted with EtOAc (3×) and the organic layer was washed with 2N NaOH and brine. The organic phase was dried over Na₂SO₄, filtered, and concentrated under vacuum to give a colorless oil (48 g). Heptane was added and mixture concentrated again to remove the volatiles. Crude 3-hydroxy-2,2-dimethylpropanenitrile was obtained as a colorless oil (47 g, 60% yield) and was carried forward without further purification. ¹H NMR (300 MHz, CDCl₃) δ: 3.56 (s, 2H), 2.51 (br s, 1H), 1.31 (s, 6H).

### 2. Synthesis of (E)-N′,3-dihydroxy-2,2-dimethylpropanimidamide

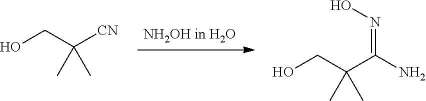

To a solution of 3-hydroxy-2,2-dimethylpropanenitrile (51.0 g, 515 mmol) in EtOH (800 mL), hydroxylamine (50% weight in water, 120 mL, 2.06 mol) was added at 25° C. and the reaction was stirred at 70° C. overnight. The mixture was cooled and concentrated under vacuum and the water was removed azeotropically with toluene (2×) and THF (1×). The white solid was triturated in Et₂O, filtered, and dried again under vacuum to give (E)-N′,3-dihydroxy-2,2-dimethylpropanimidamide as a white solid (62.5 g, 91% yield), which was carried forward without further purification. ¹H NMR (300 MHz, DMSO-d₆) δ: 8.85 (s, 1H), 5.20 (s, 2H), 4.54 (br s, 1H), 3.31 (s, 2H), 1.03 (s, 6H).

### 3. Synthesis of ethyl 3-(1-hydroxy-2-methylpropan-2-yl)-1,2,4-oxadiazole-5-carboxylate

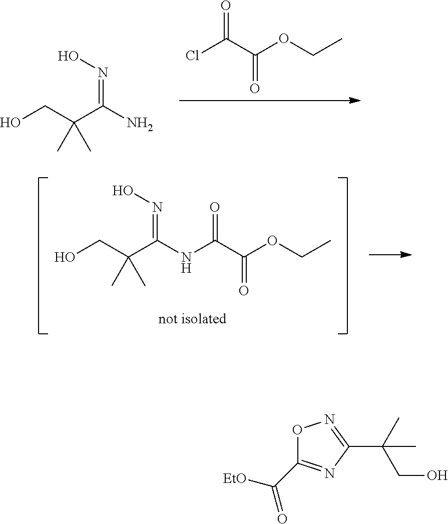

Ethyl 2-chloro-2-oxo-acetate (53.0 mL, 473 mmol) was added to a solution of (E)-N′,3-dihydroxy-2,2-dimethylpropanimidamide (62.5 g, 473 mmol) in pyridine (1 L) cooled with an ice-water bath. After 1 h, the reaction mixture was heated to 80° C. and was stirred at that temperature for 16 h. The mixture was concentrated under vacuum to remove pyridine. The residue was dissolved in EtOAc (400 mL) and water (300 mL) and extracted with EtOAc (3×300 mL). The combined organic phases were washed with water (100 mL) and brine, dried over Na₂SO₄, filtered, and concentrated under vacuum. The residue was purified by silica gel column chromatography (eluting with DCM/EtOAc, from 10/1 to 1/1) to give a mixture of ethyl 3-(1-hydroxy-2-methylpropan-2-yl)-1,2,4-oxadiazole-5-carboxylate with 20 mol % of 3-hydroxy-2,2-dimethylpropanenitrile. Most of the 3-hydroxy-2,2-dimethylpropanenitrile was removed by Kugelrohr distillation at 90° C. to afford ethyl 3-(1-hydroxy-2-methylpropan-2-yl)-1,2,4-oxadiazole-5-carboxylate as a colorless oil (22.5 g, 22% yield), which was carried forward without further purification.

### 4. Synthesis of ethyl 3-(2-methyl-1-(((trifluoromethyl)sulfonyl)oxy)propan-2-yl)-1,2,4-oxadiazole-5-carboxylate

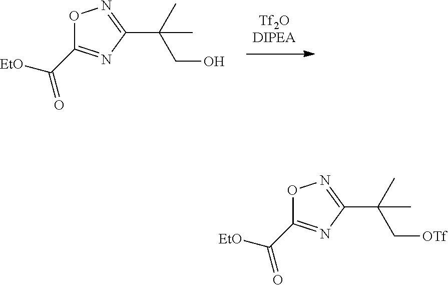 [SMILES: CCOC(=O)c1nc(C(C)(C)CO)no1 ; CCOC(=O)c1nc(C(C)(C)COS(=O)(=O)C(F)(F)F)no1]

To a solution of ethyl 3-(1-hydroxy-2-methylpropan-2-yl)-1,2,4-oxadiazole-5-carboxylate (22.2 g, 104 mmol) and DIPEA (35.4 mL, 207.3 mmol) in DCM (600 mL) at 0° C. was added triflic anhydride (21 mL, 124 mmol) and the mixture was stirred overnight at RT. Additional DCM (200 mL) was added, followed by water (100 mL). The phases were separated, and the organic phase was washed with water (100 mL). The organic phase was dried over Na₂SO₄, filtered, and concentrated under vacuum. The residue was purified by silica gel column chromatography (heptane/EtOAc=5/1 to 3/1) to give ethyl 3-(2-methyl-1-(((trifluoromethyl)sulfonyl)oxy)propan-2-yl)-1,2,4-oxadiazole-5-carboxylate as an orange oil (27.5 g, 76% yield). ¹H NMR (300 MHz, CDCl₃) δ: 4.71 (s, 2H), 4.53 (q, J=7.3 Hz, 2H), 1.56 (s, 6H), 1.43 (t, J=7.4 Hz, 3H).

### 5. Synthesis of ethyl 3-(1-fluoro-2-methylpropan-2-yl)-1,2,4-oxadiazole-5-carboxylate

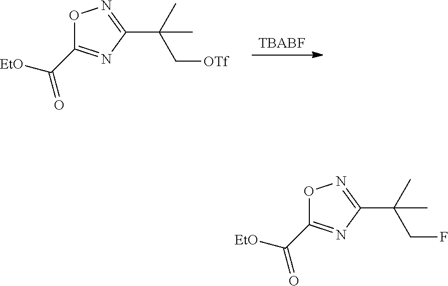 [SMILES: CCOC(=O)c1nc(C(C)(C)COS(=O)(=O)C(F)(F)F)no1 ; "TBABF" ; CCOC(=O)c1nc(C(C)(C)CF)no1]

A solution of ethyl 3-(2-methyl-1-(((trifluoromethyl)sulfonyl)oxy)propan-2-yl)-1,2,4-oxadiazole-5-carboxylate (27.5 g, 79.4 mmol) and tetrabutylammonium hydrogen difluoride (50% in MeCN, 89.0 g, 159 mmol) in THF (700 mL) was stirred at 40° C. for 48 h. The mixture was concentrated under vacuum. The residue was dissolved in EtOAc (700 mL), washed with water (3×100 mL). The organic phase was dried over Na₂SO₄, filtered, and concentrated. The residue was purified by silica gel column chromatography (eluting with heptane/EtOAc=5/1 to 3/1) to give ethyl 3-(1-fluoro-2-methylpropan-2-yl)-1,2,4-oxadiazole-5-carboxylate as a colorless oil (11.3 g, 65% yield). ¹H NMR (300 MHz, CDCl₃) δ: 4.63 (s, 1H), 4.54 (q, J=7.4 Hz, 2H), 4.46 (s, 1H), 1.56-1.38 (m, 9H).

### 6. Synthesis of 3-(1-fluoro-2-methylpropan-2-yl)-1,2,4-oxadiazole-5-carboxylic acid

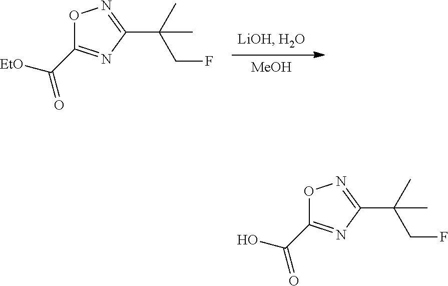

To a cold solution of ethyl 3-(1-fluoro-2-methylpropan-2-yl)-1,2,4-oxadiazole-5-carboxylate (9.70 g, 44.9 mmol) in MeOH (90 mL) was added a solution of LiOH·H₂O (3.76 g, 89.7 mmol) in water (15 mL) and the resulting mixture was stirred for 2 h at RT. MeOH was removed under vacuum and the aqueous residue (cooled with ice bath) was acidified with concentrated HCl until pH=2-3. The mixture was concentrated under vacuum to give a white solid. The residue was dried by co-evaporation with toluene (3×10 mL). The white solid obtained was suspended in 5% MeOH in DCM (100 mL), the mixture was filtered, and the filter cake washed with the same mixture (3×10 mL). The filtrates were combined, dried over anhydrous Na₂SO₄, filtered, and concentrated under vacuum to give 3-(1-fluoro-2-methylpropan-2-yl)-1,2,4-oxadiazole-5-carboxylic acid as white solid (7.0 g, crude), which was carried forward without further purification. LCMS m/z=189.0 [M+H]⁺

### 7. Synthesis of 3-(1-fluoro-2-methylpropan-2-yl)-N-(2-fluoro-4-(6-(1-methyl-1H-pyrazol-4-yl)pyrazolo[1,5-a]pyridin-4-yl)benzyl)-1,2,4-oxadiazole-5-carboxamide

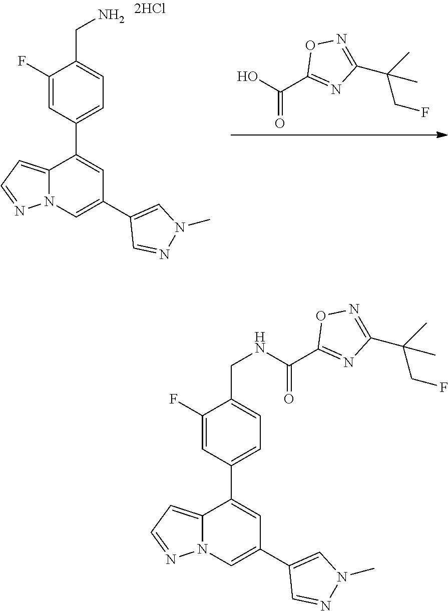 [SMILES: Cn1cc(-c2cc(-c3ccc(CN)c(F)c3)c3ccnn3c2)cn1 ; CC(C)(CF)c1noc(C(=O)O)n1 ; Cn1cc(-c2cc(-c3ccc(CNC(=O)c4nc(C(C)(C)CF)no4)c(F)c3)c3ccnn3c2)cn1]

3-(1-Fluoro-2-methylpropan-2-yl)-N-(2-fluoro-4-(6-(1-methyl-1H-pyrazol-4-yl)pyrazolo[1,5-a]pyridin-4-yl)benzyl)-1,2,4-oxadiazole-5-carboxamide was obtained as a white solid (4.5 mg, 13% yield) from (2-fluoro-4-(6-(1-methyl-1H-pyrazol-4-yl)pyrazolo[1,5-a]pyridin-4-yl)phenyl)methanamine dihydrochloride (Example 67, step 3) and 3-(1-fluoro-2-methylpropan-2-yl)-1,2,4-oxadiazole-5-carboxylic acid following a similar method to that used for Example 1 step 8. The crude material was purified by reverse phase HPLC (Method A, 5-65% gradient). LCMS m/z=492.4 [M+H]^{+ 1}H NMR (500 MHz, DMSO-d₆) δ: 9.98 (t, J=5.8 Hz, 1H), 9.04 (s, 1H), 8.34 (s, 1H), 8.07 (s, 1H), 8.04 (d, J=1.8 Hz, 1H), 7.69-7.52 (m, 4H), 6.75-6.67 (m, 1H), 4.62 (s, 1H), 4.60 (d, J=6.1 Hz, 2H), 4.53 (s, 1H), 3.88 (s, 3H), 1.38 (d, J=1.8 Hz, 6H).

C.4 Preparation of [1,2,4]triazolo[1,5-a]pyrazines

### Example 72: 2-(tert-butyl)-N-((1-(6-(1-methyl-1H-pyrazol-4-yl)-[1,2,4]triazolo[1,5-a]pyrazin-8-yl)piperidin-4-yl)methyl)-2H-tetrazole-5-carboxamide

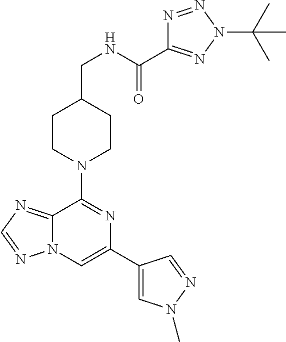 [SMILES: Cn1cc(-c2cn3ncnc3c(N3CCC(CNC(=O)c4nnn(C(C)(C)C)n4)CC3)n2)cn1]

### 1. Preparation of tert-butyl ((1-(6-bromo-[1,2,4]triazolo[1,5-a]pyrazin-8-yl)piperidin-4-yl)methyl)carbamate

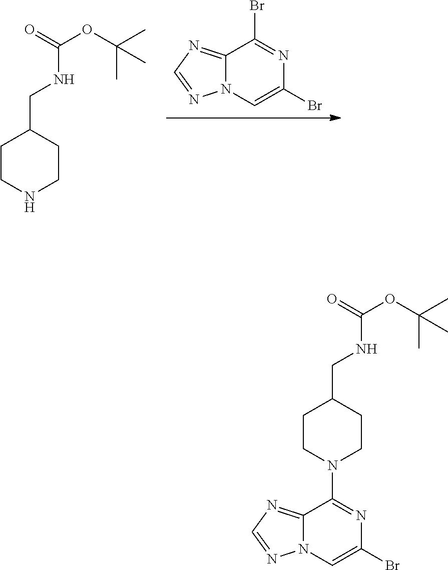

A suspension of tert-butyl N-(4-piperidylmethyl)carbamate (500 mg, 2.33 mmol), 6,8-dibromo-[1,2,4]triazolo[1,5-a]pyrazine (647.52 mg, 2.33 mmol) and DIPEA (602.26 mg, 4.66 mmol) in IPA (10 mL) was stirred at RT for 2 h. After 15 min, a copious white solid formed, so additional IPA (10 mL) was added to allow stirring. The mixture was filtered, the solid washed with IPA and dried to give tert-butyl ((1-(6-bromo-[1,2,4]triazolo[1,5-a]pyrazin-8-yl)piperidin-4-yl)methyl)carbamate (864 mg, 90.2% yield) as a white solid. ¹H NMR (500 MHz, CDCl₃) δ ppm: 8.20 (s, 1H), 7.99 (s, 1H), 5.66-5.30 (br s, 2H), 4.64 (br s, 1H), 3.20-3.01 (m, 4H), 1.95-1.80 (m, 3H), 1.46 (s, 9H), 1.39-1.24 (m, 2H).

### 2. Synthesis of tert-butyl ((1-(6-(1-methyl-1H-pyrazol-4-yl)-[1,2,4]triazolo[1,5-a]pyrazin-8-yl)piperidin-4-yl)methyl)carbamate

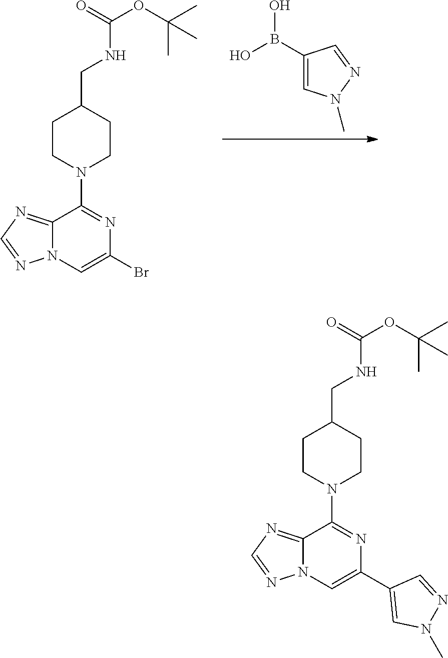

A mixture of tert-butyl ((1-(6-bromo-[1,2,4]triazolo[1,5-a]pyrazin-8-yl)piperidin-4-yl)methyl)carbamate (150 mg, 364.7 μmol), (1-methylpyrazol-4-yl)boronic acid (91.85 mg, 729.4 μmol) and Cs₂CO₃ (356.48 mg, 1.09 mmol) in dry dioxane (3.0 mL) was sparged with N2 for 5 min. RuPhos (34.04 mg, 72.94 μmol) and Pd₂(dba)₃ (33.40 mg, 36.47 μmol) were added and the resulting mixture was heated at 100° C. for 17 h. The cooled mixture was filtered through a pad of Celite® washing through with EtOAc and the filtrate was concentrated in vacuum. The residue was purified by silica gel chromatography (0-100% Heptane in EtOAc) to give tert-butyl ((1-(6-(1-methyl-1H-pyrazol-4-yl)-[1,2,4]triazolo[1,5-a]pyrazin-8-yl)piperidin-4-yl)methyl)carbamate (116 mg, 77.1% yield) as an orange solid. LCMS m/z=413.2 [M+H]⁺

### 3. Synthesis of(1-(6-(1-methyl-1H-pyrazol-4-yl)-[1,2,4]triazolo[1,5-a]pyrazin-8-yl)piperidin-4-yl)methanamine hydrochloride

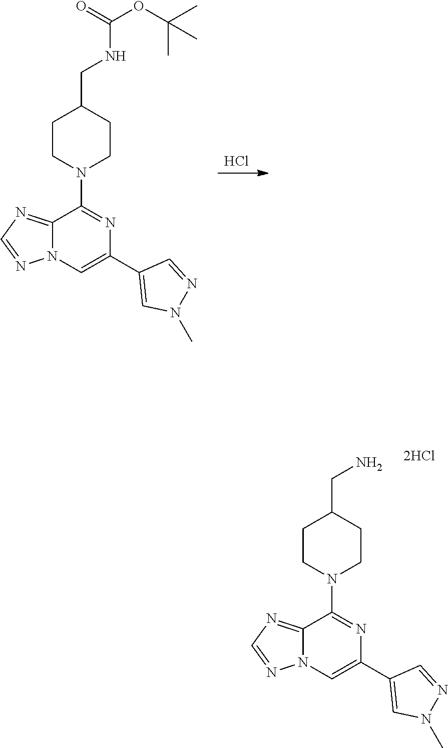 [SMILES: Cn1cc(-c2cn3ncnc3c(N3CCC(CNC(=O)OC(C)(C)C)CC3)n2)cn1 ; Cl ; Cn1cc(-c2cn3ncnc3c(N3CCC(CN)CC3)n2)cn1]

A suspension of tert-butyl ((1-(6-(1-methyl-1H-pyrazol-4-yl)-[1,2,4]triazolo[1,5-a]pyrazin-8-yl)piperidin-4-yl)methyl)carbamate (115 mg, 278.79 μmol) in MeOH (1.0 mL) was treated with 4M HCl in dioxane (975.77 uL) and the reaction stirred at RT for 2 h. The mixture was concentrated to dryness to give (1-(6-(1-methyl-1H-pyrazol-4-yl)-[1,2,4]triazolo[1,5-a]pyrazin-8-yl)piperidin-4-yl)methanamine dihydrochloride as a white solid. LCMS m/z=313.2 [M+H]⁺

### 4. Synthesis of 2-(tert-butyl)-N-((1-(6-(1-methyl-1H-pyrazol-4-yl)-[1,2,4]triazolo[1,5-a]pyrazin-8-yl)piperidin-4-yl)methyl)-2H-tetrazole-5-carboxamide

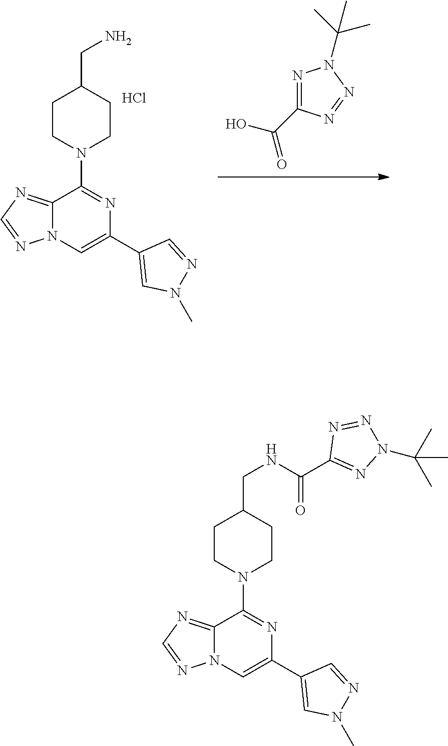 [SMILES: Cn1cc(-c2cn3ncnc3c(N3CCC(CN)CC3)n2)cn1 ; Cl ; CC(C)(C)n1nnc(C(=O)O)n1 ; Cn1cc(-c2cn3ncnc3c(N3CCC(CNC(=O)c4nnn(C(C)(C)C)n4)CC3)n2)cn1]

T3P® (218.91 mg, 344.01 μmol, 50% purity) was added to a solution of 2-tert-butyltetrazole-5-carboxylic acid (43.90 mg, 258.01 μmol), (1-(6-(1-methyl-1H-pyrazol-4-yl)-[1,2,4]triazolo[1,5-a]pyrazin-8-yl)piperidin-4-yl)methanamine hydrochloride (60 mg, 172 μmol) and DIPEA (111.15 mg, 860.02 μmol) in dry DMF (2.0 mL) and the reaction stirred at RT for 2 days. Additional DIPEA (111.15 mg, 860.02 μmol) and T3P® (218.91 mg, 344.01 μmol, 232.89 uL, 50% purity) were added and stirring continued for 2 h. The reaction was quenched with saturated NaHCO₃ solution and extracted with EtOAc. The organic layer was washed with brine (3×) and the combined aqueous layers were extracted with EtOAc. The combined organic layers were dried (MgSO₄), filtered and concentrated in vacuum. The crude was purified by prep HPLC (Method C2, 5-60% gradient) to give 2-(tert-butyl)-N-((1-(6-(1-methyl-1H-pyrazol-4-yl)-[1,2,4]triazolo[1,5-a]pyrazin-8-yl)piperidin-4-yl)methyl)-2H-tetrazole-5-carboxamide (68.40 mg, 81.3% yield) as an orange solid. LCMS m/z=465.2 [M+H]⁺ 1H NMR (500 MHz, CDCl₃) δ ppm: 8.25-8.21 (m, 1H), 8.11-8.08 (m, 1H), 7.87 (s, 1H), 7.84 (s, 1H), 7.34-7.29 (m, 1H), 5.51 (br d, J=13.4 Hz, 2H), 4.00-3.96 (m, 3H), 3.49 (t, J=6.7 Hz, 2H), 3.19-3.11 (m, 2H), 2.14-2.03 (m, 1H), 2.03-1.94 (m, 2H), 1.83-1.81 (m, 9H), 1.52-1.42 (m, 2H).

### Example 73: 5-(tert-butyl)-N-((1-(6-(1-methyl-1H-pyrazol-4-yl)-[1,2,4]triazolo[1,5-a]pyrazin-8-yl)piperidin-4-yl)methyl)-1,2,4-oxadiazole-3-carboxamide

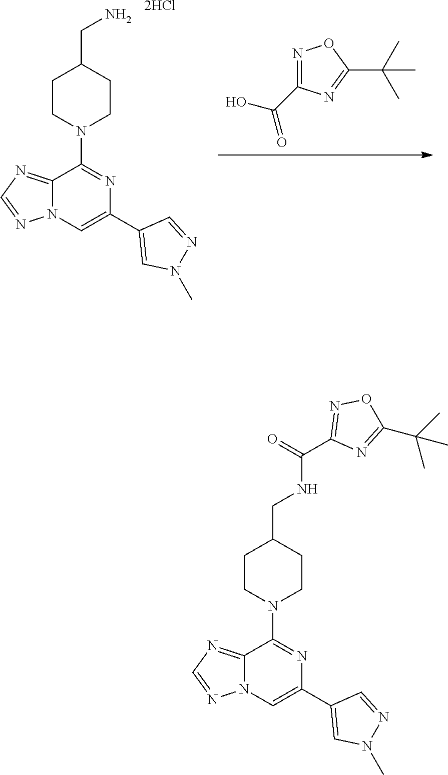 [SMILES: "2HCl" ; Cn1cc(-c2cn3ncnc3c(N3CCC(CN)CC3)n2)cn1 ; CC(C)(C)c1nc(C(=O)O)no1 ; Cn1cc(-c2cn3ncnc3c(N3CCC(CNC(=O)c4noc(C(C)(C)C)n4)CC3)n2)cn1]

To a suspension of (1-(6-(1-methyl-1H-pyrazol-4-yl)-[1,2,4]triazolo[1,5-a]pyrazin-8-yl)piperidin-4-yl)methanamine dihydrochloride (Example 72, step 3, 95.0 mg, 272.34 μmol) and lithium 5-tert-butyl-1,2,4-oxadiazole-3-carboxylate (96.47 mg, 544.68 μmol) in dry DMF (2.0 mL) was added DIPEA (176 mg, 1.36 mmol) followed by T3P® (259.96 mg, 817.02 μmol) and the reaction stirred at RT for 17 h. The reaction was diluted with EtOAc, washed with sat. NaHCO₃, water and brine (2×). The combined aq. layers were re-extracted with EtOAc, the combined organic extracts were dried (MgSO₄), filtered and concentrated in vacuum. The residue was purified by prep-HPLC (Method C2, 5-60% gradient) to give 5-(tert-butyl)-N-((1-(6-(1-methyl-1H-pyrazol-4-yl)-[1,2,4]triazolo[1,5-a]pyrazin-8-yl)piperidin-4-yl)methyl)-1,2,4-oxadiazole-3-carboxamide (79.9 mg, 60% yield) as an off-white solid. LCMS m/z=465.3 [M+H]⁺ 1H NMR (500 MHz, CDCl₃) δ ppm: 8.24 (s, 1H), 8.09 (s, 1H), 7.92 (s, 1H), 7.84 (s, 1H), 7.12 (br t, J=6.1 Hz, 1H), 5.48 (br d, J=13.4 Hz, 2H), 3.97-4.03 (m, 3H), 3.44 (t, J=6.4 Hz, 2H), 3.18-3.08 (m, 2H), 2.13-2.02 (m, 1H), 1.96 (br d, J=12.8 Hz, 2H), 1.48 (s, 9H), 1.47-1.39 (m, 2H).

### Example 74: 1-(tert-butyl)-N-((1-(6-(1-methyl-1H-pyrazol-4-yl)-[1,2,4]triazolo[1,5-a]pyrazin-8-yl)piperidin-4-yl)methyl)-1H-1,2,3-triazole-4-carboxamide

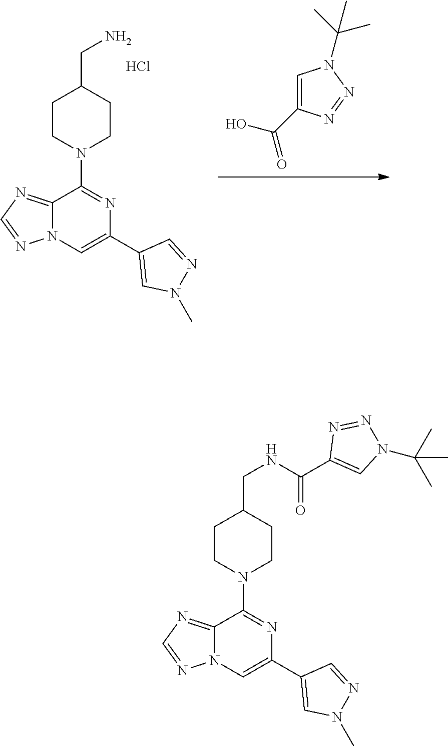 [SMILES: Cn1cc(-c2cn3ncnc3c(N3CCC(CN)CC3)n2)cn1 ; Cl ; CC(C)(C)n1cc(C(=O)O)nn1 ; Cn1cc(-c2cn3ncnc3c(N3CCC(CNC(=O)c4cn(C(C)(C)C)nn4)CC3)n2)cn1]

1-(tert-Butyl)-N-((1-(6-(1-methyl-1H-pyrazol-4-yl)-[1,2,4]triazolo[1,5-a]pyrazin-8-yl)piperidin-4-yl)methyl)-1H-1,2,3-triazole-4-carboxamide was obtained as an off-white solid (55.7 mg, 62.9% yield) from (1-(6-(1-methyl-1H-pyrazol-4-yl)-[1,2,4]triazolo[1,5-a]pyrazin-8-yl)piperidin-4-yl)methanamine dihydrochloride (Example 72, step 3) and 1-tert-butyltriazole-4-carboxylic acid, following the method described in Example 72, step 4. LCMS m/z=464.3 [M+H]⁺ 1H NMR (500 MHz, CDCl₃) δ ppm 8.24 (s, 1H), 8.17-8.13 (m, 1H), 8.09 (s, 1H), 7.90-7.85 (m, 1H), 7.84 (s, 1H), 7.35-7.30 (m, 1H), 5.61-5.43 (m, 2H), 3.99 (s, 3H), 3.44 (t, J=6.4 Hz, 2H), 3.19-3.10 (m, 2H), 2.09-2.02 (m, 1H), 2.02-1.95 (m, 2H), 1.74-1.70 (m, 1H), 1.52-1.40 (m, 2H).

C.5 Preparation of [1,2,4]triazolo[4,3-a]pyridines

### Example 75: 1-(tert-butyl)-N-(4-(7-(1-methyl-1H-pyrazol-4-yl)-[1,2,4]triazolo[4,3-a]pyridin-5-yl)benzyl)-1H-1,2,3-triazole-4-carboxamide

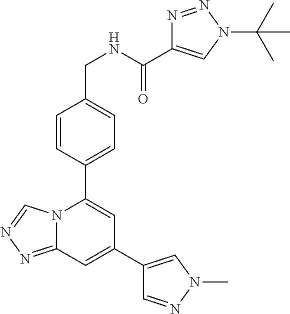 [SMILES: Cn1cc(-c2cc(-c3ccc(CNC(=O)c4cn(C(C)(C)C)nn4)cc3)n3cnnc3c2)cn1]

### 1. Synthesis of 5-chloro-7-(1-methyl-1H-pyrazol-4-yl)-[1,2,4]triazolo[4,3-a]pyridine

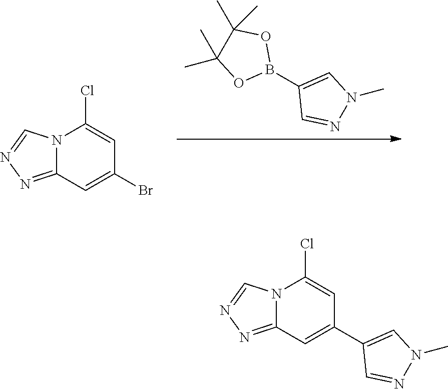

A mixture of 7-bromo-5-chloro-[1,2,4]triazolo[4,3-a]pyridine (112 mg, 0.48 mmol), 1-methyl-4-(4,4,5,5-tetramethyl-1,3,2-dioxaborolan-2-yl)-1H-pyrazole (100 mg, 0.48 mmol), K₂CO₃ (199 mg, 1.4 mmol), and Pd(dppf)Cl₂•DCM (39 mg, 48 μmol) were dissolved in dioxane (3.8 mL) and water (1 mL) and N2 was bubbled through the mixture for 5 mins. The reaction mixture was heated to 100° C. under an atmosphere of N2 and stirred at that temperature for 1 h. The reaction mixture was cooled to ambient temperature and filtered through Celite®. The filtrate was concentrated and the crude material was purified by silica gel column chromatography (3:1 EtOAc/EtOH:heptanes, from 2% to 100%) to give 5-chloro-7-(1-methyl-1H-pyrazol-4-yl)-[1,2,4]triazolo[4,3-a]pyridine as an off-white solid (84 mg, 75% yield). ¹H NMR (500 MHz, MeOH-d₄) δ: 9.27 (s, 1H), 8.25 (s, 1H), 8.06 (s, 1H), 7.87 (s, 1H), 7.54-7.50 (m, 1H), 3.96 (s, 3H).

### 2. Synthesis of tert-butyl (4-(7-(1-methyl-1H-pyrazol-4-yl)-[1,2,4]triazolo[4,3-a]pyridin-5-yl)benzyl)carbamate

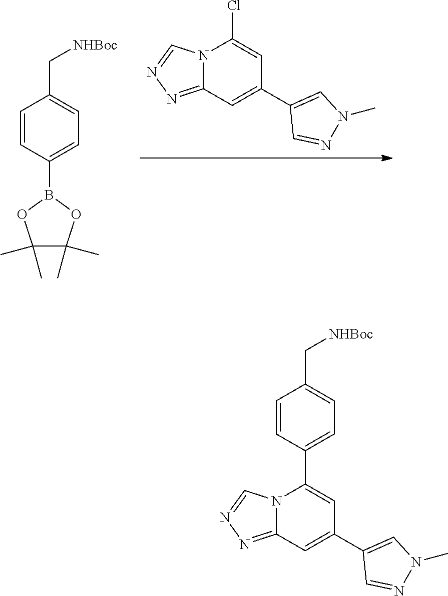

A mixture of tert-butyl (4-(4,4,5,5-tetramethyl-1,3,2-dioxaborolan-2-yl)benzyl)carbamate (120 mg, 0.36 mmol), 5-chloro-7-(1-methyl-1H-pyrazol-4-yl)-[1,2,4]triazolo[4,3-a]pyridine (84 mg, 0.36 mmol), K₂CO₃ (149 mg, 1.1 mmol), and Pd(dppf)Cl₂•DCM (29 mg, 36 μmol) were dissolved in dioxane (2.9 mL) and water (0.7 mL) and N2 was bubbled through the mixture for 5 mins. The reaction mixture was heated to 100° C. under an atmosphere of N2 and was stirred for 2 h. The reaction mixture was cooled to ambient temperature and filtered through Celite®. The filtrate was concentrated and the crude material was purified by silica gel column chromatography ([3:1 EtOAc/EtOH]/heptanes from 2% to 100%) to give tert-butyl (4-(7-(1-methyl-1H-pyrazol-4-yl)-[1,2,4]triazolo[4,3-a]pyridin-5-yl)benzyl)carbamate as a brown solid (129 mg, 89% yield). LCMS m/z=405.1 [M+H]⁺

### 3. Synthesis of (4-(7-(1-methyl-1H-pyrazol-4-yl)-[1,2,4]triazolo[4,3-a]pyridin-5-yl)phenyl)methanamine dihydrochloride

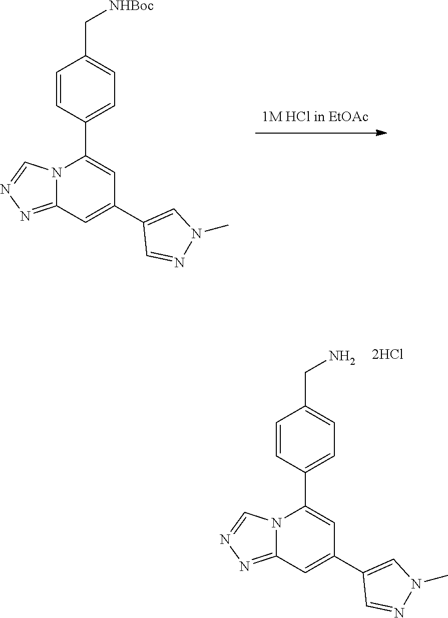 [SMILES: Cn1cc(-c2cc(-c3ccc(CNC(=O)OC(C)(C)C)cc3)n3cnnc3c2)cn1 ; Cn1cc(-c2cc(-c3ccc(CN)cc3)n3cnnc3c2)cn1]

To a solution of tert-butyl (4-(7-(1-methyl-1H-pyrazol-4-yl)-[1,2,4]triazolo[4,3-a]pyridin-5-yl)benzyl)carbamate (125 mg, 309 μmol) in EtOAc (1 mL) was added an HCl solution (1 M in EtOAc, 3.09 mL) and the reaction was stirred for 3 days at ambient temperature. The reaction mixture was concentrated, and the crude material was carried forward without further purification (94 mg, crude). LCMS m/z=305 [M+H]⁺

### 4. Synthesis of 1-(tert-butyl)-N-(4-(7-(1-methyl-1H-pyrazol-4-yl)-[1,2,4]triazolo[4,3-a]pyridin-5-yl)benzyl)-1H-1,2,3-triazole-4-carboxamide

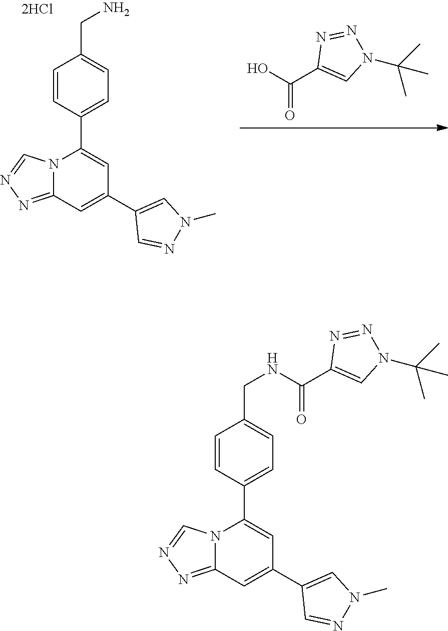 [SMILES: Cn1cc(-c2cc(-c3ccc(CN)cc3)n3cnnc3c2)cn1 ; CC(C)(C)n1cc(C(=O)O)nn1 ; Cn1cc(-c2cc(-c3ccc(CNC(=O)c4cn(C(C)(C)C)nn4)cc3)n3cnnc3c2)cn1]

To a solution of 1-(tert-butyl)-1H-1,2,3-triazole-4-carboxylic acid (13 mg, 75 μmol) in THF (2 mL) in an ice water cooling bath was added TEA (20 mg, 201 μmol) and HATU (38 mg, 101 μmol). The reaction mixture was stirred at 0° C. for 10 min before (4-(7-(1-methyl-1H-pyrazol-4-yl)-[1,2,4]triazolo[4,3-a]pyridin-5-yl)phenyl)methanamine dihydrochloride (19 mg, 50 μmol) was added. The reaction mixture was warmed to 23° C. and stirred for 24 h. Water (5 mL) was added to quench the reaction, then EtOAc (5 mL) added and the layers were separated. The aqueous phase was extracted EtOAc (2×5 mL). The organic phases were combined, washed with brine, dried over Na₂SO₄, filtered, and concentrated. The crude material was purified by reverse phase HPLC (Method C1, 5-45% gradient) to give 1-(tert-butyl)-N-(4-(7-(1-methyl-1H-pyrazol-4-yl)-[1,2,4]triazolo[4,3-a]pyridin-5-yl)benzyl)-1H-1,2,3-triazole-4-carboxamide as a white solid (14.7 mg, 51% yield). LCMS m/z=456.2 [M+H]^{+ 1}H NMR (500 MHz, DMSO-d₆) δ: 9.29-9.16 (m, 2H), 8.71 (s, 1H), 8.52 (s, 1H), 8.22 (s, 1H), 8.02 (s, 1H), 7.85-7.76 (m, 2H), 7.57 (d, J=8.5 Hz, 2H), 7.41 (s, 1H), 4.58 (d, J=6.7 Hz, 2H), 3.89 (s, 3H), 1.68-1.61 (m, 9H).

### Example 76: N-(4-(7-(1-methyl-1H-pyrazol-4-yl)-[1,2,4]triazolo[4,3-a]pyridin-5-yl)benzyl)-5-(1-methylcyclopropyl)-1,2,4-oxadiazole-3-carboxamide

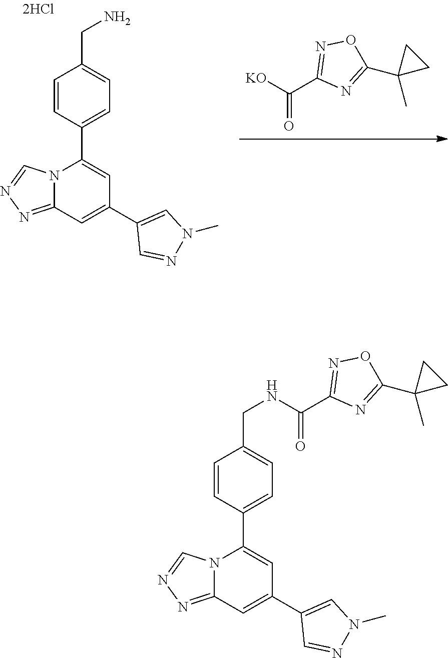

N-(4-(7-(1-methyl-1H-pyrazol-4-yl)-[1,2,4]triazolo[4,3-a]pyridin-5-yl)benzyl)-5-(1-methylcyclopropyl)-1,2,4-oxadiazole-3-carboxamide was obtained (13.8 mg, 48% yield) as a white solid, from potassium 5-(1-methylcyclopropyl)-1,2,4-oxadiazole-3-carboxylate and (4-(7-(1-methyl-1H-pyrazol-4-yl)-[1,2,4]triazolo[4,3-a]pyridin-5-yl)phenyl)methanamine dihydrochloride (Example 75, step 3) following the method used in Example 75, step 4. LCMS m/z=455.2 [M+H]^{+ 1}H NMR (500 MHz, DMSO-d₆) δ: 9.57 (t, J=6.1 Hz, 1H), 9.18 (s, 1H), 8.51 (s, 1H), 8.22 (s, 1H), 8.02 (s, 1H), 7.82 (d, J=8.5 Hz, 2H), 7.57 (d, J=8.5 Hz, 2H), 7.39 (s, 1H), 4.56 (d, J=6.7 Hz, 2H), 3.95-3.84 (m, 3H), 1.59-1.51 (m, 3H), 1.44-1.34 (m, 2H), 1.23-1.13 (m, 2H).

### Example 77: 1-(tert-butyl)-N-(4-(7-(1-methyl-1H-pyrazol-4-yl)-[1,2,4]triazolo[4,3-a]pyridin-5-yl)benzyl)-1H-pyrazole-4-carboxamide

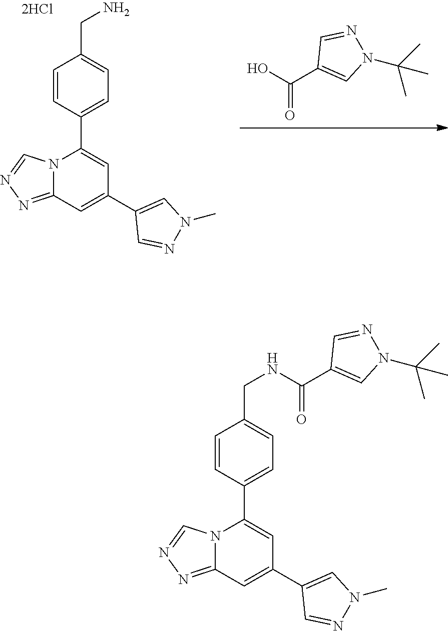 [SMILES: Cn1cc(-c2cc(-c3ccc(CN)cc3)n3cnnc3c2)cn1 ; CC(C)(C)n1cc(C(=O)O)cn1 ; Cn1cc(-c2cc(-c3ccc(CNC(=O)c4cnn(C(C)(C)C)c4)cc3)n3cnnc3c2)cn1]

1-(tert-Butyl)-N-(4-(7-(1-methyl-1H-pyrazol-4-yl)-[1,2,4]triazolo[4,3-a]pyridin-5-yl)benzyl)-1H-pyrazole-4-carboxamide was obtained as a white solid (19.6 mg, 68% yield) from 1-(tert-butyl)-1H-pyrazole-4-carboxylic acid and (4-(7-(1-methyl-1H-pyrazol-4-yl)-[1,2,4]triazolo[4,3-a]pyridin-5-yl)phenyl)methanamine dihydrochloride (Example 75, step 3), following the method used in Example 75, step 4. LCMS m/z=455.2 [M+H]⁺. ¹H NMR (500 MHz, DMSO-d₆) δ: 9.18 (s, 1H), 8.72 (t, J=6.1 Hz, 1H), 8.52 (s, 1H), 8.32 (s, 1H), 8.23 (s, 1H), 8.02 (s, 1H), 7.92 (s, 1H), 7.81 (d, J=8.5 Hz, 2H), 7.55 (d, J=7.9 Hz, 2H), 7.40 (s, 1H), 4.54 (d, J=6.1 Hz, 2H), 3.89 (s, 3H), 1.59-1.49 (m, 9H).

### Example 78: 5-(tert-butyl)-N-(2-methyl-4-(7-(1-methyl-1H-pyrazol-4-yl)imidazo[1,2-c]pyrimidin-5-yl)benzyl)-1,2,4-oxadiazole-3-carboxamide

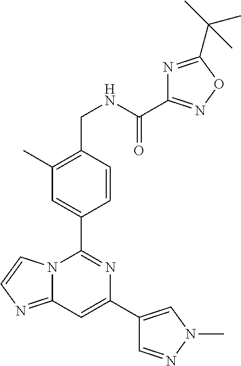 [SMILES: Cc1cc(-c2nc(-c3cnn(C)c3)cc3nccn23)ccc1CNC(=O)c1noc(C(C)(C)C)n1]

A mixture of 5-(tert-butyl)-N-(2-methyl-4-(4,4,5,5-tetramethyl-1,3,2-dioxaborolan-2-yl)benzyl)-1,2,4-oxadiazole-3-carboxamide (Step 1, Example 55, 100 mg, 250.4 μmol), 5-chloro-7-(1-methyl-1H-pyrazol-4-yl)imidazo[1,2-c]pyrimidine (Step 4, Example 56, 58.52 mg, 250.4 μmol), K₂CO₃ (103.84 mg, 751.3 μmol) and Pd(dppf)Cl₂ (9.16 mg, 12.5 μmol) were dissolved in dioxane (2.0 mL) and water (0.5 mL) and N2 was bubbled through the mixture for 5 min. The reaction mixture was heated to 100° C. under N2 and stirred at 100° C. overnight. The cooled reaction was concentrated, dissolved in MeOH, and filtered through at 0.45 μM syringe filter. The filtrate was concentrated and purified by HPLC (Method C3, 5-75%) to afford 5-(tert-butyl)-N-(2-methyl-4-(7-(1-methyl-1H-pyrazol-4-yl)imidazo[1,2-c]pyrimidin-5-yl)benzyl)-1,2,4-oxadiazole-3-carboxamide (70 mg, 56.4% yield). LCMS m/z=471.3 [M+H]⁺. ¹H NMR (500 MHz, DMSO-d₆) δ ppm 9.54 (t, J=6.10 Hz, 1H) 8.33 (s, 1H) 8.10 (s, 1H) 7.93-7.90 (m, 1H) 7.83 (s, 1H) 7.82-7.78 (m, 2H), 7.64-7.61 (m, 1H) 7.48 (d, J=8.55 Hz, 1H) 4.56 (d, J=6.10 Hz, 2H) 3.89 (s, 3H) 2.46 (s, 3H) 1.44 (s, 9H).

### Example 79: 5-(tert-butyl)-N-(3-fluoro-2-methyl-4-(7-(1-methyl-1H-pyrazol-4-yl)imidazo[1,2-c]pyrimidin-5-yl)benzyl)-1,2,4-oxadiazole-3-carboxamide

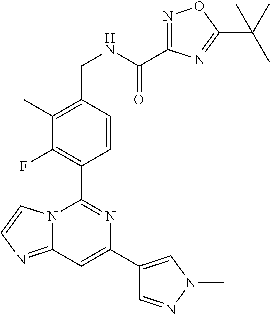 [SMILES: Cc1c(CNC(=O)c2noc(C(C)(C)C)n2)ccc(-c2nc(-c3cnn(C)c3)cc3nccn23)c1F]

### 1. Preparation of (4-bromo-3-fluoro-2-methylphenyl)methanamine hydrochloride

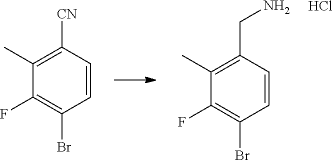

To a solution of 4-bromo-3-fluoro-2-methylbenzonitrile (62.2 g, 290 mmol) in THF (150 mL) was added BH₃·THF (49.9 g, 581 mmol) and the solution was stirred at 70° C. overnight. The reaction was quenched by cautiously adding MeOH at 0° C., the mixture was concentrated, the residue was dissolved in DCM (1 L) and 36% aqueous HCl (20 mL) slowly added. The mixture was stirred for 30 min, the resulting solid filtered off, washed with DCM and TBME and dried under vacuum to give (4-bromo-3-fluoro-2-methylphenyl)methanamine hydrochloride (58.6 g, 79%) as a grey solid. LCMS m/z=218.2 [M+H]⁺.

### 2. Preparation of tert-butyl (4-bromo-3-fluoro-2-methylbenzyl)carbamate

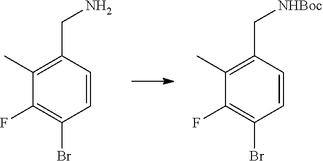 [SMILES: Cc1c(CN)ccc(Br)c1F ; Cc1c(CNC(=O)OC(C)(C)C)ccc(Br)c1F]

To a suspension of 4-bromo-3-fluoro-2-methylphenyl)methanamine hydrochloride (63.0 g, 247 mmol) in DCM (450 mL), was added TEA (75.1 g, 742 mmol) and (Boc)₂O (64.8 g, 297 mmol) and the reaction was stirred at rt overnight. The mixture was washed with water and brine, dried over Na₂SO₄ and concentrated. The crude product was used as such in the next step.

### 3. Preparation of (tert-butyl (3-fluoro-2-methyl-4-(4,4,5,5-tetramethyl-1,3,2-dioxaborolan-2-yl)benzyl)carbamate

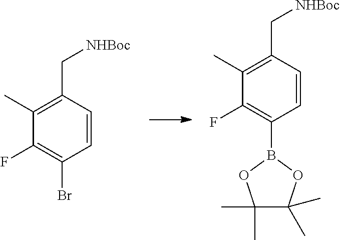 [SMILES: Cc1c(CNC(=O)OC(C)(C)C)ccc(Br)c1F ; Cc1c(CNC(=O)OC(C)(C)C)ccc(B2OC(C)(C)C(C)(C)O2)c1F]

A solution of tert-butyl (4-bromo-3-fluoro-2-methylbenzyl)carbamate (47.4 g, 149 mmol), bis(pinacolato)diboron (45.4 g, 179 mmol) and KOAc (29.3 g, 298 mmol) in dioxane (600 mL) was stirred under N2 at rt for 10 min. PdCl₂(dppf)•DCM (12.2 g, 14.9 mmol) was added and the reaction mixture was stirred at 90° C. for 16 h. The mixture was filtered through Celite®, the filtrate was diluted with EtOAc, then washed with water, aq. NaHCO₃ and brine. The organic layer was dried over Na₂SO₄ and concentrated in vacuo. The crude product was purified by column chromatography (heptane: EtOAc gradient 0 to 30% EtOAc) to give impure product. The solid was triturated with hexane, the solid filtered off and dried to give (tert-butyl (3-fluoro-2-methyl-4-(4,4,5,5-tetramethyl-1,3,2-dioxaborolan-2-yl)benzyl)carbamate. 42.3 g, 78%.

### 4. Preparation of (3-fluoro-2-methyl-4-(4,4,5,5-tetramethyl-1,3,2-dioxaborolan-2-yl)phenyl)methanamine hydrochloride

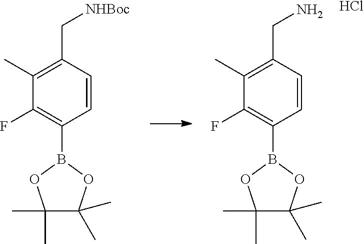 [SMILES: Cl ; Cc1c(CNC(=O)OC(C)(C)C)ccc(B2OC(C)(C)C(C)(C)O2)c1F ; Cc1c(CN)ccc(B2OC(C)(C)C(C)(C)O2)c1F]

tert-Butyl (3-fluoro-2-methyl-4-(4,4,5,5-tetramethyl-1,3,2-dioxaborolan-2-yl)benzyl)carbamate (30.0 g, 82.1 mmol) was suspended in 4 M HCl in dioxane (62 mL, 246 mmol) and the mixture was stirred for 1 h at rt. The mixture was concentrated in vacuum and azeotroped with toluene (5×50 mL) to give (3-fluoro-2-methyl-4-(4,4,5,5-tetramethyl-1,3,2-dioxaborolan-2-yl)phenyl)methanamine hydrochloride (25.8 g, crude) as a light pink solid.

### 5. Preparation of (3-fluoro-2-methyl-4-(4,4,5,5-tetramethyl-1,3,2-dioxaborolan-2-yl)phenyl)methanamine

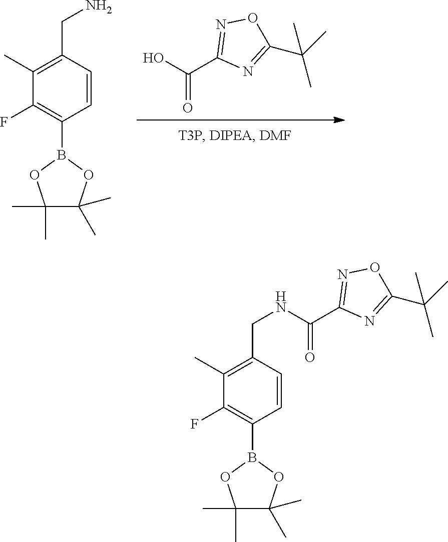 [SMILES: Cc1c(CN)ccc(B2OC(C)(C)C(C)(C)O2)c1F ; CC(C)(C)c1nc(C(=O)O)no1 ; Cc1c(CNC(=O)c2noc(C(C)(C)C)n2)ccc(B2OC(C)(C)C(C)(C)O2)c1F]

DIPEA (45 mL, 257 mmol) was added to a suspension of (3-fluoro-2-methyl-4-(4,4,5,5-tetramethyl-1,3,2-dioxaborolan-2-yl)phenyl)methanamine hydrochloride (25.8 g, 85.6 mmol) and 5-(tert-butyl)-1,2,4-oxadiazole-3-carboxylic acid (21.8 g, 128 mmol) in anhydrous DMF (500 mL) and the mixture was cooled to 0° C. T3P® (64.7 mL, 111 mmol) was added and the resulting mixture was allowed to warm to rt and stirred for 2 h. The reaction was quenched with water (300 mL) and EtOAc (300 mL) was added. The layers were separated and the aqueous layer was extracted with EtOAc (3×100 mL). The combined organic layers were washed with water (3×50 mL), sat. aq. solution of NaHCO₃ (50 mL) and with brine (50 mL) then dried over Na₂SO₄, filtered and concentrated in vacuum to give a beige oil. This was purified by silica gel column chromatography (heptanes: EtOAc, 0-20% gradient) twice, to give (3-fluoro-2-methyl-4-(4,4,5,5-tetramethyl-1,3,2-dioxaborolan-2-yl)phenyl)methanamine as a thick pale-yellow oil, which slowly solidified upon standing (11.2 g, 31%).

### 6. Preparation of 5-(tert-butyl)-N-(3-fluoro-2-methyl-4-(7-(1-methyl-1H-pyrazol-4-yl)imidazo[1,2-c]pyrimidin-5-yl)benzyl)-1,2,4-oxadiazole-3-carboxamide

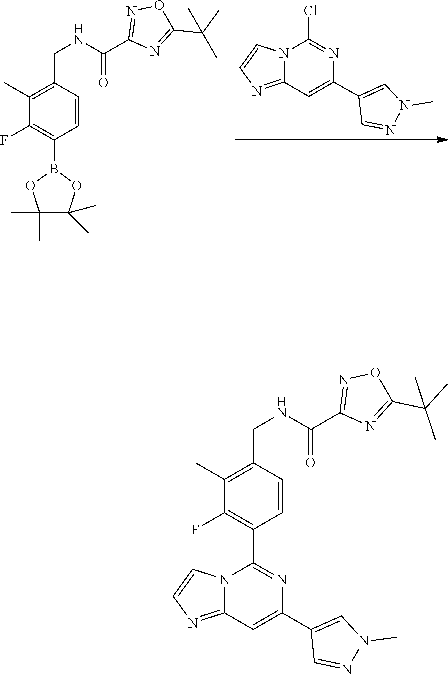

A mixture of 5-(tert-butyl)-N-(3-fluoro-2-methyl-4-(4,4,5,5-tetramethyl-1,3,2-dioxaborolan-2-yl)benzyl)-1,2,4-oxadiazole-3-carboxamide (75.0 mg, 179.74 μmol), 5-chloro-7-(1-methylpyrazol-4-yl)imidazo[1,2-c]pyrimidine (42.00 mg, 179.74 μmol), K₂CO₃ (74.5 mg, 539.2 μmol) and Pd(dppf)Cl₂ (6.58 mg, 8.99 μmol) were dissolved in dioxane (1.44 mL) and water (0.36 mL) and N2 was bubbled through the mixture for 5 min. The reaction mixture was heated to 100° C. under an atmosphere of N2 and stirred at 100° C. for 3 h. The reaction was concentrated and purified via column chromatography (gradient elution 0-100% [3:1 EtOAc:EtOH]:heptane) to afford 5-(tert-butyl)-N-(3-fluoro-2-methyl-4-(7-(1-methyl-1H-pyrazol-4-yl)imidazo[1,2-c]pyrimidin-5-yl)benzyl)-1,2,4-oxadiazole-3-carboxamide (88 mg, 99.2% yield) as a light yellow solid. LCMS m/z=489.2 [M+H]⁺. 1H NMR (400 MHz, DMSO-d₆) δ ppm 9.58 (t, J=5.77 Hz, 1H) 8.31 (s, 1H) 8.08 (s, 1H) 7.91 (s, 1H) 7.66-7.60 (m, 2H) 7.57 (br s, 1H) 7.35 (d, J=8.03 Hz, 1H) 4.59 (d, J=6.02 Hz, 2H) 3.87 (s, 3H) 2.35 (d, J=2.01 Hz, 3H) 1.44 (s, 9H).

### Example 80: 5-(tert-butyl)-N-(2-methyl-4-(6-(1-methyl-1H-pyrazol-4-yl)pyrazolo[1,5-a]pyrazin-4-yl)benzyl)-1,2,4-oxadiazole-3-carboxamide

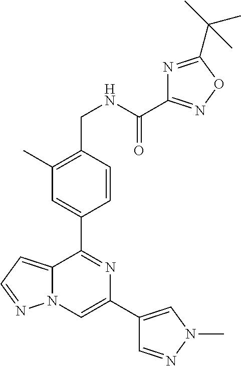 [SMILES: Cc1cc(-c2nc(-c3cnn(C)c3)cn3nccc23)ccc1CNC(=O)c1noc(C(C)(C)C)n1]

5-(tert-butyl)-N-(2-methyl-4-(6-(1-methyl-1H-pyrazol-4-yl)pyrazolo[1,5-a]pyrazin-4-yl)benzyl)-1,2,4-oxadiazole-3-carboxamide was obtained as a white solid, 74 mg, 82.1% yield, from 5-(tert-butyl)-N-(2-methyl-4-(4,4,5,5-tetramethyl-1,3,2-dioxaborolan-2-yl)benzyl)-1,2,4-oxadiazole-3-carboxamide (Step 1, Example 55) and 4-chloro-6-(1-methyl-1H-pyrazol-4-yl)pyrazolo[1,5-a]pyrazine (Example 1, step 5), following the procedure described in Example 79, step 6. LCMS m/z=471.2 [M+H]⁺. ¹H NMR (400 MHz, DMSO-d₆) δ ppm 9.50 (t, J=5.90 Hz, 1H) 9.12 (d, J=1.00 Hz, 1H) 8.34 (s, 1H) 8.18 (d, J=2.51 Hz, 1H) 8.11 (d, J=0.75 Hz, 1H) 7.95 (d, J=5.02 Hz, 2H) 7.45 (d, J=8.53 Hz, 1H) 7.15 (dd, J=2.51, 1.00 Hz, 1H) 4.55 (d, J=6.27 Hz, 2H) 3.91 (s, 3H) 2.48 (s, 3H) 1.44 (s, 9H).

### Example 81: 5-(tert-butyl)-N-(3-fluoro-2-methyl-4-(6-(1-methyl-1H-pyrazol-4-yl)pyrazolo[1,5-a]pyrazin-4-yl)benzyl)-1,2,4-oxadiazole-3-carboxamide

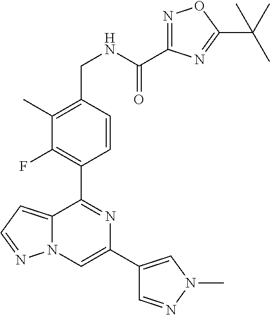 [SMILES: Cc1c(CNC(=O)c2noc(C(C)(C)C)n2)ccc(-c2nc(-c3cnn(C)c3)cn3nccc23)c1F]

5-(tert-Butyl)-N-(3-fluoro-2-methyl-4-(6-(1-methyl-1H-pyrazol-4-yl)pyrazolo[1,5-a]pyrazin-4-yl)benzyl)-1,2,4-oxadiazole-3-carboxamide was obtained as a light yellow solid, 81 mg, 90.4% yield, from 5-(tert-butyl)-N-(3-fluoro-2-methyl-4-(4,4,5,5-tetramethyl-1,3,2-dioxaborolan-2-yl)benzyl)-1,2,4-oxadiazole-3-carboxamide (Example 79, step 5) and 4-chloro-6-(1-methylpyrazol-4-yl)pyrazolo[1,5-a]pyrazine (Example 1, step 5), following the procedure described in Example 79, step 6. LCMS m/z=489.2 [M+H]⁺ 1H NMR (400 MHz, DMSO-d₆) δ ppm 9.55 (t, J=6.15 Hz, 1H) 9.19 (d, J=1.00 Hz, 1H) 8.27 (s, 1H) 8.14 (d, J=2.26 Hz, 1H) 8.07 (d, J=0.75 Hz, 1H) 7.59 (t, J=7.53 Hz, 1H) 7.30 (d, J=8.03 Hz, 1H) 6.75 (t, J=2.13 Hz, 1H) 4.58 (d, J=5.77 Hz, 2H) 3.88 (s, 3H) 2.35 (d, J=2.01 Hz, 3H) 1.44 (s, 9H).

### Example 82: 5-(tert-butyl)-N-(2-chloro-4-(pyrazolo[1,5-a]pyrazin-4-yl)benzyl)-1,2,4-oxadiazole-3-carboxamide

### 1. Preparation of tert-butyl (2-chloro-4-(pyrazolo[1,5-a]pyrazin-4-yl)benzyl)carbamate

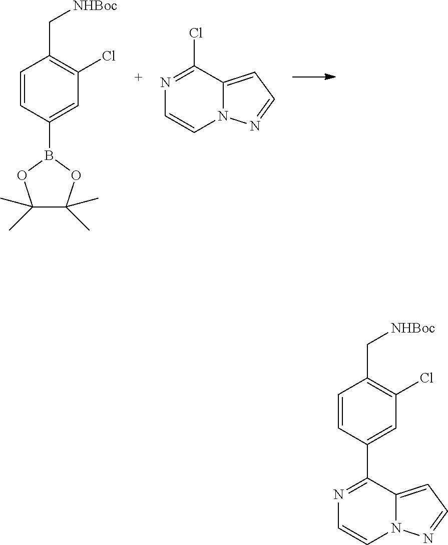 [SMILES: CC(C)(C)OC(=O)NCc1ccc(B2OC(C)(C)C(C)(C)O2)cc1Cl ; Clc1nccn2nccc12 ; CC(C)(C)OC(=O)NCc1ccc(-c2nccn3nccc23)cc1Cl]

To a solution of tert-butyl (2-chloro-4-(4,4,5,5-tetramethyl-1,3,2-dioxaborolan-2-yl)benzyl)carbamate (359.1 mg, 0.977 mmol) in dioxane (30 mL) and H₂O (5 mL) was added 4-chloropyrazolo[1,5-a]pyrazine (150 mg, 0.977 mmol) and K₂CO₃ (270 mg, 1.95 mmol) at 20° C. Pd(dppf)Cl₂ (71.5 mg, 97.7 μmol) was added and the reaction was stirred at 90° C. for 3 h under N2. The mixture was filtered and concentrated under vacuum to give the crude, which was purified by chromatography column on silica gel (PE/EtOAc=3/1) to give tert-butyl (2-chloro-4-(pyrazolo[1,5-a]pyrazin-4-yl)benzyl)carbamate (200 mg, 57.1% yield) as a yellow solid.

### 2. Preparation of(2-chloro-4-(pyrazolo[1,5-a]pyrazin-4-yl)phenyl)methanamine dihydrochloride

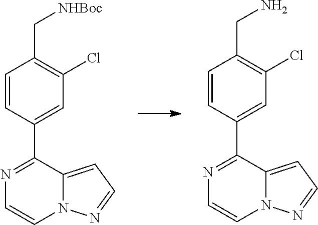 [SMILES: CC(C)(C)OC(=O)NCc1ccc(-c2nccn3nccc23)cc1Cl ; NCc1ccc(-c2nccn3nccc23)cc1Cl]

A solution of tert-butyl (2-chloro-4-(pyrazolo[1,5-a]pyrazin-4-yl)benzyl)carbamate (200 mg, 0.557 mmol) in 4M HCl/EtOAc (20 mL) was stirred at 20° C. for 1 h. The mixture was concentrated under vacuum to give (2-chloro-4-pyrazolo[1,5-a]pyrazin-4-yl-phenyl)methanamine dihydrochloride (150 mg), as a white solid, which was used in the next step directly without further purification.

### 3. Preparation of 5-(tert-butyl)-1,2,4-oxadiazole-3-carbonyl chloride

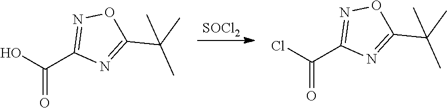

A solution of 5-tert-butyl-1,2,4-oxadiazole-3-carboxylic acid (200 mg, 1.18 mmol) in DCM (10 mL) and DMF (0.2 mL) was added thionyl chloride (559.3 mg, 4.70 mmol) at 15° C. and the reaction was stirred at 15° C. for 0.5 h. The mixture was filtered and concentrated under vacuum to give 5-tert-butyl-1,2,4-oxadiazole-3-carbonyl chloride (200 mg, crude) as white solid.

### 4. Preparation of 5-(tert-butyl)-N-(2-chloro-4-(pyrazolo[1,5-a]pyrazin-4-yl)benzyl)-1,2,4-oxadiazole-3-carboxamide

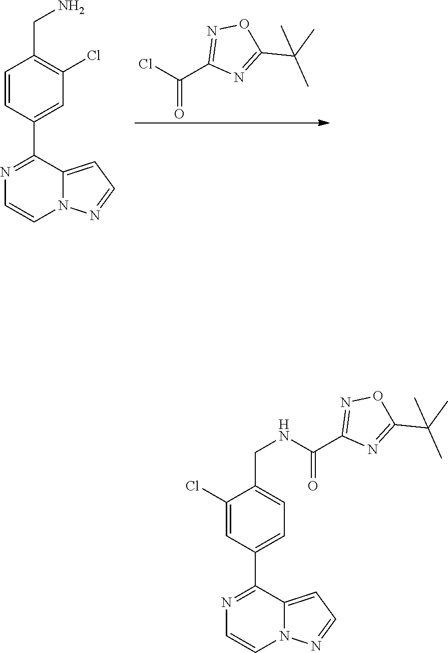 [SMILES: NCc1ccc(-c2nccn3nccc23)cc1Cl ; CC(C)(C)c1nc(C(=O)Cl)no1 ; CC(C)(C)c1nc(C(=O)NCc2ccc(-c3nccn4nccc34)cc2Cl)no1]

To a solution of (2-chloro-4-(pyrazolo[1,5-a]pyrazin-4-yl)phenyl)methanamine dihydrochloride (140 mg, 474.3 μmol) in DCM (30 mL) was added DIPEA (122.6 mg, 948.6 μmol) at 20° C. 5-tert-Butyl-1,2,4-oxadiazole-3-carbonyl chloride (Example 82 step 3, 134.2 mg, 711.5 μmol) was added and the reaction mixture was stirred at 20° C. for 1 h. The mixture was poured into water (50 mL) and extracted with DCM (50 mL×3). The combined organic layers were washed with brine (100 mL), dried over Na₂SO₄, filtered and concentrated under vacuum. The crude product was purified by Prep-HPLC (Method D, 48-68% gradient) to give 5-(tert-butyl)-N-(2-chloro-4-(pyrazolo[1,5-a]pyrazin-4-yl)benzyl)-1,2,4-oxadiazole-3-carboxamide (88.9 mg, 45.6% yield) as a white solid. LCMS m/z=411.0 [M+H]⁺ 1H NMR: (400 MHz, DMSO-d₆) δ=9.63-9.59 (m, 1H), 9.47 (m, 1H), 8.26 (s, 1H), 8.09-8.03 (m, 3H), 7.57-7.54 (m, 1H), 7.21 (s, 1H), 4.62 (d, J=6.0 Hz, 2H), 1.44 (s, 9H).

### Example 83. 5-(tert-butyl)-N-(3-fluoro-4-(6-(1-methyl-1H-pyrazol-4-yl)pyrazolo[1,5-a]pyrazin-4-yl)benzyl)-1,2,4-oxadiazole-3-carboxamide

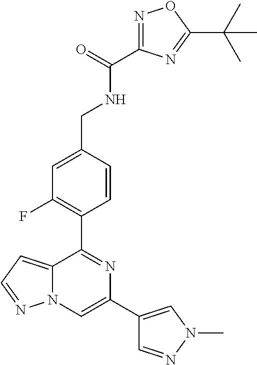 [SMILES: Cn1cc(-c2cn3nccc3c(-c3ccc(CNC(=O)c4noc(C(C)(C)C)n4)cc3F)n2)cn1]

### 1. Preparation of tert-butyl (3-fluoro-4-(4,4,5,5-tetramethyl-1,3,2-dioxaborolan-2-yl)benzyl)carbamate

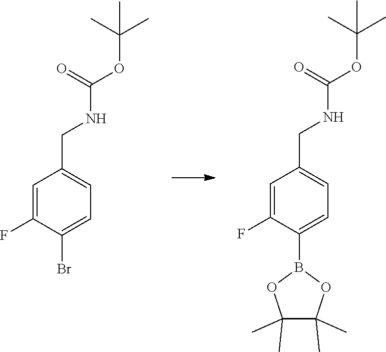 [SMILES: CC(C)(C)OC(=O)NCc1ccc(Br)c(F)c1 ; CC(C)(C)OC(=O)NCc1ccc(B2OC(C)(C)C(C)(C)O2)c(F)c1]

KOAc (8.13 g, 82.86 mmol) and Pd(dppf)Cl₂ DCM (2.26 g, 2.76 mmol) were added to a solution of tert-butyl (4-bromo-3-fluorobenzyl)carbamate (8.40 g, 27.6 mmol) and (BPin)₂ (14.03 g, 55.2 mmol) in dioxane (150 mL) and the reaction was stirred at 90° C. under N2 for 12 h. The mixture was concentrated to dryness to give crude product, which was purified by column chromatography on silica gel (PE/EtOAc=20/1) to give tert-butyl (3-fluoro-4-(4,4,5,5-tetramethyl-1,3,2-dioxaborolan-2-yl)benzyl)carbamate (9.00 g, 83.5% yield) as a white solid. LCMS m/z=296.0 [M-CH₄+H]⁺.

### 2. Preparation of tert-butyl (3-fluoro-4-(6-(1-methyl-1H-pyrazol-4-yl)pyrazolo[1,5-a]pyrazin-4-yl)benzyl)carbamate

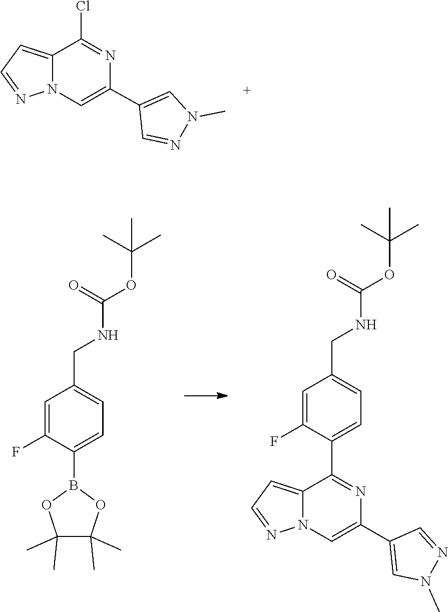 [SMILES: Cn1cc(-c2cn3nccc3c(Cl)n2)cn1 ; CC(C)(C)OC(=O)NCc1ccc(B2OC(C)(C)C(C)(C)O2)c(F)c1 ; Cn1cc(-c2cn3nccc3c(-c3ccc(CNC(=O)OC(C)(C)C)cc3F)n2)cn1]

K₂CO₃ (472.7 mg, 3.42 mmol) and Pd(dppf)Cl₂•DCM (93.1 mg, 114 μmol) were added to a solution of tert-butyl (3-fluoro-4-(4,4,5,5-tetramethyl-1,3,2-dioxaborolan-2-yl)benzyl)carbamate (400 mg, 1.14 mmol) and 4-chloro-6-(1-methyl-1H-pyrazol-4-yl)pyrazolo[1,5-a]pyrazine (Example 1, step 5, 266.1 mg, 1.14 mmol) in dioxane (30 mL) and water (3 mL) and the mixture was stirred at 90° C. under N2 for 5 h. The mixture was concentrated to dryness and the crude product was purified by column chromatography (PE/EtOAc=1:4) to give tert-butyl (3-fluoro-4-(6-(1-methyl-1H-pyrazol-4-yl)pyrazolo[1,5-a]pyrazin-4-yl)benzyl)carbamate (400 mg, 74.8% yield) as a brown solid.

### 3. Preparation of(3-fluoro-4-(6-(1-methyl-1H-pyrazol-4-yl)pyrazolo[1,5-a]pyrazin-4-yl)phenyl)methanamine dihydrochloride

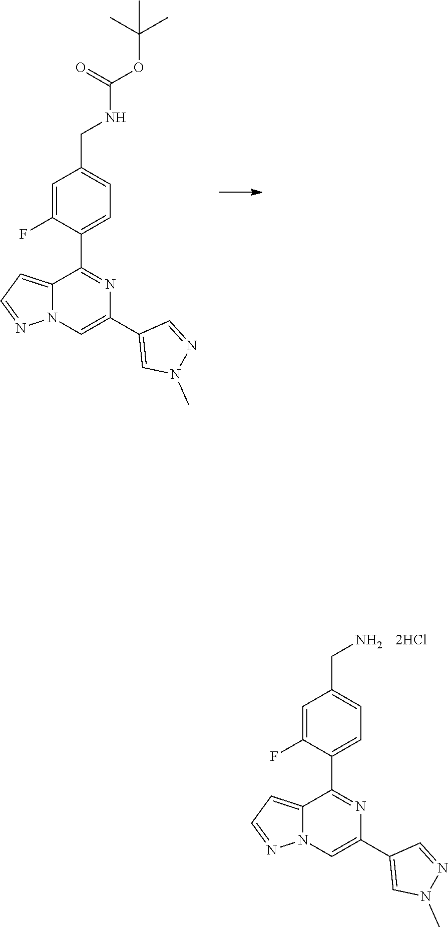 [SMILES: Cn1cc(-c2cn3nccc3c(-c3ccc(CNC(=O)OC(C)(C)C)cc3F)n2)cn1 ; Cn1cc(-c2cn3nccc3c(-c3ccc(CN)cc3F)n2)cn1 ; "2HCl"]

A solution of tert-butyl (3-fluoro-4-(6-(1-methyl-1H-pyrazol-4-yl)pyrazolo[1,5-a]pyrazin-4-yl)benzyl)carbamate (400 mg, 946.9 μmol) in HCl/EtOAc (4 M, 20 mL) was stirred at 20° C. for 1 h. The mixture was concentrated under vacuum to give (3-fluoro-4-(6-(1-methyl-1H-pyrazol-4-yl)pyrazolo[1,5-a]pyrazin-4-yl)phenyl)methanamine dihydrochloride (340 mg, 90.1% yield), as a yellow solid. It was used for the next step without purification.

### 4. Preparation of 5-(tert-butyl)-N-(3-fluoro-4-(6-(1-methyl-1H-pyrazol-4-yl)pyrazolo[1,5-a]pyrazin-4-yl)benzyl)-1,2,4-oxadiazole-3-carboxamide

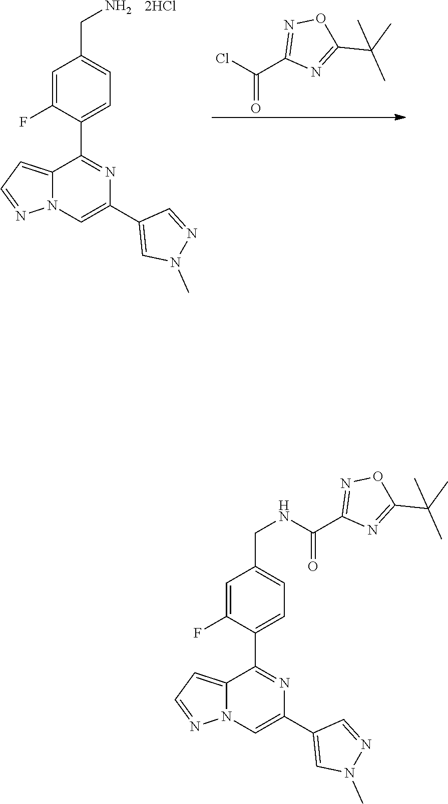 [SMILES: Cn1cc(-c2cn3nccc3c(-c3ccc(CN)cc3F)n2)cn1 ; "2HCl" ; CC(C)(C)c1nc(C(=O)Cl)no1 ; Cn1cc(-c2cn3nccc3c(-c3ccc(CNC(=O)c4noc(C(C)(C)C)n4)cc3F)n2)cn1]

To a solution of (3-fluoro-4-(6-(1-methyl-1H-pyrazol-4-yl)pyrazolo[1,5-a]pyrazin-4-yl)phenyl)methanamine dihydrochloride (80 mg, 223 μmol) in DCM (50 mL) was added DIPEA (86.45 mg, 668.9 μmol). 5-tert-Butyl-1,2,4-oxadiazole-3-carbonyl chloride (Example 82, step 3, 63.08 mg, 334.5 μmol) was added and the mixture was stirred at 20° C. for 1 h. The mixture was concentrated under vacuum and the crude was purified by Prep-HPLC (Method D, 43-63% gradient) to give 5-(tert-butyl)-N-(3-fluoro-4-(6-(1-methyl-1H-pyrazol-4-yl)pyrazolo[1,5-a]pyrazin-4-yl)benzyl)-1,2,4-oxadiazole-3-carboxamide (30 mg, 28.4% yield) as a brown solid. LCMS m/z=475.1 [M+H]⁺ 1H NMR (500 MHz, DMSO-d₆) δ: 9.63 (t, J=5.0 Hz, 1H), 9.20 (s, 1H), 8.28 (s, 1H), 8.15 (s, 1H), 8.07 (s, 1H), 7.78 (t, J=5.0 Hz, 1H), 7.37 (t, J=5.0 Hz, 2H), 6.77 (s, 1H), 4.57 (d, J=5.0 Hz, 2H), 3.88 (s, 3H), 1.43 (s, 9H).

### Example 84. 5-(tert-butyl)-N-(2,5-difluoro-4-(6-(1-methyl-1H-pyrazol-4-yl)pyrazolo[1,5-a]pyrazin-4-yl)benzyl)-1,2,4-oxadiazole-3-carboxamide

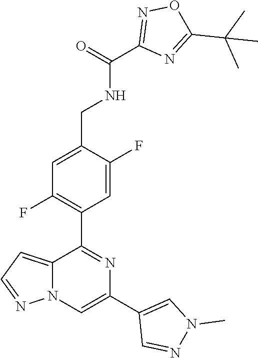 [SMILES: Cn1cc(-c2cn3nccc3c(-c3cc(F)c(CNC(=O)c4noc(C(C)(C)C)n4)cc3F)n2)cn1]

### 1. Preparation of tert-butyl (2,5-difluoro-4-(4,4,5,5-tetramethyl-1,3,2-dioxaborolan-2-yl)benzyl)carbamate

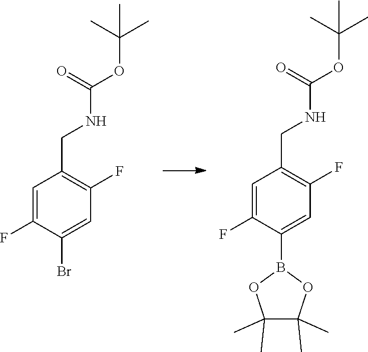 [SMILES: CC(C)(C)OC(=O)NCc1cc(F)c(Br)cc1F ; CC(C)(C)OC(=O)NCc1cc(F)c(B2OC(C)(C)C(C)(C)O2)cc1F]

tert-Butyl (2,5-difluoro-4-(4,4,5,5-tetramethyl-1,3,2-dioxaborolan-2-yl)benzyl)carbamate was obtained, 8.0 g, 89.5% yield, as a white solid, from tert-butyl (4-bromo-2,5-difluorobenzyl)carbamate and (BPin)₂, following the procedure described in Example 83, Step 1. LCMS m/z=314.0 [M-CH₄+H]⁺

### 2. Preparation of tert-butyl (2,5-difluoro-4-(6-(1-methyl-1H-pyrazol-4-yl)pyrazolo[1,5-a]pyrazin-4-yl)benzyl)carbamate

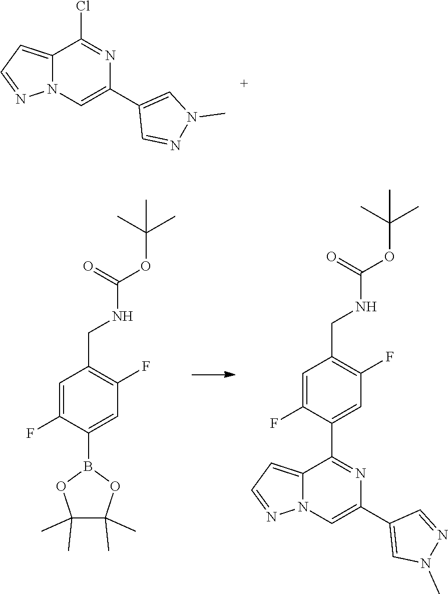 [SMILES: Cn1cc(-c2cn3nccc3c(Cl)n2)cn1 ; CC(C)(C)OC(=O)NCc1cc(F)c(B2OC(C)(C)C(C)(C)O2)cc1F ; Cn1cc(-c2cn3nccc3c(-c3cc(F)c(CNC(=O)OC(C)(C)C)cc3F)n2)cn1]

K₂CO₃ (47.32 mg, 0.342 mmol) and Pd(dppf)Cl₂ (12.53 mg, 17.12 μmol) were added to a solution of tert-butyl (2,5-difluoro-4-(4,4,5,5-tetramethyl-1,3,2-dioxaborolan-2-yl)benzyl)carbamate (82.17 mg, 0.223 mmol) and 4-chloro-6-(1-methyl-1H-pyrazol-4-yl)pyrazolo[1,5-a]pyrazine (Example 1, step 5, 40.0 mg, 0.172 mmol) in dioxane (8 mL) and water (1 mL) and the mixture was stirred at 90° C. under N2 for 2 h. The mixture was filtered and concentrated to dryness and the crude product was purified by column chromatography (PE/EtOAc=1:0 to 1/2) to give tert-butyl (2,5-difluoro-4-(6-(1-methyl-1H-pyrazol-4-yl)pyrazolo[1,5-a]pyrazin-4-yl)benzyl)carbamate (40 mg, 53.1% yield) as a white solid. LCMS m/z=441.1 [M+H]⁺

### 3. Preparation of(2,5-difluoro-4-(6-(1-methyl-1H-pyrazol-4-yl)pyrazolo[1,5-a]pyrazin-4-yl)phenyl)methanamine dihydrochloride

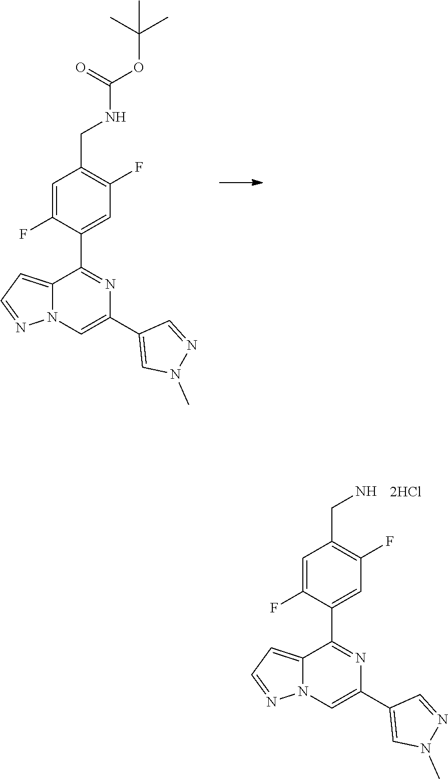 [SMILES: Cn1cc(-c2cn3nccc3c(-c3cc(F)c(CNC(=O)OC(C)(C)C)cc3F)n2)cn1 ; Cn1cc(-c2cn3nccc3c(-c3cc(F)c(CN)cc3F)n2)cn1 ; "2HCl"]

A solution of tert-butyl (2,5-difluoro-4-(6-(1-methyl-1H-pyrazol-4-yl)pyrazolo[1,5-a]pyrazin-4-yl)benzyl)carbamate (650 mg, 1.48 mmol) in HCl/EtOAc (4 M, 15 mL) and DCM (10 mL) was stirred at 20° C. for 1 h. The mixture was concentrated under vacuum to give (2,5-difluoro-4-(6-(1-methyl-1H-pyrazol-4-yl)pyrazolo[1,5-a]pyrazin-4-yl)phenyl)methanamine dihydrochloride (555 mg), as a white solid. It was used for the next step without purification. LCMS m/z=341.0 [M+H]⁺

### 4. Preparation of 5-(tert-butyl)-N-(2,5-difluoro-4-(6-(1-methyl-1H-pyrazol-4-yl)pyrazolo[1,5-a]pyrazin-4-yl)benzyl)-1,2,4-oxadiazole-3-carboxamide

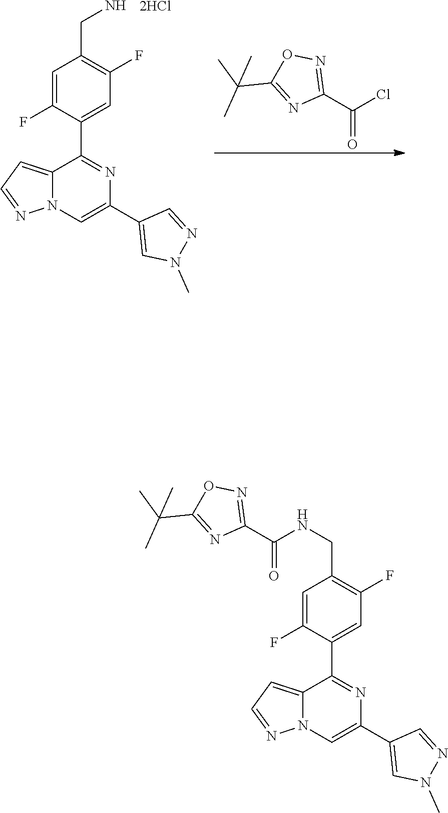 [SMILES: Cn1cc(-c2cn3nccc3c(-c3cc(F)c(CN)cc3F)n2)cn1 ; "2HCl" ; CC(C)(C)c1nc(C(=O)Cl)no1 ; Cn1cc(-c2cn3nccc3c(-c3cc(F)c(CNC(=O)c4noc(C(C)(C)C)n4)cc3F)n2)cn1]

To a solution of (2,5-difluoro-4-(6-(1-methyl-1H-pyrazol-4-yl)pyrazolo[1,5-a]pyrazin-4-yl)phenyl)methanamine dihydrochloride (80 mg, 235.1 μmol) in DCM (50 mL) was added DIPEA (151.9 mg, 1.18 mmol). 5-tert-Butyl-1,2,4-oxadiazole-3-carbonyl chloride (Example 82, step 3, 88.67 mg, 470.1 μmol) was added and the mixture was stirred at 20° C. for 1 h. The mixture was concentrated under vacuum and the crude was purified by Prep-HPLC (Method E, 40-70% gradient) to give 5-(tert-butyl)-N-(2,5-difluoro-4-(6-(1-methyl-1H-pyrazol-4-yl)pyrazolo[1,5-a]pyrazin-4-yl)benzyl)-1,2,4-oxadiazole-3-carboxamide (29 mg, 25.1% yield) as a white solid. LCMS m/z=493.0 [M+H]⁺ 1H NMR (400 MHz, DMSO-d₆) δ: 9.55 (t, J=6.0 Hz, 1H), 9.20 (s, 1H), 8.27 (s, 1H), 8.14 (d, J=2.4 Hz, 1H), 8.05 (s, 1H), 7.67-7.62 (m, 1H), 7.43-7.38 (m, 1H), 6.79 (s, 1H), 4.57 (d, J=6.0 Hz, 2H), 3.86 (s, 3H), 1.41 (s, 9H)

### Example 85: 5-(tert-butyl)-N-(2-methyl-4-(pyrazolo[1,5-a]pyrazin-4-yl)benzyl)-1,2,4-oxadiazole-3-carboxamide

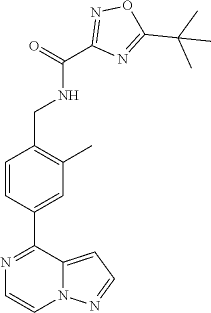 [SMILES: Cc1cc(-c2nccn3nccc23)ccc1CNC(=O)c1noc(C(C)(C)C)n1]

### 1. Preparation of tert-butyl (2-methyl-4-(pyrazolo[1,5-a]pyrazin-4-yl)benzyl)carbamate

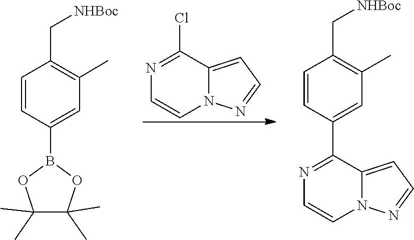

To a solution of tert-butyl (2-methyl-4-(4,4,5,5-tetramethyl-1,3,2-dioxaborolan-2-yl)benzyl)carbamate (Step 1, Example 14, 226.1 mg, 651.2 μmol) in dioxane (20 mL) and H₂O (2 mL) was added 4-chloropyrazolo[1,5-a]pyrazine (100 mg, 651.2 μmol) and K₂CO₃ (180 mg, 1.30 mmol) at 20° C. Pd(dppf)Cl₂ (47.65 mg, 65.1 μmol) was added and the reaction was stirred at 90° C. for 3 h under N2. The cooled mixture was filtered and concentrated under vacuum. The crude product was purified by column chromatography on silica gel (PE/EtOAc=4/1) to give tert-butyl (2-methyl-4-(pyrazolo[1,5-a]pyrazin-4-yl)benzyl)carbamate (150 mg, 68.1% yield) as a yellow solid. LCMS m/z=339.0 [M+H]⁺

### 2. Preparation of(2-methyl-4-(pyrazolo[1,5-a]pyrazin-4-yl)phenyl)methanamine dihydrochloride

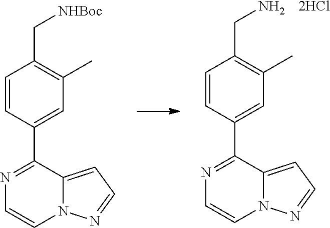 [SMILES: Cc1cc(-c2nccn3nccc23)ccc1CNC(=O)OC(C)(C)C ; Cc1cc(-c2nccn3nccc23)ccc1CN ; "2HCl"]

A solution of tert-butyl (2-methyl-4-(pyrazolo[1,5-a]pyrazin-4-yl)benzyl)carbamate (150 mg, 443.3 μmol) in HCl/EtOAc (20 mL) was stirred at 20° C. for 1 h. The mixture was concentrated under vacuum to give (2-methyl-4-(pyrazolo[1,5-a]pyrazin-4-yl)phenyl)methanamine dihydrochloride (110 mg, crude) as a white solid, which was used in the next step without further purification. LCMS m/z=239.0 [M+H]⁺

### 3. Preparation of 5-(tert-butyl)-N-(2-methyl-4-(pyrazolo[1,5-a]pyrazin-4-yl)benzyl)-1,2,4-oxadiazole-3-carboxamide

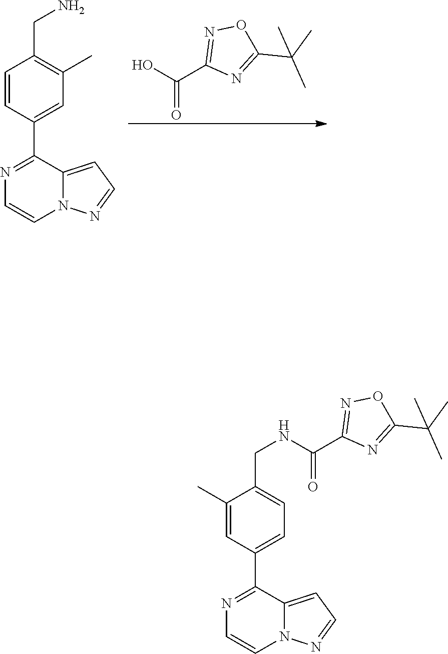 [SMILES: Cc1cc(-c2nccn3nccc23)ccc1CN ; CC(C)(C)c1nc(C(=O)O)no1 ; Cc1cc(-c2nccn3nccc23)ccc1CNC(=O)c1noc(C(C)(C)C)n1]

To a solution of (2-methyl-4-(pyrazolo[1,5-a]pyrazin-4-yl)phenyl)methanamine dihydrochloride (100 mg, 364 μmol) in DCM (30 mL) was added DIPEA (94.1 mg, 727.9 μmol) and HATU (276.8 mg, 0.728 mmol). 5-(tert-Butyl)-1,2,4-oxadiazole-3-carboxylic acid (103 mg, 546 μmol) was added and the reaction was stirred at 20° C. for 1 h. The mixture was poured into water (50 mL) and extracted with DCM (50 mL×3). The combined organic layers were washed with brine (100 mL), dried over Na₂SO₄, filtered and concentrated under vacuum to give the crude, which was purified by Prep-HPLC (Method D, 45-65% gradient) to give 5-(tert-butyl)-N-(2-methyl-4-(pyrazolo[1,5-a]pyrazin-4-yl)benzyl)-1,2,4-oxadiazole-3-carboxamide (55.7 mg, 39.2% yield) as a white solid. LCMS m/z=391.1 [M+H]⁺. ¹H NMR: (400 MHz, DMSO-d₆) δ=9.53-9.49 (m, 1H), 8.78 (d, J=4.4 Hz, 1H), 8.24 (d, J=2.4 Hz, 1H), 8.00 (d, J=4.4 Hz, 1H), 7.88-7.86 (m, 2H), 7.45-7.42 (m, 1H), 7.19 (d, J=1.6 Hz, 1H), 4.54 (d, J=6.0 Hz, 2H), 2.45 (s, 3H), 1.43 (s, 9H).

### Example 86. 5-(tert-butyl)-N-(2-(difluoromethyl)-4-(6-(1-methyl-1H-pyrazol-4-yl)pyrazolo[1,5-a]pyrazin-4-yl)benzyl)-1,2,4-oxadiazole-3-carboxamide

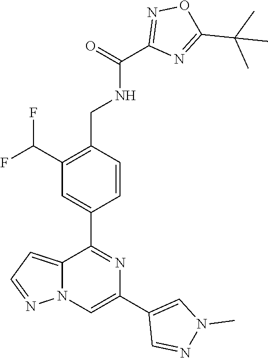 [SMILES: Cn1cc(-c2cn3nccc3c(-c3ccc(CNC(=O)c4noc(C(C)(C)C)n4)c(C(F)F)c3)n2)cn1]

### 1. Preparation of tert-butyl (2-(difluoromethyl)-4-(4,4,5,5-tetramethyl-1,3,2-dioxaborolan-2-yl)benzyl)carbamate

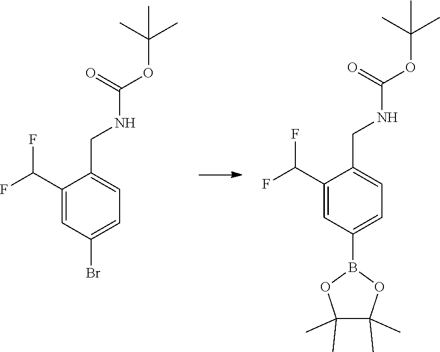 [SMILES: CC(C)(C)OC(=O)NCc1ccc(Br)cc1C(F)F ; CC(C)(C)OC(=O)NCc1ccc(B2OC(C)(C)C(C)(C)O2)cc1C(F)F]

To a solution of tert-butyl (4-bromo-2-(difluoromethyl)benzyl)carbamate (4.20 g, 12.49 mmol) in dioxane (120 mL) was added (BPin)₂ (3.33 g, 13.11 mmol), KOAc (3.68 g, 37.47 mmol) and Pd(dppf)Cl₂ (9.14 g, 12.49 mmol) and the reaction was stirred at 85° C. for 16 h. The mixture was concentrated to dryness to give a residue which was purified by silica gel chromatography (PE:EtOAc, 100:0 to 85:5) to give tert-butyl (2-(difluoromethyl)-4-(4,4,5,5-tetramethyl-1,3,2-dioxaborolan-2-yl)benzyl)carbamate (4.30 g, 62.9% yield) as clear semi-solid.

### 2. Preparation of tert-butyl (2-(difluoromethyl)-4-(6-(1-methyl-1H-pyrazol-4-yl)pyrazolo[1,5-a]pyrazin-4-yl)benzyl)carbamate

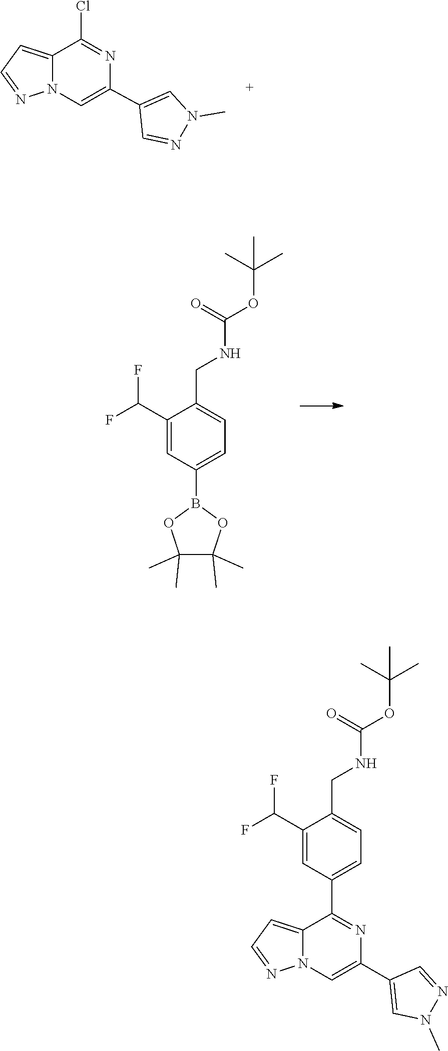 [SMILES: Cn1cc(-c2cn3nccc3c(Cl)n2)cn1 ; CC(C)(C)OC(=O)NCc1ccc(B2OC(C)(C)C(C)(C)O2)cc1C(F)F ; Cn1cc(-c2cn3nccc3c(-c3ccc(CNC(=O)OC(C)(C)C)c(C(F)F)c3)n2)cn1]

tert-Butyl (2-(difluoromethyl)-4-(6-(1-methyl-1H-pyrazol-4-yl)pyrazolo[1,5-a]pyrazin-4-yl)benzyl)carbamate was obtained as a yellow solid, 800 mg, 74% yield, from tert-butyl (2-(difluoromethyl)-4-(4,4,5,5-tetramethyl-1,3,2-dioxaborolan-2-yl)benzyl)carbamate and 4-chloro-6-(1-methyl-1H-pyrazol-4-yl)pyrazolo[1,5-a]pyrazine (Example 1, step 5), following the procedure described in Example 83, step 2. LCMS m/z=455.1 [M+H]⁺

### 3. Preparation of (2-(difluoromethyl)-4-(6-(1-methyl-1H-pyrazol-4-yl)pyrazolo[1,5-a]pyrazin-4-yl)phenyl)methanamine dihydrochloride

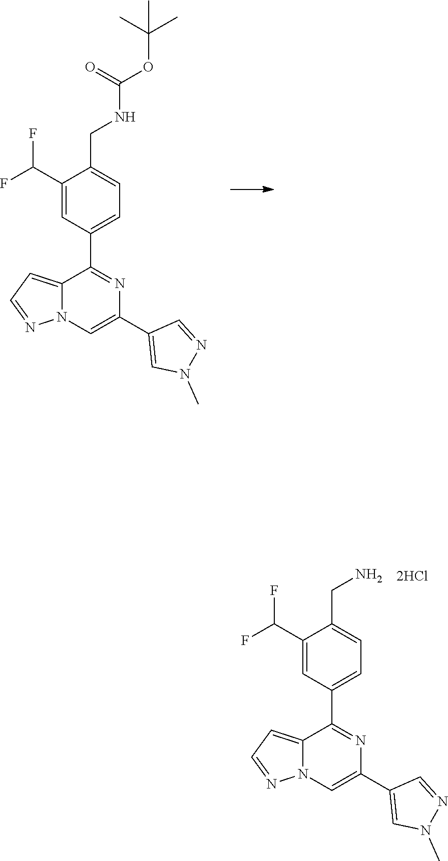 [SMILES: Cn1cc(-c2cn3nccc3c(-c3ccc(CNC(=O)OC(C)(C)C)c(C(F)F)c3)n2)cn1 ; Cn1cc(-c2cn3nccc3c(-c3ccc(CN)c(C(F)F)c3)n2)cn1 ; "2HCl"]

(2-(Difluoromethyl)-4-(6-(1-methyl-1H-pyrazol-4-yl)pyrazolo[1,5-a]pyrazin-4-yl)phenyl)methanamine dihydrochloride was obtained as a yellow solid, 700 mg, crude from tert-butyl (2-(difluoromethyl)-4-(6-(1-methyl-1H-pyrazol-4-yl)pyrazolo[1,5-a]pyrazin-4-yl)benzyl)carbamate, following the procedure described in Example 85, step 2. LCMS m/z=355.0 [M+H]⁺

### 4. Preparation of 5-(tert-butyl)-N-(2-(difluoromethyl)-4-(6-(1-methyl-1H-pyrazol-4-yl)pyrazolo[1,5-a]pyrazin-4-yl)benzyl)-1,2,4-oxadiazole-3-carboxamide

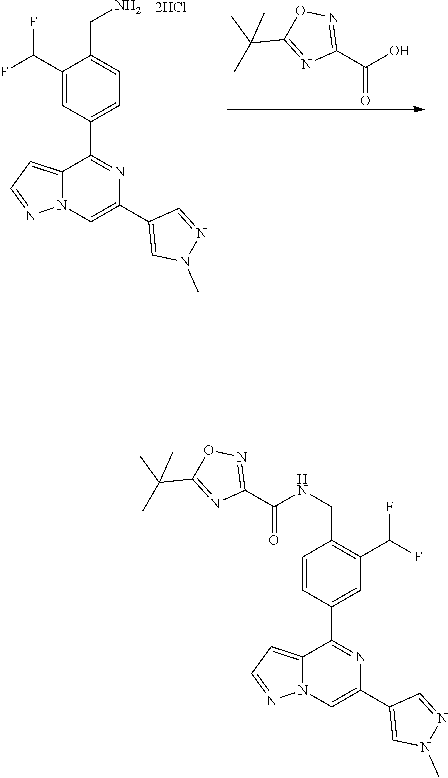 [SMILES: Cn1cc(-c2cn3nccc3c(-c3ccc(CN)c(C(F)F)c3)n2)cn1 ; "2HCl" ; CC(C)(C)c1nc(C(=O)O)no1 ; Cn1cc(-c2cn3nccc3c(-c3ccc(CNC(=O)c4noc(C(C)(C)C)n4)c(C(F)F)c3)n2)cn1]

5-(tert-Butyl)-N-(2-(difluoromethyl)-4-(6-(1-methyl-1H-pyrazol-4-yl)pyrazolo[1,5-a]pyrazin-4-yl)benzyl)-1,2,4-oxadiazole-3-carboxamide was obtained as a yellow solid, 50.2 mg, 43% yield, from (2-(difluoromethyl)-4-(6-(1-methyl-1H-pyrazol-4-yl)pyrazolo[1,5-a]pyrazin-4-yl)phenyl)methanamine dihydrochloride and 5-(tert-butyl)-1,2,4-oxadiazole-3-carboxylic acid following a similar procedure to that described in Example 85, step 3. LCMS m/z=507.1 [M+H]⁺ 1H NMR (400 MHz, DMSO-d₆) δ: 9.64-9.60 (m, 1H), 9.15 (s, 1H), 8.31-8.26 (m, 3H), 8.19 (s, 1H), 8.09 (s, 1H), 7.63 (d, J=8.0 Hz, 1H), 7.14-7.13 (m, 1H), 7.12 (s, 1H), 4.71 (d, J=6.0 Hz, 2H), 3.88 (s, 3H), 1.41 (s, 9H).

### Example 87. 2-(tert-butyl)-N-(2,3-difluoro-4-(6-(1-methyl-1H-pyrazol-4-yl)pyrazolo[1,5-a]pyrazin-4-yl)benzyl)-2H-tetrazole-5-carboxamide

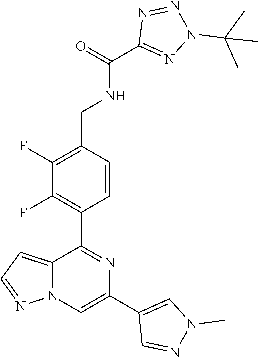 [SMILES: Cn1cc(-c2cn3nccc3c(-c3ccc(CNC(=O)c4nnn(C(C)(C)C)n4)c(F)c3F)n2)cn1]

### 1. Preparation of tert-butyl (2,3-difluoro-4-(6-(1-methyl-1H-pyrazol-4-yl)pyrazolo[1,5-a]pyrazin-4-yl)benzyl)carbamate

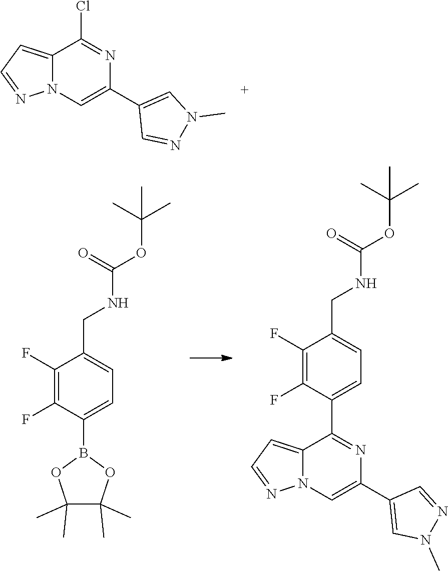 [SMILES: Cn1cc(-c2cn3nccc3c(Cl)n2)cn1 ; CC(C)(C)OC(=O)NCc1ccc(B2OC(C)(C)C(C)(C)O2)c(F)c1F ; Cn1cc(-c2cn3nccc3c(-c3ccc(CNC(=O)OC(C)(C)C)c(F)c3F)n2)cn1]

tert-Butyl (2,3-difluoro-4-(6-(1-methyl-1H-pyrazol-4-yl)pyrazolo[1,5-a]pyrazin-4-yl)benzyl)carbamate was obtained as a yellow solid, 100 mg, 27.9% yield, from tert-butyl (2,3-difluoro-4-(4,4,5,5-tetramethyl-1,3,2-dioxaborolan-2-yl)benzyl)carbamate and 4-chloro-6-(1-methyl-1H-pyrazol-4-yl)pyrazolo[1,5-a]pyrazine (Example 1, step 5), following the procedure described in Example 83, step 2. LCMS m/z=441.1 [M+H]⁺.

### 2. Preparation of (2,3-difluoro-4-(6-(1-methyl-1H-pyrazol-4-yl)pyrazolo[1,5-a]pyrazin-4-yl)phenyl)methanamine dihydrochloride

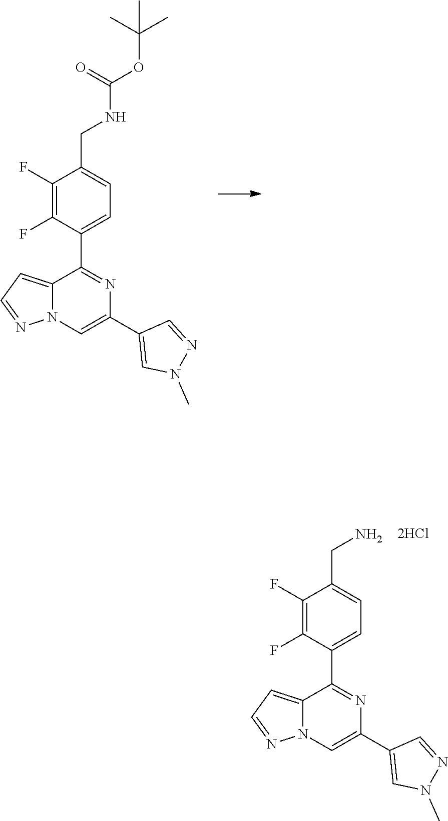 [SMILES: Cn1cc(-c2cn3nccc3c(-c3ccc(CNC(=O)OC(C)(C)C)c(F)c3F)n2)cn1 ; Cn1cc(-c2cn3nccc3c(-c3ccc(CN)c(F)c3F)n2)cn1]

To a solution of tert-butyl (2,3-difluoro-4-(6-(1-methyl-1H-pyrazol-4-yl)pyrazolo[1,5-a]pyrazin-4-yl)benzyl)carbamate (100 mg, 227 μmol) in DCM (2 mL) was added HCl/EtOAc (15 mL, 4M) and the reaction was stirred at 25° C. for 1 h. The mixture was concentrated to give (2,3-difluoro-4-(6-(1-methyl-1H-pyrazol-4-yl)pyrazolo[1,5-a]pyrazin-4-yl)phenyl)methanamine dihydrochloride (80 mg, crude) as a yellow solid. LCMS m/z=341.0 [M+H]⁺

### 3. Preparation of 2-(tert-butyl)-N-(2,3-difluoro-4-(6-(1-methyl-1H-pyrazol-4-yl)pyrazolo[1,5-a]pyrazin-4-yl)benzyl)-2H-tetrazole-5-carboxamide

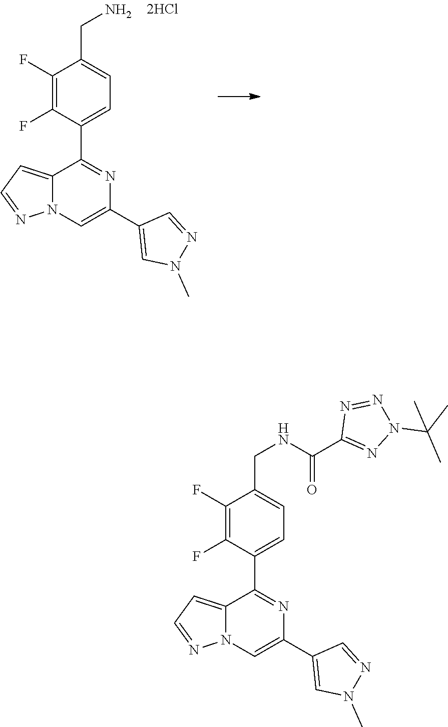 [SMILES: Cn1cc(-c2cn3nccc3c(-c3ccc(CN)c(F)c3F)n2)cn1 ; Cn1cc(-c2cn3nccc3c(-c3ccc(CNC(=O)c4nnn(C(C)(C)C)n4)c(F)c3F)n2)cn1]

To a solution of (2,3-difluoro-4-(6-(1-methyl-1H-pyrazol-4-yl)pyrazolo[1,5-a]pyrazin-4-yl)phenyl)methanamine dihydrochloride (80 mg, 235.1 μmol) in DCM (30 mL) was added DIPEA (60.76 mg, 470.1 μmol), 2-(tert-butyl)-2H-tetrazole-5-carboxylic acid (80 mg, 470.14 mol) and HATU (179.24 mg, 470.1 μmol) and the reaction was stirred at 25° C. for 1 h. The mixture was diluted with water (30 mL) and extracted with DCM (15 mL×3). The combined organic layers were concentrated under vacuum and the residue was purified by Prep-HPLC (Method D, 42-62% gradient) to give 2-(tert-butyl)-N-(2,3-difluoro-4-(6-(1-methyl-1H-pyrazol-4-yl)pyrazolo[1,5-a]pyrazin-4-yl)benzyl)-2H-tetrazole-5-carboxamide (37 mg, 32% yield) as a white solid. ¹H NMR (500 MHz, DMSO-d₆) δ: 9.71 (t, J=6.0 Hz, 1H), 9.24 (d, J=0.5 Hz, 1H), 8.28 (s, 1H), 8.18 (d, J=2.5 Hz, 1H), 8.08 (s, 1H), 7.62 (t, J=6.5 Hz, 1H), 7.40 (t, J=7.0 Hz, 1H), 6.87 (s, 1H), 4.67 (d, J=6.0 Hz, 2H), 3.88 (s, 3H), 1.74 (s, 9H).

### Example 88. 5-(tert-butyl)-N-(2,3-difluoro-4-(6-(1-methyl-1H-pyrazol-4-yl)pyrazolo[1,5-a]pyrazin-4-yl)benzyl)-1,2,4-oxadiazole-3-carboxamide

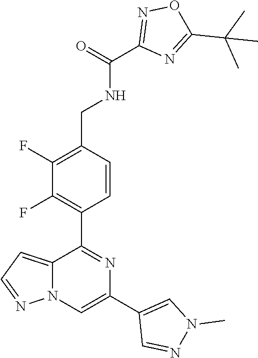 [SMILES: Cn1cc(-c2cn3nccc3c(-c3ccc(CNC(=O)c4noc(C(C)(C)C)n4)c(F)c3F)n2)cn1]

To a solution of (2,3-difluoro-4-(6-(1-methyl-1H-pyrazol-4-yl)pyrazolo[1,5-a]pyrazin-4-yl)phenyl)methanamine dihydrochloride (Example 87, step 2, 80 mg, 212.3 μmol) in DCM (30 mL) was added DIPEA (54.88 mg, 424.6 μmol) at 20° C. 5-(tert-Butyl)-1,2,4-oxadiazole-3-carboxylic acid (72.26 mg, 424.64 μmol) and HATU (97.13 mg, 254.78 μmol) were added and the reaction was stirred at 20° C. for 1 h. The mixture was poured into water (50 mL) and extracted with DCM (50 mL×3). The combined organic layers were washed with brine (100 mL), dried over Na₂SO₄, filtered and concentrated under vacuum. The crude product was purified by Prep-HPLC (Method D, 53-73% gradient) to give 5-(tert-butyl)-N-(2,3-difluoro-4-(6-(1-methyl-1H-pyrazol-4-yl)pyrazolo[1,5-a]pyrazin-4-yl)benzyl)-1,2,4-oxadiazole-3-carboxamide (43.8 mg, 41.9% yield) as a yellow solid. LCMS m/z=493.1 [M+H]⁺ 1H NMR (400 MHz, DMSO-d₆) δ: 9.66-9.62 (m, 1H), 9.24 (s, 1H), 8.29 (s, 1H), 8.18 (s, 1H), 8.08 (s, 1H), 7.65-7.61 (m, 1H), 7.41-7.31 (m, 1H), 6.87 (s, 1H), 4.64 (d, J=6.0 Hz, 2H), 3.89 (s, 3H), 1.43 (s, 9H).

### Examples 89 to 107

The compounds in the following table were prepared from the appropriate carboxylic acid and amine listed below, following a similar procedure to that described in Example 88.
Amine 1: (2,3-difluoro-4-(6-(1-methyl-1H-pyrazol-4-yl)pyrazolo[1,5-a]pyrazin-4-yl)phenyl)methanamine dihydrochloride (Example 87, step 2)
Amine 2: (2,5-difluoro-4-(6-(1-methyl-1H-pyrazol-4-yl)pyrazolo[1,5-a]pyrazin-4-yl)phenyl)methanamine dihydrochloride (Example 84, step 3)
Amine 3: (2-(difluoromethyl)-4-(6-(1-methyl-1H-pyrazol-4-yl)pyrazolo[1,5-a]pyrazin-4-yl)phenyl)methanamine dihydrochloride (Example 86, step 3)

| Example | Name/Structure/Starting materials (SM)/Data |
|---|---|
| 89 | 3-(tert-butyl)-N-(2,3-difluoro-4-(6-(1-methyl-1H-pyrazol-4- yl)pyrazolo[1,5-a]pyrazin-4-yl)benzyl)-1,2,4-oxadiazole-5-carboxamide 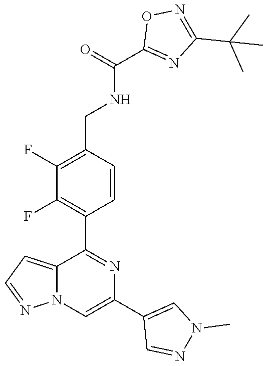 [SMILES: Cn1cc(-c2cn3nccc3c(-c3ccc(CNC(=O)c4nc(C(C)(C)C)no4)c(F)c3F)n2)cn1] SM: 3-(tert-butyl)-1,2,4-oxadiazole-5-carboxylic acid and Amine 1 Prep-HPLC (Method D, 52-72% gradient) 39.6 mg, 28.2% yield as a yellow solid. LCMS m/z = 493.1 [M + H]⁺. 1H NMR (400 MHz, DMSO-d₆) δ = 10.02 (t, J = 6.0 Hz, 1H), 9.25 (d, J = 0.8 Hz, 1H), 8.28 (s, 1H), 8.18 (d, J = 2.8 Hz, 1H), 8.08 (d, J = 0.8 Hz, 1H), 7.66-7.59 (m, 1H), 7.45 (t, J = 6.8 Hz, 1H), 6.87 (t, J = 2.4 Hz, 1H), 4.65 (d, J = 5.6 Hz, 2H), 3.89 (s, 3H), 1.37 (s, 9H) |
| 90 | 2-(tert-butyl)-N-(2,3-difluoro-4-(6-(1-methyl-1H-pyrazol-4- yl)pyrazolo[1,5-a]pyrazin-4-yl)benzyl)oxazole-4-carboxamide 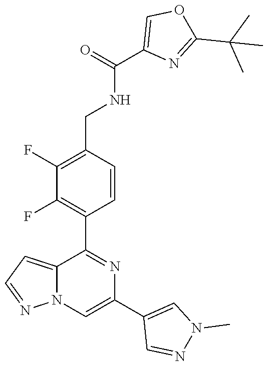 [SMILES: Cn1cc(-c2cn3nccc3c(-c3ccc(CNC(=O)c4coc(C(C)(C)C)n4)c(F)c3F)n2)cn1] SM: 2-(tert-butyl)oxazole-4-carboxylic acid and Amine 1 Prep-HPLC (Method D, 52-72% gradient) 55.6 mg, 52.4% yield as a yellow solid. LCMS m/z = 492.1 [M + H]⁺ 1H NMR: (400 MHz, DMSO-d₆) δ = 9.23 (d, J = 1.2 Hz, 1H), 8.81 (t, J = 6.4 Hz, 1H), 8.56-8.53 (m, 1H), 8.27 (s, 1H), 8.17 (d, J = 2.4 Hz, 1H), 8.07 (d, J = 0.8 Hz, 1H), 7.63-7.57 (m, 1H), 7.34 (t, J = 6.8 Hz, 1H), 6.86 (t, J = 2.4 Hz, 1H), 4.59 (d, J = 6.4 Hz, 2H), 3.88 (s, 3H), 1.36 (s, 9H) |
| 91 | 1-(tert-butyl)-N-(2,3-difluoro-4-(6-(1-methyl-1H-pyrazol-4- yl)pyrazolo[1,5-a]pyrazin-4-yl)benzyl)-1H-pyrazole-3-carboxamide 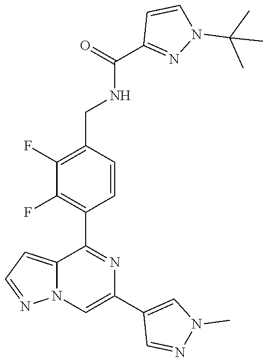 [SMILES: Cn1cc(-c2cn3nccc3c(-c3ccc(CNC(=O)c4ccn(C(C)(C)C)n4)c(F)c3F)n2)cn1] SM: 1-(tert-butyl)-1H-pyrazole-3-carboxylic acid and Amine 1 Prep-HPLC (Method D, 47-65% gradient) 68.0 mg, 48.6 % yield as a yellow solid. LCMS m/z = 491.0 [M + H]⁺ ¹H NMR (400 MHz, DMSO-d₆) δ = 9.24 (d, J = 0.8 Hz, 1H), 8.74 (t, J = 6.0 Hz, 1H), 8.29 (s, 1H), 8.18 (d, J = 2.8 Hz, 1H), 8.08 (s, 1H), 7.94 (d, J = 2.4 Hz, 1H), 7.65-7.59 (m, 1H), 7.36 (t, J = 6.4 Hz, 1H), 6.87 (t, J = 2.4 Hz, 1H), 6.67 (d, J = 2.4 Hz, 1H), 4.61 (d, J = 6.4 Hz, 2H), 3.89 (s, 3H), 1.59-1.55 (m, 9H) |
| 92 | 2-(tert-butyl)-N-(2,3-difluoro-4-(6-(1-methyl-1H-pyrazol-4- yl)pyrazolo[1,5-a]pyrazin-4-yl)benzyl)-2H-1,2,3-triazole-4-carboxamide 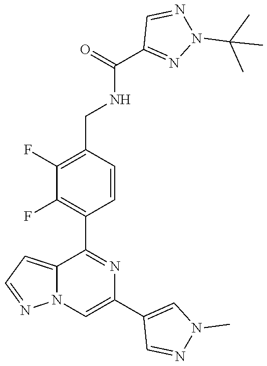 [SMILES: Cn1cc(-c2cn3nccc3c(-c3ccc(CNC(=O)c4cnn(C(C)(C)C)n4)c(F)c3F)n2)cn1] SM: 2-(tert-butyl)-2H-1,2,3-triazole-4-carboxylic acid and Amine 1 Prep-HPLC (Method D, 48-68% gradient) 87.0 mg, 60.2% yield as a white solid. LCMS m/z = 492.1 [M + H]⁺ ¹H NMR (400 MHz, DMSO-d₆) δ: 9.24 (d, J = 0.8 Hz, 1H), 9.10 (t, J = 6.0 Hz, 1H), 8.28 (s, 1H), 8.21-8.16 (m, 2H), 8.08 (s, 1H), 7.62 (t, J = 6.8 Hz, 1H), 7.38 (t, J = 6.4 Hz, 1H), 6.88-6.85 (m, 1H), 4.63 (d, J = 6.0 Hz, 2H), 3.88 (s, 3H), 1.65 (s, 9H). |
| 93 | 1-(tert-butyl)-N-(2,3-difluoro-4-(6-(1-methyl-1H-pyrazol-4- yl)pyrazolo[1,5-a]pyrazin-4-yl)benzyl)-1H-1,2,3-triazole-4-carboxamide 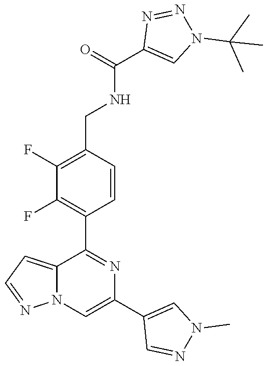 [SMILES: Cn1cc(-c2cn3nccc3c(-c3ccc(CNC(=O)c4cn(C(C)(C)C)nn4)c(F)c3F)n2)cn1] SM: 1-(tert-butyl)-1H-1,2,3-triazole-4-carboxylic acid and Amine 1 Prep-HPLC (Method E, 35-65% gradient) 40.0 mg, 34.6% yield as a yellow solid. LCMS m/z = 492.0 [M + H]⁺ ¹H NMR (400 MHz, DMSO-d₆) δ: 9.20 (s, 2H), 8.69 (s, 1H), 8.25 (s, 1H), 8.14 (d, J = 2.4 Hz, 1H), 8.04 (s, 1H), 7.57 (t, J = 6.8 Hz, 1H), 7.33 (t, J = 7.2 Hz, 1H), 6.83 (s, 1H), 4.61 (d, J = 5.6 Hz, 2H), 3.85 (s, 3H), 1.61 (s, 9H). |
| 94 | 3-(tert-butyl)-N-(2,5-difluoro-4-(6-(1-methyl-1H-pyrazol-4- yl)pyrazolo[1,5-a]pyrazin-4-yl)benzyl)-1,2,4-oxadiazole-5-carboxamide 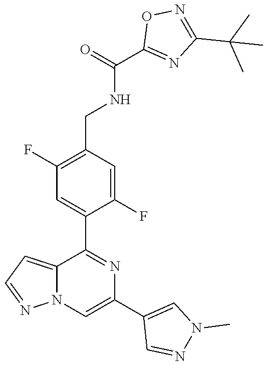 [SMILES: Cn1cc(-c2cn3nccc3c(-c3cc(F)c(CNC(=O)c4nc(C(C)(C)C)no4)cc3F)n2)cn1] SM: 3-tert-butyl-1,2,4-oxadiazole-5-carboxylic acid and Amine 2 Prep-HPLC (Method E, 41-71% gradient) 68.7 mg, 59.3% yield as a white solid. LCMS m/z = 493.1 [M + H]⁺ ¹H NMR: (400 MHz, DMSO-d₆) δ: 9.89 (t, J = 5.6 Hz, 1H), 9.21 (d, J = 0.8 Hz, 1H), 8.27 (s, 1H), 8.14 (d, J = 2.4 Hz, 1H), 8.05 (s, 1H), 7.67-7.62 (m, 1H), 7.53-7.48 (m, 1H), 6.79 (t, J = 2.0 Hz, 1H), 4.57 (d, J = 5.6 Hz, 2H), 3.86 (s, 3H), 1.34 (s, 9H). |
| 95 | 2-(tert-butyl)-N-(2,5-difluoro-4-(6-(1-methyl-1H-pyrazol-4- yl)pyrazolo[1,5-a]pyrazin-4-yl)benzyl)oxazole-4-carboxamide 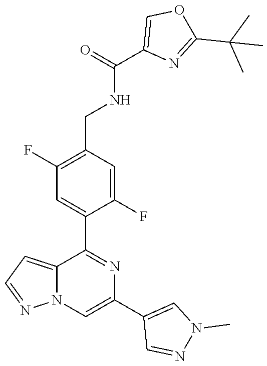 [SMILES: Cn1cc(-c2cn3nccc3c(-c3cc(F)c(CNC(=O)c4coc(C(C)(C)C)n4)cc3F)n2)cn1] SM: 2-(tert-butyl)oxazole-4-carboxylic acid and Amine 2 Prep-HPLC (Method D, 51-71% gradient) 49.2 mg, 47.2% yield as a yellow solid. LCMS m/z = 492.1 [M + H]⁺ ¹H NMR: (400 MHz, DMSO-d₆) δ = 9.23 (s, 1H), 8.59-8.54 (d, J = 6.0 Hz, 1H), 8.55 (d, J = 6.8 Hz, 1H), 8.30 (d, J = 6.0 Hz, 1H), 8.18-8.14 (m, 1H), 8.08 (d, J = 6.4 Hz, 1H), 7.68-7.62 (m, 1H), 7.39-7.34 (m, 1H), 6.82 (s, 1H), 4.57-4.55 (m, 2H), 3.88 (d, J = 7.2 Hz, 3H), 1.36 (d, J = 6.8 Hz, 9H). |
| 96 | 1-(tert-butyl)-N-(2,5-difluoro-4-(6-(1-methyl-1H-pyrazol-4- yl)pyrazolo[1,5-a]pyrazin-4-yl)benzyl)-1H-pyrazole-3-carboxamide 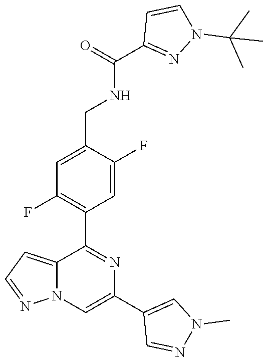 [SMILES: Cn1cc(-c2cn3nccc3c(-c3cc(F)c(CNC(=O)c4ccn(C(C)(C)C)n4)cc3F)n2)cn1] SM: 1-(tert-butyl)-1H-pyrazole-3-carboxylic acid and Amine 2 a yellow solid, 65 mg, 45.1%, LCMS m/z = 491.1 [M + H]⁺ ¹H NMR (400 MHz, DMSO-d₆) δ = 9.23 (d, J = 0.8 Hz, 1H), 8.71 (t, J = 6.0 Hz, 1H), 8.31 (s, 1H), 8.16 (d, J = 2.4 Hz, 1H), 8.09 (s, 1H), 7.94 (d, J = 2.4 Hz, 1H), 7.66 (dd, J = 5.6, 9.6 Hz, 1H), 7.36 (dd, J = 6.0, 10.4 Hz, 1H), 6.83 (t, J = 2.4 Hz, 1H), 6.68 (d, J = 2.4 Hz, 1H), 4.57 (d, J = 6.0 Hz, 2H), 3.89 (s, 3H), 1.57 (s, 9H). |
| 97 | 2-(tert-butyl)-N-(2,5-difluoro-4-(6-(1-methyl-1H-pyrazol-4- yl)pyrazolo[1,5-a]pyrazin-4-yl)benzyl)-2H-1,2,3-triazole-4-carboxamide 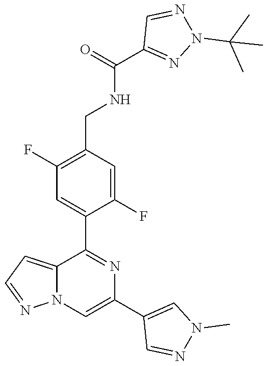 [SMILES: Cn1cc(-c2cn3nccc3c(-c3cc(F)c(CNC(=O)c4cnn(C(C)(C)C)n4)cc3F)n2)cn1] SM: 2-(tert-butyl)-2H-1,2,3-triazole-4-carboxylic acid and Amine 2 Prep-HPLC (Method D, 46-66% gradient). 41.10 mg, 39.4% yield as a yellow solid. LCMS m/z = 492.1 [M + H]⁺ 1H NMR: (500 MHz, DMSO-d₆) δ ppm 9.26-9.25 (m, 1H), 9.08 (t, J = 6.0 Hz, 1H), 8.32 (s, 1H), 8.22-8.17 (m, 2H), 8.13-8.10 (m, 1H), 7.70-7.66 (m, 1H), 7.44-7.40 (m, 1H), 6.85-6.82 (m, 1H), 4.60 (d, J = 7.0 Hz, 2H), 3.91 (s, 3H), 1.67 (s, 9H). |
| 98 | 1-(tert-butyl)-N-(2,5-difluoro-4-(6-(1-methyl-1H-pyrazol-4- yl)pyrazolo[1,5-a]pyrazin-4-yl)benzyl)-1H-1,2,3-triazole-4-carboxamide 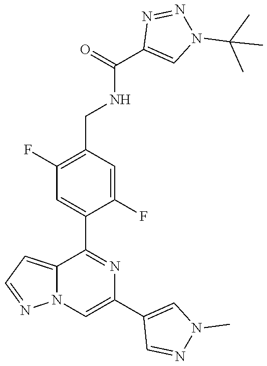 [SMILES: Cn1cc(-c2cn3nccc3c(-c3cc(F)c(CNC(=O)c4cn(C(C)(C)C)nn4)cc3F)n2)cn1] SM: 1-(tert-butyl)-1H-1,2,3-triazole-4-carboxylic acid and Amine 2 Prep-HPLC (Method D, 39-59% gradient) 62.9 mg, 48.4% yield as a yellow solid. LCMS m/z = 492.1 [M + H]⁺ 1HNMR (500 MHz, DMSO-d₆) δ: 9.26-9.22 (m, 2H), 8.75 (s, 1H), 8.31 (s, 1H), 8.17 (d, J = 2.0 Hz, 1H), 8.10 (s, 1H), 7.69-7.66 (m, 1H), 7.40-7.36 (m, 1H), 6.85-6.84 (m, 1H), 4.60 (d, J = 6.5 Hz, 2H), 3.91 (s, 3H), 1.66 (s, 9H). |
| 99 | 2-(tert-butyl)-N-(2,5-difluoro-4-(6-(1-methyl-1H-pyrazol-4- yl)pyrazolo[1,5-a]pyrazin-4-yl)benzyl)-2H-tetrazole-5-carboxamide 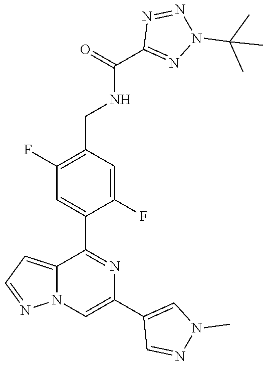 [SMILES: Cn1cc(-c2cn3nccc3c(-c3cc(F)c(CNC(=O)c4nnn(C(C)(C)C)n4)cc3F)n2)cn1] SM: 2-(tert-butyl)-2H-tetrazole-5-carboxylic acid and Amine 2 Prep-HPLC (Method D, 42-62% gradient) 61.20 mg, 52.0% yield as a white solid. LCMS m/z = 493.2 [M + H]⁺ ¹H NMR: (500 MHz, DMSO-d₆) δ ppm = 9.68 (t, J = 6.0 Hz, 1H), 9.24 (d, J = 1.0 Hz, 1H), 8.32 (s, 1H), 8.18 (d, J = 2.5 Hz, 1H), 8.10 (s, 1H), 7.71- 7.67 (m, 1H),7.48-7.44 (m, 1H), 6.84-6.83 (m, 1H), 4.64 (d, J = 6.0 Hz, 2H), 3.91 (s, 3H), 1.76 (s, 9H). |
| 100 | 3-(tert-butyl)-N-(2-(difluoromethyl)-4-(6-(1-methyl-1H-pyrazol-4- yl)pyrazolo[1,5-a]pyrazin-4-yl)benzyl)-1,2,4-oxadiazole-5-carboxamide 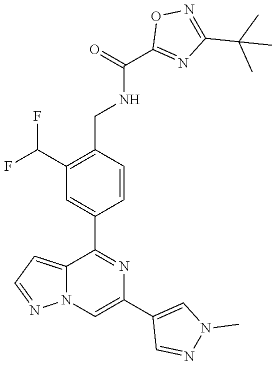 [SMILES: Cn1cc(-c2cn3nccc3c(-c3ccc(CNC(=O)c4nc(C(C)(C)C)no4)c(C(F)F)c3)n2)cn1] SM: 3-(tert-butyl)-1,2,4-oxadiazole-5-carboxylic acid and Amine 3 Prep-HPLC (Method D, 52-72% gradient) 50.1 mg, 38.9% yield as a yellow solid. LCMS m/z = 5-7.1 [M + H]⁺ ¹H NMR (500 MHz, DMSO-d₆) δ: 10.02 (t, J = 6.0 Hz, 1H), 9.20-9.19 (m, 1H), 8.34-8.31 (m, 3H), 8.23 (d, J = 2.5 Hz, 1H), 8.14-8.13 (m, 1H), 7.72 (d, J = 7.5 Hz, 1H), 7.60-7.37 (m, 1H), 7.18-7.16 (m, 1H), 4.76 (d, J = 6.0 Hz, 2H), 3.93 (s, 3H), 1.39 (s, 9H). |
| 101 | 2-(tert-butyl)-N-(2-(difluoromethyl)-4-(6-(1-methyl-1H-pyrazol-4- yl)pyrazolo[1,5-a]pyrazin-4-yl)benzyl)oxazole-4-carboxamide 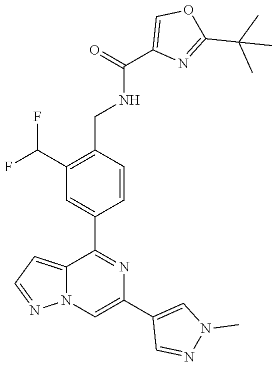 [SMILES: Cn1cc(-c2cn3nccc3c(-c3ccc(CNC(=O)c4coc(C(C)(C)C)n4)c(C(F)F)c3)n2)cn1] SM: 2-(tert-butyl)-oxazole-4-carboxylic acid and Amine 3 Prep-HPLC (Method D, 55-75% gradient) 40.0 mg, 36.1% yield as a pale white solid. LCMS m/z = 506.1 [M + H]⁺ ¹H NMR (400 MHz, DMSO-d₆) δ: 9.15 (s, 1H), 8.79 (t, J = 6.4 Hz, 1H), 8.54 (s, 1H), 8.30-8.25 (m, 3H), 8.18 (d, J = 2.4 Hz, 1H), 8.09 (s, 1H), 7.64-7.34 (m, 2H), 7.13-7.12 (m, 1H), 4.66 (d, J = 6.0 Hz, 2H), 3.88 (s, 3H), 1.34 (s, 9H). |
| 102 | 5-(tert-butyl)-N-(2-(difluoromethyl)-4-(6-(1-methyl-1H-pyrazol-4- yl)pyrazolo[1,5-a]pyrazin-4-yl)benzyl)-1,3,4-oxadiazole-2-carboxamide 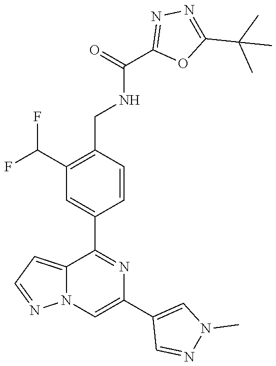 [SMILES: Cn1cc(-c2cn3nccc3c(-c3ccc(CNC(=O)c4nnc(C(C)(C)C)o4)c(C(F)F)c3)n2)cn1] SM: 5-(tert-butyl)-1,3,4-oxadiazole-2-carboxylic acid and Amine 3 as a yellow solid, 53 mg, 37.1%, Prep-HPLC (Method F, 44-66% gradient) LCMS m/z = 507.1 [M + H]⁺ ¹H NMR: (400 MHz, DMSO-d₆) δ: 9.95 (t, J = 6.0 Hz, 1H), 9.19 (d, J = 0.8 Hz, 1H), 8.33 (s, 2H), 8.30 (d, J = 8.0 Hz, 1H), 8.22 (d, J = 2.4 Hz, 1H), 8.12 (d, J = 0.8 Hz, 1H), 7.70 (d, J = 8.0 Hz, 1H), 7.63-7.33 (m, 1H), 7.16 (dd, J = 0.8, 2.4 Hz, 1H), 4.74 (d, J = 5.6 Hz, 2H), 3.91 (s, 3H), 1.41 (s, 9H). |
| 103 | 1-(tert-butyl)-N-(2-(difluoromethyl)-4-(6-(1-methyl-1H-pyrazol-4- yl)pyrazolo[1,5-a]pyrazin-4-yl)benzyl)-1H-pyrazole-3-carboxamide 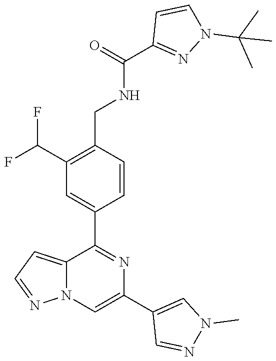 [SMILES: Cn1cc(-c2cn3nccc3c(-c3ccc(CNC(=O)c4ccn(C(C)(C)C)n4)c(C(F)F)c3)n2)cn1] SM: 1-(tert-butyl)-1H-pyrazole-3-carboxylic acid and Amine 3 Prep-HPLC (Method D, 49-69% gradient) 50.0 mg, 45.2% yield as a pale white solid. LCMS m/z = 505.2 [M + H]⁺ 1H NMR (400 MHz, DMSO-d₆) δ: 9.15 (s, 1H), 8.70 (t, J = 6.0 Hz, 1H), 8.31-8.25 (m, 3H), 8.18 (d, J = 2.4 Hz, 1H), 8.09 (s, 1H), 7.91 (d, J = 2.4 Hz, 1H), 7.65-7.35 (m, 2H), 7.13 (d, J = 2.0 Hz, 1H), 6.65 (d, J = 2.0 Hz, 1H), 4.67 (d, J = 6.0 Hz, 2H), 3.88 (s, 3H), 1.54 (s, 9H). |
| 104 | 2-(tert-butyl)-N-(2-(difluoromethyl)-4-(6-(1-methyl-1H-pyrazol-4- yl)pyrazolo[1,5-a]pyrazin-4-yl)benzyl)-2H-1,2,3-triazole-4-carboxamide 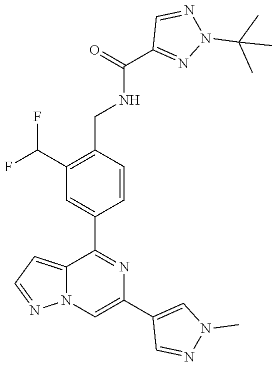 [SMILES: Cn1cc(-c2cn3nccc3c(-c3ccc(CNC(=O)c4cnn(C(C)(C)C)n4)c(C(F)F)c3)n2)cn1] SM: 2-(tert-butyl)-2H-1,2,3-triazole-4-carboxylic acid and Amine 3 Prep-HPLC (Method D, 51-71% gradient) 32.0 mg, 41.2% yield as a yellow solid. LCMS m/z = 506.3 [M + H]⁺ ¹H NMR: (400 MHz, DMSO-d₆) δ = 9.14 (s, 1H), 9.08 (t, J = 6.0 Hz, 1H), 8.28-8.26 (m, 3H), 8.19-8.15 (m, 2H), 8.08 (s, 1H), 7.64-7.61 (m, 1H), 7.48-7.30 (m, 1H), 7.13-7.10 (m, 1H), 4.70-4.68 (m, 2H), 3.88 (s, 3H), 1.62 (s, 9H) |
| 105 | 1-(tert-butyl)-N-(2-(difluoromethyl)-4-(6-(1-methyl-1H-pyrazol-4- yl)pyrazolo[1,5-a]pyrazin-4-yl)benzyl)-1H-1,2,3-triazole-4-carboxamide 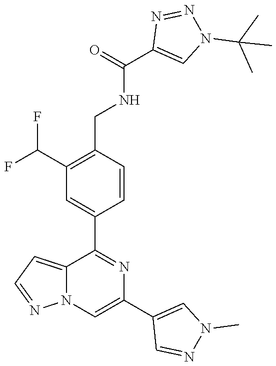 [SMILES: Cn1cc(-c2cn3nccc3c(-c3ccc(CNC(=O)c4cn(C(C)(C)C)nn4)c(C(F)F)c3)n2)cn1] SM: 1-(tert-butyl)-1H-1,2,3-triazole-4-carboxylic acid and Amine 3 Prep-HPLC (Method D, 44-64% gradient) 59.6 mg, 51.2% yield as a white solid. LCMS m/z = 506.1 [M + H]⁺ ¹H NMR (400 MHz, DMSO-d₆) δ: 9.24-9.20 (m, 1H), 9.14 (s, 1H), 8.70 (s, 1H), 8.30-8.24 (m, 3H), 8.18 (d, J = 2.4 Hz, 1H), 8.09 (s, 1H), 7.64-7.35 (m, 2H), 7.12 (d, J = 2.0 Hz, 1H), 4.70 (d, J = 6.0 Hz, 2H), 3.88 (s, 3H), 1.61 (s, 9H). |
| 106 | 2-(tert-butyl)-N-(2-(difluoromethyl)-4-(6-(1-methyl-1H-pyrazol-4- yl)pyrazolo[1,5-a]pyrazin-4-yl)benzyl)-2H-tetrazole-5-carboxamide 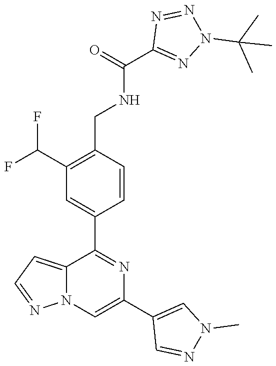 [SMILES: Cn1cc(-c2cn3nccc3c(-c3ccc(CNC(=O)c4nnn(C(C)(C)C)n4)c(C(F)F)c3)n2)cn1] SM: 2-(tert-butyl)-2H-tetrazole-5-carboxylic acid and Amine 3 Prep-HPLC (Method D, 47-67% gradient) 33 mg, 50.9% yield as a yellow solid. LCMS m/z = 507.3 [M + H]⁺ 1H NMR (400 MHz, DMSO-d₆) δ: 9.69 (t, J = 6.0 Hz, 1H), 9.15 (s, 1H), 8.31-8.25 (m, 3H), 8.18 (d, J = 2.4 Hz, 1H), 8.09 (s, 1H), 7.64 (d, J = 7.8 Hz, 1H), 7.62-7.34 (m, 1H), 7.13-7.08 (m, 1H), 4.74 (d, J = 6.0 Hz, 2H), 3.88 (s, 3H), 1.71 (s, 9H) |
| 107 | 1-(tert-butyl)-N-(2-(difluoromethyl)-4-(6-(1-methyl-1H-pyrazol-4- yl)pyrazolo[1,5-a]pyrazin-4-yl)benzyl)-1H-pyrazole-4-carboxamide 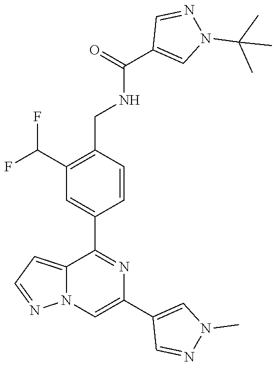 [SMILES: Cn1cc(-c2cn3nccc3c(-c3ccc(CNC(=O)c4cnn(C(C)(C)C)c4)c(C(F)F)c3)n2)cn1] SM: 1-(tert-butyl)-1H-pyrazole-4-carboxylic acid and Amine 3 Prep-HPLC (Method G, 35-65% gradient) 47.9 mg, 42.1% yield as a white solid. LCMS m/z = 505.0 [M + H]⁺ 1H NMR: (500 MHz, DMSO-d₆) δ: 9.19 (d, J = 0.5 Hz, 1H), 8.74 (t, J = 6.0 Hz, 1H), 8.38-8.30 (m, 4H), 8.23 (d, J = 2.5 Hz, 1H), 8.14 (s, 1H), 7.96 (s, 1H), 7.66 (d, J = 8.0 Hz, 1H), 7.61-7.36 (m, 1H), 7.18-7.16 (m, 1H), 4.70 (d, J = 6.0 Hz, 2H), 3.93 (s, 3H), 1.55 (s, 9H). |

### Example 108. 3-(tert-butyl)-N-(3-fluoro-4-(6-(1-methyl-1H-pyrazol-4-yl)pyrazolo[1,5-a]pyrazin-4-yl)benzyl)-1,2,4-oxadiazole-5-carboxamide

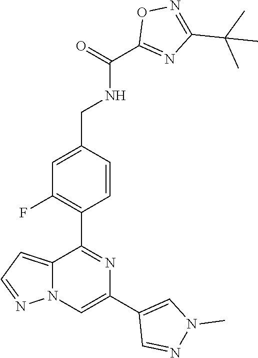 [SMILES: Cn1cc(-c2cn3nccc3c(-c3ccc(CNC(=O)c4nc(C(C)(C)C)no4)cc3F)n2)cn1]

To a solution of (3-fluoro-4-(6-(1-methyl-1H-pyrazol-4-yl)pyrazolo[1,5-a]pyrazin-4-yl)phenyl)methanamine dihydrochloride (Example 83, step 3, 80 mg, 223 μmol) in DCM (50 mL) was added DIPEA (86.45 mg, 669 μmol), 3-(tert-butyl)-1,2,4-oxadiazole-5-carboxylic acid (56.91 mg, 334.5 μmol) and HATU (85 mg, 223 μmol) and the mixture was stirred at 20° C. for 2 h. The mixture was concentrated under vacuum and the crude was purified by Prep-HPLC (Method D, 55-75% gradient) to give 3-(tert-butyl)-N-(3-fluoro-4-(6-(1-methyl-1H-pyrazol-4-yl)pyrazolo[1,5-a]pyrazin-4-yl)benzyl)-1,2,4-oxadiazole-5-carboxamide (41.8 mg, 39.5% yield) as a yellow solid. LCMS m/z=475.1 [M+H]⁺ 1H NMR (400 MHz, DMSO-d₆) δ: 9.97 (t, J=4.0 Hz, 1H), 9.21 (s, 1H), 8.28 (s, 1H), 8.15 (s, 1H), 8.07 (s, 1H), 7.78 (t, J=8.0 Hz, 1H), 7.44-7.38 (m, 2H), 6.77 (s, 1H), 4.58 (d, J=4.0 Hz, 2H), 3.88 (s, 3H), 1.37 (s, 9H).

### Examples 109 to 114

The compounds in the following table were prepared from (3-fluoro-4-(6-(1-methyl-1H-pyrazol-4-yl)pyrazolo[1,5-a]pyrazin-4-yl)phenyl)methanamine dihydrochloride (Example 83, step 3) and appropriate heterocyclic carboxylic acid, following a similar procedure to that described in Example 108.

| Example No | Name/Structure/Starting Materials (SM)/Data |
|---|---|
| 109 | 2-(tert-butyl)-N-(3-fluoro-4-(6-(1-methyl-1H-pyrazol-4-yl)pyrazolo[1,5- alpyrazin-4-yl)benzyl)oxazole-4-carboxamide 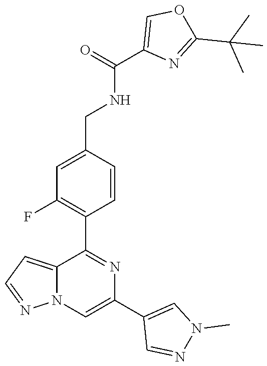 [SMILES: Cn1cc(-c2cn3nccc3c(-c3ccc(CNC(=O)c4coc(C(C)(C)C)n4)cc3F)n2)cn1] SM: 2-(tert-butyl)oxazole-4-carboxylic acid Prep-HPLC (Method D, 49-66% gradient) 44 mg, 41.7% yield, as a yellow solid. LCMS m/z = 474.1 [M + H]⁺ 1H NMR (400 MHz, DMSO-d₆) δ: 9.19 (s, 1H), 8.82 (t, J = 4.0 Hz, 1H), 8.54 (s, 1H), 8.28 (s, 1H), 8.15 (s, 1H), 8.07 (s, 1H), 7.76 (t, J = 8.0 Hz, 1H), 7.35 (d, J = 8.0 Hz, 2H), 6.77 (s, 1H), 4.53 (d, J = 8.0 Hz, 2H), 3.88 (s, 3H), 1.36 (s, 9H). |
| 110 | 4-(tert-butyl)-N-(3-fluoro-4-(6-(1-methyl-1H-pyrazol-4-yl)pyrazolo[1,5- a]pyrazin-4-yl)benzyl)oxazole-2-carboxamide 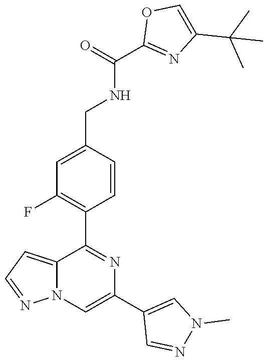 [SMILES: Cn1cc(-c2cn3nccc3c(-c3ccc(CNC(=O)c4nc(C(C)(C)C)co4)cc3F)n2)cn1] SM: 4-(tert-butyl)oxazole-2-carboxylic acid 60 mg, 51.0% yield as a white solid. LCMS m/z = 474.1 [M + H]⁺ ¹H NMR (500 MHz, DMSO-d₆) δ: 9.48 (t, J = 5.0 Hz, 1H), 9.20 (s, 1H), 8.28 (s, 1H), 8.14 (s, 1H), 8.06 (d, J = 10.0 Hz, 1H), 7.77 (t, J = 5.0 Hz, 1H), 7.37 (t, J = 10.0 Hz, 2H), 6.78 (s, 1H), 4.54 (s, 2H), 3.88 (s, 3H), 1.26 (s, 9H). |
| 111 | 1-(tert-butyl)-N-(3-fluoro-4-(6-(1-methyl-1H-pyrazol-4-yl)pyrazolo[1,5- a]pyrazin-4-yl)benzyl)-1H-pyrazole-3-carboxamide 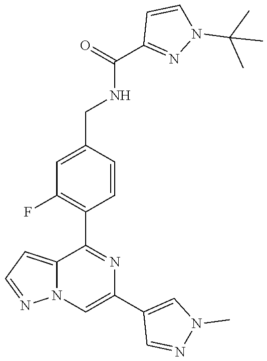 [SMILES: Cn1cc(-c2cn3nccc3c(-c3ccc(CNC(=O)c4ccn(C(C)(C)C)n4)cc3F)n2)cn1] SM: 1-(tert-butyl)-1H-pyrazole-3-carboxylic acid 39 mg, 33.3% yield as an orange solid. LCMS m/z = 473.0 [M + H]⁺ ¹H NMR (400 MHz, DMSO-d₆) δ: 9.20 (s, 1H), 8.74 (s, 1H), 8.29 (s, 1H), 8.15 (s, 1H), 8.08 (s, 1H), 7.94 (s, 1H), 7.76 (d, J = 8.0 Hz, 1H), 7.36 (t, J = 4.0 Hz, 2H), 6.79 (s, 1H), 6.67 (s, 1H), 4.55 (d, J = 8.0 Hz, 2H), 3.89 (s, 3H), 1.57 (s, 9H). |
| 112 | 1-(tert-butyl)-N-(3-fluoro-4-(6-(1-methyl-1H-pyrazol-4-yl)pyrazolo[1,5- a]pyrazin-4-yl)benzyl)-1H-1,2,3-triazole-4-carboxamide 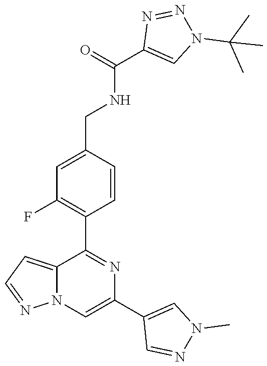 [SMILES: Cn1cc(-c2cn3nccc3c(-c3ccc(CNC(=O)c4cn(C(C)(C)C)nn4)cc3F)n2)cn1] SM: 1-(tert-butyl)-1H-1,2,3-triazole-4-carboxylic acid 51 mg, 42.8% yield as a white solid. Prep-HPLC (Method D, 42-62% gradient) LCMS m/z = 474.1 [M + H]⁺ ¹H NMR (400 MHz, DMSO-d₆) δ: 9.23 (t, J = 4.0 Hz, 1H), 9.19 (s, 1H), 8.71 (s, 1H), 8.27 (s, 1H), 8.14 (s, 1H), 8.06 (s, 1H), 7.76 (t, J = 8.0 Hz, 1H), 7.35 (t, J = 4.0 Hz, 2H), 6.77 (s, 1H), 4.57 (d, J = 8.0 Hz, 2H), 3.88 (s, 3H), 1.64 (s, 9H). |
| 113 | 2-(tert-butyl)-N-(3-fluoro-4-(6-(1-methyl-1H-pyrazol-4-yl)pyrazolo[1,5- a]pyrazin-4-yl)benzyl)-2H-1,2,3-triazole-4-carboxamide 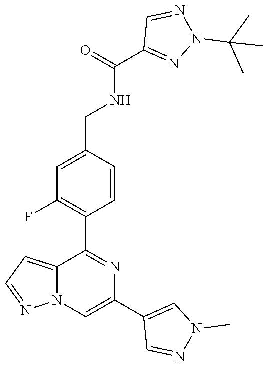 [SMILES: Cn1cc(-c2cn3nccc3c(-c3ccc(CNC(=O)c4cnn(C(C)(C)C)n4)cc3F)n2)cn1] SM: 2-(tert-butyl)-2H-1,2,3-triazole-4-carboxylic acid Prep-HPLC (Method D, 47-67% gradient) 56 mg, 59.0% yield as a yellow solid. LCMS m/z = 474.1 [M + H]⁺ 1H NMR (400 MHz, DMSO-d₆) δ: 9.20 (s, 1H), 9.08 (t, J = 4.0 Hz, 1H), 8.27 (s, 1H), 8.17-8.14 (m, 2H), 8.07 (s, 1H), 7.77 (t, J = 8.0 Hz, 1H), 7.37 (d, J = 8.0 Hz, 2H), 6.77 (s, 1H), 4.56 (d, J = 4.0 Hz, 2H), 3.88 (s, 3H), 1.65 (s, 9H). |
| 114 | 2-(tert-butyl)-N-(3-fluoro-4-(6-(1-methyl-1H-pyrazol-4-yl)pyrazolo[1,5- a]pyrazin-4-yl)benzyl)-2H-tetrazole-5-carboxamide 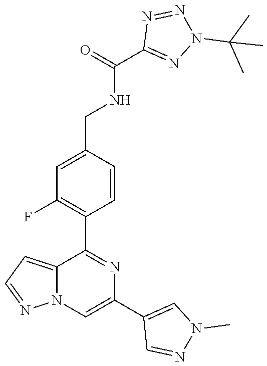 [SMILES: Cn1cc(-c2cn3nccc3c(-c3ccc(CNC(=O)c4nnn(C(C)(C)C)n4)cc3F)n2)cn1] SM: 2-(tert-butyl)-2H-tetrazole-5-carboxylic acid Prep-HPLC (Method D, 41-61% gradient) 60 mg, 69.3% yield as a yellow solid. LCMS m/z = 475.1 [M + H]⁺ 1HNMR (400 MHz, DMSO-d₆) δ: 9.70 (t, J = 6.4 Hz, 1H), 9.20 (s, 1H), 8.28 (s, 1H), 8.15 (d, J = 2.4 Hz, 1H), 8.07 (s, 1H), 7.78 (t, J = 8.0 Hz, 1H), 7.40-7.37 (m, 2H), 6.77 (s, 1H), 4.60 (d, J = 6.0 Hz, 2H), 3.88 (s, 3H), 1.74 (s, 9H). |

### Example 115: 2-(tert-butyl)-N-((5-(6-(1-methyl-1H-pyrazol-4-yl)pyrazolo[1,5-a]pyrazin-4-yl)pyridin-2-yl)methyl)-2H-tetrazole-5-carboxamide

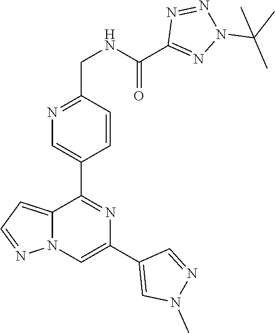 [SMILES: Cn1cc(-c2cn3nccc3c(-c3ccc(CNC(=O)c4nnn(C(C)(C)C)n4)nc3)n2)cn1]

### 1. Preparation of tert-butyl ((5-(6-(1-methyl-1H-pyrazol-4-yl)pyrazolo[1,5-a]pyrazin-4-yl)pyridin-2-yl)methyl)carbamate

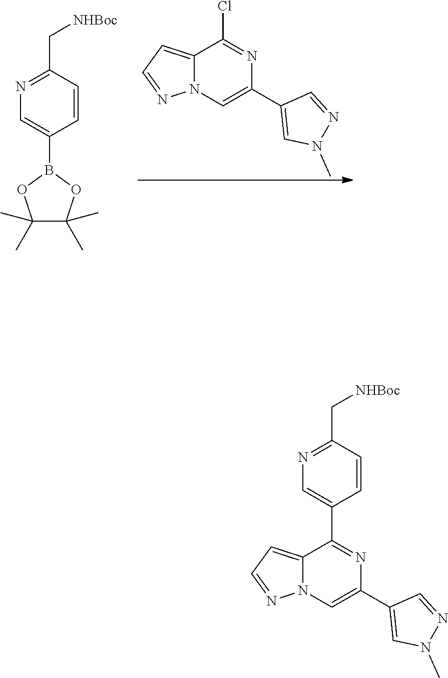

tert-Butyl ((5-(6-(1-methyl-1H-pyrazol-4-yl)pyrazolo[1,5-a]pyrazin-4-yl)pyridin-2-yl)methyl)carbamate was obtained, 85 mg, 77% yield as a light yellow solid, from tert-butyl ((5-(4,4,5,5-tetramethyl-1,3,2-dioxaborolan-2-yl)pyridin-2-yl)methyl)carbamate and 4-chloro-6-(1-methyl-1H-pyrazol-4-yl)pyrazolo[1,5-a]pyrazine (Example 1, step 5), following the procedure described in Example 79, step 6. LCMS m/z=406.2 [M+H]⁺

### 2. Preparation of (5-(6-(1-methyl-1H-pyrazol-4-yl)pyrazolo[1,5-a]pyrazin-4-yl)pyridin-2-yl)methanamine hydrochloride

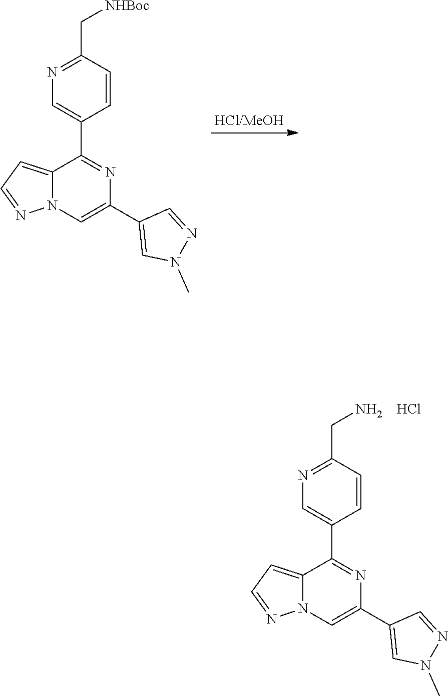 [SMILES: Cn1cc(-c2cn3nccc3c(-c3ccc(CNC(=O)OC(C)(C)C)nc3)n2)cn1 ; Cn1cc(-c2cn3nccc3c(-c3ccc(CN)nc3)n2)cn1 ; Cl]

tert-Butyl ((5-(6-(1-methyl-1H-pyrazol-4-yl)pyrazolo[1,5-a]pyrazin-4-yl)pyridin-2-yl)methyl)carbamate (85 mg, 209.6 μmol) was dissolved in MeOH (2.10 mL), HCl solution (1.25M in MeOH, 1.68 mL) was added and the reaction was stirred overnight at 50° C. The reaction was concentrated and vacuum dried to afford (5-(6-(1-methyl-1H-pyrazol-4-yl)pyrazolo[1,5-a]pyrazin-4-yl)pyridin-2-yl)methanamine hydrochloride (75 mg, crude) as a yellow solid. LCMS m/z=306.1 [M+H]⁺

### 3. Preparation of 2-(tert-butyl)-N-((5-(6-(1-methyl-1H-pyrazol-4-yl)pyrazolo[1,5-a]pyrazin-4-yl)pyridin-2-yl)methyl)-2H-tetrazole-5-carboxamide

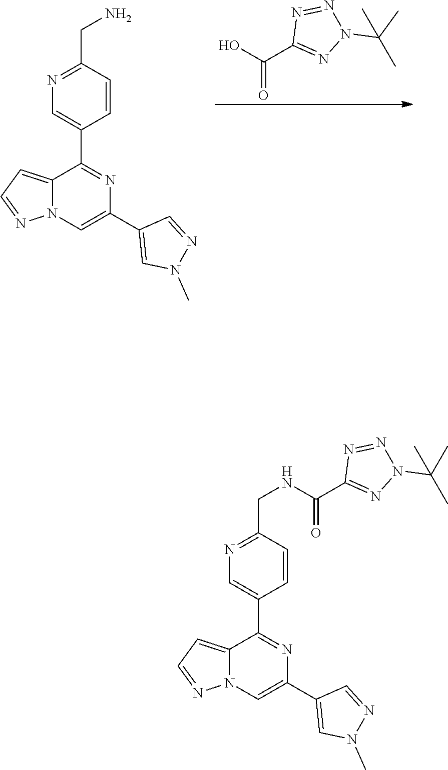 [SMILES: Cn1cc(-c2cn3nccc3c(-c3ccc(CN)nc3)n2)cn1 ; CC(C)(C)n1nnc(C(=O)O)n1 ; Cn1cc(-c2cn3nccc3c(-c3ccc(CNC(=O)c4nnn(C(C)(C)C)n4)nc3)n2)cn1]

A vial was charged with (5-(6-(1-methyl-1H-pyrazol-4-yl)pyrazolo[1,5-a]pyrazin-4-yl)pyridin-2-yl)methanamine hydrochloride (35 mg, 102.4 μmol), 2-(tert-butyl)-2H-tetrazole-5-carboxylic acid (20.91 mg, 122.9 μmol) and DCM (1.02 mL), followed by DIPEA (132.34 mg, 1.02 mmol) and the solution cooled to 0° C. HATU (46.85 mg, 122.9 μmol) was added in a single portion and the reaction was stirred overnight at rt. The reaction was concentrated and purified via column chromatography (gradient elution 0-100% [3:1 EtOAc:EtOH]:Heptane) to afford 2-(tert-butyl)-N-((5-(6-(1-methyl-1H-pyrazol-4-yl)pyrazolo[1,5-a]pyrazin-4-yl)pyridin-2-yl)methyl)-2H-tetrazole-5-carboxamide (40 mg, 84.5% yield) as a yellow solid. LCMS m/z=458.2 [M+H]^{+ 1}H NMR (500 MHz, DMSO-d₆) δ ppm 9.66 (t, J=6.10 Hz, 1H) 9.27 (d, J=2.44 Hz, 1H) 9.19 (s, 1H) 8.52 (dd, J=7.94, 2.44 Hz, 1H) 8.37 (s, 1H) 8.22 (d, J=2.44 Hz, 1H) 8.13 (s, 1H) 7.58 (d, J=7.94 Hz, 1H) 7.24 (dd, J=2.44, 1.22 Hz, 1H) 4.73 (d, J=6.10 Hz, 2H) 3.90 (s, 3H) 1.75 (s, 9H).

### Example 116: 5-(tert-butyl)-N-((5-(6-(1-methyl-1H-pyrazol-4-yl)pyrazolo[1,5-a]pyrazin-4-yl)pyridin-2-yl)methyl)-1,2,4-oxadiazole-3-carboxamide

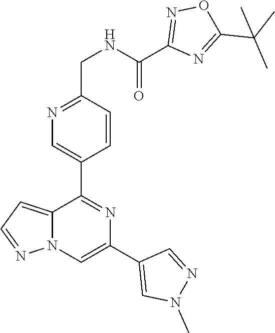 [SMILES: Cn1cc(-c2cn3nccc3c(-c3ccc(CNC(=O)c4noc(C(C)(C)C)n4)nc3)n2)cn1]

5-(tert-Butyl)-N-((5-(6-(1-methyl-1H-pyrazol-4-yl)pyrazolo[1,5-a]pyrazin-4-yl)pyridin-2-yl)methyl)-1,2,4-oxadiazole-3-carboxamide was obtained as a yellow solid, 45 mg, 74.3% yield, from (5-(6-(1-methyl-1H-pyrazol-4-yl)pyrazolo[1,5-a]pyrazin-4-yl)pyridin-2-yl)methanamine hydrochloride and 5-tert-butyl-1,2,4-oxadiazole-3-carboxylic acid, following the method described in Example 115, step 3. LCMS m/z=458.2 [M+H]^{+ 1}H NMR (500 MHz, DMSO-d₆) δ ppm 9.58 (t, J=6.10 Hz, 1H) 9.27 (d, J=1.83 Hz, 1H) 9.20 (s, 1H) 8.52 (dd, J=8.24, 2.14 Hz, 1H) 8.37 (s, 1H) 8.22 (d, J=2.44 Hz, 1H) 8.13 (s, 1H) 7.57 (d, J=7.94 Hz, 1H) 7.27-7.20 (m, 1H) 4.70 (d, J=6.10 Hz, 2H) 3.91 (s, 3H) 1.45 (s, 9H).

### Example 117: 5-(tert-butyl)-N-(2-fluoro-4-(6-(1-methyl-1H-pyrazol-4-yl)pyrazolo[1,5-a]pyrazin-4-yl)benzyl)-1,2,4-oxadiazole-3-carboxamide

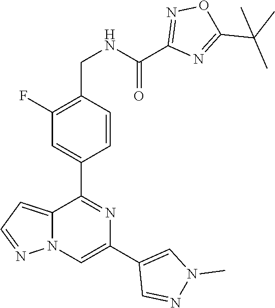 [SMILES: Cn1cc(-c2cn3nccc3c(-c3ccc(CNC(=O)c4noc(C(C)(C)C)n4)c(F)c3)n2)cn1]

DABAL-Me₃ (47.71 mg, 186.1 μmol) was added to a mixture of (2-fluoro-4-(6-(1-methyl-1H-pyrazol-4-yl)pyrazolo[1,5-a]pyrazin-4-yl)phenyl)methanamine dihydrochloride (Example 6, step 2, 40.0 mg, 124.1 μmol) and ethyl 5-tert-butyl-1,2,4-oxadiazole-3-carboxylate (36.90 mg, 186.14 μmol) in THF (1.24 mL) and the reaction was heated at 45° C. overnight. The mixture was diluted with MeOH (formed a thick gel) and filtered. The filtrate was concentrated and purified by column chromatography (gradient elution 0-75% [EtOAc:EtOH]:heptane) to afford 5-(tert-butyl)-N-(2-fluoro-4-(6-(1-methyl-1H-pyrazol-4-yl)pyrazolo[1,5-a]pyrazin-4-yl)benzyl)-1,2,4-oxadiazole-3-carboxamide (36.0 mg, 58.1% yield) as a light yellow solid. LCMS m/z=475.2 [M+H]⁺ 1H NMR (500 MHz, DMSO-d₆) δ ppm 9.59 (t, J=6.10 Hz, 1H) 9.18 (d, J=1.22 Hz, 1H) 8.37 (s, 1H) 8.21 (d, J=2.44 Hz, 1H) 8.13 (s, 1H) 8.00 (dd, J=7.94, 1.83 Hz, 1H) 7.94 (dd, J=11.29, 1.53 Hz, 1H) 7.58 (t, J=7.94 Hz, 1H) 7.22 (d, J=1.83 Hz, 1H) 4.61 (d, J=6.10 Hz, 2H) 3.91 (s, 3H) 1.44 (s, 9H).

### Example 118: 3-(tert-butyl)-N-(2-fluoro-4-(6-(4-methylpiperazin-1-yl)pyrazolo[1,5-a]pyrazin-4-yl)benzyl)-1,2,4-oxadiazole-5-carboxamide

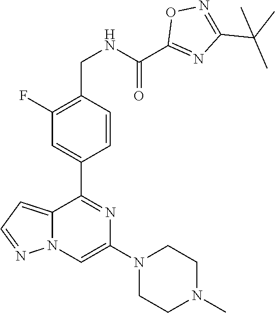 [SMILES: CN1CCN(c2cn3nccc3c(-c3ccc(CNC(=O)c4nc(C(C)(C)C)no4)c(F)c3)n2)CC1]

### 1. Preparation of tert-butyl (4-(6-chloropyrazolo[1,5-a]pyrazin-4-yl)-2-fluorobenzyl)carbamate

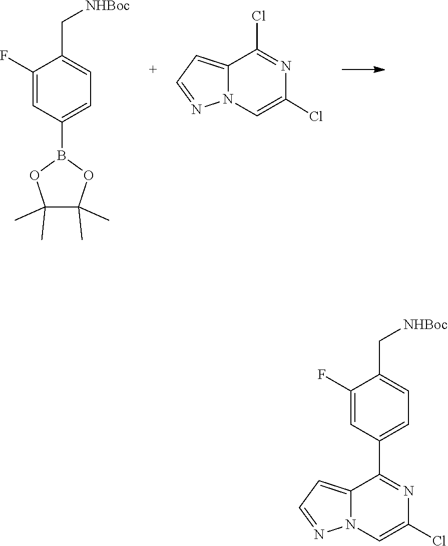 [SMILES: CC(C)(C)OC(=O)NCc1ccc(B2OC(C)(C)C(C)(C)O2)cc1F ; Clc1cn2nccc2c(Cl)n1 ; CC(C)(C)OC(=O)NCc1ccc(-c2nc(Cl)cn3nccc23)cc1F]

A mixture of tert-butyl (2-fluoro-4-(4,4,5,5-tetramethyl-1,3,2-dioxaborolan-2-yl)benzyl)carbamate (500 mg, 1.42 mmol), 4,6-dichloropyrazolo[1,5-a]pyrazine (267.65 mg, 1.42 mmol) and K₃PO₄ (2.0 M, 1.14 mL) were dissolved in dioxane (4.73 mL) and N2 was bubbled through the mixture for 5 min. Pd(t-Bu₃P)₂ (36.38 mg, 71.18 μmol) was added and the reaction mixture was stirred for 2 h at rt. The reaction was concentrated and was purified by column chromatography (gradient elution 0-100% EtOAc:heptane) to afford tert-butyl (4-(6-chloropyrazolo[1,5-a]pyrazin-4-yl)-2-fluorobenzyl)carbamate (460 mg, 86% yield) as a white oily solid. LCMS m/z=377.1 [M+H]⁺

### 2. Preparation of tert-butyl (2-fluoro-4-(6-(4-methylpiperazin-1-yl)pyrazolo[1,5-a]pyrazin-4-yl)benzyl)carbamate

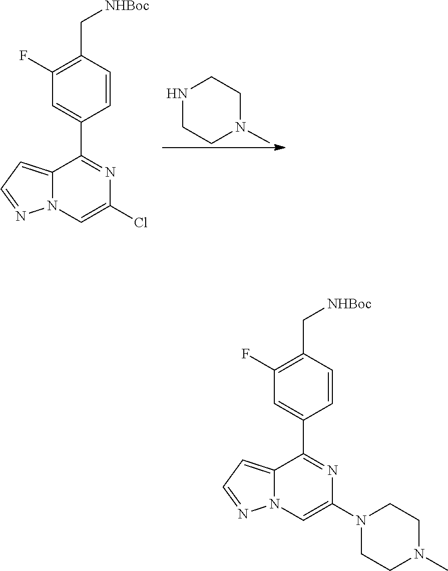

tert-Butyl (2-fluoro-4-(6-(4-methylpiperazin-1-yl)pyrazolo[1,5-a]pyrazin-4-yl)benzyl)carbamate was obtained as a yellow solid, 139 mg, 79.3% yield from tert-butyl (4-(6-chloropyrazolo[1,5-a]pyrazin-4-yl)-2-fluorobenzyl)carbamate and 1-methylpiperazine, following a similar procedure to that described in Example 23, step 2. The crude product was purified by column chromatography (gradient elution 0-100% [3:1 EtOAc:EtOH]:heptane w/1% TEA modifier). LCMS m/z=441.2 [M+H]⁺

### 3. Preparation of (2-fluoro-4-(6-(4-methylpiperazin-1-yl)pyrazolo[1,5-a]pyrazin-4-yl)phenyl)methanamine dihydrochloride

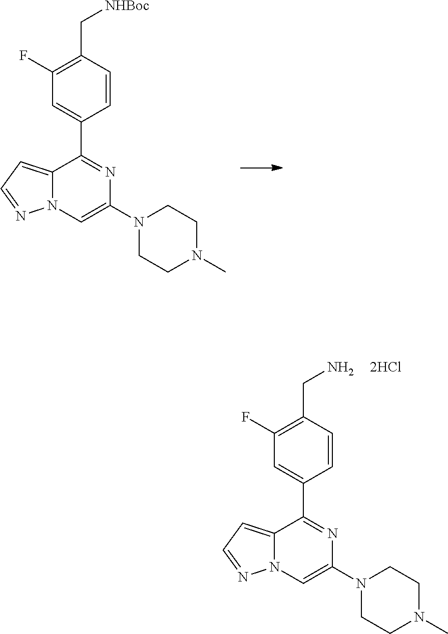 [SMILES: CN1CCN(c2cn3nccc3c(-c3ccc(CNC(=O)OC(C)(C)C)c(F)c3)n2)CC1 ; CN1CCN(c2cn3nccc3c(-c3ccc(CN)c(F)c3)n2)CC1 ; "2HCl"]

(2-Fluoro-4-(6-(4-methylpiperazin-1-yl)pyrazolo[1,5-a]pyrazin-4-yl)phenyl)methanamine dihydrochloride was obtained as a yellow solid, 122 mg, crude, from tert-butyl (2-fluoro-4-(6-(4-methylpiperazin-1-yl)pyrazolo[1,5-a]pyrazin-4-yl)benzyl)carbamate, following the procedure described in Example 23, step 3. LCMS m/z=341.1 [M+H]⁺

### 4. Preparation of 3-(tert-butyl)-N-(2-fluoro-4-(6-(4-methylpiperazin-1-yl)pyrazolo[1,5-a]pyrazin-4-yl)benzyl)-1,2,4-oxadiazole-5-carboxamide

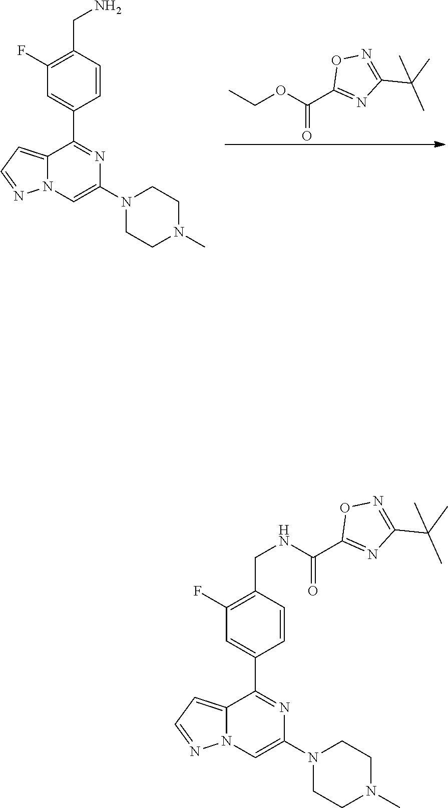 [SMILES: CN1CCN(c2cn3nccc3c(-c3ccc(CN)c(F)c3)n2)CC1 ; CCOC(=O)c1nc(C(C)(C)C)no1 ; CN1CCN(c2cn3nccc3c(-c3ccc(CNC(=O)c4nc(C(C)(C)C)no4)c(F)c3)n2)CC1]

DABAL-Me₃ (33.9 mg, 132.2 μmol) was added to a mixture of (2-fluoro-4-(6-(4-methylpiperazin-1-yl)pyrazolo[1,5-a]pyrazin-4-yl)phenyl)methanamine dihydrochloride (30 mg, 88.1 μmol) and ethyl 3-(tert-butyl)-1,2,4-oxadiazole-5-carboxylate (132.2 μmol) in THF (0.88 mL) and the reaction was heated at 45° C. overnight. The mixture was diluted with NaHCO₃ solution and DCM, and the layers separated. The aqueous layer was extracted with DCM, and the combined organic filtrates were washed with brine, dried over MgSO₄, filtered and concentrated in vacuo. The material was purified by HPLC Method (C3, 5-55%) to afford 3-(tert-butyl)-N-(2-fluoro-4-(6-(4-methylpiperazin-1-yl)pyrazolo[1,5-a]pyrazin-4-yl)benzyl)-1,2,4-oxadiazole-5-carboxamide trifluoroacetate (8.30 mg, 15.5% yield,) as a yellow solid. LCMS m/z=493.2 [M+H]⁺. ¹H NMR (500 MHz, DMSO-d₆) δ ppm 9.97 (br t, J=6.1 Hz, 1H), 9.65 (s, 1H), 8.42 (s, 1H), 8.09 (d, J=2.4 Hz, 1H), 7.95 (br dd, J=7.9, 1.2 Hz, 1H), 7.87 (br dd, J=11.0, 1.8 Hz, 1H), 7.61 (br t, J=7.9 Hz, 1H), 7.14 (br d, J=2.4 Hz, 1H), 4.60 (br d, J=6.1 Hz, 2H), 4.31 (br d, J=12.8 Hz, 2H), 3.54 (br d, J=11.0 Hz, 2H), 3.24-3.06 (m, 2H), 2.86 (br s, 3H), 1.37 (s, 9H).

### Example 119: 5-(tert-butyl)-N-(2-fluoro-4-(6-morpholinopyrazolo[1,5-a]pyrazin-4-yl)benzyl)-1,3,4-oxadiazole-2-carboxamide

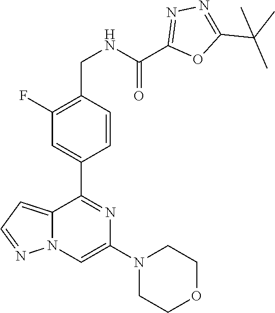 [SMILES: CC(C)(C)c1nnc(C(=O)NCc2ccc(-c3nc(N4CCOCC4)cn4nccc34)cc2F)o1]

### 1. Preparation of tert-butyl (2-fluoro-4-(6-morpholinopyrazolo[1,5-a]pyrazin-4-yl)benzyl)carbamate

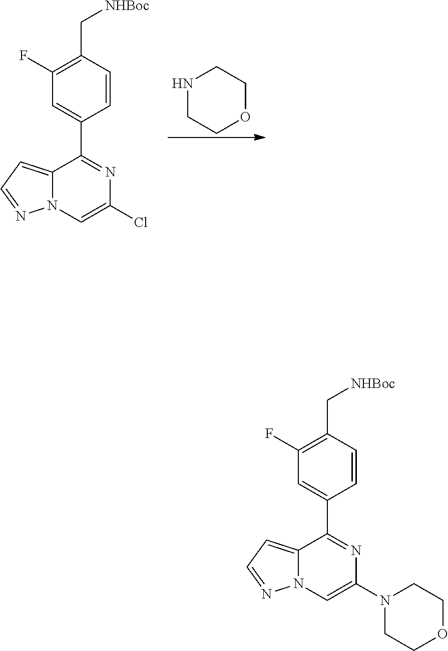 [SMILES: CC(C)(C)OC(=O)NCc1ccc(-c2nc(Cl)cn3nccc23)cc1F ; C1COCCN1 ; CC(C)(C)OC(=O)NCc1ccc(-c2nc(N3CCOCC3)cn3nccc23)cc1F]

tert-Butyl (2-fluoro-4-(6-morpholinopyrazolo[1,5-a]pyrazin-4-yl)benzyl)carbamate was obtained as an orange solid, 163 mg, 75.6% yield from tert-butyl (4-(6-chloropyrazolo[1,5-a]pyrazin-4-yl)-2-fluorobenzyl)carbamate (Example 118, step 1) and morpholine following the procedure described in Example 23, step 2. LCMS m/z=428.2 [M+H]⁺

### 2. Preparation of(2-fluoro-4-(6-morpholinopyrazolo[1,5-a]pyrazin-4-yl)phenyl)methanamine dihydrochloride

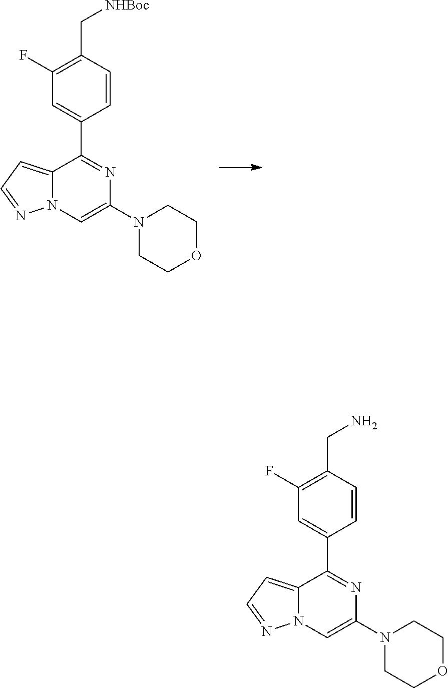 [SMILES: CC(C)(C)OC(=O)NCc1ccc(-c2nc(N3CCOCC3)cn3nccc23)cc1F ; NCc1ccc(-c2nc(N3CCOCC3)cn3nccc23)cc1F]

(2-Fluoro-4-(6-morpholinopyrazolo[1,5-a]pyrazin-4-yl)phenyl)methanamine dihydrochloride was obtained as a red solid, 177 mg, from tert-butyl (2-fluoro-4-(6-morpholinopyrazolo[1,5-a]pyrazin-4-yl)benzyl)carbamate following the procedure described in Example 23, step 3. LCMS m/z=328.1 [M+H]⁺

### 3. Preparation of 5-(tert-butyl)-N-(2-fluoro-4-(6-morpholinopyrazolo[1,5-a]pyrazin-4-yl)benzyl)-1,3,4-oxadiazole-2-carboxamide

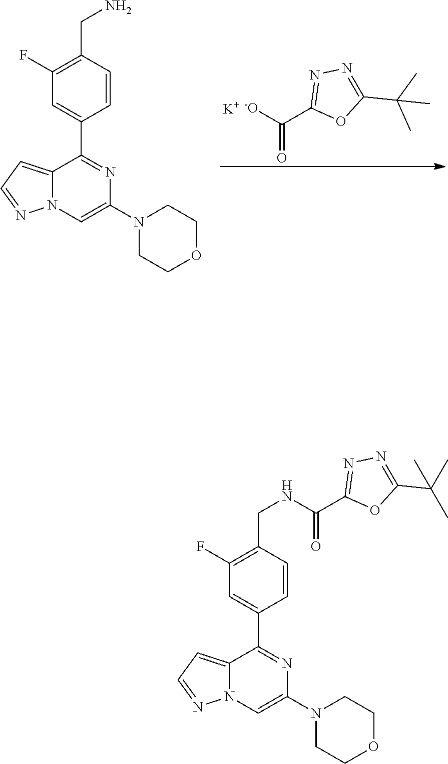

DIPEA (74.0 mg, 572.7 μmol) was added to a mixture of (2-fluoro-4-(6-morpholinopyrazolo[1,5-a]pyrazin-4-yl)phenyl)methanamine dihydrochloride (30 mg, 67.5 μmol), potassium 5-tert-butyl-1,3,4-oxadiazole-2-carboxylate (16.94 mg, 80.9 μmol) and DCM (0.7 mL) and the solution cooled to 0° C. HATU (30.86 mg, 80.9 μmol) was added and the reaction was stirred overnight at rt. The reaction was diluted with water and passed through a phase separator. The aqueous layer was extracted with DCM, and the combined organic layers were concentrated. The crude product was purified by HPLC (Method A2, 5-60%) and lyophilised, to afford 5-(tert-butyl)-N-(2-fluoro-4-(6-morpholinopyrazolo[1,5-a]pyrazin-4-yl)benzyl)-1,3,4-oxadiazole-2-carboxamide (0.3 mg, 0.8% yield) as a yellow solid. LCMS m/z=479.2 [M+H]⁺.

### Example 120: 1-(tert-butyl)-N-(2-methyl-4-(6-morpholinopyrazolo[1,5-a]pyrazin-4-yl)benzyl)-1H-1,2,3-triazole-4-carboxamide

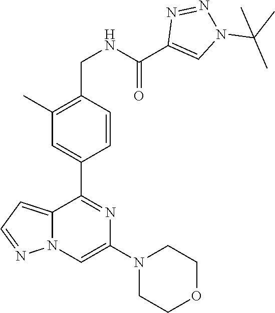 [SMILES: Cc1cc(-c2nc(N3CCOCC3)cn3nccc23)ccc1CNC(=O)c1cn(C(C)(C)C)nn1]

### 1. Preparation of tert-butyl (4-(6-chloropyrazolo[1,5-a]pyrazin-4-yl)-2-methylbenzyl)carbamate

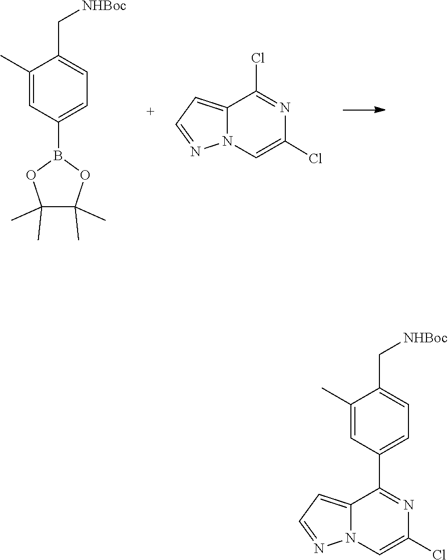

tert-Butyl (4-(6-chloropyrazolo[1,5-a]pyrazin-4-yl)-2-methylbenzyl)carbamate was obtained as a light yellow solid, 913 mg, 85% yield from tert-butyl (2-methyl-4-(4,4,5,5-tetramethyl-1,3,2-dioxaborolan-2-yl)benzyl)carbamate and 4,6-dichloropyrazolo[1,5-a]pyrazine, following the procedure described in Example 118, step 1. LCMS m/z=373.1 [M+H]⁺

### 2. Preparation of tert-butyl (2-methyl-4-(6-morpholinopyrazolo[1,5-a]pyrazin-4-yl)benzyl)carbamate

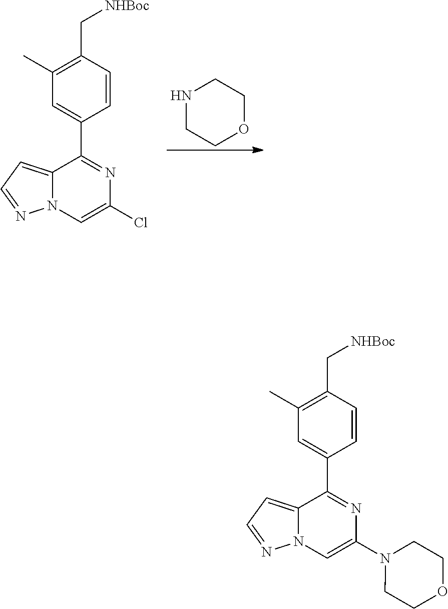 [SMILES: Cc1cc(-c2nc(Cl)cn3nccc23)ccc1CNC(=O)OC(C)(C)C ; C1COCCN1 ; Cc1cc(-c2nc(N3CCOCC3)cn3nccc23)ccc1CNC(=O)OC(C)(C)C]

tert-Butyl (2-methyl-4-(6-morpholinopyrazolo[1,5-a]pyrazin-4-yl)benzyl)carbamate was obtained as a yellow solid, from tert-butyl (4-(6-chloropyrazolo[1,5-a]pyrazin-4-yl)-2-methylbenzyl)carbamate and morpholine, following the procedure described in Example 119, step 1. LCMS m/z=424.3 [M+H]⁺

### 3. Preparation of (2-methyl-4-(6-morpholinopyrazolo[1,5-a]pyrazin-4-yl)phenyl)methanamine dihydrochloride

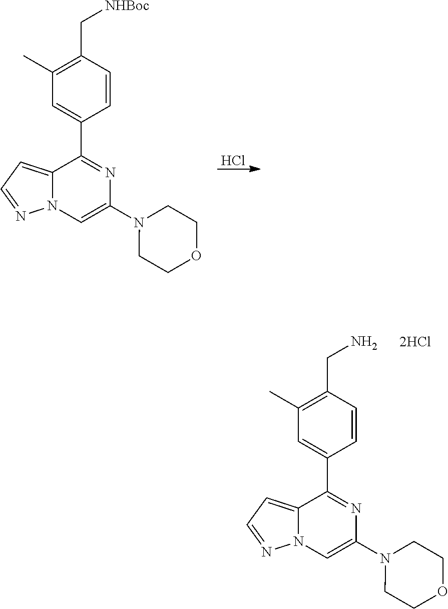 [SMILES: Cc1cc(-c2nc(N3CCOCC3)cn3nccc23)ccc1CNC(=O)OC(C)(C)C ; Cl ; Cc1cc(-c2nc(N3CCOCC3)cn3nccc23)ccc1CN ; "2HCl"]

(2-Methyl-4-(6-morpholinopyrazolo[1,5-a]pyrazin-4-yl)phenyl)methanamine dihydrochloride was obtained as a yellow solid from tert-butyl (2-methyl-4-(6-morpholinopyrazolo[1,5-a]pyrazin-4-yl)benzyl)carbamate following the procedure described in Example 119, step 2. LCMS m/z=324.2 [M+H]⁺

### 4. Preparation of 1-(tert-butyl)-N-(2-methyl-4-(6-morpholinopyrazolo[1,5-a]pyrazin-4-yl)benzyl)-1H-1,2,3-triazole-4-carboxamide trifluoroacetate

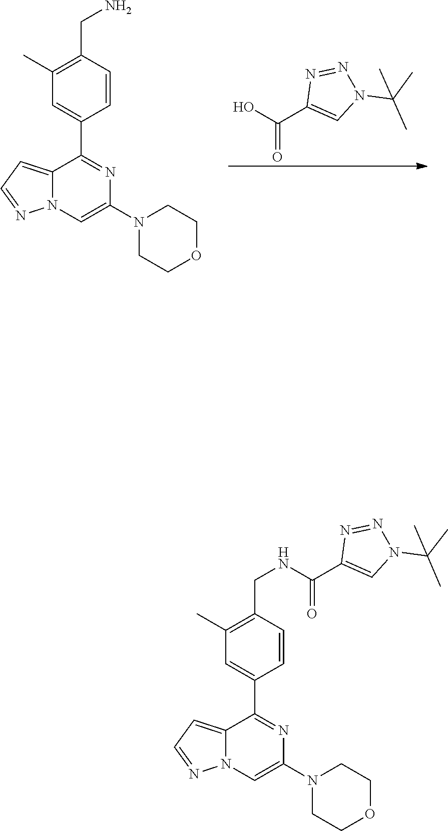 [SMILES: Cc1cc(-c2nc(N3CCOCC3)cn3nccc23)ccc1CN ; CC(C)(C)n1cc(C(=O)O)nn1 ; Cc1cc(-c2nc(N3CCOCC3)cn3nccc23)ccc1CNC(=O)c1cn(C(C)(C)C)nn1]

A vial was charged with (2-methyl-4-(6-morpholinopyrazolo[1,5-a]pyrazin-4-yl)phenyl)methanamine dihydrochloride (40 mg, 100.9 μmol), 1-tert-butyltriazole-4-carboxylic acid (20.49 mg, 121.1 μmol), and DCM (1.01 mL), followed by DIPEA (110.75 mg, 856.9 μmol) and the solution was cooled to 0° C. HATU (46.17 mg, 121.1 μmol) was added and the reaction was stirred overnight at rt. The reaction was loaded directly onto a silica cartridge and purified by column chromatography (gradient elution 0-100% EtOAc:Heptane) to afford the product as a dark orange film. The material was re-purified by HPLC (Method C3, 10-95%) to give 1-(tert-butyl)-N-(2-methyl-4-(6-morpholinopyrazolo[1,5-a]pyrazin-4-yl)benzyl)-1H-1,2,3-triazole-4-carboxamide trifluoroacetate (16 mg, 26.4% yield) as a yellow solid. LCMS m/z=475.2 [M+H]⁺ 1H NMR (500 MHz, DMSO-d₆) δ ppm 9.04 (t, J=6.10 Hz, 1H) 8.71 (s, 1H) 8.13 (s, 1H) 8.01 (d, J=3.05 Hz, 1H) 7.89-7.83 (m, 2H) 7.41 (d, J=8.55 Hz, 1H) 7.03 (d, J=2.44 Hz, 1H) 4.52 (d, J=6.10 Hz, 2H) 3.81-3.74 (m, 4H) 3.38-3.35 (m, 4H) 2.45 (s, 3H) 1.64 (s, 9H)

### Example 121: 1-(tert-butyl)-N-(2-methyl-4-(6-(4-methylpiperazin-1-yl)pyrazolo[1,5-a]pyrazin-4-yl)benzyl)-1H-1,2,3-triazole-4-carboxamide

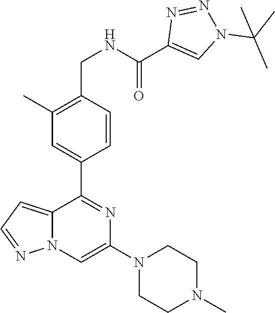 [SMILES: Cc1cc(-c2nc(N3CCN(C)CC3)cn3nccc23)ccc1CNC(=O)c1cn(C(C)(C)C)nn1]

### 1. Preparation of tert-butyl (2-methyl-4-(6-(4-methylpiperazin-1-yl)pyrazolo[1,5-a]pyrazin-4-yl)benzyl)carbamate

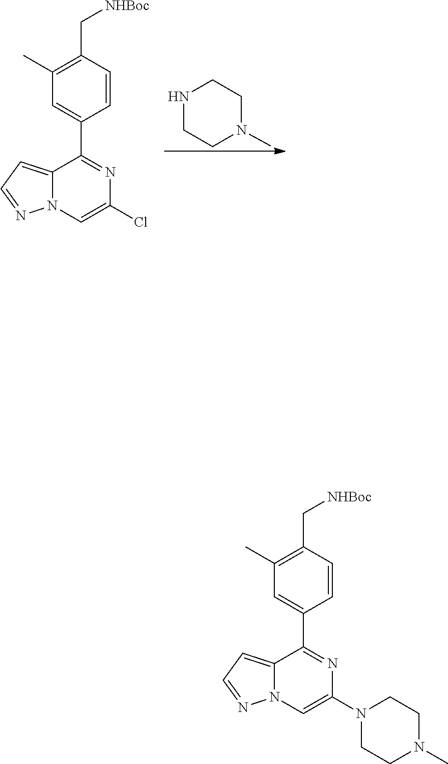

tert-Butyl (2-methyl-4-(6-(4-methylpiperazin-1-yl)pyrazolo[1,5-a]pyrazin-4-yl)benzyl)carbamate was obtained as an orange solid, 232 mg, 66.1% yield, from tert-butyl (4-(6-chloropyrazolo[1,5-a]pyrazin-4-yl)-2-methylbenzyl)carbamate (Example 120, step 1) and 1-methylpiperazine, following the procedure described in Example 119, step 1. The crude product was purified by column chromatography (gradient elution 0-100% [3:1 EtOAc:EtOH]:heptane w/1% TEA modifier). LCMS m/z=437.3 [M+H]⁺

### 2. Preparation of(2-methyl-4-(6-(4-methylpiperazin-1-yl)pyrazolo[1,5-a]pyrazin-4-yl)phenyl)methanamine hydrochloride

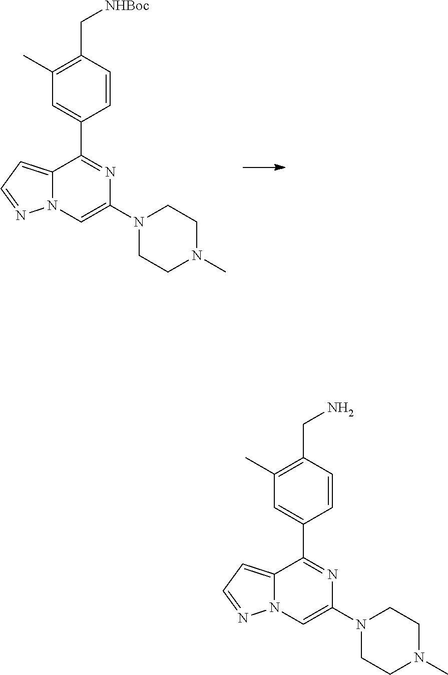 [SMILES: Cc1cc(-c2nc(N3CCN(C)CC3)cn3nccc23)ccc1CNC(=O)OC(C)(C)C ; Cc1cc(-c2nc(N3CCN(C)CC3)cn3nccc23)ccc1CN]

(2-Methyl-4-(6-(4-methylpiperazin-1-yl)pyrazolo[1,5-a]pyrazin-4-yl)phenyl)methanamine hydrochloride was obtained as a yellow solid, 226 mg, crude, from tert-butyl (2-methyl-4-(6-(4-methylpiperazin-1-yl)pyrazolo[1,5-a]pyrazin-4-yl)benzyl)carbamate, following the procedure described in Example 119, step 2. LCMS/z=337.2 [M+H]⁺

### 3. Preparation of 1-(tert-butyl)-N-(2-methyl-4-(6-(4-methylpiperazin-1-yl)pyrazolo[1,5-a]pyrazin-4-yl)benzyl)-1H-1,2,3-triazole-4-carboxamide trifluoroacetate

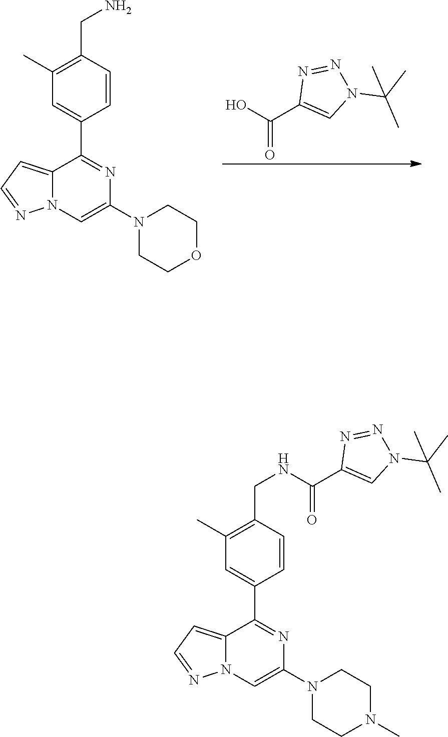 [SMILES: Cc1cc(-c2nc(N3CCOCC3)cn3nccc23)ccc1CN ; CC(C)(C)n1cc(C(=O)O)nn1 ; Cc1cc(-c2nc(N3CCN(C)CC3)cn3nccc23)ccc1CNC(=O)c1cn(C(C)(C)C)nn1]

DIPEA (163.1 mg, 1.26 mmol) was added to a solution of (2-methyl-4-(6-(4-methylpiperazin-1-yl)pyrazolo[1,5-a]pyrazin-4-yl)phenyl)methanamine hydrochloride (50 mg, 148.6 μmol) and 1-tert-butyltriazole-4-carboxylic acid (30.17 mg, 178.3 μmol) in DCM (1.49 mL) and the solution was cooled to 0° C. HATU (68 mg, 178.3 μmol) was added in a single portion and the reaction was stirred overnight at rt. The reaction was diluted with water and passed through a phase separator. The aqueous layer was extracted with DCM, and the combined organic layers were concentrated in vacuo and the residue purified by HPLC (Method C3, 5-50%) afford 1-(tert-butyl)-N-(2-methyl-4-(6-(4-methylpiperazin-1-yl)pyrazolo[1,5-a]pyrazin-4-yl)benzyl)-1H-1,2,3-triazole-4-carboxamide trifluoroacetate (52 mg, 54.7% yield). LCMS m/z=488.3 [M+H]⁺. 1H NMR (500 MHz, DMSO-d₆) δ ppm 9.06 (br t, J=6.1 Hz, 1H), 8.74-8.68 (m, 1H), 8.35 (s, 1H), 8.05 (br d, J=2.4 Hz, 1H), 7.91-7.81 (m, 2H), 7.42 (br d, J=7.9 Hz, 1H), 7.12-7.02 (m, 1H), 4.53 (br d, J=6.1 Hz, 2H), 4.30 (br d, J=13.4 Hz, 2H), 3.54 (br d, J=11.6 Hz, 2H), 3.20 (br d, J=11.6 Hz, 1H), 3.15-3.05 (m, 2H), 2.86 (br d, J=3.7 Hz, 3H), 2.46 (s, 3H), 1.68-1.26 (m, 9H).

### Example 122: 1-(tert-butyl)-N-(4-(6-(2-methoxyethoxy)pyrazolo[1,5-a]pyrazin-4-yl)-2-methylbenzyl)-1H-1,2,3-triazole-4-carboxamide

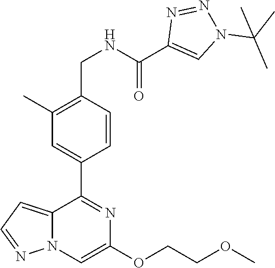 [SMILES: COCCOc1cn2nccc2c(-c2ccc(CNC(=O)c3cn(C(C)(C)C)nn3)c(C)c2)n1]

### 1. Preparation of tert-butyl (4-(6-(2-methoxyethoxy)pyrazolo[1,5-a]pyrazin-4-yl)-2-methylbenzyl)carbamate

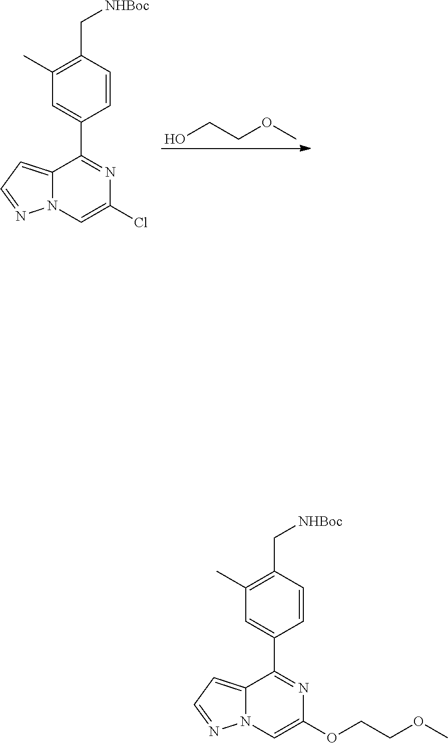

Toluene (7.24 mL) was added to a mixture of tert-butyl (4-(6-chloropyrazolo[1,5-a]pyrazin-4-yl)-2-methylbenzyl)carbamate (Example 120, Step 1, 270 mg, 724.2 μmol), Pd₂(dba)₃ (66.31 mg, 72.4 μmol), Cs₂CO₃ (589.9 mg, 1.81 mmol) and tBuBrettPhos (70.20 mg, 144.8 μmol) and the solution was bubbled with N2 for 5 minutes. 2-Methoxyethanol (165.30 mg, 2.17 mmol) was added and the reaction was stirred overnight at 100° C. The cooled reaction was concentrated and the residue purified by silica gel column chromatography (gradient elution 0-100% EtOAc:Heptane) to afford tert-butyl (4-(6-(2-methoxyethoxy)pyrazolo[1,5-a]pyrazin-4-yl)-2-methylbenzyl)carbamate (155 mg, 51.9% yield) as a yellow solid. LCMS m/z=413.2 [M+H]⁺.

### 2. Preparation of (4-(6-(2-methoxyethoxy)pyrazolo[1,5-a]pyrazin-4-yl)-2-methylphenyl)methanamine dihydrochloride

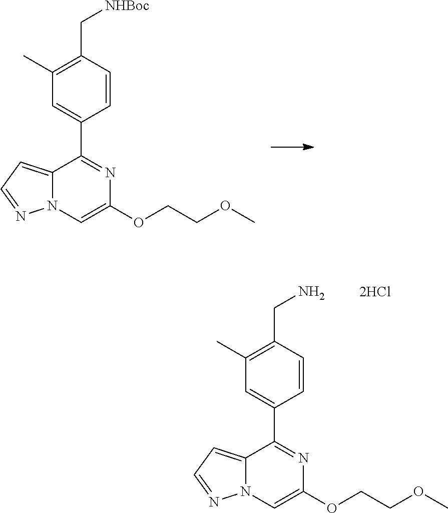 [SMILES: COCCOc1cn2nccc2c(-c2ccc(CNC(=O)OC(C)(C)C)c(C)c2)n1 ; COCCOc1cn2nccc2c(-c2ccc(CN)c(C)c2)n1]

(4-(6-(2-Methoxyethoxy)pyrazolo[1,5-a]pyrazin-4-yl)-2-methylphenyl)methanamine dihydrochloride was obtained as a yellow solid, from tert-butyl (4-(6-(2-methoxyethoxy)pyrazolo[1,5-a]pyrazin-4-yl)-2-methylbenzyl)carbamate, following the method described in Example 23, step 3. LCMS m/z=313.1 [M+H]⁺

### 3. Preparation of 1-(tert-butyl)-N-(4-(6-(2-methoxyethoxy)pyrazolo[1,5-a]pyrazin-4-yl)-2-methylbenzyl)-1H-1,2,3-triazole-4-carboxamide

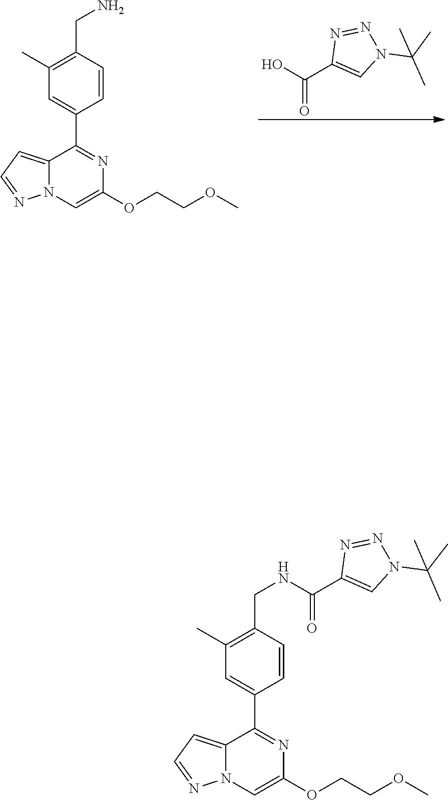 [SMILES: COCCOc1cn2nccc2c(-c2ccc(CN)c(C)c2)n1 ; CC(C)(C)n1cc(C(=O)O)nn1 ; COCCOc1cn2nccc2c(-c2ccc(CNC(=O)c3cn(C(C)(C)C)nn3)c(C)c2)n1]

1-(tert-Butyl)-N-(4-(6-(2-methoxyethoxy)pyrazolo[1,5-a]pyrazin-4-yl)-2-methylbenzyl)-1H-1,2,3-triazole-4-carboxamide was obtained as an off-white solid, 19 mg, 31.2% yield, from (4-(6-(2-methoxyethoxy)pyrazolo[1,5-a]pyrazin-4-yl)-2-methylphenyl)methanamine dihydrochloride and 1-tert-butyltriazole-4-carboxylic acid following a similar procedure to that described in Example 120, step 4. LCMS m/z=464.2 [M+H]^{+ 1}H NMR (500 MHz, DMSO-d₆) δ ppm 9.06 (t, J=6.10 Hz, 1H) 8.72 (s, 1H) 8.42 (d, J=1.22 Hz, 1H) 8.10 (d, J=2.44 Hz, 1H) 7.92-7.84 (m, 2H) 7.43 (d, J=8.55 Hz, 1H), 7.15 (d, J=1.83 Hz, 1H) 4.53 (d, J=6.10 Hz, 2H) 4.45-4.37 (m, 2H) 3.74-3.67 (m, 2H) 3.32 (s, 3H) 2.46 (s, 3H) 1.64 (s, 9H).

### Example 123: 2-(tert-butyl)-N-(2-methyl-4-(6-morpholinopyrazolo[1,5-a]pyrazin-4-yl)benzyl)-2H-1,2,3-triazole-4-carboxamide

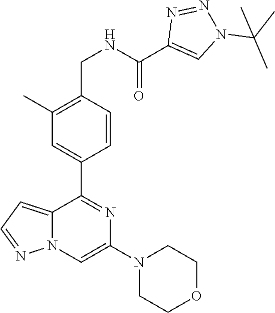 [SMILES: Cc1cc(-c2nc(N3CCOCC3)cn3nccc23)ccc1CNC(=O)c1cn(C(C)(C)C)nn1]

DABAL-Me₃ (47.56 mg, 185.5 μmol) was added to a mixture of (2-methyl-4-(6-morpholinopyrazolo[1,5-a]pyrazin-4-yl)phenyl)methanamine dihydrochloride (Example 120, step 3, 49.02 mg, 123.7 μmol) and ethyl 2-(tert-butyl)-2H-1,2,3-triazole-4-carboxylate (Example 37a, U.S. Ser. No. 10/377,719-B2, 36.6 mg, 185.5 μmol) in THF (1.24 mL) and the reaction was heated at 45° C. overnight. The reaction was carefully quenched with saturated NaHCO₃ solution and diluted with DCM. The mixture was passed through a phase separator, and the aqueous layer was extracted with DCM. The combined organic layers were concentrated and purified by column chromatography (12 g column, gradient elution 0-100% EtOAc:Heptane) to afford a dark orange film. The material was re-purified via reverse phase HPLC (Method A2, 10-95%) and then lyophilised to give 2-(tert-butyl)-N-(2-methyl-4-(6-morpholinopyrazolo[1,5-a]pyrazin-4-yl)benzyl)-2H-1,2,3-triazole-4-carboxamide (1.30 mg, 2.2% yield) as a yellow solid. LCMS m/z=475.2 [M+H]^{+ 1}H NMR (500 MHz, DMSO-d₆) δ ppm 8.91 (t, J=5.80 Hz, 1H) 8.17 (s, 1H) 8.13 (s, 1H) 8.01 (d, J=2.44 Hz, 1H) 7.92-7.83 (m, 2H) 7.42 (d, J=7.94 Hz, 1H) 7.04 (dd, J=2.44, 1.22 Hz, 1H) 4.53 (d, J=6.10 Hz, 2H) 3.82-3.74 (m, 4H) 3.38-3.35 (m, 4H) 2.45 (s, 3H) 1.65 (s, 9H).

### Example 124: 2-(tert-butyl)-N-(2-methyl-4-(6-(4-methylpiperazin-1-yl)pyrazolo[1,5-a]pyrazin-4-yl)benzyl)-2H-1,2,3-triazole-4-carboxamide trifluoroacetate

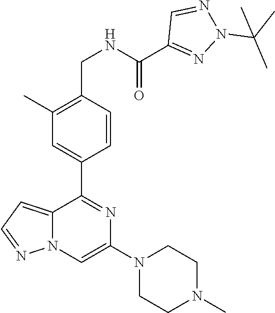 [SMILES: Cc1cc(-c2nc(N3CCN(C)CC3)cn3nccc23)ccc1CNC(=O)c1cnn(C(C)(C)C)n1]

2-(tert-Butyl)-N-(2-methyl-4-(6-(4-methylpiperazin-1-yl)pyrazolo[1,5-a]pyrazin-4-yl)benzyl)-2H-1,2,3-triazole-4-carboxamide trifluoroacetate was obtained, 49 mg, 60.9% yield, from (2-methyl-4-(6-(4-methylpiperazin-1-yl)pyrazolo[1,5-a]pyrazin-4-yl)phenyl)methanamine dihydrochloride (Example 121, step 2) and ethyl 2-(tert-butyl)-2H-1,2,3-triazole-4-carboxylate (Example 37a, U.S. Ser. No. 10/377,719-B2) following the procedure described in Example 118, Step 4. LCMS m/z=488.3 [M+H]^{+ 1}H NMR (500 MHz, DMSO-d₆) δ ppm 8.93 (br t, J=6.1 Hz, 1H), 8.35 (s, 1H), 8.17 (s, 1H), 8.06 (br d, J=2.4 Hz, 1H), 7.91-7.82 (m, 2H), 7.43 (br d, J=7.9 Hz, 1H), 7.08 (d, J=2.4 Hz, 1H), 4.53 (br d, J=6.1 Hz, 2H), 4.30 (br d, J=13.4 Hz, 2H), 3.54 (br d, J=11.6 Hz, 2H), 3.36 (br d, J=3.7 Hz, 2H), 3.25-3.05 (m, 3H), 2.87 (br d, J=3.7 Hz, 2H), 2.46 (s, 3H), 1.68-1.35 (m, 9H)

### Examples 125 to 133

The compounds in the following table were prepared from the appropriate carboxylic acid and amine (as listed below), following a similar procedure to that described in Example 119, step 3.
Amine 4: (2-fluoro-4-(6-morpholinopyrazolo[1,5-a]pyrazin-4-yl)phenyl)methanamine dihydrochloride (Example 119, step 2)
Amine 5: (2-methyl-4-(6-morpholinopyrazolo[1,5-a]pyrazin-4-yl)phenyl)methanamine dihydrochloride (Example 120, step 3)
Amine 6: (2-fluoro-4-(6-(4-methylpiperazin-1-yl)pyrazolo[1,5-a]pyrazin-4-yl)phenyl)methanamine dihydrochloride (Example 118, step 3)
Amine 7: (2-methyl-4-(6-(4-methylpiperazin-1-yl)pyrazolo[1,5-a]pyrazin-4-yl)phenyl)methanamine dihydrochloride (Example 121, step 2)
Amine 8: (4-(6-(2-methoxyethoxy)pyrazolo[1,5-a]pyrazin-4-yl)-2-methylphenyl)methanamine dihydrochloride (Example 122, step 2)

| Example No | Name, Structure, Starting Materials (SM), Data |
|---|---|
| 125 | 2-(tert-butyl)-N-(2-fluoro-4-(6-morpholinopyrazolo[1,5-a]pyrazin-4- yl)benzyl)oxazole-4-carboxamide 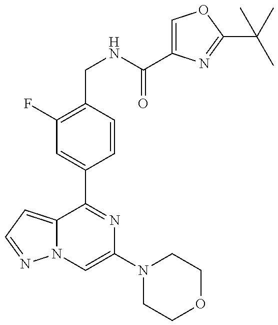 [SMILES: CC(C)(C)c1nc(C(=O)NCc2ccc(-c3nc(N4CCOCC4)cn4nccc34)cc2F)co1] 2-(tert-butyl)oxazole-4-carboxylic acid and Amine 4. 2 mg, 5.6% yield LCMS m/z = 479.2 [M + H]⁺. 1H NMR (500 MHz, DMSO-d₆) δ ppm 8.73 (br t, J = 6.1 Hz, 1 H), 8.55 (s, 1 H), 8.19 (s, 1 H), 8.04 (d, J = 2.4 Hz, 1 H), 8.02- 7.95 (m, 1 H), 7.95-7.77 (m, 4 H), 7.56-7.45 (m, 3 H), 7.09 (br d, J = 1.8 Hz, 1 H), 4.56 (br d, J = 6.1 Hz, 2 H), 3.82-3.72 (m, 4 H), 1.37 (s, 9 H). |
| 126 | N-(2-methyl-4-(6-morpholinopyrazolo[1,5-a]pyrazin-4-yl)benzyl)-5-(1- methylcyclopropyl)-1,2,4-oxadiazole-3-carboxamide 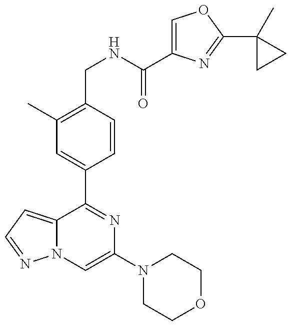 [SMILES: Cc1cc(-c2nc(N3CCOCC3)cn3nccc23)ccc1CNC(=O)c1coc(C2(C)CC2)n1] potassium 5-(1-methylcyclopropyl)-1,2,4-oxadiazole-3-carboxylate and Amine 5. yellow film, 10 mg, 19.9% yield. LCMS m/z = 474.2 [M + H]⁺ ¹H NMR (500 MHz, DMSO-d₆) δ ppm 9.44 (t, J = 6.10 Hz, 1 H) 8.14 (s, 1 H) 8.02 (d, J = 2.44 Hz, 1 H) 7.90-7.85 (m, 2 H) 7.41 (d, J = 7.33 Hz, 1 H) 7.04 (d, J = 2.44 Hz, 1 H) 4.52 (d, J = 6.10 Hz, 2 H) 3.81-3.75 (m, 4H) 3.39-3.35 (m, 4 H) 2.44 (s, 3 H) 1.54 (s, 3 H) 1.41-1.36 (m, 2 H) 1.20-1.15 (m, 2 H). |
| 127 | 5-(tert-butyl)-N-(2-fluoro-4-(6-(4-methylpiperazin-1-yl)pyrazolo[1,5- alpyrazin-4-yl)benzyl)-1,3,4-oxadiazole-2-carboxamide 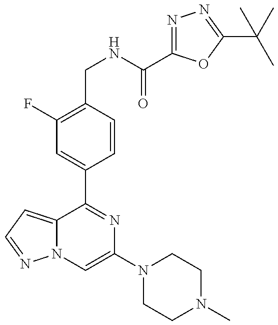 [SMILES: CN1CCN(c2cn3nccc3c(-c3ccc(CNC(=O)c4nnc(C(C)(C)C)o4)c(F)c3)n2)CC1] 5-tert-butyl-1,3,4-oxadiazole-2-carboxylic acid and Amine 6 yellow solid, 6.3 mg, 14.5% yield, LCMS m/z = 493.2 [M + H]⁺ ¹H NMR (500 MHz, DMSO-d₆) δ ppm 9.95-9.87 (m, 1 H), 8.39 (s, 1 H), 8.08 (br d, J = 2.4 Hz, 1 H), 7.94 (br dd, J = 7.9, 1.2 Hz, 1 H), 7.86 (br d, J = 11.0 Hz, 1 H), 7.65-7.56 (m, 1 H), 7.12 (s, 1 H), 4.60 (br d, J = 6.1 Hz, 2 H), 3.70- 3.40 (m, 5 H), 2.84 (s, 4 H), 1.40 (s, 9 H) |
| 128^{A} | 1-(tert-butyl)-N-(2-fluoro-4-(6-(4-methylpiperazin-1-yl)pyrazolo[1,5- a]pyrazin-4-yl)benzyl)-1H-1,2,3-triazole-4-carboxamide trifluoroacetate 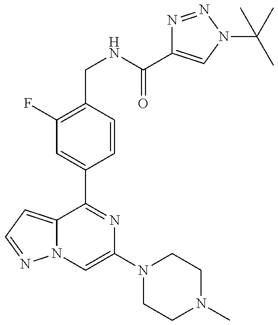 [SMILES: CN1CCN(c2cn3nccc3c(-c3ccc(CNC(=O)c4cn(C(C)(C)C)nn4)c(F)c3)n2)CC1] 1-tert-butyltriazole-4-carboxylic acid and Amine 6 20 mg, 36.4% yield. LCMS m/z = 492.3 [M + H]⁺. ¹H NMR (500 MHz, DMSO-d₆) δ ppm 9.64 (s, 1 H), 9.16 (br t, J = 6.4 Hz, 1 H), 8.72 (s, 1 H), 8.41 (s, 1 H), 8.08 (d, J = 2.4 Hz, 1 H), 7.93 (br dd, J = 7.9, 1.8 Hz, 1 H), 7.86 (br dd, J = 11.0, 1.8 Hz, 1 H), 7.54 (br t, J = 7.9 Hz, 1 H), 7.14 (br d, J = 3.7 Hz, 1 H), 4.60 (br d, J = 6.1 Hz, 2 H), 4.31 (br d, J = 13.4 Hz, 2 H), 3.54 (br d, J = 11.6 Hz, 3 H), 3.35 (br s, 1 H), 3.25-3.09 (m, 3 H), 2.87 (br d, J = 4.3 Hz, 3 H), 1.64 (s, 9 H) |
| 129^{A} | 2-(tert-butyl)-N-(2-fluoro-4-(6-(4-methylpiperazin-1-yl)pyrazolo[1,5- alpyrazin-4-yl)benzyl)oxazole-4-carboxamide trifluoroacetate 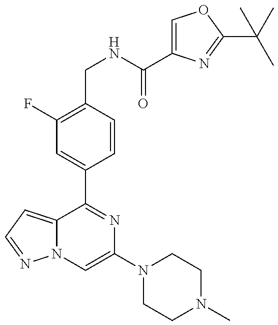 [SMILES: CN1CCN(c2cn3nccc3c(-c3ccc(CNC(=O)c4coc(C(C)(C)C)n4)c(F)c3)n2)CC1] Amine 6 and 2-tert-butyloxazole-4-carboxylic acid yellow solid. LCMS m/z = 492.2 [M + H]⁺. 1H NMR (500 MHz, DMSO-d₆) δ ppm 9.64 (s, 1 H), 8.75 (br t, J = 6.1 Hz, 1 H), 8.55 (s, 1 H), 8.41 (s, 1 H), 8.09 (d, J = 2.4 Hz, 1 H), 7.94 (br dd, J = 7.9, 1.2 Hz, 1 H), 7.85 (br dd, J = 11.0, 1.2 Hz, 1 H), 7.53 (br t, J = 7.9 Hz, 1 H), 7.12-7.17 (m, 1 H), 4.56 (br d, J = 6.1 Hz, 2 H), 4.31 (br d, J = 12.8 Hz, 2 H), 3.54 (br d, J = 11.0 Hz, 4 H), 3.36 (br s, 2 H), 3.26-3.05 (m, 4 H), 2.87 (br d, J = 3.7 Hz, 3 H), 1.44-1.15 (m, 9 H). |
| 130^{A} | 2-(tert-butyl)-N-(2-methyl-4-(6-(4-methylpiperazin-1-yl)pyrazolo[1,5- alpyrazin-4-yl)benzyl)-2H-tetrazole-5-carboxamide 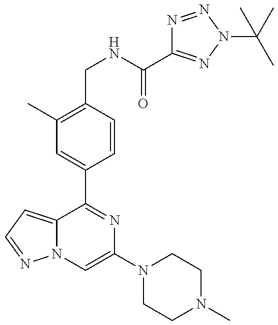 [SMILES: Cc1cc(-c2nc(N3CCN(C)CC3)cn3nccc23)ccc1CNC(=O)c1nnn(C(C)(C)C)n1] Amine 7 and 2-(tert-butyl)-2H-tetrazole-5-carboxylic acid 43 mg, 45.6% yield, LCMS m/z = 489.3 [M + H]⁺. 1H NMR (500 MHz, DMSO-d₆) δ ppm 9.58 (br t, J = 6.1 Hz, 1 H), 8.35 (s, 1 H), 8.06 (br d, J = 3.1 Hz, 1 H), 7.92-7.73 (m, 2 H), 7.44 (br d, J = 8.5 Hz, 1 H), 7.08 (br d, J = 3.7 Hz, 1 H), 4.56 (br d, J = 6.1 Hz, 2 H), 4.30 (br d, J = 12.8 Hz, 2 H), 3.54 (br d, J = 11.6 Hz, 2 H), 3.35 (s, 3 H), 3.17-3.05 (m, 2 H), 2.86 (br d, J = 3.7 Hz, 2 H), 2.47 (s, 3 H), 1.74 (s, 9 H) |
| 131^{A} | N-(2-methyl-4-(6-(4-methylpiperazin-1-yl)pyrazolo[1,5-a]pyrazin-4- yl)benzyl)-5-(1-methylcyclopropyl)-1,2,4-oxadiazole-3-carboxamide trifluoroacetate 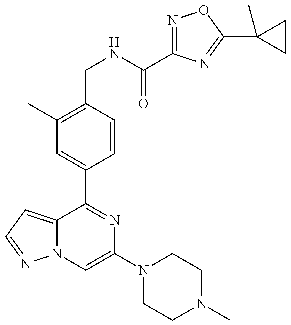 [SMILES: Cc1cc(-c2nc(N3CCN(C)CC3)cn3nccc23)ccc1CNC(=O)c1noc(C2(C)CC2)n1] Amine 7 and potassium 5-(1-methylcyclopropyl)-1,2,4-oxadiazole-3- carboxylate 41 mg, 43.6% yield. LCMS m/z = 487.2 [M + H]⁺. ¹H NMR (500 MHz, DMSO-d₆) δ ppm 9.45 (br t, J = 6.1 Hz, 1 H), 8.36 (s, 1 H), 8.06 (d, J = 2.4 Hz, 1 H), 7.94-7.78 (m, 2 H), 7.42 (br d, J = 7.9 Hz, 1H), 7.08 (s, 1 H), 4.52 (br d, J = 6.1 Hz, 2 H), 4.30 (br d, J = 13.4 Hz, 2 H), 3.54 (br d, J = 11.6 Hz, 2 H), 3.39- 3.28 (m, 1 H), 3.26-3.04 (m, 2 H), 2.87 (br d, J = 3.7 Hz, 1 H), 2.45 (s, 1 H), 1.55 (s, 3 H), 1.41-1.30 (m, 2 H), 1.22-1.08 (m, 2 H) |
| 132 | 5-(tert-butyl)-N-(4-(6-(2-methoxyethoxy)pyrazolo[1,5-a]pyrazin-4-yl)-2- methylbenzyl)-1,2,4-oxadiazole-3-carboxamide 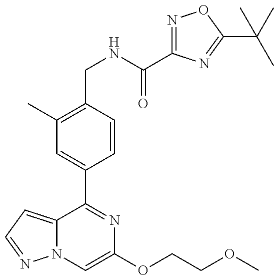 [SMILES: COCCOc1cn2nccc2c(-c2ccc(CNC(=O)c3noc(C(C)(C)C)n3)c(C)c2)n1] Amine 8 and 5-(1-methylcyclopropyl)-1,2,4-oxadiazole-3-carboxylic acid a yellow film, 8 mg, 13.1% yield, LCMS m/z = 463.2 [M + H]⁺ ¹H NMR (500 MHz, DMSO-d₆) δ ppm 9.45 (t, J = 5.80 Hz, 1 H) 8.43 (s, 1 H) 8.11 (d, J = 2.44 Hz, 1 H) 7.92-7.87 (m, 2 H) 7.43 (d, J = 8.55 Hz, 1 H) 7.15 (d, J = 2.44 Hz, 1 H) 4.52 (d, J-6.10 Hz, 2 H) 4.44-4.38 (m, 2H) 3.76-3.68 (m, 2 H) 3.33 (s, 3 H) 2.45 (s, 3 H) 1.55 (s, 3 H) 1.42-1.37 (m, 2 H) 1.19-1.15 (m, 2 H) |
| 133 | N-(4-(6-(2-methoxyethoxy)pyrazolo[1,5-a]pyrazin-4-yl)-2-methylbenzyl)-3- (1-methylcyclopropyl)-1,2,4-oxadiazole-5-carboxamide 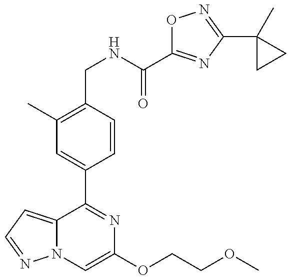 [SMILES: COCCOc1cn2nccc2c(-c2ccc(CNC(=O)c3nc(C4(C)CC4)no3)c(C)c2)n1] Amine 8 and 3-(1-methylcyclopropyl)-1,2,4-oxadiazole-5-carboxylic acid, yellow film, 1 mg, 1.7% yield, LCMS m/z = 463.2 [M + H]⁺ ¹H NMR (500 MHz, DMSO-d₆) δ ppm 9.86-9.78 (m, 1 H) 8.43 (s, 1 H) 8.11 (d, J = 2.44 Hz, 1 H) 7.89 (br s, 2H) 7.48-7.43 (m, 1 H) 7.15 (d, J = 2.44 Hz, 1 H) 4.53 (d, J = 6.10 Hz, 2 H) 4.44-4.38 (m, 2 H) 3.75-3.68 (m, 1 H) 3.44- 3.34 (m, 3 H) 2.45 (s, 3 H) 1.49 (s, 3 H) 1.22-1.17 (m, 2 H) 1.01-0.98 (m, 2 H) |

### A-purified by HPLC (Method C3, 5-45%)

### Example 134: N-(4-(6-(azetidin-1-yl)pyrazolo[1,5-a]pyrazin-4-yl)benzyl)-5-(tert-butyl)-1,2,4-oxadiazole-3-carboxamide

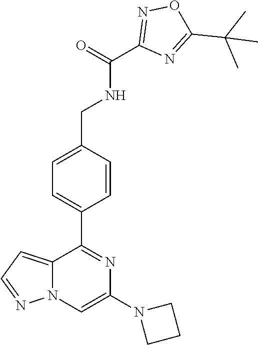 [SMILES: CC(C)(C)c1nc(C(=O)NCc2ccc(-c3nc(N4CCC4)cn4nccc34)cc2)no1]

### 1. Preparation of tert-butyl (4-(6-(azetidin-1-yl)pyrazolo[1,5-a]pyrazin-4-yl)benzyl)carbamate

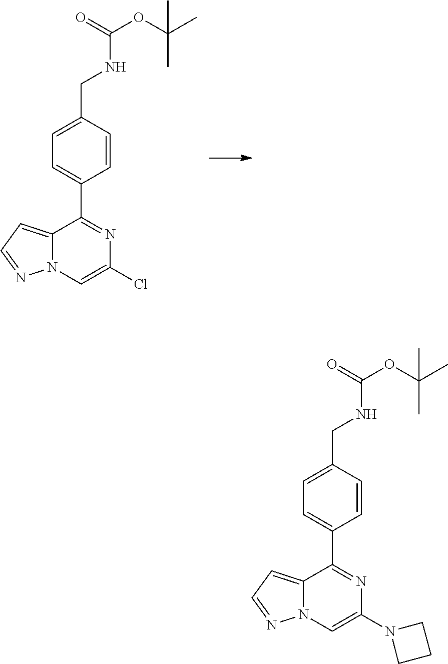 [SMILES: CC(C)(C)OC(=O)NCc1ccc(-c2nc(Cl)cn3nccc23)cc1 ; CC(C)(C)OC(=O)NCc1ccc(-c2nc(N3CCC3)cn3nccc23)cc1]

A mixture of tert-butyl (4-(6-chloropyrazolo[1,5-a]pyrazin-4-yl)benzyl)carbamate (Example 23, Step 1, 200 mg, 557.4 μmol), RuPhos Pd G3 (69.93 mg, 83.6 μmol), azetidine hydrochloride (208.59 mg, 2.23 mmol), and NaOtBu (267.82 mg, 2.79 mmol) in toluene (4 mL) was stirred at 95° C. for 8 h under N2. The cooled mixture was concentrated in vacuum and the crude was purified by column chromatography on silica gel eluted with (PE/EtOAc=I/O to 2/1) to give tert-butyl (4-(6-(azetidin-1-yl)pyrazolo[1,5-a]pyrazin-4-yl)benzyl)carbamate (160 mg, 75.7% yield) as a yellow solid. LCMS m/z=380.1 [M+H]⁺

### 2. Preparation of(4-(6-(azetidin-1-yl)pyrazolo[1,5-a]pyrazin-4-yl)phenyl)methanamine trifluoroacetate

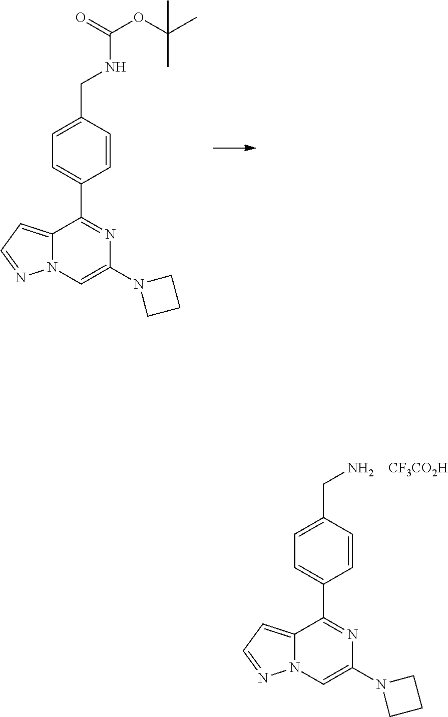 [SMILES: CC(C)(C)OC(=O)NCc1ccc(-c2nc(N3CCC3)cn3nccc23)cc1 ; NCc1ccc(-c2nc(N3CCC3)cn3nccc23)cc1 ; "CF3CO2H"]

To a solution of tert-butyl (4-(6-(azetidin-1-yl)pyrazolo[1,5-a]pyrazin-4-yl)benzyl)carbamate (140 mg, 368.96 μmol) in DCM (12 mL) was added TFA (4 mL) dropwise and the mixture was stirred at 30° C. for 1 h. The mixture was concentrated in vacuum to give (4-(6-(azetidin-1-yl)pyrazolo[1,5-a]pyrazin-4-yl)phenyl)methanamine trifluoroacetate (100 mg, crude) as a yellow solid, which was used in next step without further purification. LCMS m/z=280.0 [M+H]⁺

### 3. Preparation of N-(4-(6-(azetidin-1-yl)pyrazolo[1,5-a]pyrazin-4-yl)benzyl)-5-(tert-butyl)-1,2,4-oxadiazole-3-carboxamide

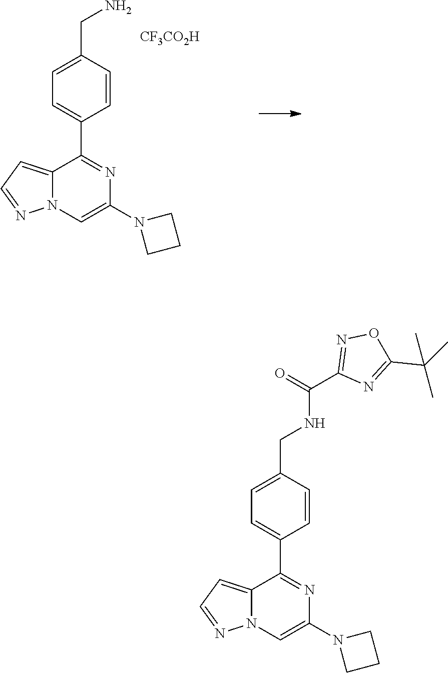 [SMILES: NCc1ccc(-c2nc(N3CCC3)cn3nccc23)cc1 ; "CF3CO2H" ; CC(C)(C)c1nc(C(=O)NCc2ccc(-c3nc(N4CCC4)cn4nccc34)cc2)no1]

To a solution of (4-(6-(azetidin-1-yl)pyrazolo[1,5-a]pyrazin-4-yl)phenyl)methanamine trifluoroacetate (80.0 mg, 286.4 μmol) in DCM (50 mL) was added DIPEA (111.04 mg, 859.2 μmol), then 5-tert-butyl-1,2,4-oxadiazole-3-carbonyl chloride (Example 82, step 3, 108.03 mg, 572.8 μmol) and the mixture was stirred at 20° C. for 1 h. The mixture was concentrated in vacuum and the crude was purified by Prep-HPLC (Method E, 42-72% gradient) to give N-(4-(6-(azetidin-1-yl)pyrazolo[1,5-a]pyrazin-4-yl)benzyl)-5-(tert-butyl)-1,2,4-oxadiazole-3-carboxamide (27.10 mg, 21.9% yield) as a yellow solid. LCMS m/z=432.1 [M+H]⁺ 1H NMR (400 MHz, DMSO-d₆) δ: 9.57-9.56 (m, 1H), 8.01-7.98 (m, 2H), 7.95 (d, J=2.8 Hz, 1H), 7.85 (s, 1H), 7.50 (d, J=8.8 Hz, 2H), 6.99-6.98 (m, 1H), 4.53 (d, J=6.0 Hz, 2H), 3.92 (t, J=7.2 Hz, 4H), 2.36-2.33 (m, 2H), 1.42 (s, 9H).

### Example 135: 5-(tert-butyl)-N-(4-(6-(dimethylamino)pyrazolo[1,5-a]pyrazin-4-yl)benzyl)-1,2,4-oxadiazole-3-carboxamide

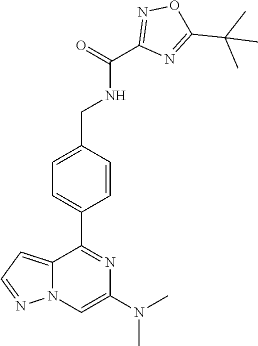 [SMILES: CN(C)c1cn2nccc2c(-c2ccc(CNC(=O)c3noc(C(C)(C)C)n3)cc2)n1]

### 1. Preparation of tert-butyl (4-(6-(dimethylamino)pyrazolo[1,5-a]pyrazin-4-yl)benzyl)carbamate

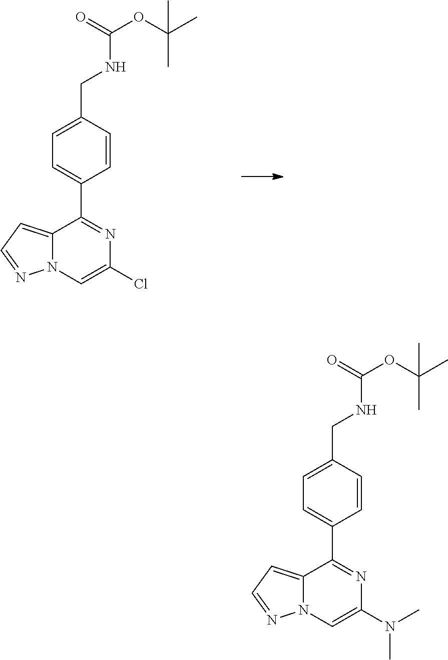 [SMILES: CC(C)(C)OC(=O)NCc1ccc(-c2nc(Cl)cn3nccc23)cc1 ; CN(C)c1cn2nccc2c(-c2ccc(CNC(=O)OC(C)(C)C)cc2)n1]

Tert-butyl (4-(6-(dimethylamino)pyrazolo[1,5-a]pyrazin-4-yl)benzyl)carbamate was obtained (60 mg, 25.0%) from methanamine and tert-butyl (4-(6-chloropyrazolo[1,5-a]pyrazin-4-yl)benzyl)carbamate (Example 23, Step 1) following a similar procedure to that described in

### Example 134, step 1. LCMS m/z=368.1 [M+H]⁺

### 2. Preparation of 4-(4-(aminomethyl)phenyl)-N,N-dimethylpyrazolo[1,5-a]pyrazin-6-amine dihydrochloride

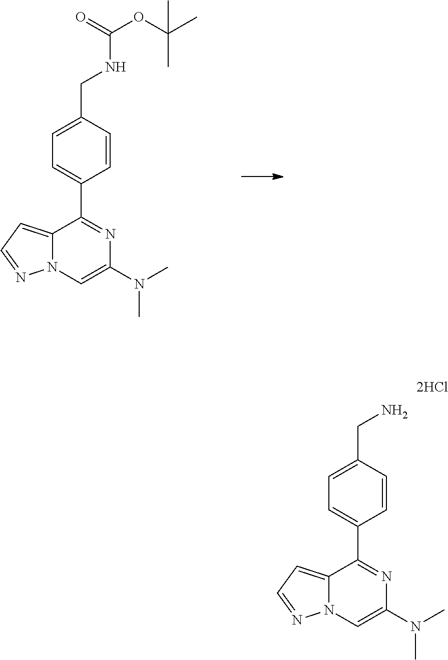 [SMILES: CN(C)c1cn2nccc2c(-c2ccc(CNC(=O)OC(C)(C)C)cc2)n1 ; CN(C)c1cn2nccc2c(-c2ccc(CN)cc2)n1]

HCl/EtOAc (10.0 mL) was added to tert-butyl (4-(6-(dimethylamino)pyrazolo[1,5-a]pyrazin-4-yl)benzyl)carbamate (60.0 mg, 163.3 μmol) and the reaction mixture was stirred at 30° C. for 2 h. The mixture was concentrated in vacuum to give 4-(4-(aminomethyl)phenyl)-N,N-dimethylpyrazolo[1,5-a]pyrazin-6-amine dihydrochloride (40 mg, crude) as a gray solid which was used for the next step without further purification. LCMS m/z=268.0 [M+H]⁺

### 3. Preparation of N-(4-(6-(azetidin-1-yl)pyrazolo[1,5-a]pyrazin-4-yl)benzyl)-5-(tert-butyl)-1,2,4-oxadiazole-3-carboxamide

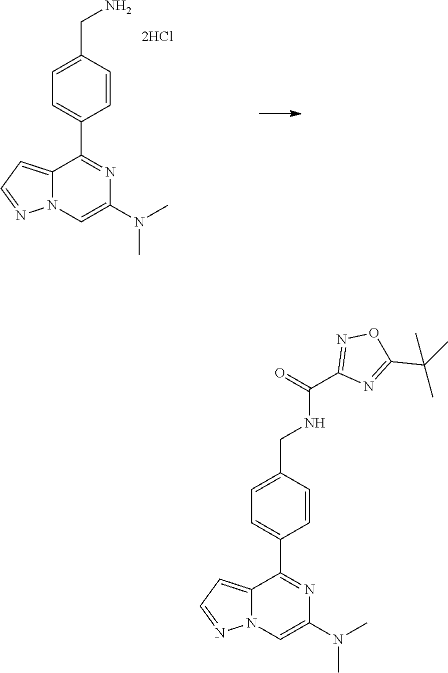 [SMILES: CN(C)c1cn2nccc2c(-c2ccc(CN)cc2)n1 ; "2HCl" ; CN(C)c1cn2nccc2c(-c2ccc(CNC(=O)c3noc(C(C)(C)C)n3)cc2)n1]

5-Tert-butyl-1,2,4-oxadiazole-3-carbonyl chloride (Example 82, step 3, 37.25 mg, 197.5 μmol) was added to a solution of 4-(4-(aminomethyl)phenyl)-N,N-dimethylpyrazolo[1,5-a]pyrazin-6-amine dihydrochloride (40 mg, 131.7 μmol) and DIPEA (68.07 mg, 526.7 μmol) in DCM (45 mL) and the reaction was stirred at 30° C. for 3 h. The mixture was concentrated in vacuum and the residue was poured into water (120 mL). The aqueous was extracted with DCM (70 mL×3), the combined organic layers were dried over Na₂SO₄ and concentrated in vacuum. The crude product was purified by Prep-HPLC (Method D, 55-75% gradient) to give N-(4-(6-(azetidin-1-yl)pyrazolo[1,5-a]pyrazin-4-yl)benzyl)-5-(tert-butyl)-1,2,4-oxadiazole-3-carboxamide (30.0 mg, 47.8% yield) as a pale white solid. LCMS m/z=420.1 [M+H]⁺

¹H NMR (400 MHz, DMSO-d₆) δ: 9.57-9.56 (m, 1H), 8.01-7.98 (m, 2H), 7.95 (d, J=2.8 Hz, 1H), 7.85 (s, 1H), 7.50 (d, J=8.8 Hz, 2H), 6.99-6.98 (m, 1H), 4.53 (d, J=6.0 Hz, 2H), 3.92 (t, J=7.2 Hz, 4H), 2.36-2.33 (m, 2H), 1.42 (s, 9H).

### Example 136: 5-(tert-butyl)-N-((1-(6-(1-methyl-1H-pyrazol-4-yl)pyrazolo[1,5-a]pyrazin-4-yl)piperidin-4-yl)methyl)-1,2,4-oxadiazole-3-carboxamide

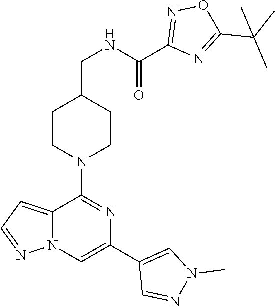 [SMILES: Cn1cc(-c2cn3nccc3c(N3CCC(CNC(=O)c4noc(C(C)(C)C)n4)CC3)n2)cn1]

### 1. Preparation of tert-butyl ((1-(6-(1-methyl-1H-pyrazol-4-yl)pyrazolo[1,5-a]pyrazin-4-yl)piperidin-4-yl)methyl)carbamate

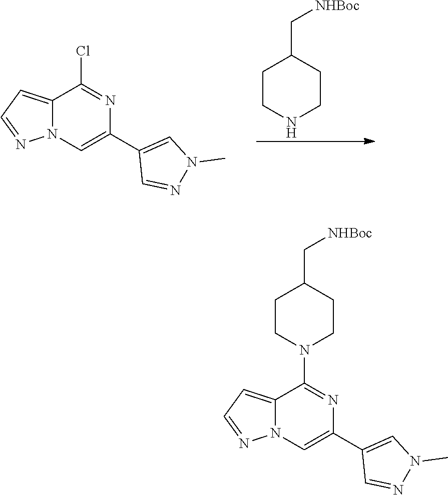 [SMILES: Cn1cc(-c2cn3nccc3c(Cl)n2)cn1 ; CC(C)(C)OC(=O)NCC1CCNCC1 ; Cn1cc(-c2cn3nccc3c(N3CCC(CNC(=O)OC(C)(C)C)CC3)n2)cn1]

A mixture of tert-butyl N-(4-piperidylmethyl)carbamate 0.06 mg, 513.6 μmol), 4-chloro-6-(1-methylpyrazol-4-yl)pyrazolo[1,5-a]pyrazine (60.0 mg, 256.8 μmol), and Cs₂CO₃ (167.33 mg, 513.56 μmol) were dissolved in DMSO (0.856 mL) and the reaction was stirred overnight at rt. The reaction was concentrated and purified by column chromatography (12 g column, gradient elution 0-75% [3:1 EtOAc:EtOH]:Heptane) to afford tert-butyl ((1-(6-(1-methyl-1H-pyrazol-4-yl)pyrazolo[1,5-a]pyrazin-4-yl)piperidin-4-yl)methyl)carbamate (100 mg, 94.6% yield) as a light brown solid. LCMS m/z=412.3 [M+H]⁺

### 2. Preparation of(1-(6-(1-methyl-1H-pyrazol-4-yl)pyrazolo[1,5-a]pyrazin-4-yl)piperidin-4-yl)methanamine dihydrochloride

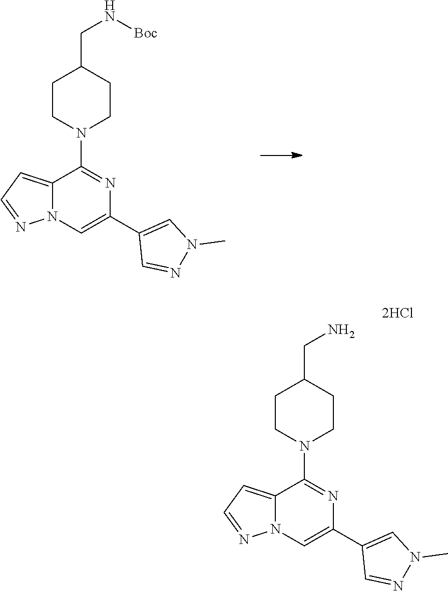 [SMILES: Cn1cc(-c2cn3nccc3c(N3CCC(CNC(=O)OC(C)(C)C)CC3)n2)cn1 ; Cn1cc(-c2cn3nccc3c(N3CCC(CN)CC3)n2)cn1]

tert-Butyl ((1-(6-(1-methyl-1H-pyrazol-4-yl)pyrazolo[1,5-a]pyrazin-4-yl)piperidin-4-yl)methyl)carbamate (82 mg, 199.3 μmol) was dissolved in MeOH (1.99 mL) and HCl solution (1.25 M in MeOH, 1.59 mL) was added and the reaction was stirred overnight at 50° C. The reaction was concentrated and vacuum dried to afford (1-(6-(1-methyl-1H-pyrazol-4-yl)pyrazolo[1,5-a]pyrazin-4-yl)piperidin-4-yl)methanamine dihydrochloride (93 mg, crude) as a red solid. LCMS m/z=312.2 [M+H]⁺

### 3. Preparation of 5-(tert-butyl)-N-((1-(6-(1-methyl-1H-pyrazol-4-yl)pyrazolo[1,5-a]pyrazin-4-yl)piperidin-4-yl)methyl)-1,2,4-oxadiazole-3-carboxamide

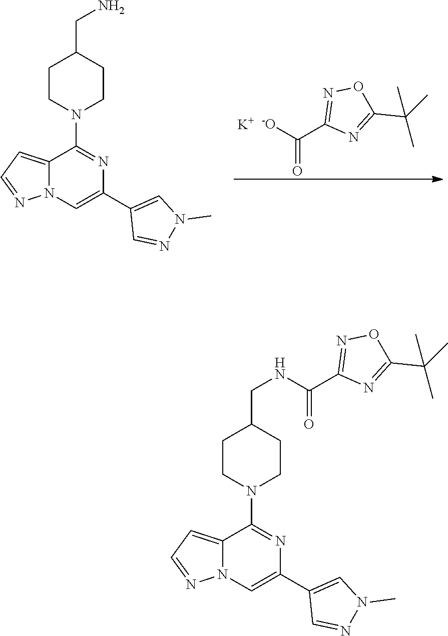

5-(tert-Butyl)-N-((1-(6-(1-methyl-1H-pyrazol-4-yl)pyrazolo[1,5-a]pyrazin-4-yl)piperidin-4-yl)methyl)-1,2,4-oxadiazole-3-carboxamide was obtained as a white solid, 20 mg, 44.8% yield, from potassium 5-(tert-butyl)-1,2,4-oxadiazole-3-carboxylate and (1-(6-(1-methyl-1H-pyrazol-4-yl)pyrazolo[1,5-a]pyrazin-4-yl)piperidin-4-yl)methanamine dihydrochloride following the procedure described in Example 115, step 3. LCMS m/z=464.3 [M+H]⁺ 1H NMR (500 MHz, DMSO-d₆) δ ppm 9.00 (t, J=5.80 Hz, 1H) 8.45 (s, 1H) 8.14 (s, 1H) 7.98-7.88 (m, 2H) 6.92 (d, J=2.44 Hz, 1H) 4.54 (br d, J=13.43 Hz, 2H) 3.87 (s, 3H) 3.21 (t, J=6.41 Hz, 2H) 3.07 (br t, J=11.90 Hz, 2H) 1.99-1.90 (m, 1H) 1.80 (br d, J=10.99 Hz, 2H) 1.42 (s, 9H) 1.36-1.24 (m, 2H).

### Example 137. N-((1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

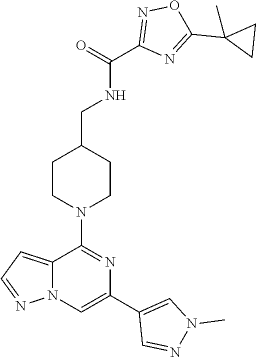 [SMILES: Cn1cc(-c2cn3nccc3c(N3CCC(CNC(=O)c4noc(C5(C)CC5)n4)CC3)n2)cn1]

### 1. Preparation of ethyl (E)-2-(hydroxyimino)-2-(1-methylcyclopropane-1-carboxamido)acetate

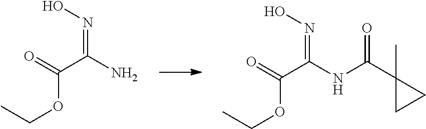

To a solution of ethyl (E)-2-amino-2-(hydroxyimino)acetate (2.5 g, 18.92 mmol) in DCM/DMF (v/v 20/1, 210 mL) was added 1-methylcyclopropanecarboxylic acid (2.08 g, 20.81 mmol), HATU (7.94 g, 20.81 mmol) and DIPEA (7.34 g, 56.76 mmol) and the reaction was stirred at 25° C. for 1 h. The mixture was poured into water (300 mL), extracted with DCM (100 mL×5) and the combined organic layers were evaporated under reduced pressure to afford ethyl (E)-2-(hydroxyimino)-2-(1-methylcyclopropane-1-carboxamido)acetate as a yellow oil, that was used directly in the next step.

### 2. Preparation of ethyl 5-(1-methylcyclopropyl)-1,2,4-oxadiazole-3-carboxylate

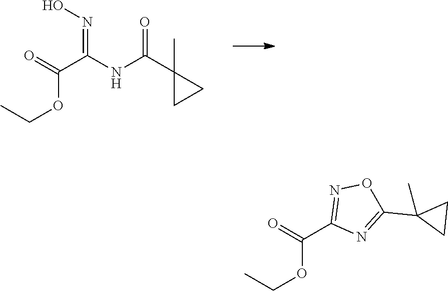

A solution of ethyl (E)-2-(hydroxyimino)-2-(1-methylcyclopropane-1-carboxamido)acetate (5.30 g, 17.15 mmol) in pyridine (100 mL) was stirred at 100° C. for 17 h. The reaction solution was concentrated in vacuo, the residue was poured into HCl solution (1.0 M, 200 mL) and extracted with DCM (100 mL×3). The combined organic layers were concentrated to give the crude, which was purified by column chromatography (PE/EtOAc=30/1 to 10/1) to give ethyl 5-(1-methylcyclopropyl)-1,2,4-oxadiazole-3-carboxylate (1.40 g, 39.7% yield) as colorless oil.

### 3. Preparation of 5-(1-methylcyclopropyl)-1,2,4-oxadiazole-3-carboxylic acid

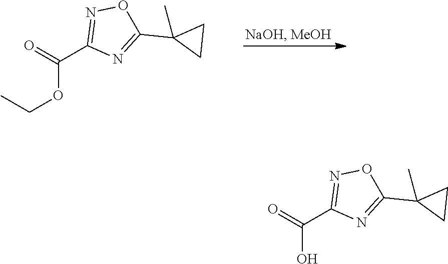

To a solution of ethyl 5-(1-methylcyclopropyl)-1,2,4-oxadiazole-3-carboxylate (1.00 g, 4.86 mmol) in MeOH/H₂O (v/v=10/1, 44 mL) was added NaOH (213.84 mg, 5.35 mmol) and the mixture was stirred at 25° C. for 2 h. The mixture was acidified to pH=6-7 with 2M HCl and then concentrated in vacuo. The crude product was freeze-dried to give 5-(1-methylcyclopropyl)-1,2,4-oxadiazole-3-carboxylic acid (740 mg, crude) as white solid, which was used for the next step directly.

### 4. Preparation of 5-(1-methylcyclopropyl)-1,2,4-oxadiazole-3-carbonyl chloride

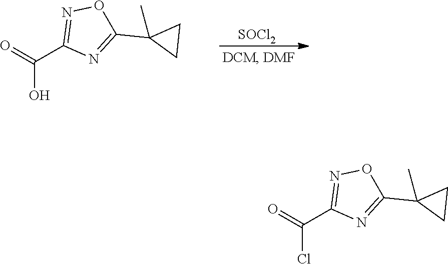

To a solution of 5-(1-methylcyclopropyl)-1,2,4-oxadiazole-3-carboxylic acid (100 mg, 594.7 μmol) in DCM (30 mL) was added DMF (1.0 mL). SOCl₂ (353.8 mg, 2.97 mmol) was added dropwise and the mixture was stirred at 25° C. for 1 h. The mixture was concentrated to give 5-(1-methylcyclopropyl)-1,2,4-oxadiazole-3-carbonyl chloride (120 mg, crude), which was used for the next step directly.

### 5. Preparation of N-((1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

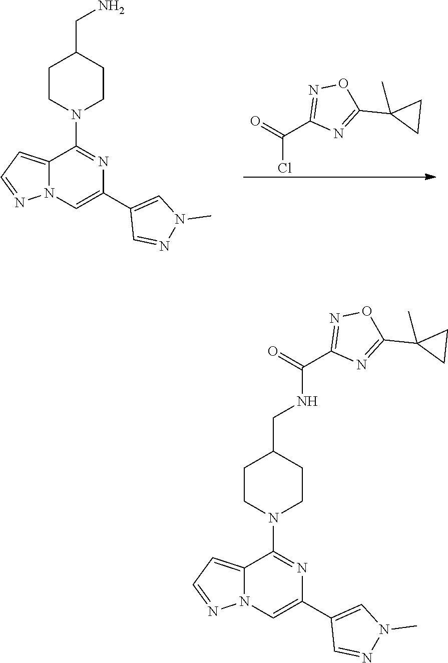 [SMILES: Cn1cc(-c2cn3nccc3c(N3CCC(CN)CC3)n2)cn1 ; CC1(c2nc(C(=O)Cl)no2)CC1 ; Cn1cc(-c2cn3nccc3c(N3CCC(CNC(=O)c4noc(C5(C)CC5)n4)CC3)n2)cn1]

To a mixture of (1-(6-(1-methyl-1H-pyrazol-4-yl)pyrazolo[1,5-a]pyrazin-4-yl)piperidin-4-yl)methanamine dihydrochloride (Example 136, step 2, 80 mg, 230 μmol) in DCM (50 mL) was added DIPEA (89.17 mg, 690 μmol), then 5-(1-methylcyclopropyl)-1,2,4-oxadiazole-3-carbonyl chloride (85.83 mg, 460 μmol) and the reaction was stirred at 20° C. for 1 h. The reaction was concentrated in vacuum and the crude product was purified by prep-HPLC (Method E, 35-65% gradient) to give N-((1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide (48.0 mg, 45.2% yield) as a white solid. LCMS m/z=462.2 [M+H]^{+ 1}HNMR: (400 MHz, DMSO-d₆) δ: 8.91 (t, J=6.0 Hz, 1H), 8.41 (d, J=0.8 Hz, 1H), 8.10 (s, 1H), 7.94-7.87 (m, 2H), 6.91-6.84 (m, 1H), 4.50 (d, J=13.2 Hz, 2H), 3.83 (s, 3H), 3.16 (t, J=6.4 Hz, 2H), 3.03 (t, J=12.0 Hz, 2H), 1.96-1.82 (m, 1H), 1.76 (d, J=10.8 Hz, 2H), 1.50 (s, 3H), 1.36-1.31 (m, 2H), 1.30-1.19 (m, 2H), 1.15-1.10 (m, 2H).

### Example 138. 1-(tert-butyl)-N-((1-(6-(1-methyl-1H-pyrazol-4-yl)pyrazolo[1,5-a]pyrazin-4-yl)piperidin-4-yl)methyl)-1H-1,2,3-triazole-4-carboxamide

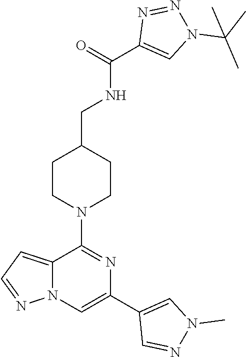 [SMILES: Cn1cc(-c2cn3nccc3c(N3CCC(CNC(=O)c4cn(C(C)(C)C)nn4)CC3)n2)cn1]

To a mixture of (1-(6-(1-methyl-1H-pyrazol-4-yl)pyrazolo[1,5-a]pyrazin-4-yl)piperidin-4-yl)methanamine dihydrochloride (Example 136, step 2, 80.0 mg, 230 μmol) in DCM (50 mL) was added DIPEA (89.17 mg, 690 μmol), 1-tert-butyltriazole-4-carboxylic acid (77.82 mg, 460 μmol) and HATU (96.45 mg, 253 μmol) and the reaction was stirred at 20° C. for 1 h. The reaction was concentrated in vacuum and the crude was purified by prep-HPLC to give 1-(tert-butyl)-N-((1-(6-(1-methyl-1H-pyrazol-4-yl)pyrazolo[1,5-a]pyrazin-4-yl)piperidin-4-yl)methyl)-1H-1,2,3-triazole-4-carboxamide (47.3 mg, 44.5% yield) as a white solid. LCMS m/z=463.1 [M+H]⁺ 1H NMR: (400 MHz, DMSO-d₆) δ: 8.61 (s, 1H), 8.51 (t, J=6.0 Hz, 1H), 8.41 (s, 1H), 8.10 (s, 1H), 7.93-7.88 (m, 2H), 6.87 (d, J=1.6 Hz, 1H), 4.50 (d, J=13.2 Hz, 2H), 3.83 (s, 3H), 3.17 (t, J=6.4 Hz, 2H), 3.03 (t, J=11.6 Hz, 2H), 1.93-1.90 (m, 1H), 1.77 (d, J=12.4 Hz, 2H), 1.59 (s, 9H), 1.34-1.18 (m, 2H).

### Example 139: N-((2-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

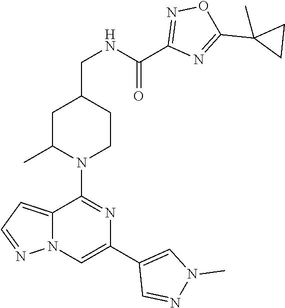 [SMILES: CC1CC(CNC(=O)c2noc(C3(C)CC3)n2)CCN1c1nc(-c2cnn(C)c2)cn2nccc12]

### 1. Preparation of tert-butyl 2-methyl-4-((tosyloxy)methyl)piperidine-1-carboxylate

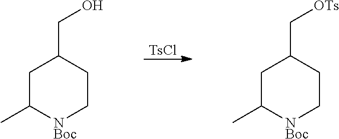

To a solution of tert-butyl 4-(hydroxymethyl)-2-methylpiperidine-1-carboxylate (3.50 g, 15.26 mmol) and TEA (7.72 g, 76.3 mmol) in DCM (150 mL) was slowly added TsCl (5.82 g, 30.52 mmol) followed by DMAP (372.86 mg, 3.05 mmol) and the reaction mixture was stirred at 20° C. for 12 h. The reaction mixture was concentrated in vacuo and the crude was purified by column chromatography (PE/EtOAc=4/1) to give tert-butyl 2-methyl-4-((tosyloxy)methyl)piperidine-1-carboxylate (5.0 g, crude) as clear oil. LCMS m/z=384.0 [M+H]⁺

### 2. Preparation of tert-butyl 4-((1,3-dioxoisoindolin-2-yl)methyl)-2-methylpiperidine-1-carboxylate

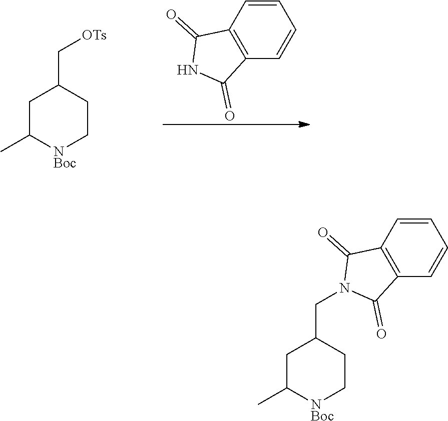 [SMILES: Cc1ccc(S(=O)(=O)OCC2CCN(C(=O)OC(C)(C)C)C(C)C2)cc1 ; O=C1NC(=O)c2ccccc21 ; CC1CC(CN2C(=O)c3ccccc3C2=O)CCN1C(=O)OC(C)(C)C]

To a solution of tert-butyl 2-methyl-4-((tosyloxy)methyl)piperidine-1-carboxylate (5.0 g, 13.04 mmol) and isoindoline-1,3-dione (3.84 g, 26.08 mmol) in DMF (100 mL) was added K₂CO₃ (3.60 g, 26.08 mmol) and the reaction mixture was stirred at 90° C. for 12 h. The mixture was concentrated under vacuum to remove THF and the crude was poured into saturated K₂CO₃ (100 mL) and extracted with EtOAc (100 mL×2). The combined organic layers were washed with water (100 mL), dried over Na₂SO₄, filtered and concentrated under vacuum. The crude was purified by column chromatography on silica gel (PE/EtOAc=4/1) to give tert-butyl 4-((1,3-dioxoisoindolin-2-yl)methyl)-2-methylpiperidine-1-carboxylate (5.0 g, crude) as a white solid. LCMS m/z=359.1 [M+H]⁺.

### 3. Preparation of 2-((2-methylpiperidin-4-yl)methyl)isoindoline-1,3-dione trifluoroacetate

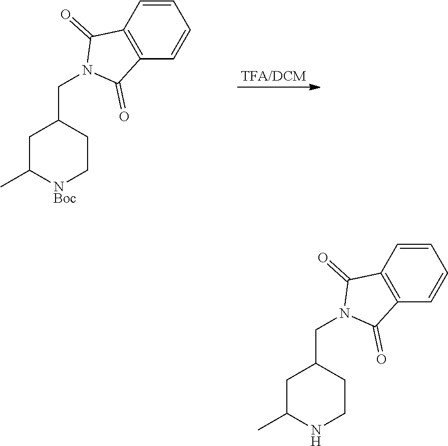 [SMILES: CC1CC(CN2C(=O)c3ccccc3C2=O)CCN1C(=O)OC(C)(C)C ; CC1CC(CN2C(=O)c3ccccc3C2=O)CCN1]

To a solution of tert-butyl 4-((1,3-dioxoisoindolin-2-yl)methyl)-2-methylpiperidine-1-carboxylate (1.00 g, 2.79 mmol) in DCM (20 mL) was added TFA (7.45 g, 65.34 mmol) and the mixture was stirred at 20° C. for 2 h. The mixture was concentrated under vacuum to give 2-((2-methylpiperidin-4-yl)methyl)isoindoline-1,3-dione trifluoroacetate (700 mg, crude) as clear oil, which was used without further purification. LCMS m/z=259.0 [M+H]⁺

### 4. Preparation of 2-((2-methyl-1-(6-(1-methyl-1H-pyrazol-4-yl)pyrazolo[1,5-a]pyrazin-4-yl)piperidin-4-yl)methyl)isoindoline-1,3-dione

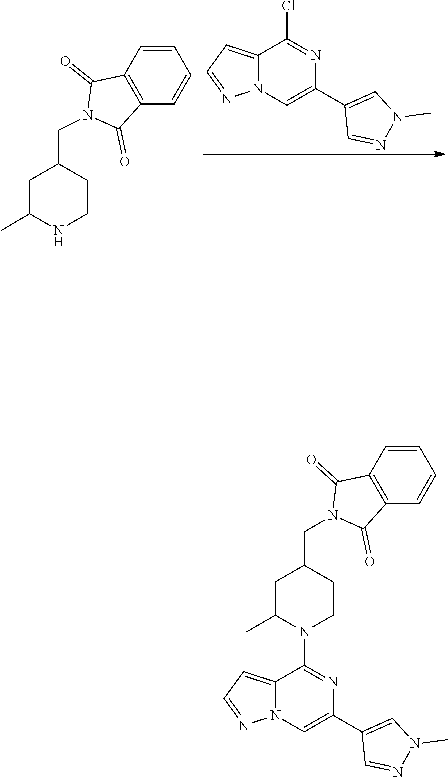

To a solution of 2-((2-methylpiperidin-4-yl)methyl)isoindoline-1,3-dione trifluoroacetate (250.0 mg, 967.8 μmol) in NMP (2 mL) was added DIPEA (375.25 mg, 2.90 mmol), then 4-chloro-6-(1-methyl-1H-pyrazol-4-yl)pyrazolo[1,5-a]pyrazine (Example 1, step 5, 226.14 mg, 967.8 μmol) and the reaction was stirred under microwave irradiation at 180° C. for 2 h. The mixture was poured into H₂O (100 mL) and extracted with EtOAc (100 mL×2). The combined organic layers were washed with brine (100 mL), dried over Na₂SO₄, filtered and concentrated under vacuum. The crude product was purified by column chromatography on silica gel (PE/EtOAc=1/9) to give 2-((2-methyl-1-(6-(1-methyl-1H-pyrazol-4-yl)pyrazolo[1,5-a]pyrazin-4-yl)piperidin-4-yl)methyl)isoindoline-1,3-dione (260 mg, 53.1% yield) as brown oil.

### 5. Preparation of(2-methyl-1-(6-(1-methyl-1H-pyrazol-4-yl)pyrazolo[1,5-a]pyrazin-4-yl)piperidin-4-yl)methanamine dihydrochloride

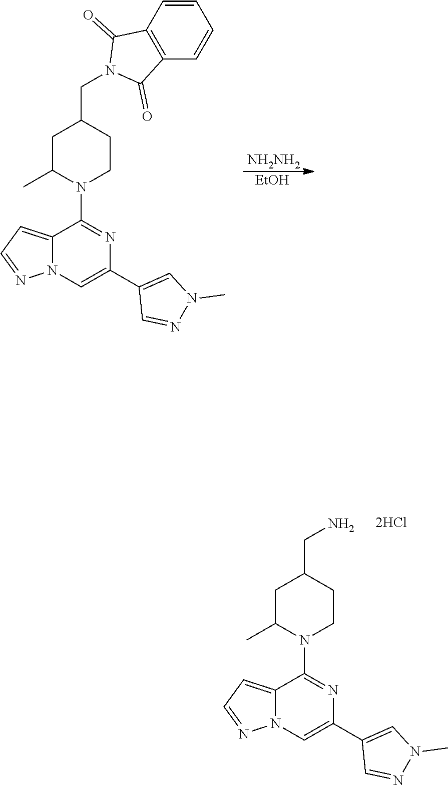 [SMILES: "EtOH" ; CC1CC(CN2C(=O)c3ccccc3C2=O)CCN1c1nc(-c2cnn(C)c2)cn2nccc12 ; "NH2NH2" ; "2HCl" ; CC1CC(CN)CCN1c1nc(-c2cnn(C)c2)cn2nccc12]

To a solution of 2-((2-methyl-1-(6-(1-methyl-1H-pyrazol-4-yl)pyrazolo[1,5-a]pyrazin-4-yl)piperidin-4-yl)methyl)isoindoline-1,3-dione (260 mg, 570.8 μmol) in EtOH (20 mL) was added NH₂NH₂·H₂O (1.00 g, 19.98 mmol) and the reaction was stirred at 50° C. for 5 h. The mixture was concentrated under vacuum to give (2-methyl-1-(6-(1-methyl-1H-pyrazol-4-yl)pyrazolo[1,5-a]pyrazin-4-yl)piperidin-4-yl)methanamine (180 mg, crude) as a brown solid.

To a solution of this compound (180 mg, 553.15 μmol) and TEA (167.92 mg, 1.66 mmol) in DCM (30 mL) was added tert-butoxycarbonyl tert-butyl carbonate (241.45 mg, 1.11 mmol) and the reaction mixture was stirred at 20° C. for 3 h. The reaction mixture was concentrated under vacuum and the crude was purified by column chromatography (PE/EtOAc=1/4) to give tert-butyl ((2-methyl-1-(6-(1-methyl-1H-pyrazol-4-yl)pyrazolo[1,5-a]pyrazin-4-yl)piperidin-4-yl)methyl)carbamate (130 mg, 55.2% yield) as a brown solid.

A solution of this solid (130 mg, 305.5 μmol) in HCl/EtOAc (4 M, 10.0 mL) was stirred at 20° C. for 1 h. The mixture was concentrated under vacuum to give 2-methyl-1-(6-(1-methyl-1H-pyrazol-4-yl)pyrazolo[1,5-a]pyrazin-4-yl)piperidin-4-yl)methanamine dihydrochloride (120 mg, crude) as a brown solid. LCMS m/z=326.1 [M+H]⁺

### 6. Preparation of N-((2-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

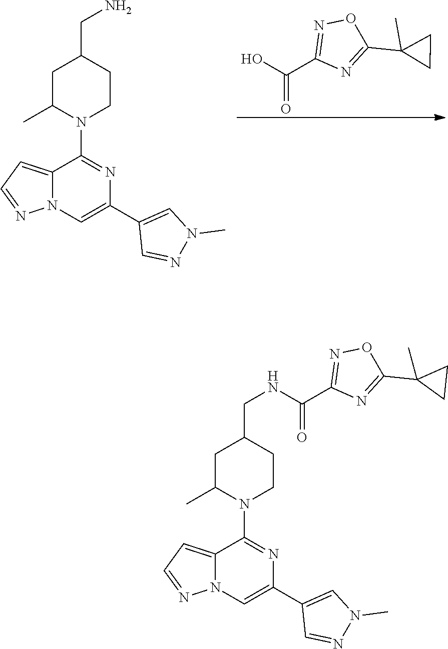 [SMILES: CC1CC(CN)CCN1c1nc(-c2cnn(C)c2)cn2nccc12 ; CC1(c2nc(C(=O)O)no2)CC1 ; CC1CC(CNC(=O)c2noc(C3(C)CC3)n2)CCN1c1nc(-c2cnn(C)c2)cn2nccc12]

N-((2-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide was obtained as a yellow solid, 46 mg, 65% yield, from (2-methyl-1-(6-(1-methyl-1H-pyrazol-4-yl)pyrazolo[1,5-a]pyrazin-4-yl)piperidin-4-yl)methanamine dihydrochloride and 5-(1-methylcyclopropyl)-1,2,4-oxadiazole-3-carboxylic acid (Example 137, step 3) following the procedure described in Example 108.

LCMS m/z=476.1 [M+H]⁺1H NMR: (400 MHz, DMSO-d₆) δ: 8.95-8.89 (m, 1H), 8.45-8.42 (m, 1H), 8.12 (s, 1H), 7.94 (s, 2H), 6.90-6.84 (m, 1H), 4.99-4.20 (m, 2H), 3.86 (s, 3H), 3.57-3.14 (m, 3H), 2.14-1.66 (m, 3H), 1.52 (d, J=4.0 Hz, 3H), 1.41-1.32 (m, 5H), 1.26-1.13 (m, 4H).

### Example 140, 141, 142 and 143. 1-(tert-butyl)-N-(((2S,4R)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide, 1-(tert-butyl)-N-(((2R,4R)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide, 1-(tert-butyl)-N-(((2S,4S)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide and 1-(tert-butyl)-N-(((2R,4S)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide

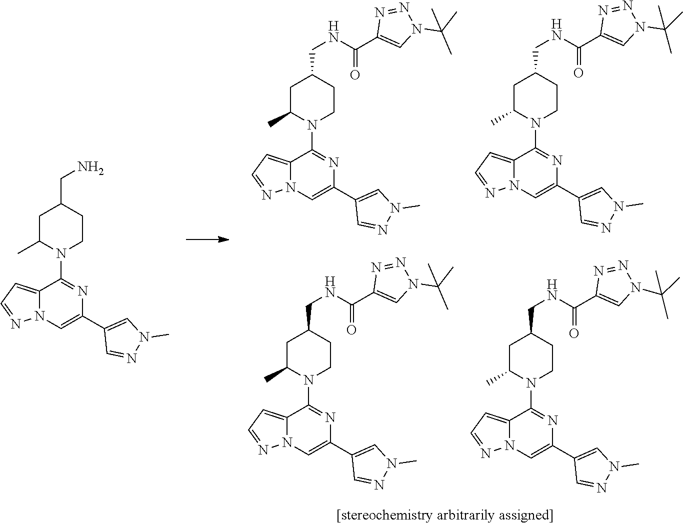 [SMILES: CC1CC(CN)CCN1c1nc(-c2cnn(C)c2)cn2nccc12 ; C[C@H]1C[C@@H](CNC(=O)c2cn(C(C)(C)C)nn2)CCN1c1nc(-c2cnn(C)c2)cn2nccc12 ; C[C@H]1C[C@H](CNC(=O)c2cn(C(C)(C)C)nn2)CCN1c1nc(-c2cnn(C)c2)cn2nccc12 ; C[C@@H]1C[C@@H](CNC(=O)c2cn(C(C)(C)C)nn2)CCN1c1nc(-c2cnn(C)c2)cn2nccc12 ; C[C@@H]1C[C@H](CNC(=O)c2cn(C(C)(C)C)nn2)CCN1c1nc(-c2cnn(C)c2)cn2nccc12]

To a solution of (2-methyl-1-(6-(1-methyl-1H-pyrazol-4-yl)pyrazolo[1,5-a]pyrazin-4-yl)piperidin-4-yl)methanamine dihydrochloride (Example 139, step 5, 150 mg, 414.5 μmol) in DCM (100 mL) was added DIPEA (160.71 mg, 1.24 mmol), 1-(tert-butyl)-1H-1,2,3-triazole-4-carboxylic acid (105.18 mg, 621.8 μmol) and HATU (158.04 mg, 414.51 μmol) and the reaction was stirred at 20° C. for 2 h. The mixture was concentrated under vacuum to give a brown crude, which was purified by Prep-HPLC (Method D, 38-58% gradient) to give 1-(tert-butyl)-N-((2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide (150 mg, crude) as a yellow solid. LCMS m/z=477.2 [M+H]⁺

This solid was further purified by SFC (Column: DAICEL CHIRALPAK AD (250 mm×30 mm, 10 μm), 0.1% NH₃·H₂O EtOH, Flow Rate (mL/min): 80, Column temp.: 35° C. to give:

1-(tert-butyl)-N-(((2S,4R)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide (stereochemistry arbitrarily assigned) (26 mg, 17.3% yield) as a white solid, LCMS m/z=477.1 [M+H]⁺. 1HNMR (400 MHz, DMSO-d₆) δ=8.64 (s, 1H), 8.54 (t, J=6.0 Hz, 1H), 8.41 (s, 1H), 8.12 (s, 1H), 7.92 (d, J=4.0 Hz, 2H), 6.87 (s, 1H), 4.98 (t, J=6.0 Hz, 1H), 4.44 (d, J=14.0 Hz, 1H), 3.86 (s, 3H), 3.21-3.16 (m, 3H), 2.15 (s, 1H), 1.84-1.67 (m, 2H), 1.63 (s, 9H), 1.49-1.41 (in, 1H), 1.27-1.24 (in, 4H).

1-(tert-butyl)-N-(((2R,4R)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide (stereochemistry arbitrarily assigned) (18.50 mg, 12.3% yield) as a white solid, LCMS m/z=477.1 [M+H]^{+ 1}HNMR (400 MHz, DMSO-d₆) δ: 8.62 (s, 1H), 8.52 (t, J=6.0 Hz, 1H), 8.44 (s, 1H), 8.11 (s, 1H), 7.93-7.92 (m, 2H), 6.83 (s, 1H), 4.24-4.20 (m, 1H), 3.93-3.89 (m, 4H), 3.56-3.49 (m, 1H), 3.29-3.18 (m, 2H), 1.98-1.77 (m, 3H), 1.61 (s, 9H), 1.41-1.31 (m, 5H)

1-(tert-butyl)-N-(((2S,4S)-2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide (stereochemistry arbitrarily assigned) (21 mg, 14% yield) as a white solid, LCMS m/z=477.1 [M+H]^{+ 1}HNMR (400 MHz, DMSO-d₆) δ: 8.62 (s, 1H), 8.53 (t, J=6.0 Hz, 1H), 8.45 (s, 1H), 8.11 (s, 1H), 7.94-7.92 (m, 2H), 6.83 (s, 1H), 4.25-4.20 (m, 1H), 3.93-3.89 (m, 1H), 3.86 (s, 3H), 3.56-3.49 (m, 1H), 3.27-3.18 (m, 2H), 1.98-1.89 (m, 2H), 1.83-1.77 (m, 1H), 1.61 (s, 9H), 1.41-1.35 (m, 2H), 1.34-1.31 (m, 3H).

And 1-(tert-butyl)-N-((2-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide(stereochemistry arbitrarily assigned), (17.0 mg, 11.3% yield) as a white solid. LCMS m/z=477.1 [M+H]^{+ 1}HNMR (400 MHz, DMSO-d₆) δ: 8.65 (s, 1H), 8.54 (t, J=6.0 Hz, 1H), 8.41 (s, 1H), 8.12 (s, 1H), 7.94-7.92 (m, 2H), 6.88 (s, 1H), 4.98 (t, J=6.0 Hz, 1H), 4.45 (d, J=14.0 Hz, 1H), 3.86 (s, 3H), 3.21-3.16 (m, 3H), 2.15 (s, 1H), 1.84-1.67 (m, 2H), 1.63 (s, 9H), 1.47-1.41 (m, 1H), 1.27-1.24 (m, 4H).

### Example 144: 5-(tert-butyl)-N-((3-methyl-1-(6-(1-methyl-1H-pyrazol-4-yl)pyrazolo[1,5-a]pyrazin-4-yl)piperidin-4-yl)methyl)-1,2,4-oxadiazole-3-carboxamide

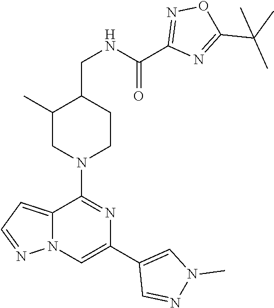 [SMILES: CC1CN(c2nc(-c3cnn(C)c3)cn3nccc23)CCC1CNC(=O)c1noc(C(C)(C)C)n1]

### 1. Preparation of tert-butyl ((3-methyl-1-(6-(1-methyl-1H-pyrazol-4-yl)pyrazolo[1,5-a]pyrazin-4-yl)piperidin-4-yl)methyl)carbamate

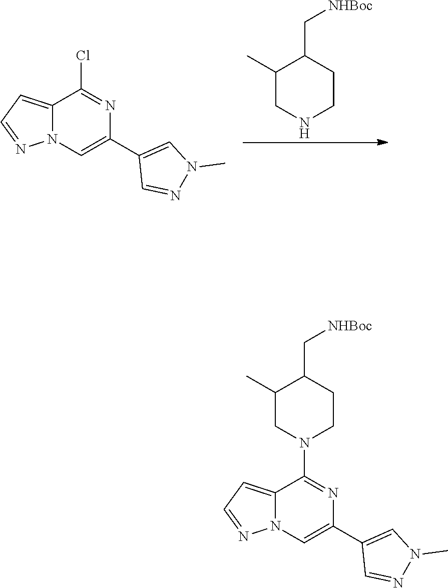 [SMILES: Cn1cc(-c2cn3nccc3c(Cl)n2)cn1 ; CC1CNCCC1CNC(=O)OC(C)(C)C ; CC1CN(c2nc(-c3cnn(C)c3)cn3nccc23)CCC1CNC(=O)OC(C)(C)C]

tert-Butyl ((3-methyl-1-(6-(1-methyl-1H-pyrazol-4-yl)pyrazolo[1,5-a]pyrazin-4-yl)piperidin-4-yl)methyl)carbamate was obtained, from tert-butyl ((3-methylpiperidin-4-yl)methyl)carbamate and 4-chloro-6-(1-methylpyrazol-4-yl)pyrazolo[1,5-a]pyrazine (Example 1, step 5) following the procedure described in Example 136, step 1. LCMS m/z=426.2 [M+H]⁺

### 2. Preparation of(3-methyl-1-(6-(1-methyl-1H-pyrazol-4-yl)pyrazolo[1,5-a]pyrazin-4-yl)piperidin-4-yl)methanamine dihydrochloride

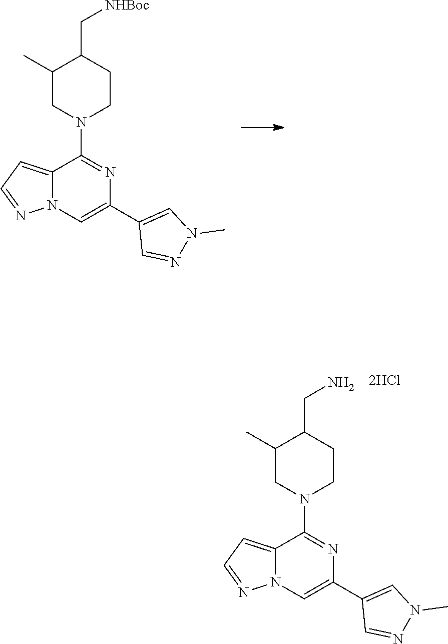 [SMILES: CC1CN(c2nc(-c3cnn(C)c3)cn3nccc23)CCC1CNC(=O)OC(C)(C)C ; CC1CN(c2nc(-c3cnn(C)c3)cn3nccc23)CCC1CN]

(3-Methyl-1-(6-(1-methyl-1H-pyrazol-4-yl)pyrazolo[1,5-a]pyrazin-4-yl)piperidin-4-yl)methanamine dihydrochloride was obtained as a white solid, from tert-butyl ((3-methyl-1-(6-(1-methyl-1H-pyrazol-4-yl)pyrazolo[1,5-a]pyrazin-4-yl)piperidin-4-yl)methyl)carbamate following the procedure described in Example 115, step 2. LCMS m/z=326.2 [M+H]⁺

### 3. Preparation of 5-(tert-butyl)-N-((3-methyl-1-(6-(1-methyl-1H-pyrazol-4-yl)pyrazolo[1,5-a]pyrazin-4-yl)piperidin-4-yl)methyl)-1,2,4-oxadiazole-3-carboxamide

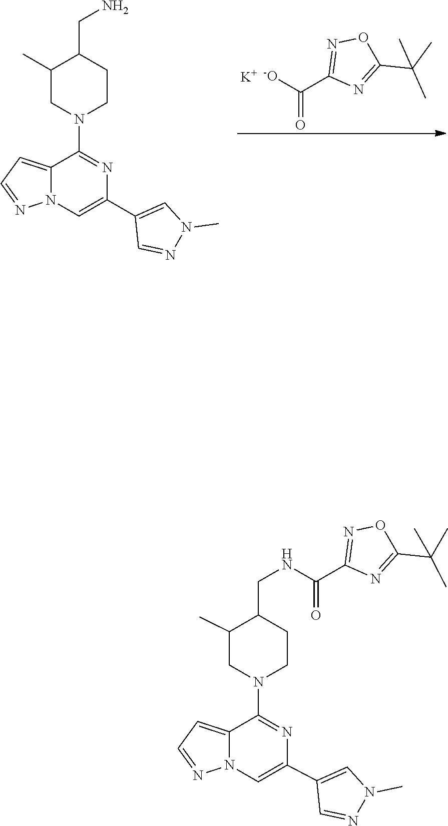

5-(tert-Butyl)-N-((3-methyl-1-(6-(1-methyl-1H-pyrazol-4-yl)pyrazolo[1,5-a]pyrazin-4-yl)piperidin-4-yl)methyl)-1,2,4-oxadiazole-3-carboxamide was obtained as a white solid, 11 mg, 13% yield from (3-methyl-1-(6-(1-methyl-1H-pyrazol-4-yl)pyrazolo[1,5-a]pyrazin-4-yl)piperidin-4-yl)methanamine dihydrochloride and potassium 5-tert-butyl-1,2,4-oxadiazole-3-carboxylate, following the procedure described in Example 136, step 3. LCMS m/z=478.2 [M+H]⁺. ¹H NMR (500 MHz, DMSO-d₆) δ ppm 9.01-8.95 (m, 1H) 8.91 (t, J=6.41 Hz, 1H) 8.44 (s, 1H) 8.17-8.11 (m, 1H) 7.96-7.90 (m, 2H) 6.91 (d, J=1.83 Hz, 1H) 4.57-4.44 (m, 1H) 4.23-4.26 (m, 1H) 3.87 (s, 3H) 3.50 (dt, J=13.73, 4.43 Hz, 1H) 3.30-3.23 (m, 1H) 3.22-3.14 (m, 1H) 3.13-3.01 (m, 1H) 2.85-2.76 (m, 1H) 2.42 (br s, 1H) 2.17-1.99 (m, 1H) 1.81 (br d, J=10.38 Hz, 1H) 1.67-1.47 (m, 3H) 1.44-1.40 (m, 10H) 1.40-1.30 (m, 1H) 1.28-1.17 (m, 1H) 1.05 (d, J=6.10 Hz, 2H) 0.94 (dd, J=9.16, 6.71 Hz, 2H).

### Examples 145, 146, 147 and 148: N-(((3S,4R)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide, N-(((3S,4S)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide, N-(((3R,4R)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide and N-(((3R,4S)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

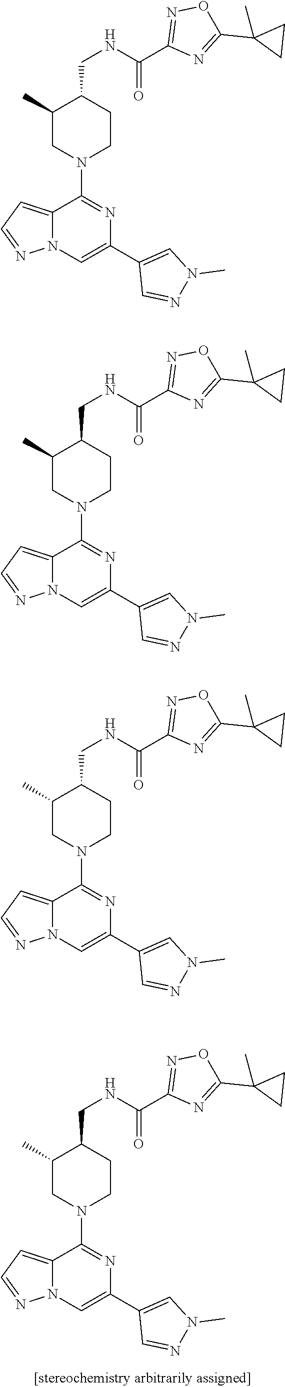 [SMILES: C[C@@H]1CN(c2nc(-c3cnn(C)c3)cn3nccc23)CC[C@H]1CNC(=O)c1noc(C2(C)CC2)n1 ; C[C@@H]1CN(c2nc(-c3cnn(C)c3)cn3nccc23)CC[C@@H]1CNC(=O)c1noc(C2(C)CC2)n1 ; C[C@H]1CN(c2nc(-c3cnn(C)c3)cn3nccc23)CC[C@H]1CNC(=O)c1noc(C2(C)CC2)n1 ; C[C@H]1CN(c2nc(-c3cnn(C)c3)cn3nccc23)CC[C@@H]1CNC(=O)c1noc(C2(C)CC2)n1]

DIPEA (143.08 mg, 1.11 mmol) was added to a solution of (3-methyl-1-(6-(1-methyl-1H-pyrazol-4-yl)pyrazolo[1,5-a]pyrazin-4-yl)piperidin-4-yl)methanamine dihydrochloride (Example 144, step 2, 126 mg, 221.4 μmol) and potassium 5-(1-methylcyclopropyl)-1,2,4-oxadiazole-3-carboxylate (91.78 mg, 442.8 μmol) in DMF (2.21 mL). T3P® (422.7 mg, 664.3 μmol, 50% purity) was added and the reaction was stirred overnight at rt. Additional equivalents of potassium 5-(1-methylcyclopropyl)-1,2,4-oxadiazole-3-carboxylate, DIPEA and T3P® were added and the reaction was stirred overnight at rt. The reaction was diluted with water, extracted with EtOAc (2×), the combined organic layers were washed with brine, dried over Na₂SO₄, filtered, and concentrated in vacuo. The material was purified by column chromatography (gradient elution 0-75% [3:1 EtOAc:EtOH]:Heptane) to afford a clear film. This was further purified by SFC:CHIRALPAK AD-H 30×250 mm, Sum Method: 40% (1:1) MeOH:DCM w/0.1% DEA in C₀₂ (flow rate: 100 mL/min, ABPR 120bar, MBPR 40 psi, column temp 40° C.). The compounds were re-purified by column chromatography (4 g column, gradient elution 0-100% [3:1 EtOAc:EtOH]:Heptane) to afford the products as clear films.

Peak 1, N-(((3S,4R)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide, (4.0 mg, 3.8% yield, 99% purity). LCMS m/z=476.3 [M+H]⁺. ¹H NMR (600 MHz, DMSO-d₆) δ ppm 8.86 (br t, J=5.9 Hz, 1H), 8.44 (s, 1H), 8.13 (s, 1H), 7.96-7.92 (m, 2H), 6.91 (d, J=2.2 Hz, 1H), 4.53 (br d, J=13.2 Hz, 2H), 4.40 (br d, J=11.0 Hz, 2H), 3.87 (s, 3H), 3.48 (dt, J=12.8, 5.0 Hz, 2H), 3.19-3.12 (m, 2H), 3.06 (td, J=12.8, 2.2 Hz, 2H), 2.79 (br dd, J=13.2, 11.0 Hz, 2H), 1.80 (br dd, J=13.6, 3.3 Hz, 2H), 1.63-1.55 (m, 2H), 1.52 (s, 3H), 1.37-1.34 (m, 2H), 1.16-1.12 (m, 2H), 1.05 (d, J=5.9 Hz, 2H)

Peak 2, N-(((3S,4S)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide (2.0 mg, 1.9% yield, 99% purity) LCMS m/z=476.3 [M+H]^{+ 1}H NMR (600 MHz, DMSO-d₆) δ ppm 8.93 (br t, J=5.9 Hz, 1H), 8.44 (s, 1H), 8.14 (s, 1H), 7.99-7.90 (m, 2H), 6.91 (br d, J=2.2 Hz, 1H), 4.47 (br d, J=12.5 Hz, 2H), 4.31 (br d, J=11.7 Hz, 2H), 3.87 (s, 2H), 3.25-3.21 (m, 2H), 3.12-3.05 (m, 2H), 2.10 (br s, 1H), 2.04-1.96 (m, 1H), 1.60-1.56 (m, 2H), 1.54 (s, 3H), 1.41-1.35 (m, 2H), 1.17 (br d, J=2.2 Hz, 1H), 0.92 (br d, J=6.6 Hz, 3H)

Peak 3, N-(((3R,4R)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide (3.0 mg, 2.8% yield, 99% purity) LCMS m/z=476.3 [M+H]^{+ 1}H NMR (600 MHz, DMSO-d₆) δ ppm 8.93 (br t, J=5.9 Hz, 1H), 8.43 (s, 1H), 8.14 (s, 1H), 7.97-7.92 (m, 2H), 6.91 (d, J=1.5 Hz, 1H), 4.47 (br d, J=13.9 Hz, 2H), 4.31 (br d, J=11.7 Hz, 2H), 3.87 (s, 3H), 3.25-3.20 (m, 3H), 3.12-3.06 (m, 2H), 2.14-2.07 (m, 2H), 2.04-1.99 (m, 1H), 1.58 (br dd, J=9.5, 4.4 Hz, 2H), 1.54 (s, 3H), 1.40-1.35 (m, 3H), 1.20-1.13 (m, 4H), 0.92 (d, J=7.3 Hz, 4H)

Peak 4, N-(((3R,4S)-3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide (6.0 mg, 5.6% yield, 99% purity) LCMS m/z=476.3 [M+H]^{+ 1}H NMR (600 MHz, DMSO-d₆) δ ppm 8.86 (br t, J=5.9 Hz, 1H), 8.44 (s, 1H), 8.13 (s, 1H), 7.97-7.92 (m, 2H), 6.91 (d, J=2.2 Hz, 1H), 4.53 (br d, J=13.9 Hz, 2H), 4.45-4.36 (m, 2H), 3.87 (s, 3H), 3.48 (dt, J=13.2, 4.8 Hz, 1H), 3.19-3.12 (m, 2H), 3.10-3.01 (m, 2H), 2.79 (br dd, J=13.2, 10.3 Hz, 2H), 1.80 (br dd, J=13.2, 2.9 Hz, 2H), 1.63-1.56 (m, 2H), 1.52 (s, 4H), 1.38-1.34 (m, 2H), 1.34-1.28 (m, 1H), 1.17-1.13 (m, 2H), 1.05 (d, J=6.6 Hz, 3H)

### Example 149. N-((3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

### 1. Preparation of tert-butyl 3-methyl-4-((5-(1-methylcyclopropyl)-1,2,4-oxadiazole-3-carboxamido)methyl)piperidine-1-carboxylate

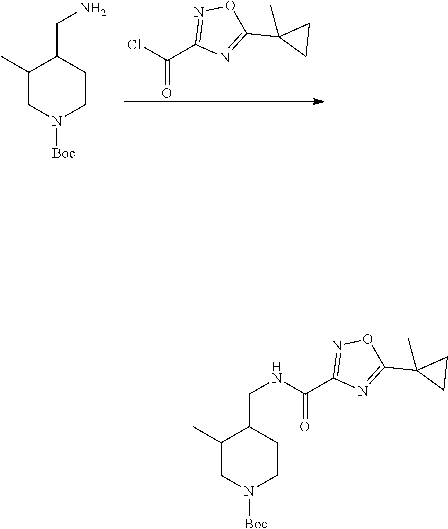 [SMILES: CC1CN(C(=O)OC(C)(C)C)CCC1CN ; CC1(c2nc(C(=O)Cl)no2)CC1 ; CC1CN(C(=O)OC(C)(C)C)CCC1CNC(=O)c1noc(C2(C)CC2)n1]

To a solution of tert-butyl 4-(aminomethyl)-3-methylpiperidine-1-carboxylate (100 mg, 438 μmol) in DCM (20 mL) was added DIPEA (113.20 mg, 875.9 μmol) and 5-(1-methylcyclopropyl)-1,2,4-oxadiazole-3-carbonyl chloride (Example 137, step 4, 163.45 mg, 875.9 μmol) and the reaction was stirred at 20° C. for 1 h. The mixture was poured into water (50 mL) and extracted with DCM (50 mL×3). The combined organic layers were washed with brine (100 mL), dried over Na₂SO₄, filtered and concentrated under vacuum. The crude was purified by column chromatography on silica gel (PE/EtOAc=1/1) to give tert-butyl 3-methyl-4-((5-(1-methylcyclopropyl)-1,2,4-oxadiazole-3-carboxamido)methyl)piperidine-1-carboxylate (120 mg, 72.4% yield) as a yellow solid. LCMS m/z=401.1 [M+Na]+

### 2. Preparation of 5-(1-methylcyclopropyl)-N-((3-methylpiperidin-4-yl)methyl)-1,2,4-oxadiazole-3-carboxamide

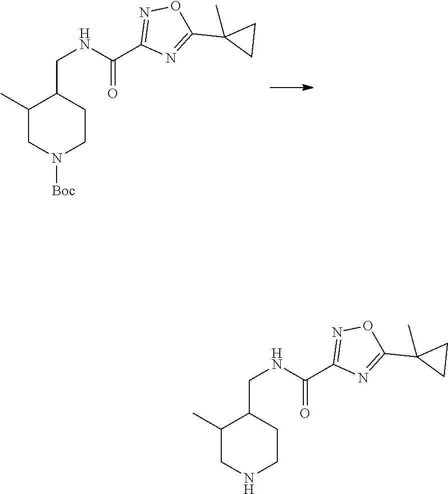 [SMILES: CC1CN(C(=O)OC(C)(C)C)CCC1CNC(=O)c1noc(C2(C)CC2)n1 ; CC1CNCCC1CNC(=O)c1noc(C2(C)CC2)n1]

A solution of tert-butyl 3-methyl-4-((5-(1-methylcyclopropyl)-1,2,4-oxadiazole-3-carboxamido)methyl)piperidine-1-carboxylate (120 mg, 317.1 μmol) in HCl/EtOAc (20 mL) was stirred at 20° C. for 1 h. The mixture was concentrated under vacuum to give 5-(1-methylcyclopropyl)-N-((3-methylpiperidin-4-yl)methyl)-1,2,4-oxadiazole-3-carboxamide hydrochloride (60 mg, crude) as a yellow solid, which was used without further purification. LCMS m/z=279.0 [M+H]⁺

### 3. Preparation of N-((3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

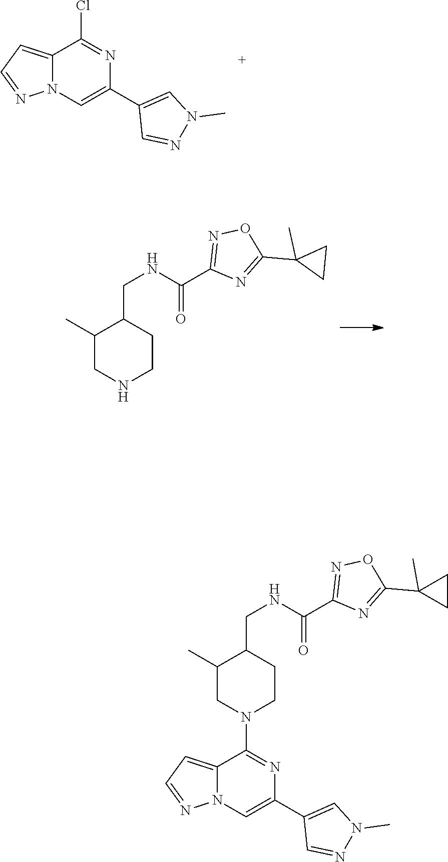 [SMILES: Cn1cc(-c2cn3nccc3c(Cl)n2)cn1 ; CC1CNCCC1CNC(=O)c1noc(C2(C)CC2)n1 ; CC1CN(c2nc(-c3cnn(C)c3)cn3nccc23)CCC1CNC(=O)c1noc(C2(C)CC2)n1]

To a solution of 5-(1-methylcyclopropyl)-N-((3-methylpiperidin-4-yl)methyl)-1,2,4-oxadiazole-3-carboxamide hydrochloride (50 mg, 158.8 μmol) in IPA (10 mL) was added DIPEA (61.58 mg, 476.5 μmol) at 20° C. 4-Chloro-6-(1-methyl-1H-pyrazol-4-yl)pyrazolo[1,5-a]pyrazine (Example 1, step 5, 74.22 mg, 317.7 μmol) was added and the mixture was stirred at 90° C. for 48 h. The reaction was concentrated under vacuum, the mixture was poured into water (20 mL) and extracted with DCM (20 mL×3). The combined organic layers were washed with brine (10 mL), dried over Na₂SO₄, filtered and concentrated under vacuum. The crude was purified by Prep-HPLC (Method E, 38-65% gradient) to give N-((3-methyl-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide (9.10 mg, 12.1% yield) as a white solid. LCMS m/z=476.1 [M+H]^{+ 1}H NMR (400 MHz, DMSO-d₆) δ: 8.95-8.90 (m, 1H), 8.43 (s, 1H), 8.14 (s, 1H), 7.95-7.93 (m, 2H), 6.91 (s, 1H), 4.49-4.29 (m, 2H), 3.87 (s, 3H), 3.28-3.21 (m, 4H), 2.11-2.00 (m, 2H), 1.58-1.51 (m, 5H), 1.39-1.36 (m, 2H), 1.18-1.16 (m, 2H), 0.92 (d, J=6.8 Hz, 3H).

### Examples 150 and 151. 1-(tert-butyl)-N-(((3S,4S)-3-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide and 1-(tert-butyl)-N-(((3R,4R)-3-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide 1. Preparation of cis-2-((3-methylpiperidin-4-yl)methyl)isoindoline-1,3-dione trifluoroacetate

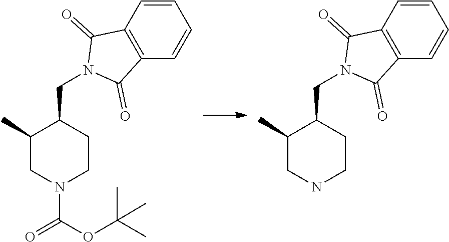 [SMILES: C[C@@H]1CN(C(=O)OC(C)(C)C)CC[C@@H]1CN1C(=O)c2ccccc2C1=O ; C[C@@H]1CNCC[C@@H]1CN1C(=O)c2ccccc2C1=O]

A solution of cis-2-((3-methylpiperidin-4-yl)methyl)isoindoline-1,3-dione (800 mg, 2.23 mmol) in TFA (10 mL) and DCM (40 mL) was stirred at 20° C. for 1 h. The mixture was concentrated under vacuum to give cis-2-((3-methylpiperidin-4-yl)methyl)isoindoline-1,3-dione trifluoroacetate (600 mg, crude) as a yellow solid, which was used without further purification. LCMS m/z=259.0 [M+H]⁺

### 2. Preparation of cis-tert-butyl (4-((1,3-dioxoisoindolin-2-yl)methyl)-3-methylpiperidine-1-carboxylate

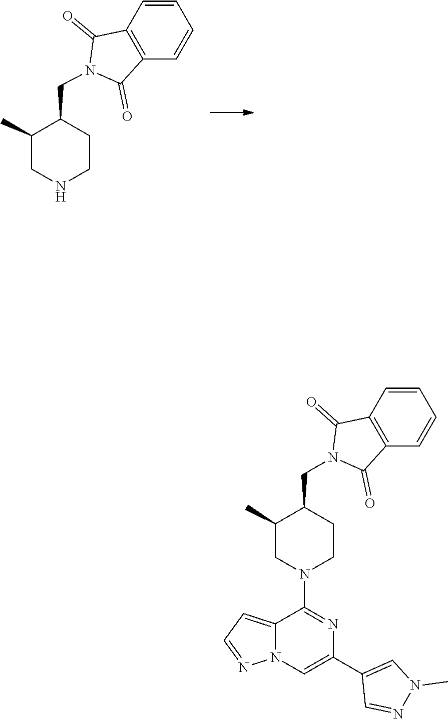 [SMILES: C[C@@H]1CNCC[C@@H]1CN1C(=O)c2ccccc2C1=O ; C[C@@H]1CN(c2nc(-c3cnn(C)c3)cn3nccc23)CC[C@@H]1CN1C(=O)c2ccccc2C1=O]

To a solution of cis-2-((3-methylpiperidin-4-yl)methyl)isoindoline-1,3-dione trifluoroacetate (600 mg, 1.62 mmol) in i-PrOH (20 mL) was added 4-chloro-6-(1-methyl-1H-pyrazol-4-yl)pyrazolo[1,5-a]pyrazine (Example 1, step 5, 300 mg, 1.28 mmol) and DIPEA (497.8 mg, 3.85 mmol) and the reaction was stirred at 90° C. for 48 h. The mixture was filtered and concentrated under vacuum. The crude product was purified by column chromatography on silica gel (PE/EtOAc=1/1 to 0/1) to give cis-tert-butyl (4-((1,3-dioxoisoindolin-2-yl)methyl)-3-methylpiperidine-1-carboxylate (330 mg, 56.6% yield) as a yellow solid. LCMS m/z=456.1 [M+H]⁺

### 3. Preparation of cis-2-((3-methyl-1-(6-(1-methyl-1H-pyrazol-4-yl)pyrazolo[1,5-a]pyrazin-4-yl)piperidin-4-yl)methyl)isoindoline-1,3-dione

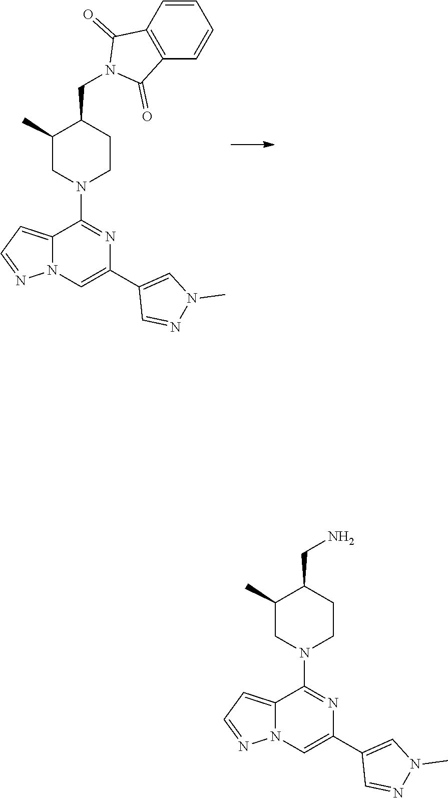 [SMILES: C[C@@H]1CN(c2nc(-c3cnn(C)c3)cn3nccc23)CC[C@@H]1CN1C(=O)c2ccccc2C1=O ; C[C@@H]1CN(c2nc(-c3cnn(C)c3)cn3nccc23)CC[C@@H]1CN]

To a solution of cis-tert-butyl (4-((1,3-dioxoisoindolin-2-yl)methyl)-3-methylpiperidine-1-carboxylate (330 mg, 724.5 μmol) in MeOH (20 mL) was added NH₂NH₂·H₂O (300 mg, 85% purity) and the reaction stirred at 20° C. for 2 h. The mixture was concentrated under vacuum to give cis-2-((3-methyl-1-(6-(1-methyl-1H-pyrazol-4-yl)pyrazolo[1,5-a]pyrazin-4-yl)piperidin-4-yl)methyl)isoindoline-1,3-dione (230 mg, crude) as a yellow solid, which was used directly without further purification. LCMS m/z=326.1 [M+H]⁺

### 4. Preparation of 1-(tert-butyl)-N-(((3S,4S)-3-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide and 1-(tert-butyl)-N-(((3R,4R)-3-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide

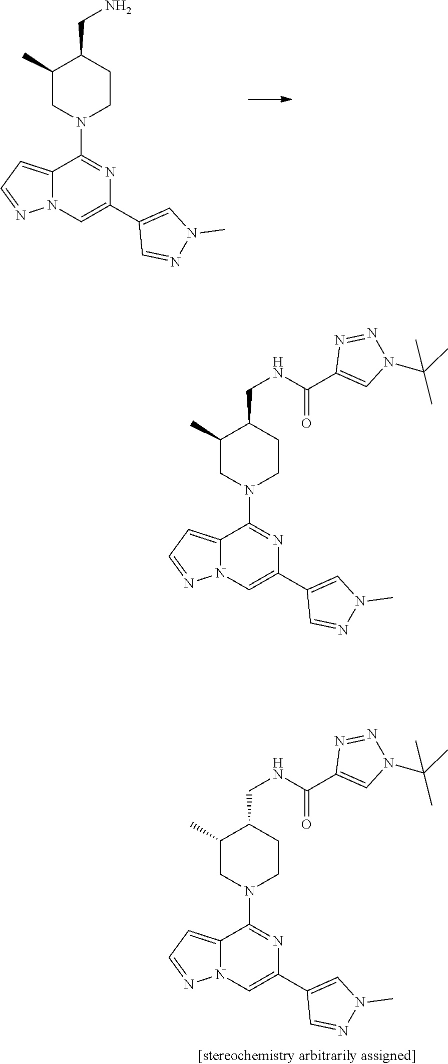 [SMILES: C[C@@H]1CN(c2nc(-c3cnn(C)c3)cn3nccc23)CC[C@@H]1CN ; C[C@@H]1CN(c2nc(-c3cnn(C)c3)cn3nccc23)CC[C@@H]1CNC(=O)c1cn(C(C)(C)C)nn1 ; C[C@H]1CN(c2nc(-c3cnn(C)c3)cn3nccc23)CC[C@H]1CNC(=O)c1cn(C(C)(C)C)nn1]

To a solution of cis-2-((3-methyl-1-(6-(1-methyl-1H-pyrazol-4-yl)pyrazolo[1,5-a]pyrazin-4-yl)piperidin-4-yl)methyl)isoindoline-1,3-dione (190 mg, 433.4 μmol) in DCM (50 mL) was added DIPEA (112.02 mg, 866.7 μmol) at 20° C. 2-(tert-Butyl)-2H-1,2,3-triazole-4-carboxylic acid (109.97 mg, 650.0 μmol) and HATU (247.82 mg, 650.0 μmol) were added and the reaction was stirred at 20° C. for 1 h. The mixture was poured into water (50 mL) and extracted with DCM (50 mL×3). The combined organic layers were washed with brine (100 mL), dried over Na₂SO₄, filtered and concentrated under vacuum. The crude product was purified by Prep-HPLC (Method E, 33-63% gradient) to give cis-(3-methyl-1-(6-(1-methyl-1H-pyrazol-4-yl)pyrazolo[1,5-a]pyrazin-4-yl)piperidin-4-yl)methanamine (180 mg, 85.4% yield) as a yellow solid. LCMS m/z=477.1 [M+H]⁺

This compound (180 mg, 377.7 μmol) was purified by SFC (column: Phenomenex-Cellulose-2 (250 mm×30 mm, 5 μm), 0.1% NH₃·H₂O, EtOH as mobile phase, from 55-55%, Flow Rate (mL/min): 70) to give:

Peak 1, 1-(tert-Butyl)-N-(((3S,4S)-3-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide (stereochemistry arbitrarily assigned) (66.6 mg, 37.0% yield) as a white solid. LCMS m/z=477.2 [M+H]^{+ 1}H NMR (400 MHz, DMSO-d₆) δ: 8.64 (s, 1H), 8.54-8.50 (m, 1H), 8.42 (s, 1H), 8.14-8.12 (in, 1H), 7.95-7.92 (m, 2H), 6.90 (d, J=1.6 Hz, 1H), 4.50-4.30 (m, 2H), 3.87 (s, 3H), 3.27-3.24 (m, 3H), 3.24-3.09 (m, 1H), 2.12-2.01 (m, 2H), 1.63 (s, 9H), 1.62-1.60 (m, 2H), 0.93 (d, J=7.2 Hz, 3H).

and Peak 2, 1-(tert-butyl)-N-(((3R,4R)-3-methyl-1-(6-(1-methyl-1H-pyrazol-4-yl)pyrazolo[1,5-a]pyrazin-4-yl)piperidin-4-yl)methyl)-1H-1,2,3-triazole-4-carboxamide (stereochemistry arbitrarily assigned) (54.0 mg, 30% yield) as a white solid. LCMS m/z 477.1 [M+H]^{+ 1}H NMR (400 MHz, DMSO-d₆) δ: 8.64 (s, 1H), 8.54-8.50 (i, 1H), 8.42 (s, 1H), 8.14-8.12 (m, 1H), 7.95-7.92 (m, 2H), 6.90 (d, J 1.6 Hz, 1H), 4.50-4.30 (i, 2H), 3.87 (s, 3H), 3.27-3.24 (m, 3H), 3.09-3.24 (m, 1H), 2.12-2.01 (m, 2H), 1.63 (s, 9H), 1.62-1.60 (m, 2H), 0.93 (d, J=7.2 Hz, 3H).

### Example 152: 1-(tert-butyl)-N-((3-fluoro-1-(6-(1-methyl-1H-pyrazol-4-yl)pyrazolo[1,5-a]pyrazin-4-yl)piperidin-4-yl)methyl)-1H-1,2,3-triazole-4-carboxamide

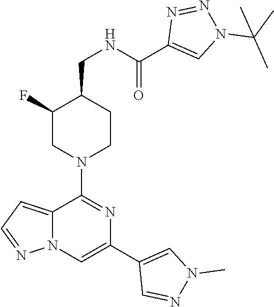 [SMILES: Cn1cc(-c2cn3nccc3c(N3CC[C@H](CNC(=O)c4cn(C(C)(C)C)nn4)[C@H](F)C3)n2)cn1]

### 1. Preparation of benzyl (((3S,4R)-3-fluoro-1-(6-(1-methyl-1H-pyrazol-4-yl)pyrazolo[1,5-a]pyrazin-4-yl)piperidin-4-yl)methyl)carbamate

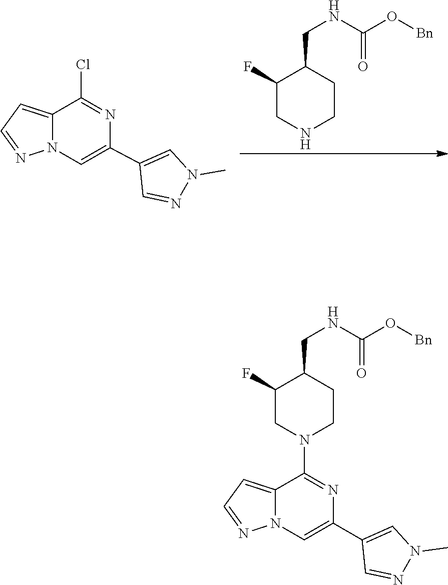 [SMILES: Cn1cc(-c2cn3nccc3c(Cl)n2)cn1 ; O=C(NC[C@H]1CCNC[C@H]1F)OCc1ccccc1 ; Cn1cc(-c2cn3nccc3c(N3CC[C@H](CNC(=O)OCc4ccccc4)[C@H](F)C3)n2)cn1]

A mixture of benzyl (((3S,4R)-3-fluoropiperidin-4-yl)methyl)carbamate hydrochloride (500 mg, 1.65 mmol), 4-chloro-6-(1-methylpyrazol-4-yl)pyrazolo[1,5-a]pyrazine (Example 1, step 5, 367.5 mg, 1.57 mmol) and DIPEA (813.06 mg, 6.29 mmol) were dissolved in DMF (3.30 mL) and the reaction was stirred at 90° C. overnight. The reaction was diluted with water and extracted with EtOAc (2×). The combined organic layers were washed with brine, dried over Na₂SO₄, filtered, and concentrated. The material was triturated with EtOAc and filtered through a pad of Celite® and the solid collected. The filtrate was concentrated and purified by silica gel column chromatography (0-100% EtOAc:Heptane) to provide benzyl (((3S,4R)-3-fluoro-1-(6-(1-methyl-1H-pyrazol-4-yl)pyrazolo[1,5-a]pyrazin-4-yl)piperidin-4-yl)methyl)carbamate (662.0 mg, 91% yield) as a tan solid. LCMS m/z=464.2 [M+H]⁺

### 2. Preparation of ((3S,4R)-3-fluoro-1-(6-(1-methyl-1H-pyrazol-4-yl)pyrazolo[1,5-a]pyrazin-4-yl)piperidin-4-yl)methanamine

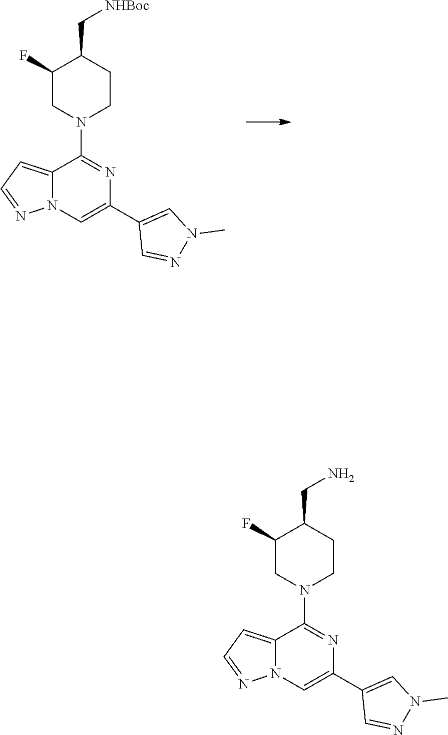 [SMILES: Cn1cc(-c2cn3nccc3c(N3CC[C@H](CNC(=O)OC(C)(C)C)[C@H](F)C3)n2)cn1 ; Cn1cc(-c2cn3nccc3c(N3CC[C@H](CN)[C@H](F)C3)n2)cn1]

A vial was charged with benzyl (((3S,4R)-3-fluoro-1-(6-(1-methyl-1H-pyrazol-4-yl)pyrazolo[1,5-a]pyrazin-4-yl)piperidin-4-yl)methyl)carbamate (653.6 mg, 1.41 mmol), palladium (300.1 mg, 141 μmol) and MeOH (7.05 mL), purged and backfilled with H₂ three times, then stirred under an atmosphere of H₂ for 4 h. The reaction was filtered through a pad of Celite®, washed with MeOH and concentrated to afford ((3S,4R)-3-fluoro-1-(6-(1-methyl-1H-pyrazol-4-yl)pyrazolo[1,5-a]pyrazin-4-yl)piperidin-4-yl)methanamine (441 mg, 95% yield) as a light yellow solid. LCMS m/z=330.2 [M+H]⁺

### 3. Preparation of 1-(tert-butyl)-N-(((3S,4R)-3-fluoro-1-(6-(1-methyl-1H-pyrazol-4-yl)pyrazolo[1,5-a]pyrazin-4-yl)piperidin-4-yl)methyl)-1H-1,2,3-triazole-4-carboxamide

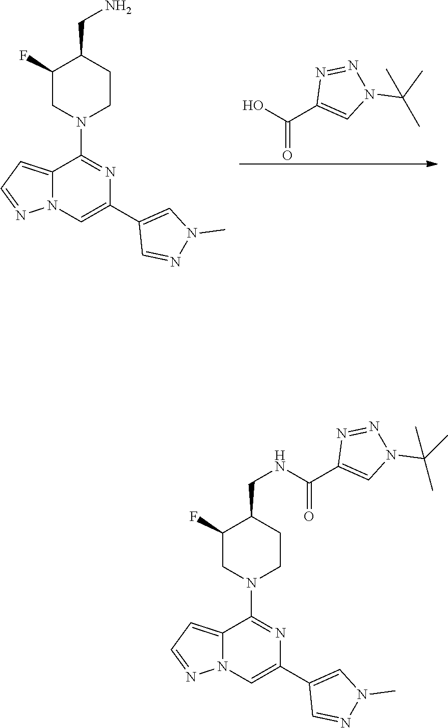 [SMILES: Cn1cc(-c2cn3nccc3c(N3CC[C@H](CN)[C@H](F)C3)n2)cn1 ; CC(C)(C)n1cc(C(=O)O)nn1 ; Cn1cc(-c2cn3nccc3c(N3CC[C@H](CNC(=O)c4cn(C(C)(C)C)nn4)[C@H](F)C3)n2)cn1]

DIPEA (147.14 mg, 1.14 mmol) was added to a solution of ((3S,4R)-3-fluoro-1-(6-(1-methyl-1H-pyrazol-4-yl)pyrazolo[1,5-a]pyrazin-4-yl)piperidin-4-yl)methanamine (75 mg, 227.7 μmol) and 1-(tert-butyl)-1H-1,2,3-triazole-4-carboxylic acid (57.79 mg, 341.6 μmol) in DMF (2.28 mL). T3P® (434.71 mg, 683.1 μmol, 50% purity) was added and the reaction stirred overnight at rt. The reaction was diluted with water and DCM and passed through a phase separator. The aqueous layer was extracted with DCM and the combined organic layers were concentrated. The residue was purified by HPLC (Method A2, 5-70%) The product containing fractions were concentrated, dissolved in MeCN (1 mL) and water (1 mL), and the resulting material was frozen in a dry ice/acetone bath and lyophilized to afford 1-(tert-butyl)-N-(((3S,4R)-3-fluoro-1-(6-(1-methyl-1H-pyrazol-4-yl)pyrazolo[1,5-a]pyrazin-4-yl)piperidin-4-yl)methyl)-1H-1,2,3-triazole-4-carboxamide (56 mg, 50.7% yield) as a white solid. LCMS m/z=481.3 [M+H]⁺ 1H NMR (400 MHz, DMSO-d₆) δ ppm 8.67 (s, 1H), 8.61 (t, J=5.9 Hz, 1H), 8.46 (d, J=0.8 Hz, 1H), 8.15 (s, 1H), 7.97-7.92 (m, 2H), 6.92 (dd, J=2.5, 0.8 Hz, 1H), 4.96 (br s, 1H), 4.88-4.76 (m, 2H), 4.67 (br d, J=12.0 Hz, 1H), 3.44-3.35 (m, 1H), 3.30-3.23 (m, 1H), 3.11-2.98 (m, 1H), 2.29-2.10 (m, 1H), 1.76-1.67 (m, 2H), 1.64 (s, 9H).

### Example 153: 2-(tert-butyl)-N-(((3S,4R)-3-fluoro-1-(6-(1-methyl-1H-pyrazol-4-yl)pyrazolo[1,5-a]pyrazin-4-yl)piperidin-4-yl)methyl)oxazole-4-carboxamide

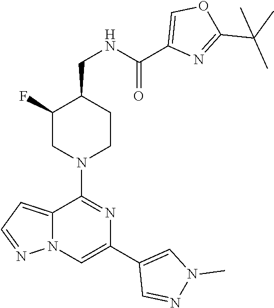 [SMILES: Cn1cc(-c2cn3nccc3c(N3CC[C@H](CNC(=O)c4coc(C(C)(C)C)n4)[C@H](F)C3)n2)cn1]

2-(tert-Butyl)-N-(((3S,4R)-3-fluoro-1-(6-(1-methyl-1H-pyrazol-4-yl)pyrazolo[1,5-a]pyrazin-4-yl)piperidin-4-yl)methyl)oxazole-4-carboxamide was obtained as a white solid, 44 mg, 39.8% yield, from ((3S,4R)-3-fluoro-1-(6-(1-methyl-1H-pyrazol-4-yl)pyrazolo[1,5-a]pyrazin-4-yl)piperidin-4-yl)methanamine (Example 152, step 2) and 2-tert-butyloxazole-4-carboxylic acid, following the procedure described in Example 152, step 3. LCMS m/z=481.3 [M+H]⁺. ¹H NMR (400 MHz, DMSO-d₆) δ ppm 8.49 (s, 1H), 8.46 (d, J=0.8 Hz, 1H), 8.20 (br t, J=6.0 Hz, 1H), 8.15 (s, 1H), 7.99-7.91 (m, 2H), 6.92 (d, J=2.5 Hz, 1H), 4.94 (br s, 1H), 4.88-4.76 (m, 2H), 4.66 (br d, J=11.8 Hz, 1H), 3.87 (s, 3H), 3.44-3.33 (m, 2H), 3.30-3.20 (m, 2H), 3.04 (br s, 1H), 2.27-2.08 (m, 2H), 1.69 (br s, 2H), 1.36 (s, 9H).

### Example 154: N-(((3S,4R)-3-fluoro-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide

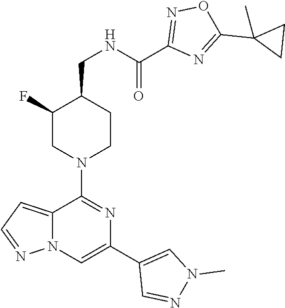 [SMILES: Cn1cc(-c2cn3nccc3c(N3CC[C@H](CNC(=O)c4noc(C5(C)CC5)n4)[C@H](F)C3)n2)cn1]

N-(((3S,4R)-3-Fluoro-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide was obtained as a white solid, 29 mg, 26.3% yield, from ((3S,4R)-3-fluoro-1-(6-(1-methyl-1H-pyrazol-4-yl)pyrazolo[1,5-a]pyrazin-4-yl)piperidin-4-yl)methanamine (Example 152, step 2) and 5-(1-methylcyclopropyl)-1,2,4-oxadiazole-3-carboxylic acid (Example 137, step 3), following the procedure described in Example 152, step 3. LCMS m/z=480.2 [M+H]⁺ 1H NMR (400 MHz. DMSO-d₆) δ ppm 8.96-9.04 (m, 1H), 8.47 (s, 1H), 8.15 (s. 1H), 797-7.92 (m, 2H), 6.92 (d, J=2.5 Hz, 1H), 4.94 (br s, 1H), 4.87-4.76 (n, 2H), 4,67 (br d, J=15.1 Hz, 1 [H), 3.87 (s, 3H), 3.42-3.34 (m, 1H), 3.26 (s, 1H), 3.05 (s, 1H), 2.27-2.08 (m. 2H), 1.70 (br s, 2H), 154 (s, 3H), 1.42-1.34 (m, 2H), 1.21-1.11 (nm, 2H).

### Example 155 and 156: 5-(tert-butyl)-N-(((3S,4R)-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide and 5-(tert-butyl)-N-(((3R,4S)-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

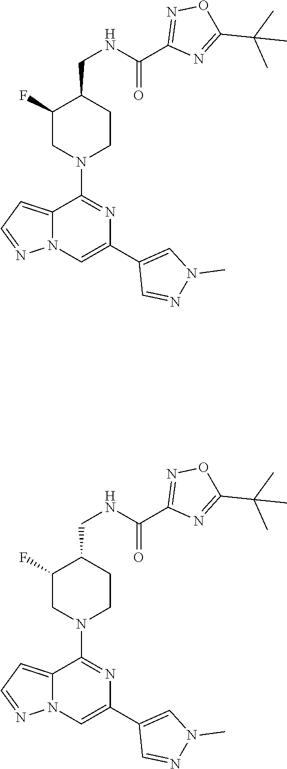 [SMILES: Cn1cc(-c2cn3nccc3c(N3CC[C@H](CNC(=O)c4noc(C(C)(C)C)n4)[C@H](F)C3)n2)cn1 ; Cn1cc(-c2cn3nccc3c(N3CC[C@@H](CNC(=O)c4noc(C(C)(C)C)n4)[C@@H](F)C3)n2)cn1]

### 1. Preparation of tert-butyl cis-4-((5-(tert-butyl)-1,2,4-oxadiazole-3-carboxamido)methyl)-3-fluoropiperidine-1-carboxylate

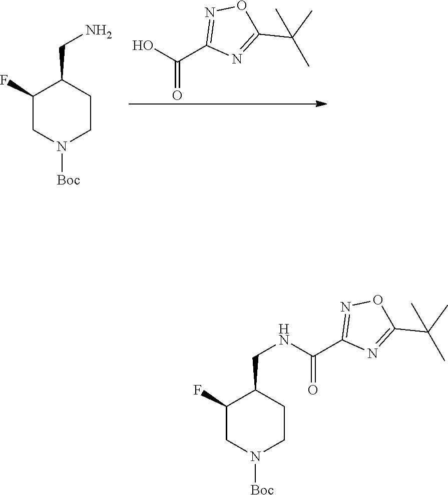 [SMILES: CC(C)(C)OC(=O)N1CC[C@H](CN)[C@H](F)C1 ; CC(C)(C)c1nc(C(=O)O)no1 ; CC(C)(C)OC(=O)N1CC[C@H](CNC(=O)c2noc(C(C)(C)C)n2)[C@H](F)C1]

DIPEA (833.6 mg, 6.45 mmol) was added to a solution of tert-butyl cis-4-(aminomethyl)-3-fluoro-piperidine-1-carboxylate (499.42 mg, 2.15 mmol) and potassium 5-tert-butyl-1,2,4-oxadiazole-3-carboxylate (539.89 mg, 2.58 mmol) in DCM (21.5 mL) and the solution cooled to 0° C. HATU (983.60 mg, 2.58 mmol) was added in a single portion and the reaction was stirred overnight at rt. The reaction was concentrated and purified by column chromatography (40 g column, gradient elution 0-100% EtOAc:Heptane) to afford tert-butyl cis-4-((5-(tert-butyl)-1,2,4-oxadiazole-3-carboxamido)methyl)-3-fluoropiperidine-1-carboxylate (405 mg, 49% yield) as a white solid. LCMS m/z=407.2 [M+H]⁺

### 2. Preparation of 5-(tert-butyl)-N-((cis-3-fluoropiperidin-4-yl)methyl)-1,2,4-oxadiazole-3-carboxamide hydrochloride

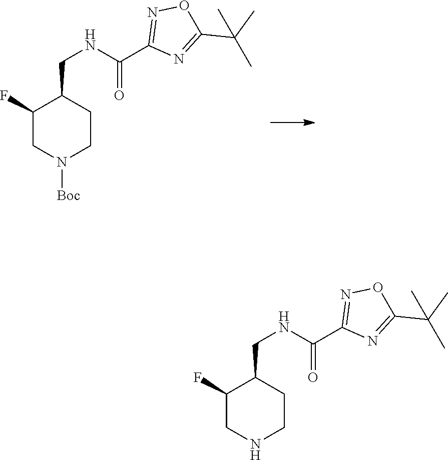 [SMILES: CC(C)(C)OC(=O)N1CC[C@H](CNC(=O)c2noc(C(C)(C)C)n2)[C@H](F)C1 ; CC(C)(C)c1nc(C(=O)NC[C@H]2CCNC[C@H]2F)no1]

5-(tert-Butyl)-N-((cis-3-fluoropiperidin-4-yl)methyl)-1,2,4-oxadiazole-3-carboxamide hydrochloride (403.7 mg, 1.05 mmol) was dissolved in MeOH (10.5 mL) and 1.25M HCl solution (in MeOH, 8.40 mL) and the reaction was stirred overnight at 50° C. The reaction was concentrated and the residue vacuum dried to afford 5-(tert-butyl)-N-((cis-3-fluoropiperidin-4-yl)methyl)-1,2,4-oxadiazole-3-carboxamide hydrochloride (346 mg, crude) as a white solid. LCMS m/z=285.1 [M+H]⁺

### 3. Preparation of 5-(tert-butyl)-N-((cis-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

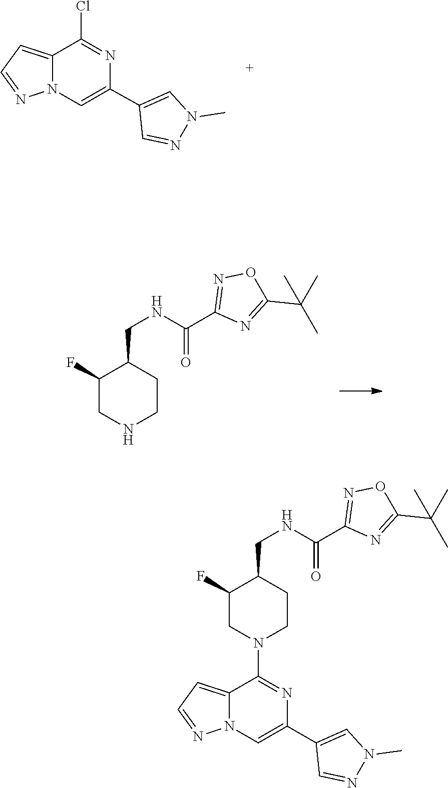 [SMILES: Cn1cc(-c2cn3nccc3c(Cl)n2)cn1 ; CC(C)(C)c1nc(C(=O)NC[C@H]2CCNC[C@H]2F)no1 ; Cn1cc(-c2cn3nccc3c(N3CC[C@H](CNC(=O)c4noc(C(C)(C)C)n4)[C@H](F)C3)n2)cn1]

A mixture of 5-(tert-butyl)-N-((cis-3-fluoropiperidin-4-yl)methyl)-1,2,4-oxadiazole-3-carboxamide hydrochloride (50 mg, 155.9 μmol), 4-chloro-6-(1-methylpyrazol-4-yl)pyrazolo[1,5-a]pyrazine (Example 1, step 5, 36.42 mg, 155.9 μmol) and DIPEA (80.58 mg, 623.5 μmol) were dissolved in DMF (311.7 uL) and the reaction was stirred at 80° C. overnight. The reaction was concentrated and the residue purified by HPLC (Method A2, 5-60%) afford 5-(tert-butyl)-N-((cis-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (36 mg, 48.0% yield) as a beige solid. LCMS m/z=482.2 [M+H]⁺4. Separation of 5-(tert-butyl)-N-((cis-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

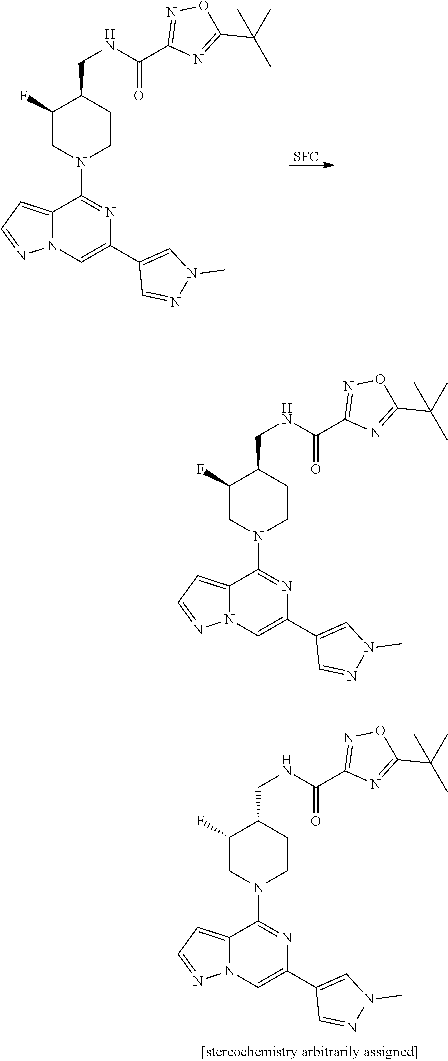 [SMILES: Cn1cc(-c2cn3nccc3c(N3CC[C@H](CNC(=O)c4noc(C(C)(C)C)n4)[C@H](F)C3)n2)cn1 ; "SFC" ; Cn1cc(-c2cn3nccc3c(N3CC[C@H](CNC(=O)c4noc(C(C)(C)C)n4)[C@H](F)C3)n2)cn1 ; Cn1cc(-c2cn3nccc3c(N3CC[C@@H](CNC(=O)c4noc(C(C)(C)C)n4)[C@@H](F)C3)n2)cn1]

5-(tert-Butyl)-N-((cis-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (34 mg) was purified by SFC CHIRALPAK AD-H 30×250 mm, Sum Method: 45% IPA w/0.1% DEA in CO₂ (flow rate: 100 mL/min, ABPR 120bar, MBPR 60 psi, column temp 40° C.) to provide:

Peak 1, 5-(tert-butyl)-N-(((3S,4R)-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (5.0 mg, purity 98%). LCMS m/z=482.3 [M+H]^{+ 1}H NMR (600 MHz, DMSO-d₆) δ ppm 9.12-9.03 (m, 1H) 8.48 (s, 1H) 8.15 (s, 1H) 7.96 (d, J=3.67 Hz, 2H) 6.93 (d, J=2.20 Hz, 1H) 4.93 (br s, 1H) 4.87-4.77 (m, 2H) 4.67 (br d, J=11.74 Hz, 1H) 3.87 (s, 3H) 3.05 (s, 1H) 1.71 (br s, 2H) 1.43 (s, 9H) 1.23 (s, 3H) 0.85 (s, 1H)

And peak 2, 5-(tert-butyl)-N-(((3R,4S)-3-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (4.0 mg, 98% purity). LCMS m/z=482.3 [M+H]^{+ 1}H NMR (600 MHz, DMSO-d₆) δ ppm 9.13-9.05 (m, 1H) 8.48 (s, 1H) 8.16 (s, 1H) 7.99-7.93 (m, 2H) 6.93 (d, J=1.47 Hz, 1H) 4.93 (br s, 1H), 4.87-4.78 (m, 2H) 4.67 (br d, J=13.21 Hz, 1H) 3.87 (s, 3H) 3.04 (br d, J=14.67 Hz, 1H) 1.71 (br s, 2H) 1.43 (s, 9H) 1.23 (s, 3H).

### Example 157: 5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

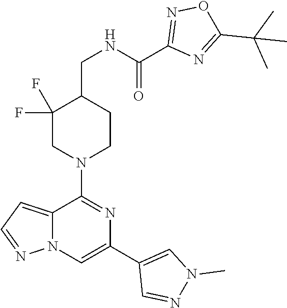 [SMILES: Cn1cc(-c2cn3nccc3c(N3CCC(CNC(=O)c4noc(C(C)(C)C)n4)C(F)(F)C3)n2)cn1]

### 1. Preparation of tert-butyl 4-((5-(tert-butyl)-1,2,4-oxadiazole-3-carboxamido)methyl)-3,3-difluoropiperidine-1-carboxylate

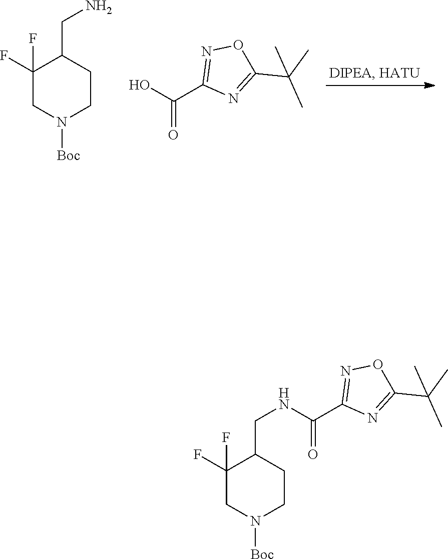 [SMILES: CC(C)(C)OC(=O)N1CCC(CN)C(F)(F)C1 ; CC(C)(C)c1nc(C(=O)O)no1 ; CC(C)(C)OC(=O)N1CCC(CNC(=O)c2noc(C(C)(C)C)n2)C(F)(F)C1]

tert-Butyl 4-((5-(tert-butyl)-1,2,4-oxadiazole-3-carboxamido)methyl)-3,3-difluoropiperidine-1-carboxylate was obtained as a white solid, 558 mg, 54.6% yield from tert-butyl 4-(aminomethyl)-3,3-difluoro-piperidine-1-carboxylate and potassium 5-tert-butyl-1,2,4-oxadiazole-3-carboxylate following the procedure described in Preparation 155 and 156, step 1. LCMS m/z=452.2 [M+Na]+

### 2. Preparation of 5-(tert-butyl)-N-((3,3-difluoropiperidin-4-yl)methyl)-1,2,4-oxadiazole-3-carboxamide hydrochloride

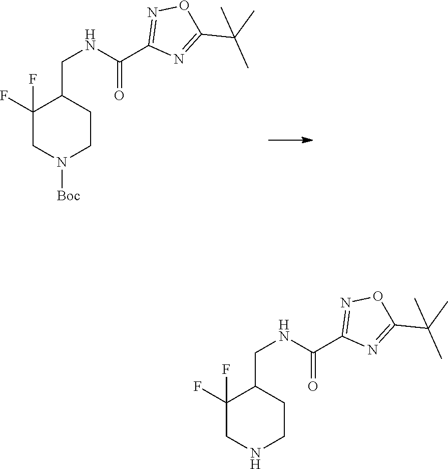 [SMILES: CC(C)(C)OC(=O)N1CCC(CNC(=O)c2noc(C(C)(C)C)n2)C(F)(F)C1 ; CC(C)(C)c1nc(C(=O)NCC2CCNCC2(F)F)no1]

tert-Butyl 4-((5-(tert-butyl)-1,2,4-oxadiazole-3-carboxamido)methyl)-3,3-difluoropiperidine-1-carboxylate (559.39 mg, 1.39 mmol) was dissolved in MeOH (13.90 mL), HCl solution (1.25M in MeOH, 11.12 mL) was added and the reaction was stirred overnight at 50° C. The reaction was concentrated and vacuum dried to afford crude 5-tert-butyl-N-[(3,3-difluoro-4-piperidyl)methyl]-1,2,4-oxadiazole-3-carboxamide hydrochloride (476 mg, crude) as a white solid. LCMS m/z=303.1 [M+H]⁺

### 3. Preparation of 5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

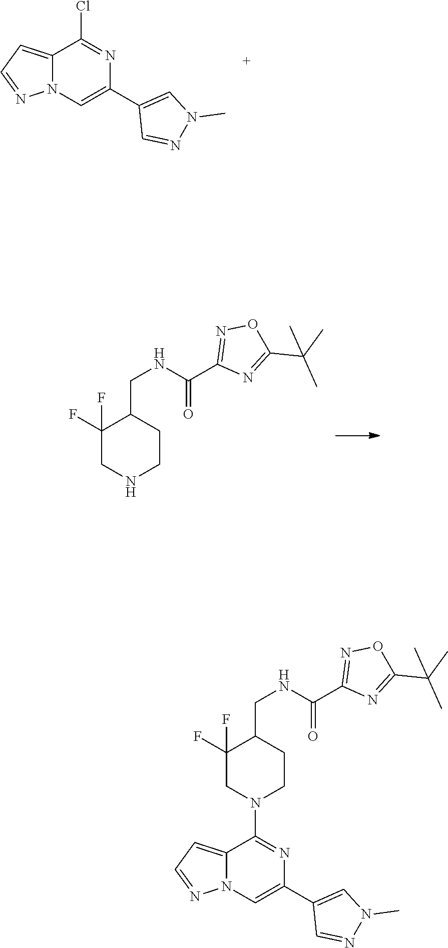 [SMILES: Cn1cc(-c2cn3nccc3c(Cl)n2)cn1 ; CC(C)(C)c1nc(C(=O)NCC2CCNCC2(F)F)no1 ; Cn1cc(-c2cn3nccc3c(N3CCC(CNC(=O)c4noc(C(C)(C)C)n4)C(F)(F)C3)n2)cn1]

A mixture of 5-(tert-butyl)-N-((3,3-difluoropiperidin-4-yl)methyl)-1,2,4-oxadiazole-3-carboxamide hydrochloride (50 mg), 4-chloro-6-(1-methylpyrazol-4-yl)pyrazolo[1,5-a]pyrazine (Example 1, step 5, 38.64 mg, 165.4 μmol) and DIPEA (85.50 mg, 661.6 μmol) were dissolved in DMF (330.78 uL) and the reaction was stirred at 80° C. overnight and a further 24 h at 100° C. The reaction was diluted with DMSO, passed through a syringe filter and purified by HPLC (Method C3, 5-60%) to afford 5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (30 mg, 36.3% yield) as a white solid. LCMS m/z=500.2 [M+H]^{+ 1}H NMR (500 MHz, DMSO-d₆) δ ppm 9.07 (br t, J=6.1 Hz, 1H), 8.56 (s, 1H), 8.19 (s, 1H), 8.00-7.96 (m, 1H), 7.00 (d, J=2.4 Hz, 1H), 4.73-4.63 (m, 2H), 4.51 (br d, J=13.4 Hz, 2H), 3.88 (s, 2H), 3.69-3.61 (m, 2H), 3.57 (br d, J=14.0 Hz, 1H), 3.48 (br s, 1H), 3.33 (ddd, J=13.4, 9.2, 6.7 Hz, 1H), 3.21 (br t, J=11.6 Hz, 2H), 1.99 (br d, J=14.0 Hz, 2H), 1.74-1.61 (m, 2H), 1.43 (s, 7H)

### Example 158 and 159: (S)-5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide and (R)-5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

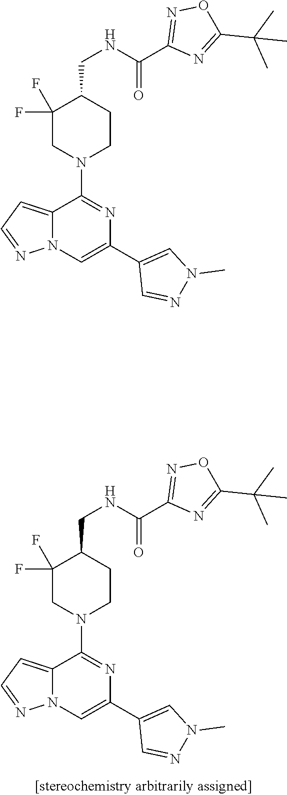 [SMILES: Cn1cc(-c2cn3nccc3c(N3CC[C@@H](CNC(=O)c4noc(C(C)(C)C)n4)C(F)(F)C3)n2)cn1 ; Cn1cc(-c2cn3nccc3c(N3CC[C@H](CNC(=O)c4noc(C(C)(C)C)n4)C(F)(F)C3)n2)cn1]

5-(tert-Butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (Example 157, 29 mg) was purified by chiral SFC (LUX Cellulose-4 LC 30×250 mm, 3 um), Method: 45% MeOH w/0.1% DEA in C₀₂ (flow rate: 100 mL/min, ABPR 120bar, MBPR 40 psi, column temp 40° C.) to provide:

Peak 1, (S)-5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (10 mg, 34.5% yield) LCMS m/z=500.2 [M+H]⁺. 1H NMR (500 MHz, DMSO-d₆) δ ppm 9.11-9.05 (m, 1H), 8.56 (s, 1H), 8.19 (s, 1H), 7.99 (d, J=2.4 Hz, 1H), 7.98 (s, 1H), 7.00 (br d, J=2.4 Hz, 1H), 4.66 (br d, J=14.7 Hz, 2H), 4.51 (br d, J=13.4 Hz, 2H), 3.88 (s, 3H), 3.70-3.61 (m, 2H), 3.26-3.19 (m, 1H), 2.01-1.97 (m, 1H), 1.74-1.66 (m, 1H), 1.43 (s, 9H)

And Peak 2, (R)-5-(tert-butyl)-N-((3,3-difluoro-1-(6-(1-methyl-1H-pyrazol-4-yl)pyrazolo[1,5-a]pyrazin-4-yl)piperidin-4-yl)methyl)-1,2,4-oxadiazole-3-carboxamide (10 mg, 34.5% yield). LCMS m/z=500.2 [M+H]^{+ 1}H NMR (500 MHz, DMSO-d₆) δ ppm 9.11-9.04 (m, 1H), 8.56 (s, 1H), 8.19 (s, 1H), 7.99 (d, J=2.4 Hz, 1H), 7.98 (s, 1H), 7.00 (br d, J=2.4 Hz, 1H), 4.67 (br s, 2H), 4.51 (br d, J=13.4 Hz, 2H), 3.69-3.60 (m, 2H), 3.57 (br d, J=14.0 Hz, 1H), 3.24-3.17 (m, 1H), 2.03-1.95 (m, 1H), 1.73-1.65 (m, 1H), 1.43 (s, 9H)

### Example 160: 5-(tert-butyl)-N-((4-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide

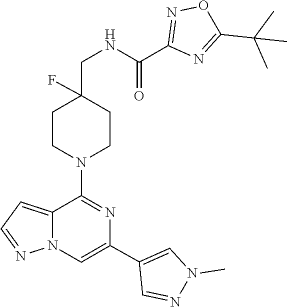 [SMILES: Cn1cc(-c2cn3nccc3c(N3CCC(F)(CNC(=O)c4noc(C(C)(C)C)n4)CC3)n2)cn1]

### 1. Preparation of tert-butyl ((4-fluoro-1-(6-(1-methyl-1H-pyrazol-4-yl)pyrazolo[1,5-a]pyrazin-4-yl)piperidin-4-yl)methyl)carbamate

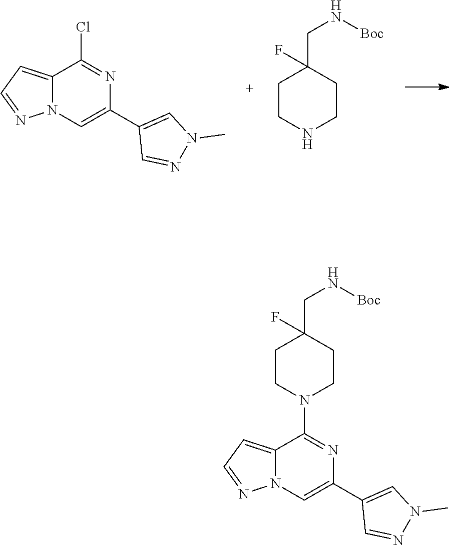 [SMILES: Cn1cc(-c2cn3nccc3c(Cl)n2)cn1 ; CC(C)(C)OC(=O)NCC1(F)CCNCC1 ; Cn1cc(-c2cn3nccc3c(N3CCC(F)(CNC(=O)OC(C)(C)C)CC3)n2)cn1]

A mixture of tert-butyl ((4-fluoropiperidin-4-yl)methyl)carbamate (238.59 mg, 1.03 mmol), 4-chloro-6-(1-methylpyrazol-4-yl)pyrazolo[1,5-a]pyrazine (Example 1, step 5, 200 mg, 855.9 μmol) and DIPEA (442.49 mg, 3.42 mmol) were dissolved in DMF (1.71 mL) and the reaction was stirred at 100° C. overnight. The reaction was diluted with water and extracted twice with EtOAc. The combined organic layers were washed with brine, dried over Na₂SO₄, filtered and concentrated. The material was purified by column chromatography (0-75% [3:1 EtOAc:EtOH]:Heptane) to afford tert-butyl ((4-fluoro-1-(6-(1-methyl-1H-pyrazol-4-yl)pyrazolo[1,5-a]pyrazin-4-yl)piperidin-4-yl)methyl)carbamate (439 mg, impure) as an off-white solid. LCMS m/z=430.3 [M+H]⁺

### 2. Preparation of (4-fluoro-1-(6-(1-methyl-1H-pyrazol-4-yl)pyrazolo[1,5-a]pyrazin-4-yl)piperidin-4-yl)methanamine dihydrochloride

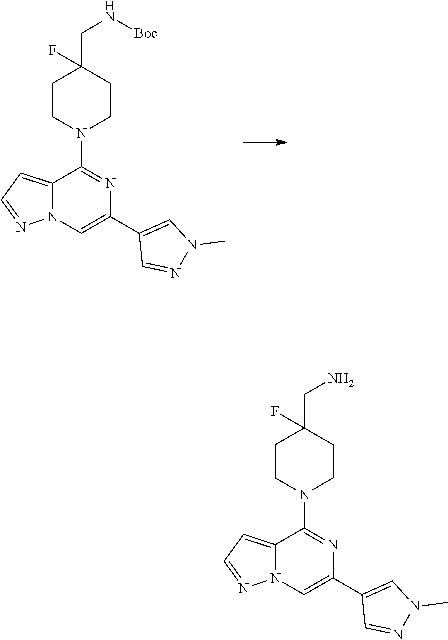 [SMILES: Cn1cc(-c2cn3nccc3c(N3CCC(F)(CNC(=O)OC(C)(C)C)CC3)n2)cn1 ; Cn1cc(-c2cn3nccc3c(N3CCC(F)(CN)CC3)n2)cn1]

(4-Fluoro-1-(6-(1-methyl-1H-pyrazol-4-yl)pyrazolo[1,5-a]pyrazin-4-yl)piperidin-4-yl)methanamine dihydrochloride was obtained as a yellow solid, from tert-butyl ((4-fluoro-1-(6-(1-methyl-1H-pyrazol-4-yl)pyrazolo[1,5-a]pyrazin-4-yl)piperidin-4-yl)methyl)carbamate, following the procedure described in Example 119, step 2. LCMS m/z=330.3 [M+H]⁺

### 3. Preparation of 5-(tert-butyl)-N-((4-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide trifluoroacetate

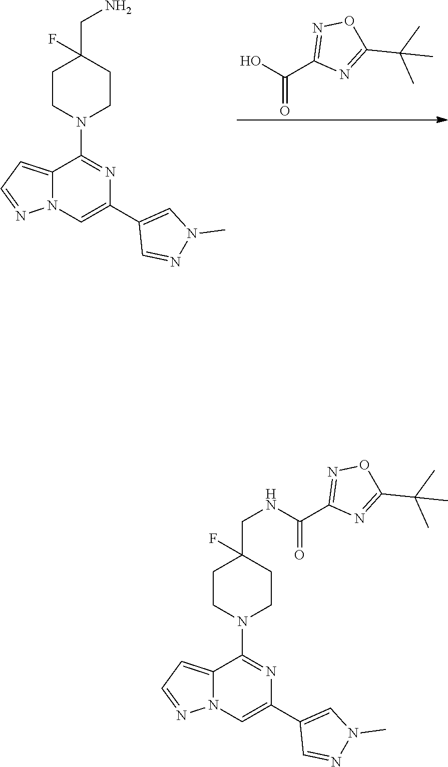 [SMILES: Cn1cc(-c2cn3nccc3c(N3CCC(F)(CN)CC3)n2)cn1 ; CC(C)(C)c1nc(C(=O)O)no1 ; Cn1cc(-c2cn3nccc3c(N3CCC(F)(CNC(=O)c4noc(C(C)(C)C)n4)CC3)n2)cn1]

DIPEA (128.50 mg, 994.3 μmol) was added to a solution of (4-fluoro-1-(6-(1-methyl-1H-pyrazol-4-yl)pyrazolo[1,5-a]pyrazin-4-yl)piperidin-4-yl)methanamine dihydrochloride (100 mg, 198.9 μmol) and potassium 5-tert-butyl-1,2,4-oxadiazole-3-carboxylate (62.42 mg, 298.3 μmol) in DMF (1.99 mL) T3P® (596.6 μmol, 355.13 uL, 50% purity) was added and the reaction was stirred overnight at rt. The reaction was diluted with water and DCM and passed through a phase separator. The aqueous layer was extracted with DCM and the combined organic layers were concentrated. The reaction was dissolved in minimal DMSO and purified by reverse phase HPLC (gradient elution 5-70% MeCN:H₂O w/0.1% TFA modifier) and the product was dissolved in MeCN (1 mL) and water (1 mL), and the resulting material was frozen in a dry ice/acetone bath and lyophilized to afford 5-(tert-butyl)-N-((4-fluoro-1-(6-(1-methyl-1H-pyrazol-4-yl)pyrazolo[1,5-a]pyrazin-4-yl)piperidin-4-yl)methyl)-1,2,4-oxadiazole-3-carboxamide trifluoroacetate (47 mg, 38.9% yield,) as a light yellow solid. LCMS m/z=482.3 [M+H]^{+ 1}H NMR (500 MHz, DMSO-d₆) δ ppm 9.07 (t, J=6.41 Hz, 1H) 8.49 (s, 1H) 8.16 (s, 1H) 7.99-7.92 (m, 2H) 6.98 (d, J=2.44 Hz, 1H) 4.37 (br d, J=13.43 Hz, 2H) 3.87 (s, 3H) 3.62-3.51 (m, 2H) 3.45-3.35 (m, 2H) 1.96-1.79 (m, 4H) 1.42 (s, 9H).

### Example 161: N-((4-fluoro-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide trifluoroacetate

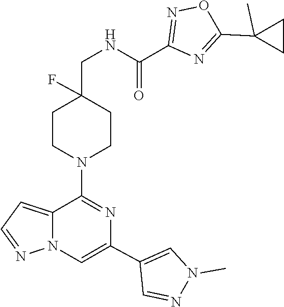 [SMILES: Cn1cc(-c2cn3nccc3c(N3CCC(F)(CNC(=O)c4noc(C5(C)CC5)n4)CC3)n2)cn1]

N-((4-Fluoro-1-(6-(1-methyl-1H-pyrazol-4-yl)pyrazolo[1,5-a]pyrazin-4-yl)piperidin-4-yl)methyl)-5-(1-methylcyclopropyl)-1,2,4-oxadiazole-3-carboxamide trifluoroacetate was obtained as a light yellow solid, 48 mg, 40.3% yield, from (4-fluoro-1-(6-(1-methyl-1H-pyrazol-4-yl)pyrazolo[1,5-a]pyrazin-4-yl)piperidin-4-yl)methanamine dihydrochloride (Example 160, step 2) and 5-(1-methylcyclopropyl)-1,2,4-oxadiazole-3-carboxylic acid (Example 137, step 3), following the procedure described in Example 160, step 3. LCMS m/z=480.2 [M+H]^{+ 1}H NMR (500 MHz, DMSO-d₆) δ ppm 9.01 (t, J=6.10 Hz, 1H) 8.49 (s, 1H) 8.16 (s, 1H) 7.99-7.93 (m, 2H) 6.97 (d, J=2.44 Hz, 1H) 4.37 (br d, J=12.82 Hz, 2H) 3.87 (s, 3H) 3.60-3.50 (m, 2H) 3.45-3.35 (m, 2H) 1.94-1.78 (m, 4H) 1.53 (s, 3H) 1.40-1.35 (m, 2H) 1.19-1.13 (m, 2H).

### Example 162: 1-(tert-butyl)-N-((4-fluoro-1-(6-(1-methyl-1H-pyrazol-4-yl)pyrazolo[1,5-a]pyrazin-4-yl)piperidin-4-yl)methyl)-1H-1,2,3-triazole-4-carboxamide trifluoroacetate

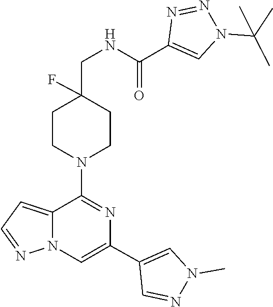 [SMILES: Cn1cc(-c2cn3nccc3c(N3CCC(F)(CNC(=O)c4cn(C(C)(C)C)nn4)CC3)n2)cn1]

1-(tert-Butyl)-N-((4-fluoro-1-(6-(1-methyl-1H-pyrazol-4-yl)pyrazolo[1,5-a]pyrazin-4-yl)piperidin-4-yl)methyl)-1H-1,2,3-triazole-4-carboxamide trifluoroacetate was obtained as a light yellow solid, 51 mg, 43.1% yield, from (4-fluoro-1-(6-(1-methyl-1H-pyrazol-4-yl)pyrazolo[1,5-a]pyrazin-4-yl)piperidin-4-yl)methanamine dihydrochloride (Example 160, step 2) and 1-tert-butyltriazole-4-carboxylic acid, following the procedure described in Example 160, step 3. LCMS m/z=481.3 [M+H]⁺. ¹H NMR (500 MHz, DMSO-d₆) δ ppm 8.71 (s, 1H) 8.52 (t, J=6.41 Hz, 1H) 8.48 (s, 1H) 8.16 (s, 1H) 7.98-7.93 (m, 2H) 6.97 (d, J=2.44 Hz, 1H) 4.37 (br d, J=12.82 Hz, 2H) 3.87 (s, 3H) 3.62-3.52 (m, 2H) 3.47-3.36 (m, 2H) 1.94-1.79 (m, 4H) 1.63 (s, 9H).

### Example 163. 1-(tert-butyl)-N-(1-(1-(6-(1-methyl-1H-pyrazol-4-yl)pyrazolo[1,5-a]pyrazin-4-yl)piperidin-4-yl)ethyl)-1H-1,2,3-triazole-4-carboxamide

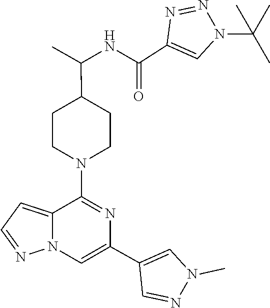 [SMILES: CC(NC(=O)c1cn(C(C)(C)C)nn1)C1CCN(c2nc(-c3cnn(C)c3)cn3nccc23)CC1]

### 1. Preparation of tert-butyl 4-(1-(1-(tert-butyl)-1H-1,2,3-triazole-4-carboxamido)ethyl)piperidine-1-carboxylate

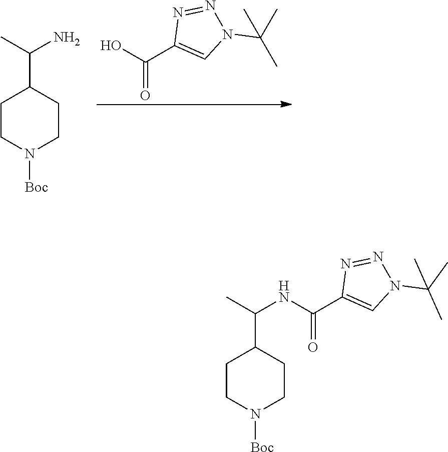 [SMILES: CC(N)C1CCN(C(=O)OC(C)(C)C)CC1 ; CC(C)(C)n1cc(C(=O)O)nn1 ; CC(NC(=O)c1cn(C(C)(C)C)nn1)C1CCN(C(=O)OC(C)(C)C)CC1]

To a solution of tert-butyl 4-(1-aminoethyl)piperidine-1-carboxylate (600 mg, 2.63 mmol) in DCM (50 mL) was added DIPEA (1.02 g, 7.89 mmol), 1-(tert-butyl)-1H-1,2,3-triazole-4-carboxylic acid (666.85 mg, 3.95 mmol) and then HATU (1.50 g, 3.95 mmol) and the reaction mixture was stirred at 20° C. for 4 h. The mixture was poured into water (200 mL) and extracted with EtOAc (100 mL×2). The combined organic layers were washed with brine (100 mL), dried over Na₂SO₄ and concentrated in vacuo. The crude product was purified by column chromatography on silica gel eluting with (PE/EtOAc=1/0˜1/1) to give tert-butyl 4-(1-(1-(tert-butyl)-1H-1,2,3-triazole-4-carboxamido)ethyl)piperidine-1-carboxylate (900 mg, 90.1% yield) as a white solid. LCMS m/z=380.2 [M+H]⁺

### 2. Preparation of 1-(tert-butyl)-N-(1-(piperidin-4-yl)ethyl)-1H-1,2,3-triazole-4-carboxamide hydrochloride

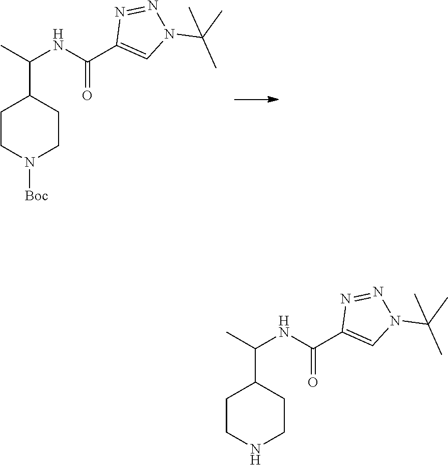 [SMILES: CC(NC(=O)c1cn(C(C)(C)C)nn1)C1CCN(C(=O)OC(C)(C)C)CC1 ; CC(NC(=O)c1cn(C(C)(C)C)nn1)C1CCNCC1]

A solution of tert-butyl 4-(1-(1-(tert-butyl)-1H-1,2,3-triazole-4-carboxamido)ethyl)piperidine-1-carboxylate (900 mg, 2.37 mmol) in HCl/EtOAc (10 mL, 4M) was stirred at 20° C. for 1 h. The mixture was concentrated in vacuo to give 1-(tert-butyl)-N-(1-(piperidin-4-yl)ethyl)-1H-1,2,3-triazole-4-carboxamide hydrochloride (800 mg, crude) as a white solid, which was used in the next step without further purification. LCMS m/z=280.1 [M+H]⁺

### 3. Preparation of 1-(tert-butyl)-N-(1-(1-(6-(1-methyl-1H-pyrazol-4-yl)pyrazolo[1,5-a]pyrazin-4-yl)piperidin-4-yl)ethyl)-1H-1,2,3-triazole-4-carboxamide

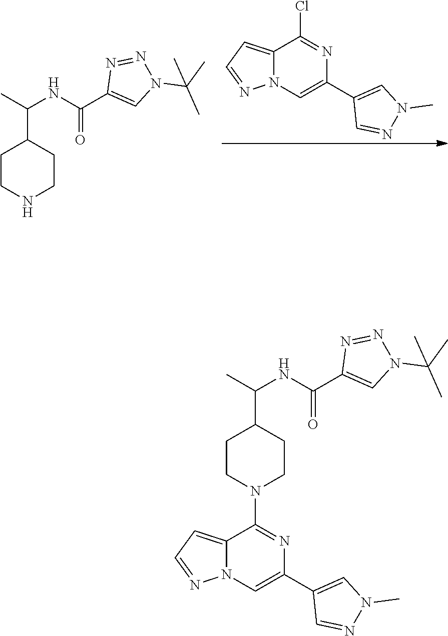

To a solution of 4-chloro-6-(1-methyl-1H-pyrazol-4-yl)pyrazolo[1,5-a]pyrazine (Example 1, step X, 150 mg, 642 μmol) and 1-(tert-butyl)-N-(1-(piperidin-4-yl)ethyl)-1H-1,2,3-triazole-4-carboxamide (358.70 mg, 1.28 mmol) in IPA (50 mL) was added DIPEA (331.87 mg, 2.57 mmol) and the reaction mixture was stirred at 90° C. for 32 h. The mixture was concentrated in vacuo to give a crude product, which was purified by prep-HPLC (Method D, 33-53% gradient) to give 1-(tert-butyl)-N-(1-(1-(6-(1-methyl-1H-pyrazol-4-yl)pyrazolo[1,5-a]pyrazin-4-yl)piperidin-4-yl)ethyl)-1H-1,2,3-triazole-4-carboxamide (108.4 mg, 35.4% yield) as a white solid. LCMS m/z=477.2 [M+H]^{+ 1}H NMR (500 MHz, DMSO-d₆) δ: 8.69 (s, 1H), 8.47 (s, 1H), 8.31 (d, J=9.0 Hz, 1H), 8.21 (s, 1H), 8.00-7.98 (m, 2H), 7.05-7.04 (m, 1H), 4.57 (t, J=11.0 Hz, 2H), 3.95-3.88 (m, 4H), 3.17-3.07 (m, 2H), 1.90-1.80 (m, 3H), 1.63 (s, 9H), 1.40-1.32 (m, 2H), 1.17 (d, J=6.5 Hz, 3H).

### Example 164 and 165: 5-(tert-butyl)-N-(((1R,5S,8s)-3-(6-(1-methyl-1H-pyrazol-4-yl)pyrazolo[1,5-a]pyrazin-4-yl)-3-azabicyclo[3.2.1]octan-8-yl)methyl)-1,2,4-oxadiazole-3-carboxamide and 5-(tert-butyl)-N-(((1R,5S,8r)-3-(6-(1-methyl-1H-pyrazol-4-yl)pyrazolo[1,5-a]pyrazin-4-yl)-3-azabicyclo[3.2.1]octan-8-yl)methyl)-1,2,4-oxadiazole-3-carboxamide

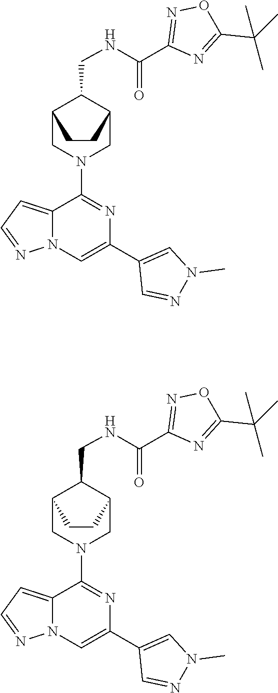 [SMILES: Cn1cc(-c2cn3nccc3c(N3C[C@H]4CC[C@@H](C3)[C@H]4CNC(=O)c3noc(C(C)(C)C)n3)n2)cn1 ; Cn1cc(-c2cn3nccc3c(N3C[C@H]4CC[C@@H](C3)[C@H]4CNC(=O)c3noc(C(C)(C)C)n3)n2)cn1]

### 1. Preparation of tert-butyl (((1R,5S,8r)-3-(6-(1-methyl-1H-pyrazol-4-yl)pyrazolo[1,5-a]pyrazin-4-yl)-3-azabicyclo[3.2.1]octan-8-yl)methyl)carbamate

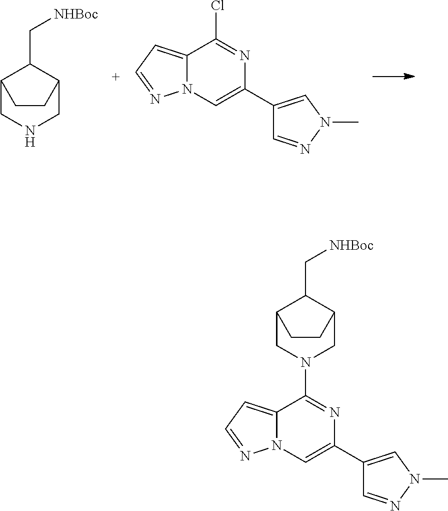 [SMILES: CC(C)(C)OC(=O)NCC1C2CCC1CNC2 ; Cn1cc(-c2cn3nccc3c(Cl)n2)cn1 ; Cn1cc(-c2cn3nccc3c(N3CC4CCC(C3)C4CNC(=O)OC(C)(C)C)n2)cn1]

tert-Butyl (((1R,5S,8r)-3-(6-(1-methyl-1H-pyrazol-4-yl)pyrazolo[1,5-a]pyrazin-4-yl)-3-azabicyclo[3.2.1]octan-8-yl)methyl)carbamate was obtained as a white solid, 445 mg, 95% yield from 4-chloro-6-(1-methylpyrazol-4-yl)pyrazolo[1,5-a]pyrazine (Example 1, step 5) and tert-butyl N-(3-azabicyclo[3.2.1]octan-8-ylmethyl)carbamate hydrochloride, following the procedure described in Example 160, step 1. LCMS m/z=438.3 [M+H]⁺

### 2. Preparation of ((1R,5S,8r)-3-(6-(1-methyl-1H-pyrazol-4-yl)pyrazolo[1,5-a]pyrazin-4-yl)-3-azabicyclo[3.2.1]octan-8-yl)methanamine hydrochloride

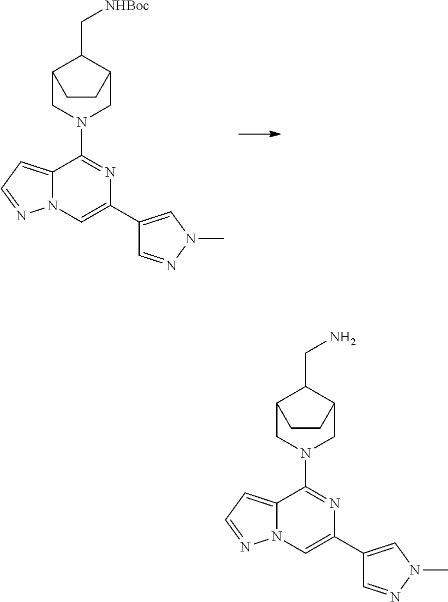 [SMILES: Cn1cc(-c2cn3nccc3c(N3CC4CCC(C3)C4CNC(=O)OC(C)(C)C)n2)cn1 ; Cn1cc(-c2cn3nccc3c(N3CC4CCC(C3)C4CN)n2)cn1]

((1R,5S,8r)-3-(6-(1-Methyl-1H-pyrazol-4-yl)pyrazolo[1,5-a]pyrazin-4-yl)-3-azabicyclo[3.2.1]octan-8-yl)methanamine hydrochloride was obtained as a light yellow solid, from tert-butyl (((1R,5S,8r)-3-(6-(1-methyl-1H-pyrazol-4-yl)pyrazolo[1,5-a]pyrazin-4-yl)-3-azabicyclo[3.2.1]octan-8-yl)methyl)carbamate, following the procedure described in Example 119, step 2. LCMS m/z=338.2 [M+H]⁺

### 3. Preparation of 5-(tert-butyl)-N—(((1R,5S,8s)-3-(6-(1-methyl-1H-pyrazol-4-yl)pyrazolo[1,5-a]pyrazin-4-yl)-3-azabicyclo[3.2.1]octan-8-yl)methyl)-1,2,4-oxadiazole-3-carboxamide and 5-(tert-butyl)-N—(((1R,5S,8r)-3-(6-(1-methyl-1H-pyrazol-4-yl)pyrazolo[1,5-a]pyrazin-4-yl)-3-azabicyclo[3.2.1]octan-8-yl)methyl)-1,2,4-oxadiazole-3-carboxamide

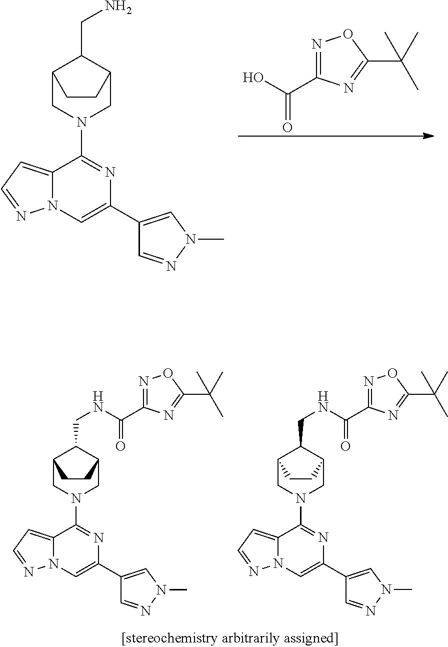 [SMILES: Cn1cc(-c2cn3nccc3c(N3CC4CCC(C3)C4CN)n2)cn1 ; CC(C)(C)c1nc(C(=O)O)no1 ; Cn1cc(-c2cn3nccc3c(N3C[C@H]4CC[C@@H](C3)[C@H]4CNC(=O)c3noc(C(C)(C)C)n3)n2)cn1 ; Cn1cc(-c2cn3nccc3c(N3C[C@H]4CC[C@@H](C3)[C@H]4CNC(=O)c3noc(C(C)(C)C)n3)n2)cn1]

DIPEA (638.4 μL, 3.66 mmol) was added to a mixture of ((1R,5S,8r)-3-(6-(1-methyl-1H-pyrazol-4-yl)pyrazolo[1,5-a]pyrazin-4-yl)-3-azabicyclo[3.2.1]octan-8-yl)methanamine hydrochloride (300 mg, 731.1 μmol) and potassium 5-tert-butyl-1,2,4-oxadiazole-3-carboxylate (229.49 mg, 1.10 mmol) in DMF (3.66 mL). T3P® (2.19 mmol, 1.31 mL, 50% purity) was added and the reaction was stirred overnight at rt. The reaction was diluted with water and DCM and passed through a phase separator. The aqueous layer was extracted with DCM and the combined organic layers were concentrated. The crude was purified by column chromatography (gradient elution 0-75% [3:1 EtOAc:EtOH]:Heptane) to afford racemic material. This was further purified by SFC (LUX Cellulose-4 LC 30×250 mm, Sum Method: 45% EtOH w/0.1% DEA in C₀₂ (flow rate: 100 mL/min, ABPR 120bar, MBPR 40 psi, column temp 40° C. to give Peak 1, 5-(tert-butyl)-N-(((1R,5S,8s)-3-(6-(1-methyl-1H-pyrazol-4-yl)pyrazolo[1,5-a]pyrazin-4-yl)-3-azabicyclo[3.2.1]octan-8-yl)methyl)-1,2,4-oxadiazole-3-carboxamide, 51 mg, LCMS m/z=490.0 [M+H]^{+ 1}H NMR (500 MHz, DMSO-d₆) δ ppm 9.12 (br t, J=5.80 Hz, 1H) 8.39 (s, 1H) 8.13 (s, 1H) 7.94 (s, 1H) 7.90 (d, J=2.44 Hz, 1H) 6.99 (d, J=1.83 Hz, 1H) 4.18 (br d, J=10.38 Hz, 2H) 3.86 (s, 3H) 3.69-3.59 (m, 4H) 2.84-2.75 (m, 1H) 2.28 (br s, 2H) 2.10-2.01 (m, 1H) 1.83-1.74 (m, 2H) 1.67-1.59 (m, 2H) 1.43 (s, 9H)

And Peak 2, 5-(tert-butyl)-N-(((1R,5S,8r)-3-(6-(1-methyl-1H-pyrazol-4-yl)pyrazolo[1,5-a]pyrazin-4-yl)-3-azabicyclo[3.2.1]octan-8-yl)methyl)-1,2,4-oxadiazole-3-carboxamide, 71 mg. LCMS m/z=490.0 [M+H]^{+ 1}H NMR (500 MHz, DMSO-d₆) δ ppm 9.09 (t, J=6.10 Hz, 1H) 8.40 (s, 1H) 8.13 (s, 1H) 7.93 (s, 1H) 7.90 (d, J=2.44 Hz, 1H) 6.96 (d, J=2.44 Hz, 1H) 4.42 (dd, J=12.82, 3.05 Hz, 2H) 3.86 (s, 3H) 3.21-3.13 (m, 4H) 2.25 (br s, 2H) 2.09 (t, J=7.94 Hz, 1H) 1.89-1.81 (m, 2H) 1.57-1.49 (m, 2H) 1.13 (t, J=7.02 Hz, 1H).

### Example 166: 5-(tert-butyl)-N-((1-(6-(1-methyl-1H-pyrazol-4-yl)pyrazolo[1,5-a]pyrazin-4-yl)azepan-4-yl)methyl)-1,2,4-oxadiazole-3-carboxamide

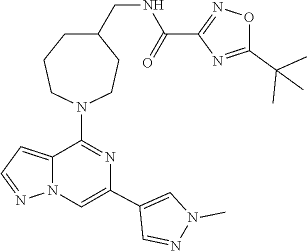 [SMILES: Cn1cc(-c2cn3nccc3c(N3CCCC(CNC(=O)c4noc(C(C)(C)C)n4)CC3)n2)cn1]

### 1. Preparation of tert-butyl ((1-(6-(1-methyl-1H-pyrazol-4-yl)pyrazolo[1,5-a]pyrazin-4-yl)azepan-4-yl)methyl)carbamate

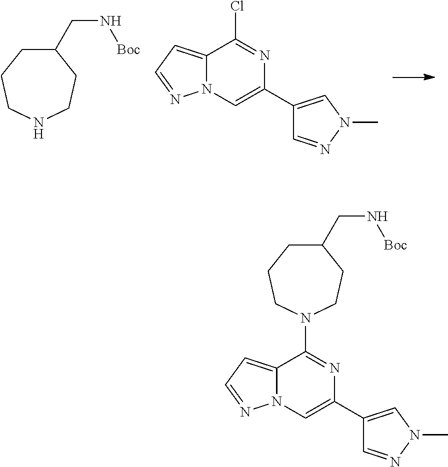

tert-Butyl ((1-(6-(1-methyl-1H-pyrazol-4-yl)pyrazolo[1,5-a]pyrazin-4-yl)azepan-4-yl)methyl)carbamate was obtained as an off-white solid, 167 mg, 91.7% yield from tert-butyl (azepan-4-ylmethyl)carbamate and 4-chloro-6-(1-methylpyrazol-4-yl)pyrazolo[1,5-a]pyrazine (Example 1, step 5), following the procedure described in Example 160, step 1. LCMS mz=426.3 [M+H]⁺

### 2. Preparation of(1-(6-(1-methyl-1H-pyrazol-4-yl)pyrazolo[1,5-a]pyrazin-4-yl)azepan-4-yl)methanamine dihydrochloride

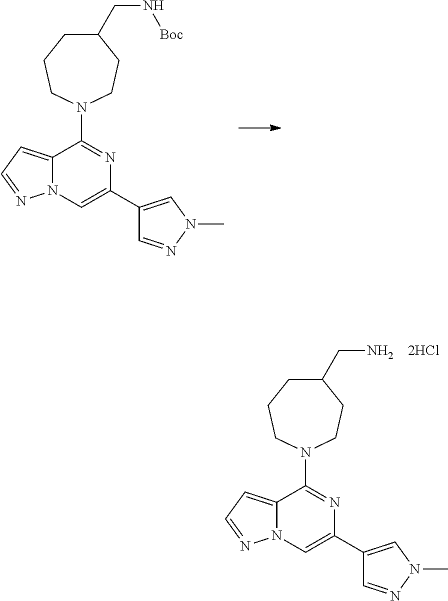 [SMILES: Cn1cc(-c2cn3nccc3c(N3CCCC(CNC(=O)OC(C)(C)C)CC3)n2)cn1 ; Cn1cc(-c2cn3nccc3c(N3CCCC(CN)CC3)n2)cn1]

(1-(6-(1-Methyl-1H-pyrazol-4-yl)pyrazolo[1,5-a]pyrazin-4-yl)azepan-4-yl)methanamine dihydrochloride was obtained as a yellow solid, 179 mg, crude, from tert-butyl ((1-(6-(1-methyl-1H-pyrazol-4-yl)pyrazolo[1,5-a]pyrazin-4-yl)azepan-4-yl)methyl)carbamate, following the procedure described in Example 23, step 3.

### 3. Preparation of 5-(tert-butyl)-N-((1-(6-(1-methyl-1H-pyrazol-4-yl)pyrazolo[1,5-a]pyrazin-4-yl)azepan-4-yl)methyl)-1,2,4-oxadiazole-3-carboxamide trifluoroacetate

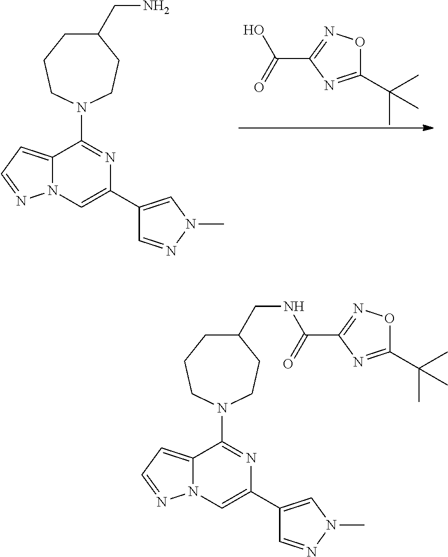 [SMILES: Cn1cc(-c2cn3nccc3c(N3CCCC(CN)CC3)n2)cn1 ; CC(C)(C)c1nc(C(=O)O)no1 ; Cn1cc(-c2cn3nccc3c(N3CCCC(CNC(=O)c4noc(C(C)(C)C)n4)CC3)n2)cn1]

5-(tert-Butyl)-N-((1-(6-(1-methyl-1H-pyrazol-4-yl)pyrazolo[1,5-a]pyrazin-4-yl)azepan-4-yl)methyl)-1,2,4-oxadiazole-3-carboxamide trifluoroacetate was obtained as a light yellow solid, 61 mg, 41.4% yield, from (1-(6-(1-methyl-1H-pyrazol-4-yl)pyrazolo[1,5-a]pyrazin-4-yl)azepan-4-yl)methanamine hydrochloride and 5-tert-butyl-1,2,4-oxadiazole-3-carboxylic acid, following the procedure described in Example 160, step 3. LCMS m/z=478.3 [M+H]⁺¹H NMR (500 MHz, DMSO-d₆) δ ppm 8.94 (t, J=5.80 Hz, 1H) 8.33 (s, 1H) 8.10 (s, 1H) 7.95-7.88 (m, 2H) 6.90 (d, J=1.83 Hz, 1H) 4.12-3.96 (m, 2H) 3.86 (s, 3H) 3.84-3.72 (m, 2H) 3.14 (t, J=6.71 Hz, 2H) 2.09-1.98 (m, 2H) 1.74 (br d, J=9.16 Hz, 3H) 1.55-1.45 (m, 1H) 1.41 (s, 9H) 1.24-1.12 (m, 1H).

### Example 167: 1-(tert-butyl)-N-((1-(6-(1-methyl-1H-pyrazol-4-yl)pyrazolo[1,5-a]pyrazin-4-yl)azepan-4-yl)methyl)-1H-1,2,3-triazole-4-carboxamide

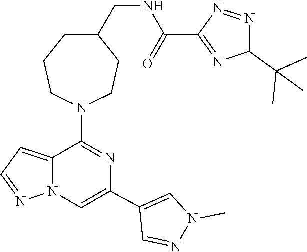 [SMILES: Cn1cc(-c2cn3nccc3c(N3CCCC(CNC(=O)C4=NC(C(C)(C)C)N=N4)CC3)n2)cn1]

1-(tert-butyl)-N-((1-(6-(1-methyl-1H-pyrazol-4-yl)pyrazolo[1,5-a]pyrazin-4-yl)azepan-4-yl)methyl)-1H-1,2,3-triazole-4-carboxamide was obtained as a yellow solid, 70 mg, 46.7% yield, from (1-(6-(1-methyl-1H-pyrazol-4-yl)pyrazolo[1,5-a]pyrazin-4-yl)azepan-4-yl)methanamine hydrochloride (Example 166, step 3) and 1-tert-butyltriazole-4-carboxylic acid, following the procedure described in Example 160, step 3. LCMS m/z=477.3 [M+H]⁺¹H NMR (500 MHz, DMSO-d₆) δ ppm 8.61 (s, 1H) 8.51 (t, J=5.80 Hz, 1H) 8.32 (s, 1H) 8.10 (s, 1H) 7.92 (s, 1H) 7.90 (d, J=2.44 Hz, 1H) 6.90 (d, J=1.83 Hz, 1H) 4.11-3.96 (m, 2H) 3.86 (s, 3H) 3.84-3.72 (m, 2H) 3.13 (t, J=6.71 Hz, 2H) 2.08-1.99 (m, 2H) 1.82-1.71 (m, 3H) 1.55-1.44 (m, 1H) 1.22-1.11 (m, 1H) 1.01-0.82 (m, 1H)

### Example 168. 4-isobutyl-1-((1-(6-(1-methyl-1H-pyrazol-4-yl)pyrazolo[1,5-a]pyrazin-4-yl)piperidin-4-yl)methyl)piperazin-2-one

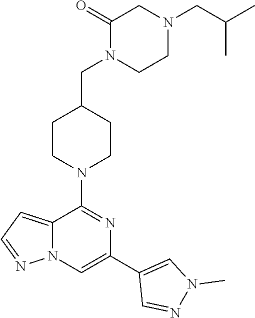 [SMILES: CC(C)CN1CCN(CC2CCN(c3nc(-c4cnn(C)c4)cn4nccc34)CC2)C(=O)C1]

### 1. Preparation of tert-butyl 4-((1-((benzyloxy)carbonyl)piperidin-4-yl)methyl)-3-oxopiperazine-1-carboxylate

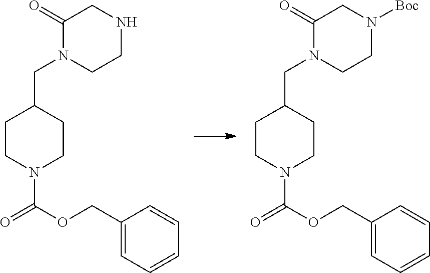 [SMILES: O=C1CNCCN1CC1CCN(C(=O)OCc2ccccc2)CC1 ; CC(C)(C)OC(=O)N1CCN(CC2CCN(C(=O)OCc3ccccc3)CC2)C(=O)C1]

To a solution of benzyl 4-((2-oxopiperazin-1-yl)methyl)piperidine-1-carboxylate (3.00 g, 9.05 mmol) in IPA (30 mL) was added DIPEA (2.34 g, 18.10 mmol) and (Boc)₂₀ (2.37 g, 10.86 mmol) and the reaction was stirred at 25° C. for 1 h. The solvent was removed under vacuum and the residue was purified by column chromatography on silica gel (from PE to PE:EtOAc=1:1) to give tert-butyl 4-((1-((benzyloxy)carbonyl)piperidin-4-yl)methyl)-3-oxopiperazine-1-carboxylate (1.20 g, 30.7% yield) as colorless oil.

### 2. Preparation of tert-butyl 3-oxo-4-(piperidin-4-ylmethyl)piperazine-1-carboxylate

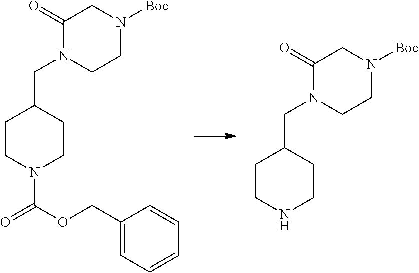 [SMILES: CC(C)(C)OC(=O)N1CCN(CC2CCN(C(=O)OCc3ccccc3)CC2)C(=O)C1 ; CC(C)(C)OC(=O)N1CCN(CC2CCNCC2)C(=O)C1]

To a solution of tert-butyl 4-((1-((benzyloxy)carbonyl)piperidin-4-yl)methyl)-3-oxopiperazine-1-carboxylate (800 mg, 1.85 mmol) in MeOH (20 mL) was added Pd/C (500 mg) and the reaction was hydrogenated at 15 psi and 30° C. for 12 h. The suspension was filtered and the filtrate was concentrated in vacuum to give tert-butyl 3-oxo-4-(piperidin-4-ylmethyl)piperazine-1-carboxylate (350 mg, crude) as a pale solid which was used for the next step without further purification.

### 3. Preparation of tert-butyl 4-((1-(6-(1-methyl-1H-pyrazol-4-yl)pyrazolo[1,5-a]pyrazin-4-yl)piperidin-4-yl)methyl)-3-oxopiperazine-1-carboxylate

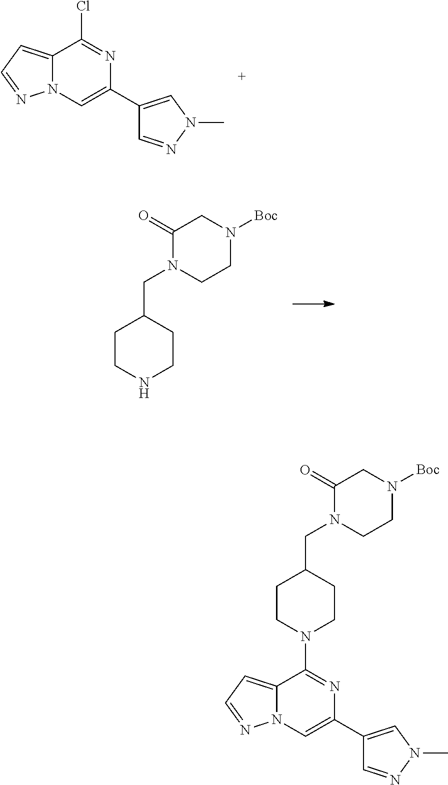 [SMILES: Cn1cc(-c2cn3nccc3c(Cl)n2)cn1 ; CC(C)(C)OC(=O)N1CCN(CC2CCNCC2)C(=O)C1 ; Cn1cc(-c2cn3nccc3c(N3CCC(CN4CCN(C(=O)OC(C)(C)C)CC4=O)CC3)n2)cn1]

To a solution of tert-butyl 3-oxo-4-(4-piperidylmethyl)piperazine-1-carboxylate (300 mg, 1.01 mmol) in butyl alcohol (10 mL) was added 4-chloro-6-(1-methylpyrazol-4-yl)pyrazolo[1,5-a]pyrazine (Example 1, step 5, 236.0 mg, 1.01 mmol) and DIPEA (352.8 μL, 2.02 mmol) and the reaction was stirred at 110° C. for 10 h. The solvent was removed under vacuum and the residue was purified by column chromatography on silica gel (PE to DCM: MeOH=10:1) to give tert-butyl 4-((1-(6-(1-methyl-1H-pyrazol-4-yl)pyrazolo[1,5-a]pyrazin-4-yl)piperidin-4-yl)methyl)-3-oxopiperazine-1-carboxylate (200 mg, 40.0% yield) as a white solid.

### 4. Preparation of 1-((1-(6-(1-methyl-1H-pyrazol-4-yl)pyrazolo[1,5-a]pyrazin-4-yl)piperidin-4-yl)methyl)piperazin-2-one hydrochloride

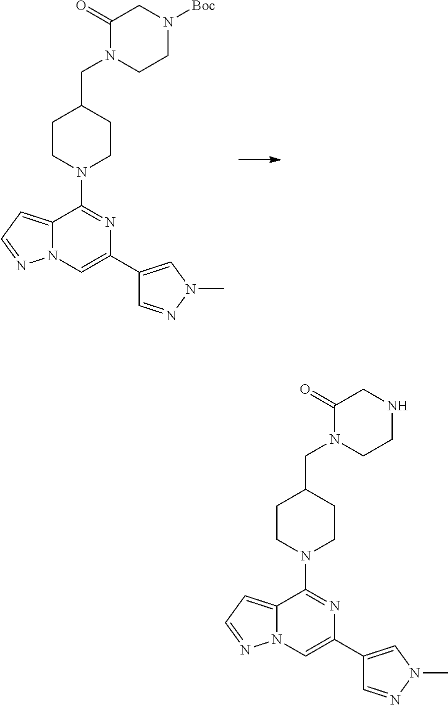 [SMILES: Cn1cc(-c2cn3nccc3c(N3CCC(CN4CCN(C(=O)OC(C)(C)C)CC4=O)CC3)n2)cn1 ; Cn1cc(-c2cn3nccc3c(N3CCC(CN4CCNCC4=O)CC3)n2)cn1]

To a solution of tert-butyl 4-((1-(6-(1-methyl-1H-pyrazol-4-yl)pyrazolo[1,5-a]pyrazin-4-yl)piperidin-4-yl)methyl)-3-oxopiperazine-1-carboxylate (80 mg, 161.8 μmol) in DCM (2.0 mL) was added HCl/EtOAc (20.0 mL) and the reaction was stirred at 25° C. for 1 h. The mixture was filtered to give 1-((1-(6-(1-methyl-1H-pyrazol-4-yl)pyrazolo[1,5-a]pyrazin-4-yl)piperidin-4-yl)methyl)piperazin-2-one hydrochloride (60 mg, crude) as a white solid. LCMS m/z=395.1 [M+H]⁺

### 5. Preparation of 4-isobutyl-1-((1-(6-(1-methyl-1H-pyrazol-4-yl)pyrazolo[1,5-a]pyrazin-4-yl)piperidin-4-yl)methyl)piperazin-2-one

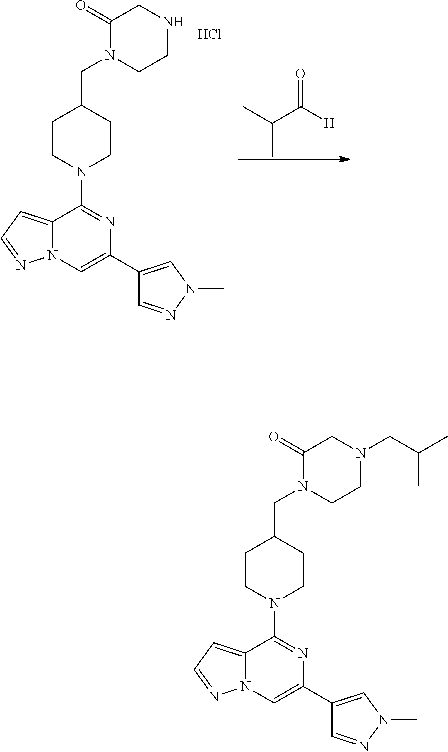

To a solution of 1-((1-(6-(1-methyl-1H-pyrazol-4-yl)pyrazolo[1,5-a]pyrazin-4-yl)piperidin-4-yl)methyl)piperazin-2-one hydrochloride (80 mg, 185.7 μmol) in MeOH (10 mL) was added DIPEA (71.98 mg, 557 μmol) at 20° C. Isobutyraldehyde (20.08 mg, 278.5 μmol) and NaBH₃CN (46.66 mg, 742.6 μmol) were added and the reaction was stirred at 20° C. for 10 h. The mixture was poured into water (50 mL) and extracted with DCM (50 mL×3). The combined organic layers were washed with brine (100 mL), dried over Na₂SO₄, filtered and concentrated in vacuo. The crude was purified by Prep-HPLC (Method D, 11-31% gradient) to give 4-isobutyl-1-((1-(6-(1-methyl-1H-pyrazol-4-yl)pyrazolo[1,5-a]pyrazin-4-yl)piperidin-4-yl)methyl)piperazin-2-one (32.7 mg, 35.2% yield) as a yellow solid. LCMS m/z=451.4 [M+H]⁺ 1H NMR (400 MHz, DMSO-d₆) δ:11.41 (s, 1H), 8.49-8.39 (m, 1H), 8.20-8.14 (m, 1H), 8.00-7.94 (m, 2H), 6.98-6.93 (m, 1H), 4.52 (s, 2H), 3.98 (s, 2H), 3.86 (s, 3H), 3.74 (s, 2H), 3.46 (s, 1H), 3.11-2.98 (s, 5H), 2.49 (s, 2H), 1.93-1.83 (m, 4H), 1.29 (s, 2H), 1.00 (s, 6H).

### Example 169: 4-(Cyclobutylmethyl)-1-((1-(6-(1-methyl-1H-pyrazol-4-yl)pyrazolo[1,5-a]pyrazin-4-yl)piperidin-4-yl)methyl)piperazin-2-one

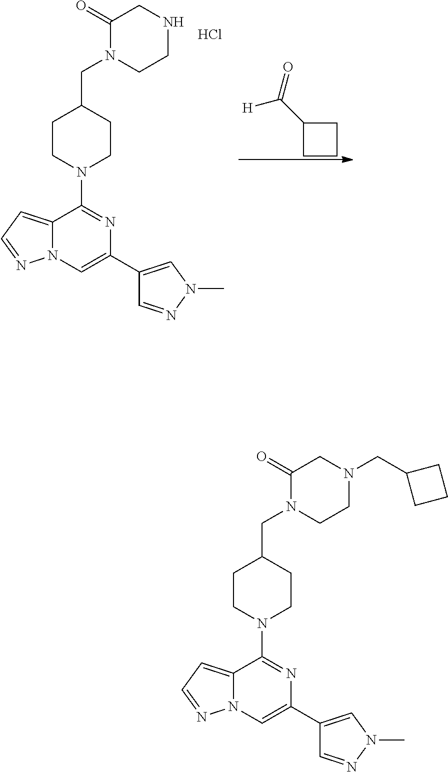 [SMILES: Cn1cc(-c2cn3nccc3c(N3CCC(CN4CCNCC4=O)CC3)n2)cn1 ; Cl ; O=CC1CCC1 ; Cn1cc(-c2cn3nccc3c(N3CCC(CN4CCN(CC5CCC5)CC4=O)CC3)n2)cn1]

4-(Cyclobutylmethyl)-1-((1-(6-(1-methyl-1H-pyrazol-4-yl)pyrazolo[1,5-a]pyrazin-4-yl)piperidin-4-yl)methyl)piperazin-2-one was obtained as a white solid, 44.8 mg, 42.5% yield, from 1-((1-(6-(1-methyl-1H-pyrazol-4-yl)pyrazolo[1,5-a]pyrazin-4-yl)piperidin-4-yl)methyl)piperazin-2-one hydrochloride (Example 168, step 4) and cyclobutanecarbaldehyde, following a similar method to that described in Example 168. The crude product was purified by Prep-HPLC (Method G, 35-65% gradient). LCMS m/z=485.2 [M+H]⁺ 1H NMR: (500 MHz, DMSO-d₆) δ=8.44 (s, 1H), 8.14 (s, 1H), 7.96-7.92 (m, 2H), 6.91 (d, J=2.0 Hz, 1H), 4.52 (d, J=13.5 Hz, 2H), 3.87 (s, 3H), 3.30-3.26 (m, 2H), 3.21 (d, J=7.5 Hz, 2H), 3.06 (t, J=12.0 Hz, 2H), 2.95 (s, 2H), 2.59 (t, J=5.5 Hz, 2H), 2.52 (d, J=1.5 Hz, 1H), 2.38 (d, J=7.0 Hz, 2H), 2.05-1.98 (m, 3H), 1.89-1.82 (m, 1H), 1.81-1.75 (m, 1H), 1.72-1.61 (m, 4H), 1.30-1.22 (m, 2H).

### Example 170: 1-((1-(6-(1-methyl-1H-pyrazol-4-yl)pyrazolo[1,5-a]pyrazin-4-yl)piperidin-4-yl)methyl)-4-propylpiperazin-2-one hydrochloride

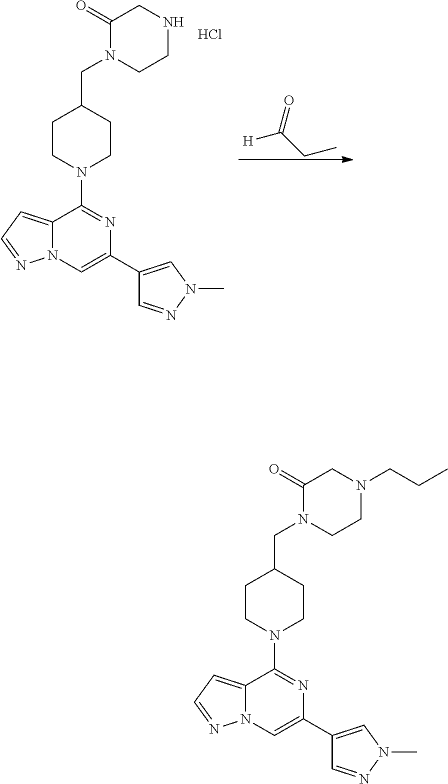

To a solution of 1-((1-(6-(1-methyl-1H-pyrazol-4-yl)pyrazolo[1,5-a]pyrazin-4-yl)piperidin-4-yl)methyl)piperazin-2-one hydrochloride (Example 168, step 4, 80 mg, 202.8 μmol) in MeOH (5.0 mL) was added propanal (47.11 mg, 811.2 μmol) and NaCNBH₃ (50.98 mg, 811.2 μmol) and the reaction was stirred at 25° C. for 10 h. The mixture was concentrated in vacuo and the crude was purified by HPLC (Method D, 10-30%) to give 1-((1-(6-(1-methyl-1H-pyrazol-4-yl)pyrazolo[1,5-a]pyrazin-4-yl)piperidin-4-yl)methyl)-4-propylpiperazin-2-one (114.3 mg, 43.8% yield) as a white solid. LCMS m/z=437.2 [M+H]⁺. 1H NMR: (500 MHz, DMSO-d₆) δ=11.5 (br s, 1H), 8.49 (s, 1H), 8.15 (s, 1H), 7.98-7.96 (m, 2H), 6.93 (s, 1H), 4.55-4.52 (m, 2H), 3.88 (s, 3H), 3.82-3.80 (m, 2H), 3.52-3.45 (m, 3H), 3.15-3.08 (m, 5H), 2.49-2.48 (m, 2H), 2.06-2.04 (m, 1H), 1.85-1.75 (m, 4H), 1.31-1.29 (m, 2H), 0.93 (t, 3H).

### Example 171: 4-methyl-1-((1-(6-(1-methyl-1H-pyrazol-4-yl)pyrazolo[1,5-a]pyrazin-4-yl)piperidin-4-yl)methyl)piperazin-2-one

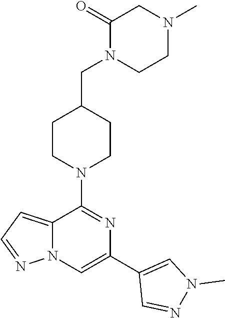 [SMILES: CN1CCN(CC2CCN(c3nc(-c4cnn(C)c4)cn4nccc34)CC2)C(=O)C1]

4-Methyl-1-((1-(6-(1-methyl-1H-pyrazol-4-yl)pyrazolo[1,5-a]pyrazin-4-yl)piperidin-4-yl)methyl)piperazin-2-one was obtained as a white solid, 43.7 mg, 52.8% yield, from 1-((1-(6-(1-methyl-1H-pyrazol-4-yl)pyrazolo[1,5-a]pyrazin-4-yl)piperidin-4-yl)methyl)piperazin-2-one hydrochloride (Example 168, step 4) and formaldehyde, following a similar procedure to that described in Example 170. The crude product was purified by HPLC (Method D, 23-32% gradient). LCMS m/z=409.3 [M+H]^{+ 1}H NMR (500 MHz, DMSO-d₆) δ=11.70 (s, 1H), 8.45 (s, 1H), 8.14 (s, 1H), 7.96-7.94 (m, 2H), 6.92 (d, J=2.0 Hz, 1H), 4.52 (d, J=13.5 Hz, 2H), 3.87 (s, 3H), 3.80 (s, 3H), 3.50 (d, J=12.0 Hz, 2H), 3.40 (d, J=7.5 Hz, 2H), 3.20 (d, J=6.0 Hz, 1H), 3.08 (d, J=7.0 Hz, 2H), 2.82 (s, 3H), 2.06-2.03 (m, 1H), 1.86-1.77 (m, 2H), 1.32-1.23 (m, 2H).

### Example 172: 1-((1-(6-(1-methyl-1H-pyrazol-4-yl)pyrazolo[1,5-a]pyrazin-4-yl)piperidin-4-yl)methyl)-4-neopentylpiperazin-2-one

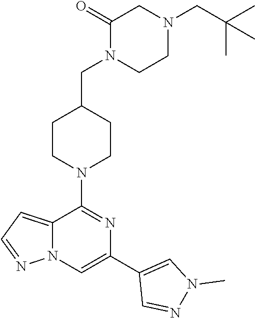 [SMILES: Cn1cc(-c2cn3nccc3c(N3CCC(CN4CCN(CC(C)(C)C)CC4=O)CC3)n2)cn1]

1-((1-(6-(1-Methyl-1H-pyrazol-4-yl)pyrazolo[1,5-a]pyrazin-4-yl)piperidin-4-yl)methyl)-4-neopentylpiperazin-2-one was obtained as a white solid, 21.2 mg, 22.5% yield, from 1-((1-(6-(1-methyl-1H-pyrazol-4-yl)pyrazolo[1,5-a]pyrazin-4-yl)piperidin-4-yl)methyl)piperazin-2-one hydrochloride (Example 168, step 4) and 2,2-dimethylpropanal, following a similar procedure to that described in Example 170. LCMS m/z=465.2 [M+H]⁺. 1H NMR: (500 MHz, DMSO-d₆) δ=8.35 (s, 1H), 8.08 (s, 1H), 7.90-7.89 (m, 2H), 6.86 (s, 1H), 4.52-4.50 (m, 2H), 3.89 (s, 3H), 3.34-3.29 (m, 5H), 3.20-3.15 (m, 2H), 2.67-2.65 (m, 2H), 2.40-2.37 (m, 2H), 2.20-2.10 (m, 1H), 1.79-1.76 (m, 2H), 1.65-1.63 (m, 1H), 1.38 (s, 9H).

### Example 173: 4-isopropyl-1-((1-(6-(1-methyl-1H-pyrazol-4-yl)pyrazolo[1,5-a]pyrazin-4-yl)piperidin-4-yl)methyl)piperazin-2-one

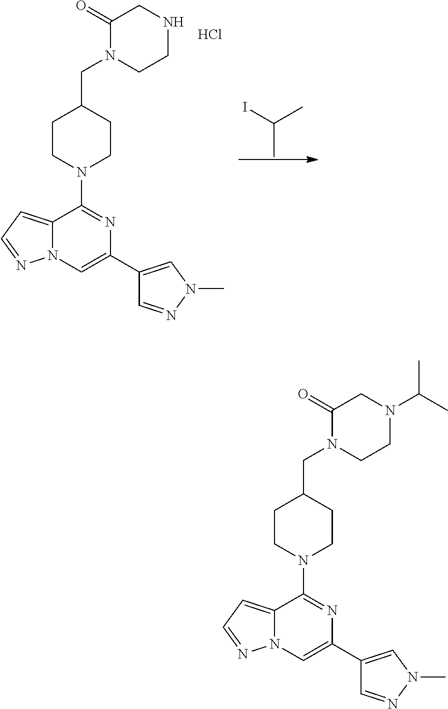

To a solution of 1-((1-(6-(1-methyl-1H-pyrazol-4-yl)pyrazolo[1,5-a]pyrazin-4-yl)piperidin-4-yl)methyl)piperazin-2-one hydrochloride (Example 168, step 4, 60 mg, crude) in MeCN (15 mL) was added K₂CO₃ (63.07 mg, 456.31 μmol) and 2-iodopropane (51.71 mg, 304.2 μmol) and the reaction was stirred at 90° C. for 10 h. The solvent was removed under vacuum and the residue was purified by prep-HPLC (Method D, 10-30% gradient) to give 4-isopropyl-1-((1-(6-(1-methyl-1H-pyrazol-4-yl)pyrazolo[1,5-a]pyrazin-4-yl)piperidin-4-yl)methyl)piperazin-2-one (15.5 mg, 23.3% yield) as a white solid. LCMS m/z=437.1 [M+H]⁺ 1H NMR: (400 MHz, DMSO-d₆) δ=8.45 (s, 1H), 8.14 (s, 1H), 7.97-7.92 (m, 2H), 6.91 (s, 1H), 4.52 (d, J=13.2 Hz, 2H), 3.87 (s, 3H), 3.86-3.62 (m, 4H), 3.55-3.43 (m, 3H), 3.30 (d, J=11.2 Hz, 1H), 3.20-3.02 (m, 3H), 2.05 (s, 1H), 1.93-1.81 (m, 2H), 1.30 (dd, J=2.4, 6.0 Hz, 8H).

### C.6 In Vitro Assays

### In Vitro BTK Kinase Assay: Btk-PolyGAT-LS Assay

The purpose of the BTK in vitro assay is to determine compound potency against BTK through the measurement of IC₅₀. Compound inhibition is measured after monitoring the amount of phosphorylation of a fluorescein-labeled polyGAT peptide (Invitrogen PV3611) in the presence of active BTK enzyme (Upstate 14-552), ATP, and inhibitor. The BTK kinase reaction was done in a black 96 well plate (costar 3694). For a typical assay, a 24 μL aliquot of a ATP/peptide master mix (final concentration; ATP 10 PM, polyGAT 100 nM) in kinase buffer (10 mM Tris-HCl pH 7.5, 10 mM MgCl2, 200 μM Na3PO4, 5 mM DTT, 0.01% Triton X-100, and 0.2 mg/ml casein) is added to each well. Next, I pL of a 4-fold, 40× compound titration in 100% DMSO solvent is added, followed by adding 15 μL of BTK enzyme mix in 1× kinase buffer (with a final concentration of 0.25 nM). The assay is incubated for 30 minutes before being stopped with 28 μL of a 50 mM EDTA solution. Aliquots (5 uL) of the kinase reaction are transferred to a low volume white 384 well plate (Coming 3674), and 5 μL of a 2× detection buffer (Invitrogen PV3574, with 4 nM Tb—PY20 antibody, Invitrogen PV3552) is added. The plate is covered and incubated for 45 minutes at room temperature. Time resolved fluorescence (TRF) on Molecular Devices M5 (332 nm excitation; 488 nm emission; 518 nm fluorescein emission) is measured. IC₅₀ values are calculated using a four parameter fit with 100% enzyme activity determined from the DMSO control and 0% activity from the EDTA control.

Table 1 shows the activity of the selected exemplary compounds of this invention in the in vitro Btk kinase assay, wherein each compound number corresponds to the example numbers in Examples 1-173. “†” represents an IC₅₀ of greater than 1 μM and equal to or less than 10 μM. “††” represents an IC₅₀ of greater than 10 nM and equal to or less than 1 μM (10 nM<IC₅₀≤1 μM). “†††” represents an IC₅₀ of greater than 1 nM and equal to or less than 10 nM (1 nM<IC₅₀≤10 nM). “††††” represents an IC₅₀ of less than 1 nM.

TABLE 1

| | In Vitro BTK |
|---|---|
| | Kinase Assay |
| Example No. | IC₅₀ Score |
| 1 | †† |
| 2 | ††† |
| 3 | †††† |
| 4 | †††† |
| 5 | †††† |
| 6 | †††† |
| 7 | †††† |
| 8 | †††† |
| 9 | †††† |
| 10 | †††† |
| 11 | ††† |
| 12 | †††† |
| 13 | †††† |
| 14 | †††† |
| 15 | †††† |
| 16 | †††† |
| 17 | †††† |
| 18 | ††† |
| 19 | †††† |
| 20 | †††† |
| 21 | †††† |
| 22 | †††† |
| 23 | † |
| 24 | †† |
| 25 | †† |
| 26 | †† |
| 27 | † |
| 28 | † |
| 29 | † |
| 30 | ††† |
| 31 | ††† |
| 32 | †† |
| 33 | ††† |
| 34 | ††† |
| 35 | ††† |
| 36 | ††† |
| 37 | †† |
| 38 | †††† |
| 39 | †††† |
| 40 | ††† |
| 41 | ††† |
| 42 | † |
| 43 | †† |
| 44 | †† |
| 45 | †† |
| 46 | †† |
| 47 | ††† |
| 48 | †† |
| 49 | † |
| 50 | † |
| 51 | † |
| 52 | † |
| 53 | †† |
| 54 | † |
| 55 | †††† |
| 56 | †† |
| 57 | ††† |
| 58 | †††† |
| 59 | ††† |
| 60 | ††† |
| 61 | †† |
| 62 | ††† |
| 63 | ††† |
| 64 | †††† |
| 65 | †††† |
| 66 | †††† |
| 67 | †††† |
| 68 | †††† |
| 69 | †††† |
| 70 | ††† |
| 71 | †††† |
| 72 | ††† |
| 73 | ††† |
| 74 | †† |
| 75 | †† |
| 76 | †† |
| 77 | †† |
| 78 | †††† |
| 79 | †††† |
| 80 | †††† |
| 81 | †††† |
| 82 | ††† |
| 83 | ††† |
| 84 | †††† |
| 85 | ††† |
| 86 | †††† |
| 87 | †††† |
| 88 | †††† |
| 89 | †††† |
| 90 | †††† |
| 91 | ††† |
| 92 | †††† |
| 93 | †††† |
| 94 | †††† |
| 95 | ††† |
| 96 | †† |
| 97 | ††† |
| 98 | ††† |
| 99 | †††† |
| 100 | †††† |
| 101 | ††† |
| 102 | †††† |
| 103 | ††† |
| 104 | †††† |
| 105 | †††† |
| 106 | †††† |
| 107 | †††† |
| 108 | †††† |
| 109 | ††† |
| 110 | ††† |
| 111 | †† |
| 112 | ††† |
| 113 | ††† |
| 114 | †††† |
| 115 | ††† |
| 116 | ††† |
| 117 | †††† |
| 118 | ††† |
| 119 | nt |
| 120 | ††† |
| 121 | †† |
| 122 | ††† |
| 123 | †† |
| 124 | †† |
| 125 | †† |
| 126 | ††† |
| 127 | †† |
| 128 | †† |
| 129 | †† |
| 130 | ††† |
| 131 | ††† |
| 132 | ††† |
| 133 | ††† |
| 134 | ††† |
| 135 | †† |
| 136 | ††† |
| 137 | ††† |
| 138 | ††† |
| 139 | ††† |
| 140 | †† |
| 141 | †† |
| 142 | †† |
| 143 | ††† |
| 144 | ††† |
| 145 | †† |
| 146 | ††† |
| 147 | †† |
| 148 | ††† |
| 149 | ††† |
| 150 | †† |
| 151 | †† |
| 152 | ††† |
| 153 | ††† |
| 154 | †† |
| 155 | ††† |
| 156 | ††† |
| 157 | †††† |
| 158 | †††† |
| 159 | †† |
| 160 | ††† |
| 161 | ††† |
| 162 | †† |
| 163 | †† |
| 164 | †† |
| 165 | †† |
| 166 | †† |
| 167 | †† |
| 168 | †† |
| 169 | †† |
| 170 | † |
| 171 | † |
| 172 | †† |
| 173 | † |

nt: not tested

In Vitro whole blood CD69 Assay

Human heparinized venous blood from health donors was aliquoted into 96-well plate and “spiked” with serial dilutions of formula I compounds in DMSO or with DMSO without drug. The final concentration of DMSO in all wells was 0.1%. The plate was incubated at 37° C. for 30 min. Drug-containing samples were stimulated with 0.1 μg/mL mouse anti-human IgD-dextran (1A62) or 20 μg/mL polyclonal rabbit F(ab′)₂ anti-human IgD. Phosphate-buffered saline (PBS) was added to the negative control unstimulated sample and the plates were incubated overnight (18 to 22 hours) at 37° C. Cells were stained with fluorochrome-conjugated anti-CD19 and anti-CD69 antibodies. Lyse/fix solution was used to remove red blood cells by hypotonic lysis and to fix the remaining cells, which were then analyzed by flow cytometry. CD19+ B cells were gated and analyzed for CD69 expression. The percentage of B cells expressing CD69 was plotted versus the log 10 of the concentration of the drug and the best-fit curves (variable Hill slope) were generated to obtain the IC₅₀ value.

Table 2 shows the activity of the selected exemplary compounds of this invention in the in vitro whole blood CD69 assay, wherein each compound number corresponds to the example numbering set forth in the Examples 1-173 herein. “t” represents an IC₅₀ of greater than 10 μM. “††” represents an IC₅₀ of greater than 1 μM and equal to or less than 10 μM (1 μM<IC₅₀≤10 μM). “†††” represents an IC₅₀ of less than 1 μM.

TABLE 2

| | In Vitro HWB |
|---|---|
| | CD69 Assay |
| Example No. | IC₅₀ Score |
| 1 | †† |
| 2 | ††† |
| 3 | ††† |
| 4 | ††† |
| 5 | ††† |
| 6 | ††† |
| 7 | ††† |
| 8 | ††† |
| 9 | ††† |
| 10 | ††† |
| 11 | †† |
| 12 | ††† |
| 13 | ††† |
| 14 | ††† |
| 15 | ††† |
| 16 | ††† |
| 17 | ††† |
| 18 | ††† |
| 19 | ††† |
| 20 | ††† |
| 21 | †† |
| 22 | ††† |
| 23 | †† |
| 24 | †† |
| 25 | †† |
| 26 | nt |
| 27 | nt |
| 28 | nt |
| 29 | nt |
| 30 | †† |
| 31 | †† |
| 32 | nt |
| 33 | †† |
| 34 | †† |
| 35 | †† |
| 36 | †† |
| 37 | nt |
| 38 | †† |
| 39 | ††† |
| 40 | ††† |
| 41 | †† |
| 42 | nt |
| 43 | †† |
| 44 | †† |
| 45 | †† |
| 46 | †† |
| 47 | †† |
| 48 | nt |
| 49 | nt |
| 50 | nt |
| 51 | nt |
| 52 | nt |
| 53 | nt |
| 54 | nt |
| 55 | nt |
| 56 | †† |
| 57 | †† |
| 58 | ††† |
| 59 | ††† |
| 60 | †† |
| 61 | †† |
| 62 | †† |
| 63 | †† |
| 64 | ††† |
| 65 | ††† |
| 66 | ††† |
| 67 | ††† |
| 68 | ††† |
| 69 | ††† |
| 70 | †† |
| 71 | ††† |
| 72 | †† |
| 73 | †† |
| 74 | †† |
| 75 | nt |
| 76 | †† |
| 77 | nt |
| 78 | ††† |
| 79 | ††† |
| 80 | ††† |
| 81 | ††† |
| 82 | †† |
| 83 | ††† |
| 84 | ††† |
| 85 | †† |
| 86 | ††† |
| 87 | ††† |
| 88 | ††† |
| 89 | ††† |
| 90 | ††† |
| 91 | †† |
| 92 | ††† |
| 93 | ††† |
| 94 | ††† |
| 95 | †† |
| 96 | † |
| 97 | †† |
| 98 | †† |
| 99 | ††† |
| 100 | ††† |
| 101 | †† |
| 102 | ††† |
| 103 | † |
| 104 | †† |
| 105 | ††† |
| 106 | ††† |
| 107 | ††† |
| 108 | ††† |
| 109 | †† |
| 110 | †† |
| 111 | † |
| 112 | †† |
| 113 | †† |
| 114 | ††† |
| 115 | †† |
| 116 | †† |
| 117 | ††† |
| 118 | †† |
| 119 | nt |
| 120 | †† |
| 121 | †† |
| 122 | ††† |
| 123 | † |
| 124 | † |
| 125 | nt |
| 126 | † |
| 127 | nt |
| 128 | †† |
| 129 | † |
| 130 | †† |
| 131 | † |
| 132 | †† |
| 133 | †† |
| 134 | † |
| 135 | † |
| 136 | ††† |
| 137 | †† |
| 138 | †† |
| 139 | †† |
| 140 | † |
| 141 | † |
| 142 | nt |
| 143 | †† |
| 144 | ††† |
| 145 | † |
| 146 | †† |
| 147 | nt |
| 148 | †† |
| 149 | †† |
| 150 | † |
| 151 | nt |
| 152 | †† |
| 153 | †† |
| 154 | †† |
| 155 | †† |
| 156 | ††† |
| 157 | ††† |
| 158 | ††† |
| 159 | † |
| 160 | †† |
| 161 | †† |
| 162 | † |
| 163 | nt |
| 164 | † |
| 165 | † |
| 166 | † |
| 167 | † |
| 168 | nt |
| 169 | † |
| 170 | nt |
| 171 | nt |
| 172 | †† |
| 173 | nt |

nt: not tested

What is claimed is:

1. A compound represented by formula (I): 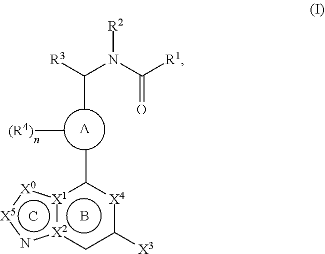
or a pharmaceutically acceptable salt thereof, wherein:
X⁰ is N, X¹ is C, X² is N, X⁴ is N and X⁵ is CH; X⁰ is CR⁰, X¹ is C, X² is N, X⁴ is N and X⁵ is CH; X⁰ is CR⁰, X¹ is N, X² is C, X⁴ is N and X⁵ is CH; X⁰ is CR⁰, X¹ is N, X² is C, X⁴ is CH and X⁵ is CH; X⁰ is CR⁰, X¹ is C, X² is N, X⁴ is CH and X⁵ is CH; or X⁰ is CH, X¹ is N, X² is C, X⁴ is CH and X⁵ is N;
X³ is H, —OR⁵, —N(R⁵)₂, 5- to 6-membered heteroaryl, or 4- to 7-membered monocyclic heterocyclyl, wherein the 5- to 6-membered heteroaryl and the 4- to 7-membered monocyclic heterocyclyl are optionally substituted with one or more R⁵⁰;
R⁰ is H, halo, methyl, halomethyl, cyclopropyl or CN;
Ring A is phenyl, 5 or 6-membered heteroaryl or 5 to 10-membered monocyclic or bicyclic heterocyclyl;
R¹ is selected from —N(R^{1a})₂, —OR^{1a}, phenyl, 3- to 7-membered monocyclic carbocyclyl, 3- to 7-membered monocyclic heterocyclyl, 5- to 6-membered heteroaryl, 7- to 10-membered bicyclic carbocyclyl, 7- to 10-membered bicyclic heterocyclyl, and 8- to 10-membered bicyclic heteroaryl, wherein the phenyl, 3- to 7-membered monocyclic carbocyclyl, 3- to 7-membered monocyclic heterocyclyl, 5- to 6-membered heteroaryl, 7- to 10-membered bicyclic carbocyclyl, and 7- to 10-membered bicyclic heterocyclyl represented by R¹ are each optionally substituted with one or more R¹⁰;
R^{1a}, for each occurrence, is independently selected from H, C₁₋₆ alkyl, C₂₋₆ alkenyl, C₂₋₆ alkynyl, phenyl, 3- to 7-membered carbocyclyl ring, 3- to 7-membered monocyclic heterocyclyl, and 5- to 6-membered heteroaryl, wherein the C₁₋₆ alkyl, C₂₋₆ alkenyl, C₂₋₆ alkynyl, phenyl, 3- to 7-membered carbocyclyl ring, 3- to 7-membered monocyclic heterocyclyl, and 5- to 6-membered heteroaryl represented by R^{1a} are each optionally substituted with one or more R¹⁰;
R¹⁰, for each occurrence, is independently selected from halogen, —OR^{10a}, —S(O)₂R^{10a}, —CN, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R¹⁰ are each optionally substituted with one or more R¹⁵;
R^{10a} is C₁₋₆ alkyl optionally substituted with one or more halogen;
R¹⁵, for each occurrence, is independently selected from halogen, C₁₋₆ alkyl, C₁₋₆ haloalkyl, —CN, and —OR^{5a};
R^{15a} is C₁₋₆ alkyl;
R² is H, C₁₋₆ alkyl, C₂₋₆ alkenyl, and C₂₋₆ alkynyl;
or R¹ and R², together with their intervening atoms, form a Ring D selected from 3- to 7-membered monocyclic heterocyclyl, 7- to 10-membered bicyclic heterocyclyl, and 8- to 10-membered bicyclic heteroaryl, wherein Ring D is optionally substituted with one or more R¹⁰⁰;
R¹⁰⁰, for each occurrence, is independently selected from C₁₋₆ alkyl, C₂₋₆ alkenyl, C₂₋₆ alkynyl, 3- to 7-membered monocyclic carbocyclyl, 4- to 6-membered monocyclic heterocyclyl and halogen; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R¹⁰⁰ are each optionally substituted with one or more R¹⁵⁰;
R¹⁵⁰, for each occurrence, is independently selected from halogen, —OR^{150a}, 3- to 7-membered carbocyclyl ring and 4- to 7-membered monocyclic heterocyclyl;
R^{150a} is H or C₁₋₆ alkyl;
R³ is selected from H, halogen, —C(O)N(R^{3a})₂, —C(O)OR^{3a}, —C(O)R^{3a}, C₁₋₆ alkyl, C₂₋₆ alkenyl, and C₂₋₆ alkynyl, wherein the C₁₋₆ alkyl, C₂₋₆ alkenyl, and C₂₋₆ alkynyl represented by R³ are each optionally substituted with one or more substituents selected from halogen and hydroxyl;
R^{3a}, for each occurrence, is independently selected from H, C₁₋₆ alkyl, C₂₋₆ alkenyl, C₂₋₆ alkynyl, phenyl, 3- to 7-membered carbocyclyl ring, 3- to 7-membered monocyclic heterocyclyl, or 5- to 6-membered heteroaryl, wherein C₁₋₆ alkyl, C₂₋₆ alkenyl, C₂₋₆ alkynyl, phenyl, 3- to 7-membered carbocyclyl ring, 3- to 7-membered monocyclic heterocyclyl, and 5- to 6-membered heteroaryl are optionally substituted with one or more R³⁰;
or two R^{3a} groups on the same nitrogen are taken together with their intervening atoms to form a ring selected from 3- to 7-membered monocyclic heterocyclyl and 5- to 6-membered heteroaryl, wherein said ring is optionally substituted with one or more R³⁰;
R³⁰, for each occurrence, is independently selected from halogen, —OR^{30a}, —N(R^{30a})₂, —C(O)N(R^{30a}), —C(O)₂R^{30a}, oxo, C₁₋₆ alkyl, C₂₋₆ alkenyl, C₂₋₆ alkynyl, 3- to 7-membered monocyclic carbocyclyl, and 4- to 6-membered monocyclic heterocyclyl;
R^{30a} is H or C₁₋₆ alkyl;
R⁴, for each occurrence, is independently selected from H, halogen, —NO₂, —CN, —OR^{4a}, —SR^{4a}, —N(R^{4a})₂, —C(O)R^{4a}, —C(O)OR^{4a}, —S(O)R^{4a}, —S(O)₂R^{4a}, —C(O)N(R^{4a})₂, —SO₂N(R^{4a})₂, —OC(O)R^{4a}, —N(R^{4a})C(O)R^{4a}, —N(R^{4a})C(O)OR^{4a}, —N(R^{4a})SO₂R^{4a}, —OC(O)N(R^{4a})₂, C₁₋₆ alkyl, C₂₋₆ alkenyl and C₂₋₆ alkynyl, wherein the C₁₋₆ alkyl, C₂₋₆ alkenyl and C₂₋₆ alkynyl are each optionally substituted with one or more R⁴⁰;
R^{4a} is H, C₁₋₆ alkyl, C₂₋₆ alkenyl, C₂₋₆alkynyl, phenyl, 3- to 8-membered carbocyclyl ring, 3- to 7-membered monocyclic heterocyclyl, and 5- to 6-membered heteroaryl, wherein the C₁₋₆ alkyl, C₂₋₆ alkenyl, C₂₋₆ alkynyl, phenyl, 3- to 8-membered carbocyclyl ring, 3- to 7-membered monocyclic heterocyclyl, and 5- to 6-membered heteroaryl represented by R^{4a} are each optionally substituted with one or more R⁴⁰;
or two R^{4a} groups on the same nitrogen are taken together with their intervening atoms to form a ring selected from 3- to 7-membered monocyclic heterocyclyl and 5- to 6-membered heteroaryl, wherein said ring is optionally substituted with one or more R⁴⁰;
R⁴⁰, for each occurrence, is independently selected from halogen, —OR^{40a}, —N(R^{40a})₂, —C(O)N(R^{40a})₂, —C(O)₂R^{40a}, oxo, C₁₋₆ alkyl, C₂₋₆ alkenyl, C₂₋₆ alkynyl, 3- to 7-membered monocyclic carbocyclyl, and 4- to 6-membered monocyclic heterocyclyl;
wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R⁴⁰ are each optionally substituted with one or more R⁴⁵;
R^{40a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl are each optionally substituted with one or more R⁴⁵;
R⁴⁵, for each occurrence, is independently selected from C₁₋₆ alkyl, halogen and —OR^{45a};
R^{45a} is H or C₁₋₆ alkyl;
or R³ and R⁴, together with their intervening atoms, form a Ring E, wherein Ring E is selected from 4- to 7-membered monocyclic carbocycle and 4- to 7-membered monocyclic heterocycle, wherein Ring E is optionally substituted with R³⁰⁰;
R³⁰⁰, for each occurrence, is independently selected from C₁₋₆ alkyl, C₂₋₆ alkenyl, C₂₋₆ alkynyl, 3- to 7-membered monocyclic carbocyclyl, 4- to 6-membered monocyclic heterocyclyl, halogen, —C(O)R^{300a}, —OR^{300a}, and —S(O)₂R^{300a}; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R³⁰⁰ are each optionally substituted with one or more R³⁵⁰;
R^{300a} is selected from H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R^{300a} are each optionally substituted with one or more R³⁵⁰;
R³⁵⁰, for each occurrence, is independently selected from C₁₋₆ alkyl, halogen, —CN, —C(O)R^{350a}, —C(O)N(R^{350a})₂, —N(R^{350a})₂, and —OR^{350a};
R^{350a}, for each occurrence, is independently H or C₁₋₆ alkyl optionally substituted with one to three halogen;
R⁵ is C₁₋₆ alkyl optionally substituted with one or more substituents independently selected from halo, C₁₋₆ alkoxy, and C₁₋₆ haloalkoxy;
R⁵⁰, for each occurrence, is independently selected from halogen, —OR^{50a}, —N(R^{50a})₂, —C(O)N(R^{50a}), —C(O)₂R^{50a}, oxo, C₁₋₆ alkyl, 3- to 7-membered monocyclic carbocyclyl, and 4- to 6-membered monocyclic heterocyclyl, wherein the C₁₋₆alkyl, the 3- to 7-membered monocyclic carbocyclyl, and the 4- to 6-membered monocyclic heterocyclyl represented by R⁵⁰ are each optionally substituted with one or more substituents independently selected from C₁₋₆alkyl, CN, halo and C₁₋₆ alkoxy;
R^{50a} is H or C₁₋₆ alkyl;
n is 0, 1, 2, 3 or 4.

2. The compound of claim 1, or a pharmaceutically acceptable salt thereof, wherein X³ is —OR⁵, —N(R⁵)₂, 5- to 6-membered heteroaryl, or 4- to 7-membered monocyclic heterocyclyl, wherein the 5- to 6-membered heteroaryl and the 4- to 7-membered monocyclic heterocyclyl are optionally substituted with one or more R⁵⁰.

3. The compound of claim 1 or 2, or a pharmaceutically acceptable salt thereof, wherein R⁰ is H.

4. The compound of claim 1 or 2, or a pharmaceutically acceptable salt thereof, wherein the compound is represented by the following formula: 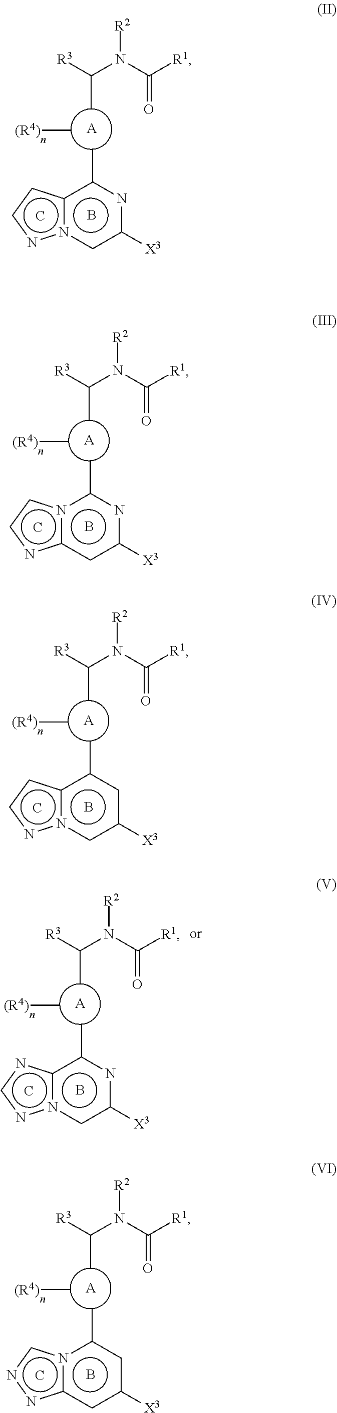
or a pharmaceutically acceptable salt thereof.

5. The compound of any one of claims 1-4, or a pharmaceutically acceptable salt thereof, wherein:
X³ is —OR⁵, —N(R⁵)₂, 5-membered heteroaryl, or 4- to 6-membered monocyclic heterocyclyl, wherein the 5-membered heteroaryl and the 4- to 6-membered monocyclic heterocyclyl are optionally substituted with one to three R⁵⁰; and
R⁵ is C₁₋₆ alkyl optionally substituted with C₁₋₆ alkoxy.

6. The compound of any one of claims 1-5, or a pharmaceutically acceptable salt thereof, wherein X³ is selected from phenyl, azetidine, morpholine, oxadiazole, piperazine, pyrazole, tetrazole, each optionally substituted with one or two R⁵⁰.

7. The compound of claim 6, or a pharmaceutically acceptable salt thereof, wherein X³ is selected from: 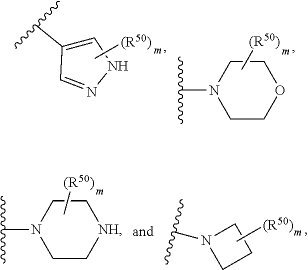
wherein m is 0, 1 or 2.

8. The compound of any one of claims 1-5, or a pharmaceutically acceptable salt thereof, wherein X³ is selected from —O—CH₂—CH₂—OCH₃, —N(CH₃)₂, 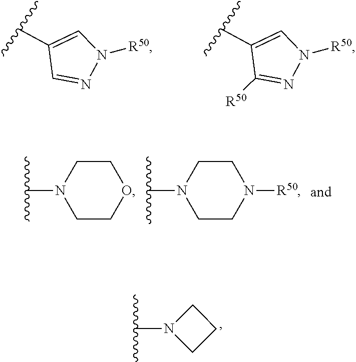
wherein [symbol] represents a bond to Ring B.

9. The compound of any one of claims 1-8, or a pharmaceutically acceptable salt thereof, wherein R⁵⁰ for each occurrence, is independently C₁₋₆ alkyl or a 4- to 6-membered monocyclic heterocyclyl, wherein the C₁₋₆alkyl represented by R⁵⁰ is optionally substituted with halo or CN.

10. The compound of claim 9, or a pharmaceutically acceptable salt thereof, wherein R⁵⁰, for each occurrence, is independently selected from —CH₃, —CH₂—CN and

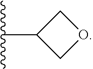 [SMILES: *C1COC1]

11. The compound of claim 9, or a pharmaceutically acceptable salt thereof, wherein R⁵⁰ is —CH₃.

12. The compound of any one of claims 1-11, or a pharmaceutically acceptable salt thereof, wherein Ring A is phenyl, 5 or 6-membered heteroaryl or 5 to 10-membered monocyclic or bicyclic heterocyclyl, each or which is optionally substituted with one to three R⁴.

13. The compound of claim 12, or a pharmaceutically acceptable salt thereof, wherein Ring A is selected from 3-azabicyclo[3.2.1]octane, azepane, phenyl, piperidine, pyridine and pyrrolidine, each of which is optionally substituted with one to three R⁴.

14. The compound of claim 12, or a pharmaceutically acceptable salt thereof, wherein Ring A is selected from: 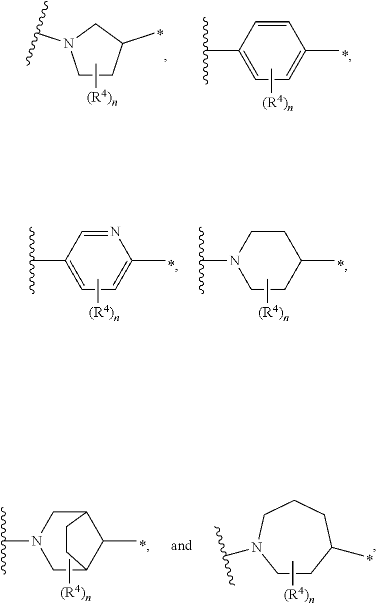
wherein [symbol] represents a bond to Ring B, and -* represents bond to

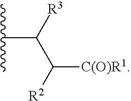

15. The compound of any one of claims 1-14, or a pharmaceutically acceptable salt thereof, wherein n is 0, 1, 2.

16. The compound of claim 12, or a pharmaceutically acceptable salt thereof, wherein Ring A is selected from: 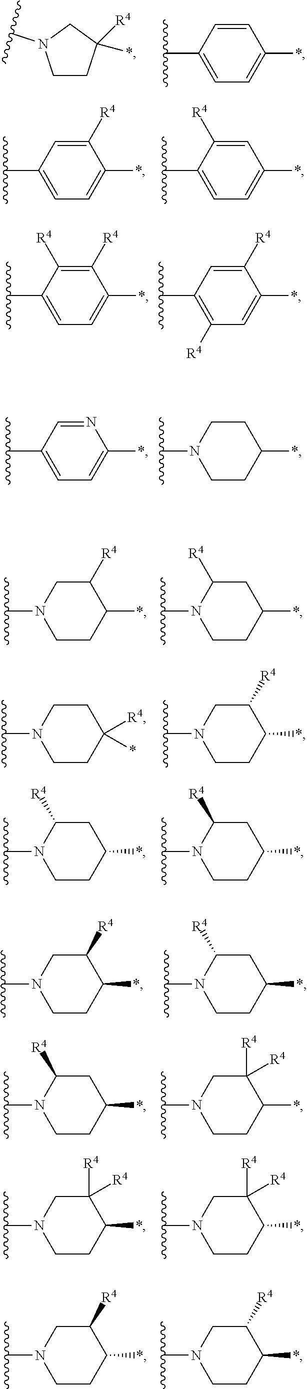 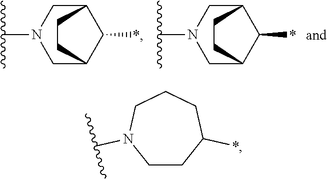
wherein [symbol] represents a bond to Ring B, and -* represents bond to

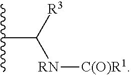

17. The compound of any one of claims 1-16, or a pharmaceutically acceptable salt thereof, wherein R⁴ is selected from halogen, C₁₋₆ alkyl and C₁₋₆ haloalkyl.

18. The compound of claim 17, or a pharmaceutically acceptable salt thereof, wherein R⁴ is selected from C₁, F, —CH₃ and —CHF₂.

19. The compound of any one of claims 1-18, or a pharmaceutically acceptable salt thereof, wherein R¹ is 5-membered heteroaryl optionally substituted with one or two R¹⁰.

20. The compound of claim 19, or a pharmaceutically acceptable salt thereof, wherein R¹ is selected from oxazole, oxadiazole, pyrazole, tetrazole and triazole, each of which is optionally substituted with one or two R¹⁰.

21. The compound of claim 19, or a pharmaceutically acceptable salt thereof, wherein R¹ is selected from:

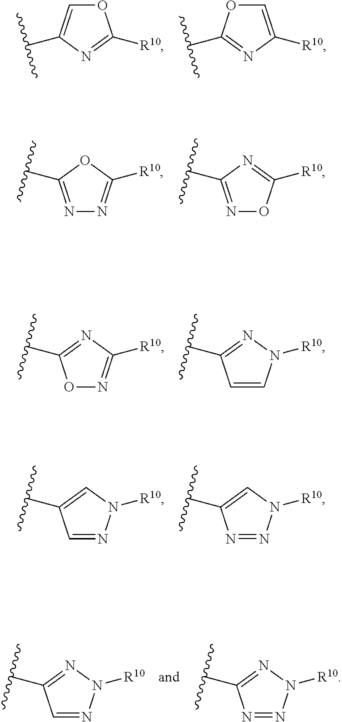

22. The compound of any one of claims 1-21, or a pharmaceutically acceptable salt thereof, wherein R¹⁰, for each occurrence, is independently selected from C₁₋₆ alkyl and C₃₋₆ cycloalkyl, each of which is optionally substituted with one to three R¹⁵.

23. The compound of claim 22, or a pharmaceutically acceptable salt thereof, wherein R¹⁰, for each occurrence, is independently selected from C₁₋₄ alkyl and cyclopropyl, each of which is optionally substituted with one or three R¹⁵.

24. The compound of any one of claims 1-23, or a pharmaceutically acceptable salt thereof, wherein R¹⁵, for each occurrence, is independently selected from halogen, C₁₋₄ alkyl and C₁₋₄ haloalkyl.

25. The compound of claim 24 or a pharmaceutically acceptable salt thereof, wherein R¹⁵, for each occurrence, is independently selected from F, —CH₃ and —CH₂F.

26. The compound of any one of claims 1-21, or a pharmaceutically acceptable salt thereof, wherein R¹⁰, for each occurrence, is independently selected from —C(CH₃)₃, —C(CH₃)₂—CH₂F,

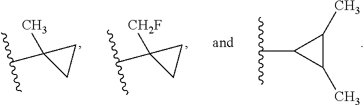

27. The compound of any one of claims 1-26, or a pharmaceutically acceptable salt thereof, wherein R² is H.

28. The compound of any one of claims 1-18, or a pharmaceutically acceptable salt thereof, wherein R¹ and R², together with their intervening atoms, form a Ring D selected from 5- to 7-membered monocyclic heterocyclyl and 7- to 10-membered bicyclic heterocyclyl, wherein Ring D is optionally substituted with one or more R¹⁰⁰.

29. The compound of claim 28, or a pharmaceutically acceptable salt thereof, wherein Ring D is selected from piperazinone and dihydropyrrolo[3,4-d]thiazolone, wherein Ring D is optionally substituted with one or two R¹⁰⁰.

30. The compound of claim 28, or a pharmaceutically acceptable salt thereof, wherein Ring D is selected from: 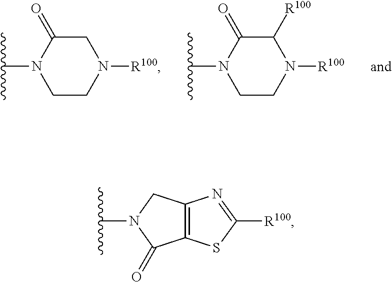
wherein [symbol] represents a bond to —C(R³)—Ring A.

31. The compound of any one of claims 1-30, or a pharmaceutically acceptable salt thereof, wherein:
R¹⁰⁰, for each occurrence, is independently selected from C₁₋₆ alkyl and 4- to 6-membered monocyclic heterocyclyl, each of which is optionally substituted with one or two R¹⁵⁰; and
R¹⁵⁰, for each occurrence, is independently selected from C₃₋₆ cycloalkyl and 4- to 6-membered monocyclic heterocyclyl.

32. The compound of claim 31, or a pharmaceutically acceptable salt thereof, wherein:
R¹⁰⁰, for each occurrence, is independently selected from C₁₋₆ alkyl and oxetanyl, wherein the C₁₋₆ alkyl represented by R¹⁰⁰ is optionally substituted with R¹⁵⁰; and
R¹⁵⁰, for each occurrence, is independently selected from cyclobutyl and oxetanyl.

33. The compound of claim 31, or a pharmaceutically acceptable salt thereof, wherein R¹⁰⁰, for each occurrence, is independently selected from —CH₃, —CH₂—CH₂—CH₃, —CH(CH₃)₂, —C(CH₃)₃, —CH₂—CH(CH₃)₂, —CH₂—C(CH₃)₃,

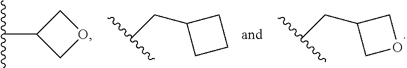 [SMILES: *C1COC1 ; *CC1CCC1 ; "and" ; *CC1COC1 ; "."]

34. The compound of any one of claims 1-33, or a pharmaceutically acceptable salt thereof, wherein R³ is selected from H and C₁₋₆ alkyl.

35. The compound of claim 34, or a pharmaceutically acceptable salt thereof, wherein R³ is H or —CH₃.

36. The compound of any one of claims 1-16 and 18-33, or a pharmaceutically acceptable salt thereof, wherein R³ and R⁴, together with their intervening atoms, form a Ring E, wherein Ring E is selected from 4- to 7-membered monocyclic carbocycle and 4- to 7-membered monocyclic heterocycle, wherein Ring E is optionally substituted with R³⁰⁰.

37. The compound of claim 36, or a pharmaceutically acceptable salt thereof, wherein Ring E is 5- to 7-membered monocyclic heterocycle optionally substituted with R³⁰⁰.

38. The compound of claim 36, or a pharmaceutically acceptable salt thereof, wherein Ring E is represented by the following structural formula: 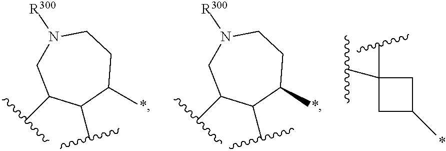
wherein [symbol] represents a point of fusion to Ring A and -* represents a bond to —N(R²)—C(O)—R¹.

39. The compound of any one of claims 1-38, or a pharmaceutically acceptable salt thereof, wherein:
R³⁰⁰, for each occurrence, is independently selected from C₁₋₆ alkyl and 4- to 6-membered monocyclic heterocyclyl, wherein the C₁₋₆ alkyl and 4- to 6-membered monocyclic heterocyclyl represented by R³⁰⁰ are each optionally substituted with one to three R³⁵⁰; and
R³⁵⁰, for each occurrence, is independently halogen.

40. The compound of claim 39, or a pharmaceutically acceptable salt thereof, wherein R³⁰⁰, for each occurrence, is independently selected from —CH₂—CF₃ and

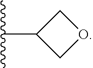 [SMILES: *C1COC1]

41. The compound of claim 1 or 2, or a pharmaceutically acceptable salt thereof, wherein the compound is represented by the following formula: 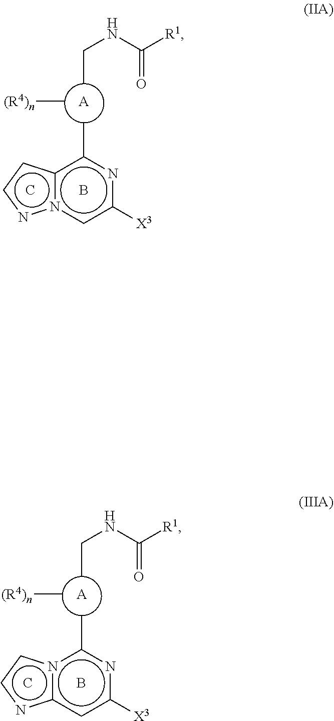
or a pharmaceutically acceptable salt thereof, wherein:
R¹ is a 5-membered heteroaryl optionally substituted with R¹⁰;
R¹⁰ is C₁₋₄ alkyl, C₁₋₄ haloalkyl or C₃₋₆ cycloalkyl optionally substituted with C₁₋₃ alkyl;
X³ is a 5-membered heteroaryl or a 6-membered monocyclic saturated heterocyclyl, each of which is optionally substituted with R⁵⁰;
R⁵⁰ is C₁₋₃ alkyl or C₁₋₃ haloalkyl;
Ring A is phenyl, 6-membered heteroaryl or 6-membered monocyclic saturated heterocyclyl;
R⁴, for each occurrence, is independently selected from halogen, C₁₋₃ alkyl and C₁₋₃ haloalkyl; and
n is 0, 1 or 2.

42. The compound of claim 41, or a pharmaceutically acceptable salt thereof, wherein:
R¹ is selected from oxadiazole, triazole and tetrazole, each of which is optionally substituted with R¹⁰;
X³ is pyrazole or piperazine, each of which is optionally substituted with R⁵⁰; and
Ring A is phenyl, pyridine or piperidine.

43. The compound of claim 41, or a pharmaceutically acceptable salt thereof, wherein:
R¹ is selected from: 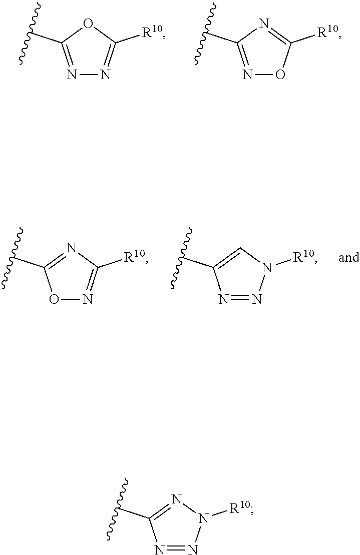
X³ is selected from: 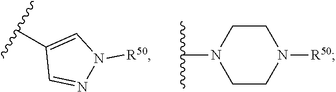
and
Ring A is selected from: 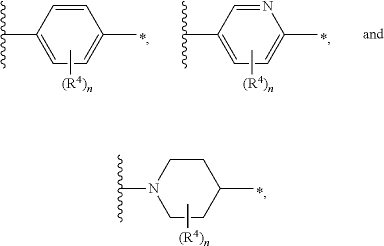
wherein [symbol] represents a bond to Ring B, and -* represents bond to

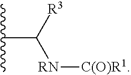

44. The compound of claim 43, or a pharmaceutically acceptable salt thereof, wherein ring A is selected from: 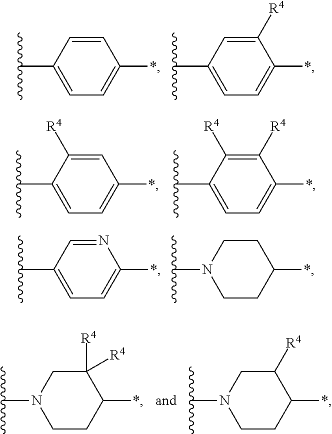
wherein [symbol] represents a bond to Ring B, and -* represents bond to

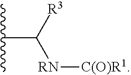

45. The compound of any one of claims 41-44, or a pharmaceutically acceptable salt thereof, wherein:
R¹⁰ is —C(CH₃)₃ or 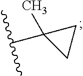 [SMILES: *C1(C)CC1]
R⁴, for each occurrence, is independently selected from F, —CH₃ and —CHF₂; and
R⁵⁰ is —CH₃.

46. A pharmaceutical composition comprising a compound of any one of claims 1-45 or a pharmaceutically acceptable salt thereof, and a pharmaceutically acceptable excipient.

47. A method of treating a disorder responsive to inhibition of Bruton's tyrosine kinase in a subject comprising administering to the subject an effective amount of the compound according to any of claims 1-45, or a pharmaceutically acceptable salt thereof, or a pharmaceutical composition according to claim 46.

48. The method of claim 47, wherein the disorder is an autoimmune disorder.

49. The method of claim 48, wherein the autoimmune disorder is rheumatoid arthritis.

50. The method of claim 48, wherein the autoimmune disorder is systemic lupus erythematosus.

51. The method of claim 47, wherein the disorder is atopic dermatitis.

52. The method of claim 51, wherein the disorder is leukemia or lymphoma.
